SEQUENCE LISTING (169 sequences)

SEQ ID NO: 1 - protein, 234 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSSDKTHTC

SEQ ID NO: 2 - protein, 215 aa, synthetic construct
QTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGQPKSSPSVTLFPPSSEELETNKATLVCTITDFYPGVVTVDWKVDGTPVTQGMETTQPSKQSNNKYMASSYLTLTARAWERHSSYSCQVTHEGHTVEKSLSRADSS

SEQ ID NO: 3 - protein, 215 aa, synthetic construct
QTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLSQPKAAPSVTLFPPSSEELQANKATLVCLVSDFYPGAVTVAWKADGSPVKVGVETTKPSKQSNNKYAASSYLSLTPEQWKSHRSYSCRVTHEGSTVEKTVAPAESS

SEQ ID NO: 4 - protein, 229 aa, synthetic construct
QVQLVQSGAEVKKPGASVKVSCKASGYTFISYTMHWVRQAPGQGLEWMGYINPRSGYTHYNQKLKDKATLTADKSASTAYMELSSLRSEDTAVYYCARSAYYDYDGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSSDKTHTC

SEQ ID NO: 5 - protein, 235 aa, synthetic construct
MDMRVPAQLLGLLLLWLPGAKCDIQMTQSPSSLSASVGDRVTITCSASSSVSYMNWYQQKPGKAPKRLIYDTSKLASGVPSRFSGSGSGTDFTLTISSLQPEDFATYYCQQWSSNPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGES

SEQ ID NO: 6 - protein, 230 aa, synthetic construct
QVQLVESGGGVVQPGRSLRLSCAASGFTFSDFGMNWVRQAPGKGLEWVALIYYDGSNKFYADSVKGRFTISRDNSKNTLYLQMNSLRAEDTAVYYCAKPHYDGYYHFFDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSSDKTHTC

SEQ ID NO: 7 - protein, 213 aa, synthetic construct
DIQMTQSPSSLSASVGDRVTITCKGSQDINNYLAWYQQKPGKAPKLLIYNTDILHTGVPSRFSGSGSGTDFTFTISSLQPEDIATYYCYQYNNGYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGES

SEQ ID NO: 8 - protein, 227 aa, synthetic construct
QVQLVQSGAEDKKPGASVKVSCKASGFNIKDTYIHWVRQAPGQGLEWMGRIDPANDNTLYASKFQGRVTITADTSSNTAYMELSSLRSEDTAVYYCGRGYGYYVFDHWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSSDKTHTC

SEQ ID NO: 9 - protein, 214 aa, synthetic construct
DIVMTQSPSSLSASVGDRVTITCRTSRSISQYLAWYQEKPGKAPKLLIYSGSTLQSGVPSRFSGSGSGTDFTLTISSLQPEDFATYYCQQHNENPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGES

SEQ ID NO: 10 - protein, 231 aa, synthetic construct
QVQLQQSGPEVVKPGASVKMSCKASGYTFTSYVIHWVRQKPGQGLDWIGYINPYNDGTDYDEKFKGKATLTSDTSTSTAYMELSSLRSEDTAVYYCAREKDNYATGAWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSSDKTHTC

SEQ ID NO: 11 - protein, 219 aa, synthetic construct
DIVMTQSPDSLAVSLGERVTMNCKSSQSLLYSTNQKNYLAWYQQKPGQSPKLLIYWASTRESGVPDRFSGSGSGTDFTLTISSVQAEDVAVYYCQQYYSYRTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGES

SEQ ID NO: 12 - protein, 227 aa, synthetic construct
EIQLVQSGGGLVKPGGSVRISCAASGYTFTNYGMNWVRQAPGKGLEWMGWINTHTGEPTYADSFKGRFTFSLDDSKNTAYLQINSLRAEDTAVYFCTRRGYDWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSSDKTHTC

SEQ ID NO: 13 - protein, 214 aa, synthetic construct
DIQMTQSPSSLSASVGDRVTITCRASQDINSYLSWFQQKPGKAPKTLIYRANRLESGVPSRFSGSGSGTDYTLTISSLQYEDFGIYYCQQYDESPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGES

SEQ ID NO: 14 - protein, 226 aa, synthetic construct
EVQLVESGGGLVKPGGSLKLSCAASGLTFSSYAMSWVRQTPEKRLEWVASISSGGFTYYPDSVKGRFTISRDNARNILYLQMSSLRSEDTAMYYCARDEVRGYLDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTC

SEQ ID NO: 15 - protein, 214 aa, synthetic construct
DIQMTQTTSSLSASLGDRVTISCSASQGISNYLNWYQQKPDGTVKLLIYYTSSLHSGVPSRFSGSGSGTDYSLTISNLEPEDIATYYCQQYSKLPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - protein, 226 aa, synthetic construct
EVQLVESGGGLVMPGGSLKLSCAASGFAFSSYDMSWVRQTPEKRLEWVAYISGGGFTYYPDTVKGRFTLSRDNAKNTLYLQMSSLKSEDTAMYYCARQGANWELVYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSSDKTHTC

SEQ ID NO: 17 - protein, 214 aa, synthetic construct
DIVMTQSPATLSVTPGDRVFLSCRASQSISDFLHWYQQKSHESPRLLIKYASQSISGIPSRFSGSGSGSDFTLSINSVEPEDVGVYFCQNGHNFPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGES

SEQ ID NO: 18 - protein, 227 aa, synthetic construct
QVQLQQPGAELVRPGSSVKLSCKASGYTFTRYWIHWVKQRPIQGLEWIGNIDPSDSETHYNQKFKDKATLTVDKSSGTAYMQLSSLTSEDSAVYYCATEDLYYAMEYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSSDKTHTC

SEQ ID NO: 19 - protein, 219 aa, synthetic construct
NIMMTQSPSSLAVSAGEKVTMTCKSSQSVLYSSNQKNYLAWYQQKPGQSPKLLIYWASTRESGVPDRFTGSGSGTDFTLTISSVQPEDLAVYYCHQYLSSHTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGES

SEQ ID NO: 20 - protein, 229 aa, synthetic construct
QVQLQQPGTELVRPGSSVKLSCKASGYTFTSYWVNWVKQRPDQGLEWIGRIDPYDSETHYNQKFTDKAISTIDTSSNTAYMQLSTLTSDASAVYYCSRSPRDSSTNLADWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSSDKTHTC

SEQ ID NO: 21 - protein, 214 aa, synthetic construct
DIVMTQSPATLSVTPGDRVSLSCRASQSISDYLHWYQQKSHESPRLLIKYASQSISGIPSRFSGSGSGSDFTLSINSVEPEDVGVYYCQNGHSFPLTFGAGTKLELRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGES

SEQ ID NO: 22 - protein, 227 aa, synthetic construct
QVQLVQSGAEDKKPGASVKVSCKASGFNIKDTYIHWVRQAPGQGLEWMGRIDPANDNTLYASKFQGRVTITADTSSNTAYMELSSLRSEDTAVYYCGRGAGAYVFDHWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSSDKTHTC

SEQ ID NO: 23 - protein, 214 aa, synthetic construct
DIVMTQSPSSLSASVGDRVTITCRTSRSISAALAWYQEKPGKAPKLLIYSGSTLQSGVPSRFSGSGSGTDFTLTISSLQPEDFATYYCQQHNENPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGES

SEQ ID NO: 24 - protein, 494 aa, synthetic construct
METDTLLLWVLLLWVPGSTGDYKAKEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSGGGSGGGSGGGGSSDIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGGGGSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSAEPPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 25 - DNA, 1491 bp, synthetic construct
  source 1..1491: mol_type = other DNA
atggagaccgacaccctgttgctttgggtactgttactttgggtgcccggatctaccggtgattacaaggccaaggaggtgcagctgcagcagagcggagccgagctggtgaagccaggcgcttccgtgaagatgtcttgtaaggcctccggctacacattcaccagctacaatatgcactgggtaaagcagactccggggcagggcctggagtggataggtgccatctaccctggcaacggcgacaccagctacaaccagaagtttaaggggaaggctactctaacagcggacaagtcgtcctctaccgcctacatgcaactcagctccctgacgagcgaggactccgcggactactactgtgcccgctccaactactacggctctagctattggttcttcgacgtgtggggcgctggaacgaccgtgaccgtgtcttccggtggaggttccgggggcggaagcggcggtggcggcagttcggacatcgtgctgacccagagccctgccatcctgtccgcttccccgggggagaaagttacgatgacctgccgagcgagctccagtgtcaactacatggattggtaccagaagaagcccggcagcagtcccaagccgtggatttacgctactagcaacctggcgtccggtgtcccggctcgcttctcaggttctggctcgggtactagttattcattaaccatttctcgcgtggaggctgaggacgctgccacctactactgccaacagtggtctttcaaccctcccactttcggaggcggcaccaagctcgagatcaagggcgggggtggctccgcagcagccattgaggtgatgtatcctcctccctatttggacaacgagaagtcaaatggcaccatcatccacgttaagggcaagcacctgtgcccatctcccctgttcccaggcccctctaagcccttctgggtcctggtggtggtcggcggcgtcctggcatgttactctctgctggtgaccgtcgcgttcatcatcttttgggtccggtccaagcgcagccgcctgctccactccgactacatgaatatgactcctcgtaggcccggtccaacccgcaagcactaccagccgtacgcgccgcccagagactttgctgcttaccgatccagagtgaaattttctaggtcggccgaacctcccgcatatcagcagggccagaaccagctgtacaacgaactcaacttgggacggcgcgaggaatacgatgtgctggataaacgccgtggccgcgatcccgagatgggcgggaagccacgtcgcaaaaaccctcaggagggcctttacaacgagttgcagaaggacaaaatggcggaggcctactccgagatcggaatgaagggggagcgccggcgcggcaaagggcatgacggcctctaccagggcctgtccacagccacgaaagacacctatgacgccctgcatatgcaggccctgcccccgcgctgataatga

SEQ ID NO: 26 - protein, 248 aa, synthetic construct
QVQLQQSGPGLVKPSQTLSLTCAISGDSVSSNSAAWNWIRQSPSNWIRQSPSGLEWLGRTYYRSKWYNDYAVSVKSRITINPDTSKNQFSLQLNSVTPEDTAVYYCARENIAAWTWAFDIWGQGTMVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTCGGHHHHHH

SEQ ID NO: 27 - protein, 214 aa, synthetic construct
EIVMTQSPGTLSLSPGERATLSCRASQSVSSSYLAWYQQKPGLAPRLLIYDTSLRATDIPDRFSGSGSGTAFTLTISRLEPEDFAVYYCQQYGSSPTFGQGTKVEIKRTVAAPSVFIFPPSDEQLKSGTASVVCLLNNFYPREAKVQWKVDNALQSGNSQESVTEQDSKDSTYSLCSTLTLSKADYEKHKVYACEVTHQGLSSPVTKSFNRGES

SEQ ID NO: 28 - protein, 238 aa, synthetic construct
EVQLVQSGAEVKKPGSSVKVSCKASGGTFTGYYMHWVRQAPGQGLEWMGWINPNSGGTNYAQKFQGRVTMTRDTSISTAYMELSRLRSDDTAVYYCARDLSSGYSGYFDYWGQGTLVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTCGGHHHHHH

SEQ ID NO: 29 - protein, 219 aa, synthetic construct
DVVMTQSPLSLPVTPGEPASISCRSSQSLLHSNGYNYLNWYLQKPGQSPQLLIYLGSNRASGVPDRFSGSGSGTDFTLKISRVEGEDVGDYYCMQALQSPFTFGQGTKLEIKRTVAAPSVFIFPPSDEQLKSGTASVVCLLNNFYPREAKVQWKVDNALQSGNSQESVTEQDSKDSTYSLCSTLTLSKADYEKHKVYACEVTHQGLSSPVTKSFNRGES

SEQ ID NO: 30 - protein, 238 aa, synthetic construct; identical to SEQ ID NO: 28

SEQ ID NO: 31 - protein, 219 aa, synthetic construct
DVVMTQSPLSLPVTPGEPASISCRSSQSLLHSNGYNFLDWYLQKPGQSPQLLIYLGSNRASGVPDRFSGSGSGTDFTLKISRVEADDVGVYYCMQSLQTPWTFGHGTKVEIKRTVAAPSVFIFPPSDEQLKSGTASVVCLLNNFYPREAKVQWKVDNALQSGNSQESVTEQDSKDSTYSLCSTLTLSKADYEKHKVYACEVTHQGLSSPVTKSFNRGES

SEQ ID NO: 32 - protein, 233 aa, synthetic construct
QVQLVESGGGVVQPGRSLRLSCAASGFTFSSFGMHWVRQAPGKGLEWVAYISSGSFTIYYADSVKGRFTISRDNSKNTLYLQMNSLRAEDTAVYYCARMRKGYAMDYWGQGTLVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTCHHHHHH

SEQ ID NO: 33 - protein, 219 aa, synthetic construct
DVVMTQSPLSLPVTLGQPASISCRSSQIIIHSDGNTYLEWFQQRPGQSPRRLIYKVSNRFSGVPDRFSGSGSGTDFTLKISRVEAEDVGVYYCFQGSHVPHTFGQGTKVEIKRTVAAPSVFIFPPSDEQLKSGTASVVCLLNNFYPREAKVQWKVDNALQSGNSQESVTEQDSKDSTYSLCSTLTLSKADYEKHKVYACEVTHQGLSSPVTKSFNRGES

SEQ ID NO: 34 - protein, 232 aa, synthetic construct
EVKLVESGGGLVKPGGSLKLSCAASGFTFSSYDMSWVRQTPEKRLEWVASISGGGFLYYLDSVKGRFTISRDNARNILYLHMTSLRSEDTAMYYCARSSYGEIMDYWGQGTSVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTCHHHHHH

SEQ ID NO: 35 - protein, 214 aa, synthetic construct
DILLTQSPAILSVSPGERVSFSCRASQRIGTSIHWYQQRTTGSPRLLIKYASESISGIPSRFSGSGSGTDFTLSINSVESEDVADYYCQQSHGWPFTFGGGTKLEIERTVAAPSVFIFPPSDEQLKSGTASVVCLLNNFYPREAKVQWKVDNALQSGNSQESVTEQDSKDSTYSLCSTLTLSKADYEKHKVYACEVTHQGLSSPVTKSFNRGES

SEQ ID NO: 36 - protein, 233 aa, synthetic construct
QVQLQQPGAELVKPGASVKLSCKASGYTFSSYWMHWVKQRPGQVLEWIGEINPGNGHTNYNEKFKSKATLTVDKSSSTAYMQLSSLTSEDSAVYYCARSFTTARGFAYWGQGTLVTVSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTCHHHHHH

SEQ ID NO: 37 - protein, 214 aa, synthetic construct
DIVMTQSPATQSVTPGDRVSLSCRASQTISDYLHWYQQKSHESPRLLIKYASQSISGIPSRFSGSGSGSDFTLSINSVEPEDVGVYYCQDGHSFPPTFGGGTKLEIKRTVAAPSVFIFPPSDEQLKSGTASVVCLLNNFYPREAKVQWKVDNALQSGNSQESVTEQDSKDSTYSLCSTLTLSKADYEKHKVYACEVTHQGLSSPVTKSFNRGES

SEQ ID NO: 38 - protein, 234 aa, synthetic construct; identical to SEQ ID NO: 1

SEQ ID NO: 39 - protein, 215 aa, synthetic construct; identical to SEQ ID NO: 3

SEQ ID NO: 40 - protein, 234 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTC

SEQ ID NO: 41 - protein, 215 aa, synthetic construct
QTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLSQPKAAPSVTLFPPSSEELQANKATLVCLVSDFYPGAVTVAWKADGSPVKVGVETTKPSKQSNNKYAASSYLSLTPEQWKSHRSYSCRVTHEGSTVEKTVAPAECS

SEQ ID NO: 42 - protein, 226 aa, synthetic construct
EVQLVESGGGLVMPGGSLKLSCAASGFAFSSYDMSWVRQTPEKRLEWVAYISGGGFTYYPDTVKGRFTLSRDNAKNTLYLQMSSLKSEDTAMYYCARQGANWELVYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTC

SEQ ID NO: 43 - protein, 214 aa, synthetic construct
DIVMTQSPATLSVTPGDRVFLSCRASQSISDFLHWYQQKSHESPRLLIKYASQSISGIPSRFSGSGSGSDFTLSINSVEPEDVGVYFCQNGHNFPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 44 - protein, 227 aa, synthetic construct
QVQLQQPGAELVRPGSSVKLSCKASGYTFTRYWIHWVKQRPIQGLEWIGNIDPSDSETHYNQKFKDKATLTVDKSSGTAYMQLSSLTSEDSAVYYCATEDLYYAMEYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTC

SEQ ID NO: 45 - protein, 219 aa, synthetic construct
NIMMTQSPSSLAVSAGEKVTMTCKSSQSVLYSSNQKNYLAWYQQKPGQSPKLLIYWASTRESGVPDRFTGSGSGTDFTLTISSVQPEDLAVYYCHQYLSSHTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 46 - protein, 240 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTCHHHHHH

SEQ ID NO: 47 - protein, 215 aa, synthetic construct
QTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLSQPKAAPSVTLFPPSSEELQANKATLVCLVSDFYPGAVTVAWKADGSPVKVGVETTKPSKQSNNKYAACSYLSLTPEQWKSHRSYSCRVTHEGSTVEKTVAPAESS

SEQ ID NO: 48 - protein, 234 aa, synthetic construct
QVQLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDNYSLDYWGQGTTLTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTCHHHHHH

SEQ ID NO: 49 - protein, 213 aa, synthetic construct
QIVLTQSPSSLSASLGEKVTMTCRASSSVSYMNWYQQKPGTSPKRWIYDTSKVASGVPDRFSGSGSGTSYSLTISSMEAEDAATYYCQQWSSNPLTFGAGTKLELKRTVAAPSVFIFPPSDEQLKSGTASVVCLLNNFYPREAKVQWKVDNALQSGNSQESVTEQDSKDSTYSLCSTLTLSKADYEKHKVYACEVTHQGLSSPVTKSFNRGES

SEQ ID NO: 50 - protein, 234 aa, synthetic construct
EVQLLESGGGLVQPGGSLRLSCAASGFTFSSFPMAWVRQAPGKGLEWVSTISTSGGRTYYRDSVKGRFTISRDNSKNTLYLQMNSLRAEDTAVYYCAKFRQYSGGFDYWGQGTLVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTCHHHHHH

SEQ ID NO: 51 - protein, 216 aa, synthetic construct
DIQLTQPNSVSTSLGSTVKLSCTLSSGNIENNYVHWYQLYEGRSPTTMIYDDDKRPDGVPDRFSGSIDRSSNSAFLTIHNVAIEDEAIYFCHSYVSSFNVFGGGTKLTVLGQPKANPTVTLFPPSSEELQANKATLVCLISDFYPGAVTVAWKADGSPVKAGVETTKPSKQSNNKYAACSYLSLTPEQWKSHRSYSCQVTHEGSTVEKTVAPTESS

SEQ ID NO: 52 - protein, 237 aa, synthetic construct
EVQLVESGGGLVQPGGSLRLSCAASGYSFTGYTMNWVRQAPGKGLEWVALINPTKGVSTYNQKFKDRFTISVDKSKNTAYLQMNSLRAEDTAVYYCARSGYYGDSDWYFDVWGQGTLVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTCHHHHHH

SEQ ID NO: 53 - protein, 214 aa, synthetic construct
DIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKRTVAAPSVFIFPPSDEQLKSGTASVVCLLNNFYPREAKVQWKVDNALQSGNSQESVTEQDSKDSTYSLCSTLTLSKADYEKHKVYACEVTHQGLSSPVTKSFNRGES

SEQ ID NO: 54 - protein, 234 aa, synthetic construct
QVQLVQSGGGVVQPGRSLRLSCKASGYTFTRYTMHWVRQAPGKGLEWIGYINPSRGYTNYNQKVKDRFTISRDNSKNTAFLQMDSLRPEDTGVYFCARYYDDHYCLDYWGQGTPVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTCHHHHHH

SEQ ID NO: 55 - protein, 213 aa, synthetic construct
DIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITRTVAAPSVFIFPPSDEQLKSGTASVVCLLNNFYPREAKVQWKVDNALQSGNSQESVTEQDSKDSTYSLCSTLTLSKADYEKHKVYACEVTHQGLSSPVTKSFNRGES

SEQ ID NO: 56 - protein, 230 aa, synthetic construct
QVQLVESGGGVVQPGRSLRLSCAASGFTFSDFGMNWVRQAPGKGLEWVALIYYDGSNKFYADSVKGRFTISRDNSKNTLYLQMNSLRAEDTAVYYCAKPHYDGYYHFFDSWGQGTLVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTC

SEQ ID NO: 57 - protein, 213 aa, synthetic construct
DIQMTQSPSSLSASVGDRVTITCKGSQDINNYLAWYQQKPGKAPKLLIYNTDILHTGVPSRFSGSGSGTDFTFTISSLQPEDIATYYCYQYNNGYTFGQGTKVEIKRTVAAPSVFIFPPSDEQLKSGTASVVCLLNNFYPREAKVQWKVDNALQSGNSQESVTEQDSKDSTYSLCSTLTLSKADYEKHKVYACEVTHQGLSSPVTKSFNRGES

SEQ ID NO: 58 - protein, 226 aa, synthetic construct
EVQLVESGGGLVQPGASLKLSCVASGFTFSDYWMSWVRQTPGKPMEWIGHIKYDGSYTNYAPSLKNRFTISRDNAKTTLYLQMSNVRSEDSATYYCAREAPGAASYWGQGTLVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTC

SEQ ID NO: 59 - protein, 219 aa, synthetic construct
DVVLTQTPVAQPVTLGDQASISCRSSQSLVHSNGNTYLEWFLQKPGQSPQLLIYKVSNRFSGVPDRFIGSGSGSDFTLKISRVEPEDWGVYYCFQGTHDPYTFGAGTKLELKRTVAAPSVFIFPPSDEQLKSGTASVVCLLNNFYPREAKVQWKVDNALQSGNSQESVTEQDSKDSTYSLCSTLTLSKADYEKHKVYACEVTHQGLSSPVTKSFNRGES

SEQ ID NO: 60 - protein, 229 aa, synthetic construct
EVQLQQSGAELVKPGASVKLSCRTSGFNIKDTYIHWVKQRPEQGLKWIGRIDPANGNTKYDPKFQDKATVTADTSSNTAYLQLSSLTSEDTAVYYCVTYAYDGNWYFDVWGAGTAVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTC

SEQ ID NO: 61 - protein, 214 aa, synthetic construct
DIKMTQSPSSMYVSLGERVTITCKASQDINSFLSWFQQKPGKSPKTLIYRANRLVDGVPSRFSGSGSGQDYSLTISSLEYEDMEIYYCLQYDEFPYTFGGGTKLEMKRTVAAPSVFIFPPSDEQLKSGTASVVCLLNNFYPREAKVQWKVDNALQSGNSQESVTEQDSKDSTYSLCSTLTLSKADYEKHKVYACEVTHQGLSSPVTKSFNRGES

SEQ ID NO: 62 - protein, 230 aa, synthetic construct
QVQLQQPGAELVRPGTSVKLSCKASGYTFTSYWMHWIKQRPEQGLEWIGRIDPYDSETHYNEKFKDKAILSVDKSSSTAYIQLSSLTSDDSAVYYCSRRDAKYDGYALDYWGQGTSVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTC

SEQ ID NO: 63 - protein, 219 aa, synthetic construct
DIVMTQAAPSVPVTPGESVSISCRSSKTLLHSNGNTYLYWFLQRPGQSPQVLIYRMSNLASGVPNRFSGSGSETTFTLRISRVEAEDVGIYYCMQHLEYPYTFGGGTKLEIERTVAAPSVFIFPPSDEQLKSGTASVVCLLNNFYPREAKVQWKVDNALQSGNSQESVTEQDSKDSTYSLCSTLTLSKADYEKHKVYACEVTHQGLSSPVTKSFNRGES

SEQ ID NO: 64 - protein, 236 aa, synthetic construct
EVQLVESGGGLVQPGGSLRLSCAASGYSFTGHWMNWVRQAPGKGLEWVGMIHPSDSETRYNQKFKDRFTISVDKSKNTLYLQMNSLRAEDTAVYYCARGIYFYGTTYFDYWGQGTLVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTCHHHHHH

SEQ ID NO: 65 - protein, 214 aa, synthetic construct
DIQMTQSPSSLSASVGDRVTITCRASKTISKYLAWYQQKPGKAPKLLIYSGSTLQSGVPSRFSGSGSGTDFTLTISSLQPEDFATYYCQQHNEYPLTFGQGTKVEIKRTVAAPSVFIFPPSDEQLKSGTASVVCLLNNFYPREAKVQWKVDNALQSGNSQESVTEQDSKDSTYSLCSTLTLSKADYEKHKVYACEVTHQGLSSPVTKSFNRGES

SEQ ID NO: 66 - protein, 235 aa, synthetic construct
EVQLVESGGDLVQPGRSLRLSCAASGFTFSDYYMSWVRQAPGKGLEWVAAIDNDGGSISYPDTVKGRFTISRDNAKNSLYLQMNSLRVEDTALYYCARQGRLRRDYFDYWGQGTLVTVSTASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTCHHHHHH

SEQ ID NO: 67 - protein, 218 aa, synthetic construct
DIQMTQSPSSLSASVGDRVTITCRASESVDSYGNSFMHWYQQKPGKAPKLLIYRASNLESGVPSRFSGSGSGTDFTFTISSLQPEDIATYYCQQSNEDPLTFGQGTKLEIKRTVAAPSVFIFPPSDEQLKSGTASVVCLLNNFYPREAKVQWKVDNALQSGNSQESVTEQDSKDSTYSLCSTLTLSKADYEKHKVYACEVTHQGLSSPVTKSFNRGES

SEQ ID NO: 68 - protein, 239 aa, synthetic construct
EVQLVESGGGLVQPGGSLRLSCATSGYTFTEYTMHWMRQAPGKGLEWVAGINPKNGGTSHNQRFMDRFTISVDKSTSTAYMQMNSLRAEDTAVYYCARWRGLNYGFDVRYFDVWGQGTLVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTCHHHHHH

SEQ ID NO: 69 - protein, 214 aa, synthetic construct
DIQMTQSPSSLSASVGDRVTITCRASQDINNYLNWYQQKPGKAPKLLIYYTSTLHSGVPSRFSGSGSGTDYTLTISSLQPEDFATYYCQQGNTLPPTFGQGTKVEIKRTVAAPSVFIFPPSDEQLKSGTASVVCLLNNFYPREAKVQWKVDNALQSGNSQESVTEQDSKDSTYSLCSTLTLSKADYEKHKVYACEVTHQGLSSPVTKSFNRGES

SEQ ID NO: 70 - protein, 237 aa, synthetic construct
QVQLQQSGPEVVKPGASVKMSCKASGYTFTSYVIHWVRQKPGQGLDWIGYINPYNDGTDYDEKFKGKATLTSDTSTSTAYMELSSLRSEDTAVYYCAREKDNYATGAWFAYWGQGTLVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTCHHHHHH

SEQ ID NO: 71 - protein, 481 aa, synthetic construct
DIVMTQSPDSLAVSLGERVTMNCKSSQSLLYSTNQKNYLAWYQQKPGQSPKLLIYWASTRESGVPDRFSGSGSGTDFTLTISSVQAEDVAVYYCQQYYSYRTFGGGTKLEIKRTVAAPSVFIFPPSDEQLKSGTASVVCLLNNFYPREAKVQWKVDNALQSGNSQESVTEQDSKDSTYSLCSTLTLSKADYEKHKVYACEVTHQGLSSPVTKSFNRGESGGGGSGGGGSGGGGSQVQLVESGGGVVQPGRSLRLSCAASGFTFSDFGMNWVRQAPGKGLEWVALIYYDGSNKFYADSVKGRFTISRDNSKNTLYLQMNSLRAEDTAVYYCAKPHYDGYYHFFDSWGQGTLVTVSSGGGGSGGGGSGGGGSGGGGSDIQMTQSPSSLSASVGDRVTITCKGSQDINNYLAWYQQKPGKAPKLLIYNTDILHTGVPSRFSGSGSGTDFTFTISSLQPEDIATYYCYQYNNGYTFGQGTKVEIK

SEQ ID NO: 72 - protein, 231 aa, synthetic construct; identical to SEQ ID NO: 10

SEQ ID NO: 73 - protein, 219 aa, synthetic construct; identical to SEQ ID NO: 11

SEQ ID NO: 74 - protein, 235 aa, synthetic construct
EVQLVESGGGLVQPGGSLRLSCAASGFTFSNYAMSWVRQAPGKRLEWVSAISDHSTNTYYPDSVKGRFTISRDNAKNTLYLQMNSLRAEDTAVYYCARKYGGDYDPFDYWGQGTLVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTCHHHHHH

SEQ ID NO: 75 - protein, 214 aa, synthetic construct
DIQMTQSPSSLSASVGDRVTITCQASQDINNYIAWYQHKPGKGPKLLIHYTSTLQPGIPSRFSGSGSGRDYTLTISSLQPEDFATYYCLQYDNLLFTFGGGTKVEIKRTVAAPSVFIFPPSDEQLKSGTASVVCLLNNFYPREAKVQWKVDNALQSGNSQESVTEQDSKDSTYSLCSTLTLSKADYEKHKVYACEVTHQGLSSPVTKSFNRGES

SEQ ID NO: 76 - protein, 136 aa, synthetic construct
EVQLVESGGGSVQPGGSLTLSCGTSGRTFNVMGWFRQAPGKEREFVAAVRWSSTGIYYTQYADSVKSRFTISRDNAKNTVYLEMNSLKPEDTAVYYCAADTYNSNPARWDGYDFRGQGTLVTVSSGGCGGHHHHHH

SEQ ID NO: 77 - protein, 132 aa, synthetic construct
EVQLVESGGGLVQAGGSLRLSCAASGSTFSDYGVGWFRQAPGKGREFVADIDWNGEHTSYADSVKGRFATSRDNAKNTAYLQMNSLKPEDTAVYYCAADALPYTVRKYNYWGQGTQVTVSSGGCGGHHHHHH

SEQ ID NO: 78 - protein, 138 aa, synthetic construct
EVQLVESGGGPVQAGGSLRLSCAASGRTYRGYSMGWFRQAPGKEREFVAAIVWSGGNTYYEDSVKGRFTISRDNAKNIMYLQMTSLKPEDSATYYCAAKIRPYIFKIAGQYDYWGQGTLVTVSSAGGGSGGHHHHHHC

SEQ ID NO: 79 - protein, 133 aa, synthetic construct
EVQLVESGGGLVQPGGSLRLSCAASGDIYKSFDMGWYRQAPGKQRDLVAVIGSRGNNRGRTNYADSVKGRFTISRDGTGNTVYLLMNKLRPEDTAIYYCNTAPLVAGRPWGRGTLVTVSSGGGSGGHHHHHHC

SEQ ID NO: 80 - protein, 138 aa, synthetic construct
DVQLQESGGGLVQAGGSLRLSCAVSGYPYSSYCMGWFRQAPGKEREGVAAIDSDGRTRYADSVKGRFTISQDNAKNTLYLQMNRMKPEDTAMYYCAARFGPMGCVDLSTLSFGHWGQGTQVTVSITGGGCHHHHHHHH

SEQ ID NO: 81 - protein, 128 aa, synthetic construct
EVQLVESGGGVVQPGGSLRLSCVASGYVHKINFYGWYRQAPGKEREKVAHISIGDQTDYADSAKGRFTISRDESKNTVYLQMNSLRPEDTAAYYCRALSRIWPYDYWGQGTLVTVSSGGCGGHHHHHH

SEQ ID NO: 82 - protein, 132 aa, synthetic construct
EVQLVESGGGLVQPGGSLRLSCAASGSIFRLHTMEWYRRTPETQREWVATITSGGTTNYPDSVKGRFTISRDDTKKTVYLQMNSLKPEDTAVYYCHAVATEDAGFPPSNYWGQGTLVTVSSGGCGGHHHHHH

SEQ ID NO: 83 - protein, 135 aa, synthetic construct
EVQLVESGGGPVQAGGSLRLSCAASGRTYRGYSMGWFRQAPGREREFVAAIVWSDGNTYYEDSVKGRFTISRDNAKNTMYLQMTSLKPEDSATYYCAAKIRPYIFKIAGQYDYWGQGTLVTVSSGGCGGHHHHHH

SEQ ID NO: 84 - protein, 235 aa, synthetic construct
EVKLVESGGGLVQPGRSLRLSCAASGFNFYAYWMGWVRQAPGKGLEWIGEIKKDGTTINYTPSLKDRFTISRDNAQNTLYLQMTKLGSEDTALYYCAREERDGYFDYWGQGVMVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTCGGHHHHHH

SEQ ID NO: 85 - protein, 215 aa, synthetic construct
QFVLTQPNSVSTNLGSTVKLSCKRSTGNIGSNYVNWYQQHEGRSPTTMIYRDDKRPDGVPDRFSGSIDRSSNSALLTINNVQTEDEADYFCQSYSSGIVFGGGTKLTVLSQPKAAPSVTLFPPSSEELQANKATLVCLVSDFYPGAVTVAWKADGSPVKVGVETTKPSKQSNNKYAACSYLSLTPEQWKSHRSYSCRVTHEGSTVEKTVAPAESS

SEQ ID NO: 86 - protein, 229 aa, synthetic construct
EVQLQQSGPELVKPGASVKMSCKASGYTFTSYVIQWVKQKPGQGLEWIGSINPYNDYTKYNEKFKGKATLTSDKSSITAYMEFSLTSEDSALYCARWGDGNYWGRGTLTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTCGGHHHHHH

SEQ ID NO: 87 - protein, 211 aa, synthetic construct
DIEMTQSPAIMSASLGERVTMTCTASSSVSSSYFHWYQKPGSSPKLCIYSTSNLASGVPPRFSGSGSTSYSLTISMEAEDAATYFCHQYHRSPTFGGGTKLETKRTVAAPSVFIFPPSDEQLKSGTASVVCLLNNFYPREAKVQWKVDNALQSGNSQESVTEQDSKDSTYSLCSTLTLSKADYEKHKVYACEVTHQGLSSPVTKSFNRGES

SEQ ID NO: 88 - protein, 238 aa, synthetic construct
QVQLKESGPGLVAPSQSLSITCTVSGFSLTGYGVNWVRQPPGKGLEWLGMIWGDGSTDYNSALKSRLSITKDNSKSQVFLKMNSLQTDDTARYYCARDGYSNFHYYVMDYWGQGTSVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTCGGHHHHHH

SEQ ID NO: 89 - protein, 219 aa, synthetic construct
DIVLTQSPASLAVSLGQRATISCRASESVEYYVTSLMQWYQQKPGQPPKLLISAASNVESGVPARFSGSGSGTDFSLNIHPVEEDDIAMYFCQQSRKVPWTFGGGTKLEIKRRTVAAPSVFIFPPSDEQLKSGTASVVCLLNNFYPREAKVQWKVDNALQSGNSQESVTEQDSKDSTYSLCSTLTLSKADYEKHKVYACEVTHQGLSSPVTKSFNRGES

SEQ ID NO: 90 - protein, 237 aa, synthetic construct
QVKLQQSGPGLVTPSQSLSITCTVSGFSLSDYGVHWVRQSPGQGLEWLGVIWAGGGTNYNSALMSRKSISKDNSKSQVFLKMNSLQADDTAVYYCARDKGYSYYYSMDYWGQGTTVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTCGGHHHHHH

SEQ ID NO: 91 - protein, 218 aa, synthetic construct
DIVLTQSPASLAVSLGQRATISCRASESVEYYVTSLMQWYQQKPGQPPKLLIFAASNVESGVPARFSGSGSGTNFSLNIHPVDEDDVAMYFCQQSRKVPYTFGGGTKLEIKRTVAAPSVFIFPPSDEQLKSGTASVVCLLNNFYPREAKVQWKVDNALQSGNSQESVTEQDSKDSTYSLCSTLTLSKADYEKHKVYACEVTHQGLSSPVTKSFNRGES

SEQ ID NO: 92 - protein, 234 aa, synthetic construct
QVQLQESGPGLVKPSETLSLTCAVSGFSLTSYGVHWIRQPGKGLEWLGVIWPGTNFNSALMSRLTISEDTSKNQVSLKLSSVTAADTAVYCARDRAYGNYLYAMDYWGQGTLVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTCGGHHHHHH

SEQ ID NO: 93 - protein, 210 aa, synthetic construct
DIQMTQSPSLSASVGDRVTITCRASESVEYVTSLMQWYQKPGKAPKLLIYAASNVDSGVPSRFSGSGTDFTLTISLQPEDIATYCQSRKVPFTFGGGTKVEIKRTVAAPSVFIFPPSDEQLKSGTASVVCLLNNFYPREAKVQWKVDNALQSGNSQESVTEQDSKDSTYSLCSTLTLSKADYEKHKVYACEVTHQGLSSPVTKSFNRGES

SEQ ID NO: 94 - protein, 233 aa, synthetic construct
QVQLVQSGAEVKKPGASVKVSCKASGYTFTDYKIHWVRQAPGQGLEWIGYIYPYSGSSDYNQKFKSRATLTVDNSISTAYMELSRLRSDDTAVYYCARGGDAMDYWGQGTLVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTCGGHHHHHH

SEQ ID NO: 95 - protein, 213 aa, synthetic construct
DIQMTQSPSSLSASVGDRVTITCGASENIYGALNWYQRKPGKAPKLLIYGATNLADGVPSRFSGSGSGRDYTLTISSLQPEDFATYFCQNILGTWTFGGGTKVEIKRTVAAPSVFIFPPSDEQLKSGTASVVCLLNNFYPREAKVQWKVDNALQSGNSQESVTEQDSKDSTYSLCSTLTLSKADYEKHKVYACEVTHQGLSSPVTKSFNRGES

SEQ ID NO: 96 - protein, 234 aa, synthetic construct
EVQLVESGGGLVQPGGSLRLSCAASGFTFNIYYMSWVRQAPGKGLELVAAINPDGGNTYYPDTVKGRFTISRDNAKNSLYLQMNSLRAEDTAVYYCARYGGPGFDSWGQGTLVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTCGGHHHHHH

SEQ ID NO: 97 - protein, 215 aa, synthetic construct
ENVLTQSPATLSLSPGERATLSCSASSSVSYMHWYQQKPGQAPRLWIYDTSKLASGIPARFSGSGSRNDYTLTISSLEPEDFAVYYCFPGSGFPFMYTFGGGTKVEIKRTVAAPSVFIFPPSDEQLKSGTASVVCLLNNFYPREAKVQWKVDNALQSGNSQESVTEQDSKDSTYSLCSTLTLSKADYEKHKVYACEVTHQGLSSPVTKSFNRGES

SEQ ID NO: 98 - protein, 262 aa, synthetic construct
QVQLVESGGGVVQPGRSLRLSCAASGFTFSDFGMNWVRQAPGKGLEWVALIYYDGSNKFYADSVKGRFTISRDNSKNTLYLQMNSLRAEDTAVYYCAKPHYDGYYHFFDSWGQGTLVTVSSGGGGSGGGGSGGGGSGGGGSDIQMTQSPSSLSASVGDRVTITCKGSQDINNYLAWYQQKPGKAPKLLIYNTDILHTGVPSRFSGSGSGTDFTFTISSLQPEDIATYYCYQYNNGYTFGQGTKVEIKGGGSGGCGGHHHHHH

SEQ ID NO: 99 - protein, 243 aa, synthetic construct
DVQLQESGGGLVQAGGSLRLSCAVSGYPYSSYCMGWFRQAPGKEREGVAAIDSDGRTRYADSVKGRFTISQDNAKNTLYLQMNRMKPEDTAMYYCAARFGPMGCVDLSTLSFGHWGQGTQVTVSITASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTCGGHHHHHH

SEQ ID NO: 100 - protein, 8 aa, synthetic construct
GSTFSDYG

SEQ ID NO: 101 - protein, 8 aa, synthetic construct
IDWNGEHT

SEQ ID NO: 102 - protein, 14 aa, synthetic construct
AADALPYTVRKYNY

SEQ ID NO: 103 - protein, 4 aa, synthetic construct
KERE

SEQ ID NO: 104 - protein, 4 aa, synthetic construct
KQRE

SEQ ID NO: 105 - protein, 4 aa, synthetic construct
GLEW

SEQ ID NO: 106 - protein, 5 aa, synthetic construct
KEREL

SEQ ID NO: 107 - protein, 5 aa, synthetic construct
KEREF

SEQ ID NO: 108 - protein, 5 aa, synthetic construct
KQREL

SEQ ID NO: 109 - protein, 5 aa, synthetic construct
KQREF

SEQ ID NO: 110 - protein, 5 aa, synthetic construct
KEREG

SEQ ID NO: 111 - protein, 5 aa, synthetic construct
KQREW

SEQ ID NO: 112 - protein, 5 aa, synthetic construct
KQREG

SEQ ID NO: 113 - protein, 4 aa, synthetic construct
TERE

SEQ ID NO: 114 - protein, 5 aa, synthetic construct
TEREL

SEQ ID NO: 115 - protein, 4 aa, synthetic construct
TQRE

SEQ ID NO: 116 - protein, 5 aa, synthetic construct
TQREL

SEQ ID NO: 117 - protein, 4 aa, synthetic construct
KECE

SEQ ID NO: 118 - protein, 5 aa, synthetic construct
KECEL

SEQ ID NO: 119 - protein, 5 aa, synthetic construct
KECER

SEQ ID NO: 120 - protein, 4 aa, synthetic construct
KQCE

SEQ ID NO: 121 - protein, 5 aa, synthetic construct
KQCEL

SEQ ID NO: 122 - protein, 4 aa, synthetic construct
RERE

SEQ ID NO: 123 - protein, 5 aa, synthetic construct
REREG

SEQ ID NO: 124 - protein, 4 aa, synthetic construct
RQRE

SEQ ID NO: 125 - protein, 5 aa, synthetic construct
RQREL

SEQ ID NO: 126 - protein, 5 aa, synthetic construct
RQREF

SEQ ID NO: 127 - protein, 5 aa, synthetic construct
RQREW

SEQ ID NO: 128 - protein, 4 aa, synthetic construct
QERE

SEQ ID NO: 129 - protein, 5 aa, synthetic construct
QEREG

SEQ ID NO: 130 - protein, 4 aa, synthetic construct
QQRE

SEQ ID NO: 131 - protein, 5 aa, synthetic construct
QQREW

SEQ ID NO: 132 - protein, 5 aa, synthetic construct
QQREL

SEQ ID NO: 133 - protein, 5 aa, synthetic construct
QQREF

SEQ ID NO: 134 - protein, 4 aa, synthetic construct
KGRE

SEQ ID NO: 135 - protein, 5 aa, synthetic construct
KGREG

SEQ ID NO: 136 - protein, 4 aa, synthetic construct
KDRE

SEQ ID NO: 137 - protein, 5 aa, synthetic construct
KDREV

SEQ ID NO: 138 - protein, 5 aa, synthetic construct
DECKL

SEQ ID NO: 139 - protein, 5 aa, synthetic construct
NVCEL

SEQ ID NO: 140 - protein, 4 aa, synthetic construct
GVEW

SEQ ID NO: 141 - protein, 4 aa, synthetic construct
EPEW

SEQ ID NO: 142 - protein, 4 aa, synthetic construct
GLER

SEQ ID NO: 143 - protein, 4 aa, synthetic construct
DQEW

SEQ ID NO: 144 - protein, 4 aa, synthetic construct
DLEW

SEQ ID NO: 145 - protein, 4 aa, synthetic construct
GIEW

SEQ ID NO: 146 - protein, 4 aa, synthetic construct
ELEW

SEQ ID NO: 147 - protein, 4 aa, synthetic construct
GPEW

SEQ ID NO: 148 - protein, 4 aa, synthetic construct
EWLP

SEQ ID NO: 149 - protein, 4 aa, synthetic construct
GPER

SEQ ID NO: 150-151 - skipped (2 entries)

SEQ ID NO: 152 - protein, 5 aa, synthetic construct
  VARIANT 1..5: note = May be present in repeats of 1, 2, 3, 4, 5, 6, 7 or more
GGGGS

SEQ ID NO: 153 - skipped

SEQ ID NO: 154 - protein, 5 aa, synthetic construct
GGGGS

SEQ ID NO: 155 - protein, 7 aa, synthetic construct
SGGSGGS

SEQ ID NO: 156 - protein, 8 aa, synthetic construct
GGGGSGGS

SEQ ID NO: 157 - protein, 9 aa, synthetic construct
GGGGSGGGS

SEQ ID NO: 158 - protein, 10 aa, synthetic construct
GGGGSGGGGS

SEQ ID NO: 159 - protein, 15 aa, synthetic construct
GGGGSGGGGSGGGGS

SEQ ID NO: 160 - protein, 18 aa, synthetic construct
GGGGSGGGGSGGGGSGGS

SEQ ID NO: 161 - protein, 20 aa, synthetic construct
GGGGSGGGGSGGGGSGGGGS

SEQ ID NO: 162 - protein, 25 aa, synthetic construct
GGGGSGGGGSGGGGSGGGGSGGGGS

SEQ ID NO: 163 - protein, 30 aa, synthetic construct
GGGGSGGGGSGGGGSGGGGSGGGGSGGGGS

SEQ ID NO: 164 - protein, 35 aa, synthetic construct
GGGGSGGGGSGGGGSGGGGSGGGGSGGGGSGGGGS

SEQ ID NO: 165 - protein, 40 aa, synthetic construct
GGGGSGGGGSGGGGSGGGGSGGGGSGGGGSGGGGSGGGGS

SEQ ID NO: 166 - protein, 15 aa, synthetic construct
EPKSCDKTHTCPPCP

SEQ ID NO: 167 - protein, 24 aa, synthetic construct
GGGGSGGGSEPKSCDKTHTCPPCP

SEQ ID NO: 168 - protein, 12 aa, synthetic construct
EPKTPKPQPAAA

SEQ ID NO: 169 - protein, 62 aa, synthetic construct
ELKTPLGDTTHTCPRCPEPKSCDTPPPCPRCPEPKSCDTPPPCPRCPEPKSCDTPPPCPRCP

(19) United States
(12) Patent Application Publication
(10) Pub. No.: US 2024/0002331 A1
(43) Pub. Date: Jan. 4, 2024

(54) IONIZABLE CATIONIC LIPIDS AND LIPID NANOPARTICLES, AND METHODS OF SYNTHESIS AND USE THEREOF

(71) Applicant: Tidal Therapeutics, Inc., Cambridge, MA (US)

(72) Inventors: Mir ALI, Woburn, MA (US); Austin Wayne BOESCH, Somerville, MA (US); Daryl Clark DRUMMOND, Lincoln, MA (US); William KUHLMAN, Somerville, MA (US); Ulrik NIELSEN, Quincy, MA (US)

(21) Appl. No.: 18/331,046

(22) Filed: Jun. 7, 2023

Related U.S. Application Data
(60) Provisional application No. 63/350,404, filed on Jun. 8, 2022.

(51) Int. Cl. C07C 237/16; A61K 47/69; C07K 16/28; A61P 37/02

(52) U.S. Cl. CPC C07C 237/16; A61K 47/6929; C07K 16/2803; A61P 37/02

(57) ABSTRACT

Provided are ionizable cationic lipids and lipid nanoparticles for the delivery of nucleic acids to cells (e.g., immune cells), and methods of making and using such lipids and targeted lipid nanoparticles.

### CROSS-REFERENCE TO RELATED APPLICATIONS

This application claims priority to and benefit of U.S. Provisional Application No. 63/350,404, filed Jun. 8, 2022, the disclosure of which is hereby incorporated herein by reference in its entirety.

### REFERENCE TO AN ELECTRONIC SEQUENCE LISTING

The contents of the electronic sequence listing (183952034200seqlist.xml; Size: 168,364 bytes; and Date of Creation: Jun. 1, 2023) are herein incorporated by reference in their entirety.

### FIELD OF THE INVENTION

The invention provides ionizable cationic lipids and lipid nanoparticles for the delivery of nucleic acids to immune cells, and methods of making and using, such lipids and targeted lipid nanoparticles.

### BACKGROUND

In recent years, a number of therapeutic modalities have been developed that involve the delivery of one or more nucleic acids to a subject. Treatment modalities include, for example, gene therapies where a gene of interest in the form of deoxyribose nucleic acid (DNA) is introduced into a cell, which is then expressed to produce a gene product, for example, protein, for treating a disorder caused by or associated with a deficiency or absence of the gene product. In this approach, the gene is transcribed into a messenger ribonucleic acid (mRNA), whereupon the mRNA is translated to produce the gene product. In another approach, mRNA rather than a gene of interest can be delivered to the cell. The resulting expression product can ameliorate the deficiency or absence of a particular protein in a subject (for example, a protein deficiency occurring in certain forms of cystic fibrosis or lysosomal storage disorders), or can be used to modulate a cellular function, for example, reprogramming immune cells to initiate or otherwise modulate an immune response in the subject (for example, as a therapeutic agent for treating cancer or as a prophylactic vaccine for preventing or minimizing the risk or severity of a microbial or viral infection).

However, the delivery of mRNA to a cell for translation within the cell has been challenging for a variety of factors, such as nuclease degradation of the mRNA prior to entry into the cell and then after introduction into the cell but prior to translation.

RNA may be delivered to a subject using different delivery vehicles, for example, based on cationic polymers or lipids which, together with the RNA, form nanoparticles. The nanoparticles are intended to protect the RNA from degradation, enable delivery of the RNA to the target site and facilitate cellular uptake and processing by the target cells. For delivery efficacy, in addition to the molecular composition, parameters like particle size, charge, or grafting with molecular moieties, such as polyethylene glycol (PEG) or ligands, play a role. Grafting with PEG is believed to reduce serum interactions, increase serum stability and increase time in circulation, which can be helpful for certain targeting approaches.

Compared with DNA delivery technologies used in certain gene therapies, mRNA-based gene treatment has a number of superior features, for example, ease in manipulation, rapid and transient expression, and adaptive convertibility without mutagenesis.

However, the delivery of therapeutic RNAs to cells is difficult in view of the relative instability and low cell permeability of RNAs. Thus, there exists a need to develop methods and compositions to facilitate the delivery of RNAs such as mRNA to cells.

### SUMMARY

The invention provides ionizable cationic lipids, lipid-immune cell targeting group conjugates, and lipid nanoparticle compositions comprising such ionizable cationic lipids and/or lipid-immune cell (e.g., T-cell) targeting group conjugates, medical kits containing such lipids and/or conjugates, and methods of making and using, such lipids and conjugates.

The lipid nanoparticle compositions provided herein may further comprise a nucleic acid, such as an RNA, e.g., a messenger RNA or mRNA. The lipid nanoparticle compositions may be used for mRNA delivery to a cell (e.g., an immune cell, such as T-cell) in a subject. Messenger RNA based gene therapy requires efficient delivery of mRNA to circulating cells (e.g., immune cells, such as T-cells or NK cells) in plasma or to cells in a given tissue. The main challenges associated with efficient mRNA delivery to attain robust levels of protein expression include: (a) ability to protect the mRNA payload against prevalent serum nucleases upon administration to a subject; (b) the ability to specifically target mRNA delivery to, and thereby maximize protein expression in the target cell (e.g., T-cell) population; and (c) the ability to maximally deliver the mRNA payload to the cytosolic compartment of cells (e.g., T-cells) for translation into proteins within the cytoplasm.

The invention provides ionizable cationic lipids for producing lipid nanoparticle compositions that facilitate the delivery of a payload (e.g., a nucleic acid, such as a DNA or RNA, such as an mRNA) disposed therein to cells, for example, mammalian cells, for example, immune cells. The lipids are designed to enable intracellular delivery of a nucleic acid, e.g., mRNA, to the cytosolic compartment of a target cell type and rapidly degrade into non-toxic components. These complex functionalities are achieved by the interplay between chemistry and geometry of the ionizable lipid head group, the hydrophobic “acyl-tail” groups and the linker connecting the head group and the acyl tail groups in the ionizable cationic lipids.

In one aspect, the present invention provides a compound represented by Formula (I):

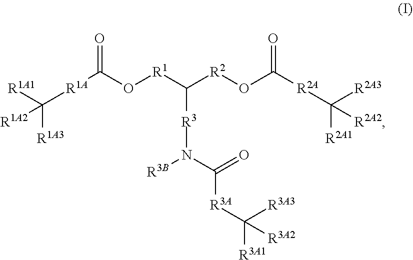

or a salt thereof. In some embodiments, R¹, R², and R³ are each independently a bond or C₁₋₃ alkylene. In some embodiments, R^{1A}, R^{2A}, and R^{3A} are each independently a bond or C₁₋₁₀ alkylene. In some embodiments, R^{1A1}, R^{1A2}, R^{1A3}, R^{2A}, R^{2A2}, R^{2A3}, R^{3A1}, R^{3A2}, and R^{3A3} are each independently H, C₁₋₂₀ alkyl, C₁₋₂₀ alkenyl, —(CH₂)₀₋₁₀C(O)OR^{a1}, or —(CH₂)₀₋₁₀OC(O)R^{a2}. In some embodiments, R^{a1} and R^{a2} are each independently C₁₋₂₀ alkyl or C₁₋₂₀ alkenyl. In some embodiments, R^{3B} is

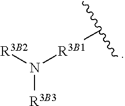

In some embodiments, R^{3B1} is C₁₋₆ alkylene. In some embodiments, R^{3B2} and R^{3B3} are each independently H or C₁₋₆ alkyl. In some embodiments, R^{3B2} and R^{3B3} are each independently H, unsubstituted C₁₋₆ alkyl, or C₁₋₆ alkyl substituted with one or more substituents each independently selected from the group consisting of —OH and —O—(C₁₋₆ alkyl).

Also provided herein is a lipid nanoparticle (LNP) comprising a lipid blend comprising an ionizable cationic lipid and/or lipid-immune cell targeting group conjugate (e.g., a lipid-T-cell targeting group conjugate) provided herein.

In another aspect, provided herein is a method of delivering a nucleic acid to an immune cell (e.g., a T-cell), the method comprising exposing the immune cell to an LNP described herein containing the nucleic acid under conditions that permit the nucleic acid to enter the immune cell.

In another aspect, provided herein is a method of delivering a nucleic acid to an immune cell (e.g., a T-cell) in a subject in need thereof, the method comprising administering to the subject a composition comprising an LNP described herein containing a nucleic acid thereby to deliver the nucleic acid to the immune cell.

In another aspect, provided herein is a method of targeting the delivering of a nucleic acid (e.g., mRNA) to an immune cell (e.g., a T-cell) in a subject, the method comprising administering to the subject an LNP described herein containing the nucleic acid so as to facilitate targeted delivery of the nucleic acid to the immune cell.

In one aspect, provided herein are lipid nanoparticles (LNPs) comprising a lipid blend for targeted delivery of a nucleic acid into an immune cell. In some embodiments, the lipid blend comprises a lipid-immune cell targeting group conjugate comprising the compound of Formula (II): [Lipid]-[optional linker]-[immune cell targeting group]. In some embodiments, the lipid blend comprises an ionizable cationic lipid. In some embodiments, the ionizable cationic lipid comprises an ionizable cationic lipid as described herein. In some embodiments, the LNP comprises a nucleic acid disposed therein.

In some embodiments, the immune cell targeting group comprises an antibody that binds a T cell antigen. In some embodiments, the T cell antigen is CD3, CD4, CD7, or CD8, or a combination thereof (e.g., both CD3 and CD8, both CD4 and CD8, or both CD7 and CD8). In some embodiments, the immune cell targeting group comprises an antibody that binds a Natural Killer (NK) cell antigen. In some embodiments, the NK cell antigen is CD7, CD8, or CD56, or a combination thereof (e.g., both CD7 and CD8). In some embodiments, the antibody is a human or humanized antibody.

In some embodiments, the immune cell targeting group is covalently coupled to a lipid in the lipid blend via a polyethylene glycol (PEG) containing linker. In some embodiments, the lipid covalently coupled to the immune cell targeting group via a PEG containing linker is distearoylglycerol (DSG), distearoyl-phosphatidylethanolamine (DSPE), dimyrstoyl-phosphatidylethanolamine (DMPE), distearoyl-glycero-phosphoglycerol (DSPG), dimyristoyl-glycerol (DMG), dipalmitoyl-phosphatidylethanolamine (DPPE), dipalmitoyl-glycerol (DPG), or ceramide. In some embodiments, the PEG is PEG 2000.

In some embodiments, the lipid-immune cell targeting group conjugate is present in the lipid blend in a range of 0.002-0.2 mole percent. In some embodiments, the lipid blend comprises one or more of a structural lipid (e.g., a sterol), a neutral phospholipid, and a free PEG-lipid. In some embodiments, the ionizable cationic lipid is present in the lipid blend in a range of 40-60 mole percent. In some embodiments, the sterol is present in the lipid blend in a range of 30-50 mole percent. In some embodiments, the sterol is present in the lipid blend in a range of 20-70 mole percent. In some embodiments, the sterol is cholesterol.

In some embodiments, the neutral phospholipid is selected from the group consisting of phosphatidylcholine, phosphatidylethanolamine, distearoyl-sn-glycero-3-phosphoethanolamine (DSPE), 1,2-distearoyl-sn-glycero-3-phosphocholine (DSPC), hydrogenated soy phosphatidylcholine (HSPC), 1,2-dioleoyl-sn-glycero-3-phosphoethanolamine (DOPE), 1,2-dioleoyl-sn-glycero-3-phosphocholine (DOPC), sphingomyelin (SM). In some embodiments, the neutral phospholipid is present in the lipid blend in a range of 1-15 mole percent, such as about 5-15 mole percent.

In some embodiments, the free PEG-lipid is selected from the group consisting of PEG-distearoyl-phosphatidylethanolamine (PEG-DSPE) or PEG-dimyrstoyl-phosphatidylethanolamine (PEG-DMPE), N-(Methylpolyoxyethylene oxycarbonyl)-1,2-dipalmitoyl-sn-glycero-3-phosphoethanolamine (DPPE-PEG) 1,2-Dimyristoyl-rac-glycero-3-methylpolyoxyethylene (PEG-DMG), 1,2-Dipalmitoyl-rac-glycero-3-methylpolyoxyethylene (PEG-DPG), 1,2-Dioleoyl-rac-glycerol, methoxypolyethylene Glycol (DOG-PEG) 1,2-Distearoyl-rac-glycero-3-methylpolyoxyethylene (PEG-DSG), N-palmitoyl-sphingosine-1-{succinyl[methoxy(polyethylene glycol)] (PEG-ceramide), and DSPE-PEG-cysteine, or a derivative thereof. In some embodiments, the free PEG-lipid comprises a diacylphosphatidylethanolamines comprising Dipalmitoyl (C16) chain or Distearoyl (C18) chain. In some embodiments the free PEG-lipid is a mixture of two or more unique free PEG-lipids. In some embodiments, the free PEG-lipid is present in the lipid blend in a range of 1-4 mole percent, such as about 1-2 mole percent, or about 2-4 mole percent, or about 1.5 mole percent. In some embodiments, the free PEG-lipid comprises the same or a different lipid as the lipid in the lipid-immune cell targeting group conjugate.

In some embodiments, the LNP has a mean diameter in the range of 50-200 nm. In some embodiments, the LNP has a mean diameter of about 100 nm. In some embodiments, the LNP has a polydispersity index in a range from 0.05 to 1. In some embodiments, the LNP has a zeta potential of from about +5 mV to about +50 mV at pH5, such as about +10 mV to about +30 mV at pH 5. In some embodiments, the LNP has a zeta potential of from about −10 mV to about +10 mV at pH 7.4.

In some embodiments, the nucleic acid is DNA or RNA. In some embodiments, the RNA is an mRNA, tRNA, siRNA, gRNA (guide RNA), circRNA (circular RNA), ribozymes, decoy RNA, or microRNA. In some embodiments, the mRNA encodes a receptor, a growth factor, a hormone, a cytokine, an antibody, an antigen, an enzyme, or a vaccine. In some embodiments, the mRNA encodes a polypeptide capable of regulating immune response in the immune cell. In some embodiments, the mRNA encodes a polypeptide capable of reprogramming the immune cell. In some embodiments, the mRNA encodes a synthetic T cell receptor (synTCR) or a Chimeric Antigen Receptor (CAR). In some embodiments, the CAR is TTR-023 anti-CD20 (Leu-16). In some embodiments, the CAR comprises the amino acid sequence of SEQ ID NO: 24. In some embodiments, the mRNA encoding the CAR comprises the polynucleotide sequence of 25. TTR-023 anti-CD20 (Leu-16) CAR sequence (including leader) (SEQ ID NO: 24):

(SEQ ID NO: 24)
METDTLLLWVLLLWVPGSTGDYKAKEVQLQQSGAELVKPGASVKMSCKA
SGYTFTSYNMHWVKQTPGQGLEWIGAIYPGNGDTSYNQKFKGKATLTAD
KSSSTAYMQLSSLTSEDSADYYCARSNYYGSSYWFFDVWGAGTTVTVSS
GGGSGGGSGGGGSSDIVLTQSPAILSASPGEKVTMTCRASSSVNYMDWY
QKKPGSSPKPWIYATSNLASGVPARFSGSGSGTSYSLTISRVEAEDAAT
YYCQQWSFNPPTFGGGTKLEIKGGGGSAAAIEVMYPPPYLDNEKSNGTI
IHVKGKHLCPSPLFPGPSKPFWVLVVVGGVLACYSLLVTVAFIIFWVRS
KRSRLLHSDYMNMTPRRPGPTRKHYQPYAPPRDFAAYRSRVKFSRSAEP
PAYQQGQNQLYNELNLGRREEYDVLDKRRGRDPEMGGKPRRKNPQEGLY
NELQKDKMAEAYSEIGMKGERRRGKGHDGLYQGLSTATKDTYDALHMQA
LPPR
Corresponding nucleic acid sequence
(SEQ ID NO: 25):
(SEQ ID NO: 25)
atggagaccgacaccctgttgctttgggtactgttactttgggtgcccg
gatctaccggtgattacaaggccaaggaggtgcagctgcagcagagcgg
agccgagctggtgaagccaggcgcttccgtgaagatgtcttgtaaggcc
tccggctacacattcaccagctacaatatgcactgggtaaagcagactc
cggggcagggcctggagtggataggtgccatctaccctggcaacggcga
caccagctacaaccagaagtttaaggggaaggctactctaacagcggac
aagtcgtcctctaccgcctacatgcaactcagctccctgacgagcgagg
actccgcggactactactgtgcccgctccaactactacggctctagcta
ttggttcttcgacgtgtggggcgctggaacgaccgtgaccgtgtcttcc
ggtggaggttccgggggcggaagcggcggtggcggcagttcggacatcg
tgctgacccagagccctgccatcctgtccgcttccccgggggagaaagt
tacgatgacctgccgagcgagctccagtgtcaactacatggattggtac
cagaagaagcccggcagcagtcccaagccgtggatttacgctactagca
acctggcgtccggtgtcccggctcgcttctcaggttctggctcgggtac
tagttattcattaaccatttctcgcgtggaggctgaggacgctgccacc
tactactgccaacagtggtctttcaaccctcccactttcggaggcggca
ccaagctcgagatcaaggggggggtggctccgcagcagccattgaggtg
atgtatcctcctccctatttggacaacgagaagtcaaatggcaccatca
tccacgttaagggcaagcacctgtgcccatctcccctgttcccaggccc
ctctaagcccttctgggtcctggtggtggtcggcggcgtcctggcatgt
tactctctgctggtgaccgtcgcgttcatcatcttttgggtccggtcca
agcgcagccgcctgctccactccgactacatgaatatgactcctcgtag
gcccggtccaacccgcaagcactaccagccgtacgcgccgcccagagac
tttgctgcttaccgatccagagtgaaattttctaggtcggccgaacctc
ccgcatatcagcagggccagaaccagctgtacaacgaactcaacttggg
acggcgcgaggaatacgatgtgctggataaacgccgtggccgcgatccc
gagatgggcgggaagccacgtcgcaaaaaccctcaggagggcctttaca
acgagttgcagaaggacaaaatggcggaggcctactccgagatcggaat
gaagggggagcgccggcgcggcaaagggcatgacggcctctaccagggc
ctgtccacagccacgaaagacacctatgacgccctgcatatgcaggccc
tgcccccgcgctgataatga

In some embodiments, the immune cell targeting group comprises an antibody, and the antibody is a Fab or an immunoglobulin single variable domain, such as a Nanobody. In some embodiments, the immune cell targeting group comprises a Fab, F(ab′)2, Fab′-SH, Fv, or scFv fragment. In some embodiments, the immune cell targeting group comprises a Fab that is engineered to knock out one or more natural interchain disulfide bonds. For example, in some embodiments, the Fab comprises a heavy chain fragment that comprises C233S substitution, numbering according to Kabat, and/or a light chain fragment that comprises C214S substitution, numbering according to Kabat. In some embodiments, the immune cell targeting group comprises a Fab that is engineered to introduce one or more buried interchain disulfide bonds. For example, in some embodiments, the Fab antibody comprises a heavy chain fragment that comprises F174C substitution, numbering according to Kabat, and/or a light chain fragment that comprises S176C substitution, numbering according to Kabat. In some embodiments, the immune cell targeting group comprises a Fab that is engineered to knock out one or more natural interchain disulfide bonds, and to introduce one or more buried interchain disulfide bonds. In some embodiments, the immune cell targeting group comprises a Fab that comprises a cysteine at the C-terminus of the heavy or light chain fragment. In some embodiments, the Fab further comprises one or more amino acids between the heavy chain fragment of the Fab and the C-terminal cysteine. In some embodiments, the Fab comprises a heavy chain variable domain linked to an antibody CH1 domain and a light chain variable domain linked to an antibody light chain constant domain, wherein the CH1 domain and the light chain constant domain are linked by one or more interchain disulfide bonds, and wherein the immune cell targeting group further comprises a single chain variable fragment (scFv) linked to the C-terminus of the light chain constant domain by an amino acid linker. In some embodiments, the Fab antibody is a DS Fab (Fab with wild type (natural) interchain disulfide bond), a NoDS Fab (Fab with natural disulfide bond knocked out, such as a Fab with C233S substitution on the heavy chain, and/or C214S substitution on the light chain, numbering according to Kabat), a bDS Fab (Fab without natural disulfide bond, and with introduced non-natural interchain buried disulfide bond, such as a Fab with F174C and C233S on the heavy chain, and/or S176C and C214S substitution on the light chain, numbering according to Kabat), or a bDS Fab-ScFv (a bDS Fab linked to a ScFv through a linker, such as (G4S)x), as demonstrated in FIG. 31.

In some embodiments, the immune cell targeting group comprises an immunoglobulin single variable domain, such as a Nanobody. In some embodiments, the immunoglobulin single variable domain comprises a cysteine at the C-terminus. In some embodiments, the Nanobody further comprises a spacer comprising one or more amino acids between the V_{HH} domain and the C-terminal cysteine. In some embodiments, the immune cell targeting group comprises two or more V_{HH} domains. In some embodiments, the two or more V_{HH} domains are linked by an amino acid linker. In some embodiments, the immune cell targeting group comprises a first V_{HH} domain linked to an antibody CH1 domain and a second V_{HH} domain linked to an antibody light chain constant domain, and wherein the antibody CH1 domain and the antibody light chain constant domain are linked by one or more disulfide bonds. In some embodiments, the immune cell targeting group comprises a V_{HH} domain linked to an antibody CH1 domain, and wherein the antibody CH1 domain is linked to an antibody light chain constant domain by one or more disulfide bonds. In some embodiments, the CH1 domain comprises F174C and C233S substitutions, and/or the light chain constant domain comprises S176C and C214S substitutions, numbering according to Kabat. In some embodiments, the antibody is a ScFv, a V_{HH} (Nb), a 2×V_{HH}, a V_{HH}-CH1/empty Vk, or a V_{HH}1-CH1/V_{HH}-2-Nb bDS, as demonstrated in FIG. 31.

In some embodiments, the immune cell targeting group comprises a Fab that comprises a heavy chain fragment comprising the amino acid sequence of SEQ ID NO: 1 and a light chain fragment comprising the amino acid sequence of SEQ ID NO: 2 or 3. In some embodiments, the immune cell targeting group comprises a Fab that comprises a heavy chain fragment comprising the amino acid sequence of SEQ ID NO: 6 and a light chain fragment comprising the amino acid sequence of SEQ ID NO: 7. In some embodiments, the antibody is an antibody described in the examples.

In some embodiments, the immune cell targeting group comprises a Fab that comprises:
(a) a heavy chain fragment comprising the amino acid sequence of SEQ ID NO: 1 and a light chain fragment comprising the amino acid sequence of SEQ ID NO: 2 or 3;
(b) a heavy chain fragment comprising the amino acid sequence of SEQ ID NO: 4 and a light chain fragment comprising the amino acid sequence of SEQ ID NO: 5;
(c) a heavy chain fragment comprising the amino acid sequence of SEQ ID NO: 6 and a light chain fragment comprising the amino acid sequence of SEQ ID NO: 7;
(d) a heavy chain fragment comprising the amino acid sequence of SEQ ID NO: 8 and a light chain fragment comprising the amino acid sequence of SEQ ID NO: 9;
(e) a heavy chain fragment comprising the amino acid sequence of SEQ ID NO: 10 and a light chain fragment comprising the amino acid sequence of SEQ ID NO: 11;
(f) a heavy chain fragment comprising the amino acid sequence of SEQ ID NO: 12 and a light chain fragment comprising the amino acid sequence of SEQ ID NO: 13;
(g) a heavy chain fragment comprising the amino acid sequence of SEQ ID NO: 14 and a light chain fragment comprising the amino acid sequence of SEQ ID NO: 15;
(h) a heavy chain fragment comprising the amino acid sequence of SEQ ID NO: 16 and a light chain fragment comprising the amino acid sequence of SEQ ID NO: 17;
(i) a heavy chain fragment comprising the amino acid sequence of SEQ ID NO: 18 and a light chain fragment comprising the amino acid sequence of SEQ ID NO: 19;
(j) a heavy chain fragment comprising the amino acid sequence of SEQ ID NO: 20 and a light chain fragment comprising the amino acid sequence of SEQ ID NO: 21; or
(k) a heavy chain fragment comprising the amino acid sequence of SEQ ID NO: 22 and a light chain fragment comprising the amino acid sequence of SEQ ID NO: 23.

In some embodiments, the immune cell targeting group comprises a Fab, F(ab′)2, Fab′-SH, Fv, or scFv fragment. In some embodiments, the immune cell targeting group comprises a Fab that is engineered to knock out the natural interchain disulfide bond at the C-terminus. In some embodiments, the Fab comprises a heavy chain fragment that comprises C233S substitution, and a light chain fragment that comprises C214S substitution, numbering according to Kabat. In some embodiments, the immune cell targeting group comprises a Fab that has a non-natural interchain disulfide bond (e.g., an engineered, buried interchain disulfide bond). In some embodiments, the Fab comprises F174C substitution in the heavy chain fragment, and S176C substitution in the light chain fragment, numbering according to Kabat. In some embodiments, the immune cell targeting group comprises a Fab that comprises a cysteine at the C-terminus of the heavy or light chain fragment. In some embodiments, the Fab further comprises one or more amino acids between the heavy chain fragment of the Fab and the C-terminal cysteine.

In some embodiments, the immune cell targeting group comprises an immunoglobulin single variable domain. In some embodiments, the immunoglobulin single variable domain comprises a cysteine at the C-terminus. In some embodiments, the immunoglobulin single variable domain comprises a VHH domain and further comprises a spacer comprising one or more amino acids between the VHH domain and the C-terminal cysteine. In some embodiments, the immune cell targeting group comprises two or more VHH domains. In some embodiments, the two or more V_{HH} domains are linked by an amino acid linker. In some embodiments, the immune cell targeting group comprises a first V_{HH} domain linked to an antibody CH1 domain and a second V_{HH} domain linked to an antibody light chain constant domain. In some embodiments, the antibody CH1 domain and the antibody light chain constant domain are linked by one or more disulfide bonds. In some embodiments, the immune cell targeting group comprises a VHH domain linked to an antibody CH1 domain. In some embodiments, the antibody CH1 domain is linked to an antibody light chain constant domain by one or more disulfide bonds. In some embodiments, the CH1 domain comprises F174C and C233S substitutions, and the light chain constant domain comprises S176C and C214S substitutions, numbering according to Kabat.

In some embodiments, the immune cell targeting group comprises a Fab that comprises: (a) a heavy chain fragment comprising the amino acid sequence of SEQ ID NO: 1 and a light chain fragment comprising the amino acid sequence of SEQ ID NO: 2 or 3; or (b) a heavy chain fragment comprising the amino acid sequence of SEQ ID NO: 6 and a light chain fragment comprising the amino acid sequence of SEQ ID NO: 7.

In another aspect, provided herein are methods of targeting the delivery of a nucleic acid to an immune cell of a subject. In some embodiments, the method comprises contacting the immune cell with a lipid nanoparticle (LNP). In some embodiments, the LNP comprises an ionizable cationic lipid. In some embodiments, the LNP comprises a conjugate comprising the compound of the following formula (II): [Lipid]-[optional linker]-[immune cell targeting group]. In some embodiments, the LNP comprises a sterol or other structural lipid. In some embodiments, the LNP comprises a neutral phospholipid. In some embodiments, the LNP comprises a free Polyethylene glycol (PEG) lipid. In some embodiments, the LNP comprises the nucleic acid.

In some embodiments, an aspect of the disclosure relates to an LNP or a pharmaceutical composition containing thereof, as disclosed herein, for use in a method of targeting the delivery of a nucleic acid to an immune cell of a subject. Such a method may be for the treatment of a disease or disorder as disclosed hereafter. In some embodiments, a method as disclosed herein can comprise contacting in vitro or ex vivo the immune cell of a subject with a lipid nanoparticle (LNP). In some embodiments, the LNP is an LNP as described herein in the present disclosure.

In some aspects, provided are methods of expressing a polypeptide of interest in a targeted immune cell of a subject. In some embodiments, the method comprises contacting the immune cell with a lipid nanoparticle (LNP). In some embodiments, the LNP comprises an ionizable cationic lipid. In some embodiments, the LNP comprises a conjugate comprising the following structure of Formula (II): [Lipid]-[optional linker]-[immune cell targeting group]. In some embodiments, the LNP comprises a sterol or other structural lipid. In some embodiments, the LNP comprises a neutral phospholipid. In some embodiments, the LNP comprises a free Polyethylene glycol (PEG) lipid. In some embodiments, the LNP comprises a nucleic acid encoding the polypeptide. In some embodiments, an aspect of the disclosure relates to an LNP or a pharmaceutical composition containing thereof, as disclosed herein, for use in a method of expressing a polypeptide of interest in a targeted immune cell of a subject. Such a method may be for the treatment of a disease or disorder as disclosed hereafter. In some embodiments, a method as disclosed herein can comprise contacting in vitro or ex vivo the immune cell of a subject with a lipid nanoparticle (LNP).

In some aspects, provided are methods of modulating cellular function of a target immune cell of a subject. In some embodiments, the method comprises administering to the subject a lipid nanoparticle (LNP). In some embodiments, the LNP comprises an ionizable cationic lipid. In some embodiments, the LNP comprises a conjugate comprising the following structure of Formula (II): [Lipid]-[optional linker]-[immune cell targeting group]. In some embodiments, the LNP comprises a sterol or other structural lipid. In some embodiments, the LNP comprises a neutral phospholipid. In some embodiments, the LNP comprises a free Polyethylene glycol (PEG) lipid. In some embodiments, the LNP comprises a nucleic acid encoding a polypeptide for modulating the cellular function of the immune cell. In some embodiments, an aspect of the disclosure relates to an LNP or a pharmaceutical composition containing thereof, as disclosed herein, for use in a method of modulating cellular function of a targeted immune cell of a subject. Such a method may be for the treatment of a disease or disorder as disclosed hereafter. In some embodiments, a method as disclosed herein can comprise contacting in vitro or ex vivo the immune cell of a subject with a lipid nanoparticle (LNP).

In some embodiments, the modulation of cell function comprises reprogramming the immune cells to initiate an immune response. In some embodiments, the modulation of cell function comprises modulating antigen specificity of the immune cell.

In some aspects, provided are methods of treating, ameliorating, or preventing a symptom of a disorder or disease in a subject in need thereof. In any of the embodiments described herein concerning a method of treating, ameliorating, and/or preventing a symptom of a disorder or disease by administration of, e.g., a LNP of the invention, it is intended that said disclosures are also interpreted as providing the, e.g., LNP for use in said methods of treating, ameliorating, and/or preventing a symptom of a disorder or disease. In some embodiments, the method comprises administering to the subject a lipid nanoparticle (LNP) for delivering a nucleic acid into an immune cell of the subject. In some embodiments, the LNP comprises an ionizable cationic lipid. In some embodiments, the LNP comprises a conjugate comprising the following structure of Formula (II): [Lipid]-[optional linker]-[immune cell targeting group]. In some embodiments, the LNP comprises a sterol or other structural lipid. In some embodiments, the LNP comprises a neutral phospholipid. In some embodiments, the LNP comprises a free Polyethylene glycol (PEG) lipid. In some embodiments, the LNP comprises the nucleic acid.

In some embodiments, the nucleic acid modulates the immune response of the immune cell, therefore to treat or ameliorate the symptom. In some embodiments, an aspect of the disclosure relates to an LNP or a pharmaceutical composition containing thereof, as disclosed herein, for use in a method of treating, ameliorating, or preventing a symptom of a disorder or disease in a subject in need thereof. A disease or disorder may be as disclosed hereafter. In some embodiments, a method as disclosed herein can comprise contacting in vitro or ex vivo the immune cell of a subject with a lipid nanoparticle (LNP).

In some embodiments, the disorder is an immune disorder, an inflammatory disorder, or cancer. In some embodiments, the nucleic acid encodes an antigen for use in a therapeutic or prophylactic vaccine for treating or preventing an infection by a pathogen. In some embodiments, the Fab antibody comprises a heavy chain fragment that comprises F174C substitution, numbering according to Kabat, and/or a light chain fragment that comprises S176C substitution, numbering according to Kabat

In some embodiments, no more than 1%, 2%, 3%, 4%, 5%, 6%, 7%, 8%, 9%, or 10% of non-immune cells are transfected by the LNP. In some embodiments, no more than 1%, 2%, 3%, 4%, 5%, 6%, 7%, 8%, 9%, or 10% of undesired immune cells that are not meant to be the destination of the delivery are transfected by the LNP. In some embodiments, the half-life of the nucleic acid delivered by the LNP to the immune cell or a polypeptide encoded by the nucleic acid delivered by the LNP is at least 5%, 10%, 15%, 20%, 25%, 30%, 40%, 50%, 60%, 70%, 80%, 90%, 100%, 1.5 times, 2 times, 3 times, 4 times, 5 times, 10 times, or longer than the half-life of nucleic acid delivered by a reference LNP to the immune cell or a polypeptide encoded by the nucleic acid delivered by the reference LNP.

In some embodiments, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or more immune cells that are meant to be the destination of the delivery are transfected by the LNP.

In some embodiments, expression level of the nucleic acid delivered by the LNP is at least 5%, at least 10%, at least 10%, at least 10%, at least 10%, at least 10%, at least 10%, at least 10%, at least 10%, at least 10%, at least 10%, at least 10%, at least 10%, at least 10%, at least 10%, 1.5 time, 2 times, 3 times, 4 times, 5 times, 10 times, 15 times, 20 times or more higher than expression level of nucleic acid in the same immune cells delivered by a reference LNP.

In one aspect, provided are lipid nanoparticles (LNPs) for delivering a nucleic acid into NK cells of the subject. The LNP comprises (a) An ionizable cationic lipid, (b) A conjugate comprising the following structure: [Lipid]-[optional linker]-[immune cell targeting group]; (c) A sterol or other structural lipid; (d) A neutral phospholipid; (e) A free Polyethylene glycol (PEG) lipid, and (f) the nucleic acid. In some embodiments, the immune cell targeting group comprises an antibody that binds CD56.

In one aspect, provided are lipid nanoparticles (LNPs) for delivering a nucleic acid into immune cells of the subject. The LNP comprises (a) An ionizable cationic lipid, (b) A conjugate comprising the following structure: [Lipid]-[optional linker]-[immune cell targeting group]; (c) A sterol or other structural lipid; (d) A neutral phospholipid; (e) A free Polyethylene glycol (PEG) lipid, and (f) the nucleic acid. In some embodiments, the immune cell targeting group comprises an antibody that binds CD7 or CD8, and the free PEG lipid is DMG-PEG or DPG-PEG.

In one aspect, provided are lipid nanoparticles (LNPs) for delivering a nucleic acid into immune cells of the subject. The LNP comprises (a) An ionizable cationic lipid, (b) A conjugate comprising the following structure: [Lipid]-[optional linker]-[immune cell targeting group]; (c) A sterol or other structural lipid; (d) A neutral phospholipid; (e) A free Polyethylene glycol (PEG) lipid, and (f) the nucleic acid. In some embodiments, the immune cell targeting group comprises an antibody, and the antibody is a Fab or an immunoglobulin single variable domain. In some embodiments, the Fab is engineered to knock out the natural interchain disulfide at the C-terminus. In some embodiments, the Fab has a buried interchain disulfide. In some embodiments, the antibody is an immunoglobulin single variable (ISV) domain, and the ISV domain an Nanobody® ISV. In some embodiments, the free PEG lipid comprise a PEG having a molecular weight of at least 2000 daltons. In some embodiments, the PEG has a molecular weight of about 3000 to 5000 daltons. In some embodiments, the Fab is an anti-CD3 antibody, and the free PEG lipid in the LNP comprises a PEG having a molecular weight of about 2000 daltons. In some embodiments, the Fab is an anti-CD4 antibody, and the free PEG lipid in the LNP comprises a PEG having a molecular weight of about 3000 to 3500 daltons.

In some embodiments, the free PEG-lipid comprises a diacylphosphatidylethanolamine, a dialkylphosphatidylethanolamine, a diacylglycerol, a ceramide, a dialkylglycerol, or a dialkylacetamide. In some embodiments, the alkyl chain is myristic acid, palmitic acid, oleic acid, linoleic acid, or stearic acid. In some embodiments, the free PEG-lipid is DMG-PEG. In some embodiments, free PEG-lipid is DPG-PEG.

In one aspect, provided are lipid nanoparticles (LNPs) for delivering a nucleic acid into immune cells of the subject. The LNP comprises (a) An ionizable cationic lipid, (b) A conjugate comprising the following structure: [Lipid]-[optional linker]-[immune cell targeting group]; (c) A sterol or other structural lipid; (d) A neutral phospholipid; (e) A free Polyethylene glycol (PEG) lipid, and (f) the nucleic acid. In some embodiments, the immune cell targeting group comprises an antibody that binds CD3, and an antibody that binds CD11a or CD18.

In one aspect, provided are lipid nanoparticles (LNPs) for delivering a nucleic acid into immune cells of the subject. The LNP comprises (a) An ionizable cationic lipid, (b) A conjugate comprising the following structure: [Lipid]-[optional linker]-[immune cell targeting group]; (c) A sterol or other structural lipid; (d) A neutral phospholipid; (e) A free Polyethylene glycol (PEG) lipid, and (f) the nucleic acid. In some embodiments, the immune cell targeting group comprises an antibody that binds CD7, and an antibody that binds CD8.

In one aspect, provided are lipid nanoparticles (LNPs) for delivering a nucleic acid into two different types of immune cells of the subject. The LNP comprises (a) An ionizable cationic lipid, (b) A conjugate comprising the following structure: [Lipid]-[optional linker]-[immune cell targeting group]; (c) A sterol or other structural lipid; (d) A neutral phospholipid; (e) A free Polyethylene glycol (PEG) lipid, and (f) the nucleic acid.

In some embodiments, the immune cell targeting group comprise a bispecific targeting moiety. In some embodiments, the bispecific targeting moiety binds to the two different types of immune cells. In some embodiments, the two different types of immune cells are CD4+ T cells and CD8+ T cell. In some embodiments, the bispecific targeting moiety is a bispecific antibody. In some embodiments, the bispecific antibody is a Fab-ScFv.

In one aspect, provided are lipid nanoparticles (LNPs) for delivering a nucleic acid into both CD4+ and CD8+ T cells of a subject. The LNP comprises (a) An ionizable cationic lipid, (b) A conjugate comprising the following structure: [Lipid]-[optional linker]-[immune cell targeting group]; (c) A sterol or other structural lipid; (d) A neutral phospholipid; (e) A free Polyethylene glycol (PEG) lipid, and (f) the nucleic acid. In some embodiments, the immune cell targeting group comprise a single antibody that binds to CD3 or CD7.

Further provided is a lipid nanoparticle (LNP) for delivering a nucleic acid into an immune cell of a subject, wherein the LNP comprises: (a) an ionizable cationic lipid, (b) a conjugate comprising the following structure: [Lipid]-[optional linker]-[immune cell targeting group]; (c) a sterol or other structural lipid; (d) a neutral phospholipid; (e) a free Polyethylene glycol (PEG) lipid, and (f) the nucleic acid, wherein the immune cell targeting group comprises a Fab lacking the native interchain disulfide bond. In some embodiments, the Fab is engineered to replace one or both cysteines on the native constant light chain and the native constant heavy chain that form the native interchain disulfide with a non-cysteine amino acid, therefor to remove the native interchain disulfide bond in the Fab.

Also provided is an immunoglobulin single variable domain (ISVD) that binds to human CD8. In some embodiments, the ISVD comprises three complementarity determining domains CDR1, CDR2, and CDR3, wherein
(a) the CDR1 comprises GSTFSDYG (SEQ ID NO: 100),
(b) the CDR2 comprises IDWNGEHT (SEQ ID NO: 101), and
(c) the CDR3 comprises AADALPYTVRKYNY (SEQ ID NO: 102).

In some embodiments, the ISVD is humanized.

In some embodiments, the ISVD comprises, consists of, or consists essentially of SEQ ID NO: 77.

Also provided is a polypeptide comprising GSTFSDYG (SEQ ID NO: 100), IDWNGEHT (SEQ ID NO: 101), and AADALPYTVRKYNY (SEQ ID NO: 102).

In some embodiments, the polypeptide comprises the ISVD as described herein.

In some embodiments, the polypeptide further comprises a second binding moiety, wherein the second binding moiety binds to CD8 or another different target. In some embodiments, the second binding moiety is also an ISVD.

In some embodiments, the polypeptide further comprises a detectable marker, or a therapeutic agent.

Also provided is a composition comprising the ISVD or the polypeptide as described herein.

Further provided is a pharmaceutical composition comprising the ISVD or the polypeptide as described herein, and a pharmaceutically acceptable carrier.

Further provided is a method of treating a disease or disorder related to CD8 in a subject, comprising administering the pharmaceutical composition as described herein to the subject.

In some embodiments, the disease is cancer. In some embodiments, the disorder is an immune disorder, an inflammatory disorder, or cancer.

In some embodiments, the nucleic acid encodes an antigen for use in a therapeutic or prophylactic vaccine for treating or preventing an infection by a pathogen. In some embodiments, the ionizable cationic lipid is an ionizable cationic lipid as disclosed herein, such as those in Table 1.

In some embodiments, the immune cell targeting group comprises an antibody that binds a T cell antigen. In some embodiments, the T cell antigen is CD3, CD8, or both CD3 and CD8.60. In some embodiments, the immune cell targeting group comprises an antibody that binds a Natural Killer (NK) cell antigen. In some embodiments, the NK cell antigen is CD56. In some embodiments, the antibody is a human or humanized antibody.

In some embodiments, the immune cell targeting group is covalently coupled to a lipid in the lipid blend via a polyethylene glycol (PEG) containing linker. In some embodiments, the lipid covalently coupled to the immune cell targeting group via a PEG containing linker is distearoylglycerol (DSG), distearoyl-phosphatidylethanolamine (DSPE), dimyrstoyl-phosphatidylethanolamine (DMPE), distearoyl-glycero-phosphoglycerol (DSPG), dimyristoyl-glycerol (DMG), dipalmitoyl-phosphatidylethanolamine (DPPE), dipalmitoyl-glycerol (DPG), or ceramide. In some embodiments, the PEG is PEG 2000. In some embodiments, the PEG is PEG 3400.

In some embodiments, the lipid-immune cell targeting group conjugate is present in the lipid blend in a range of 0.002-0.2 mole percent. In some embodiments, the ionizable cationic lipid is present in the lipid blend in a range of 40-60 mole percent.

In some embodiments, the sterol is cholesterol. In some embodiments, the sterol is present in the lipid blend in a range of 30-50 mole percent. In some embodiments, the neutral phospholipid is selected from the group consisting of phosphatidylcholine, phosphatidylethanolamine, distearoyl-sn-glycero-3-phosphoethanolamine (DSPE), 1,2-distearoyl-sn-glycero-3-phosphocholine (DSPC), hydrogenated soy phosphatidylcholine (HSPC), 1,2-dioleoyl-sn-glycero-3-phosphoethanolamine (DOPE), 1,2-dioleoyl-sn-glycero-3-phosphocholine (DOPC), sphingomyelin (SM).

In some embodiments, the neutral phospholipid is present in the lipid blend in a range of 1-15 mole percent, such as about 5 to 15 mole percent, or about 5 to 10 mole percent.

In some embodiments, the free PEG-lipid is selected from the group consisting of PEG-modified phosphatidylethanolamines, PEG-modified phosphatidic acids, PEG-modified ceramides, PEG-modified dialkylamines, PEG-modified diacylglycerols, and PEG-modified dialkylglycerols. For example, a PEG lipid may be PEG-dioleoylgylcerol (PEG-DOG), PEG-dimyristoyl-glycerol (PEG-DMG), PEG-dipalmitoyl-glycerol (PEG-DPG), PEG-dilinoleoyl-glycero-phosphatidyl ethanolamine (PEG-DLPE), PEG-dimyrstoyl-phosphatidylethanolamine (PEG-DMPE), PEG-dipalmitoyl-phosphatidylethanolamine (PEG-DPPE), PEG-distearoylglycerol (PEG-DSG), PEG-diacylglycerol (PEG-DAG, e.g., PEG-DMG, PEG-DPG, and PEG-DSG), PEG-ceramide, PEG-distearoyl-glycero-phosphoglycerol (PEG-DSPG), PEG-dioleoyl-glycero-phosphoethanolamine (PEG-DOPE), 2-[(polyethylene glycol)-2000]-N,N-ditetradecylacetamide, or a PEG-distearoyl-phosphatidylethanolamine (PEG-DSPE) lipid.

In some embodiments, the free PEG-lipid comprises a diacylphosphatidylethanolamines comprising Dipalmitoyl (C16) chain or Distearoyl (C18) chain. In some embodiments, the free PEG-lipid is present in the lipid blend in about 0.1-4 mole percent, such as 0.5 to 2.5 mole percent, or about 1 to 2 mole percent. In some embodiments, the free PEG-lipid is present in the lipid blend in about 1.5 mole percent. In some embodiments, the free PEG-lipid comprises the same or a different lipid as the lipid in the lipid-immune cell targeting group conjugate.

In some embodiments, the LNP has a mean diameter in the range of 50-200 nm. In some embodiments, the LNP has a mean diameter of about 100 nm. In some embodiments, the LNP has a polydispersity index in a range from 0.05 to 1. In some embodiments, the LNP has a zeta potential of from about +5 mV to about +50 mV at pH5, such as about +10 mV to about +30 mV at pH 5. In some embodiments, the LNP has a zeta potential of from about −10 mV to about +10 mV at pH 7.4.

In some embodiments, the nucleic acid is DNA or RNA. In some embodiments, the RNA is an mRNA, tRNA, siRNA, gRNA (guide RNA), circRNA (circular RNA), ribozymes, decoy RNA, or microRNA. In some embodiments, the mRNA encodes a receptor, a growth factor, a hormone, a cytokine, an antibody, an antigen, an enzyme, or a vaccine. In some embodiments, the mRNA encodes a polypeptide capable of regulating immune response in the immune cell. In some embodiments, the mRNA encodes a polypeptide capable of reprogramming the immune cell. In some embodiments, the mRNA encodes a synthetic T cell receptor (synTCR) or a Chimeric Antigen Receptor (CAR).

In some embodiments, the immune cell targeting group comprises an antibody, and the antibody is a Fab or an immunoglobulin single variable domain. In some embodiments, the immune cell targeting group comprises an antibody fragment selected from the group consisting of a Fab, F(ab′)2, Fab′-SH, Fv, and scFv fragment. In some embodiments, the immune cell targeting group comprises a Fab that comprises one or more interchain disulfide bonds. In some embodiments, the Fab comprises a heavy chain fragment that comprises F174C and C233S substitutions, and a light chain fragment that comprises S176C and C214S substitutions, numbering according to Kabat. In some embodiments, the immune cell targeting group comprises a Fab that comprises a cysteine at the C-terminus of the heavy or light chain fragment.

In some embodiments, the Fab further comprises one or more amino acids between the heavy chain fragment of the Fab and the C-terminal cysteine. In some embodiments, the Fab comprises a heavy chain variable domain linked to an antibody CH1 domain and a light chain variable domain linked to an antibody light chain constant domain. In some embodiments, the CH1 domain and the light chain constant domain are linked by one or more interchain disulfide bonds. In some embodiments, the immune cell targeting group further comprises a single chain variable fragment (scFv) linked to the C-terminus of the light chain constant domain by an amino acid linker.

In some embodiments, the immune cell targeting group comprises an immunoglobulin single variable domain. In some embodiments, the immunoglobulin single variable domain comprises a cysteine at the C-terminus. In some embodiments, the immunoglobulin single variable domain comprises a VHH domain and further comprises a spacer comprising one or more amino acids between the VHH domain and the C-terminal cysteine. In some embodiments, the immune cell targeting group comprises two or more VHH domains. In some embodiments, the two or more VHH domains are linked by an amino acid linker. In some embodiments, the immune cell targeting group comprises a first VHH domain linked to an antibody CH1 domain and a second VHH domain linked to an antibody light chain constant domain. In some embodiments, the antibody CH1 domain and the antibody light chain constant domain are linked by one or more disulfide bonds. In some embodiments, the immune cell targeting group comprises a VHH domain linked to an antibody CH1 domain. In some embodiments, the antibody CH1 domain is linked to an antibody light chain constant domain by one or more disulfide bonds. In some embodiments, the CH1 domain comprises F174C and C233S substitutions, and the light chain constant domain comprises S176C and C214S substitutions, numbering according to Kabat.

In some embodiments, the immune cell targeting group comprises a Fab that comprises:
(a) a heavy chain fragment comprising the amino acid sequence of SEQ ID NO: 1 and a light chain fragment comprising the amino acid sequence of SEQ ID NO:2 or 3;
(b) a heavy chain fragment comprising the amino acid sequence of SEQ ID NO: 6 and a light chain fragment comprising the amino acid sequence of SEQ ID NO: 7.

In some embodiments, no more than 5% of non-immune cells are transfected by the LNP. In some embodiments, the half-life of the nucleic acid delivered by the LNP or a polypeptide encoded by the nucleic acid delivered by the LNP is at least 10% longer than the half-life of a nucleic acid delivered by a reference LNP or a polypeptide encoded by the nucleic acid delivered by a reference LNP. In some embodiments, at least 10% of immune cells are transfected by the LNP. In some embodiments, expression level of the nucleic acid delivered by the LNP is at least 10% higher than expression level of a nucleic acid delivered by a reference LNP.

In some aspects, provided are lipid nanoparticles (LNPs) for delivering a nucleic acid into an immune cell of the subject. In some embodiments, the LNP comprises an ionizable cationic lipid. In some embodiments, the LNP comprises a conjugate comprising the following structure: [Lipid]-[optional linker]-[immune cell targeting group]. In some embodiments, the LNP comprises a sterol or other structural lipid. In some embodiments, the LNP comprises a neutral phospholipid. In some embodiments, the LNP comprises free Polyethylene glycol (PEG) lipid. In some embodiments, the LNP comprises the nucleic acid. In some embodiments, the immune cell is an NK cell. In some embodiments, the immune cell targeting group comprises an antibody that binds CD56.

In some aspect, provided herein are lipid nanoparticles (LNPs) for delivering a nucleic acid into an immune cell of the subject. In some embodiments, the LNP comprises an ionizable cationic lipid. In some embodiments, the LNP comprises a conjugate comprising the following structure: [Lipid]-[optional linker]-[immune cell targeting group]. In some embodiments, the LNP comprises a sterol or other structural lipid. In some embodiments, the LNP comprises a neutral phospholipid. In some embodiments, the LNP comprises a free Polyethylene glycol (PEG) lipid. In some embodiments, the LNP comprises the nucleic acid. In some embodiments, the immune cell targeting group comprises an antibody that binds CD7 or CD8. In some embodiments, the free PEG lipid is DMG-PEG or DPG-PEG.

In some aspects, provided are lipid nanoparticles (LNPs) for delivering a nucleic acid into an immune cell of the subject. In some embodiments, the LNP comprises an ionizable cationic lipid. In some embodiments, the LNP comprises a conjugate comprising the following structure: [Lipid]-[optional linker]-[immune cell targeting group]. In some embodiments, the LNP comprises a sterol or other structural lipid. In some embodiments, the LNP comprises neutral phospholipid. In some embodiments, the LNP comprises a free Polyethylene glycol (PEG) lipid. In some embodiments, the LNP comprises the nucleic acid. In some embodiments, the immune cell targeting group comprises an antibody. In some embodiments, the antibody is a Fab or an immunoglobulin single variable domain.

In some embodiments, the Fab is engineered to knock out the natural interchain disulfide at the C-terminus. In some embodiments, the Fab comprises a heavy chain fragment that comprises C233S substitutions, and a light chain fragment that comprises C214S substitutions. In some embodiments, the Fab comprises a non-natural interchain disulfide. In some embodiments, the Fab comprises F174C substitution in the heavy chain fragment, and S176C substitution in the light chain fragment. In some embodiments, the antibody is an immunoglobulin single variable (ISV) domain, and the ISV domain is an Nanobody® ISV. In some embodiments, the free PEG lipid comprises a PEG having a molecular weight of at least 2000 daltons. In some embodiments, the PEG has a molecular weight of about 3000 to 5000 daltons. In some embodiments, the antibody is a Fab. In some embodiments, the Fab binds CD3, and the free PEG lipid in the LNP comprises a PEG having a molecular weight of about 2000 daltons. In some embodiments, the Fab is an anti-CD4 antibody, and the free PEG lipid in the LNP comprises a PEG having a molecular weight of about 3000 to 3500 daltons.

In some embodiments, the immune cell targeting group comprises two or more VHH domains. In some embodiments, the two or more VHH domains are linked by an amino acid linker. In some embodiments, the immune cell targeting group comprises a first VHH domain linked to an antibody CH1 domain and a second VHH domain linked to an antibody light chain constant domain.

In some aspects, provided are lipid nanoparticles (LNPs) for delivering a nucleic acid into an immune cell of the subject. In some embodiments, the LNP comprises an ionizable cationic lipid. In some embodiments, the LNP comprises a conjugate comprising the following structure: [Lipid]-[optional linker]-[immune cell targeting group]. In some embodiments, the LNP comprises a sterol or other structural lipid. In some embodiments, the LNP comprises a neutral phospholipid. In some embodiments, the LNP comprises a free Polyethylene glycol (PEG) lipid. In some embodiments, the LNP comprises the nucleic acid.

In some embodiments, the LNP binds CD3, and also binds CD11a or CD18. In some embodiments, the LNP comprises two conjugates. In some embodiments, the first conjugate comprises an antibody that binds CD3. In some embodiments, the second conjugate comprises an antibody that binds CD11a or CD18. In some embodiments, the LNP comprises one conjugate. In some embodiments, the conjugate comprises a bispecific antibody that binds both CD3 and CD11a. In some embodiments, the conjugate comprises a bispecific antibody that binds both CD3 and CD18. In some embodiments, the bispecific antibody is an immunoglobulin single variable domain or Fab-ScFv. In some aspects, provided are lipid nanoparticles (LNPs) for delivering a nucleic acid into an immune cell of the subject. In some embodiments, the LNP comprises an ionizable cationic lipid. In some embodiments, the LNP comprises a conjugate comprising the following structure: [Lipid]-[optional linker]-[immune cell targeting group]. In some embodiments, the LNP comprises a sterol or other structural lipid. In some embodiments, the LNP comprises a neutral phospholipid. In some embodiments, the LNP comprises a free Polyethylene glycol (PEG) lipid. In some embodiments, the LNP comprises the nucleic acid. In some embodiments, the LNP binds CD7 and CD8 of the immune cell.

In some embodiments, the LNP comprises two conjugates. In some embodiments, the first conjugate comprises an antibody that binds CD7, and a second conjugate that binds CD8. In some embodiments, the LNP comprises one conjugate. In some embodiments, the conjugate comprises a bispecific antibody that binds CD7 and CD8. In some embodiments, the bispecific antibody is an immunoglobulin single variable domain or Fab-ScFv.

In some aspects, provided are lipid nanoparticles (LNPs) for delivering a nucleic acid into two different types of immune cells of the subject. In some embodiments, the LNP comprises: an ionizable cationic lipid. In some embodiments, the LNP comprises a conjugate comprising the following structure: [Lipid]-[optional linker]-[immune cell targeting group]. In some embodiments, the LNP comprises sterol or other structural lipid. In some embodiments, the LNP comprises neutral phospholipid. In some embodiments, the LNP comprises a free Polyethylene glycol (PEG) lipid. In some embodiments, the LNP comprises the nucleic acid. In some embodiments, the LNP binds to a first antigen on the surface of the first type of immune cell, and also binds to a second antigen on the surface of the second type of immune cell. In some embodiments, the two different types of immune cells are CD4+ T cells and CD8+ T cell. In some embodiments, the LNP comprises two conjugates. In some embodiments, the first conjugate comprises a first antibody that binds to the first antigen of the first type of immune cell, and the second conjugate comprises a second antibody that binds to the second antigen of the second type of immune cell. In some embodiments, the LNP comprises one conjugate. In some embodiments, the conjugate comprises a bispecific antibody. In some embodiments, the bispecific antibody binds to both the first antigen on the first type of immune cell, and the second antigen on the second type of immune cells. In some embodiments, the bispecific antibody is an immunoglobulin single variable domain or a Fab-ScFv.

In some aspects, provided are lipid nanoparticles (LNPs) for delivering a nucleic acid into both CD4+ and CD8+ T cells of a subject. In some embodiments, the LNP comprises an ionizable cationic lipid. In some embodiments, the LNP comprises a conjugate comprising the following structure: [Lipid]-[optional linker]-[immune cell targeting group]. In some embodiments, the LNP comprises a sterol or other structural lipid. In some embodiments, the LNP comprises a neutral phospholipid. In some embodiments, the LNP comprises a free Polyethylene glycol (PEG) lipid. In some embodiments, the LNP comprises the nucleic acid. In some embodiments, the immune cell targeting group comprises a single antibody that binds to CD3 or CD7.

In some aspects, provided are lipid nanoparticles (LNPs) for delivering a nucleic acid into both T cells and NK cells of a subject. In some embodiments, the LNP comprises an ionizable cationic lipid. In some embodiments, the LNP comprises a conjugate comprising the following structure: [Lipid]-[optional linker]-[immune cell targeting group]. In some embodiments, the LNP comprises sterol or other structural lipid. In some embodiments, the LNP comprises a neutral phospholipid. In some embodiments, the LNP comprises a free Polyethylene glycol (PEG) lipid. In some embodiments, the LNP comprises the nucleic acid. In some embodiments, the immune cell targeting group binds to CD7, CD8, or both CD7 and CD8.

In some aspects, provided are lipid nanoparticles (LNPs) for delivering a nucleic acid into both T cells and NK cells of a subject. In some embodiments, the LNP comprises an ionizable cationic lipid. In some embodiments, the LNP comprises a conjugate comprising the following structure: [Lipid]-[optional linker]-[immune cell targeting group]. In some embodiments, the LNP comprises a sterol or another structural lipid. In some embodiments, the LNP comprises a neutral phospholipid. In some embodiments, the LNP comprises a free Polyethylene glycol (PEG) lipid. In some embodiments, the LNP comprises the nucleic acid. In some embodiments, the immune cell targeting group binds to (i) both CD3 and CD56; (ii) both CD8 and CD56; or (iii) both CD7 and CD56.

In some embodiments, the immune cell targeting group is covalently coupled to a lipid in the lipid blend via a polyethylene glycol (PEG) containing linker. In some embodiments, the lipid covalently coupled to the immune cell targeting group via a PEG containing linker is distearoylglycerol (DSG), distearoyl-phosphatidylethanolamine (DSPE), dimyrstoyl-phosphatidylethanolamine (DMPE), distearoyl-glycero-phosphoglycerol (DSPG), dimyristoyl-glycerol (DMG), dipalmitoyl-phosphatidylethanolamine (DPPE), dipalmitoyl-glycerol (DPG), dialkylacetamide, or ceramide. In some embodiments, the lipid-immune cell targeting group conjugate is present in the lipid blend in a range of 0.002-0.2 mole percent. In some embodiments, the lipid blend further comprises one or more of a structural lipid (e.g., a sterol), a neutral phospholipid, and a free PEG-lipid.

In some embodiments, the ionizable cationic lipid is present in the lipid blend in a range of 40-60 mole percent.

In some embodiments, the sterol is present in the lipid blend in a range of 30-50 mole percent. In some embodiments, the sterol is cholesterol.

In some embodiments, the neutral phospholipid is selected from the group consisting of phosphatidylcholine, phosphatidylethanolamine, distearoyl-sn-glycero-3-phosphoethanolamine (DSPE), hydrogenated soy phosphatidylcholine (HSPC), 1,2-distearoyl-sn-glycero-3-phosphocholine (DSPC), 1,2-dioleoyl-sn-glycero-3-phosphoethanolamine (DOPE), 1,2-dioleoyl-sn-glycero-3-phosphocholine (DOPC). In some embodiments, the neutral phospholipid is present in the lipid blend in a range of 1-15 mole percent, such as about 5-15 mole percent.

In some embodiments, the free PEG-lipid is selected from the group consisting of PEG-distearoyl-phosphatidylethanolamine (PEG-DSPE) or PEG-dimyrstoyl-phosphatidylethanolamine (PEG-DMPE), N-(Methylpolyoxyethylene oxycarbonyl)-1,2-dipalmitoyl-sn-glycero-3-phosphoethanolamine (DPPE-PEG) 1,2-Dimyristoyl-rac-glycero-3-methylpolyoxyethylene (PEG-DMG), 1,2-Dipalmitoyl-rac-glycero-3-methylpolyoxyethylene (PEG-DPG), 1,2-Dioleoyl-rac-glycerol, methoxypolyethylene Glycol (DOG-PEG) 1,2-Distearoyl-rac-glycero-3-methylpolyoxyethylene (PEG-DSG), N-palmitoyl-sphingosine-1-{succinyl[methoxy(polyethylene glycol)] (PEG-ceramide), and DSPE-PEG-cysteine, or a derivative thereof. In some embodiments, the free PEG-lipid comprises a diacylphosphatidylethanolamines comprising Dipalmitoyl (C16) chain or Distearoyl (C18) chain. In some embodiments, the free PEG-lipid is present in the lipid blend in a range of 0.1 to 4 mole percent, such as about 0.5-2.5 mole percent. In some embodiment, the free PEG-lipid is about 1.5 mole percent. In some embodiments, the free PEG-lipid comprises the same or a different lipid as the lipid in the lipid-immune cell targeting group conjugate.

In some embodiments, the LNP has a mean diameter in the range of 50-200 nm. In some embodiments, the LNP has a mean diameter of about 100 nm. In some embodiments, the LNP has a polydispersity index in a range from 0.05 to 1. In some embodiments, the LNP has a zeta potential of from about −5 mV to 50 mV at pH 5, such as about +10 mV to about +30 mV at pH 5. In some embodiments, the LNP has a zeta potential of from about −10 mV to about +10 mV at pH 7.4.

In some embodiments, the nucleic acid is DNA or RNA. In some embodiments, the RNA is an mRNA. In some embodiments, the mRNA encodes a receptor, a growth factor, a hormone, a cytokine, an antibody, an antigen, an enzyme, or a vaccine. In some embodiments, the mRNA encodes a polypeptide capable of regulating immune response in the immune cell. In some embodiments, the mRNA encodes a polypeptide capable of reprogramming the immune cell. In some embodiments, the mRNA encodes a synthetic T cell receptor (synTCR) or a Chimeric Antigen Receptor (CAR).

In some aspects, provided are lipid nanoparticles (LNPs) for delivering a nucleic acid into an immune cell of a subject. In some embodiments, the LNP comprises an ionizable cationic lipid. In some embodiments, the LNP comprises a conjugate comprising the following structure: [Lipid]-[optional linker]-[immune cell targeting group]. In some embodiments, the LNP comprises a sterol or other structural lipid. In some embodiments, the LNP comprises a neutral phospholipid. In some embodiments, the LNP comprises a free Polyethylene glycol (PEG) lipid. In some embodiments, the LNP comprises the nucleic acid.

In some embodiments, the immune cell targeting group comprises a Fab lacking the native interchain disulfide bond. In some embodiments, the Fab is engineered to replace one or both cysteines on the native constant light chain and the native constant heavy chain that form the native interchain disulfide with a non-cysteine amino acid, therefor to remove the native interchain disulfide bond in the Fab.

In some aspect, provided are methods of targeting the delivery of a nucleic acid to an immune cell of a subject. In some embodiments, the method comprises contacting the immune cell with a lipid nanoparticle (LNP) provided herein. In some embodiments, the method is for targeting NK cells. In some embodiments, the immune cell targeting group binds to CD56. In some embodiments, the method is for targeting both T cells and NK cells simultaneously. In some embodiments, the immune cell targeting group binds to CD7, CD8, or both CD7 and CD8. In some embodiments, the method is for targeting both CD4+ and CD8+ T cells simultaneously. In some embodiments, the immune cell targeting group comprises a polypeptide that binds to CD3 or CD7.

In some aspect, provided are methods of expressing a polypeptide of interest in a targeted immune cell of a subject. In some embodiments, the method comprises contacting the immune cell with a lipid nanoparticle (LNP) provided herein.

In some aspect, provided are methods of modulating cellular function of a target immune cell of a subject. In some embodiments, the methods comprise administering to the subject a lipid nanoparticle (LNP) provided herein.

In some aspect, provided are methods of treating, ameliorating, or preventing a symptom of a disorder or disease in a subject in need thereof. In some embodiments, the methods comprise administering to the subject a lipid nanoparticle (LNP) provided herein.

In some aspects, provided are immunoglobulin single variable domains (ISVDs) that bind to human CD8. In some embodiments, the ISVD comprises three complementarity determining domains CDR1, CDR2, and CDR3. In some embodiments, the CDR1 comprises GSTFSDYG (SEQ ID NO: 100). In some embodiments, the CDR2 comprises IDWNGEHT (SEQ ID NO: 101). In some embodiments, the CDR3 comprises AADALPYTVRKYNY (SEQ ID NO: 102). In some embodiments, the ISVD is humanized. In some embodiments, the ISVD comprises SEQ ID NO: 77.

In some aspects, provided are polypeptides comprising GSTFSDYG (SEQ ID NO: 100), IDWNGEHT (SEQ ID NO: 101), and AADALPYTVRKYNY (SEQ ID NO: 102). In another aspect, provided are polypeptides comprising the ISVD provided herein. In some embodiments, the polypeptide comprises a second binding moiety. In some embodiments, the second binding moiety binds to CD8 or another different target. In some embodiments, the second binding moiety is also an ISVD. In some embodiments, the polypeptide comprises a detectable marker. In some embodiments, the polypeptide comprises a therapeutic agent.

In some aspects, provided are compositions comprising the ISVD provided herein or the polypeptide provided herein.

In some aspects, provided are pharmaceutical compositions comprising the ISVD provided herein or the polypeptide provided herein, and a pharmaceutically acceptable carrier.

In some aspects, provided are methods of treating a disease or disorder related to CD8 in a subject. In some embodiments, the method comprises administering a pharmaceutical composition described herein to the subject. In some embodiments, the disease or disorder is cancer.

Various aspects and embodiments of the invention are described in further detail below.

### BRIEF DESCRIPTION OF THE DRAWINGS

FIG. 1 depicts proton NMR spectrum of intermediate 13-11.

FIG. 2A depicts proton NMR spectrum of intermediate 13-11a; FIG. 2B depicts proton NMR spectrum of intermediate 13-11b; and FIG. 2C depicts LC-ELSD of intermediate 13-11b.

FIG. 3A depicts proton NMR spectrum of intermediate 13-10; FIG. 3B depicts LC-CAD chromatogram of intermediate 13-10.

FIG. 4A-1 depicts proton NMR spectrum for Lipid 1; FIG. 4A-2 depicts the LC-CAD chromatogram of Lipid 1.

FIG. 4B-1 depicts proton NMR spectrum of Lipid 3; FIG. 4B-2 depicts the LC-CAD chromatogram of Lipid 3.

FIG. 4C-1 depicts proton NMR spectrum of Lipid 4; FIG. 4C-2 depicts the LC-CAD chromatogram L of Lipid 4.

FIG. 4D-1 depicts proton NMR spectrum of Lipid 5A; FIG. 4D-2 depicts the LC-CAD chromatogram of Lipid 5A.

FIG. 4E-1 depicts proton NMR spectrum of Lipid 6; FIG. 4E-2 depicts the LC-CAD chromatogram of Lipid 6.

FIG. 4F-1 depicts proton NMR spectrum of Lipid 7; FIG. 4F-2 depicts the LC-CAD chromatogram of Lipid 7.

FIG. 4G-1 depicts proton NMR spectrum of Lipid 2; FIG. 4G-2 depicts the LC-CAD chromatogram of Lipid 2;

FIG. 4H-1 depicts proton NMR spectrum of Lipid 8; FIG. 4H-2 depicts the LC-CAD chromatogram of Lipid 8.

FIG. 4I-1 depicts proton NMR spectrum of Lipid 9; FIG. 4I-2 depicts the LC-CAD chromatogram of Lipid 9.

FIG. 4J-1 depicts proton NMR spectrum of Lipid 10A; FIG. 4J-2 depicts the LC-CAD chromatogram of Lipid 10A.

FIG. 4K-1 depicts proton NMR spectrum of Lipid 11A; FIG. 4K-2 depicts the LC-CAD chromatogram of Lipid 11A.

FIG. 4L-1 depicts proton NMR spectrum of Lipid 12; FIG. 4L-2 depicts the LC-CAD chromatogram of Lipid 12.

FIG. 4M-1 depicts proton NMR spectrum of Lipid 13; FIG. 4M-2 depicts the LC-CAD chromatogram of Lipid 13.

FIG. 4N-1 depicts proton NMR spectrum of Lipid 15; FIG. 4N-2 depicts the LC-CAD chromatogram of Lipid 15.

FIG. 4O-1 depicts proton NMR spectrum of Lipid 16; FIG. 4O-2 depicts the LC-CAD of Lipid 16.

FIG. 4P-1 depicts proton NMR spectrum of Lipid 19; FIG. 4P-2 depicts the LC-ELSD chromatogram of Lipid 19.

FIG. 4Q-1 depicts proton NMR spectrum of Lipid 20; FIG. 4Q-2 depicts the LC-ELSD chromatogram of Lipid 20.

FIG. 4R-1 depicts proton NMR spectrum of Lipid 31; FIG. 4R-2 depicts the LC-CAD chromatogram of Lipid 31.

FIG. 4S-1 depicts proton NMR spectrum of Lipid 32; FIG. 4S-2 depicts the LC-CAD chromatogram of Lipid 32.

FIG. 4T-1 depicts proton NMR spectrum of Lipid 33; FIG. 4T-2 depicts the LC-CAD chromatogram of Lipid 33.

FIG. 4U-1 depicts proton NMR spectrum of Lipid 34; FIG. 4U-2 depicts the LC-CAD chromatogram of Lipid 34.

FIG. 4V-1 depicts proton NMR spectrum of Lipid 14A; FIG. 4V-2 depicts the LC-CAD chromatogram of Lipid 14A.

FIG. 4W-1 depicts proton NMR spectrum of Lipid 17A; FIG. 4W-1 depicts the LC-CAD chromatogram of Lipid 17A.

FIG. 4X-1 depicts proton NMR spectrum of Lipid 18A; FIG. 4X-2 depicts the LC-CAD chromatogram of Lipid 18A.

FIG. 4Y-1 depicts proton NMR spectrum of Lipid 21A; FIG. 4Y-2 depicts the LC-CAD chromatogram of Lipid 21A.

FIG. 4Z-1 depicts proton NMR spectrum of Lipid 22; FIG. 4Z-2 depicts the LC-CAD chromatogram of Lipid 22.

FIG. 4AA-1 depicts proton NMR spectrum of Lipid 23A; FIG. 4AA-2 depicts the LC-CAD chromatogram of Lipid 23A.

FIG. 4AC-1 depicts proton NMR spectrum of Lipid 25A; FIG. 4AC-2 depicts the LC-CAD chromatogram of Lipid 25A.

FIG. 4AE-1 depicts proton NMR spectrum of Lipid 27; FIG. 4AE-2 depicts the LC-CAD chromatogram of Lipid 27.

FIG. 4AF-1 depicts proton NMR spectrum of Lipid 28; FIG. 4AF-2 depicts the LC-CAD chromatogram of Lipid 28.

FIG. 4AG-1 depicts proton NMR spectrum of Lipid 29; FIG. 4AG-2 depicts the LC-CAD chromatogram of Lipid 29.

FIG. 4AH-1 depicts proton NMR spectrum of Lipid 37A; FIG. 4AH-2 depicts the LC-CAD chromatogram of Lipid 37A.

FIG. 4AI-1 depicts proton NMR spectrum of Lipid 19A; FIG. 4AI-2 depicts the LC-CAD chromatogram of Lipid 19A.

FIG. 4AJ-1 depicts proton NMR spectrum of Lipid 20A; FIG. 4AJ-2 depicts the LC-CAD chromatogram of Lipid 20A.

FIG. 5A depicts diameter (DLS, nm) of LNPs based on Lipid 1 to Lipid 8 in pH 7.4 HBS, pH 6.5 MBS, Post antibody (αCD3, hSP34) insertion and post freeze-thaw (−80° C.).

FIG. 5B depicts polydispersity (DLS) of LNPs based on Lipid 1 to Lipid 8 in pH 7.4 HBS, pH 6.5 MBS, Post antibody (αCD3, hSP34) insertion and post freeze-thaw (−80° C.).

FIG. 5C depicts charge (Zeta potential, DLS) of LNPs based on Lipid 1 to Lipid 8 in pH 5.5 MBS, pH 7.4 HBS.

FIG. 5D depicts % RNA recovery and dye accessible RNA in LNPs based on Lipid 1 to Lipid 8.

FIG. 6A depicts diameter (DLS, nm) of LNPs based on Lipids 9, 10, 11, and 15 in pH 7.4 HBS, pH 6.5 MBS, Post antibody (αCD3, hSP34) insertion and post freeze-thaw (−80° C.).

FIG. 6B depicts polydispersity (DLS) of LNPs based on Lipids 9, 10, 11, and 15 in pH 7.4 HBS, pH 6.5 MBS, Post antibody (αCD3, hSP34) insertion and post freeze-thaw (−80° C.).

FIG. 6C depicts charge (Zeta potential, DLS) of LNPs based on Lipids 9, 10, 11, and 15 in pH 5.5 MBS, pH7.4 HBS.

FIG. 6D depicts % RNA recovery and dye accessible RNA in LNPs based on Lipids 9, 10, 11, and 15.

FIG. 7A depicts diameter (DLS, nm) of LNPs based on Lipid 31 to Lipid 34 in pH 7.4 HBS, pH 6.5 MBS, Post antibody (αCD3, hSP34) insertion and post freeze-thaw (−80° C.).

FIG. 7B depicts polydispersity (DLS) of LNPs based on Lipid 31 to Lipid 34 in pH 7.4 HBS, pH 6.5 MBS, Post antibody (αCD3, hSP34) insertion and post freeze-thaw (−80° C.).

FIG. 7C depicts charge (Zeta potential, DLS) of LNPs based on Lipid 31 to Lipid 34 in pH 5.5 MBS, pH 7.4 HBS.

FIG. 7D depicts % RNA recovery and dye accessible RNA in LNPs based on Lipid 31 to Lipid 34.

FIG. 8A depicts diameter (DLS, nm) of LNPs based on Lipids 1, 3, 4, 5, 9, and 15 in pH 7.4 HBS, pH 6.5 MBS, Post inserted with αCD8 antibody conjugates TRX-2 and T8.

FIG. 8B depicts polydispersity (DLS) of LNPs based on Lipids 1, 3, 4, 5, 9, and 15 in pH 7.4 HBS, pH 6.5 MBS, Post inserted with αCD8 antibody conjugates TRX-2 and T8.

FIG. 9A depicts diameter (DLS, nm) of LNPs based on Lipids 1, 8, 9, 10, 11, and 15 in pH 7.4 HBS, pH 6.5 MBS, Post inserted with αCD8 antibody conjugates TRX-2 and T8.

FIG. 9B depicts polydispersity (DLS) of LNPs based on Lipids 1, 8, 9, 10, 11, and 15 in pH 7.4 HBS, pH 6.5 MBS, Post inserted with αCD8 antibody conjugates TRX-2 and T8.

FIG. 10A depicts diameter (DLS, nm) of LNPs based on Lipid 3, 4, 33, and 34 in pH 7.4 HBS, pH 6.5 MBS, Post antibody (αCD3, hSP34) insertion and post freeze-thaw (−80° C.).

FIG. 10B depicts polydispersity (DLS) of LNPs based on Lipid 3, 4, 33, and 34 in pH 7.4 HBS, pH 6.5 MBS, Post antibody (αCD3, hSP34) insertion and post freeze-thaw (−80° C.).

FIG. 10C depicts charge (Zeta potential, DLS) of LNPs based on Lipid 3, 4, 33, and 34 in pH 5.5 MBS, pH7.4 HBS, pH6.5 MBS, post antibody (αCD3, hSP34) insertion and post freeze-thaw (−80° C.).

FIG. 10D depicts % RNA recovery and dye accessible RNA in LNPs based on Lipid 3, 4, 33, and 34.

FIG. 11A depicts GFP expression in primary human T-cells; transfected by αCD8 (hsp34) targeted LNPs based on ALC-0315, DLin-MC3-DMA, Lipid 3, Lipid 6, and Lipid 7, stored at 4° C.; % GFP+ T cells at 24 hours.

FIG. 11B depicts GFP expression in primary human T-cells; transfected by αCD3 (hsp34) targeted LNPs based on ALC-0315, DLin-MC3-DMA, Lipid 3, Lipid 6, and Lipid 7, after 1 freeze-thaw cycle (−80° C. storage); % GFP+ T cells at 24 hours.

FIG. 11C depicts GFP expression in primary human T-cells; transfected by αCD3 (hsp34) targeted LNPs based on ALC-0315, DLin-MC3-DMA, Lipid 3, Lipid 6, and Lipid 7, stored at 4° C.; GFP MFI in live T-cells at 24 hours.

FIG. 11D depicts GFP expression in primary human T-cells; transfected by αCD3 (hsp34) targeted LNPs based on ALC-0315, DLin-MC3-DMA, Lipid 3, Lipid 6, and Lipid 7, after 1 freeze-thaw cycle (−80° C. storage); GFP MFI in live T-cells at 24 hours.

FIG. 11E depicts % live T-cells transfected by αCD3 (hsp34) targeted LNPs based on ALC-0315, DLin-MC3-DMA, Lipid 3, Lipid 6, and Lipid 7, after 1 freeze-thaw cycle (−80° C. storage); % live T-cells at 24 hours.

FIG. 12A depicts GFP expression in primary human T-cells; transfected by αCD3 (hsp34) targeted LNPs based on SM-102, DLin-KC2-DMA, Lipid 3, Lipid 4 stored at 4° C.; % GFP+ T cells at 24 hours.

FIG. 12B depicts GFP expression in primary human T-cells; transfected by αCD3 (hsp34) targeted LNPs based on SM-102, DLin-KC2-DMA, Lipid 3, Lipid 4, after 1 freeze-thaw cycle (−80° C. storage); % GFP+ T cells at 24 hours.

FIG. 12C depicts GFP expression in primary human T-cells; transfected by αCD3 (hsp34) targeted LNPs based on SM-102, DLin-KC2-DMA, Lipid 3, Lipid 4, stored at 4° C.; GFP MFI in live T-cells at 24 hours.

FIG. 12D depicts GFP expression in primary human T-cells; transfected by αCD3 (hsp34) targeted LNPs based on SM-102, DLin-KC2-DMA, Lipid 3, Lipid 4, after 1 freeze-thaw cycle (−80° C. storage); GFP MFI in live T-cells at 24 hours.

FIG. 12E depicts % live T-cells transfected with by αCD3 (hsp34) targeted LNPs based on SM-102, DLin-KC2-DMA, Lipid 3, Lipid 4, after 1 freeze-thaw cycle (−80° C. storage); % live T-cells at 24 hours.

FIG. 13A depicts GFP expression in primary human T-cells; transfected by targeted LNPs based on DLin-KC2-DMA (−80° C. stored), Lipid 1 (4° C. stored), Lipid 3 (4° C. stored), and Lipid 5 (4° C. stored); % GFP+ T cells.

FIG. 13B depicts GFP expression in primary human T-cells; transfected by targeted LNPs based on DLin-KC2-DMA, Lipid 1, Lipid 3, and Lipid 5 after freeze-thaw cycle (−80° C. storage); % GFP+ T cells.

FIG. 13C depicts GFP expression in primary human T-cells; transfected by targeted LNPs based on DLin-KC2-DMA (−80° C. stored), Lipid 1 (4° C. stored), Lipid 3 (4° C. stored), and Lipid 5 (4° C. stored), GFP MFI in live T-cells.

FIG. 13D depicts GFP expression in primary human T-cells; transfected by a targeted LNPs based on DLin-KC2-DMA, Lipid 1, Lipid 3, and Lipid 5 after freeze-thaw cycle (−80° C. storage); GFP MFI in live T-cells.

FIG. 13E depicts % live T-cells transfected with targeted LNPs based on DLin-KC2-DMA, Lipid 1, Lipid 3, and Lipid 5 stored at −80° C.

FIG. 14A depicts GFP expression in primary human T-cells; transfected by αCD3 (hSP34) targeted LNPs based on DLin-KC2-DMA, Lipid 1 (4° C. stored), Lipid 8 (4° C. stored), and Lipid 8 (−80° C. stored); % GFP+ T cells.

FIG. 14B depicts GFP expression in primary human T-cells; transfected by αCD3 (hSP34) targeted LNPs based on DLin-KC2-DMA, Lipid 1 (4° C. stored), Lipid 8 (4° C. stored), and Lipid 8 (−80° C. stored); GFP MFI in live T-cells.

FIG. 14C depicts % living cells with targeted LNPs based on DLin-KC2-DMA, Lipid 1 (4° C. stored), Lipid 8 (4° C. stored), and Lipid 8 (−80° C. stored).

FIG. 15A depicts GFP expression in primary human T-cells; transfected by αCD3 (hsp34) targeted LNPs based on DLin-KC2-DMA (−80° C. stored), Lipid 8 (4° C. stored), Lipid 9 (4° C. stored), and Lipid 10 (4° C. stored); % GFP+ T cells.

FIG. 15B depicts GFP expression in primary human T-cells; transfected by αCD3 (hsp34) targeted LNPs based on DLin-KC2-DMA (−80° C. stored), Lipid 8 (−80° C. stored) and Lipid 10 (−80° C. stored); % GFP+ T cells.

FIG. 15C depicts GFP expression in primary human T-cells; transfected by αCD3 (hsp34) targeted LNPs based on DLin-KC2-DMA (−80° C. stored), Lipid 8 (4° C. stored), Lipid 9 (4° C. stored), and Lipid 10 (4° C. stored); GFP MFI in live T-cells.

FIG. 15D depicts GFP expression in primary human T-cells; transfected by αCD3 (hsp34) targeted LNPs based on DLin-KC2-DMA (−80° C. stored), Lipid 8 (−80° C. stored) and Lipid 10 (−80° C. stored); GFP MFI in live T-cells.

FIG. 15E depicts % live T-cells transfected with αCD3 (hsp34) targeted LNPs based on DLin-KC2-DMA (−80° C. stored), Lipid 8 (4° C. stored), Lipid 9 (4° C. stored), and Lipid 10(4° C. stored); % live T-cells.

FIG. 15F depicts % live T-cells transfected with αCD3 (hsp34) targeted LNPs based on DLin-KC2-DMA (−80° C. stored), Lipid 8 (−80° C. stored) and Lipid 10 (−80° C. stored); % live T-cells.

FIG. 16A depicts GFP expression in primary human T-cells; transfected by αCD3 (hsp34) targeted LNPs based on DLin-KC2-DMA (−80° C. stored), Lipid 3 (4° C. stored), Lipid 4 (4° C. stored), Lipid 9 (4° C. stored), Lipid 15 (4° C. stored); % GFP+ T cells.

FIG. 16B depicts GFP expression in primary human T-cells; transfected by αCD3 (hsp34) targeted LNPs based on DLin-KC2-DMA (−80° C. stored), Lipid 3 (−80° C. stored), Lipid 4 (−80° C. stored), Lipid 9 (−80° C. stored), Lipid 15 (−80° C. stored); % GFP+ T cells.

FIG. 16C depicts GFP expression in primary human T-cells; transfected by αCD3 (hsp34) targeted LNPs based on DLin-KC2-DMA (−80° C. stored), Lipid 3 (4° C. stored), Lipid 4 (4° C. stored), Lipid 9 (4° C. stored), Lipid 15 (4° C. stored), GFP MFI in live T-cells.

FIG. 16D depicts GFP expression in primary human T-cells; transfected by αCD3 (hsp34) targeted LNPs based on DLin-KC2-DMA (−80° C. stored), Lipid 3 (−80° C. stored), Lipid 4 (−80° C. stored), Lipid 9 (−80° C. stored), Lipid 15 (−80° C. stored); GFP MFI in live T-cells.

FIG. 16E depicts % live T-cells transfected with αCD3 (hsp34) targeted LNPs based on DLin-KC2-DMA (−80° C. stored), Lipid 3 (−80° C. stored), Lipid 4 (−80° C. stored), Lipid 9 (−80° C. stored), and Lipid 15 (−80° C. stored).

FIG. 17A depicts GFP expression in primary human T-cells; transfected by αCD8 (TRX2) targeted LNPs based on Lipid 3, Lipid 4, Lipid 9, Lipid 15, and compared with the corresponding non-targeted parent LNPs; % GFP+ T cells.

FIG. 17B depicts GFP expression in primary human T-cells; transfected by αCD8 (TRX2) targeted LNPs based on Lipid 3, Lipid 4, Lipid 9, Lipid 15, and compared with the corresponding non-targeted parent LNPs; GFP MFI in live T-cells.

FIG. 17C depicts % +Dil T-cell with αCD8 (TRX2) targeted LNPs based on Lipid 3, Lipid 4, Lipid 9, Lipid 15, and compared with the corresponding non-targeted parent LNPs.

FIG. 17D depicts Dil MFI in live T-cells with αCD8 (TRX2) targeted LNPs based on Lipid 3, Lipid 4, Lipid 9, Lipid 15, and compared with the corresponding non-targeted parent LNPs.

FIG. 17E depicts % live T-cells transfected with αCD8 (TRX2) targeted LNPs based on Lipid 3, Lipid 4, Lipid 9, Lipid 15, and compared with the corresponding non-targeted parent LNPs.

FIG. 18A depicts GFP expression in primary human T-cells; transfected by αCD8 (T8) targeted LNPs based on Lipid 3, Lipid 4, Lipid 9, Lipid 15, and compared with the corresponding non-targeted parent LNPs; % GFP+ T cells.

FIG. 18B depicts GFP expression in primary human T-cells; transfected by αCD8 (T8) targeted LNPs based on Lipid 3, Lipid 4, Lipid 9, Lipid 15, and compared with the corresponding non-targeted parent LNPs; GFP MFI in live T-cells.

FIG. 18C depicts % +Dil T-cell with αCD8 (T8) targeted LNPs based on Lipid 3, Lipid 4, Lipid 9, Lipid 15, and compared with the corresponding non-targeted parent LNPs.

FIG. 18D depicts Dil MFI in live T-cells with αCD8 (T8) targeted LNPs based on Lipid 3, Lipid 4, Lipid 9, Lipid 15, and compared with the corresponding non-targeted parent LNPs.

FIG. 18E depicts % live T-cells transfected with αCD8 (T8) targeted LNPs based on Lipid 3, Lipid 4, Lipid 9, Lipid 15, and compared with the corresponding non-targeted parent LNPs.

FIG. 19A depicts GFP expression in primary human T-cells; transfected by αCD3 (hSP34) targeted LNPs based on DLin-KC2-DMA, Lipid 2, Lipid 3, Lipid 31, and Lipid 32; stored at 4° C.; % GFP+ T cells.

FIG. 19B depicts GFP expression in primary human T-cells; transfected by αCD3 (hSP34) targeted LNPs based on DLin-KC2-DMA, Lipid 2, Lipid 3, Lipid 31, and Lipid 32; stored at 4° C.; GFP MFI in live T-cells.

FIG. 19C depicts % living T-cells with αCD3 (hSP34) targeted LNPs based on DLin-KC2-DMA, Lipid 2, Lipid 3, Lipid 31, and Lipid 32; stored at 4° C.

FIG. 20A depicts GFP expression in primary human T-cells; transfected with αCD3 (hSP34) targeted LNPs based on DLin-KC2-DMA (−80° C. stored), Lipid 3 (4° C. stored), Lipid 33 (4° C. stored), Lipid 34 (4° C. stored), or transfected with αCD8 (muOKT8) targeted LNPs based on Lipid 33 (4° C. stored) Lipid 34 (4° C. stored); % GFP+ T cells.

FIG. 20B depicts GFP expression in primary human T-cells; transfected with αCD3 (hSP34) targeted LNPs based on DLin-KC2-DMA (−80° C. stored), Lipid 3 (−80° C. stored), Lipid 33 (−80° C. stored), Lipid 34 (−80° C. stored); % GFP+ T cells.

FIG. 20C depicts GFP expression in primary human T-cells; transfected with αCD3 (hSP34) targeted LNPs based on DLin-KC2-DMA (−80° C. stored), Lipid 3 (−80° C. stored), Lipid 33 (4° C. stored), Lipid 34 (4° C. stored), or transfected with αCD8 (muOKT8) targeted LNPs based on Lipid 33 (4° C. stored) Lipid 34 (4° C. stored); GFP MFI in live T-cells.

FIG. 20D depicts GFP expression in primary human T-cells; transfected with αCD3 (hSP34) targeted LNPs based on DLin-KC2-DMA (−80° C. stored), Lipid 3 (−80° C. stored), Lipid 33 (−80° C. stored), Lipid 34 (−80° C. stored); GFP MFI in live T-cells.

FIG. 20E depicts % live T-cells transfected with αCD3 (hSP34) targeted LNPs based on DLin-KC2-DMA (−80° C. stored), Lipid 3 (−80° C. stored), Lipid 33 (−80° C. stored), Lipid 34 (−80° C. stored), or transfected with αCD8 (muOKT8) targeted LNPs based on Lipid 33 (4° C. stored) and Lipid 34 (4° C. stored).

FIG. 21A depicts % αCD20 (TTR-023) CAR+ T-cells transfected by αCD3 (hSP34) targeted LNPs based on Lipid 3 (4° C. stored), Lipid 4 (4° C. stored), Lipid 9 (4° C. stored), and Lipid 33 (4° C. stored); as illustrated by % M1 value.

FIG. 21B depicts % αCD20 (TTR-023) CAR+ T-cells transfected by αCD3 (hSP34) targeted LNPs based on Lipid 3 (−80° C. stored), Lipid 4 (−80° C. stored), Lipid 9 (−80° C. stored), Lipid 33 (−80° C. stored); as illustrated by % M1 value.

FIG. 21C depicts % αCD20 (TTR-023) CAR MFI in T-cells transfected by αCD3 (hSP34) targeted LNPs based on Lipid 3 (4° C. stored), Lipid 4 (4° C. stored), Lipid 9 (4° C. stored), and Lipid 33 (4° C. stored).

FIG. 21D depicts % αCD3 (hSP34) CAR MFI in T-cells transfected by αCD3 (hSP34) targeted LNPs based on Lipid 3 (−80° C. stored), Lipid 4 (−80° C. stored), Lipid 9 (−80° C. stored), Lipid 33 (−80° C. stored).

FIG. 21E depicts % live T-cells transfected with αCD3 (hSP34) targeted LNPs based on Lipid 3 (4° C. stored), Lipid 4 (4° C. stored), Lipid 9 (4° C. stored), and Lipid 33 (4° C. stored).

FIG. 21F depicts % live T-cells transfected with αCD3 (hSP34) targeted LNPs based on Lipid 3 (−80° C. stored), Lipid 4 (−80° C. stored), Lipid 9 (−80° C. stored), Lipid 33 (−80° C. stored).

FIG. 22A depicts % αCD20 (TTR-023) CAR+ T-cells (CD8 population) with αCD8 (T8) targeted LNPs based on Lipid 3 (4° C. stored), Lipid 4 (4° C. stored), Lipid 9 (4° C. stored), Lipid 33 (4° C. stored), as illustrated by CD4− % M1 value.

FIG. 22B depicts αCD20 (TTR-023) CAR MFI in T-cells (CD8 population) with αCD8 (T8) targeted LNPs based on Lipid 3 (4° C. stored), Lipid 4 (4° C. stored), Lipid 9 (4° C. stored), Lipid 33 (4° C. stored), as illustrated by CD4− M1 MFI value.

FIG. 22C depicts αCD20 (TTR-023) CAR level in CD4+ T-cells transfected with αCD8 (T8) targeted LNPs based on Lipid 3 (4° C. stored), Lipid 4 (4° C. stored), Lipid 9 (4° C. stored), Lipid 33 (4° C. stored); as illustrated by the M1% value.

FIG. 22D depicts αCD20 (TTR-023) CAR level in CD4+ T-cells transfected with αCD8 (T8) targeted LNPs based on DLin-KC2-DMA (−80° C. stored), Lipid 3 (−80° C. stored), Lipid 33 (−80° C. stored), Lipid 34 (−80° C. stored); as illustrated by the M1 MFI value.

FIG. 22E depicts % live T-cells (CD4/CD8 populations) transfected with αCD8 (T8) targeted LNPs based on Lipid 3 (4° C. stored), Lipid 4 (4° C. stored), Lipid 9 (4° C. stored), Lipid 33 (4° C. stored).

FIG. 23A depicts % αCD20 (TTR-023) CAR+ T-cells (CD8 population) transfected with αCD8 (T8) targeted LNPs based on Lipid 3 (−80° C. stored), Lipid 4 (−80° C. stored), Lipid 9 (−80° C. stored), Lipid 33 (−80° C. stored) after one Freeze-Thaw cycle, as illustrated by CD4-% M1 value.

FIG. 23B depicts αCD20 (TTR-023) CAR MFI in T-cells (CD8 population) transfected with αCD8 (T8) targeted LNPs based on Lipid 3 (−80° C. stored), Lipid 4 (−80° C. stored), Lipid 9 (−80° C. stored), Lipid 33 (−80° C. stored) after one Freeze-Thaw cycle, as illustrated by CD4−M1 MFI value.

FIG. 23C depicts αCD20 (TTR-023) CAR level in CD4+ T-cells transfected with αCD8 (T8) targeted LNPs based on Lipid 3 (−80° C. stored), Lipid 4 (−80° C. stored), Lipid 9 (−80° C. stored), Lipid 33 (−80° C. stored); as illustrated by CD4+% M1 value.

FIG. 23D depicts αCD20 (TTR-023) CAR level in CD4+ T-cells transfected with αCD8 (T8) targeted LNPs based on Lipid 3 (−80° C. stored), Lipid 4 (−80° C. stored), Lipid 9 (−80° C. stored), Lipid 33 (−80° C. stored); as illustrated by CD4+M1 MFI value.

FIG. 23E depicts % live T-cells transfected with αCD8 (T8) targeted LNPs based on Lipid 3 (−80° C. stored), Lipid 4 (−80° C. stored), Lipid 9 (−80° C. stored), Lipid 33 (−80° C. stored).

FIG. 24A depicts GFP expression in CD8+ T-cells transfected with αCD3 (hSP34) targeted or αCD8 (TRX2) targeted LNPs based on Lipid 9, Lipid 15, or DLin-KC3-DMA compared to vector control (mutOKT8) and un-transfected; as illustrated by % GFP+ T cells.

FIG. 24B depicts GFP expression in CD8+ T-cells transfected with αCD3 (hSP34) targeted or αCD8 (TRX2) targeted LNPs based on Lipid 9, Lipid 15, or DLin-KC3-DMA compared to vector control (mutOKT8) and un-transfected; as illustrated by GFP MFI.

FIG. 24C depicts GFP expression in CD4+ T-cells transfected with αCD3 (hSP34) targeted or αCD8 (TRX2) targeted LNPs based on Lipid 9, Lipid 15, or DLin-KC3-DMA compared to vector control (mutOKT8) and un-transfected; as illustrated by % GFP+ T cells.

FIG. 24D depicts GFP expression in CD4+ T-cells transfected with αCD3 (hSP34) targeted or αCD8 (TRX2) targeted LNPs based on Lipid 9, Lipid 15, or DLin-KC3-DMA compared to vector control (mutOKT8) and un-transfected; as illustrated by GFP MFI.

FIG. 24E depicts % Dil+CD8+ T-cells transfected with αCD3 (hSP34) targeted or αCD8 (TRX2) targeted LNPs based on Lipid 9, Lipid 15, or DLin-KC3-DMA compared to vector control (mutOKT8) and un-transfected; as illustrated by % Dil+ T-cells.

FIG. 24F depicts Dil MFI in CD8+ T-cells transfected with αCD3 (hSP34) targeted or αCD8 (TRX2) targeted LNPs based on Lipid 9, Lipid 15, or DLin-KC3-DMA compared to vector control (mutOKT8) and un-transfected; as illustrated by Dil MFI.

FIG. 24G depicts % Dil+CD4+ T-cells transfected with αCD3 (hSP34) targeted or αCD8 (TRX2) targeted LNPs based on Lipid 9, Lipid 15, or DLin-KC3-DMA compared to vector control (mutOKT8) and un-transfected; as illustrated by % Dil+ T-cells.

FIG. 24H depicts Dil MFI in CD4+ T-cells transfected with αCD3 (hSP34) targeted or αCD8 (TRX2) targeted LNPs based on Lipid 9, Lipid 15, or DLin-KC3-DMA compared to vector control (mutOKT8) and un-transfected; as illustrated by Dil MFI.

FIG. 25A depicts GFP expression in NK cells in whole blood samples transfected with αCD3 (hSP34) targeted or αCD8 (TRX2) targeted LNPs based on Lipid 9, Lipid 15, or DLin-KC3-DMA compared to non-binding control (mutOKT8) and un-transfected; as illustrated by % GFP+NK cells.

FIG. 25B depicts GFP expression in NK cells in whole blood samples transfected with αCD3 (hSP34) targeted or αCD8 (TRX2) targeted LNPs based on Lipid 9, Lipid 15, or DLin-KC3-DMA compared to non-binding control (mutOKT8) and un-transfected; as illustrated by GFP MFI.

FIG. 25C depicts GFP expression in granulocytes in whole blood samples transfected with αCD3 (hSP34) targeted or αCD8 (TRX2) targeted LNPs based on Lipid 9, Lipid 15, or DLin-KC3-DMA compared to non-binding control (mutOKT8) and un-transfected; as illustrated by % GFP+ granulocytes.

FIG. 25D depicts GFP expression in granulocytes in whole blood samples transfected with αCD3 (hSP34) targeted or αCD8 (TRX2) targeted LNPs based on Lipid 9, Lipid 15, or DLin-KC3-DMA compared to non-binding control (mutOKT8) and un-transfected; as illustrated by GFP MFI.

FIG. 25E depicts GFP expression in B cells in whole blood samples transfected with αCD3 (hSP34) targeted or αCD8 (TRX2) targeted LNPs based on Lipid 9, Lipid 15, or DLin-KC3-DMA compared to non-binding control (mutOKT8) and un-transfected; as illustrated by % GFP+ B cells.

FIG. 25F depicts GFP expression in B cells in whole blood samples transfected with αCD3 (hSP34) targeted or αCD8 (TRX2) targeted LNPs based on Lipid 9, Lipid 15, or DLin-KC3-DMA compared to non-binding control (mutOKT8) and un-transfected; as illustrated by GFP MFI.

FIG. 26A depicts LNP binding to NK cells in whole blood samples transfected with αCD3 (hSP34) targeted or αCD8 (TRX2) targeted LNPs based on Lipid 9, Lipid 15, or DLin-KC3-DMA compared to non-binding control (mutOKT8) and un-transfected; as illustrated by % Dil+NK cells.

FIG. 26B depicts LNP binding to NK cells in whole blood samples transfected with αCD3 (hSP34) targeted or αCD8 (TRX2) targeted LNPs based on Lipid 9, Lipid 15, or DLin-KC3-DMA compared to non-binding control (mutOKT8) and un-transfected; as illustrated by Dil MFI.

FIG. 26C depicts LNP binding to granulocytes in whole blood samples transfected with αCD3 (hSP34) targeted or αCD8 (TRX2) targeted LNPs based on Lipid 9, Lipid 15, or DLin-KC3-DMA compared to non-binding control (mutOKT8) and un-transfected; as illustrated by % Dil+ granulocytes.

FIG. 26D depicts LNP binding to granulocytes in whole blood samples transfected with αCD3 (hSP34) targeted or αCD8 (TRX2) targeted LNPs based on Lipid 9, Lipid 15, or DLin-KC3-DMA compared to non-binding control (mutOKT8) and un-transfected; as illustrated by Dil MFI.

FIG. 26E depicts LNP binding to B cells in whole blood samples transfected with αCD3 (hSP34) targeted or αCD8 (TRX2) targeted LNPs based on Lipid 9, Lipid 15, or DLin-KC3-DMA compared to non-binding control (mutOKT8) and un-transfected; as illustrated by % Dil+ B cells.

FIG. 26F depicts LNP binding to B cells in whole blood samples transfected with αCD3 (hSP34) targeted or αCD8 (TRX2) targeted LNPs based on Lipid 9, Lipid 15, or DLin-KC3-DMA compared to non-binding control (mutOKT8) and un-transfected; as illustrated by Dil MFI.

FIG. 27A depicts % live T-cells 24 hours after being transfected with αCD8 (TRX2) targeted LNPs expressing αCD20 (TTR-023) CAR or mCherry based on Lipid 9 or DLin-KC3-DMA.

FIG. 27B depicts % of CD8 (CD4−) T-cells expressing M1 (TRR-023) CAR after being transfected with αCD8 (TRX2) targeted LNPs expressing αCD20 (TTR-023) CAR or mCherry based on Lipid 9 or DLin-KC3-DMA.

FIG. 27C depicts M1 (TTR-023) expression Mean Fluorescence Intensity (MFI) in CD8 (CD4−) T-cells transfected with αCD8 (TRX2) targeted LNPs expressing αCD20 (TTR-023) CAR or mCherry based on Lipid 9 or DLin-KC3-DMA.

FIG. 27D depicts % of CD8 (CD4−) T-cells with mCherry expression after being transfected with αCD8 (TRX2) targeted LNPs expressing αCD20 (TTR-023) CAR or mCherry based on Lipid 9 or DLin-KC3-DMA.

FIG. 27E depicts mCherry expression Mean Fluorescence Intensity (MFI) in CD8 (CD4−) T-cells transfected with αCD8 (TRX2) targeted LNPs expressing αCD20 (TTR-023) CAR or mCherry based on Lipid 9 or DLin-KC3-DMA.

FIG. 27F depicts % of CD4+ T-cells with M1 (TTR-023) CAR expression after being transfected with αCD8 (TRX2) targeted LNPs expressing αCD20 (TTR-023) CAR or mCherry based on Lipid 9 or DLin-KC3-DMA.

FIG. 27G depicts M1 (TTR-023) expression Mean Fluorescence Intensity (MFI) in CD4+ T-cells after being transfected with αCD8 (TRX2) targeted LNPs expressing αCD20 (TTR-023) CAR or mCherry based on Lipid 9 or DLin-KC3-DMA.

FIG. 27H depicts % of CD4+ T-cells with mCherry expression after being transfected with αCD8 (TRX2) targeted LNPs expressing αCD20 (TTR-023) CAR or mCherry based on Lipid 9 or DLin-KC3-DMA.

FIG. 27I depicts % dead Raji cells in Raji (B-cell) co-culture experiment with CAR-T generated using αCD8 (TRX2) targeted LNPs expressing αCD20 (TTR-023) CAR or mCherry based on Lipid 9 or DLin-KC3-DMA.

FIG. 28A depicts % of dead Raji cells in Raji (B-cell) co-culture experiment with CAR-T cells generated using αCD8 (TRX2) targeted LNPs expressing αCD20 (TTR-023) CAR or mCherry based on Lipid 9 or DLin-KC3-DMA, with an effector:target ratio of 1:1, 4:1, and 8:1.

FIG. 28B depicts % of live CD8 (CD4−) T-cells in Raji (B-cell) co-culture experiment with CAR-T cells generated using αCD8 (TRX2) targeted LNPs expressing αCD20 (TTR-023) CAR or mCherry based on Lipid 9 or DLin-KC3-DMA, with an effector:target ratio of 1:1, 4:1, and 8:1.

FIG. 28C depicts % of live CD4+ T-cells in Raji (B-cell) co-culture experiment with CAR-T cells generated using αCD8 (TRX2) targeted LNPs expressing αCD20 (TTR-023) CAR or mCherry based on Lipid 9 or DLin-KC3-DMA, with an effector:target ratio of 1:1, 4:1, and 8:1.

FIG. 29A depicts % live T-cells 24 hours after being transfected with αCD8 (TRX2) targeted LNPs expressing αCD20 (TTR-023) CAR or mCherry based on Lipid 15 or DLin-KC3-DMA.

FIG. 29B depicts % of CD8 (CD4−) T-cells expressing M1 (TRR-023 CAR) after being transfected with αCD8 (TRX2) targeted LNPs expressing αCD20 (TTR-023) CAR or mCherry based on Lipid 15 or DLin-KC3-DMA.

FIG. 29C d depicts M1 (TTR-023 CAR) expression Mean Fluorescence Intensity (MFI) in CD8 (CD4−) T-cells transfected with αCD8 (TRX2) targeted LNPs expressing αCD20 (TTR-023) CAR or mCherry based on Lipid 15 or DLin-KC3-DMA.

FIG. 29D depicts % of CD8 (CD4−) T-cells with mCherry expression after being transfected with αCD8 (TRX2) targeted LNPs expressing αCD20 (TTR-023) CAR or mCherry based on Lipid 15 or DLin-KC3-DMA.

FIG. 29E depicts mCherry expression Mean Fluorescence Intensity (MFI) in CD8 (CD4−) T-cells transfected with αCD8 (TRX2) targeted LNPs expressing αCD20 (TTR-023) CAR or mCherry based on Lipid 15 or DLin-KC3-DMA.

FIG. 30A depicts % of dead Raji cells in Raji (B-cell) co-culture experiment with CAR-T cells generated using αCD8 (TRX2) targeted LNPs expressing αCD20 (TTR-023) CAR or mCherry based on Lipid 15 or DLin-KC3-DMA, with an effector:target ratio of 0.31:1, 1:1, 3.16:1, 10:1, and 31.6:1.

FIG. 30B depicts % of live CD8 (CD4−) T-cells in Raji (B-cell) co-culture experiment with CAR-T cells generated using αCD8 (TRX2) targeted LNPs expressing αCD20 (TTR-023) CAR or mCherry based on Lipid 15 or DLin-KC3-DMA, with an effector:target ratio of 0.31:1, 1:1, 3.16:1, 10:1, and 31.6:1.

FIG. 30C depicts % of live CD4+ T-cells in Raji (B-cell) co-culture experiment with CAR-T cells generated using αCD8 (TRX2) targeted LNPs expressing αCD20 (TTR-023) CAR or mCherry based on Lipid 15 or DLin-KC3-DMA, with an effector:target ratio of 0.31:1, 1:1, 3.16:1, 10:1, and 31.6:1.

FIG. 31 depicts structures of various Fab, VHH (Nb), ScFv, Fab-ScFv and Fab-V_{HH} hybrids.

FIG. 32A depicts GFP expression in T-cells transfected with αCD3 targeted LNPs based on Lipid 9, Lipid 10, Lipid 13, Lipid 15, or DLin-KC2-DMA, stored at either 4° C. or post freeze-thaw (−80° C. stored), as illustrated by % GFP+ T cells.

FIG. 32B depicts GFP expression in T-cells transfected with αCD3 targeted LNPs based on Lipid 9, Lipid 10, Lipid 13, Lipid 15, or DLin-KC2-DMA, stored at either 4° C. or post freeze-thaw (−80° C. stored), as illustrated by GFP MFI.

FIG. 32C depicts % Dil+ T-cells transfected with αCD3 targeted LNPs based on Lipid 9, Lipid 10, Lipid 13, Lipid 15, or DLin-KC2-DMA, stored at either 4° C. or post freeze-thaw (−80° C. stored), as illustrated by % Dil+ T-cells.

FIG. 32D depicts Dil MFI in live T-cells transfected with αCD3 targeted LNPs based on Lipid 9, Lipid 10, Lipid 13, Lipid 15, or DLin-KC2-DMA, stored at either 4° C. or post freeze-thaw (−80° C. stored), as illustrated by Dil % MFI.

FIG. 32E depicts % live T-cells transfected with αCD3 targeted LNPs based on Lipid 9, Lipid 10, Lipid 13, Lipid 15, or DLin-KC2-DMA, stored at either 4° C. or post freeze-thaw (−80° C. stored).

FIG. 33A to FIG. 33C depict % GFP+ T-cells (CD4 and CD8 populations) in Blood (FIG. 33A), Spleen (FIG. 33B), and Liver (FIG. 33C) samples (analyzed for additional cell types of interest per legend) at 24 hours post injection of GFP RNA using Lipid 15, DLin-KC3-DMA, and Lipid 9 LNP formulations and α-CD8 targeting with TRX-2 antibody.

FIG. 34A to FIG. 34C depict % DiI+ T-cells (CD4 and CD8 populations) in Blood (FIG. 34A), Spleen (FIG. 34B), and Liver (FIG. 34C) samples (analyzed for additional cell types of interest per legend) at 24 hours post injection of GFP RNA using Lipid 15 (DiI-dye labelled), DLin-KC3-DMA (No DiI-dye label used), and Lipid 9 LNP (DiI-dye labelled) formulations and α-CD8 targeting with TRX-2 antibody.

FIG. 35A depicts % GFP expression in CD8 T-cells with α-CD2, α-CD4, α-CD7, α-CD28, α-TCR and non-binding (mutated OKT8) targeted LNPs in Lipid 15 along with α-CD8 and α-CD3 Target.

FIG. 35B depicts GFP expression Mean Fluorescence Intensity (MFI) in CD8 T-cells with α-CD2, α-CD4, α-CD7, α-CD28, α-TCR and non-binding (mutated OKT8) targeted LNPs in Lipid 15 along with α-CD8 and α-CD3 Target.

FIG. 35C depicts % GFP expression in CD8 T-cells with α-CD2, α-CD4, α-CD7, α-CD28, α-TCR and non-binding (mutated OKT8) targeted LNPs in Lipid DLin-KC3-DMA along with α-CD8 and α-CD3 Target.

FIG. 35D depicts GFP expression Mean Fluorescence Intensity (MFI) in CD8 T-cells with α-CD2, α-CD4, α-CD7, α-CD28, α-TCR and non-binding (mutated OKT8) targeted LNPs in Lipid DLin-KC3-DMA along with α-CD8 and α-CD3 Target.

FIG. 35E depicts % GFP expression in CD4 T-cells with α-CD2, α-CD4, α-CD7, α-CD28, α-TCR and non-binding (mutated OKT8) targeted LNPs in Lipid 15 along with α-CD8 and α-CD3 Target.

FIG. 35F depicts GFP expression Mean Fluorescence Intensity (MFI) in CD4 T-cells with α-CD2, α-CD4, α-CD7, α-CD28, α-TCR and non-binding (mutated OKT8) targeted LNPs in Lipid 15 along with α-CD8 and α-CD3 Target.

FIG. 35G depicts % GFP expression in CD4 T-cells with α-CD2, α-CD4, α-CD7, α-CD28, α-TCR and non-binding (mutated OKT8) targeted LNPs in Lipid DLin-KC3-DMA along with α-CD8 and α-CD3 Target.

FIG. 35H depicts GFP expression Mean Fluorescence Intensity (MFI) in CD4 T-cells with α-CD2, α-CD4, α-CD7, α-CD28, α-TCR and non-binding (mutated OKT8) targeted LNPs in Lipid DLin-KC3-DMA along with α-CD8 and α-CD3 Target.

FIG. 36A depicts % DiI+ in CD8 T-cells with α-CD2, α-CD4, α-CD7, α-CD28, α-TCR and non-binding (mutated OKT8) targeted LNPs in Lipid 15 along with α-CD8 and α-CD3 Target.

FIG. 36B depicts DiI MFI in CD8 T-cells with α-CD2, α-CD4, α-CD7, α-CD28, α-TCR and non-binding (mutated OKT8) targeted LNPs in Lipid 15 along with α-CD8 and α-CD3 Target.

FIG. 36C depicts % DiI+ in CD8 T-cells with α-CD2, α-CD4, α-CD7, α-CD28, α-TCR and non-binding (mutated OKT8) targeted LNPs in Lipid DLin-KC3-DMA along with α-CD8 and α-CD3 Target.

FIG. 36D depicts % DiI+ in CD8 T-cells with α-CD2, α-CD4, α-CD7, α-CD28, α-TCR and non-binding (mutated OKT8) targeted LNPs in Lipid DLin-KC3-DMA along with α-CD8 and α-CD3 Target.

FIG. 36E depicts % DiI+ in CD4 T-cells with α-CD2, α-CD4, α-CD7, α-CD28, α-TCR and non-binding (mutated OKT8) targeted LNPs in Lipid 15 along with α-CD8 and α-CD3 Target.

FIG. 36F depicts DiI MFI in CD4 T-cells with α-CD2, α-CD4, α-CD7, α-CD28, α-TCR and non-binding (mutated OKT8) targeted LNPs in Lipid 15 along with α-CD8 and α-CD3 Target.

FIG. 36G depicts % DiI+ in CD4 T-cells with α-CD2, α-CD4, α-CD7, α-CD28, α-TCR and non-binding (mutated OKT8) targeted LNPs in Lipid DLin-KC3-DMA along with α-CD8 and α-CD3 Target.

FIG. 36H depicts DiI MFI in CD4 T-cells with α-CD2, α-CD4, α-CD7, α-CD28, α-TCR and non-binding (mutated OKT8) targeted LNPs in Lipid DLin-KC3-DMA along with α-CD8 and α-CD3 Target.

FIG. 37A depicts Lipids 10, 15, 16, 24A, 26, and ALC-0315 Lipid targeted LNP (αCD8) diameters (DLS, nm) pre and post freeze-thaw.

FIG. 37B depicts Lipids 10, 15, 16, 24A, 26, and ALC-0315 Lipid targeted LNP (αCD8) polydispersity (DLS) pre and post freeze-thaw.

FIG. 37C depicts Lipids 10, 15, 16, 24A, 26, and ALC-0315 Lipid LNP Zeta Potential (mV) in pH 5.5 MES and pH 7.4 HBS.

FIG. 37D depicts Lipids 10, 15, 16, 24A, 26, and ALC-0315 Lipid Total RNA content (ug/mL) and % Dye accessible RNA.

FIG. 38A depicts % GFP+ T-cells in Lipid 10, 15, 16, 24A, 26, and ALC-0315 LNP transfections.

FIG. 38B depicts GFP-MFI of T-cells in Lipid 10, 15, 16, 24A, 26, and ALC-0315 LNP transfections.

FIG. 38C depicts % DiI+ T-cells in Lipid 10, 15, 16, 24A, 26, and ALC-0315 LNP transfections.

FIG. 38D depicts DiI-MFI of T-cells in Lipid 10, 15, 16, 24A, 26, and ALC-0315 LNP transfections.

FIG. 38E depicts % Live T-cells in Lipid 10, 15, 16, 24A, 26, and ALC-0315 LNP transfections.

FIG. 39A depicts charge (Zeta potential, DLS) of GFP and BiTE LNPs based on DLin-KC3-DMA in pH 5.5 MBS, pH 7.4 HBS before antibody insertion.

FIG. 39B depicts diameter (DLS, nm) of GFP and BiTE LNPs based on DLin-KC3-DMA and polydispersity (DLS) of GFP and BiTE LNPs based on DLin-KC3-DMA before antibody insertion.

FIG. 39C depicts % RNA recovery and dye accessible RNA in GFP and BiTE LNPs based on DLin-KC3-DMA before antibody insertion.

FIG. 39D depicts diameter Z-average size (DLS, nm) of BiTE LNPs based on DLin-KC3-DMA and polydispersity (DLS) of BiTE LNPs based on DLin-KC3-DMA post antibody (αCD3, 500A2 and αCD8, YTS156.7.7) insertion.

FIG. 40A depicts % live primary murine T-cells transfected by electroporation, αCD4 (GK1.5), αCD3 (500A2), and/or αCD8 (YTS156.7.7) targeted LNPs based on DLin-KC3-DMA; % live T-cells at 24 hours.

FIG. 40B depicts DiI LNP association in CD4− primary murine T-cells transfected by electroporation, αCD4 (GK1.5), αCD3 (500A2), and/or αCD8 (YTS156.7.7) targeted LNPs based on DLin-KC3-DMA; % DiI+ live T-cells at 24 hours.

FIG. 40C depicts DiI LNP association in CD8− primary murine T-cells transfected by electroporation, αCD4 (GK1.5), αCD3 (500A2), and/or αCD8 (YTS156.7.7) targeted LNPs based on DLin-KC3-DMA; % DiI+ live T-cells at 24 hours.

FIG. 40D depicts DiI LNP association in CD4− primary murine T-cells transfected by electroporation, αCD4 (GK1.5), αCD3 (500A2), and/or αCD8 (YTS156.7.7) targeted LNPs based on DLin-KC3-DMA; DiI MFI in live T-cells at 24 hours.

FIG. 40E depicts DiI LNP association in CD8− primary murine T-cells transfected by electroporation, αCD4 (GK1.5), αCD3 (500A2), and/or αCD8 (YTS156.7.7) targeted LNPs based on DLin-KC3-DMA; DiI MFI in live T-cells at 24 hours.

FIG. 40F depicts GFP LNP transfection in CD4− primary murine T-cells transfected by electroporation, αCD4 (GK1.5), αCD3 (500A2), and/or αCD8 (YTS156.7.7) targeted LNPs based on DLin-KC3-DMA; % GFP+ live T-cells at 24 hours.

FIG. 40G depicts GFP LNP transfection in CD8− primary murine T-cells transfected by electroporation, αCD4 (GK1.5), αCD3 (500A2), and/or αCD8 (YTS156.7.7) targeted LNPs based on DLin-KC3-DMA; % GFP+ live T-cells at 24 hours.

FIG. 40H depicts GFP LNP transfection in CD4− primary murine T-cells transfected by electroporation, αCD4 (GK1.5), αCD3 (500A2), and/or αCD8 (YTS156.7.7) targeted LNPs based on DLin-KC3-DMA; GFP MFI in live T-cells at 24 hours.

FIG. 40I depicts GFP LNP transfection in CD8− primary murine T-cells transfected by electroporation, αCD4 (GK1.5), αCD3 (500A2), and/or αCD8 (YTS156.7.7) targeted LNPs based on DLin-KC3-DMA; GFP MFI in live T-cells at 24 hours.

FIG. 41A depicts DiI LNP association in primary murine T-cells transfected by αCD8 (2.43, YTS156.7.7, or YTS169.4.2.1) targeted LNPs (inserted at 5, 15, or 30 Fabs/LNP) based on DLin-KC3-DMA; % DiI+ live T-cells at 24 hours.

FIG. 41B depicts GFP LNP transfection in primary murine T-cells transfected by αCD8 (2.43, YTS156.7.7, or YTS169.4.2.1) targeted LNPs (inserted at 5, 15, or 30 Fabs/LNP) based on DLin-KC3-DMA; % GFP+ live T-cells at 24 hours.

FIG. 41C depicts DiI LNP association in primary murine T-cells transfected by αCD3 (2C11, 500A2, or KT3) or αTCR (H57) targeted LNPs (inserted at 5, 15, or 30 Fabs/LNP) based on DLin-KC3-DMA; % DiI+ live T-cells at 24 hours.

FIG. 41D depicts GFP LNP transfection in primary murine T-cells transfected by αCD3 (2C11, 500A2, or KT3) or αTCR (H57) targeted LNPs (inserted at 5, 15, or 30 Fabs/LNP) based on DLin-KC3-DMA; % GFP+ live T-cells at 24 hours.

FIG. 41E depicts DiI LNP association in primary murine T-cells transfected by αCD4 (GK1.5v1) targeted LNPs (inserted at 2.5, 5, 15, or 30 Fabs/LNP) based on DLin-KC3-DMA; % DiI+ live T-cells at 24 hours.

FIG. 41F depicts GFP LNP transfection in primary murine T-cells transfected by αCD4 (GK1.5v1) targeted LNPs (inserted at 2.5, 5, 15, or 30 Fabs/LNP) based on DLin-KC3-DMA; % GFP+ live T-cells at 24 hours.

FIG. 41G depicts % DiI in murine T-cells transfected with various α-CD3/α-CD8 targeted DLIN-KC3-DMA LNPs.

FIG. 41H depicts % GFP+ murine T-cells transfected with various α-CD3/α-CD8 targeted DLIN-KC3-DMA LNPs.

FIG. 42A depicts CD69 expression in primary murine T-cells transfected by αCD8 (YTS156.7.7) and/or αCD3 (500A2) targeted LNPs based on DLin-KC3-DMA; % CD69+ live T-cells at 24 hours.

FIG. 42B depicts IFN-gamma secretion from primary murine T-cells transfected by αCD8 (YTS156.7.7) and/or αCD3 (500A2) targeted LNPs based on DLin-KC3-DMA; Concentration (pg/mL) of IFN-gamma in supernatants at 24 hours.

FIG. 42C depicts TNF-alpha secretion from primary murine T-cells transfected by αCD8 (YTS156.7.7) and/or αCD3 (500A2) targeted LNPs based on DLin-KC3-DMA; Concentration (pg/mL) of TNF-alpha in supernatants at 24 hours.

FIG. 42D depicts phenotype of primary murine T-cells transfected by αCD8 (YTS156.7.7) and/or αCD3 (500A2) targeted LNPs based on DLin-KC3-DMA; CM, central memory; EM, effector memory; SCM, T memory stem cell-like; Cells were phenotyped 24 hours after transfection.

FIG. 42E depicts gene expression levels of genes associated with activation in primary murine T-cells transfected by αCD8 (YTS156.7.7) and/or αCD3 (500A2) targeted LNPs based on DLin-KC3-DMA; Gene expression assessed 24 hours after transfection.

FIG. 43A depicts DiI LNP association of primary murine T-cells in vivo treated with αCD4 (GK1.5v1), αCD8 (YTS156.7.7) and/or αCD3 (500A2) targeted LNPs based on DLin-KC3-DMA; % DiI+ of CD8+ T-cells in blood 24 hours after intravenous injection.

FIG. 43B depicts DiI LNP association of primary murine T-cells in vivo treated with αCD4 (GK1.5v1), αCD8 (YTS156.7.7) and/or αCD3 (500A2) targeted LNPs based on DLin-KC3-DMA; % DiI+ of CD4+ T-cells in blood 24 hours after intravenous injection.

FIG. 43C depicts mCherry LNP expression of primary murine T-cells in vivo treated with αCD4 (GK1.5v1), αCD8 (YTS156.7.7) and/or αCD3 (500A2) targeted LNPs based on DLin-KC3-DMA; % mCherry+ of CD8+ T-cells in blood 24 hours after intravenous injection.

FIG. 43D depicts mCherry LNP expression of primary murine T-cells in vivo treated with αCD4 (GK1.5v1), αCD8 (YTS156.7.7) and/or αCD3 (500A2) targeted LNPs based on DLin-KC3-DMA; % mCherry+ of CD4+ T-cells in blood 24 hours after intravenous injection.

FIG. 43E depicts DiI LNP association of primary murine T-cells in vivo treated with αCD4 (GK1.5v1), αCD8 (YTS156.7.7) and/or αCD3 (500A2) targeted LNPs based on DLin-KC3-DMA; % DiI+ of CD8+ T-cells in spleen 24 hours after intravenous injection.

FIG. 43F depicts DiI LNP association of primary murine T-cells in vivo treated with αCD4 (GK1.5v1), αCD8 (YTS156.7.7) and/or αCD3 (500A2) targeted LNPs based on DLin-KC3-DMA; % DiI+ of CD4+ T-cells in spleen 24 hours after intravenous injection.

FIG. 43G depicts mCherry LNP expression of primary murine T-cells in vivo treated with αCD4 (GK1.5v1), αCD8 (YTS156.7.7) and/or αCD3 (500A2) targeted LNPs based on DLin-KC3-DMA; % mCherry+ of CD8+ T-cells in spleen 24 hours after intravenous injection.

FIG. 43H depicts mCherry LNP expression of primary murine T-cells in vivo treated with αCD4 (GK1.5v1), αCD8 (YTS156.7.7) and/or αCD3 (500A2) targeted LNPs based on DLin-KC3-DMA; % mCherry+ of CD4+ T-cells in spleen 24 hours after intravenous injection.

FIG. 43I depicts DiI LNP association of primary murine T-cells in vivo treated with αCD4 (GK1.5v1), αCD8 (YTS156.7.7) and/or αCD3 (500A2) targeted LNPs based on DLin-KC3-DMA; % DiI+ of CD8+ T-cells in liver 24 hours after intravenous injection.

FIG. 43J depicts DiI LNP association of primary murine T-cells in vivo treated with αCD4 (GK1.5v1), αCD8 (YTS156.7.7) and/or αCD3 (500A2) targeted LNPs based on DLin-KC3-DMA; % DiI+ of CD4+ T-cells in liver 24 hours after intravenous injection.

FIG. 43K depicts mCherry LNP expression of primary murine T-cells in vivo treated with αCD4 (GK1.5v1), αCD8 (YTS156.7.7) and/or αCD3 (500A2) targeted LNPs based on DLin-KC3-DMA; % mCherry+ of CD8+ T-cells in liver 24 hours after intravenous injection.

FIG. 43L depicts mCherry LNP expression of primary murine T-cells in vivo treated with αCD4 (GK1.5v1), αCD8 (YTS156.7.7) and/or αCD3 (500A2) targeted LNPs based on DLin-KC3-DMA; % mCherry+ of CD4+ T-cells in liver 24 hours after intravenous injection.

FIG. 44A depicts % of dead CT26 cells in CT26 (EphA2+ cell line) co-culture experiment with BiTE secreting murine T-cells generated using αCD8 (YTS156.7.7) targeted LNPs secreting BiTE or expressing Fluc based on DLin-KC3-DMA, in a time course (0-120 hours).

FIG. 44B depicts % of dead CT26 cells in CT26 (EphA2+ cell line) co-culture experiment with BiTE secreting murine T-cells generated using αCD8 (YTS156.7.7) and αCD3 (500A2) targeted LNPs secreting BiTE or expressing Fluc based on DLin-KC3-DMA, in a time course (0-120 hours).

FIG. 44C depicts % of dead CT26 cells in CT26 (EphA2+ cell line) co-culture experiment with BiTE secreting murine T-cells generated using αCD4 (GK1.5v1) targeted LNPs secreting BiTE or expressing Fluc based on DLin-KC3-DMA, in a time course (0-120 hours).

FIG. 44D depicts % of dead CT26 cells in CT26 (EphA2+ cell line) co-culture experiment with BiTE secreting murine T-cells generated using αCD8 (YTS156.7.7) targeted LNPs secreting BiTE or expressing Flue based on DLin-KC3-DMA, in a time course (0-120 hours).r500A2, recombinant 500A2/EphA2 BiTE protein. Fluc, firefly luciferase.

FIG. 45A depicts efficacy study tumor inoculation and LNP dosing regimen.

FIG. 45B depicts efficacy (survival curve) of mice treated with αCD8 (YTS156.7.7) and αCD3 (500A2) targeted LNPs secreting BiTE based on DLin-KC3-DMA.

FIG. 45C depicts tumor growth of mice treated with αCD8 (YTS156.7.7) and αCD3 (500A2) targeted LNPs secreting BiTE based on DLin-KC3-DMA.

FIG. 45D depicts tumor growth of mice treated with αCD8 (YTS156.7.7) and αCD3 (500A2) targeted LNPs expressing non-BiTE protein based on DLin-KC3-DMA.

FIG. 45E depicts tumor growth of mice treated with recombinant BiTE protein.

FIG. 45F depicts tumor growth of mice treated with PD-1 Ab.

FIG. 45G depicts tumor growth of mice treated with vehicle.

FIG. 45H depicts relative body weight to baseline (%) of mice treated with αCD8 (YTS156.7.7) and αCD3 (500A2) targeted LNPs expressing BiTE protein based on DLin-KC3-DMA.

FIG. 46A depicts charge (Zeta potential, DLS) of mCherry and CAR LNPs based on Lipid 15 in pH 5.5 MBS, pH 7.4 HBS before antibody insertion.

FIG. 46B depicts diameter (DLS, nm) of mCherry and CAR LNPs based on Lipid 15 and polydispersity (DLS) of mCherry and CAR LNPs based on Lipid 15 before antibody insertion.

FIG. 46C depicts % RNA recovery and dye accessible RNA in mCherry and CAR LNPs based on Lipid 15 before antibody insertion.

FIG. 46D depicts diameter (DLS, nm) of mCherry and CAR LNPs based on Lipid 15 and polydispersity (DLS) of mCherry and CAR LNPs based on Lipid 15 post antibody (αCD8, TRX2 and/or αCD8, Ibalizumab) insertion.

FIG. 47A depicts % live CD3+, CD4+, and CD8+ primary human T-cells transfected by αCD4 (Ibalizumab) and/or αCD8 (TRX2) targeted LNPs based on Lipid 15; % live T-cells at 24 hours.

FIG. 47B depicts CAR expression in CD3+, CD4+, and CD8+ primary human T-cells transfected by αCD4 (Ibalizumab) and/or αCD8 (TRX2) targeted LNPs based on Lipid 15 24 hours post-transfection; CAR MFI in live T-cells at 24 hours.

FIG. 47C depicts CAR expression in CD3+, CD4+, and CD8+ primary human T-cells transfected by αCD4 (Ibalizumab) and/or αCD8 (TRX2) targeted LNPs based on Lipid 15 24 hours post-transfection; % CAR+ live T-cells at 24 hours.

FIG. 47D depicts mCherry expression in CD3+, CD4+, and CD8+ primary human T-cells transfected by αCD4 (Ibalizumab) and/or αCD8 (TRX2) targeted LNPs based on Lipid 15 24 hours post-transfection; mCherry MFI in live T-cells at 24 hours.

FIG. 47E depicts mCherry expression in CD3+, CD4+, and CD8+ primary human T-cells transfected by αCD4 (Ibalizumab) and/or αCD8 (TRX2) targeted LNPs based on Lipid 15 24 hours post-transfection; % mCherry+ in live T-cells at 24 hours.

### DETAILED DESCRIPTION

The invention provides ionizable cationic lipids, lipid-immune cell targeting group conjugates, and lipid nanoparticle compositions comprising such ionizable cationic lipids and/or lipid-immune cell (e.g., T-cell) targeting group conjugates, medical kits containing such lipids and/or conjugates, and methods of making and using, such lipids and conjugates.

The practice of the present invention employs, unless otherwise indicated, conventional techniques of organic chemistry, pharmacology, cell biology, and biochemistry. Such techniques are explained in the literature, such as in “Comprehensive Organic Synthesis” (B. M. Trost & I. Fleming, eds., 1991-1992); “Current protocols in molecular biology” (F. M. Ausubel et al., eds., 1987, and periodic updates); and “Current protocols in immunology” (J. E. Coligan et al., eds., 1991), each of which is herein incorporated by reference in its entirety. Various aspects of the invention are set forth below in sections; however, aspects of the invention described in one particular section are not to be limited to any particular section.

### I. Definitions

To facilitate an understanding of the present invention, a number of terms and phrases are defined below.

Unless defined otherwise, all technical and scientific terms used herein have the same meaning as commonly understood by one of ordinary skill in the art to which this invention belongs. The abbreviations used herein have their conventional meaning within the chemical and biological arts. The chemical structures and formulae set forth herein should be construed according to the standard rules of chemical valency known in the chemical arts. In addition, when a chemical group is a diradical, for example, it is understood a that the chemical groups can be bonded to their adjacent atoms in the remainder of the structure in one or both orientations, for example, —OC(O)— is interchangeable with —C(O)O— or —OC(S)— is interchangeable with —C(S)O—.

The terms “a” and “an” as used herein mean “one or more” and include the plural unless the context is inappropriate. In some embodiments, “one or more” is 1 or 2. In some embodiments, “one or more” is 1, 2, or 3. In some embodiments, “one or more” is 1, 2, 3, or 4. In some embodiments, “one or more” is 1, 2, 3, 4, or 5. In some embodiments, “one or more” is 1, 2, 3, 4, 5, or more.

The term “alkyl” as used herein refers to a saturated straight or branched hydrocarbon, such as a straight or branched group of 1-12, 1-10, or 1-6 carbon atoms, referred to herein as C₁-C₁₂alkyl, C₁-C₁₀alkyl, or C₁-C₆alkyl, respectively. In some embodiments, alkyl is optionally substituted. Exemplary alkyl groups include, but are not limited to, methyl, ethyl, propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, hexyl, heptyl, octyl, etc.

The term “alkylene” refers to a diradical of an alkyl group. In some embodiments, alkylene is optionally substituted. An exemplary alkylene group is —CH2CH2-.

The term “haloalkyl” refers to an alkyl group that is substituted with at least one halogen. For example, —CH₂F, —CHF₂, —CF₃, —CH₂CF₃, —CF₂CF₃, and the like.

“Alkenyl” refers to an unsaturated branched or straight-chain alkyl group having the indicated number of carbon atoms (e.g., 2 to 8, or 2 to 6 carbon atoms) and at least one carbon-carbon double bond. The group may be in either the cis or trans configuration (Z or E configuration) about the double bond(s). Alkenyl groups include, but are not limited to, ethenyl, propenyl (e.g., prop-1-en-1-yl, prop-1-en-2-yl, prop-2-en-1-yl (allyl), prop-2-en-2-yl), and butenyl (e.g., but-1-en-1-yl, but-1-en-2-yl, 2-methyl-prop-1-en-1-yl, but-2-en-1-yl, but-2-en-1-yl, but-2-en-2-yl, buta-1,3-dien-1-yl, buta-1,3-dien-2-yl).

“Alkynyl” refers to an unsaturated branched or straight-chain alkyl group having the indicated number of carbon atoms (e.g., 2 to 8 or 2 to 6 carbon atoms) and at least one carbon-carbon triple bond. Alkynyl groups include, but are not limited to, ethynyl, propynyl (e.g., prop-1-yn-1-yl, prop-2-yn-1-yl) and butynyl (e.g., but-1-yn-1-yl, but-1-yn-3-yl, but-3-yn-1-yl).

The term “oxo” is art-recognized and refers to a “═O” substituent. For example, a cyclopentane substituted with an oxo group is cyclopentanone.

The term “morpholinyl” refers to a substituent having the structure of:

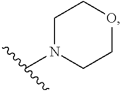 [SMILES: *N1CCOCC1]

which is optionally substituted.

The term “piperidinyl” refers to a substituent having a structure of:

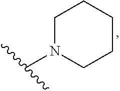 [SMILES: *N1CCCCC1]

which is optionally substituted.

In general, the term “substituted”, whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at each position. Combinations of substituents envisioned under this invention are preferably those that result in the formation of stable or chemically feasible compounds. In some embodiments, “optionally substituted” is equivalent to “unsubstituted or substituted.” In some embodiments, “optionally substituted” indicates that the designated atom or group is optionally substituted with one or more substituents independently selected from optional substituents provided herein. In some embodiments, optional substituent may be selected from the group consisting of: C₁₋₆alkyl, cyano, halogen, —O—C₁₋₆alkyl, C₁₋₆haloalkyl, C₃₋₇cycloalkyl, 3- to 7-membered heterocyclyl, 5- to 6-membered heteroaryl, and phenyl. In some embodiments, optional substituent is alkyl, cyano, halogen, halo, azide, aralkyl, alkenyl, alkynyl, cycloalkyl, hydroxyl, alkoxyl, amino, nitro, sulfhydryl, imino, amido, carboxylic acid, —C(O)alkyl, —CO₂alkyl, carbonyl, carboxyl, alkylthio, sulfonyl, sulfonamido, sulfonamide, ketone, aldehyde, ester, heterocyclyl, aryl, or heteroaryl. In some embodiments, optional substituent is —OR^{s1}, —NR^{s2}R^{s3}, —C(O)R^{s4}, —C(O)OR^{s5}, C(O)NR⁶R^{s7}, —OC(O)R^{s8}, —OC(O)OR^{s9}, —OC(O)NR^{s10}R¹¹, —NR^{s12}C(O)R^{s13} or —NR^{s14}C(O)OR^{s15}, wherein R^{s1}, R^{s2}, R^{s3}, R^{s4}, RSs, R^{s6}, R^{s7}, R^{s8}, R^{s9}, R^{s10}, R^{s11}, R^{s12}, R^{s13}, R^{s14}, and R^{s15} are each independently H, C₁₋₆ alkyl, C₃₋₁₀ cycloalkyl, C₆₋₁₄ aryl, 5- to 10-membered heteroaryl, or 3- to 10-membered heterocyclyl, each of which is optionally substituted.

The term “haloalkyl” refers to an alkyl group that is substituted with at least one halogen. For example, —CH₂F, —CHF₂, —CF₃, —CH₂CF₃, —CF₂CF₃, and the like.

The term “cycloalkyl” refers to a monovalent saturated cyclic, bicyclic, bridged cyclic (e.g., adamantyl), or spirocyclic hydrocarbon group of 3-12, 3-10, 3-8, 4-8, or 4-6 carbons, referred to herein, e.g., as “C₄₋₈cycloalkyl,” derived from a cycloalkane. In some embodiments, cycloalkyl is optionally substituted. Exemplary cycloalkyl groups include, but are not limited to, cyclohexanes, cyclopentanes, cyclobutanes and cyclopropanes. Unless specified otherwise, cycloalkyl groups are optionally substituted at one or more ring positions with, for example, alkanoyl, alkoxy, alkyl, haloalkyl, alkenyl, alkynyl, amido, amidino, amino, aryl, arylalkyl, azido, carbamate, carbonate, carboxy, cyano, cycloalkyl, ester, ether, formyl, halogen, haloalkyl, heteroaryl, heterocyclyl, hydroxyl, imino, ketone, nitro, phosphate, phosphonato, phosphinato, sulfate, sulfide, sulfonamido, sulfonyl or thiocarbonyl. In certain embodiments, the cycloalkyl group is not substituted, i.e., it is unsubstituted.

The terms “heterocyclyl” and “heterocyclic group” are art-recognized and refer to saturated, partially unsaturated, or aromatic 3- to 10-membered ring structures, alternatively 3- to 7-membered rings, whose ring structures include one to four heteroatoms, such as nitrogen, oxygen, and sulfur. In some embodiments, heterocyclyl is optionally substituted. The number of ring atoms in the heterocyclyl group can be specified using Cₓ-Cₓ nomenclature where x is an integer specifying the number of ring atoms. For example, a C₃-C₇heterocyclyl group refers to a saturated or partially unsaturated 3- to 7-membered ring structure containing one to four heteroatoms, such as nitrogen, oxygen, and sulfur. The designation “C₃-C₇” indicates that the heterocyclic ring contains a total of from 3 to 7 ring atoms, inclusive of any heteroatoms that occupy a ring atom position. One example of a C₃heterocyclyl is aziridinyl. Heterocycles may be, for example, mono-, bi-, or other multi-cyclic ring systems (e.g., fused, spiro, bridged bicyclic). A heterocycle may be fused to one or more aryl, partially unsaturated, or saturated rings. Heterocyclyl groups include, for example, biotinyl, chromenyl, dihydrofuryl, dihydroindolyl, dihydropyranyl, dihydrothienyl, dithiazolyl, homopiperidinyl, imidazolidinyl, isoquinolyl, isothiazolidinyl, isooxazolidinyl, morpholinyl, oxolanyl, oxazolidinyl, phenoxanthenyl, piperazinyl, piperidinyl, pyranyl, pyrazolidinyl, pyrazolinyl, pyridyl, pyrimidinyl, pyrrolidinyl, pyrrolidin-2-onyl, pyrrolinyl, tetrahydrofuryl, tetrahydroisoquinolyl, tetrahydropyranyl, tetrahydroquinolyl, thiazolidinyl, thiolanyl, thiomorpholinyl, thiopyranyl, xanthenyl, lactones, lactams such as azetidinones and pyrrolidinones, sultams, sultones, and the like. Unless specified otherwise, the heterocyclic ring is optionally substituted at one or more positions with substituents such as alkanoyl, alkoxy, alkyl, alkenyl, alkynyl, amido, amidino, amino, aryl, arylalkyl, azido, carbamate, carbonate, carboxy, cyano, cycloalkyl, ester, ether, formyl, halogen, haloalkyl, heteroaryl, heterocyclyl, hydroxyl, imino, ketone, nitro, oxo, phosphate, phosphonato, phosphinato, sulfate, sulfide, sulfonamido, sulfonyl and thiocarbonyl. In certain embodiments, the heterocyclyl group is not substituted, i.e., it is unsubstituted.

The term “aryl” is art-recognized and refers to a carbocyclic aromatic group. In some embodiments, aryl is optionally substituted. Representative aryl groups include phenyl, naphthyl, anthracenyl, and the like. The term “aryl” includes polycyclic ring systems having two or more carbocyclic rings in which two or more carbons are common to two adjoining rings (the rings are “fused rings”) wherein at least one of the rings is aromatic and, e.g., the other ring(s) may be cycloalkyls, cycloalkenyls, cycloalkynyls, and/or aryls. Unless specified otherwise, the aromatic ring may be substituted at one or more ring positions with, for example, halogen, azide, alkyl, aralkyl, alkenyl, alkynyl, cycloalkyl, hydroxyl, alkoxyl, amino, nitro, sulfhydryl, imino, amido, carboxylic acid, —C(O)alkyl, CO₂alkyl, carbonyl, carboxyl, alkylthio, sulfonyl, sulfonamido, sulfonamide, ketone, aldehyde, ester, heterocyclyl, aryl or heteroaryl moieties, —CF₃, —CN, or the like. In certain embodiments, the aromatic ring is substituted at one or more ring positions with halogen, alkyl, hydroxyl, or alkoxyl. In certain other embodiments, the aromatic ring is not substituted, i.e., it is unsubstituted. In certain embodiments, the aryl group is a 6- to 10-membered ring structure. In some embodiments, the aryl group is a C₆-C₄ aryl.

The term “heteroaryl” is art-recognized and refers to aromatic groups that include at least one ring heteroatom. In some embodiments, heteroaryl is optionally substituted. In certain instances, a heteroaryl group contains 1, 2, 3, or 4 ring heteroatoms. Representative examples of heteroaryl groups include pyrrolyl, furanyl, thiophenyl, imidazolyl, oxazolyl, thiazolyl, triazolyl, pyrazolyl, pyridinyl, pyrazinyl, pyridazinyl and pyrimidinyl, and the like. Unless specified otherwise, the heteroaryl ring may be substituted at one or more ring positions with, for example, halogen, azide, alkyl, aralkyl, alkenyl, alkynyl, cycloalkyl, hydroxyl, alkoxyl, amino, nitro, sulfhydryl, imino, amido, carboxylic acid, C(O)alkyl, —CO₂alkyl, carbonyl, carboxyl, alkylthio, sulfonyl, sulfonamido, sulfonamide, ketone, aldehyde, ester, heterocyclyl, aryl or heteroaryl moieties, —CF₃, —CN, or the like. The term “heteroaryl” also includes polycyclic ring systems having two or more rings in which two or more carbons are common to two adjoining rings (the rings are “fused rings”) wherein at least one of the rings is heteroaromatic, e.g., the other cyclic rings may be cycloalkyls, cycloalkenyls, cycloalkynyls, and/or aryls. In certain embodiments, the heteroaryl ring is substituted at one or more ring positions with halogen, alkyl, hydroxyl, or alkoxyl. In certain other embodiments, the heteroaryl ring is not substituted, i.e., it is unsubstituted. In certain embodiments, the heteroaryl group is a 5- to 10-membered ring structure, alternatively a 5- to 6-membered ring structure, whose ring structure includes 1, 2, 3, or 4 heteroatoms, such as nitrogen, oxygen, and sulfur.

The terms “amine” and “amino” are art-recognized and refer to both unsubstituted and substituted amines, e.g., a moiety represented by the general formula —N(R¹⁰)(R¹¹), wherein R¹⁰ and R¹¹ each independently represent hydrogen, alkyl, cycloalkyl, heterocyclyl, alkenyl, aryl, aralkyl, or (CH₂)ₘ—R¹²; or R¹⁰ and R¹¹, taken together with the N atom to which they are attached complete a heterocycle having from 4 to 8 atoms in the ring structure; R¹² represents an aryl, a cycloalkyl, a cycloalkenyl, a heterocycle or a polycycle; and m is zero or an integer in the range of 1 to 8. In certain embodiments, R¹⁰ and R¹¹ each independently represent hydrogen, alkyl, alkenyl, or —(CH₂)ₘ—R¹².

The terms “alkoxyl” or “alkoxy” are art-recognized and refer to an alkyl group, as defined above, having an oxygen radical attached thereto. In some embodiments, alkoxyl is optionally substituted. Representative alkoxyl groups include methoxy, ethoxy, propyloxy, tert-butoxy and the like. An “ether” is two hydrocarbons covalently linked by an oxygen. Accordingly, the substituent of an alkyl that renders that alkyl an ether is or resembles an alkoxyl, such as may be represented by one of —O-alkyl, —O-alkenyl, O-alkynyl, —O—(CH₂)ₘ—R¹², where m and R¹² are described above. The term “haloalkoxyl” refers to an alkoxyl group that is substituted with at least one halogen. For example, —O—CH₂F, —O—CHF₂, —O—CF₃, and the like. In certain embodiments, the haloalkoxyl is an alkoxyl group that is substituted with at least one fluoro group. In certain embodiments, the haloalkoxyl is an alkoxyl group that is substituted with from 1-6, 1-5, 1-4, 2-4, or 3 fluoro groups.

The symbol “ [symbol] ” indicates a point of attachment.

The compounds of the disclosure may contain one or more chiral centers and/or double bonds and, therefore, exist as stereoisomers, such as geometric isomers, enantiomers or diastereomers. The term “stereoisomers” when used herein consist of all geometric isomers, enantiomers or diastereomers. These compounds may be designated by the symbols “R” or “S,” depending on the configuration of substituents around the stereogenic carbon atom. The present invention encompasses various stereoisomers of these compounds and mixtures thereof. Stereoisomers include enantiomers and diastereomers. Mixtures of enantiomers or diastereomers may be designated “(±)” in nomenclature, but the skilled artisan will recognize that a structure may denote a chiral center implicitly. It is understood that graphical depictions of chemical structures, e.g., generic chemical structures, encompass all stereoisomeric forms of the specified compounds, unless indicated otherwise.

Individual stereoisomers of compounds of the present invention can be prepared synthetically from commercially available starting materials that contain asymmetric or stereogenic centers, or by preparation of racemic mixtures followed by resolution methods well known to those of ordinary skill in the art. These methods of resolution are exemplified by (1) attachment of a mixture of enantiomers to a chiral auxiliary, separation of the resulting mixture of diastereomers by recrystallization or chromatography and liberation of the optically pure product from the auxiliary, (2) salt formation employing an optically active resolving agent, or (3) direct separation of the mixture of optical enantiomers on chiral chromatographic columns. Stereoisomeric mixtures can also be resolved into their component stereoisomers by well-known methods, such as chiral-phase gas chromatography, chiral-phase high performance liquid chromatography, crystallizing the compound as a chiral salt complex, or crystallizing the compound in a chiral solvent. Further, enantiomers can be separated using supercritical fluid chromatographic (SFC) techniques described in the literature. Still further, stereoisomers can be obtained from stereomerically-pure intermediates, reagents, and catalysts by well-known asymmetric synthetic methods.

Geometric isomers can also exist in the compounds of the present invention. The symbol “ [symbol] ” denotes a bond that may be a single, double or triple bond as described herein. The present invention encompasses the various geometric isomers and mixtures thereof resulting from the arrangement of substituents around a carbon-carbon double bond or arrangement of substituents around a carbocyclic ring. Substituents around a carbon-carbon double bond are designated as being in the “Z” or “E” configuration wherein the terms “Z” and “E” are used in accordance with IUPAC standards. Unless otherwise specified, structures depicting double bonds encompass both the “E” and “Z” isomers.

Substituents around a carbon-carbon double bond alternatively can be referred to as “cis” or “trans,” where “cis” represents substituents on the same side of the double bond and “trans” represents substituents on opposite sides of the double bond. The arrangement of substituents around a carbocyclic ring are designated as “cis” or “trans.” The term “cis” represents substituents on the same side of the plane of the ring and the term “trans” represents substituents on opposite sides of the plane of the ring. Mixtures of compounds wherein the substituents are disposed on both the same and opposite sides of plane of the ring are designated “cis/trans.”

The invention also embraces isotopically labeled compounds of the invention which are identical to those recited herein, except that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorus, fluorine and chlorine, such as ²H, ³H, ¹³C, ¹⁴C, ¹⁵N, ¹⁸O, ¹⁷O ³¹P, ³²P, ³⁵S, ¹⁸F, and ³⁶Cl, respectively.

Certain isotopically-labeled disclosed compounds (e.g., those labeled with 3H and 14C) are useful in compound and/or substrate tissue distribution assays. Tritiated (i.e., 3H) and carbon-14 (i.e., 14C) isotopes are particularly preferred for their ease of preparation and detectability. Further, substitution with heavier isotopes such as deuterium (i.e., 2H) may afford certain therapeutic advantages resulting from greater metabolic stability (e.g., increased in vivo half-life or reduced dosage requirements) and hence may be preferred in some circumstances. Isotopically labeled compounds of the invention can generally be prepared by following procedures analogous to those disclosed in, e.g., the Examples herein by substituting an isotopically labeled reagent for a non-isotopically labeled reagent.

As used herein, the terms “subject” and “patient” refer to organisms to be treated by the methods of the present invention. Such organisms are preferably mammals (e.g., murines, simians, equines, bovines, porcines, canines, felines, and the like), and more preferably humans.

As used herein, the term “pharmaceutical composition” refers to the combination of an active agent with a carrier, inert or active, making the composition especially suitable for diagnostic or therapeutic use in vivo or ex vivo.

As used herein, the term “pharmaceutically acceptable excipient” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see Remington's The Science and Practice of Pharmacy, 21st Edition, A. R. Gennaro; Lippincott, Williams & Wilkins, Baltimore, M D, 2006.

As is known to those of skill in the art, “salts” of the compounds of the present invention may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic, benzenesulfonic acid, and the like. Other acids, such as oxalic, while not in themselves pharmaceutically acceptable, may be employed in the preparation of salts useful as intermediates in obtaining the compounds of the invention and their pharmaceutically acceptable acid addition salts.

Examples of bases include, but are not limited to, alkali metal (e.g., sodium) hydroxides, alkaline earth metal (e.g., magnesium) hydroxides, ammonia, and compounds of formula NW₄⁺, wherein W is C₁₋₄ alkyl, and the like.

Examples of salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like. Other examples of salts include anions of the compounds of the present invention compounded with a suitable cation such as Na⁺, NH₄⁺, and NW₄⁺ (wherein W is a C₁₋₄ alkyl group), and the like.

Abbreviations as used herein include diisopropylethylamine (DIPEA); 4-dimethylaminopyridine (DMAP); tetrabutylammonium iodide (TBAI); 1-ethyl-3-(3-dimethylaminopropyl)carbodiimide (EDC); benzotriazol-1-yl-oxytripyrrolidinophosphonium hexafluorophosphate (PyBOP), 9-Fluorenylmethoxycarbonyl (Fmoc), tetrabutyldimethylsilyl chloride (TBDMSCl), hydrogen fluoride (HF), phenyl (Ph), bis(trimethylsilyl)amine (HMDS), dimethylformamide (DMF); methylene chloride (DCM); tetrahydrofuran (THF); high-performance liquid chromatography (HPLC); mass spectrometry (MS), evaporative light scattering detector (ELSD), electrospray (ES)); nuclear magnetic resonance spectroscopy (NMR).

As used herein, the term “effective amount” refers to the amount of a compound (e.g., a nucleic acid, e.g., an mRNA) sufficient to effect beneficial or desired results. An effective amount can be administered in one or more administrations, applications or dosages and is not intended to be limited to a particular formulation or administration route. The term effective amount can be considered to include therapeutically and/or prophylactically effective amounts of a compound.

The phrase “therapeutically effective amount” as used herein means that amount of a compound (e.g., a nucleic acid, e.g., an mRNA), material, or composition comprising a compound (e.g., a nucleic acid, e.g., an mRNA) which is effective for producing some desired therapeutic effect in at least a sub-population of cells in a mammal, for example, a human, or a subject (e.g., a human subject) at a reasonable benefit/risk ratio applicable to any medical treatment.

The phrase “prophylactically effective amount” as used herein means that amount of a compound (e.g., a nucleic acid, e.g., an mRNA), material, or composition comprising a compound (e.g., a nucleic acid, e.g., an mRNA) which is effective for producing some desired prophylactic effect in at least a sub-population of cells in a mammal, for example, a human, or a subject (e.g., a human subject) by reducing, minimizing or eliminating the risk of developing a condition or the reducing or minimizing severity of a condition at a reasonable benefit/risk ratio applicable to any medical treatment.

As used herein, the terms “treat,” “treating,” and “treatment” include any effect, e.g., lessening, reducing, modulating, ameliorating or eliminating, that results in the improvement of the condition, disease, disorder, and the like, or ameliorating a symptom thereof.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition or a method described herein can be combined in a variety of ways without departing from the spirit and scope of the present invention, whether explicit or implicit herein. For example, where reference is made to a particular compound, that compound can be used in various embodiments of compositions of the present invention and/or in methods of the present invention, unless otherwise understood from the context. In other words, within this application, embodiments have been described and depicted in a way that enables a clear and concise application to be written and drawn, but it is intended and will be appreciated that embodiments may be variously combined or separated without parting from the present teachings and invention(s). For example, it will be appreciated that all features described and depicted herein can be applicable to all aspects of the invention(s) described and depicted herein.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

Where the use of the term “about” is before a quantitative value, the present invention also includes the specific quantitative value itself, unless specifically stated otherwise. As used herein, the term “about” refers to a ±10% variation from the nominal value unless otherwise indicated or inferred.

As used herein, unless otherwise indicated, the term “antibody” means any antigen-binding molecule or molecular complex comprising at least one complementarity determining region (CDR) that specifically binds to or interacts with a particular antigen. It is understood the term encompasses an intact antibody (e.g., an intact monoclonal antibody), or a fragment thereof, such as an Fc fragment of an antibody (e.g., an Fc fragment of a monoclonal antibody), or an antigen-binding fragment of an antibody (e.g., an antigen-binding fragment of a monoclonal antibody), including an intact antibody, antigen-binding fragment, or Fc fragment that has been modified or engineered. Examples of antigen-binding fragments include Fab, Fab′, (Fab′)₂, Fv, single chain antibodies (e.g., scFv), minibodies, and diabodies. Examples of antibodies that have been modified or engineered include chimeric antibodies, humanized antibodies, and multispecific antibodies (e.g., bispecific antibodies). The term also encompasses an immunoglobulin single variable domain, such as a Nanobody (e.g., a V_{HH}).

As used here, an “antibody that binds to X” (i.e., X being a particular antigen), or “an anti-X antibody”, is an antibody that specifically recognizes the antigen X.

As used herein, a “buried interchain disulfide bond” or an “interchain buried disulfide bond” refers to a disulfide bond on a polypeptide which is not readily accessible to water soluble reducing agents, or is effectively “buried” in the hydrophobic regions of the polypeptide, such that it is unavailable to both reducing agents and for conjugation to other hydrophilic PEGs. Buried interchain disulfide bonds are further described in WO2017096361A1, which is incorporated by reference in its entirety.

As used herein, specificity of the targeted delivery by an LNP is defined by the ratio between % of a desired immune cell type that receives the delivered nucleic acid (e.g., on-target delivery), and % of an undesired immune cell type that is not meant to be the destination of the delivery, but receives the delivered nucleic acid (e.g., off-target delivery). For example, the specificity is higher when more desired immune cells receive the delivered nucleic acid, while less undesired immune cells receive the delivered nucleic acid. Specificity of the targeted delivery by an LNP can also be defined the ratio of amount of nucleic acid being delivered to the desired immune cells (e.g., on-target delivery) and amount of nucleic acid being delivered to the undesired immune cells (e.g., off-target delivery). Specificity of the delivery can be determined using any suitable method. As a non-limiting example, expression level of the nucleic acid in the desired immune cell type can be measured and compared to that of a different immune cell type that is not meant to be the destination of the delivery.

As used herein, in some embodiments, a reference LNP is an LNP that does not have the immune cell targeting group but is otherwise the same as the tested LNP. In some other embodiments, a reference LNP is an LNP that has a different ionizable cationic lipid but is otherwise the same as the tested LNP. In some embodiments, a reference LNP comprises D-Lin-MC3-DMA as the ionizable cationic lipid which is different from the ionizable cationic lipid in a tested LNP, but is otherwise the same as the tested LNP.

As used herein, a humanized antibody is an antibody which is wholly or partially of non-human origin and whose protein sequence has been modified to replace certain amino acids, for instance that occur at the corresponding position(s) in the framework regions of the VH and VL domains in a sequence of antibody from a human being, to increase its similarity to antibodies produced naturally in humans, in order to avoid or minimize an immune response in humans. For example, using techniques of genetic engineering, the variable domains of a non-human antibodies of interest may be combined with the constant domains of human antibodies. The constant domains of a humanized antibody are most of the time human CH and CL domains.

As used herein, the term “structural lipid” refers to sterols and also to lipids containing sterol moieties.

It should be understood that the order of steps or order for performing certain actions is immaterial so long as the present invention remain operable. Moreover, two or more steps or actions may be conducted simultaneously.

At various places in the present specification, substituents are disclosed in groups or in ranges. It is specifically intended that the description include each and every individual subcombination of the members of such groups and ranges. For example, the term “C₁₋₆ alkyl” is specifically intended to individually disclose C₁, C₂, C₃, C₄, C₅, C₆, C₁-C₆, C₁-C₅, C₁-C₄, C₁-C₃, C₁-C₂, C₂-C₆, C₂-C₅, C₂-C₄, C₂-C₃, C₃-C₆, C₃-C₅, C₃-C₄, C₄-C₆, C₄-C₅, and C₅-C₆ alkyl. By way of other examples, an integer in the range of 0 to 40 is specifically intended to individually disclose 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, and 40, and an integer in the range of 1 to 20 is specifically intended to individually disclose 1, 2, 3, 4, 5, 6, 7, 8, 9, 10, 11, 12, 13, 14, 15, 16, 17, 18, 19, and 20.

The use of any and all examples, or exemplary language herein, for example, “such as” or “including,” is intended merely to illustrate better the present invention and does not pose a limitation on the scope of the invention unless claimed. No language in the specification should be construed as indicating any non-claimed element as essential to the practice of the present invention.

Throughout the description, where compositions and kits are described as having, including, or comprising specific components, or where processes and methods are described as having, including, or comprising specific steps, it is contemplated that, additionally, there are compositions and kits of the present invention that consist essentially of, or consist of, the recited components, and that there are processes and methods according to the present invention that consist essentially of, or consist of, the recited processing steps.

As a general matter, compositions specifying a percentage are by weight unless otherwise specified. Further, if a variable is not accompanied by a definition, then the previous definition of the variable controls.

### Immunoglobulin Single Variable Domain

In some embodiments, the immune cell targeting group of the LNPs as described herein comprise an immunoglobulin single variable domain, such as an Nanobody.

The term “immunoglobulin single variable domain” (ISV), interchangeably used with “single variable domain,” defines immunoglobulin molecules wherein the antigen binding site is present on, and formed by, a single immunoglobulin domain. This sets immunoglobulin single variable domains apart from “conventional” immunoglobulins (e.g., monoclonal antibodies) or their fragments (such as Fab, Fab′, F(ab′)₂, scFv, di-scFv), wherein two immunoglobulin domains, in particular two variable domains, interact to form an antigen binding site. Typically, in conventional immunoglobulins, a heavy chain variable domain (V_{H}) and a light chain variable domain (V_{L}) interact to form an antigen binding site. In this case, the complementarity determining regions (CDRs) of both V_{H} and V_{L} will contribute to the antigen binding site, i.e. a total of 6 CDRs will be involved in antigen binding site formation. In view of the above definition, the antigen-binding domain of a conventional 4-chain antibody (such as an IgG, IgM, IgA, IgD or IgE molecule; known in the art) or of a Fab, a F(ab′)₂ fragment, an Fv fragment such as a disulfide linked Fv or a scFv fragment, or a diabody (all known in the art) derived from such conventional 4-chain antibody, would normally not be regarded as an immunoglobulin single variable domain, as, in these cases, binding to the respective epitope of an antigen would normally not occur by one (single) immunoglobulin domain but by a pair of (associating) immunoglobulin domains such as light and heavy chain variable domains, i.e., by a V_{H}-V_{L} pair of immunoglobulin domains, which jointly bind to an epitope of the respective antigen.

In contrast, immunoglobulin single variable domains are capable of specifically binding to an epitope of the antigen without pairing with an additional immunoglobulin variable domain. The binding site of an immunoglobulin single variable domain is formed by a single V_{H}, a single V_{HH} or single V_{L} domain. Hence, the antigen binding site of an immunoglobulin single variable domain is formed by no more than three CDRs.

As such, the single variable domain may be a light chain variable domain sequence (e.g., a V_{L}-sequence) or a suitable fragment thereof; or a heavy chain variable domain sequence (e.g., a V_{H}-sequence or V_{HH} sequence) or a suitable fragment thereof; as long as it is capable of forming a single antigen binding unit (i.e., a functional antigen binding unit that essentially consists of the single variable domain, such that the single antigen binding domain does not need to interact with another variable domain to form a functional antigen binding unit).

An immunoglobulin single variable domain (ISV) can for example be a heavy chain ISV, such as a V_{H}, V_{HH}, including a camelized V_{H} or humanized V_{HH}. In one embodiment, it is a V_{HH}, including a camelized V_{H} or humanized V_{HH}. Heavy chain ISVs can be derived from a conventional four-chain antibody or from a heavy chain antibody.

For example, the immunoglobulin single variable domain may be a (single) domain antibody (or an amino acid sequence that is suitable for use as a single domain antibody), a “dAb” or dAb (or an amino acid sequence that is suitable for use as a dAb) or a Nanobody® ISV (as defined herein and including but not limited to a V_{HH}); other single variable domains, or any suitable fragment of any one thereof.

In particular, the immunoglobulin single variable domain may be a Nanobody® ISV (such as a V_{HH}, including a humanized V_{HH} or camelized V_{H}) or a suitable fragment thereof. [Note: Nanobody® is a registered trademark of Ablynx N.V.].

“V_{HH} domains”, also known as V_{HH}s, V_{HH} antibody fragments, and V_{HH} antibodies, have originally been described as the antigen binding immunoglobulin variable domain of “heavy chain antibodies” (i.e., of “antibodies devoid of light chains”; Hamers-Casterman et al. 1993 (Nature 363: 446-448). The term “V_{HH} domain” has been chosen in order to distinguish these variable domains from the heavy chain variable domains that are present in conventional 4-chain antibodies (which are referred to herein as “V_{H} domains”) and from the light chain variable domains that are present in conventional 4-chain antibodies (which are referred to herein as “V_{L} domains”). For a further description of V_{HH}'s, reference is made to the review article by Muyldermans 2001 (Reviews in Molecular Biotechnology 74: 277-302).

For the term “dAb's” and “domain antibody”, reference is for example made to Ward et al. 1989 (Nature 341: 544), to Holt et al. 2003 (Trends Biotechnol. 21: 484); as well as to for example WO 2004/068820, WO 2006/030220, WO 2006/003388 and other published patent applications of Domantis Ltd. It should also be noted that, although less preferred in the context of the present invention because they are not of mammalian origin, single variable domains can be derived from certain species of shark (for example, the so-called “IgNAR domains”, see for example WO 2005/18629).

Typically, the generation of immunoglobulins involves the immunization of experimental animals, fusion of immunoglobulin producing cells to create hybridomas and screening for the desired specificities. Alternatively, immunoglobulins can be generated by screening of naïve, immune or synthetic libraries, e.g., by phage display.

The generation of immunoglobulin sequences, such as VHHs, has been described extensively in various publications, among which WO 1994/04678, Hamers-Casterman et al. 1993 (Nature 363: 446-448) and Muyldermans et al. 2001 (Reviews in Molecular Biotechnology 74: 277-302, 2001). In these methods, camelids are immunized with the target antigen in order to induce an immune response against said target antigen. The repertoire of VHHs obtained from said immunization is further screened for VHHs that bind the target antigen.

In these instances, the generation of antibodies requires purified antigen for immunization and/or screening. Antigens can be purified from natural sources, or in the course of recombinant production. Immunization and/or screening for immunoglobulin sequences can be performed using peptide fragments of such antigens.

Immunoglobulin sequences of different origin, comprising mouse, rat, rabbit, donkey, human and camelid immunoglobulin sequences can be used herein. Also, fully human, humanized or chimeric sequences can be used in the method described herein. For example, camelid immunoglobulin sequences and humanized camelid immunoglobulin sequences, or camelized domain antibodies, e.g., camelized dAb as described by Ward et al. 1989 (Nature 341: 544), WO 1994/04678, and Davis and Riechmann (1994, Febs Lett., 339:285-290; and 1996, Prot. Eng., 9:531-537) can be used herein. Moreover, the ISVs are fused forming a multivalent and/or multispecific construct (for multivalent and multispecific polypeptides containing one or more V_{HH} domains and their preparation, reference is also made to Conrath et al. 2001 (J. Biol. Chem., Vol. 276, 10. 7346-7350) as well as to for example WO 1996/34103 and WO 1999/23221).

A “humanized V_{HH}” comprises an amino acid sequence that corresponds to the amino acid sequence of a naturally occurring V_{HH} domain, but that has been “humanized”, i.e. by replacing one or more amino acid residues in the amino acid sequence of said naturally occurring V_{HH} sequence (and in particular in the framework sequences) by one or more of the amino acid residues that occur at the corresponding position(s) in a V_{H} domain from a conventional 4-chain antibody from a human being (e.g., indicated above). This can be performed in a manner known per se, which will be clear to the skilled person, for example on the basis of the prior art (e.g., WO 2008/020079). Again, it should be noted that such humanized V_{HH}S can be obtained in any suitable manner known per se and thus are not strictly limited to polypeptides that have been obtained using a polypeptide that comprises a naturally occurring VHH domain as a starting material.

A “camelized V_{H}” comprises an amino acid sequence that corresponds to the amino acid sequence of a naturally occurring V_{H} domain, but that has been “camelized”, i.e. by replacing one or more amino acid residues in the amino acid sequence of a naturally occurring V_{H} domain from a conventional 4-chain antibody by one or more of the amino acid residues that occur at the corresponding position(s) in a V_{HH} domain of a (camelid) heavy chain antibody. This can be performed in a manner known per se, which will be clear to the skilled person, for example on the basis of the description in the prior art (e.g., Davies and Riechman 1994, FEBS 339: 285; 1995, Biotechnol. 13: 475; 1996, Prot. Eng. 9: 531; and Riechman 1999, J. Immunol. Methods 231: 25). Such “camelizing” substitutions are inserted at amino acid positions that form and/or are present at the V_{H}-V_{L} interface, and/or at the so-called Camelidae hallmark residues, as defined herein (see for example WO 1994/04678 and Davies and Riechmann (1994 and 1996, supra). In one embodiment, the V_{H} sequence that is used as a starting material or starting point for generating or designing the camelized V_{H} is a V_{H} sequence from a mammal, such as the V_{H} sequence of a human being, such as a V_{H}3 sequence. However, it should be noted that such camelized V_{H} can be obtained in any suitable manner known per se and thus are not strictly limited to polypeptides that have been obtained using a polypeptide that comprises a naturally occurring V_{H} domain as a starting material.

The structure of an immunoglobulin single variable domain sequence can be considered to be comprised of four framework regions (“FRs”), which are referred to in the art and herein as “Framework region 1” (“FR1”); as “Framework region 2” (“FR2”); as “Framework region 3” (“FR3”); and as “Framework region 4” (“FR4”), respectively; which framework regions are interrupted by three complementary determining regions (“CDRs”), which are referred to in the art and herein as “Complementarity Determining Region 1” (“CDR1”); as “Complementarity Determining Region 2” (“CDR2”); and as “Complementarity Determining Region 3” (“CDR3”), respectively.

In such an immunoglobulin sequence, the framework sequences may be any suitable framework sequences, and examples of suitable framework sequences will be clear to the skilled person, for example on the basis the standard handbooks and the further disclosure and prior art mentioned herein.

The framework sequences are (a suitable combination of) immunoglobulin framework sequences or framework sequences that have been derived from immunoglobulin framework sequences (for example, by humanization or camelization). For example, the framework sequences may be framework sequences derived from a light chain variable domain (e.g., a V_{L}-sequence) and/or from a heavy chain variable domain (e.g., a V_{H}-sequence or V_{HH} sequence). In one particular aspect, the framework sequences are either framework sequences that have been derived from a V_{HH}-sequence (in which said framework sequences may optionally have been partially or fully humanized) or are conventional V_{H} sequences that have been camelized (as defined herein).

In particular, the framework sequences present in the ISV sequence described herein may contain one or more of hallmark residues (as defined herein), such that the ISV sequence is a Nanobody® ISV, such as, e.g., a V_{HH}, including a humanized V_{HH} or camelized V_{H}. Non-limiting examples of (suitable combinations of) such framework sequences will become clear from the further disclosure herein.

The total number of amino acid residues in a V_{H} domain and a V_{HH} domain will usually be in the range of from 110 to 120, often between 112 and 115. It should however be noted that smaller and longer sequences may also be suitable for the purposes described herein.

However, it should be noted that the ISVs described herein is not limited as to the origin of the ISV sequence (or of the nucleotide sequence used to express it), nor as to the way that the ISV sequence or nucleotide sequence is (or has been) generated or obtained. Thus, the ISV sequences may be naturally occurring sequences (from any suitable species) or synthetic or semi-synthetic sequences. In a specific but non-limiting aspect, the ISV sequence is a naturally occurring sequence (from any suitable species) or a synthetic or semi-synthetic sequence, including but not limited to “humanized” (as defined herein) immunoglobulin sequences (such as partially or fully humanized mouse or rabbit immunoglobulin sequences, and in particular partially or fully humanized V_{HH} sequences), “camelized” (as defined herein) immunoglobulin sequences (and in particular camelized V_{H} sequences), as well as ISVs that have been obtained by techniques such as affinity maturation (for example, starting from synthetic, random or naturally occurring immunoglobulin sequences), CDR grafting, veneering, combining fragments derived from different immunoglobulin sequences, PCR assembly using overlapping primers, and similar techniques for engineering immunoglobulin sequences well known to the skilled person; or any suitable combination of any of the foregoing.

Similarly, nucleotide sequences may be naturally occurring nucleotide sequences or synthetic or semi-synthetic sequences, and may for example be sequences that are isolated by PCR from a suitable naturally occurring template (e.g., DNA or RNA isolated from a cell), nucleotide sequences that have been isolated from a library (and in particular, an expression library), nucleotide sequences that have been prepared by introducing mutations into a naturally occurring nucleotide sequence (using any suitable technique known per se, such as mismatch PCR), nucleotide sequence that have been prepared by PCR using overlapping primers, or nucleotide sequences that have been prepared using techniques for DNA synthesis known per se.

Generally, Nanobody® ISVs (in particular V_{HH} sequences, including (partially) humanized V_{HH} sequences and camelized V_{H} sequences) can be characterized by the presence of one or more “Hallmark residues” (as described herein) in one or more of the framework sequences (again as further described herein). Thus, generally, a Nanobody® ISV can be defined as an immunoglobulin sequence with the (general) structure

FR1-CDR1-FR2-CDR2-FR3-CDR3-FR4

in which FR1 to FR4 refer to framework regions 1 to 4, respectively, and in which CDR1 to CDR3 refer to the complementarity determining regions 1 to 3, respectively, and in which one or more of the Hallmark residues are as further defined herein.

In particular, a Nanobody® ISV can be an immunoglobulin sequence with the (general) structure

FR1-CDR1-FR2-CDR2-FR3-CDR3-FR4

in which FR1 to FR4 refer to framework regions 1 to 4, respectively, and in which CDR1 to CDR3 refer to the complementarity determining regions 1 to 3, respectively, and in which the framework sequences are as further defined herein.

More in particular, a Nanobody® ISV can be an immunoglobulin sequence with the (general) structure

FR1-CDR1-FR2-CDR2-FR3-CDR3-FR4

in which FR1 to FR4 refer to framework regions 1 to 4, respectively, and in which CDR1 to CDR3 refer to the complementarity determining regions 1 to 3, respectively, and in which: one or more of the amino acid residues at positions 11, 37, 44, 45, 47, 83, 84, 103, 104 and 108 according to the Kabat numbering are chosen from the Hallmark residues mentioned in Table A below.

TABLE A
Hallmark Residues in Nanobody ® ISVs

| Position | Human V_{H}3 | Hallmark Residues |
|---|---|---|
| 11 | L, V; predominantly L | L, S, V, M, W, F, T, Q, E, A, R, G, K, Y, N, P, I; |
| | | preferably L |
| 37 | V, I, F; usually V | F⁽¹⁾, Y, V, L, A, H, S, I, W, C, N, G, D, T, P, |
| | | preferably F⁽¹⁾ or Y |
| 44⁽⁸⁾ | G | E⁽³⁾, Q⁽³⁾, G⁽²⁾, D, A, K, R, L, P, S, V, H, T, N, W, |
| | | M, I; |
| | | preferably G⁽²⁾, E⁽³⁾ or Q⁽³⁾; most preferably G⁽²⁾ or |
| | | Q⁽³⁾ |
| 45⁽⁸⁾ | L | L⁽²⁾, R⁽³⁾, P, H, F, G, Q, S, E, T, Y, C, I, D, V; |
| | | preferably L⁽²⁾ or R⁽³⁾ |
| 47⁽⁸⁾ | W, Y | F⁽¹⁾, L⁽¹⁾ or W⁽²⁾ G, I, S, A, V, M, R, Y, E, P, T, C, H, |
| | | K, Q, N, D; preferably W^{(2),} L⁽¹⁾ or F⁽¹⁾ |
| 83 | R or K; usually R | R, K⁽⁵⁾, T, E⁽⁵⁾, Q, N, S, I, V, G, M, L, A, D, Y, H; |
| | | preferably K or R; most preferably K |
| 84 | A, T, D; predominantly A | P⁽⁵⁾, S, H, L, A, V, I, T, F, D, R, Y, N, Q, G, E; |
| | | preferably P |
| 103 | W | W⁽⁴⁾, R⁽⁶⁾, G, S, K, A, M, Y, L, F, T, N, V, Q, P⁽⁶⁾, E, |
| | | C; preferably W |
| 104 | G | G, A, S, T, D, P, N, E, C, L; preferably G |
| 108 | L, M or T; predominantly | Q, L⁽⁷⁾, R, P, E, K, S, T, M, A, H; preferably Q or |
| | L | L⁽⁷⁾ |

Notes:
In particular, but not exclusively, in combination with KERE (SEQ ID NO: 103) or KQRE (SEQ ID NO: 104) at positions 43-46.
Usually as GLEW (SEQ ID NO: 105) at positions 44-47.
Usually as KERE (SEQ ID NO: 103) or KQRE (SEQ ID NO: 104) at positions 43-46, e.g., as KEREL (SEQ ID NO: 106), KEREF (SEQ ID NO: 107), KQREL (SEQ ID NO: 108), KQREF (SEQ ID NO: 109), KEREG (SEQ ID NO: 110), KQREW (SEQ ID NO: 111) or KQREG (SEQ ID NO: 112) at positions 43-47. Alternatively, also sequences such as TERE (SEQ ID NO: 113) (for example TEREL (SEQ ID NO: 114)), TQRE (SEQ ID NO: 115) (for example TQREL (SEQ ID NO: 116)), KECE (SEQ ID NO: 117) (for example KECEL (SEQ ID NO: 118) or KECER (SEQ ID NO: 119)), KQCE (SEQ ID NO: 120) (for example KQCEL (SEQ ID NO: 121)), RERE (SEQ ID NO: 122) (for example REREG (SEQ ID NO: 123)), RQRE (SEQ ID NO: 124) (for example RQREL (SEQ ID NO: 125), RQREF (SEQ ID NO: 126) or RQREW (SEQ ID NO: 127)), QERE (SEQ ID NO: 128) (for example QEREG (SEQ ID NO: 129)), QQRE (SEQ ID NO: 130), (for example QQREW (SEQ ID NO: 131), QQREL (SEQ ID NO: 132) or QQREF (SEQ ID NO: 133)), KGRE (SEQ ID NO: 134) (for example KGREG (SEQ ID NO: 135)), KDRE (SEQ ID NO: 136) (for example KDREV (SEQ ID NO: 137)) are possible. Some other possible, but less preferred sequences include for example DECKL (SEQ ID NO: 138) and NVCEL (SEQ ID NO: 139).
With both GLEW (SEQ ID NO: 105) at positions 44-47 and KERE (SEQ ID NO: 103) or KQRE (SEQ ID NO: 104) at positions 43-46.
Often as KP or EP at positions 83-84 of naturally occurring V_{HH} domains.
In particular, but not exclusively, in combination with GLEW (SEQ ID NO: 105) at positions 44-47.
With the proviso that when positions 44-47 are GLEW (SEQ ID NO: 105), position 108 is always Q in (non-humanized) V_{HH} sequences that also contain a W at 103.
The GLEW group also contains GLEW-like sequences at positions 44-47, such as for example GVEW (SEQ ID NO: 140), EPEW (SEQ ID NO: 141), GLER (SEQ ID NO: 142), DQEW (SEQ ID NO: 143), DLEW (SEQ ID NO: 144), GIEW (SEQ ID NO: 145), ELEW (SEQ ID NO: 146), GPEW (SEQ ID NO: 147), EWLP (SEQ ID NO: 148), GPER (SEQ ID NO: 149), GLER (SEQ ID NO: 142) and ELEW (SEQ ID NO: 146).

In one embodiment, the immunoglobulin single variable domain has certain amino acid substitutions in the framework regions effective in preventing or reducing binding of so-called “pre-existing antibodies” to the polypeptides. ISVs in which (i) the amino acid residue at position 112 is one of K or Q; and/or (ii) the amino acid residue at position 89 is T; and/or (iii) the amino acid residue at position 89 is L and the amino acid residue at position 110 is one of K or Q; and (iv) in each of cases (i) to (iii), the amino acid at position 11 is preferably V have been described in WO2015/173325.

### Polypeptides

The immunoglobulin single variable domains may form part of a protein or polypeptide, which may comprise or essentially consist of one or more (at least one) immunoglobulin single variable domains and which may optionally further comprise one or more further amino acid sequences (all optionally linked via one or more suitable linkers). The term “immunoglobulin single variable domain” may also encompass such polypeptides. The one or more immunoglobulin single variable domains may be used as a binding unit in such a protein or polypeptide, which may optionally contain one or more further amino acids that can serve as a binding unit, so as to provide a monovalent, multivalent or multispecific polypeptide of the invention, respectively (for multivalent and multispecific polypeptides containing one or more VHH domains and their preparation, reference is also made to Conrath et al. 2001 (J. Biol. Chem. 276: 7346), as well as to for example WO 1996/34103, WO 1999/23221 and WO 2010/115998).

The polypeptides may comprise or essentially consist of one immunoglobulin single variable domain, as outlined above. Such polypeptides are also referred to herein as monovalent polypeptides.

The term “multivalent” indicates the presence of multiple ISVs in a polypeptide. In one embodiment, the polypeptide is “bivalent”, i.e., comprises or consists of two ISVs. In one embodiment, the polypeptide is “trivalent”, i.e., comprises or consists of three ISVs. In another embodiment, the polypeptide is “tetravalent”, i.e. comprises or consists of four ISVDs. The polypeptide can thus be “bivalent”, “trivalent”, “tetravalent”, “pentavalent”, “hexavalent”, “heptavalent”, “octavalent”, “nonavalent”, etc., i.e., the polypeptide comprises or consists of two, three, four, five, six, seven, eight, nine, etc., ISVs, respectively. In one embodiment the multivalent ISV polypeptide is trivalent. In another embodiment the multivalent ISV polypeptide is tetravalent. In still another embodiment, the multivalent ISV polypeptide is pentavalent.

In one embodiment, the multivalent ISV polypeptide can also be multispecific. The term “multispecific” refers to binding to multiple different target molecules (also referred to as antigens). The multivalent ISV polypeptide can thus be “bispecific”, “trispecific”, “tetraspecific”, etc., i.e., can bind to two, three, four, etc., different target molecules, respectively.

For example, the polypeptide may be bispecific-trivalent, such as a polypeptide comprising or consisting of three ISVs, wherein two ISVs bind to a first target and one ISV binds to a second target different from the first target. In another example, the polypeptide may be trispecific-tetravalent, such as a polypeptide comprising or consisting of four ISVs, wherein one ISV binds to a first target, two ISVs bind to a second target different from the first target and one ISV binds to a third target different from the first and the second target. In still another example, the polypeptide may be trispecific-pentavalent, such as a polypeptide comprising or consisting of five ISVs, wherein two ISVs bind to a first target, two ISVs bind to a second target different from the first target and one ISV binds to a third target different from the first and the second target.

In one embodiment, the multivalent ISV polypeptide can also be multiparatopic. The term “multiparatopic” refers to binding to multiple different epitopes on the same target molecules (also referred to as antigens). The multivalent ISV polypeptide can thus be “biparatopic”, “triparatopic”, etc., i.e., can bind to two, three, etc., different epitopes on the same target molecules, respectively.

In another aspect, the polypeptide of the invention that comprises or essentially consists of one or more immunoglobulin single variable domains (or suitable fragments thereof), may further comprise one or more other groups, residues, moieties or binding units. Such further groups, residues, moieties, binding units or amino acid sequences may or may not provide further functionality to the immunoglobulin single variable domain (and/or to the polypeptide in which it is present) and may or may not modify the properties of the immunoglobulin single variable domain.

For example, such further groups, residues, moieties or binding units may be one or more additional amino acids, such that the compound, construct or polypeptide is a (fusion) protein or (fusion) polypeptide. In a preferred but non-limiting aspect, said one or more other groups, residues, moieties or binding units are immunoglobulins. Even more preferably, said one or more other groups, residues, moieties or binding units are chosen from the group consisting of domain antibodies, amino acids that are suitable for use as a domain antibody, single domain antibodies, amino acids that are suitable for use as a single domain antibody, “dAb”s, amino acids that are suitable for use as a dAb, or Nanobodies.

Alternatively, such groups, residues, moieties or binding units may for example be chemical groups, residues, moieties, which may or may not by themselves be biologically and/or pharmacologically active. For example, and without limitation, such groups may be linked to the one or more immunoglobulin single variable domain so as to provide a “derivative” of the immunoglobulin single variable domain.

In another embodiment, said further residues may be effective in preventing or reducing binding of so-called “pre-existing antibodies” to the polypeptides. For this purpose, the polypeptides and constructs may contain a C-terminal extension (X)n (SEQ ID NO: 150) (in which n is 1 to 10, preferably 1 to 5, such as 1, 2, 3, 4 or 5 (and preferably 1 or 2, such as 1); and each X is an (preferably naturally occurring) amino acid residue that is independently chosen, and preferably independently chosen from the group consisting of alanine (A), glycine (G), valine (V), leucine (L) or isoleucine (I), for which reference is made to WO 2012/175741. Accordingly, the polypeptide may further comprise a C-terminal extension (X)n (SEQ ID NO: 151), in which n is 1 to 5, such as 1, 2, 3, 4 or 5, and in which X is a naturally occurring amino acid, preferably no cysteine.

In the polypeptides described above, the one or more immunoglobulin single variable domains and the one or more groups, residues, moieties or binding units may be linked directly to each other and/or via one or more suitable linkers or spacers. For example, when the one or more groups, residues, moieties or binding units are amino acids, the linkers may also be an amino acid, so that the resulting polypeptide is a fusion protein or fusion polypeptide.

As used herein, the term “linker” denotes a peptide that fuses together two or more ISVs into a single molecule. The use of linkers to connect two or more (poly)peptides is well known in the art. Further exemplary peptidic linkers are shown in Table B. One often used class of peptidic linker are known as the “Gly-Ser” or “GS” linkers. These are linkers that essentially consist of glycine (G) and serine (S) residues, and usually comprise one or more repeats of a peptide motif such as the GGGGS (SEQ ID NO:154) motif (for example, having the formula (Gly-Gly-Gly-Gly-Ser)n (SEQ ID NO: 152) in which n may be 1, 2, 3, 4, 5, 6, 7 or more). Some often-used examples of such GS linkers are 9GS linkers (GGGGSGGGS, SEQ ID NO: 157), 15GS linkers (n=3) and 35GS linkers (n=7). Reference is for example made to Chen et al. 2013 (Adv. Drug Deliv. Rev. 65(10): 1357-1369) and Klein et al. 2014 (Protein Eng. Des. Sel. 27 (10): 325-330).

TABLE B
Linker sequences
(“ID” refers to the SEQ ID NO as used herein)

| Name | ID | Amino acid sequence |
|---|---|---|
| 3A linker | 153 | AAA |
| 5GS linker | 154 | GGGGS |
| 7GS linker | 155 | SGGSGGS |
| 8GS linker | 156 | GGGGSGGS |
| 9GS linker | 157 | GGGGSGGGS |
| 10GS linker | 158 | GGGGSGGGGS |
| 15GS linker | 159 | GGGGSGGGGSGGGGS |
| 18GS linker | 160 | GGGGSGGGGSGGGGSGGS |
| 20GS linker | 161 | GGGGSGGGGSGGGGSGGGGS |
| 25GS linker | 162 | GGGGSGGGGSGGGGSGGGGSGGGGS |
| 30GS linker | 163 | GGGGSGGGGSGGGGSGGGGSGGGGSGGGGS |
| 35GS linker | 164 | GGGGSGGGGSGGGGSGGGGSGGGGSGGGGS |
| | | GGGGS |
| 40GS linker | 165 | GGGGSGGGGSGGGGSGGGGSGGGGSGGGGS |
| | | GGGGSGGGGS |
| G1 hinge | 166 | EPKSCDKTHTCPPCP |
| 9GS-G1 hinge | 167 | GGGGSGGGSEPKSCDKTHTCPPCP |
| Llama upper long | 168 | EPKTPKPQPAAA |
| hinge region | | |
| G3 hinge | 169 | ELKTPLGDTTHTCPRCPEPKSCDTPPPCPR |
| | | CPEPKSCDTPPPCPRCPEPKSCDTPPPCPR |
| | | CP |

In one aspect, the disclosure also relates to such amino acid sequences and/or Nanobodies that can bind to and/or are directed against CD8 and that comprise CDR sequences that are generally as further defined herein, to suitable fragments thereof, as well as to polypeptides that comprise or essentially consist of one or more of such Nanobodies and/or suitable fragments. In some aspect, the disclosure relates to Nanobodies with SEQ ID NO: 77. In particular, the disclosure in some specific aspects provides:
I) amino acid sequences that are directed against CD8 and that have at least 80%, preferably at least 85%, such as 90% or 95% or more sequence identity with SEQ ID NO: 77;
II) amino acid sequences that cross-block the binding of the amino acid sequence of SEQ ID NO: 77 to CD8 and/or that compete with at least the amino acid sequence of SEQ ID NO: 77 for binding to CD8;

Such amino acid sequences may be as further described herein (and may for example be Nanobodies); as well as polypeptides of the disclosure that comprise one or more of such amino acid sequences (which may be as further described herein), and particularly bispecific (or multispecific) polypeptides as described herein, and nucleic acid sequences that encode such amino acid sequences and polypeptides. Such amino acid sequences and polypeptides do not include any naturally occurring ligands.

In some embodiments, the CD8 is derived from a mammalian animal, such as a human being. In one specific, but non-limiting aspect, the disclosure relates to an amino acid sequence directed against CD8, that comprises:
a) the amino acid sequence of SEQ ID NO: 77;
b) amino acid sequences that have at least 80% amino acid identity with a SEQ ID NO: 77, or
c) amino acid sequences that have 3, 2, or 1 amino acid difference with SEQ ID NO: 77;
or any suitable combination thereof.

In some embodiments, disclosed is a Nanobody against CD8, which consist of 4 framework regions (FR1 to FR4 respectively) and 3 complementarity determining regions (CDR1 to CDR3 respectively). In some embodiments, in such a Nanobody:
(I) CDR1 comprises or essentially consists of an amino acid sequence of GSTFSDYG (SEQ ID NO: 100),
or amino acid sequences that have at least 80%, at least 90%, at least 95%, at least 99% or more sequence identity with GSTFSDYG (SEQ ID NO: 100), in which (1) any amino acid substitution is a conservative amino acid substitution; and/or (2) said amino acid sequence only contains amino acids substitutions, and no amino acid deletions or insertions, compared to GSTFSDYG (SEQ ID NO: 100);
and/or from the group consisting of amino acids sequences that have 2 or only 1 amino acid difference(s) with GSTFSDYG (SEQ ID NO: 100), in which
any amino acid substitution is a conservative amino acid substitution; and/or
said amino acid sequence only contains amino acid substitutions, and no amino acid deletions or insertions, compared to GSTFSDYG (SEQ ID NO: 100).
(II) CDR2 comprises or essentially consists of an amino acid sequence of IDWNGEHT (SEQ ID NO: 101),
or amino acid sequences that have at least 80%, at least 90%, at least 95%, at least 99% or more sequence identity with IDWNGEHT (SEQ ID NO: 101), in which (1) any amino acid substitution is a conservative amino acid substitution; and/or (2) said amino acid sequence only contains amino acids substitutions, and no amino acid deletions or insertions, compared to IDWNGEHT (SEQ ID NO: 101);
and/or from the group consisting of amino acids sequences that have 2 or only 1 amino acid difference(s) with IDWNGEHT (SEQ ID NO: 101), in which
any amino acid substitution is a conservative amino acid substitution; and/or
said amino acid sequence only contains amino acid substitutions, and no amino acid deletions or insertions, compared to IDWNGEHT (SEQ ID NO: 101).
(III) CDR3 comprises or essentially consists of an amino acid sequence of AADALPYTVRKYNY (SEQ ID NO: 102),
or amino acid sequences that have at least 80%, at least 90%, at least 95%, at least 99% or more sequence identity with AADALPYTVRKYNY (SEQ ID NO: 102), in which (1) any amino acid substitution is a conservative amino acid substitution; and/or (2) said amino acid sequence only contains amino acids substitutions, and no amino acid deletions or insertions, compared to AADALPYTVRKYNY (SEQ ID NO: 102);
and/or from the group consisting of amino acids sequences that have 2 or only 1 amino acid difference(s) with AADALPYTVRKYNY (SEQ ID NO: 102), in which
any amino acid substitution is a conservative amino acid substitution; and/or
said amino acid sequence only contains amino acid substitutions, and no amino acid deletions or insertions, compared to AADALPYTVRKYNY (SEQ ID NO: 102).
CD8 Nanobodies as disclosed herein may comprise one, two or all three of the CDRs explicitly listed above. In some embodiments, the CD8 Nanobody comprises:
CDR1: GSTFSDYG (SEQ ID NO: 100), based on IMGT designation;
CDR2: IDWNGEHT (SEQ ID NO: 101), based on IMGT designation; and
CDR3: AADALPYTVRKYNY (SEQ ID NO: 102), based on IMGT designation.

In the Nanobodies of the disclosure that comprise the combinations of CDRs mentioned above, each CDR can be replaced by a CDR chosen from the group consisting of amino acid sequences that have at least 80%, preferably at least 90%, more preferably at least 95%, even more preferably at least 99% sequence identity with the mentioned CDRs; in which:
(1) any amino acid substitution is preferably a conservative amino acid substitution; and/or
(2) said amino acid sequence preferably only contains amino acid substitutions, and no amino acid deletions or insertions, compared to the above amino acid sequence(s);
and/or chosen from the group consisting of amino acid sequences that have 3, 2 or only 1 (as indicated in the preceding paragraph) “amino acid difference(s)” with the mentioned CDR(s) one of the above amino acid sequences, in which:
(1) any amino acid substitution is preferably a conservative amino acid substitution; and/or
(2) said amino acid sequence preferably only contains amino acid substitutions, and no amino acid deletions or insertions, compared to the above amino acid sequence(s).

In one embodiment, the CD8 Nanobody is BDSn:

Anti-CD8 BDSn Nb sequence (CDR1, CDR2, CDR3 underlined based on IMGT designation):

(SEQ ID NO: 77)
EVQLVESGGGLVQAGGSLRLSCAASGSTFSDYGVGWFRQAPGKGREFVA
DIDWNGEHTSYADSVKGRFATSRDNAKNTAYLQMNSLKPEDTAVYYCAA
DALPYTVRKYNYWGQGTQVTVSSGGCGGHHHHHH

In some embodiments, a CD8 Nanobody of the present disclosure binds to CD8 with an dissociation constant (KD) of 10⁻⁵ to 10⁻¹² moles/liter (M) or less, and preferably 10⁻⁷ to 10-12 moles/liter (M) or less and more preferably 10⁻⁸ to 10⁻¹² moles/liter (M), and/or with an association constant (KA) of at least 107 M−1, preferably at least 10⁸ M⁻¹, more preferably at least 10⁹ M⁻¹, such as at least 10¹² M⁻¹; and in particular with a KD less than 500 nM, preferably less than 200 nM, more preferably less than 10 nM, such as less than 500 μM. The KD and KA values of the Nanobody of the disclosure against vWF can be determined in a manner known per se, for example using the assay described herein. More generally, the Nanobodies described herein preferably have a dissociation constant with respect to vWF that is as described in this paragraph.

Generally, it should be noted that the term Nanobody as used herein in its broadest sense is not limited to a specific biological source or to a specific method of preparation. For example, as will be discussed in more detail below, the Nanobodies can be obtained (1) by isolating the VHH domain of a naturally occurring heavy chain antibody; (2) by expression of a nucleotide sequence encoding a naturally occurring VHH domain; (3) by “humanization” (as described below) of a naturally occurring VHH domain or by expression of a nucleic acid encoding a such humanized VHH domain; (4) by “camelization” (as described below) of a naturally occurring VH domain from any animal species, in particular a species of mammal, such as from a human being, or by expression of a nucleic acid encoding such a camelized VH domain; (5) by “camelization” of a “domain antibody” or “Dab” as described by Ward et al (supra), or by expression of a nucleic acid encoding such a camelized VH domain; (6) using synthetic or semi-synthetic techniques for preparing proteins, polypeptides or other amino acid sequences; (7) by preparing a nucleic acid encoding a Nanobody using techniques for nucleic acid synthesis, followed by expression of the nucleic acid thus obtained; and/or (8) by any combination of the foregoing. Suitable methods and techniques for performing the foregoing will be clear to the skilled person based on the disclosure herein and for example include the methods and techniques described in more detail hereinbelow.

In some embodiments, the CD8 Nanobodies of the present disclosure do not have an amino acid sequence that is exactly the same as (i.e. as a degree of sequence identity of 100% with) the amino acid sequence of a naturally occurring VH domain, such as the amino acid sequence of a naturally occurring VH domain from a mammal, and in particular from a human being.

One class of CD8 Nanobodies of the disclosure comprises Nanobodies with an amino acid sequence that corresponds to the amino acid sequence of a naturally occurring VHH domain, but that has been “humanized”, i.e. by replacing one or more amino acid residues in the amino acid sequence of said naturally occurring VHH sequence by one or more of the amino acid residues that occur at the corresponding position(s) in a VH domain from a conventional 4-chain antibody from a human being (e.g., indicated above). It should be noted that such humanized CD8 Nanobodies of the present disclosure can be obtained in any suitable manner known per se (i.e. as indicated under points (1)-(8) above) and thus are not strictly limited to polypeptides that have been obtained using a polypeptide that comprises a naturally occurring VHH domain as a starting material.

Another class of CD8 Nanobodies of the present disclosure comprises Nanobodies with an amino acid sequence that corresponds to the amino acid sequence of a naturally occurring VH domain that has been “camelized”, i.e. by replacing one or more amino acid residues in the amino acid sequence of a naturally occurring VH domain from a conventional 4-chain antibody by one or more of the amino acid residues that occur at the corresponding position(s) in a VHH domain of a heavy chain antibody. This can be performed in a manner known per se, which will be clear to the skilled person, for example on the basis of the further description below. Reference is also made to WO 94/04678. Such camelization may preferentially occur at amino acid positions which are present at the VH-VL interface and at the so-called Camelidae hallmark residues (see for example also WO 94/04678), as also mentioned below. In some embodiments, the VH domain or sequence that is used as a starting material or starting point for generating or designing the camelized Nanobody is a VH sequence from a mammal, e.g., VH sequence of a human being. It should be noted that such camelized Nanobodies of the present disclosure can be obtained in any suitable manner known per se and thus are not strictly limited to polypeptides that have been obtained using a polypeptide that comprises a naturally occurring VH domain as a starting material.

For example, both “humanization” and “camelization” can be performed by providing a nucleotide sequence that encodes such a naturally occurring VHH domain or VH domain, respectively, and then changing, in a manner known per se, one or more codons in said nucleotide sequence such that the new nucleotide sequence encodes a humanized or camelized Nanobody of the present disclosure, respectively, and then expressing the nucleotide sequence thus obtained in a manner known per se so as to provide the desired Nanobody. Alternatively, based on the amino acid sequence of a naturally occurring VHH domain or VH domain, respectively, the amino acid sequence of the desired humanized or camelized Nanobody of the present disclosure, respectively, can be designed and then synthesized de novo using techniques for peptide synthesis known per se. Also, based on the amino acid sequence or nucleotide sequence of a naturally occurring VHH domain or VH domain, respectively, a nucleotide sequence encoding the desired humanized or camelized Nanobody can be designed and then synthesized de novo using techniques for nucleic acid synthesis known per se, after which the nucleotide sequence thus obtained can be expressed in a manner known per se so as to provide the desired Nanobody.

Other suitable ways and techniques for obtaining Nanobodies and/or nucleotide sequences and/or nucleic acids encoding the same, starting from (the amino acid sequence of) naturally occurring VH domains or preferably VHH domains and/or from nucleotide sequences and/or nucleic acid sequences encoding the same will be clear from the skilled person, and may for example comprising combining one or more amino acid sequences and/or nucleotide sequences from naturally occurring VH domains (such as one or more FR's and/or CDR's) with one or more one or more amino acid sequences and/or nucleotide sequences from naturally occurring VHH domains (such an one or more FR's or CDR's), in a suitable manner so as to provide (a nucleotide sequence or nucleic acid encoding) a Nanobody. Also provided are compounds and constructs, and in particular proteins and polypeptides that comprise or essentially consists of at least one such amino acid sequence and/or Nanobody of the disclosure (or suitable fragments thereof), and optionally further comprises one or more other groups, residues, moieties or binding units. In some embodiments, such further groups, residues, moieties, binding units or amino acid sequences may or may not provide further functionality to the amino acid sequence and/or Nanobody (and/or to the compound or construct in which it is present) and may or may not modify the properties of the amino acid sequence and/or Nanobody.

The disclosure also encompasses any polypeptide of the present disclosure that has been glycosylated at one or more amino acid positions, usually depending on the hot used to express the polypeptide. a polypeptide can comprise an amino acid sequence of a CD8 Nanobody of the present disclosure, which is fused at its amino terminal end, at its carboxy terminal end, or both at its amino terminal end and at its carboxy terminal end with at least one further amino acid sequence. Such further amino acid sequence may comprise at least one further Nanobody, so as to provide a polypeptide that comprises at least two, such as three, four or five, Nanobodies, in which said Nanobodies may optionally be linked via one or more linker sequences (as defined herein). Polypeptides of comprising CD8 Nanobody of the present disclosure and one or more another Nanobodies are multivalent polypeptides. In a multivalent polypeptide, the two or more Nanobodies may be the same or different. For example, the two or more Nanobodies in a multivalent polypeptide:
may be directed against the same antigen, i.e. against the same parts or epitopes of said antigen or against two or more different parts or epitopes of said antigen; and/or:
may be directed against the different antigens;
or a combination thereof.
   Thus, a bivalent polypeptide, for example:
may comprise two identical Nanobodies;
may comprise a first Nanobody directed against a first part or epitope of an antigen and a second Nanobody directed against the same part or epitope of said antigen or against another part or epitope of said antigen;
   or may comprise a first Nanobody directed against a first antigen and a second Nanobody directed against a second antigen different from said first antigen;
   whereas a trivalent Polypeptide of the Invention for example:
may comprise three identical or different Nanobodies directed against the same or different parts or epitopes of the same antigen;
may comprise two identical or different Nanobodies directed against the same or different parts or epitopes on a first antigen and a third Nanobody directed against a second antigen different from said first antigen; or
may comprise a first Nanobody directed against a first antigen, a second Nanobody directed against a second antigen different from said first antigen, and a third Nanobody directed against a third antigen different from said first and second antigen.

The CD8 Nanobodies and polypeptides as disclosed herein can also be introduced and expressed in one or more cells, tissues or organs of a multicellular organism, for example for prophylactic and/or therapeutic purposes (e.g., as a gene therapy). For this purpose, the nucleotide sequences encoding the CD8 Nanobodies or polypeptides as disclosed herein can be introduced into the cells or tissues in any suitable way, for example as such (e.g., using liposomes) or after they have been inserted into a suitable gene therapy vector (for example derived from retroviruses such as adenovirus, or parvoviruses such as adeno-associated virus). As will also be clear to the skilled person, such gene therapy may be performed in vivo and/or in situ in the body of a patent by administering a nucleic acid of the invention or a suitable gene therapy vector encoding the same to the patient or to specific cells or a specific tissue or organ of the patient; or suitable cells (often taken from the body of the patient to be treated, such as explanted lymphocytes, bone marrow aspirates or tissue biopsies) may be treated in vitro with a nucleotide sequence of the invention and then be suitably (re-)introduced into the body of the patient. All this can be performed using gene therapy vectors, techniques and delivery systems which are well known to the skilled person, for Culver, K. W., “Gene Therapy”, 1994, p. xii, Mary Ann Liebert, Inc., Publishers, New York, N.Y.). Giordano, Nature F Medicine 2 (1996), 534-539; Schaper, Circ. Res. 79 (1996), 911-919; Anderson, Science 256 (1992), 808-813; Verma, Nature 389 (1994), 239; Isner, Lancet 348 (1996), 370-374; Muhlhauser, Circ. Res. 77 (1995), 1077-1086; Onodera, Blood 91; (1998), 30-36; Verma, Gene Ther. 5 (1998), 692-699; Nabel, Ann. N.Y. Acad. Sci.: 811 (1997), 289-292; Verzeletti, Hum. Gene Ther. 9 (1998), 2243-51; Wang, Nature Medicine 2 (1996), 714-716; WO 94/29469; WO 97/00957, U.S. Pat. Nos. 5,580,859; 1 5,589,5466; or Schaper, Current Opinion in Biotechnology 7 (1996), 635-640. For example, in situ expression of ScFv fragments (Afanasieva et al., Gene Ther., 10, 1850-1859 (2003)) and of diabodies (Blanco et al., J. Immunol, 171, 1070-1077 (2003)) has been described in the art.

Accordingly, nucleic acid sequences encoding the CD8 Nanobodies as described herein, and expression construct and host cells comprising the nucleic acid sequence are also provided.

Also disclosed are methods of using CD8 Nanobodies and polypeptides of the present disclosure.

In some embodiments, a polypeptide comprising a CD8 Nanobody can be used in the lipid nanoparticles of the present disclosure for delivering a nucleic acid into an immune cell, as described herein. In some embodiments, CD8 Nanobodies and polypeptides of the present disclosure can be used to treat a condition or a disease in a subject in need thereof. In some embodiments, such conditions or diseases include, but are not limited to, cancer, infections, immune disorders, autoimmune diseases.

In some embodiments, a polypeptide comprising a CD8 Nanobody can be used in an imaging agent. In some embodiments, the imaging agent allows for the detection of human CD8 which is a specific biomarker found on the surface of a subset of T-cell for diagnostic imaging of the immune system. Imaging of CD8 allows for the in vivo detection of T-cell localization. Changes in T-cell localization can reflect the progression of an immune response and can occur over time as a result of various therapeutic treatments or even disease states. In some embodiments, it is used for imaging T-cell localization for immunotherapy.

In addition, CD8 plays a role in activating downstream signaling pathways that are important for the activation of cytolytic T cells that function to clear viral pathogens and provide immunity to tumors. CD8 positive T cells can recognize short peptides presented within the MHCI protein of antigen presenting cells. In some embodiments, a polypeptide comprising a CD8 Nanobody can potentiate signaling through the T cell receptor and enhance the ability of a subject to clear viral pathogens and respond to tumor antigens. Thus, in some embodiments, the antigen binding constructs provided herein can be agonists and can activate the CD8 target.

### II. Ionizable Cationic Lipids

Provided herein are ionizable cationic lipids that can be used to produce lipid nanoparticle compositions to facilitate the delivery of a payload (e.g., a nucleic acid, such as a DNA or RNA, such as an mRNA) disposed therein to cells, e.g., mammalian cells, e.g., immune cells. The ionizable cationic lipids have been designed to enable intracellular delivery of a nucleic acid, e.g., mRNA, to the cytosolic compartment of a target cell type and rapidly degrade into non-toxic components. The complex functionalities of the ionizable cationic lipids are facilitated by the interplay between the chemistry and geometry of the ionizable lipid head group, the hydrophobic “acyl-tail” groups and the linkers connecting the head group and the acyl tail groups. Typically, the pKₐ of the ionizable amine head group is designed to be in the range of 6-8, such as between 6.2-7.4, or between 6.7-7.2, such that it remains strongly cationic under acidic formulation conditions (e.g., pH 4-pH 5.5), neutral or slightly anionic in physiological pH (7.4) and cationic in the early and late endosomal compartments (e.g., pH 5.5-pH 7). The acyl-tail groups play a key role in fusion of the lipid nanoparticle with endosomal membranes and membrane destabilization through structural perturbation. The three-dimensional structure of the acyl-tail (determined by its length, and degree and site of unsaturation) along with the relative sizes of the head group and tail group are thought to play a role in promoting membrane fusion, and hence lipid nanoparticle endosomal escape (a key requirement for cytosolic delivery of a nucleic acid payload). The linker connecting the head group and acyl tail groups is designed to degrade by physiologically prevalent enzymes (e.g., esterases, or proteases) or by acid catalyzed hydrolysis.

In one aspect, the present invention provides a compound represented by Formula (I):

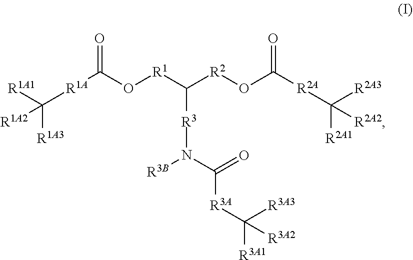

or a salt thereof, wherein:
R¹, R², and R³ are each independently a bond or C₁₋₃ alkylene;
R^{1A}, R^{2A}, and R^{3A} are each independently a bond or C₁₋₁₀ alkylene;
R^{1A1}, R^{1A2}, R^{1A3}, R^{2A1}, R^{2A2}, R^{2A3}, R^{3A1}, R^{3A2}, and R^{3A3} are each independently H, C₁₋₂₀ alkyl,
C₁₋₂₀ alkenyl, —(CH₂)₀₋₁₀C(O)OR^{a1}, or —(CH₂)₀₋₁₀OC(O)R^{a2};
R^{a1} and R^{a2} are each independently C₁₋₂₀ alkyl or C₁₋₂₀ alkenyl;
R^{3B} is

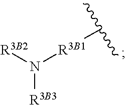

R^{3B1} is C₁₋₆ alkylene; and
R^{3B2} and R^{3B3} are each independently H or C₁₋₆ alkyl.

In one aspect, the present invention provides a compound represented by Formula (I-A):

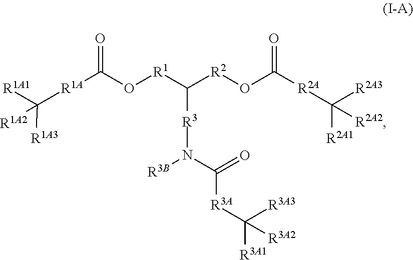

or a salt thereof, wherein:
R¹, R², and R³ are each independently a bond or C₁₋₃ alkylene;
R^{1A}, R^{2A}, and R^{3A} are each independently a bond or C₁₋₁₀ alkylene;
R^{1A1}, R^{1A2}, R^{1A3}, R^{2A1}, R^{2A2}, R^{2A3}, R^{3A1}, R^{3A2}, and R^{3A3} are each independently H, C₁₋₂₀ alkyl,
C₁₋₂₀ alkenyl, —(CH₂)₀₋₁₀C(O)OR^{a1}, or —(CH₂)₀₋₁₀OC(O)R^{a2};
R^{a1} and R^{a2} are each independently C₁₋₂₀ alkyl or C₁₋₂₀ alkenyl;
R^{3B} is

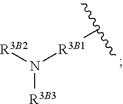

R^{3B1} is C₁₋₆ alkylene; and
R^{3B2} and R^{3B3} are each independently H, unsubstituted C₁₋₆ alkyl, or C₁₋₆ alkyl substituted with one or more substituents each independently selected from the group consisting of —OH and —O—(C₁₋₆ alkyl).

Any of the variables or substitutents provided herein is unsubstituted or substituted with one or more substituents. In some embodiments, any of the variables or substituents provided herein is optionally substituted. In some embodiments, any of the variables or substituents provided herein is optionally substituted with one or more substituents independently selected from the group consisting of —OR^{s1}, —NR^{s2}R^{s3}, —C(O)R^{s4}, —C(O)OR^{s5}, C(O)NR^{s6}R^{s7}, —OC(O)R^{s8}, —OC(O)OR^{s9}, —OC(O)NR^{s10}R¹¹, —NR^{s12}C(O)R^{s13} and —NR^{s14}C(O)OR^{s15}, wherein R^{s1}, R^{s2}, R^{s3}, R^{s4}, RSs, R^{s6}, R^{s7}, R^{s8}, R^{s9}, R^{s10}, R^{s11}, R^{s12}, R^{s13}, R^{s14}, and R^{s15} are each independently H, C₁₋₆ alkyl, C₃₋₁₀ cycloalkyl, C₆₋₁₄ aryl, 5- to 10-membered heteroaryl, or 3- to 10-membered heterocyclyl, each of which is optionally substituted.

In some embodiments, R¹, R², and R³ are each independently a bond or C₁₋₃ alkylene. In some embodiments, R¹, R², and R³ are each independently a bond or methylene. In some embodiments, R¹ and R² are each methylene and R³ is a bond. In some embodiments, R¹, R², and R³ are each methylene. In some embodiments, R¹, R², and R³ are each independently unsubstituted or substituted. In some embodiments, R¹, R², and R³ are unsubstituted.

In some embodiments, R^{1A}, R^{2A}, and R^{3A} are each independently a bond or C₁₋₁₀ alkylene. In some embodiments, R^{1A}, R^{2A}, and R^{3A} are each independently a bond or —(CH₂)₁₋₁₀—. In some embodiments, R^{1A} and R^{2A} are each independently a bond, —CH₂—, —(CH₂)₂—, —(CH₂)₃—, —(CH₂)₄—, —(CH₂)₅—, —(CH₂)₆—, —(CH₂)₇—, or —(CH₂)₈—. In some embodiments, R^{1A} and R^{2A} are each a bond, each —CH₂—, each —(CH₂)₂—, each —(CH₂)₃—, each —(CH₂)₄—, each —(CH₂)₅—, each —(CH₂)₆—, each —(CH₂)₇—, or each —(CH₂)₈—. In some embodiments, R^{1A} and R^{2A} are each independently a bond, —(CH₂)₂—, —(CH₂)₄—, —(CH₂)₆—, —(CH₂)₇—, or —(CH₂)₈—. In some embodiments, R^{1A} and R^{2A} are each a bond, each —(CH₂)₂—, each —(CH₂)₄—, each —(CH₂)₆—, each —(CH₂)₇—, or each —(CH₂)₈—. In some embodiments, R^{3A} is a bond, —CH₂—, —(CH₂)₂—, or —(CH₂)₇—. In some embodiments, R^{1A}, R^{2A}, and R^{3A} are each independently unsubstituted or substituted. In some embodiments, R^{1A}, R^{2A}, and R^{3A} are unsubstituted.

In some embodiments, R^{1A1}, R^{1A2}, R^{A3}, R^{2A1}, R^{2A}, R^{2A3}, R^{3A1}, R^{3A2}, and R^{3A3} are each independently H, C₁₋₂₀ alkyl, C₁₋₂₀ alkenyl, —(CH₂)₀₋₁₀C(O)OR^{a1}, or —(CH₂)₀₋₁₀OC(O)R^{a2}. In some embodiments, R^{1A1}, R^{1A2}, R^{A3}, R^{2A1}, R^{2A}, R^{2A3}, R^{3A1}, R^{3A2}, and R^{3A3} are each independently H, C₁₋₁₅ alkyl, —CH═CH—(C₁₋₁₅ alkyl), —CH═CH—CH₂—CH═CH—(C₁₋₁₀ alkyl), —(CH₂)₀₋₄C(O)OCH(C₁₋₁₀ alkyl)(C₁₋₁₅ alkyl), —(CH₂)₀₋₄OC(O)CH(C₁₋₁₀ alkyl)(C₁₋₁₅ alkyl), —(CH₂)₀₋₄C(O)OCH₂(C₁₋₁₅ alkyl), or —(CH₂)₀₋₄OC(O)CH₂(C₁₋₁₅ alkyl). In some embodiments, R^{1A1}, R^{1A2}, R^{1A3}, R^{2A1}, R^{2A2}, R^{2A3}, R^{3A1}, R^{3A}, R^{3A3}, R¹, R², R³, R^{1A}, R^{2A}, and R^{3A} are each independently unsubstituted or substituted. In some embodiments, R^{1A1}, R^{1A2}, R^{1A3}, R^{2A1}, R^{2A2}, R^{2A3}, R^{3A1}, R^{3A2}, R^{3A3}, R¹, R², R³, R^{1A}, R^{2A}, and R^{3A} are each unsubstituted. In some embodiments, R^{1A1}, R^{1A2}, R^{1A3}, R^{2A1}, R^{2A2}, R^{2A3}, R^{3A1}, R^{3A2}, and R^{3A3} are each independently unsubstituted or substituted. In some embodiments, R^{1A}, R^{1A2}, R^{1A3}, R^{2A1}, R^{2A2}, R^{2A3}, R^{3A1}, R^{3A2}, and R^{3A3} are each unsubstituted. In some embodiments, R¹, R², R³, R^{1A}, R^{2A}, and R^{3A} are each independently unsubstituted or substituted. In some embodiments, R¹, R², R³, R^{1A}, R^{2A} and R^{3A} are each unsubstituted. In some embodiments, R¹, R², and R³ are each unsubstituted.

In some embodiments, R^{3B1} is unsubstituted. In some embodiments, R^{3B1} is not substituted with oxo.

In some embodiments, R^{1A1} and R^{2A1} are each independently —CH═CH—(C₁₋₁₅ alkyl), —CH═CH—CH₂—CH═CH—(C₁₋₁₀ alkyl), —(CH₂)₀₋₄C(O)OCH(C₁₋₁₀ alkyl)(C₁₋₁₅ alkyl), or —(CH₂)₀₋₄OC(O)CH(C₁₋₁₀ alkyl)(C₁₋₁₅ alkyl); and R^{1A2}, R^{1A3}, R^{2A2}, and R^{2A3} are each H. In some embodiments, R^{1A1} and R^{2A1} are each —CH═CH—(C₁₋₁₅ alkyl), —CH═CH—CH₂—CH═CH—(C₁₋₁₀ alkyl), —(CH₂)₀₋₄C(O)OCH(C₁₋₁₀ alkyl)(C₁₋₁₅ alkyl), or —(CH₂)₀₋₄OC(O)CH(C₁₋₁₀ alkyl)(C₁₋₁₅ alkyl); and R^{1A2}, R^{1A3}, R^{2A2}, and R^{2A3} are each H. In some embodiments, R^{1A1} and R^{2A1} are each

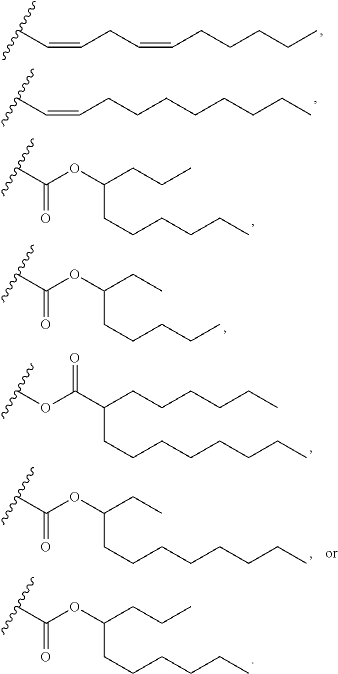

In some embodiments, R^{1A1} and R^{2A1} are each

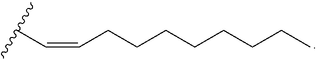 [SMILES: */C=C\CCCCCCCC]

In some embodiments, R^{1A}, R^{1A3}, R^{2A2}, and R^{2A3} are each H.

In some embodiments, R^{1A1} and R^{2A1} are each C₁₋₁₅ alkyl; R^{1A2} and R^{2A} are each C₁₋₁₅ alkyl; and R^{1A3} and R^{2A3} are each H. In some embodiments, R^{1A1} and R^{2A1} are each

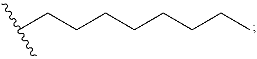 [SMILES: *CCCCCCCC]

and R^{1A2} and R^{2A2} are each

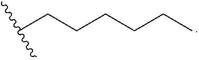 [SMILES: *CCCCCC]

In some embodiments, R^{1A3} and R^{2A3} are each H. In some embodiments, R^{1A} and R^{2A} are each a bond.

In some embodiments, R^{1A1} and R^{2A1} are each —(CH₂)₀₋₄OC(O)CH₂(C₁₋₁₅ alkyl); R^{2A1} and R^{2A2} are each —(CH₂)₀₋₄C(O)OCH₂(C₁₋₁₅ alkyl); and R^{1A3} and R^{2A3} are each H. In some embodiments, R^{1A1} and R^{2A1} are each

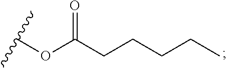 [SMILES: *OC(=O)CCCCC]

and R^{2A1} and R^{2A} are each

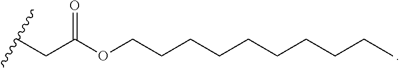 [SMILES: *CC(=O)OCCCCCCCCCC]

In some embodiments, R^{1A3} and R^{2A3} are each H. In some embodiments, R^{1A} and R^{2A} are each a bond.

In some embodiments, R^{1A1} and R^{2A1} are each —C(O)OCH₂(C₁₋₁₅ alkyl); R^{1A2} and R^{2A2} are each —(CH₂)₀₋₄C(O)OCH₂(C₁₋₁₅ alkyl); and R^{1A3} and R^{2A3} are each H. In some embodiments, R^{1A1} and R^{2A1} are each

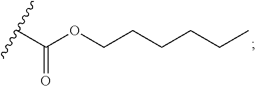 [SMILES: *C(=O)OCCCCCC]

and R^{1A2} and R^{2A2} are each

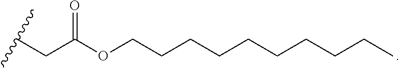 [SMILES: *CC(=O)OCCCCCCCCCC]

In some embodiments, R^{1A1} and R^{2A1} are each

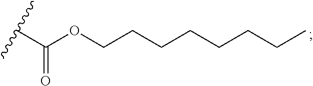 [SMILES: *C(=O)OCCCCCCCC]

and R^{2A1} and R^{2A2} are each

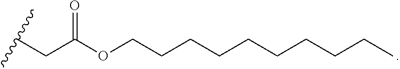 [SMILES: *CC(=O)OCCCCCCCCCC]

In some embodiments, R^{1A3} and R^{2A3} are each H. In some embodiments, R^{1A} and R^{2A} are each a bond.

In some embodiments, R^{3A1}, R^{3A2}, and R^{3A3} are each independently H, C₁₋₁₅ alkyl, —(CH₂)₀₋₄C(O)OCH(C₁₋₅ alkyl)(C₁₋₁₀ alkyl), —(CH₂)₀₋₄OC(O)CH(C₁₋₅ alkyl)(C₁₋₁₀ alkyl), —(CH₂)₀₋₄C(O)OCH₂(C₁₋₁₀ alkyl), or —(CH₂)₀₋₄OC(O)CH₂(C₁₋₁₀ alkyl).

In some embodiments, R^{3A1} and R^{3A2} are each independently C₁₋₁₅ alkyl; and R^{3A3} is H. In some embodiments, R^{3A1} and R^{3A2} are each independently ethyl, propyl, butyl, pentyl, hexyl, or heptyl. In some embodiments, R^{3A1} and R^{3A2} are each independently ethyl,

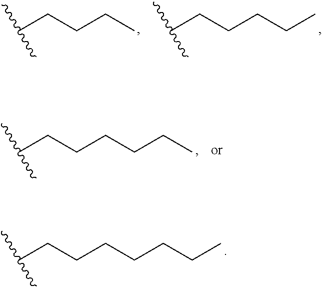

In some embodiments, R^{3A3} is H. In some embodiments, R^{3A} is a bond.

In some embodiments, R^{3A1} is C₁₋₁₅ alkyl; and R^{3A2} and R^{3A3} are each H. In some embodiments, R^{3A1} is

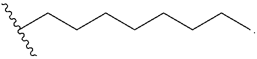 [SMILES: *CCCCCCCC]

In some embodiments, R^{3A2} and R^{3A3} are each H. In some embodiments, R^{3A} is a bond.

In some embodiments, R^{3A1} is —C(O)OCH(C₁₋₅ alkyl)(C₁₋₁₀ alkyl); and R^{3A2} and R^{3A3} are each H. In some embodiments, R^{3A1} is or

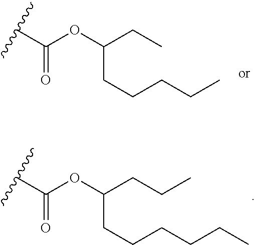 [SMILES: *C(=O)OC(CC)CCCCC ; *C(=O)OC(CCC)CCCCCC]

In some embodiments, R^{3A1} is

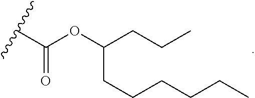 [SMILES: *C(=O)OC(CCC)CCCCCC]

In some embodiments, R^{3A} is ethylene or —(CH₂)₂—. In some embodiments, R^{3A2} and R^{3A3} are each H.

In some embodiments, R^{3A1} is —(CH₂)₀₋₄OC(O)CH₂(C₁₋₁₀ alkyl); R^{3A2} is —(CH₂)₀₋₄(O)OCH₂(C₁₋₁₀ alkyl); and R^{3A3} is H. In some embodiments, R^{3A1} is

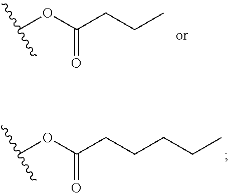

### and R^{3A2} is

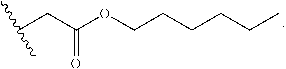 [SMILES: *CC(=O)OCCCCCC]

In some embodiments, R^{3A3} is H. In some embodiments, R^{3A} is a bond.

In some embodiments, R^{3A1} is —(CH₂)₀₋₄C(O)OCH₂(C₁₋₁₀ alkyl); R^{3A2} is —(CH₂)₀₋₄C(O)OCH₂(C₁₋₁₀ alkyl); and R^{3A3} is H. In some embodiments, R^{3A1} is

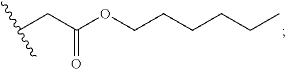 [SMILES: *CC(=O)OCCCCCC]

### and R^{3A2} is or

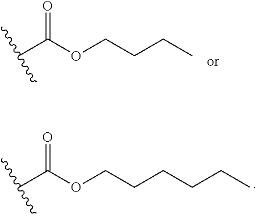

In some embodiments, R^{3A3} is H. In some embodiments, R^{3A} is a bond.

In some embodiments, R^{3A1}, R^{3A2}, and R^{3A3} are each H.

R^{a1} and R^{a2} are each independently C₁₋₂₀ alkyl or C₁₋₂₀ alkenyl. In some embodiments, R^{a1} and R^{a2} are each independently —(CH₂)₀₋₁₅CH₃ or —CH(C₁₋₁₀ alkyl)(C₁₋₁₅ alkyl). In some embodiments, R^{a1} and R^{a2} are each independently

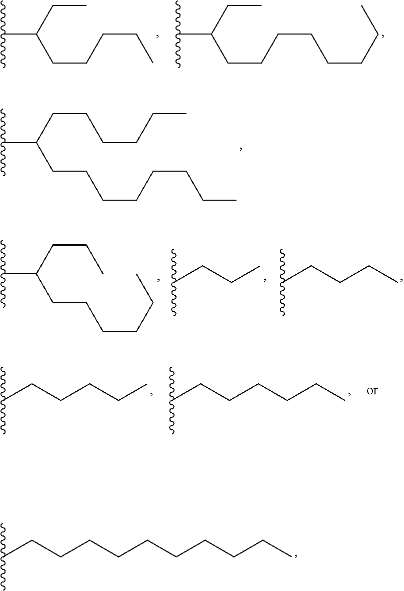

each of which is optionally substituted. In some embodiments, R^{a1} and R^{a2} are each independently unsubstituted or substituted. In some embodiments, R^{a1} and R^{a2} are unsubstituted.

In some embodiments, R^{3B} is

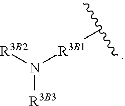

In some embodiments, R^{3B} is H. In some embodiments, R^{3B} is unsubstituted or substituted. In some embodiments, R^{3B} is unsubstituted.

In some embodiments, R^{3B1} is C₁₋₆ alkylene. In some embodiments, R^{3B1} is ethylene or propylene. In some embodiments, R^{3B1} is unsubstituted or substituted. In some embodiments, R^{3B1} is optionally substituted.

In some embodiments, R^{3B2} and R^{3B3} are each independently and optionally substituted. In some embodiments, R^{3B2} and R^{3B3} are each independently H or C₁₋₆ alkyl optionally substituted with one or more substituents each independently selected from the group consisting of —OH and —O—(C₁₋₆ alkyl). In some embodiments, R^{3B2} and R^{3B3} are each independently H or C₁₋₆ alkyl optionally substituted with one or more substituents independently selected from the group consisting of —OR^{s1}, —NR^{s2}R^{s3}, —C(O)R^{s4}, —C(O)OR^{s5}, C(O)NR^{s6}R^{s7}, —OC(O)R^{s8}, —OC(O)OR^{s9}, —OC(O)NR^{s10}R¹¹, —NR^{s12}C(O)R^{s13} and —NR^{s14}C(O)OR^{s15}, wherein R^{s1}, R^{s2}, R^{s3}, R^{s4}, RSs, R^{s6}, R^{s7}, R^{s8}, R^{s9}, R^{s10}, R^{s11}, R^{s12}, R^{s13}, R^{s14}, and R^{s15} are each independently H, C₁₋₆ alkyl, C₃₋₁₀ cycloalkyl, C₆₋₁₄ aryl, 5- to 10-membered heteroaryl, or 3- to 10-membered heterocyclyl, each of which is optionally substituted. In some embodiments, R^{3B2} and R^{3B3} are each independently H, methyl, ethyl, propyl, butyl, or pentyl, each of which is optionally substituted with one or more substituents each independently selected from the group consisting of —OH and —O—(C₁₋₆ alkyl). In some embodiments, R^{3B2} and R^{3B3} are each independently methyl or ethyl, each optionally substituted with one or more —OH. In some embodiments, R^{3B2} and R^{3B3} are each methyl or each ethyl, each optionally substituted with one or more —OH. In some embodiments, R^{3B2} and R^{3B3} are each unsubstituted methyl.

In some embodiments,

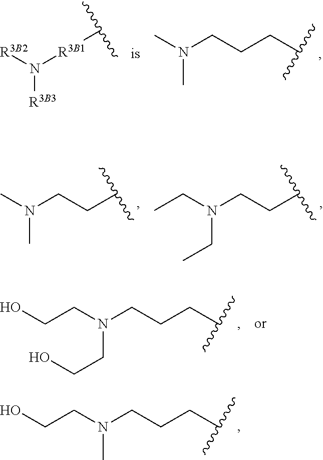

each of which is optionally substituted.

In one aspect, the present invention provides a compound represented by Formula (Ia):

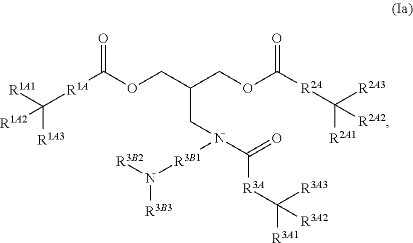

or a salt thereof, wherein R^{1A}, R^{2A}, R^{3A}, R^{1A1}, R^{1A2}, R^{1A3}, R^{2A1}, R^{2A2}, R^{2A3}, R^{3A1}, R^{3A}, R^{3A3}, R^{3B1}, R^{3B2}, and R^{3B3} are as defined for Formula (I) or any variation or embodiment thereof.

In one aspect, the present invention provides a compound represented by Formula (Ib):

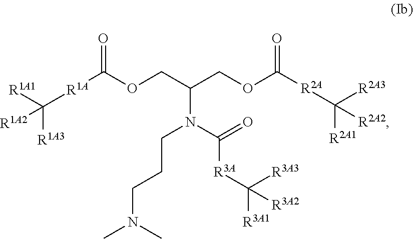

or a salt thereof, wherein R^{1A}, R^{2A}, R^{3A}, R^{1A1}, R^{1A2}, R^{1A3}, R^{2A1}, R^{2A}, R^{2A3}, R^{3A1}, R^{3A}, and R^{3A3} are as defined for Formula (I) or any variation or embodiment thereof.

### III. Lipid-Immune Cell Targeting Group Conjugates

As discussed herein, the LNPs may be targeted to a particular cell type, e.g., an immune cell, e.g., a T cell, B cell, or natural killer (NK) cell. This can be accomplished by using one or more of the lipids described herein. Furthermore, targeting can be enhanced by including a targeting group at a solvent accessible surface of an LNP particle. For example, targeting groups may include a member of a specific binding pair, e.g., an antibody-antigen pair, a ligand-receptor pair, etc. In certain embodiments, the targeting group is an antibody. Targeting can be implemented, for example, by using lipid-immune cell targeting group conjugates described herein.

Optionally, the targeting moiety is an antibody fragment without an Fc component. Previous attempts to target circulating immune cells with LNPs have employed full antibodies (WO 2016/189532 A1). Liposomes or lipid based particles with conjugated full antibodies clear more quickly from the circulation due to engagement of the Fc, reducing their potential for reaching the target cell of interest (Harding et al. (1997) Biochim Biophys. Acta 1327, 181-192; Sapra et al. (2004) Clin Cancer Res 10, 1100-1111; Aragnol et al., (1986) Proc Natl Acad Sci USA 83, 2699-2703). Liposomes targeted with antibody fragments retain their long circulating properties, like those targeted to EGFR (Mamot et al., (2005) Cancer Res 65, 11631-11638), ErbB2 (Park et al. (2002) Clin Cancer Res 8, 1172-1181), or EphA2 (Kamoun et al., 2019 Nat. Biomed. Eng 3, 264-280). In addition, lipid based carriers can be prepared using a micellar insertion process that allows for the nearly quantitative incorporation of the antibody conjugation following its separate manufacturing (Nellis et al. (2005) Biotechnol Prog 21, 221-232), compared to a highly inefficient insertion when conjugating full IgGs (Ishida et al. (1999) FEBS Lett. 460, 129-133) or the need to complete conjugation directly on an intact LNP (WO 2016/189532 A1). scFv, Fab, or V_{HH} fragments can also be directly conjugated to activated PEG-lipids to make insertable conjugates.

In some embodiments, PEG-(lipid) is equivalent to (lipid)-PEG.

In certain embodiments, a targeting group may be a surface-bound antibody or surface bound antigen binding fragment thereof, which can permit tuning of cell targeting specificity. This is especially useful since highly specific antibodies can be raised against an epitope of interest for the desired targeting site. In one embodiment, multiple different antibodies can be incorporated into, and presented at the surface of an LNP, where each antibody binds to different epitopes on the same antigen or different epitopes on different antigens. Such approaches can increase the avidity and specificity of targeting interactions to a particular target cell.

A targeting group or combination of targeting groups can be selected based on the desired localization, function, or structural features of a given target cell. For example, in order to target a T-cell, T-cell population or T-cell subpopulation, one or more antibodies or antigen binding fragments or antigen binding derivatives thereof may be selected that target a T-cell, such as via a T-cell surface antigen. Exemplary T-cell surface antigens include, but are not limited to, for example, CD2, CD3, CD4, CD5, CD7, CD8, CD28, CD39, CD69, CD103, CD137, CD45, T-cell receptor (TCR) β, TCR-α, TCR-α/β, TCR-γ/δ, PD1, CTLA4, TIM3, LAG3, CD18, IL-2 receptor, CD11a, GL7, TLR2, TLR4, TLR5 and IL-15 receptor. In order to target an NK cell, or NK cell population, one or more antibodies, antigen binding fragments or antigen binding derivatives thereof may be selected that target an NK cell such as via a NK cell surface antigen. Exemplary NK cell surface antigens include, but are not limited to, CD48, CD56, CD85a, CD85c, CD85d, CD85e, CD85f, CD85i, CD85j, CD158b2, CD161, CD244, CD16a, CD16b, IL-2 receptor, CD27, CD28, CD48, CD69, CD70, CD86, CD112, CD122, CD155, CD161, CD244, CD266, CD314/NKG2D, CD336/NKP44, CD337/NKP30. In order to target a B cell or B cell population, one or more antibodies, antigen binding fragments or antigen binding derivatives thereof may be selected that target a B cell such as via a B cell antigen. Exemplary B cell antigens include, but are not limited to, CD19 for all B cells except plasma cells, CD19, CD25, and CD30 for activated B cells, CD27, CD38, CD78, CD138, and CD319 for plasma cells, CD20, CD27, CD40, CD80 and PDL-2 for memory cells, Notch2, CD1, CD21, and CD27 for marginal zone B cells, CD21, CD22, and CD23 for follicular B cells, and CD1, CD5, CD21, CD24, and TLR4 for regulatory B cells.

In certain embodiments, targeting can be implemented, for example, by using lipid-immune cell targeting group conjugates described herein. Exemplary lipid-immune cell targeting group conjugates can include compounds of Formula (II), [Lipid]-[optional linker]-[immune cell targeting group, e.g., T-cell targeting molecule, e.g., anti-CD2 antibody, anti-CD3 antibody, anti-CD7 antibody, or anti-CD8 antibody](Formula II).

In some embodiments, the immune cell targeting group is a polypeptide, and the lipid is conjugated to the N-terminus, C-terminus, or anywhere in the middle part of the polypeptide.

In certain embodiments, the targeting group or targeting molecule is a T-cell targeting agent, for example, an antibody, that binds to a T-cell antigen selected from the group consisting of CD2, CD3, CD4, CD5, CD7, CD8, CD28, CD137, CD45, T-cell receptor (TCR)β, TCR-α, TCR-α/β, TCR-γ/δ, PD1, CTLA4, TIM3, LAG3, CD18, IL-2 receptor, CD11a, TLR2, TLR4, TLR5, IL-7 receptor, or IL-15 receptor. In certain embodiments, the T cell antigen may be CD2, and the targeting group can be, for example, an anti-CD2 antibody. In certain embodiments, the T cell antigen may be CD3, and the targeting group can be, for example, an anti-CD3 antibody. In certain embodiments, the T cell antigen may be CD4, and the targeting group can be, for example, an anti-CD4 antibody. In certain embodiments, the T cell antigen may be CD5, and the targeting group can be, for example, an anti-CD5 antibody. In certain embodiments, the T cell antigen may be CD7, and the targeting group can be, for example, an anti-CD7 antibody. In certain embodiments, the T cell antigen may be CD8, and the targeting group can be, for example, an anti-CD8 antibody. In certain embodiments, the T cell antigen may be TCR β, and the targeting group can be, for example, an anti-TCR β antibody. In some embodiments, the antibody is a human or humanized antibody.

An exemplary CD2 binding agent can be an antibody selected from the group consisting of 9.6 (https://academic.oup.com/intimm/article/10/12/1863/744536), 9-1 (https://academic.oup.com/intimm/article/10/12/1863/744536), TS2/18.1.1 (ATCC HB-195), Lo-CD2b (ATCC PTA-802), Lo-CD2a/BTI-322 (U.S. Pat. No. 6,849,258B1), Sipilzumab/MEDI-507 (U.S. Pat. No. 6,849,258B1/en), 35.1 (ATCC HB-222), OKT11 (ATCC CRL-8027), RPA-2.1 (PCT Publication WO2020023559A1), AF1856 (R&D Systems), MAB18562 (R&D Systems), MAB18561 (R&D Systems), MAB1856 (R&D Systems), PAB30359 (Abnova Corporation), 10299-1 (Abnova Corporation), and antigen binding fragments thereof. In certain embodiments, the binding agent comprises a heavy chain variable domain (V_{H}) and a light chain variable domain (V_{L}) of an antibody selected from the group consisting of AF1856 (R&D Systems), MAB18562 (R&D Systems), MAB18561 (R&D Systems), MAB1856 (R&D Systems), PAB30359 (Abnova Corporation), and 10299-1 (Abnova Corporation). In certain embodiments, the binding agent comprises the heavy chain CDR₁, CDR₂, and CDR₃ and the light chain CDR₁, CDR₂, and CDR₃, determined under Kabat (see, Kabat et al., (1991) Sequences of Proteins of Immunological Interest, NIH Publication No. 91-3242, Bethesda), Chothia (see, e.g., Chothia C & Lesk A M, (1987), J. MOL. BIOL. 196: 901-917), MacCallum (see, MacCallum R M et al., (1996) J. MOL. BIOL. 262: 732-745), or any other CDR determination method known in the art, of the V_{H} and V_{L} sequences of an antibody selected from the group consisting of AF1856 (R&D Systems), MAB18562 (R&D Systems), MAB18561 (R&D Systems), MAB1856 (R&D Systems), PAB30359 (Abnova Corporation), and 10299-1 (Abnova Corporation).

An exemplary CD2 binding agent can also be selected from antibodies or antibody fragments employing CDRs of clones 9.6, 9-1, TS2/18.1.1, Lo-CD2b, Lo-CD2a, BTI-322, sipilzumab, 35.1, OKT11, RPA-2.1, SQB-3.21, LT2, TS1/8, UT329, 4F22, OX-34, UQ2/42, MU3, U7.4, NFN-76, or MOM-181-4-F(E).

An exemplary CD3 binding agent (CD3γ/δ/ε, CD3γ, CD3δ, CD3γ/ε, CD3δ/ε, or CD3ε) can be an antibody selected from the group consisting of MEM-57 (CD3γ/δ/Σ, EnzoLife Sciences), MAB100 (CD3ε, R&D Systems), CD3-H5 (CD3ε, Abnova Corporation), CD3-12 (CD3ε, Cell Signaling Technology), LE-CD3 (CD3ε, Santa Cruz Biotechnology, Inc.), NBP1-31250 (CD3γ, Novus Biologicals), 16669-1-AP (CD3δ, Invitrogen) and antigen binding fragments thereof. In certain embodiments, the binding agent comprises a V_{H} domain and a V_{L} domain of an antibody selected from the group consisting of MEM-57 (CD3γ/δ/Σ, EnzoLife Sciences), MAB100 (CD3ε, R&D Systems), CD3-H5 (CD3ε, Abnova Corporation), CD3-12 (CD3ε, Cell Signaling Technology), LE-CD3 (CD3ε, Santa Cruz Biotechnology, Inc.), NBP1-31250 (CD3γ, Novus Biologicals), and 16669-1-AP (CD3δ, Invitrogen). In certain embodiments, the binding agent comprises the heavy chain CDR₁, CDR₂, and CDR₃ and the light chain CDR₁, CDR₂, and CDR₃, determined under Kabat (see, Kabat et al., (1991) Sequences of Proteins of Immunological Interest, NIH Publication No. 91-3242, Bethesda), Chothia (see, e.g., Chothia C & Lesk A M, (1987), J. MOL. BIOL. 196: 901-917), MacCallum (see, MacCallum R M et al., (1996) J. MOL. BIOL. 262: 732-745), or any other CDR determination method known in the art, of the V_{H} and V_{L} sequences of an antibody selected from the group consisting of MEM-57 (CD3γ/δ/Σ, EnzoLife Sciences), MAB100 (CD3ε, R&D Systems), CD3-H5 (CD3ε, Abnova Corporation), CD3-12 (CD3ε, Cell Signaling Technology), LE-CD3 (CD3ε, Santa Cruz Biotechnology, Inc.), NBP1-31250 (CD3γ, Novus Biologicals), and 16669-1-AP (CD3δ, Invitrogen).

An exemplary CD3 binding agent can also be selected from antibodies or antibody fragments employing CDRs of clones hsp34, OKT-3, UCHT1, 38.1, HIT3a, RFT8, SK7, BC3, SP34-2, HU291, TRX4, Catumaxomab, teplizumab, 3-106, 3-114, 3-148, 3-190, 3-271, 3-550, 4-10, 4-48, H2C, F12Q, I2C, SP7, 3F3A1, CD3-12, 301, RIV9, JB38-29, JE17-74, GT0013, 4E2, 7A4, 4D10A6, SPV-T3b, M2AB, ICO-90, 30A1 or Hu38E4.v1 (US Patent Application 20200299409A1), REGN5458 (US Patent Application 20200024356A1), Blinatumomab (https://go.drugbank.com/drugs/DB09052/polypeptide_sequences.fasta). In some embodiments, the conjugate comprises a Fab, wherein the Fab comprises (a) a heavy chain fragment comprising the amino acid sequence of SEQ ID NO: 1 and a light chain fragment comprising the amino acid sequence of SEQ ID NO: 2 or 3.

An exemplary CD4 binding agent can be an antibody selected from the group consisting of Ibalizumab (https://www.genome.jp/dbget-bin/www_bget?D09575), AF1856 (R&D Systems), MAB554 (R&D Systems), BF0174 (Affinity Biosciences), PAB31115 (Abnova Corporation), CAL4 (Abcam), and antigen binding fragments thereof. In certain embodiments, the binding agent comprises a V_{H} domain and a V_{L} domain of an antibody selected from the group consisting of AF1856 (R&D Systems), MAB554 (R&D Systems), BF0174 (Affinity Biosciences), PAB31115 (Abnova Corporation), and CAL4 (Abcam). In certain embodiments, the binding agent comprises the heavy chain CDR₁, CDR₂, and CDR₃ and the light chain CDR₁, CDR₂, and CDR₃, determined under Kabat (see, Kabat et al., (1991) Sequences of Proteins of Immunological Interest, NIH Publication No. 91-3242, Bethesda), Chothia (see, e.g., Chothia C & Lesk A M, (1987), J. MOL. BIOL. 196: 901-917), MacCallum (see, MacCallum R M et al., (1996) J. MOL. BIOL. 262: 732-745), or any other CDR determination method known in the art, of the V_{H} and V_{L} sequences of an antibody selected from the group consisting of AF1856 (R&D Systems), MAB554 (R&D Systems), BF0174 (Affinity Biosciences), PAB31115 (Abnova Corporation), and CAL4 (Abcam).

An exemplary CD4 binding agent can also be selected from antibodies or antibody fragments employing CDRs of clones Ibalizumab, OKT4, RPA-T4, S3.5, SK3, N1UGO, RIV6, OTI18E3, MEM-241, B486A1, RFT-4 g, 7E14, MDX.2, MEM-115, MEM-16, ICO-86, Edu-2, or ilbalizumab.

An exemplary CD5 binding agent can be an antibody selected from the group consisting of He3, MAB1636 (R&D Systems), AF1636 (R&D Systems), MAB115 (R&D Systems), C_{5/473}+CD5/54/F6 (Abcam), CD5/54/F6 (Abcam), 65152 (Proteintech), and antigen binding fragments thereof. In some embodiments, the binding agent comprises a V_{H} domain and a V_{L} of an antibody selected from the group consisting of MAB1636 (R&D Systems), AF1636 (R&D Systems), MAB115 (R&D Systems), C_{5/473}+CD5/54/F6 (Abcam), CD5/54/F6 (Abcam), and 65152 (Proteintech). In certain embodiments, the binding agent comprises the heavy chain CDR₁, CDR₂, and CDR₃ and the light chain CDR₁, CDR₂, and CDR₃, determined under Kabat (see, Kabat et al., (1991) Sequences of Proteins of Immunological Interest, NIH Publication No. 91-3242, Bethesda), Chothia (see, e.g., Chothia C & Lesk A M, (1987), J. MOL. BIOL. 196: 901-917), MacCallum (see, MacCallum R M et al., (1996) J. MOL. BIOL. 262: 732-745), or any other CDR determination method known in the art, of the V_{H} and V_{L} sequences of an antibody selected from the group consisting of MAB1636 (R&D Systems), AF1636 (R&D Systems), MAB115 (R&D Systems), C_{5/473}+CD5/54/F6 (Abcam), CD5/54/F6 (Abcam), and 65152 (Proteintech).

An exemplary CD5 binding agent can also be selected from antibodies or antibody fragments employing CDRs of clones of zolimomab, 5D7, L17F12, and UCHT2, 1D8, 3121, 4H10, 8J23, 5O4, 4H2, 5G2, 8G8, 6M4, 2E3, 4E24, 4F10, 7J9, 7P9, 8E24, 6L18, 7H7, 1E7, 8J21, 7I11, 8M9, 1P21, 2H11, 3M22, 5M6, 5H8, 7I19, 1A2, 8E15, 8C10, 3P16, 4F3, 5M24, 5O24, 7B16, 1E8, 2H16, BLa1, 1804, DK23, Cris1, MEM-32, H65, 4C₇, OX-19, Leu-1, 53-7.3, 4H8E6, T101, EP2952, D-9, H-3, HK231, N-20, Y2/178, H-300, CD5/54/F6, Q-20, CC17, MOM-18539-S(P), or MOM-18885-S(P).

An exemplary CD7 binding agent can be an antibody selected from the group consisting of MAB7579 (R&D Systems), AF7579 (R&D Systems), EPR22065 (Abcam), 1G10D8 (Proteintech), NBP2-32097 (Novus Biologicals), NBP2-38440 (Novus Biologicals), and antigen binding fragments thereof. In certain embodiments, the binding agent comprises a V_{H} domain and a V_{L} of an antibody selected from the group consisting of MAB7579 (R&D Systems), AF7579 (R&D Systems), EPR22065 (Abcam), 1G10D8 (Proteintech), NBP2-32097 (Novus Biologicals), and NBP2-38440 (Novus Biologicals). In certain embodiments, the binding agent comprises the heavy chain CDR₁, CDR₂, and CDR₃ and the light chain CDR₁, CDR₂, and CDR₃, determined under Kabat (see, Kabat et al., (1991) Sequences of Proteins of Immunological Interest, NIH Publication No. 91-3242, Bethesda), Chothia (see, e.g., Chothia C & Lesk A M, (1987), J. MOL. BIOL. 196: 901-917), MacCallum (see, MacCallum R M et al., (1996) J. MOL. BIOL. 262: 732-745), or any other CDR determination method known in the art, of the V_{H} and V_{L} sequences of an antibody selected from the group consisting of MAB7579 (R&D Systems), AF7579 (R&D Systems), EPR22065 (Abcam), 1G10D8 (Proteintech), NBP2-32097 (Novus Biologicals), and NBP2-38440 (Novus Biologicals).

An exemplary CD7 binding agent can also be selected from antibodies or antibody fragments employing CDRs of clones TH-69, 3Afl1, T3-3A1, 124-1D1, 3Alf, CD7-6B7, or V_{HH}6.

An exemplary CD8 (CD8α, CD8α/α, CD8α/β or CD8β) binding agent can be an antibody selected from the group consisting of 2.43 (Invitrogen), Du CD8-1 (CD8α, Invitrogen), 9358-CD (CD8α/β, R&D Systems), MAB116 (CD8α, R&D Systems), ab4055 (CD8α, Abcam), C_{8/144}B (CD8α, Novus Biologicals), YTS105.18 (CD8α, Novus Biologicals), TRX2 (https://patents.justia.com/patent/20170198045), and antigen binding fragments thereof. In certain embodiments, the binding agent comprises a V_{H} domain and a V_{L} domain of an antibody selected from the group consisting of 2.43 (Invitrogen), 51.1 (ATCC HB-230), Du CD8-1 (CD8α, Invitrogen), 9358-CD (CD8α/β, R&D Systems), MAB116 (CD8α, R&D Systems), ab4055 (CD8α, Abcam), C_{8/144}B (CD8α, Novus Biologicals), and YTS105.18 (CD8α, Novus Biologicals). In certain embodiments, the binding agent comprises the heavy chain CDR₁, CDR₂, and CDR₃ and the light chain CDR₁, CDR₂, and CDR₃, determined under Kabat (see, Kabat et al., (1991) Sequences of Proteins of Immunological Interest, NIH Publication No. 91-3242, Bethesda), Chothia (see, e.g., Chothia C & Lesk A M, (1987), J. MOL. BIOL. 196: 901-917), MacCallum (see, MacCallum R M et al., (1996) J. MOL. BIOL. 262: 732-745), or any other CDR determination method known in the art, of the V_{H} and V_{L} sequences of an antibody selected from the group consisting of 2.43 (Invitrogen), Du CD8-1 (CD8α, Invitrogen), 9358-CD (CD8α/β, R&D Systems), MAB116 (CD8α, R&D Systems), ab4055 (CD8α, Abcam), C_{8/144}B (CD8α, Novus Biologicals), and YTS105.18 (CD8α, Novus Biologicals).

An exemplary CD8 binding agent can also be selected from antibodies or antibody fragments employing CDRs of clones OKT-8, 51.1, S6F1, TRX2, and UCHT4, SP16, 3B5, C₈₋₁₄₄B, HIT8a, RAVB3, LT8, 17D8, MEM-31, MEM-87, RIV11, DK-25, YTC141.1HL, or YTC182.20. In some embodiments, the conjugate comprises a Fab, wherein the Fab comprises a heavy chain fragment comprising the amino acid sequence of SEQ ID NO: 6 and a light chain fragment comprising the amino acid sequence of SEQ ID NO: 7.

An exemplary CD137 binding agent can be selected from antibodies or antibody fragments employing CDRs of clones 4B4-1, P566, or Urelumab. An exemplary CD28 binding agent can be selected from antibodies or antibody fragments employing CDRs of clone TAB08. An exemplary CD45 binding agent can be selected from antibodies or antibody fragments employing CDRs of clones BC8, 9.4, 4B2, Tu116, or GAP8.3. An exemplary CD18 binding agent can be selected from antibodies or antibody fragments employing CDRs of clones 1B4, TS1/18, MEM-48, YFC118-3, TA-4, MEM-148, or R3-3, 24. An exemplary CD11a binding agent can be selected from antibodies or antibody fragments employing CDRs of clone MHM24 or Efalizumab. An exemplary IL-2 receptor binding agent can be selected from of antibodies or antibody fragments employing CDRs of clones YTH 906.9HL, IL2R.1, BC96, B-B10, 216, MEM-181, ITYV, MEM-140, ICO-105, Daclizumab, or from the group consisting of IL2 or fragments of IL2. An exemplary IL-15R binding agent can be selected from antibodies or antibody fragments employing CDRs of clones JM7A4, or OTI3D5, or from the group consisting of IL15 or fragments of IL15. An exemplary TLR2 binding agent can be selected from antibodies or antibody fragments employing CDRs of clones JM22-41, TL2.1, 11G7, or TLR2.45. An exemplary TLR4 binding agent can be selected from antibodies or antibody fragments employing CDRs of clones HTA125, or 76B357-1. An exemplary TLR5 binding agent can be selected from antibodies or antibody fragments employing CDRs of clones 85B152-5, or 9D759-2. An exemplary GL7 binding agent can be selected from antibodies or antibody fragments employing CDRs of clone GL7.

An exemplary PD1 binding agent can be selected from antibodies or antibody fragments employing CDRs of clones MIH4, J116, J150, OTIB11, OTI17B10, OTI3A1, or OTI16D4. In addition, exemplary anti-PD-1 antibodies are described, for example, in U.S. Pat. Nos. 8,952,136, 8,779,105, 8,008,449, 8,741,295, 9,205,148, 9,181,342, 9,102,728, 9,102,727, 8,952,136, 8,927,697, 8,900,587, 8,735,553, and 7,488,802. Exemplary anti-PD-1 antibodies include, for example, nivolumab (Opdivo®, Bristol-Myers Squibb Co.), pembrolizumab (Keytruda®, Merck Sharp & Dohme Corp.), PDR001 (Novartis Pharmaceuticals), and pidilizumab (CT-011, Cure Tech). Exemplary anti-PD-L1 antibodies are described, for example, in U.S. Pat. Nos. 9,273,135, 7,943,743, 9,175,082, 8,741,295, 8,552,154, and 8,217,149. Exemplary anti-PD-L1 antibodies include, for example, atezolizumab (Tecentriq®, Genentech), durvalumab (AstraZeneca), MEDI4736, avelumab, and BMS 936559 (Bristol Myers Squibb Co.).

An exemplary CTLA-4 binding agent can be selected from antibodies or antibody fragments employing CDRs of clones ER4.7G.11 [7G11], OTI9G4, OTI9F3, OTI3A5, A3.4H2.H12, 14D3, OTI3A12, OTI1A11, OTI1E8, OTI3B11, OTI3D2, OTI1OC8, OTI2E9, OTI6F1, OTI7D3, OTI85B, OTI12C₆. Exemplary anti-CTLA-4 antibodies are described in U.S. Pat. Nos. 6,984,720, 6,682,736, 7,311,910; 7,307,064, 7,109,003, 7,132,281, 6,207,156, 7,807,797, 7,824,679, 8,143,379, 8,263,073, 8,318,916, 8,017,114, 8,784,815, and 8,883,984, International (PCT) Publication Nos. WO98/42752, WO00/37504, and WO01/14424, and European Patent No. EP 1212422 B1. Exemplary CTLA-4 antibodies include ipilimumab or tremelimumab.

An exemplary TCR β binding agent can be an antibody selected from the group consisting of H57-597 (Invitrogen), 8A3 (Novus Biologicals), R⁷³ (TCRα/β, Abcam), E6Z3S (TRBC1/TCRβ, Cell Signaling Technology), and antigen binding fragments thereof. In certain embodiments, the binding agent comprises a V_{H} domain and a V_{L} of an antibody selected from the group consisting of H57-597 (Invitrogen), 8A3 (Novus Biologicals), R⁷³ (TCRα/β, Abcam), and E6Z3S (TRBC1/TCRβ, Cell Signaling Technology). In certain embodiments, the binding agent comprises the heavy chain CDR₁, CDR₂, and CDR₃ and the light chain CDR₁, CDR₂, and CDR₃, determined under Kabat (see, Kabat et al., (1991) Sequences of Proteins of Immunological Interest, NIH Publication No. 91-3242, Bethesda), Chothia (see, e.g., Chothia C & Lesk A M, (1987), J. MOL. BIOL. 196: 901-917), MacCallum (see, MacCallum R M et al., (1996) J. MOL. BIOL. 262: 732-745), or any other CDR determination method known in the art, of the V_{H} and V_{L} sequences of an antibody selected from the group consisting of H57-597 (Invitrogen), 8A3 (Novus Biologicals), R73 (TCRα/β, Abcam), and E6Z3S (TRBC1/TCRβ, Cell Signaling Technology).

An exemplary CD137 binding agent can be selected from antibodies or antibody fragments employing CDRs of clones 4B4-1, P566, or Urelumab.

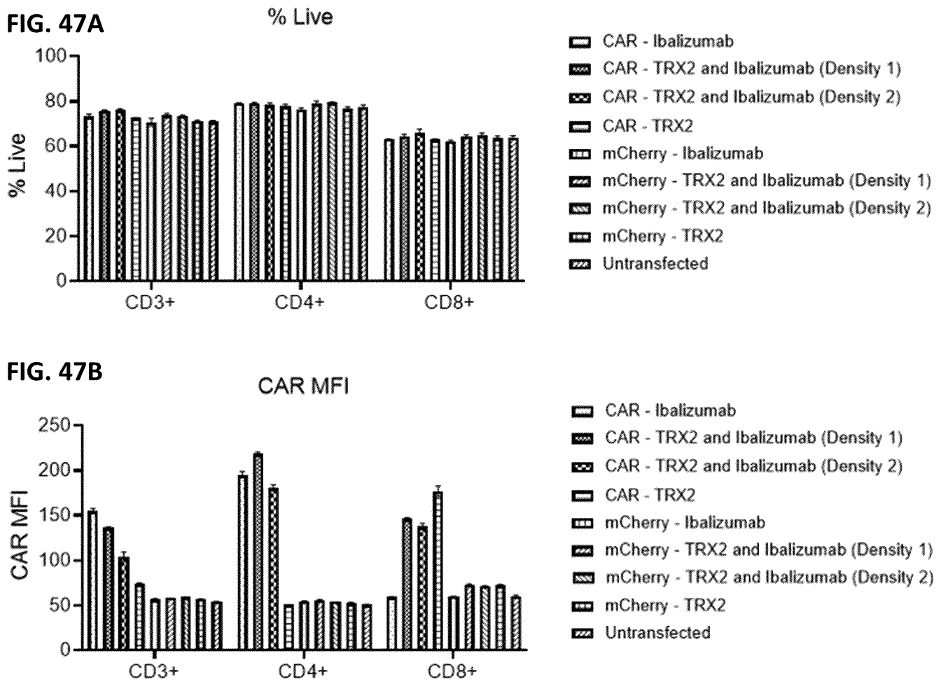

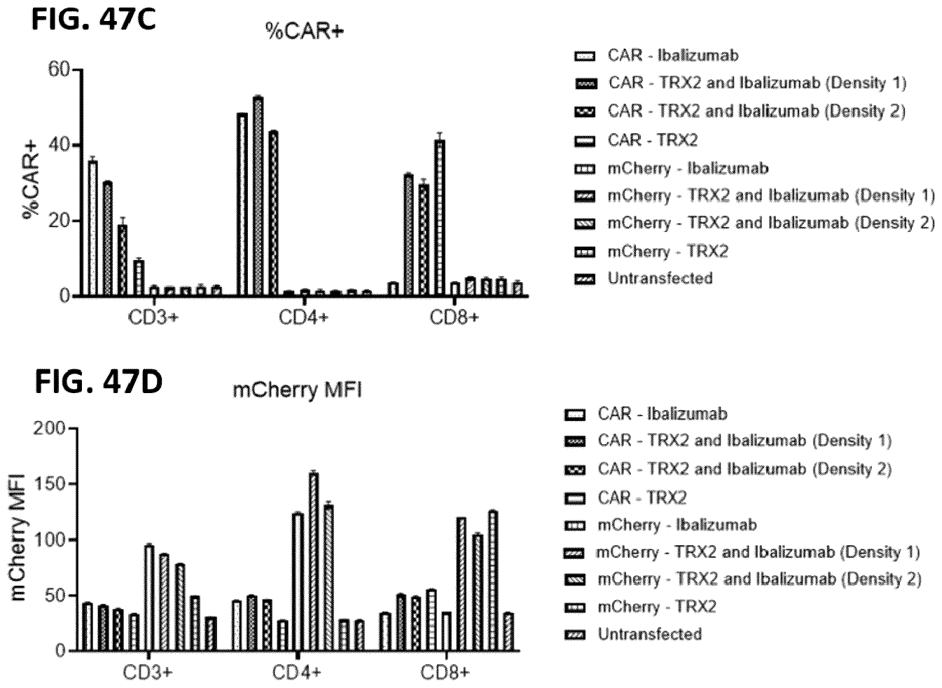

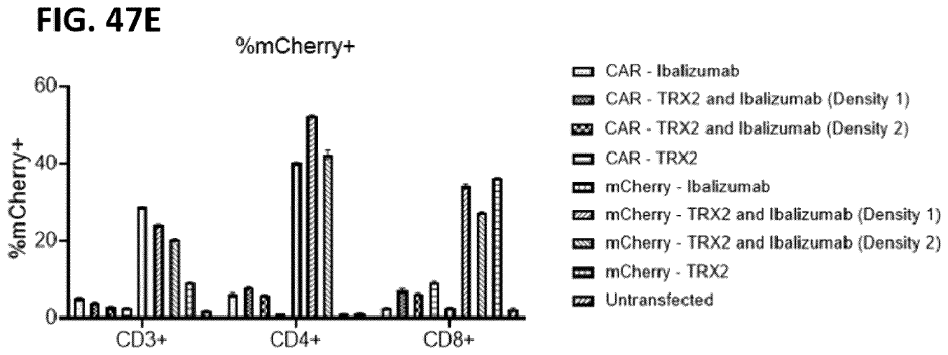

In some embodiments, the immune cell targeting group comprises an antibody selected from the group consisting of a Fab, F(ab′)₂, Fab′-SH, Fv, and scFv fragment. In some embodiments, the antibody is a human or humanized antibody. In some embodiments, the immune cell targeting group comprises a Fab or an immunoglobulin single variable domain, such as a Nanobody. In some embodiments, the immune cell targeting group comprises a Fab that does not comprise a natural interchain disulfide bond. For example, in some embodiments, the Fab comprises a heavy chain fragment that comprises a C₂₃₃S substitution, and/or a light chain fragment that comprises a C₂₁₄S substitution, numbering according to Kabat. In some embodiments, the immune cell targeting group comprises a Fab that comprises one or more non-native interchain disulfide bonds. In some embodiments, the interchain disulfide bonds are between two non-native cysteine residues on the light chain fragment and heavy chain fragment, respectively. For example, in some embodiments, the Fab comprises a heavy chain fragment that comprises F174C substitution, and/or a light chain fragment that comprises S176C substitution, numbering according to Kabat. In some embodiments, the Fab comprises a heavy chain fragment that comprises F174C and C₂₃₃S substitutions, and/or a light chain fragment that comprises S176C and C₂₁₄S substitutions, numbering according to Kabat. In some embodiments, the immune cell targeting group comprises a C-terminal cysteine residue. In some embodiments, the immune cell targeting group comprises a Fab that comprises a cysteine at the C-terminus of the heavy or light chain fragment. In some embodiments, the Fab further comprises one or more amino acids between the heavy chain of the Fab and the C-terminal cysteine. For example, in some embodiments, the Fab comprises two or more amino acids derived from an antibody hinge region (e.g., a partial hinge sequence) between the C-terminus of the Fab and the C-terminal cysteine. In some embodiments, the Fab comprises a heavy chain variable domain linked to an antibody CH1 domain and a light chain variable domain linked to an antibody light chain constant domain, wherein the CH1 domain and the light chain constant domain are linked by one or more interchain disulfide bonds, and wherein the immune cell targeting group further comprises a single chain variable fragment (scFv) linked to the C-terminus of the light chain constant domain by an amino acid linker. In some embodiments, the Fab antibody is a DS Fab, a NoDS Fab, a bDS Fab, a bDS Fab-ScFv, as demonstrated in FIG. 47.

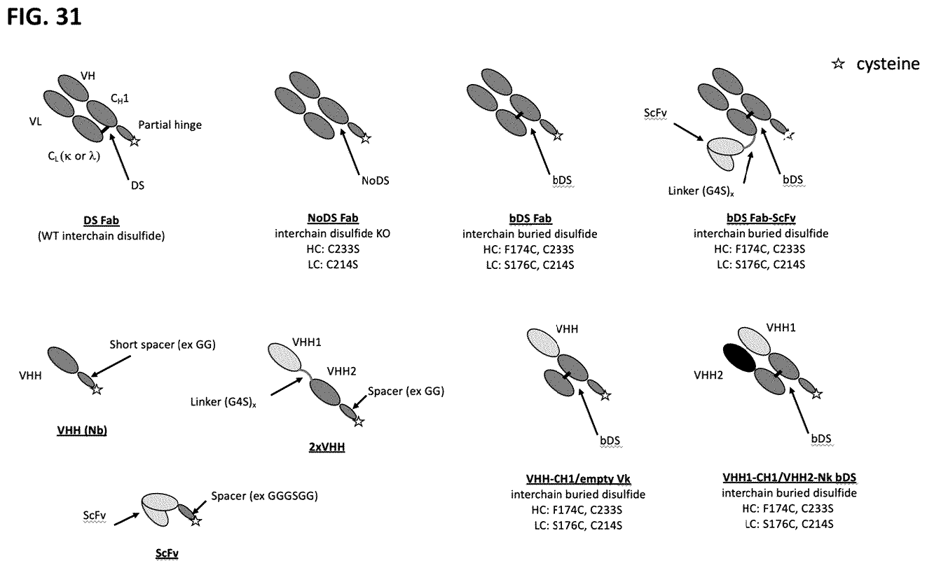

In some embodiments, the immune cell targeting group comprises an immunoglobulin single variable domain, such as a Nanobody (e.g., a V_{HH}). In some embodiments, the Nanobody comprises a cysteine at the C-terminus. In some embodiments, the Nanobody further comprises a spacer comprising one or more amino acids between the V_{HH} domain and the C-terminal cysteine. In some embodiments, the spacer comprises one or more glycine residues, e.g., two glycine residues. In some embodiments, the immune cell targeting group comprises two or more V_{HH} domains. In some embodiments, the two or more V_{HH} domains are linked by an amino acid linker. In some embodiments, the amino acid linker comprises one or more glycine and/or serine residues (e.g., one or more repeats of the sequence GGGGS). In some embodiments, the immune cell targeting group comprises a first V_{HH} domain linked to an antibody CH1 domain and a second V_{HH} domain linked to an antibody light chain constant domain, and wherein the antibody CH1 domain and the antibody light chain constant domain are linked by one or more disulfide bonds (e.g., interchain disulfide bonds). In some embodiments, the immune cell targeting group comprises a V_{HH} domain linked to an antibody CH1 domain, and wherein the antibody CH1 domain is linked to an antibody light chain constant domain by one or more disulfide bonds. In some embodiments, the CH1 domain comprises F174C and C₂₃₃S substitutions, and the light chain constant domain comprises S176C and C₂₁₄S substitutions, numbering according to Kabat. In some embodiments, the antibody is a ScFv, a V_{HH}, a 2×V_{HH}, a V_{HH}—CH1/empty Vk, or a V_{HH}1—CH₁/V_{HH-2}-Nb bDS, as demonstrated in FIG. 31.

An exemplary targeting moiety may have an amino sequence as set forth below:

Anti-CD3 hSP34-Fab sequences:
hSP34 heavy chain (HC) sequence (SEQ ID NO: 1):
EVQLVESGGGLVQPGGSLKLSCAASGFTFNKYAMNWVRQAPGKGLEWVARIRSKY
NNYATYYADSVKDRFTISRDDSKNTAYLQMNNLKTEDTAVYYCVRHGNFGNSYISY
WAYWGQGTLVTVSSASTKGPSVFPLAPSSKSTSGGTAALGCLVKDYFPEPVTVSWN
SGALTSGVHTFPAVLQSSGLYSLSSVVTVPSSSLGTQTYICNVNHKPSNTKVDKKVEP
KSSDKTHTC
hSP34-mlam light chain (LC) sequence (mouse lambda) (SEQ ID NO: 2):
QTVVTQEPSLTVSPGGTVTLTCGSSTGAVTSGNYPNWVQQKPGQAPRGLIGGTKFLA
PGTPARFSGSLLGGKAALTLSGVQPEDEAEYYCVLWYSNRWVFGGGTKLTVLGQPK
SSPSVTLFPPSSEELETNKATLVCTITDFYPGVVTVDWKVDGTPVTQGMETTQPSKQS
NNKYMASSYLTLTARAWERHSSYSCQVTHEGHTVEKSLSRADSS
SP34-hlam LC (human lambda) (SEQ ID NO: 3):
QTVVTQEPSLTVSPGGTVTLTCGSSTGAVTSGNYPNWVQQKPGQAPRGLIGGTKFLA
PGTPARFSGSLLGGKAALTLSGVQPEDEAEYYCVLWYSNRWVFGGGTKLTVLSQPK
AAPSVTLFPPSSEELQANKATLVCLVSDFYPGAVTVAWKADGSPVKVGVETTKPSK
QSNNKYAASSYLSLTPEQWKSHRSYSCRVTHEGSTVEKTVAPAESS
Anti-CD3 Hu291-Fab sequences:
Hu291 HC (SEQ ID NO: 4):
QVQLVQSGAEVKKPGASVKVSCKASGYTFISYTMHWVRQAPGQGLEWMGYINPRS
GYTHYNQKLKDKATLTADKSASTAYMELSSLRSEDTAVYYCARSAYYDYDGFAYW
GQGTLVTVSSASTKGPSVFPLAPSSKSTSGGTAALGCLVKDYFPEPVTVSWNSGALT
SGVHTFPAVLQSSGLYSLSSVVTVPSSSLGTQTYICNVNHKPSNTKVDKKVEPKSSDK
THTC
Hu 291 LC (SEQ ID NO: 5):
MDMRVPAQLLGLLLLWLPGAKCDIQMTQSPSSLSASVGDRVTITCSASSSVSYMNW
YQQKPGKAPKRLIYDTSKLASGVPSRFSGSGSGTDFTLTISSLQPEDFATYYCQQWSS
NPPTFGGGTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQGLSSPVTK
SFNRGES
Anti-CD8 TRX2-Fab sequences:
TRX2 HC (SEQ ID NO: 6):
QVQLVESGGGVVQPGRSLRLSCAASGFTFSDFGMNWVRQAPGKGLEWVALIYYDG
SNKFYADSVKGRFTISRDNSKNTLYLQMNSLRAEDTAVYYCAKPHYDGYYHFFDS
WGQGTLVTVSSASTKGPSVFPLAPSSKSTSGGTAALGCLVKDYFPEPVTVSWNSGAL
TSGVHTFPAVLQSSGLYSLSSVVTVPSSSLGTQTYICNVNHKPSNTKVDKKVEPKSSD
KTHTC
TRX2 LC (SEQ ID NO: 7):
DIQMTQSPSSLSASVGDRVTITCKGSQDINNYLAWYQQKPGKAPKLLIYNTDILHTG
VPSRFSGSGSGTDFTFTISSLQPEDIATYYCYQYNNGYTFGQGTKVEIKRTVAAPSVFI
FPPSDEQLKSGTASVVCLLNNFYPREAKVQWKVDNALQSGNSQESVTEQDSKDSTY
SLSSTLTLSKADYEKHKVYACEVTHQGLSSPVTKSFNRGES
Anti-CD8 OKT8-Fab sequences:
OKT8 HC (SEQ ID NO: 8):
QVQLVQSGAEDKKPGASVKVSCKASGFNIKDTYIHWVRQAPGQGLEWMGRIDPAN
DNTLYASKFQGRVTITADTSSNTAYMELSSLRSEDTAVYYCGRGYGYYVFDHWGQ
GTTVTVSSASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKKVEPKSSDKTH
TC
OKT8 LC (SEQ ID NO: 9):
DIVMTQSPSSLSASVGDRVTITCRTSRSISQYLAWYQEKPGKAPKLLIYSGSTLQSGVP
SRFSGSGSGTDFTLTISSLQPEDFATYYCQQHNENPLTFGQGTKVEIKRTVAAPSVFIF
PPSDEQLKSGTASVVCLLNNFYPREAKVQWKVDNALQSGNSQESVTEQDSKDSTYS
LSSTLTLSKADYEKHKVYACEVTHQGLSSPVTKSFNRGES
Anti-CD4 Ibalizumab-Fab sequences:
Ibalizumab HC (SEQ ID NO: 10):
QVQLQQSGPEVVKPGASVKMSCKASGYTFTSYVIHWVRQKPGQGLDWIGYINPYND
GTDYDEKFKGKATLTSDTSTSTAYMELSSLRSEDTAVYYCAREKDNYATGAWFAY
WGQGTLVTVSSASTKGPSVFPLAPSSKSTSGGTAALGCLVKDYFPEPVTVSWNSGAL
TSGVHTFPAVLQSSGLYSLSSVVTVPSSSLGTQTYICNVNHKPSNTKVDKKVEPKSSD
KTHTC
Ibalizumab LC (SEQ ID NO: 11):
DIVMTQSPDSLAVSLGERVTMNCKSSQSLLYSTNQKNYLAWYQQKPGQSPKLLIYW
ASTRESGVPDRFSGSGSGTDFTLTISSVQAEDVAVYYCQQYYSYRTFGGGTKLEIKRT
VAAPSVFIFPPSDEQLKSGTASVVCLLNNFYPREAKVQWKVDNALQSGNSQESVTEQ
DSKDSTYSLSSTLTLSKADYEKHKVYACEVTHQGLSSPVTKSFNRGES
anti-CD5 He3-Fab sequences:
He3 HC (SEQ ID NO: 12):
EIQLVQSGGGLVKPGGSVRISCAASGYTFTNYGMNWVRQAPGKGLEWMGWINTHT
GEPTYADSFKGRFTFSLDDSKNTAYLQINSLRAEDTAVYFCTRRGYDWYFDVWGQG
TTVTVSSASTKGPSVFPLAPSSKSTSGGTAALGCLVKDYFPEPVTVSWNSGALTSGVH
TFPAVLQSSGLYSLSSVVTVPSSSLGTQTYICNVNHKPSNTKVDKK VEPKSSDKTHTC
He3 LC (SEQ ID NO: 13):
DIQMTQSPSSLSASVGDRVTITCRASQDINSYLSWFQQKPGKAPKTLIYRANRLESGVP
SRFSGSGSGTDYTLTISSLQYEDFGIYYCQQYDESPWTFGGGTKLEIKRTVAAPSVFIFP
PSDEQLKSGTASVVCLLNNFYPREAKVQWKVDNALQSGNSQESVTEQDSKDSTYSLS
STLTLSKADYEKHKVYACEVTHQGLSSPVTKSFNRGES
anti-CD7 TH-69-Fab sequences:
TH-69 HC (SEQ ID NO: 14):
EVQLVESGGGLVKPGGSLKLSCAASGLTFSSYAMSWVRQTPEKRLEWVASISSGGFT
YYPDSVKGRFTISRDNARNILYLQMSSLRSEDTAMYYCARDEVRGYLDVWGAGTTV
TVSSASTKGPSVFPLAPSSKSTSGGTAALGCLVKDYFPEPVTVSWNSGALTSGVHTFP
AVLQSSGLYSLSSVVTVPSSSLGTQTYICNVNHKPSNTKVDKKVEPKSCDKTHTC
TH-69 LC (SEQ ID NO: 15):
DIQMTQTTSSLSASLGDRVTISCSASQGISNYLNWYQQKPDGTVKLLIYYTSSLHSGVP
SRFSGSGSGTDYSLTISNLEPEDIATYYCQQYSKLPYTFGGGTKLEIKRTVAAPSVFIFP
PSDEQLKSGTASVVCLLNNFYPREAKVQWKVDNALQSGNSQESVTEQDSKDSTYSLS
STLTLSKADYEKHKVYACEVTHQGLSSPVTKSFNRGEC
anti-CD2 TS2/18.1-Fab sequences:
TS2/18.1 HC (SEQ ID NO: 16):
EVQLVESGGGLVMPGGSLKLSCAASGFAFSSYDMSWVRQTPEKRLEWVAYISGGGF
TYYPDTVKGRFTLSRDNAKNTLYLQMSSLKSEDTAMYYCARQGANWELVYWGQGT
LVTVSAASTKGPSVFPLAPSSKSTSGGTAALGCLVKDYFPEPVTVSWNSGALTSGVHT
FPAVLQSSGLYSLSSVVTVPSSSLGTQTYICNVNHKPSNTKVDKKVEPKSSDKTHTC
TS2/18.1 LC (SEQ ID NO: 17):
DIVMTQSPATLSVTPGDRVFLSCRASQSISDFLHWYQQKSHESPRLLIKYASQSISGIPS
RFSGSGSGSDFTLSINSVEPEDVGVYFCQNGHNFPPTFGGGTKLEIKRTVAAPSVFIFPP
SDEQLKSGTASVVCLLNNFYPREAKVQWKVDNALQSGNSQESVTEQDSKDSTYSLSS
TLTLSKADYEKHKVYACEVTHQGLSSPVTKSFNRGES
anti-CD2 9.6-Fab sequences:
9.6 HC (SEQ ID NO: 18):
QVQLQQPGAELVRPGSSVKLSCKASGYTFTRYWIHWVKQRPIQGLEWIGNIDPSDSE
THYNQKFKDKATLTVDKSSGTAYMQLSSLTSEDSAVYYCATEDLYYAMEYWGQGT
SVTVSSASTKGPSVFPLAPSSKSTSGGTAALGCLVKDYFPEPVTVSWNSGALTSGVHT
FPAVLQSSGLYSLSSVVTVPSSSLGTQTYICNVNHKPSNTKVDKKVEPKSSDKTHTC
9.6 LC (SEQ ID NO: 19):
NIMMTQSPSSLAVSAGEKVTMTCKSSQSVLYSSNQKNYLAWYQQKPGQSPKLLIYW
ASTRESGVPDRFTGSGSGTDFTLTISSVQPEDLAVYYCHQYLSSHTFGGGTKLEIKRTV
AAPSVFIFPPSDEQLKSGTASVVCLLNNFYPREAKVQWKVDNALQSGNSQESVTEQD
SKDSTYSLSSTLTLSKADYEKHKVYACEVTHQGLSSPVTKSFNRGES
anti-CD2 9-1-Fab sequences:
9-1 HC (SEQ ID NO: 20):
QVQLQQPGTELVRPGSSVKLSCKASGYTFTSYWVNWVKQRPDQGLEWIGRIDPYDS
ETHYNQKFTDKAISTIDTSSNTAYMQLSTLTSDASAVYYCSRSPRDSSTNLADWGQG
TLVTVSSASTKGPSVFPLAPSSKSTSGGTAALGCLVKDYFPEPVTVSWNSGALTSGVH
TFPAVLQSSGLYSLSSVVTVPSSSLGTQTYICNVNHKPSNTKVDKKVEPKSSDKTHTC
9-1 LC (SEQ ID NO: 21):
DIVMTQSPATLSVTPGDRVSLSCRASQSISDYLHWYQQKSHESPRLLIKYASQSISGIPS
RFSGSGSGSDFTLSINSVEPEDVGVYYCQNGHSFPLTFGAGTKLELRRTVAAPSVFIFP
PSDEQLKSGTASVVCLLNNFYPREAKVQWKVDNALQSGNSQESVTEQDSKDSTYSLS
STLTLSKADYEKHKVYACEVTHQGLSSPVTKSFNRGES
mutOKT8-Fab sequences:
mutOKT8 HC (SEQ ID NO: 22):
QVQLVQSGAEDKKPGASVKVSCKASGFNIKDTYIHWVRQAPGQGLEWMGRIDPAND
NTLYASKFQGRVTITADTSSNTAYMELSSLRSEDTAVYYCGRGAGAYVFDHWGQGT
TVTVSSASTKGPSVFPLAPSSKSTSGGTAALGCLVKDYFPEPVTVSWNSGALTSGVHT
FPAVLQSSGLYSLSSVVTVPSSSLGTQTYICNVNHKPSNTKVDKKVEPKSSDKTHTC
mutOKT8 LC (SEQ ID NO: 23):
DIVMTQSPSSLSASVGDRVTITCRTSRSISAALAWYQEKPGKAPKLLIYSGSTLQSGVP
SRFSGSGSGTDFTLTISSLQPEDFATYYCQQHNENPLTFGQGTKVEIKRTVAAPSVFIFP
PSDEQLKSGTASVVCLLNNFYPREAKVQWKVDNALQSGNSQESVTEQDSKDSTYSLS
STLTLSKADYEKHKVYACEVTHQGLSSPVTKSFNRGES.
Anti-CD56 A1 Fab sequence
A1 bDS HC (SEQ ID NO: 26):
QVQLQQSGPGLVKPSQTLSLTCAISGDSVSSNSAAWNWIRQSPSNWIRQSPSGLEWL
GRTYYRSKWYNDYAVSVKSRITINPDTSKNQFSLQLNSVTPEDTAVYYCARENIAA
WTWAFDIWGQGTMVTVSSASTKGPSVFPLAPSSKSTSGGTAALGCLVKDYFPEPVT
VSWNSGALTSGVHTCPAVLQSSGLYSLSSVVTVPSSSLGTQTYICNVNHKPSNTKVD
KKVEPKSSDKTHTCGGHHHHHH
A1 bDS LC (SEQ ID NO: 27):
EIVMTQSPGTLSLSPGERATLSCRASQSVSSSYLAWYQQKPGLAPRLLIYDTSLRATDI
PDRFSGSGSGTAFTLTISRLEPEDFAVYYCQQYGSSPTFGQGTKVEIKRTVAAPSVFIF
PPSDEQLKSGTASVVCLLNNFYPREAKVQWKVDNALQSGNSQESVTEQDSKDSTYS
LCSTLTLSKADYEKHKVYACEVTHQGLSSPVTKSFNRGES
Anti-CD56 A2 Fab sequence
A2 bDS HC (SEQ ID NO: 28):
EVQLVQSGAEVKKPGSSVKVSCKASGGTFTGYYMHWVRQAPGQGLEWMGWINPN
SGGTNYAQKFQGRVTMTRDTSISTAYMELSRLRSDDTAVYYCARDLSSGYSGYFDY
WGQGTLVTVSSASTKGPSVFPLAPSSKSTSGGTAALGCLVKDYFPEPVTVSWNSGAL
TSGVHTCPAVLQSSGLYSLSSVVTVPSSSLGTQTYICNVNHKPSNTKVDKKVEPKSSD
KTHTCGGHHHHHH
A2 bDS LC (SEQ ID NO: 29):
DVVMTQSPLSLPVTPGEPASISCRSSQSLLHSNGYNYLNWYLQKPGQSPQLLIYLGSN
RASGVPDRFSGSGSGTDFTLKISRVEGEDVGDYYCMQALQSPFTFGQGTKLEIKRTV
AAPSVFIFPPSDEQLKSGTASVVCLLNNFYPREAKVQWKVDNALQSGNSQESVTEQD
SKDSTYSLCSTLTLSKADYEKHKVYACEVTHQGLSSPVTKSFNRGES
Anti-CD56 A3 Fab sequence
A3 bDS HC (SEQ ID NO: 30):
EVQLVQSGAEVKKPGSSVKVSCKASGGTFTGYYMHWVRQAPGQGLEWMGWINPN
SGGTNYAQKFQGRVTMTRDTSISTAYMELSRLRSDDTAVYYCARDLSSGYSGYFDY
WGQGTLVTVSSASTKGPSVFPLAPSSKSTSGGTAALGCLVKDYFPEPVTVSWNSGAL
TSGVHTCPAVLQSSGLYSLSSVVTVPSSSLGTQTYICNVNHKPSNTKVDKKVEPKSSD
KTHTCGGHHHHHH
A3 bDS LC (SEQ ID NO: 31):
DVVMTQSPLSLPVTPGEPASISCRSSQSLLHSNGYNFLDWYLQKPGQSPQLLIYLGSN
RASGVPDRFSGSGSGTDFTLKISRVEADDVGVYYCMQSLQTPWTFGHGTKVEIKRTV
AAPSVFIFPPSDEQLKSGTASVVCLLNNFYPREAKVQWKVDNALQSGNSQESVTEQD
SKDSTYSLCSTLTLSKADYEKHKVYACEVTHQGLSSPVTKSFNRGES
Anti-CD56 Lorvotuzumab Fab sequence
Lorvotuzumab bDS HC (SEQ ID NO: 32):
QVQL VESGGG VVQPGRSLRL SCAASGFTFS SFGMHWVRQA
PGKGLEWVAYISSGSFTIYY ADSVKGRFTI SRDNSKNTLY LQMNSLRAED
TAVYYCARMR KGYAMDYWGQ
GTLVTVSSASTKGPSVFPLAPSSKSTSGGTAALGCLVKDYFPEPVTVSWNSGALTSG
VHTCPAVLQSSGLYSLSSVVTVPSSSLGTQTYICNVNHKPSNTKVDKKVEPKSSDKT
HTCHHHHHH
Lorvotuzumab bDS LC (SEQ ID NO: 33):
DVVMTQSPLSLPVTLGQPASISCRSSQIIIHSDGNTYLEWFQQRPGQSPRRLIYKVSNR
FSGVPDRFSGSGSGTDFTLKISRVEAEDVGVYYCFQGSHVPHTFGQGTKVEIKRTVA
APSVFIFPPSDEQLKSGTASVVCLLNNFYPREAKVQWKVDNALQSGNSQESVTEQDS
KDSTYSLCSTLTLSKADYEKHKVYACEVTHQGLSSPVTKSFNRGES
Anti-CD2 RPA-2.10v1 Fab sequence
RPA-2.10v1 bDS HC (SEQ ID NO: 34):
EVKLVESGGGLVKPGGSLKLSCAASGFTFSSYDMSWVRQTPEKRLEWVASISGGGFL
YYLDSVKGRFTISRDNARNILYLHMTSLRSEDTAMYYCARSSYGEIMDYWGQGTSV
TVSSASTKGPSVFPLAPSSKSTSGGTAALGCLVKDYFPEPVTVSWNSGALTSGVHTCP
AVLQSSGLYSLSSVVTVPSSSLGTQTYICNVNHKPSNTKVDKKVEPKSSDKTHTCHH
HHHH
RPA-2.10v1 bDS LC (SEQ ID NO: 35):
DILLTQSPAILSVSPGER VSFSCRASQRIGTSIHWYQQRTTGSPRLLIKYASESISGIPSR
FSGSGSGTDFTLSINSVESEDVADYYCQQSHGWPFTFGGGTKLEIERTVAAPSVFIFPP
SDEQLKSGTASVVCLLNNFYPREAKVQWKVDNALQSGNSQESVTEQDSKDSTYSLC
STLTLSKADYEKHKVYACEVTHQGLSSPVTKSFNRGES
Anti-CD137 4B4-1 Fab sequence
4B4-1 bDS HC (SEQ ID NO: 36):
QVQLQQPGAELVKPGASVKLSCKASGYTFSSYWMHWVKQRPGQVLEWIGEINPGN
GHTNYNEKFKSKATLTVDKSSSTAYMQLSSLTSEDSAVYYCARSFTTARGFAYWGQ
GTLVTVSASTKGPSVFPLAPSSKSTSGGTAALGCLVKDYFPEPVTVSWNSGALTSGV
HTCPAVLQSSGLYSLSSVVTVPSSSLGTQTYICNVNHKPSNTKVDKKVEPKSSDKTHT
CHHHHHH
4B4-1 bDS LC (SEQ ID NO: 37):
DIVMTQSPATQSVTPGDRVSLSCRASQTISDYLHWYQQKSHESPRLLIKYASQSISGIP
SRFSGSGSGSDFTLSINSVEPEDVGVYYCQDGHSFPPTFGGGTKLEIKRTVAAPSVFIF
PPSDEQLKSGTASVVCLLNNFYPREAKVQWKVDNALQSGNSQESVTEQDSKDSTYS
LCSTLTLSKADYEKHKVYACEVTHQGLSSPVTKSFNRGES
hSP34-hlam NoDS HC (SEQ ID NO: 38):
EVQLVESGGGLVQPGGSLKLSCAASGFTFNKYAMNWVRQAPGKGLEWVARIRSKY
NNYATYYADSVKDRFTISRDDSKNTAYLQMNNLKTEDTAVYYCVRHGNFGNSYISY
WAYWGQGTLVTVSSASTKGPSVFPLAPSSKSTSGGTAALGCLVKDYFPEPVTVSWN
SGALTSGVHTFPAVLQSSGLYSLSSVVTVPSSSLGTQTYICNVNHKPSNTKVDKKVEP
KSSDKTHTC
hSP34-hlam NoDS LC (SEQ ID NO: 39):
QTVVTQEPSLTVSPGGTVTLTCGSSTGAVTSGNYPNWVQQKPGQAPRGLIGGTKFLA
PGTPARFSGSLLGGKAALTLSGVQPEDEAEYYCVLWYSNRWVFGGGTKLTVLSQPK
AAPSVTLFPPSSEELQANKATLVCLVSDFYPGAVTVAWKADGSPVKVGVETTKPSK
QSNNKYAASSYLSLTPEQWKSHRSYSCRVTHEGSTVEKTVAPAESS
hSP34-hlam DS HC (SEQ ID NO: 40):
EVQLVESGGGLVQPGGSLKLSCAASGFTFNKYAMNWVRQAPGKGLEWVARIRSKY
NNYATYYADSVKDRFTISRDDSKNTAYLQMNNLKTEDTAVYYCVRHGNFGNSYISY
WAYWGQGTLVTVSSASTKGPSVFPLAPSSKSTSGGTAALGCLVKDYFPEPVTVSWN
SGALTSGVHTFPAVLQSSGLYSLSSVVTVPSSSLGTQTYICNVNHKPSNTKVDKKVEP
KSCDKTHTC
hSP34-hlam DS LC (SEQ ID NO: 41):
QTVVTQEPSLTVSPGGTVTLTCGSSTGAVTSGNYPNWVQQKPGQAPRGLIGGTKFLA
PGTPARFSGSLLGGKAALTLSGVQPEDEAEYYCVLWYSNRWVFGGGTKLTVLSQPK
AAPSVTLFPPSSEELQANKATLVCLVSDFYPGAVTVAWKADGSPVKVGVETTKPSK
QSNNKYAASSYLSLTPEQWKSHRSYSCRVTHEGSTVEKTVAPAECS
Anti-CD2 TS2/18.1 DS Fab
TS2/18.1 DS HC (SEQ ID NO: 42):
EVQLVESGGGLVMPGGSLKLSCAASGFAFSSYDMSWVRQTPEKRLEWVAYISGGGF
TYYPDTVKGRFTLSRDNAKNTLYLQMSSLKSEDTAMYYCARQGANWELVYWGQG
TLVTVSAASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEPKSCDKTHTC
TS2/18.1 DS LC (SEQ ID NO: 43):
DIVMTQSPATLSVTPGDRVFLSCRASQSISDFLHWYQQKSHESPRLLIKYASQSISGIPS
RFSGSGSGSDFTLSINSVEPEDVGVYFCQNGHNFPPTFGGGTKLEIKRTVAAPSVFIFP
PSDEQLKSGTASVVCLLNNFYPREAKVQWKVDNALQSGNSQESVTEQDSKDSTYSL
SSTLTLSKADYEKHKVYACEVTHQGLSSPVTKSFNRGEC
Anti-CD2 9.6 DS Fab
9.6 DS HC (SEQ ID NO: 44):
QVQLQQPGAELVRPGSSVKLSCKASGYTFTRYWIHWVKQRPIQGLEWIGNIDPSDSE
THYNQKFKDKATLTVDKSSGTAYMQLSSLTSEDSAVYYCATEDLYYAMEYWGQGT
SVTVSSASTKGPSVFPLAPSSKSTSGGTAALGCLVKDYFPEPVTVSWNSGALTSGVHT
FPAVLQSSGLYSLSSVVTVPSSSLGTQTYICNVNHKPSNTKVDKKVEPKSCDKTHTC
9.6 DS LC (SEQ ID NO: 45):
NIMMTQSPSSLAVSAGEKVTMTCKSSQSVLYSSNQKNYLAWYQQKPGQSPKLLIYW
ASTRESGVPDRFTGSGSGTDFTLTISSVQPEDLAVYYCHQYLSSHTFGGGTKLEIKRTV
AAPSVFIFPPSDEQLKSGTASVVCLLNNFYPREAKVQWKVDNALQSGNSQESVTEQD
SKDSTYSLSSTLTLSKADYEKHKVYACEVTHQGLSSPVTKSFNRGEC
hSP34-hlam bDS HC (SEQ ID NO: 46):
EVQLVESGGGLVQPGGSLKLSCAASGFTFNKYAMNWVRQAPGKGLEWVARIRSKY
NNYATYYADSVKDRFTISRDDSKNTAYLQMNNLKTEDTAVYYCVRHGNFGNSYISY
WAYWGQGTLVTVSSASTKGPSVFPLAPSSKSTSGGTAALGCLVKDYFPEPVTVSWN
SGALTSGVHTCPAVLQSSGLYSLSSVVTVPSSSLGTQTYICNVNHKPSNTKVDKKVEP
KSSDKTHTCHHHHHH
hSP34-hlam bDS LC (SEQ ID NO: 47):
QTVVTQEPSLTVSPGGTVTLTCGSSTGAVTSGNYPNWVQQKPGQAPRGLIGGTKFLA
PGTPARFSGSLLGGKAALTLSGVQPEDEAEYYCVLWYSNRWVFGGGTKLTVLSQPK
AAPSVTLFPPSSEELQANKATLVCLVSDFYPGAVTVAWKADGSPVKVGVETTKPSK
QSNNKYAACSYLSLTPEQWKSHRSYSCRVTHEGSTVEKTVAPAESS
Anti-CD3 TR66 bDS Fab sequence
TR66 bDS HC (SEQ ID NO: 48):
QVQLQQSGAELARPGASVKMSCKTSGYTFTRYTMHWVKQRPGQGLEWIGYINPSR
GYTNYNQKFKDKATLTTDKSSSTAYMQLSSLTSEDSAVYYCARYYDDNYSLDYWG
QGTTLTVSSASTKGPSVFPLAPSSKSTSGGTAALGCLVKDYFPEPVTVSWNSGALTSG
VHTCPAVLQSSGLYSLSSVVTVPSSSLGTQTYICNVNHKPSNTKVDKKVEPKSSDKT
HTCHHHHHH
TR66 bDS LC (SEQ ID NO: 49):
QIVLTQSPSSLSASLGEKVTMTCRASSSVSYMNWYQQKPGTSPKRWIYDTSKVASGV
PDRFSGSGSGTSYSLTISSMEAEDAATYYCQQWSSNPLTFGAGTKLELKRTVAAPSV
FIFPPSDEQLKSGTASVVCLLNNFYPREAKVQWKVDNALQSGNSQESVTEQDSKDST
YSLCSTLTLSKADYEKHKVYACEVTHQGLSSPVTKSFNRGES
Anti-CD3 TRX4 bDS Fab sequence
TRX4 bDS HC (SEQ ID NO: 50):
EVQLLESGGGLVQPGGSLRLSCAASGFTFSSFPMAWVRQAPGKGLEWVSTISTSGGR
TYYRDSVKGRFTISRDNSKNTLYLQMNSLRAEDTAVYYCAKFRQYSGGFDYWGQG
TLVTVSSASTKGPSVFPLAPSSKSTSGGTAALGCLVKDYFPEPVTVSWNSGALTSGV
HTCPAVLQSSGLYSLSSVVTVPSSSLGTQTYICNVNHKPSNTKVDKKVEPKSSDKTHT
CHHHHHH
TRX4 bDS LC (SEQ ID NO: 51):
DIQLTQPNSVSTSLGSTVKLSCTLSSGNIENNYVHWYQLYEGRSPTTMIYDDDKRPD
GVPDRFSGSIDRSSNSAFLTIHNVAIEDEAIYFCHSYVSSFNVFGGGTKLTVLGQPKAN
PTVTLFPPSSEELQANKATLVCLISDFYPGAVTVAWKADGSPVKAGVETTKPSKQSN
NKYAACSYLSLTPEQWKSHRSYSCQVTHEGSTVEKTVAPTESS
Anti-CD3 HzUCHT1 bDS Fab sequence
HzUCHT1(Y59T) bDS HC (SEQ ID NO: 52):
EVQLVESGGGLVQPGGSLRLSCAASGYSFTGYTMNWVRQAPGKGLEWVALINPTK
GVSTYNQKFKDRFTISVDKSKNTAYLQMNSLRAEDTAVYYCARSGYYGDSDWYFD
VWGQGTLVTVSSASTKGPSVFPLAPSSKSTSGGTAALGCLVKDYFPEPVTVSWNSGA
LTSGVHTCPAVLQSSGLYSLSSVVTVPSSSLGTQTYICNVNHKPSNTKVDKKVEPKSS
DKTHTCHHHHHH
HzUCHT1 bDS LC (SEQ ID NO: 53):
DIQMTQSPSSLSASVGDRVTITCRASQDIRNYLNWYQQKPGKAPKLLIYYTSRLESGV
PSRFSGSGSGTDYTLTISSLQPEDFATYYCQQGNTLPWTFGQGTKVEIKRTVAAPSVFI
FPPSDEQLKSGTASVVCLLNNFYPREAKVQWKVDNALQSGNSQESVTEQDSKDSTY
SLCSTLTLSKADYEKHKVYACEVTHQGLSSPVTKSFNRGES
Anti-CD3 Teplizumab bDS Fab sequence
Teplizumab bDS HC (SEQ ID NO: 54):
QVQLVQSGGGVVQPGRSLRLSCKASGYTFTRYTMHWVRQAPGKGLEWIGYINPSRG
YTNYNQKVKDRFTISRDNSKNTAFLQMDSLRPEDTGVYFCARYYDDHYCLDYWGQ
GTPVTVSSASTKGPSVFPLAPSSKSTSGGTAALGCLVKDYFPEPVTVSWNSGALTSGV
HTCPAVLQSSGLYSLSSVVTVPSSSLGTQTYICNVNHKPSNTKVDKKVEPKSSDKTHT
CHHHHHH
Teplizumab bDS LC (SEQ ID NO: 55):
DIQMTQSPSSLSASVGDRVTITCSASSSVSYMNWYQQTPGKAPKRWIYDTSKLASGV
PSRFSGSGSGTDYTFTISSLQPEDIATYYCQQWSSNPFTFGQGTKLQITRTVAAPSVFIF
PPSDEQLKSGTASVVCLLNNFYPREAKVQWKVDNALQSGNSQESVTEQDSKDSTYS
LCSTLTLSKADYEKHKVYACEVTHQGLSSPVTKSFNRGES
Anti-CD8 TRX2 bDS Fab sequence
TRX2 bDS HC (SEQ ID NO: 56):
QVQLVESGGGVVQPGRSLRLSCAASGFTFSDFGMNWVRQAPGKGLEWVALIYYDG
SNKFYADSVKGRFTISRDNSKNTLYLQMNSLRAEDTAVYYCAKPHYDGYYHFFDS
WGQGTLVTVSSASTKGPSVFPLAPSSKSTSGGTAALGCLVKDYFPEPVTVSWNSGAL
TSGVHTCPAVLQSSGLYSLSSVVTVPSSSLGTQTYICNVNHKPSNTKVDKKVEPKSSD
KTHTC
TRX2 bDS LC (SEQ ID NO: 57):
DIQMTQSPSSLSASVGDRVTITCKGSQDINNYLAWYQQKPGKAPKLLIYNTDILHTG
VPSRFSGSGSGTDFTFTISSLQPEDIATYYCYQYNNGYTFGQGTKVEIKRTVAAPSVFI
FPPSDEQLKSGTASVVCLLNNFYPREAKVQWKVDNALQSGNSQESVTEQDSKDSTY
SLCSTLTLSKADYEKHKVYACEVTHQGLSSPVTKSFNRGES
Anti-CD2 Lo-CD2b bDS Fab sequence
Lo-CD2b bDS HC (SEQ ID NO: 58):
EVQLVESGGGLVQPGASLKLSCVASGFTFSDYWMSWVRQTPGKPMEWIGHIKYDGS
YTNYAPSLKNRFTISRDNAKTTLYLQMSNVRSEDSATYYCAREAPGAASYWGQGTL
VTVSSASTKGPSVFPLAPSSKSTSGGTAALGCLVKDYFPEPVTVSWNSGALTSGVHT
CPAVLQSSGLYSLSSVVTVPSSSLGTQTYICNVNHKPSNTKVDKKVEPKSSDKTHTC
Lo-CD2b bDS LC (SEQ ID NO: 59):
DVVLTQTPVAQPVTLGDQASISCRSSQSLVHSNGNTYLEWFLQKPGQSPQLLIYKVS
NRFSGVPDRFIGSGSGSDFTLKISRVEPEDWGVYYCFQGTHDPYTFGAGTKLELKRT
VAAPSVFIFPPSDEQLKSGTASVVCLLNNFYPREAKVQWKVDNALQSGNSQESVTEQ
DSKDSTYSLCSTLTLSKADYEKHKVYACEVTHQGLSSPVTKSFNRGES
Anti-CD2 35.1 bDS Fab sequence
35.1 bDS HC (SEQ ID NO: 60):
EVQLQQSGAELVKPGASVKLSCRTSGFNIKDTYIHWVKQRPEQGLKWIGRIDPANGN
TKYDPKFQDKATVTADTSSNTAYLQLSSLTSEDTAVYYCVTYAYDGNWYFDVWGA
GTAVTVSSASTKGPSVFPLAPSSKSTSGGTAALGCLVKDYFPEPVTVSWNSGALTSG
VHTCPAVLQSSGLYSLSSVVTVPSSSLGTQTYICNVNHKPSNTKVDKKVEPKSSDKT
HTC
35.1 bDS LC (SEQ ID NO: 61):
DIKMTQSPSSMYVSLGERVTITCKASQDINSFLSWFQQKPGKSPKTLIYRANRLVDGV
PSRFSGSGSGQDYSLTISSLEYEDMEIYYCLQYDEFPYTFGGGTKLEMKRTVAAPSVF
IFPPSDEQLKSGTASVVCLLNNFYPREAKVQWKVDNALQSGNSQESVTEQDSKDSTY
SLCSTLTLSKADYEKHKVYACEVTHQGLSSPVTKSFNRGES
Anti-CD2 OKT11 bDS Fab sequence
OKT11 bDS HC (SEQ ID NO: 62):
QVQLQQPGAELVRPGTSVKLSCKASGYTFTSYWMHWIKQRPEQGLEWIGRIDPYDS
ETHYNEKFKDKAILSVDKSSSTAYIQLSSLTSDDSAVYYCSRRDAKYDGYALDYWG
QGTSVTVSSASTKGPSVFPLAPSSKSTSGGTAALGCLVKDYFPEPVTVSWNSGALTSG
VHTCPAVLQSSGLYSLSSVVTVPSSSLGTQTYICNVNHKPSNTKVDKKVEPKSSDKT
HTC
OKT11 bDS LC (SEQ ID NO: 63):
DIVMTQAAPSVPVTPGESVSISCRSSKTLLHSNGNTYLYWFLQRPGQSPQVLIYRMSN
LASGVPNRFSGSGSETTFTLRISRVEAEDVGIYYCMQHLEYPYTFGGGTKLEIERTVA
APSVFIFPPSDEQLKSGTASVVCLLNNFYPREAKVQWKVDNALQSGNSQESVTEQDS
KDSTYSLCSTLTLSKADYEKHKVYACEVTHQGLSSPVTKSFNRGES
Anti-CD11a HzMHM24 bDS Fab sequence
HzMHM24 bDS HC (SEQ ID NO: 64):
EVQLVESGGGLVQPGGSLRLSCAASGYSFTGHWMNWVRQAPGKGLEWVGMIHPSD
SETRYNQKFKDRFTISVDKSKNTLYLQMNSLRAEDTAVYYCARGIYFYGTTYFDYW
GQGTLVTVSSASTKGPSVFPLAPSSKSTSGGTAALGCLVKDYFPEPVTVSWNSGALT
SGVHTCPAVLQSSGLYSLSSVVTVPSSSLGTQTYICNVNHKPSNTKVDKKVEPKSSD
KTHTCHHHHHH
HzMHM24 bDS LC (SEQ ID NO: 65):
DIQMTQSPSSLSASVGDRVTITCRASKTISKYLAWYQQKPGKAPKLLIYSGSTLQSGV
PSRFSGSGSGTDFTLTISSLQPEDFATYYCQQHNEYPLTFGQGTKVEIKRTVAAPSVFI
FPPSDEQLKSGTASVVCLLNNFYPREAKVQWKVDNALQSGNSQESVTEQDSKDSTY
SLCSTLTLSKADYEKHKVYACEVTHQGLSSPVTKSFNRGES
Anti-CD18 h1B4 bDS Fab sequence
h1B4 bDS HC (SEQ ID NO: 66):
EVQLVESGGDLVQPGRSLRLSCAASGFTFSDYYMSWVRQAPGKGLEWVAAIDNDG
GSISYPDTVKGRFTISRDNAKNSLYLQMNSLRVEDTALYYCARQGRLRRDYFDYWG
QGTLVTVSTASTKGPSVFPLAPSSKSTSGGTAALGCLVKDYFPEPVTVSWNSGALTS
GVHTCPAVLQSSGLYSLSSVVTVPSSSLGTQTYICNVNHKPSNTKVDKKVEPKSSDK
THTCHHHHHH
h1B4 bDS LC (SEQ ID NO: 67):
DIQMTQSPSSLSASVGDRVTITCRASESVDSYGNSFMHWYQQKPGKAPKLLIYRASN
LESGVPSRFSGSGSGTDFTFTISSLQPEDIATYYCQQSNEDPLTFGQGTKLEIKRTVAA
PSVFIFPPSDEQLKSGTASVVCLLNNFYPREAKVQWKVDNALQSGNSQESVTEQDSK
DSTYSLCSTLTLSKADYEKHKVYACEVTHQGLSSPVTKSFNRGES
Anti-CD18 Erlizumab bDS Fab sequence
Erlizumab bDS HC (SEQ ID NO: 68):
EVQLVESGGGLVQPGGSLRLSCATSGYTFTEYTMHWMRQAPGKGLEWVAGINPKN
GGTSHNQRFMDRFTISVDKSTSTAYMQMNSLRAEDTAVYYCARWRGLNYGFDVRY
FDVWGQGTLVTVSSASTKGPSVFPLAPSSKSTSGGTAALGCLVKDYFPEPVTVSWNS
GALTSGVHTCPAVLQSSGLYSLSSVVTVPSSSLGTQTYICNVNHKPSNTKVDKKVEP
KSSDKTHTCHHHHHH
Erlizumab bDS LC (SEQ ID NO: 69):
DIQMTQSPSSLSASVGDRVTITCRASQDINNYLNWYQQKPGKAPKLLIYYTSTLHSG
VPSRFSGSGSGTDYTLTISSLQPEDFATYYCQQGNTLPPTFGQGTKVEIKRTVAAPSVF
IFPPSDEQLKSGTASVVCLLNNFYPREAKVQWKVDNALQSGNSQESVTEQDSKDSTY
SLCSTLTLSKADYEKHKVYACEVTHQGLSSPVTKSFNRGES
Anti-CD4/CD8 Ibalizumab/TRX2 bDS Fab-ScFv sequence
Ibalizumab/TRX2 bDS Fab-ScFv HC (SEQ ID NO: 70):
QVQLQQSGPEVVKPGASVKMSCKASGYTFTSYVIHWVRQKPGQGLDWIGYINPYND
GTDYDEKFKGKATLTSDTSTSTAYMELSSLRSEDTAVYYCAREKDNYATGAWFAY
WGQGTLVTVSSASTKGPSVFPLAPSSKSTSGGTAALGCLVKDYFPEPVTVSWNSGAL
TSGVHTCPAVLQSSGLYSLSSVVTVPSSSLGTQTYICNVNHKPSNTKVDKKVEPKSSD
KTHTCHHHHHH
Ibalizumab/TRX2 bDS Fab-ScFv LC (SEQ ID NO: 71):
DIVMTQSPDSLAVSLGERVTMNCKSSQSLLYSTNQKNYLAWYQQKPGQSPKLLIYW
ASTRESGVPDRFSGSGSGTDFTLTISSVQAEDVAVYYCQQYYSYRTFGGGTKLEIKRT
VAAPSVFIFPPSDEQLKSGTASVVCLLNNFYPREAKVQWKVDNALQSGNSQESVTEQ
DSKDSTYSLCSTLTLSKADYEKHKVYACEVTHQGLSSPVTKSFNRGESGGGGSGGG
GSGGGGSQVQLVESGGGVVQPGRSLRLSCAASGFTFSDFGMNWVRQAPGKGLEWV
ALIYYDGSNKFYADSVKGRFTISRDNSKNTLYLQMNSLRAEDTAVYYCAKPHYDGY
YHFFDSWGQGTLVTVSSGGGGSGGGGSGGGGSGGGGSDIQMTQSPSSLSASVGDRV
TITCKGSQDINNYLAWYQQKPGKAPKLLIYNTDILHTGVPSRFSGSGSGTDFTFTISSL
QPEDIATYYCYQYNNGYTFGQGTKVEIK
Anti-CD4 Ibalizumab NoDS Fab sequence
Ibalizumab NoDS LC (SEQ ID NO: 72):
QVQLQQSGPEVVKPGASVKMSCKASGYTFTSYVIHWVRQKPGQGLDWIGYINPYND
GTDYDEKFKGKATLTSDTSTSTAYMELSSLRSEDTAVYYCAREKDNYATGAWFAY
WGQGTLVTVSSASTKGPSVFPLAPSSKSTSGGTAALGCLVKDYFPEPVTVSWNSGAL
TSGVHTFPAVLQSSGLYSLSSVVTVPSSSLGTQTYICNVNHKPSNTKVDKKVEPKSSD
KTHTC
Ibalizumab NoDS HC (SEQ ID NO: 73):
DIVMTQSPDSLAVSLGERVTMNCKSSQSLLYSTNQKNYLAWYQQKPGQSPKLLIYW
ASTRESGVPDRFSGSGSGTDFTLTISSVQAEDVAVYYCQQYYSYRTFGGGTKLEIKRT
VAAPSVFIFPPSDEQLKSGTASVVCLLNNFYPREAKVQWKVDNALQSGNSQESVTEQ
DSKDSTYSLSSTLTLSKADYEKHKVYACEVTHQGLSSPVTKSFNRGES
Anti-CD4 OKT4 bDS Fab sequence
OKT4 bDS LC (SEQ ID NO: 74):
EVQLVESGGGLVQPGGSLRLSCAASGFTFSNYAMSWVRQAPGKRLEWVSAISDHST
NTYYPDSVKGRFTISRDNAKNTLYLQMNSLRAEDTAVYYCARKYGGDYDPFDYWG
QGTLVTVSSASTKGPSVFPLAPSSKSTSGGTAALGCLVKDYFPEPVTVSWNSGALTS
GVHTCPAVLQSSGLYSLSSVVTVPSSSLGTQTYICNVNHKPSNTKVDKKVEPKSSDK
THTCHHHHHH
OKT4 bDS HC (SEQ ID NO: 75):
DIQMTQSPSSLSASVGDRVTITCQASQDINNYIAWYQHKPGKGPKLLIHYTSTLQPGIP
SRFSGSGSGRDYTLTISSLQPEDFATYYCLQYDNLLFTFGGGTKVEIKRTVAAPSVFIF
PPSDEQLKSGTASVVCLLNNFYPREAKVQWKVDNALQSGNSQESVTEQDSKDSTYS
LCSTLTLSKADYEKHKVYACEVTHQGLSSPVTKSFNRGES
Anti-CD4 T023200008 Nb sequence (SEQ ID NO: 76)
CDR1, CDR2, CDR3 underlined based on IMGT designation:
EVQLVESGGGSVQPGGSLTLSCGTSGRTFNVMGWFRQAPGKEREFVAAVRWSSTGI
YYTQYADSVKSRFTISRDNAKNTVYLEMNSLKPEDTAVYYCAADTYNSNPARWDG
YDFRGQGTLVTVSSGGCGGHHHHHH
Anti-CD8 BDSn Nb sequence (SEQ ID NO: 77)
CDR1, CDR2, CDR3 underlined based on IMGT designation:
EVQLVESGGGLVQAGGSLRLSCAASGSTFSDYGVGWFRQAPGKGREFVADIDWNGE
HTSYADSVKGRFATSRDNAKNTAYLQMNSLKPEDTAVYYCAADALPYTVRKYNYW
GQGTQVTVSSGGCGGHHHHHH
Anti-CD3 T0170117G03-A Nb sequence (SEQ ID NO: 78)
EVQLVESGGGPVQAGGSLRLSCAASGRTYRGYSMGWFRQAPGKEREFVAAIVWSG
GNTYYEDSVKGRFTISRDNAKNIMYLQMTSLKPEDSATYYCAAKIRPYIFKIAGQYD
YWGQGTLVTVSSAGGGSGGHHHHHHC
Anti-CD3 T0170060E11 Nb sequence (SEQ ID NO: 79)
EVQLVESGGGLVQPGGSLRLSCAASGDIYKSFDMGWYRQAPGKQRDLVAVIGSRGN
NRGRTNYADSVKGRFTISRDGTGNTVYLLMNKLRPEDTAIYYCNTAPLVAGRPWGR
GTLVTVSSGGGSGGHHHHHHC
Anti-CD7 V1 Nb sequence (SEQ ID NO: 80)
DVQLQESGGGLVQAGGSLRLSCAVSGYPYSSYCMGWFRQAPGKEREGVAAIDSDG
RTRYADSVKGRFTISQDNAKNTLYLQMNRMKPEDTAMYYCAARFGPMGCVDLSTL
SFGHWGQGTQVTVSITGGGCHHHHHHHH
Anti-TCR T017000700 Nb sequence (SEQ ID NO: 81)
CDR1, CDR2, CDR3 underlined based on IMGT designation:
EVQLVESGGGVVQPGGSLRLSCVASGYVHKINFYGWYRQAPGKEREKVAHISIGDQ
TDYADSAKGRFTISRDESKNTVYLQMNSLRPEDTAAYYCRALSRIWPYDYWGQGTL
VTVSSGGCGGHHHHHH
Anti-CD28 28CD065G01 Nb sequence (SEQ ID NO: 82)
EVQLVESGGGLVQPGGSLRLSCAASGSIFRLHTMEWYRRTPETQREWVATITSGGTT
NYPDSVKGRFTISRDDTKKTVYLQMNSLKPEDTAVYYCHAVATEDAGFPPSNYWG
QGTLVTVSSGGCGGHHHHHH
Anti-CD3 T0170061C09 Nb sequence (SEQ ID NO: 83)
EVQLVESGGGPVQAGGSLRLSCAASGRTYRGYSMGWFRQAPGREREFVAAIVWSD
GNTYYEDSVKGRFTISRDNAKNTMYLQMTSLKPEDSATYYCAAKIRPYIFKIAGQYD
YWGQGTLVTVSSGGCGGHHHHHH
Anti-CD3 12D2 bDS Fab sequence
12D2 bDS HC (SEQ ID NO: 84):
EVKLVESGGGLVQPGRSLRLSCAASGFNFYAYWMGWVRQAPGKGLEWIGEIKKDG
TTINYTPSLKDRFTISRDNAQNTLYLQMTKLGSEDTALYYCAREERDGYFDYWGQG
VMVTVSSASTKGPSVFPLAPSSKSTSGGTAALGCLVKDYFPEPVTVSWNSGALTSGV
HTCPAVLQSSGLYSLSSVVTVPSSSLGTQTYICNVNHKPSNTKVDKKVEPKSSDKTHT
CGGHHHHHH
12D2 bDS LC (SEQ ID NO: 85):
QFVLTQPNSVSTNLGSTVKLSCKRSTGNIGSNYVNWYQQHEGRSPTTMIYRDDKRPD
GVPDRFSGSIDRSSNSALLTINNVQTEDEADYFCQSYSSGIVFGGGTKLTVLSQPKAA
PSVTLFPPSSEELQANKATLVCLVSDFYPGAVTVAWKADGSPVKVGVETTKPSKQSN
NKYAACSYLSLTPEQWKSHRSYSCRVTHEGSTVEKTVAPAESS
Anti-CD28 8G8A Fab sequence
8G8A bDS HC (SEQ ID NO: 86):
EVQLQQSGPELVKPGASVKMSCKASGYTFTSYVIQWVKQKPGQGLEWIGSINPYND
YTKYNEKFKGKATLTSDKSSITAYMEFSLTSEDSALYCARWGDGNYWGRGTLTVSS
ASTKGPSVFPLAPSSKSTSGGTAALGCLVKDYFPEPVTVSWNSGALTSGVHTCPAVL
QSSGLYSLSSVVTVPSSSLGTQTYICNVNHKPSNTKVDKKVEPKSSDKTHTCGGHHH
HHH
8G8A bDS LC (SEQ ID NO: 87):
DIEMTQSPAIMSASLGER VTMTCTASSSVSSSYFHWYQKPGSSPKLCIYSTSNLASGV
PPRFSGSGSTSYSLTISMEAEDAATYFCHQYHRSPTFGGGTKLETKRTVAAPSVFIFPP
SDEQLKSGTASVVCLLNNFYPREAKVQWKVDNALQSGNSQESVTEQDSKDSTYSLC
STLTLSKADYEKHKVYACEVTHQGLSSPVTKSFNRGES
Anti-CD28 2E12 Fab sequence
2E12 bDS HC (SEQ ID NO: 88):
QVQLKESGPGLVAPSQSLSITCTVSGFSLTGYGVNWVRQPPGKGLEWLGMIWGDGS
TDYNSALKSRLSITKDNSKSQVFLKMNSLQTDDTARYYCARDGYSNFHYYVMDYW
GQGTSVTVSSASTKGPSVFPLAPSSKSTSGGTAALGCLVKDYFPEPVTVSWNSGALTS
GVHTCPAVLQSSGLYSLSSVVTVPSSSLGTQTYICNVNHKPSNTKVDKKVEPKSSDK
THTCGGHHHHHH
2E12 bDS LC (SEQ ID NO: 89):
DIVLTQSPASLAVSLGQRATISCRASESVEYYVTSLMQWYQQKPGQPPKLLISAASNV
ESGVPARFSGSGSGTDFSLNIHPVEEDDIAMYFCQQSRKVPWTFGGGTKLEIKRRTVA
APSVFIFPPSDEQLKSGTASVVCLLNNFYPREAKVQWKVDNALQSGNSQESVTEQDS
KDSTYSLCSTLTLSKADYEKHKVYACEVTHQGLSSPVTKSFNRGES
Anti-CD28 CD28.9.3 Fab sequence
CD28.9.3 bDS HC (SEQ ID NO: 90):
QVKLQQSGPGLVTPSQSLSITCTVSGFSLSDYGVHWVRQSPGQGLEWLGVIWAGGG
TNYNSALMSRKSISKDNSKSQVFLKMNSLQADDTAVYYCARDKGYSYYYSMDYW
GQGTTVTVSSASTKGPSVFPLAPSSKSTSGGTAALGCLVKDYFPEPVTVSWNSGALT
SGVHTCPAVLQSSGLYSLSSVVTVPSSSLGTQTYICNVNHKPSNTKVDKKVEPKSSD
KTHTCGGHHHHHH
CD28.9.3 bDS LC (SEQ ID NO: 91):
DIVLTQSPAS LAVSLGQRAT ISCRASESVEYYVTSLMQWY QQKPGQPPKL
LIFAASNVES GVPARFSGSG SGTNFSLNIHPVDEDDVAMY FCQQSRKVPY
TFGGGTKLEIKRTVAAPSVFIFPPSDEQLKSGTASVVCLLNNFYPREAKVQWKVDNA
LQSGNSQESVTEQDSKDSTYSLCSTLTLSKADYEKHKVYACEVTHQGLSSPVTKSFN
RGES
Anti-CD28 HzTN228 Fab sequence
HzTN228 bDS HC (SEQ ID NO: 92):
QVQLQESGPGLVKPSETLSLTCAVSGFSLTSYGVHWIRQPGKGLEWLGVIWPGTNFN
SALMSRLTISEDTSKNQVSLKLSSVTAADTAVYCARDRAYGNYLYAMDYWGQGTL
VTVSSASTKGPSVFPLAPSSKSTSGGTAALGCLVKDYFPEPVTVSWNSGALTSGVHT
CPAVLQSSGLYSLSSVVTVPSSSLGTQTYICNVNHKPSNTKVDKKVEPKSSDKTHTC
GGHHHHHH
HzTN228 bDS LC (SEQ ID NO: 93):
DIQMTQSPSLSASVGDRVTITCRASESVEYVTSLMQWYQKPGKAPKLLIYAASNVDS
GVPSRFSGSGTDFTLTISLQPEDIATYCQSRKVPFTFGGGTKVEIKRTVAAPSVFIFPPS
DEQLKSGTASVVCLLNNFYPREAKVQWKVDNALQSGNSQESVTEQDSKDSTYSLCS
TLTLSKADYEKHKVYACEVTHQGLSSPVTKSFNRGES
Anti-CD28 TGN2122.C Fab sequence
TGN2122.C bDS HC (SEQ ID NO: 94):
QVQLVQSGAEVKKPGASVKVSCKASGYTFTDYKIHWVRQAPGQGLEWIGYIYPYSG
SSDYNQKFKSRATLTVDNSISTAYMELSRLRSDDTAVYYCARGGDAMDYWGQGTL
VTVSSASTKGPSVFPLAPSSKSTSGGTAALGCLVKDYFPEPVTVSWNSGALTSGVHT
CPAVLQSSGLYSLSSVVTVPSSSLGTQTYICNVNHKPSNTKVDKKVEPKSSDKTHTC
GGHHHHHH
TGN2122.C bDS LC (SEQ ID NO: 95):
DIQMTQSPSSLSASVGDRVTITCGASENIYGALNWYQRKPGKAPKLLIYGATNLADG
VPSRFSGSGSGRDYTLTISSLQPEDFATYFCQNILGTWTFGGGTKVEIKRTVAAPSVFI
FPPSDEQLKSGTASVVCLLNNFYPREAKVQWKVDNALQSGNSQESVTEQDSKDSTY
SLCSTLTLSKADYEKHKVYACEVTHQGLSSPVTKSFNRGES
Anti-CD28 TGN2122.H Fab sequence
TGN2122.H bDS HC (SEQ ID NO: 96):
EVQLVESGGGLVQPGGSLRLSCAASGFTFNIYYMSWVRQAPGKGLELVAAINPDGG
NTYYPDTVKGRFTISRDNAKNSLYLQMNSLRAEDTAVYYCARYGGPGFDSWGQGT
LVTVSSASTKGPSVFPLAPSSKSTSGGTAALGCLVKDYFPEPVTVSWNSGALTSGVH
TCPAVLQSSGLYSLSSVVTVPSSSLGTQTYICNVNHKPSNTKVDKKVEPKSSDKTHTC
GGHHHHHH
TGN2122.H bDS LC (SEQ ID NO: 97):
ENVLTQSPATLSLSPGERATLSCSASSSVSYMHWYQQKPGQAPRLWIYDTSKLASGIP
ARFSGSGSRNDYTLTISSLEPEDFAVYYCFPGSGFPFMYTFGGGTKVEIKRTVAAPSV
FIFPPSDEQLKSGTASVVCLLNNFYPREAKVQWKVDNALQSGNSQESVTEQDSKDST
YSLCSTLTLSKADYEKHKVYACEVTHQGLSSPVTKSFNRGES
Anti-CD8 TRX2 ScFv sequence (SEQ ID NO: 98):
QVQL VESGGGVVQPGRSLRLSCAASGFTFSDFGMNWVRQAPGKGLEWVALIYYDG
SNKFYADSVKGRFTISRDNSKNTLYLQMNSLRAEDTAVYYCAKPHYDGYYHFFDS
WGQGTLVTVSSGGGGSGGGGSGGGGSGGGGSDIQMTQSPSSLSASVGDRVTITCKG
SQDINNYLAWYQQKPGKAPKLLIYNTDILHTGVPSRFSGSGSGTDFTFTISSLQPEDIA
TYYCYQYNNGYTFGQGTKVEIKGGGSGGCGGHHHHHH
V1 VHH-CH1 bDS HC (SEQ ID NO: 99):
DVQLQESGGGLVQAGGSLRLSCAVSGYPYSSYCMGWFRQAPGKEREGVAAIDSDG
RTRYADSVKGRFTISQDNAKNTLYLQMNRMKPEDTAMYYCAARFGPMGCVDLSTL
SFGHWGQGTQVTVSITASTKGPSVFPLAPSSKSTSGGTAALGCLVKDYFPEPVTVSW
NSGALTSGVHTCPAVLQSSGLYSLSSVVTVPSSSLGTQTYICNVNHKPSNTKVDKKV
EPKSSDKTHTCGGHHHHHH

In some embodiments, the targeting moiety comprises a polypeptide sequence as disclosed herein. In some embodiments, the targeting moiety comprises all six CDRs of a polypeptide sequence as disclosed herein. In some embodiments, the targeting moiety comprises CDR1, CDR2, and CDR3 of an immunoglobulin single variable domain (ISVD) as disclosed herein. In further embodiments, the targeting moiety binds to the same epitope on the targeting molecule that a polypeptide sequence as disclosed herein binds to. In further embodiments, the targeting moiety competes with a polypeptide sequence as disclosed herein to bind to the same epitope on the targeting molecule.

In certain embodiments, the targeting group or immune cell targeting group (e.g., T cell-targeting agent, B cell-targeting agent, or NK-cell targeting agent) may be covalently coupled to a lipid via a polyethylene glycol (PEG) containing linker.

In other embodiments, the lipid used to create a conjugate may be selected from

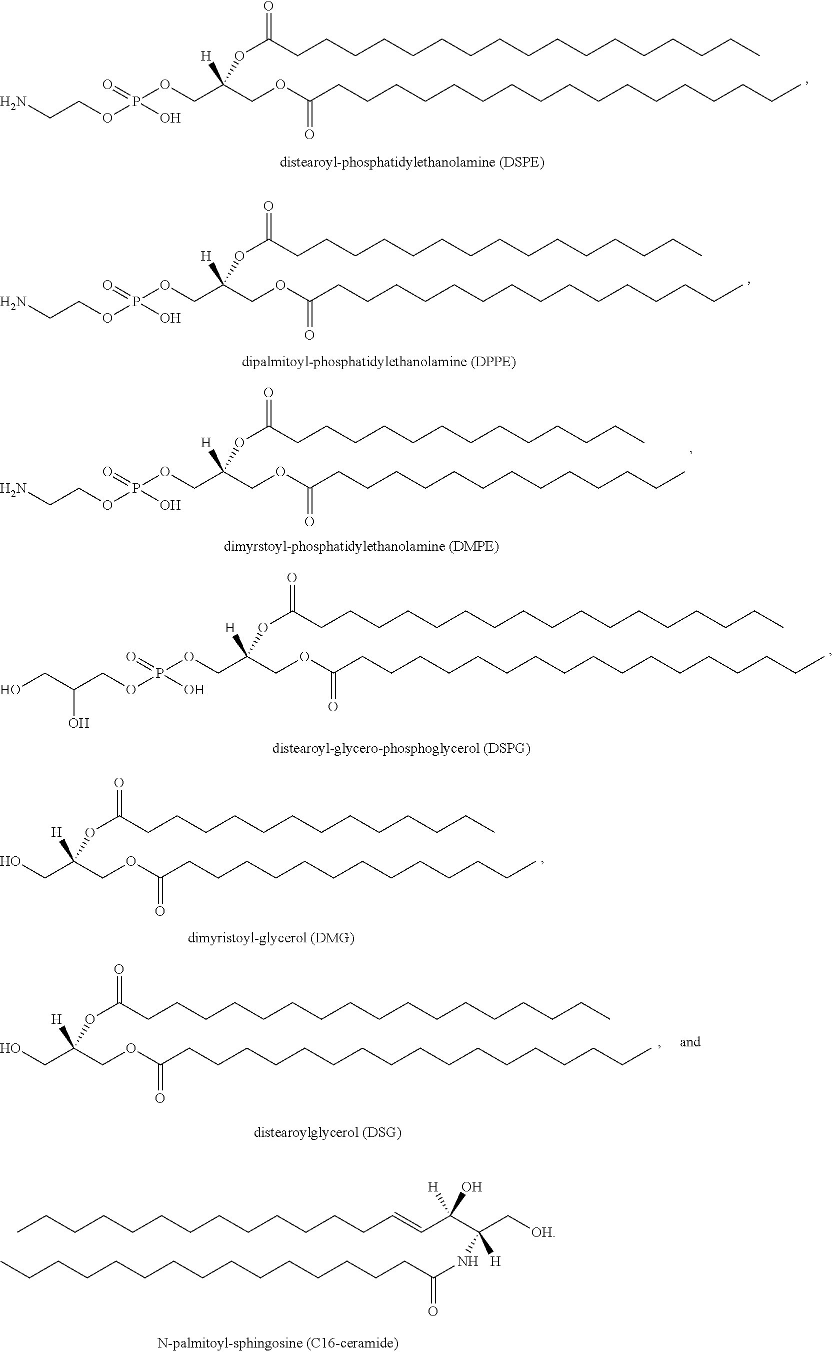

The immune cell targeting group can be covalently coupled to a lipid either directly or via a linker, for example, a polyethylene glycol (PEG) containing linker. In certain embodiments, the PEG is PEG 1000, PEG 2000, PEG 3400, PEG 3000, PEG 3450, PEG 4000, or PEG 5000. In certain, embodiments, the PEG is PEG 2000.

In some embodiments, the lipid-immune cell targeting group conjugate is present in the lipid blend in a range of 0.001-0.5 mole percent, 0.001-0.3 mole percent, 0.002-0.2 mole percent, 0.01-0.1 mole percent, 0.1-0.3 mole percent, or 0.1-0.2 mole percent.

In certain embodiments, the lipid immune-cell targeting agent conjugate comprises DSPE, a PEG component and a targeting antibody. In certain embodiments, the antibody is a T-cell targeting agent, for example, an anti-CD2 antibody, an anti-CD3 antibody, an anti-CD4 antibody, an anti-CD5 antibody, an anti-CD7 antibody, an anti CD8 antibody, or an anti-TCR β antibody.

An exemplary lipid-immune cell targeting group conjugate comprises DSPE and PEG 2000, for example, as described in Nellis et al. (2005) BIOTECHNOL. PROG. 21, 205-220. An exemplary conjugate comprises the structure of Formula (III), where the scFv represents an engineered antibody binding site that binds to a target of interest. In certain embodiments, the engineered antibody binding site binds to any of the targets described hereinabove. In certain embodiments, the engineered antibody binding site can be, for example, an engineered anti-CD3 antibody or an engineered anti-CD8 antibody. In certain embodiments, the engineered antibody binding site can be, for example, an engineered anti-CD2 antibody or an engineered anti-CD7 antibody.

An example of a compound of Formula (III) is as shown below:

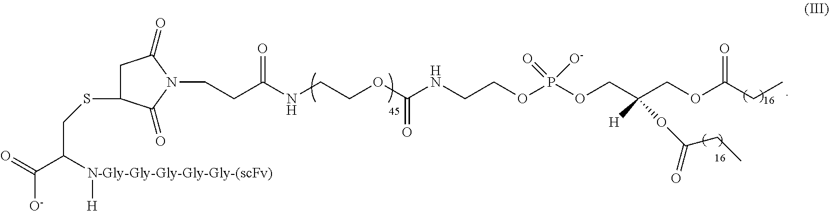

It is contemplated that the scFv in Formula (III) may be replaced with an intact antibody or an antigen fragment thereof (e.g., a Fab).

Another example of a compound of Formula (IV) is as shown below:

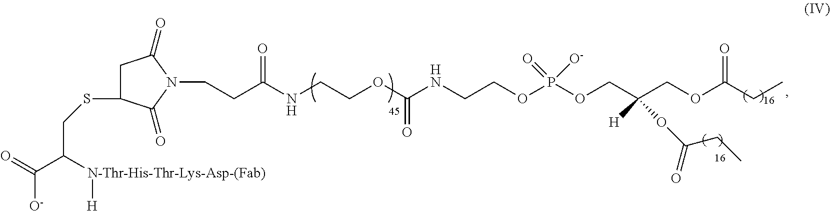

the production of which is described in Nellis et al. (2005) supra, or U.S. Pat. No. 7,022,336. It is contemplated that the Fab in Formula (IV) may be replaced with an intact antibody or an antigen fragment thereof (e.g., an (Fab′)₂ fragment) or an engineering antibody binding site (e.g., an scFv).

Other lipid immune cell target group conjugates are described, for example, in U.S. Pat. No. 7,022,336, where the targeting group may be replaced with a targeting group of interest, for example, a targeting group that binds an T-cell or NK cell surface antigen as described hereinabove.

In certain embodiments, the lipid component of an exemplary conjugate of Formula (II) can be any of the lipids described herein. In some embodiments, the lipid component of a conjugate of Formula (II) is based on an ionizable, cationic lipid described herein, for example, an ionizable, cationic lipid of Formula (I), Formula (Ia), Formula (Ib), or a slat thereof. For example, an exemplary ionizable, cationic lipid can be selected from Table 1, or a salt thereof.

In certain embodiments, the conjugate based on a lipid of the present disclosure may include:

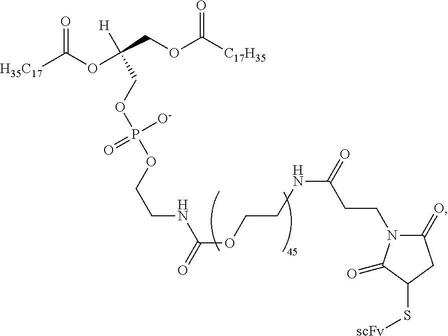

where scFv represents an engineered antibody binding site that binds a target described hereinabove, e.g., CD2, CD3, CD7, or CD8.
In certain embodiments, the lipid blend may further comprise free PEG-lipid so as to reduce the amount of non-specific binding via the targeting group. The free PEG-lipid can be the same or different from the PEG-lipid included in the conjugate. In certain embodiments, the free PEG-lipid is selected from the group consisting of PEG-distearoyl-phosphatidylethanolamine (PEG-DSPE) or PEG-dimyrstoyl-phosphatidylethanolamine (PEG-DMPE), N-(Methylpolyoxyethylene oxycarbonyl)-1,2-dipalmitoyl-sn-glycero-3-phosphoethanolamine (DPPE-PEG) 1,2-Dimyristoyl-rac-glycero-3-methylpolyoxyethylene (PEG-DMG), 1,2-Dipalmitoyl-rac-glycero-3-methylpolyoxyethylene (PEG-DPG), 1,2-Dioleoyl-rac-glycerol, methoxypolyethylene Glycol (DOG-PEG) 1,2-Distearoyl-rac-glycero-3-methylpolyoxyethylene (PEG-DSG), N-palmitoyl-sphingosine-1-{succinyl[methoxy(polyethylene glycol)] (PEG-ceramide), DSPE-PEG-cysteine, or a derivative thereof, all with average PEG lengths between 2000-5000, with 2000, 3400, or 5000. A final composition may contain a mixture of two or more of these pegylated lipids. In certain embodiments, the LNP composition comprises a mixture of PEG-lipids with myristoyl and stearic acyl chains. In certain embodiments, the LNP composition comprises a mixture of PEG-lipids with palmitoyl and stearoyl acyl chains.

In certain embodiments, the derivative of the PEG-lipid has a methyoxy, hydroxyl or a carboxylic acid end group at the PEG terminus.

The lipid-immune cell targeting group conjugate can be incorporated into LNPs as described below, for example, in LNPs containing, for example, an ionizable cationic lipid, a sterol, a neutral phospholipid and a PEG-lipid. It is contemplated that, in certain embodiments, the LNPs containing the lipid-immune cell targeting group can contain an ionizable cationic lipid described herein or a cationic lipid described, for example, in U.S. Pat. Nos. 10,221,127, 10,653,780 or U.S. Published application No. US2018/0085474, US2016/0317676, International Publication No. WO2009/086558, or Miao et al. (2019) NATURE BIOTECH 37:1174-1185, or Jayaraman et al. (2012) ANGEW CHEM INT. 51: 8529-8533.

In some embodiments, the cationic lipid can be selected from an ionizable cationic lipid set forth in Table 1, or a salt thereof.

| | |
|---|---|
| 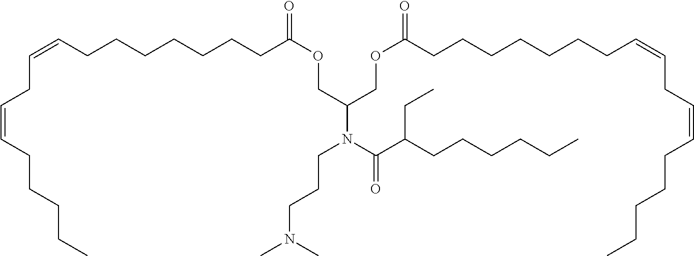 [SMILES: CCCCC/C=C\C/C=C\CCCCCCCC(=O)OCC(COC(=O)CCCCCCC/C=C\C/C=C\CCCCC)N(CCCN(C)C)C(=O)C(CC)CCCCCC] | Lipid 1 |
| 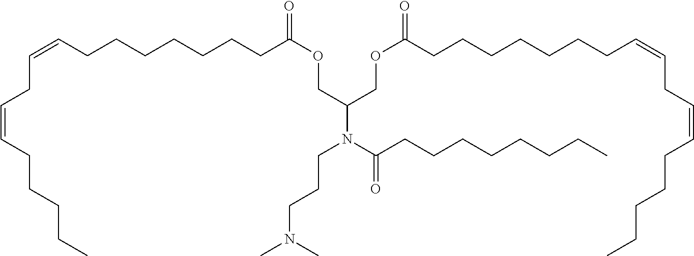 [SMILES: CCCCC/C=C\C/C=C\CCCCCCCC(=O)OCC(COC(=O)CCCCCCC/C=C\C/C=C\CCCCC)N(CCCN(C)C)C(=O)CCCCCCCC] | Lipid 2 |
| 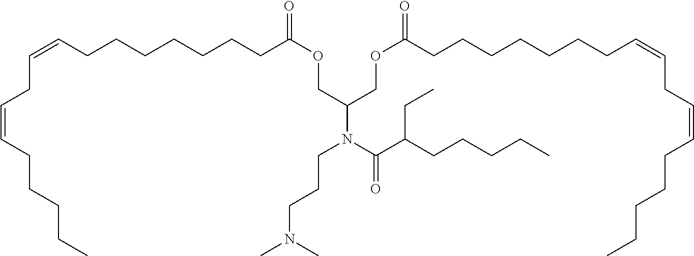 [SMILES: CCCCC/C=C\C/C=C\CCCCCCCC(=O)OCC(COC(=O)CCCCCCC/C=C\C/C=C\CCCCC)N(CCCN(C)C)C(=O)C(CC)CCCCC] | Lipid 3 |
| 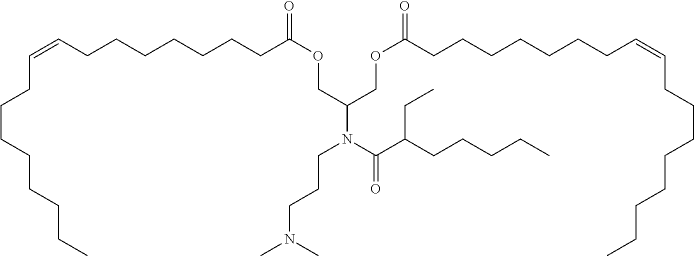 [SMILES: CCCCCCCC/C=C\CCCCCCCC(=O)OCC(COC(=O)CCCCCCC/C=C\CCCCCCCC)N(CCCN(C)C)C(=O)C(CC)CCCCC] | Lipid 4 |
| 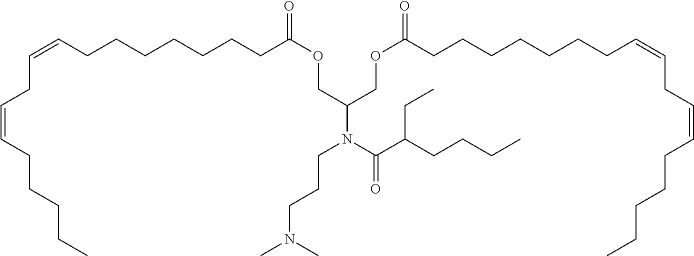 [SMILES: CCCCC/C=C\C/C=C\CCCCCCCC(=O)OCC(COC(=O)CCCCCCC/C=C\C/C=C\CCCCC)N(CCCN(C)C)C(=O)C(CC)CCCC] | Lipid 5 |
| 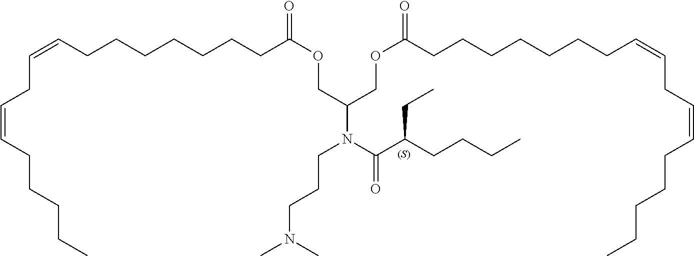 | Lipid 5A |
| 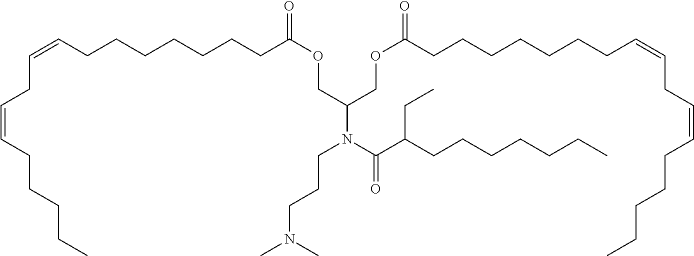 [SMILES: CCCCC/C=C\C/C=C\CCCCCCCC(=O)OCC(COC(=O)CCCCCCC/C=C\C/C=C\CCCCC)N(CCCN(C)C)C(=O)C(CC)CCCCCCC] | Lipid 6 |
| 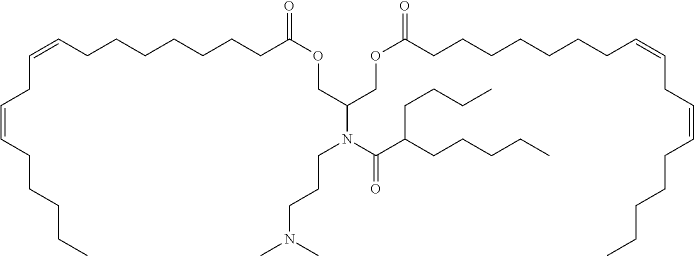 [SMILES: CCCCC/C=C\C/C=C\CCCCCCCC(=O)OCC(COC(=O)CCCCCCC/C=C\C/C=C\CCCCC)N(CCCN(C)C)C(=O)C(CCCC)CCCCC] | Lipid 7 |
| 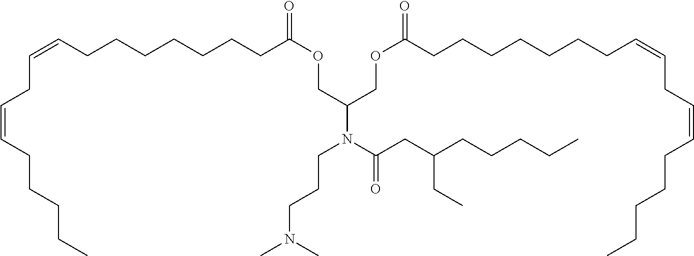 [SMILES: CCCCC/C=C\C/C=C\CCCCCCCC(=O)OCC(COC(=O)CCCCCCC/C=C\C/C=C\CCCCC)N(CCCN(C)C)C(=O)CC(CC)CCCCC] | Lipid 8 |
| 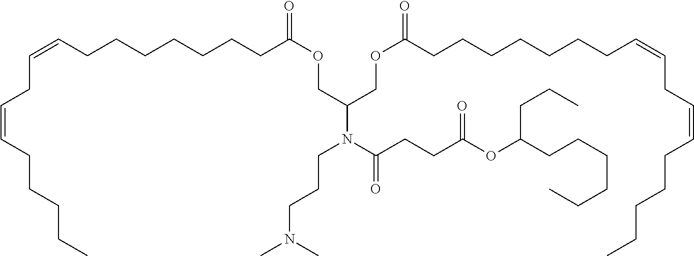 [SMILES: CCCCC/C=C\C/C=C\CCCCCCCC(=O)OCC(COC(=O)CCCCCCC/C=C\C/C=C\CCCCC)N(CCCN(C)C)C(=O)CCC(=O)OC(CCC)CCCCCC] | Lipid 9 |
| 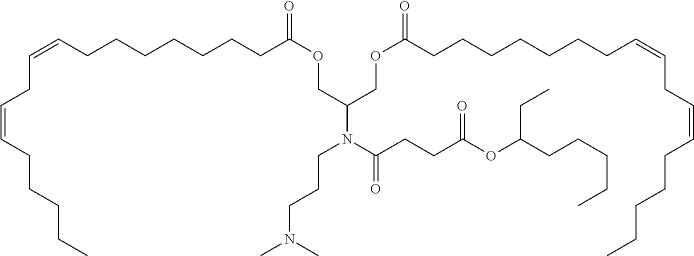 [SMILES: CCCCC/C=C\C/C=C\CCCCCCCC(=O)OCC(COC(=O)CCCCCCC/C=C\C/C=C\CCCCC)N(CCCN(C)C)C(=O)CCC(=O)OC(CC)CCCCC] | Lipid 10 |
| 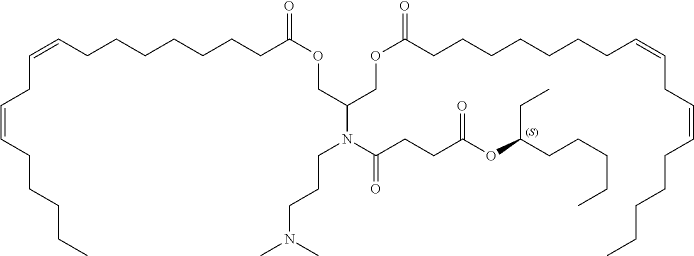 | Lipid 10A |
| 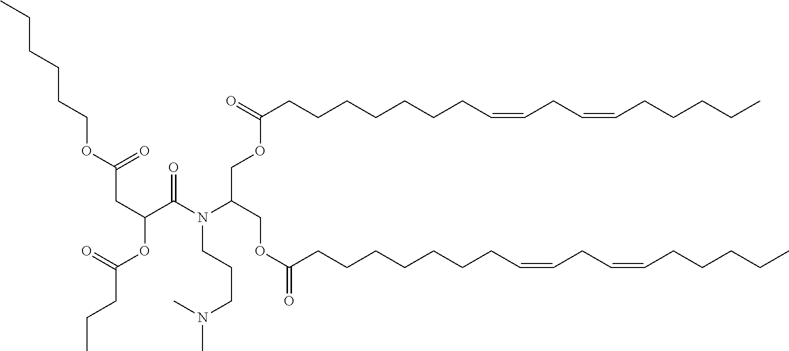 [SMILES: CCCCC/C=C\C/C=C\CCCCCCCC(=O)OCC(COC(=O)CCCCCCC/C=C\C/C=C\CCCCC)N(CCCN(C)C)C(=O)C(CC(=O)OCCCCCC)OC(=O)CCC] | Lipid 11 |
| 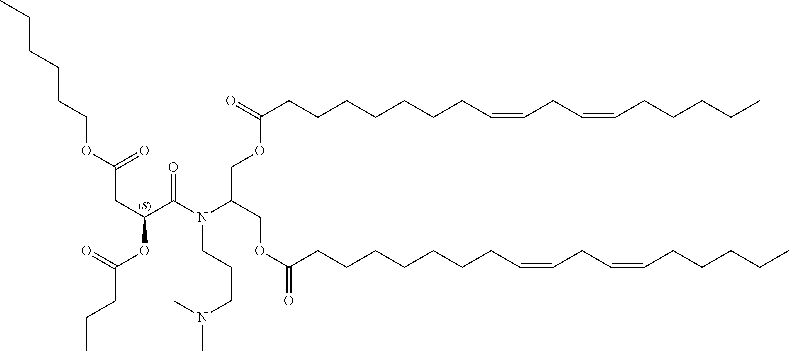 | Lipid 11A |
| 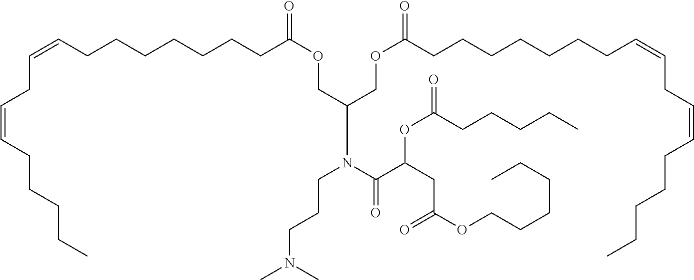 [SMILES: CCCCC/C=C\C/C=C\CCCCCCCC(=O)OCC(COC(=O)CCCCCCC/C=C\C/C=C\CCCCC)N(CCCN(C)C)C(=O)C(CC(=O)OCCCCCC)OC(=O)CCCCC] | Lipid 12 |
| 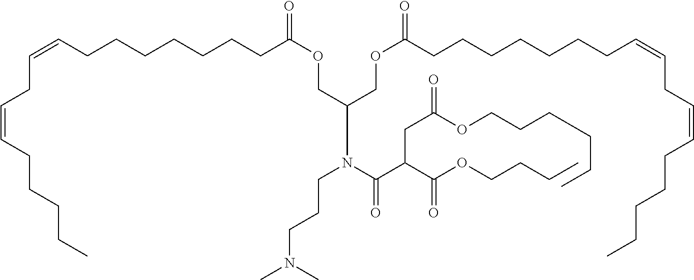 [SMILES: CCCCC/C=C\C/C=C\CCCCCCCC(=O)OCC(COC(=O)CCCCCCC/C=C\C/C=C\CCCCC)N(CCCN(C)C)C(=O)C(CC(=O)OCCCCCC)C(=O)OCCCC] | Lipid 13 |
| 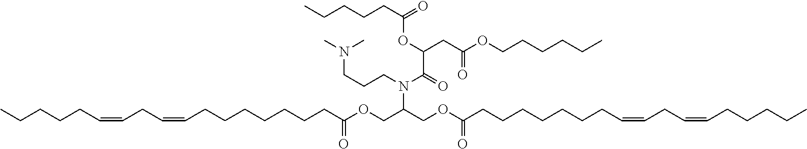 [SMILES: CCCCC/C=C\C/C=C\CCCCCCCC(=O)OCC(COC(=O)CCCCCCC/C=C\C/C=C\CCCCC)N(CCCN(C)C)C(=O)C(CC(=O)OCCCCCC)OC(=O)CCCCC] | Lipid 14 |
| 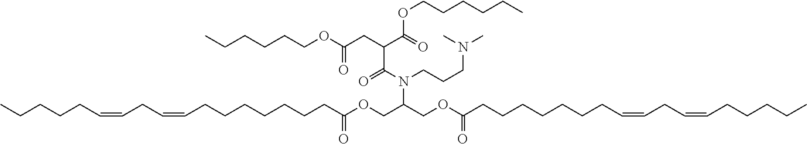 [SMILES: CCCCC/C=C\C/C=C\CCCCCCCC(=O)OCC(COC(=O)CCCCCCC/C=C\C/C=C\CCCCC)N(CCCN(C)C)C(=O)C(CC(=O)OCCCCCC)C(=O)OCCCCCC] | Lipid 14A |
| 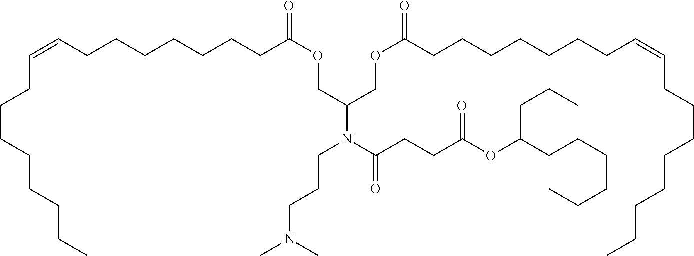 [SMILES: CCCCCCCC/C=C\CCCCCCCC(=O)OCC(COC(=O)CCCCCCC/C=C\CCCCCCCC)N(CCCN(C)C)C(=O)CCC(=O)OC(CCC)CCCCCC] | Lipid 15 |
| 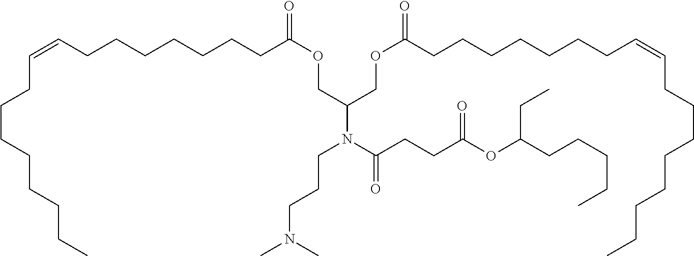 [SMILES: CCCCCCCC/C=C\CCCCCCCC(=O)OCC(COC(=O)CCCCCCC/C=C\CCCCCCCC)N(CCCN(C)C)C(=O)CCC(=O)OC(CC)CCCCC] | Lipid 16 |
| 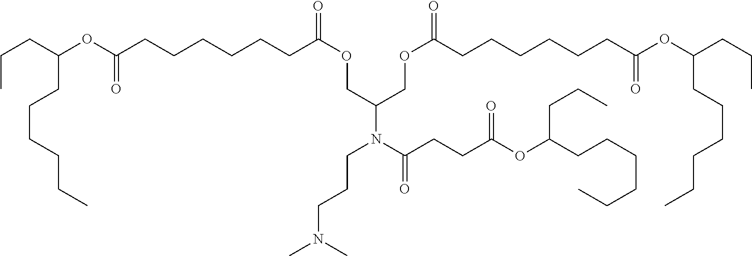 [SMILES: CCCCCCC(CCC)OC(=O)CCCCCCC(=O)OCC(COC(=O)CCCCCCC(=O)OC(CCC)CCCCCC)N(CCCN(C)C)C(=O)CCC(=O)OC(CCC)CCCCCC] | Lipid 17 |
| 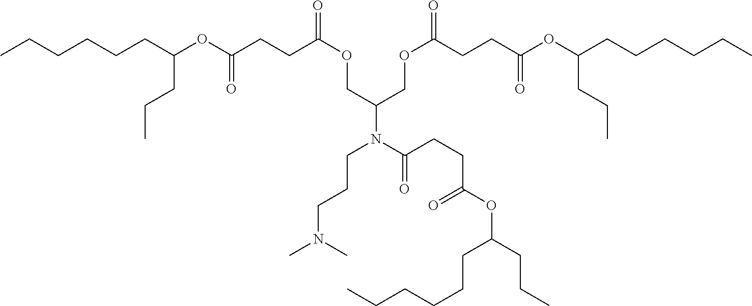 [SMILES: CCCCCCC(CCC)OC(=O)CCC(=O)OCC(COC(=O)CCC(=O)OC(CCC)CCCCCC)N(CCCN(C)C)C(=O)CCC(=O)OC(CCC)CCCCCC] | Lipid 17A |
| 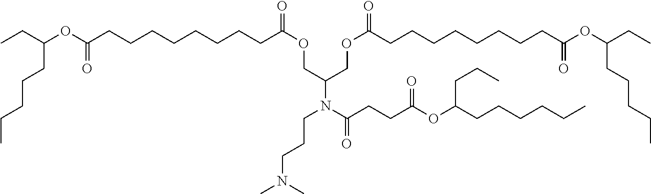 [SMILES: CCCCCCC(CCC)OC(=O)CCC(=O)N(CCCN(C)C)C(COC(=O)CCCCCCCCC(=O)OC(CC)CCCCC)COC(=O)CCCCCCCCC(=O)OC(CC)CCCCC] | Lipid 18 |
| 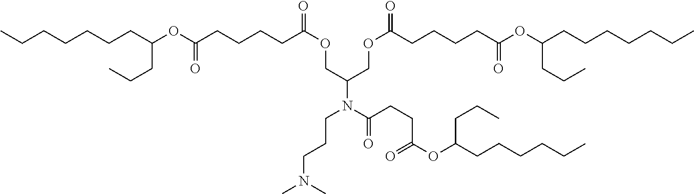 [SMILES: CCCCCCCC(CCC)OC(=O)CCCCC(=O)OCC(COC(=O)CCCCC(=O)OC(CCC)CCCCCCC)N(CCCN(C)C)C(=O)CCC(=O)OC(CCC)CCCCCC] | Lipid 18A |
| 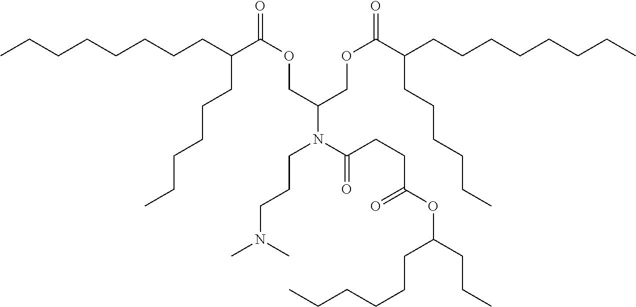 [SMILES: CCCCCCCCC(CCCCCC)C(=O)OCC(COC(=O)C(CCCCCC)CCCCCCCC)N(CCCN(C)C)C(=O)CCC(=O)OC(CCC)CCCCCC] | Lipid 19 |
| 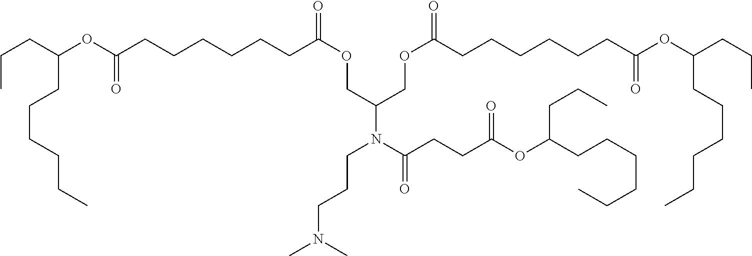 [SMILES: CCCCCCC(CCC)OC(=O)CCCCCCC(=O)OCC(COC(=O)CCCCCCC(=O)OC(CCC)CCCCCC)N(CCCN(C)C)C(=O)CCC(=O)OC(CCC)CCCCCC] | Lipid 19A |
| 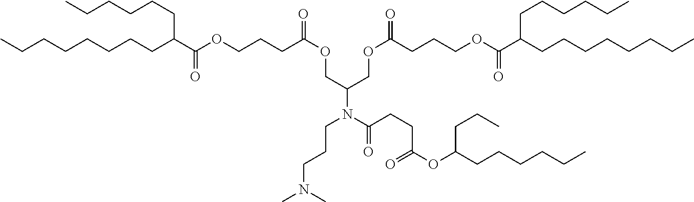 [SMILES: CCCCCCCCC(CCCCCC)C(=O)OCCCC(=O)OCC(COC(=O)CCCOC(=O)C(CCCCCC)CCCCCCCC)N(CCCN(C)C)C(=O)CCC(=O)OC(CCC)CCCCCC] | Lipid 20 |
| 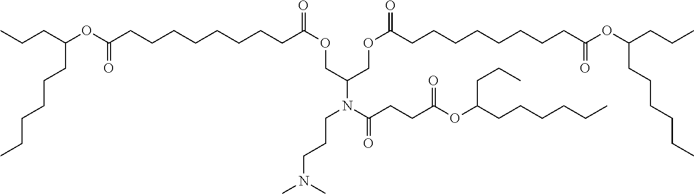 [SMILES: CCCCCCC(CCC)OC(=O)CCCCCCCCC(=O)OCC(COC(=O)CCCCCCCCC(=O)OC(CCC)CCCCCC)N(CCCN(C)C)C(=O)CCC(=O)OC(CCC)CCCCCC] | Lipid 20A |
| 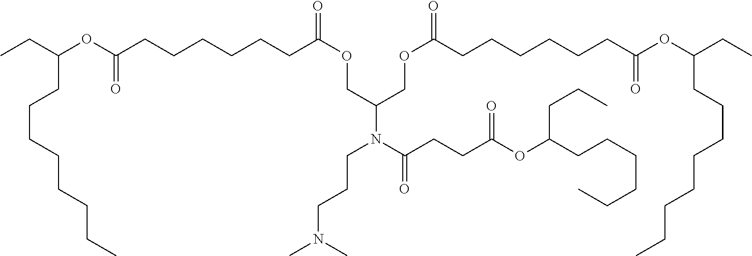 [SMILES: CCCCCCCCC(CC)OC(=O)CCCCCCC(=O)OCC(COC(=O)CCCCCCC(=O)OC(CC)CCCCCCCC)N(CCCN(C)C)C(=O)CCC(=O)OC(CCC)CCCCCC] | Lipid 21 |
| 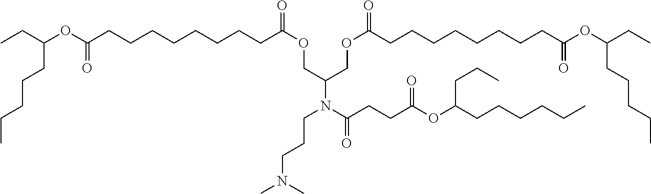 [SMILES: CCCCCCC(CCC)OC(=O)CCC(=O)N(CCCN(C)C)C(COC(=O)CCCCCCCCC(=O)OC(CC)CCCCC)COC(=O)CCCCCCCCC(=O)OC(CC)CCCCC] | Lipid 21A |
| 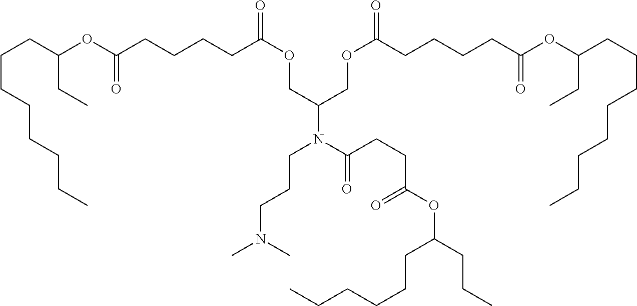 [SMILES: CCCCCCCCC(CC)OC(=O)CCCCC(=O)OCC(COC(=O)CCCCC(=O)OC(CC)CCCCCCCC)N(CCCN(C)C)C(=O)CCC(=O)OC(CCC)CCCCCC] | Lipid 22 |
| 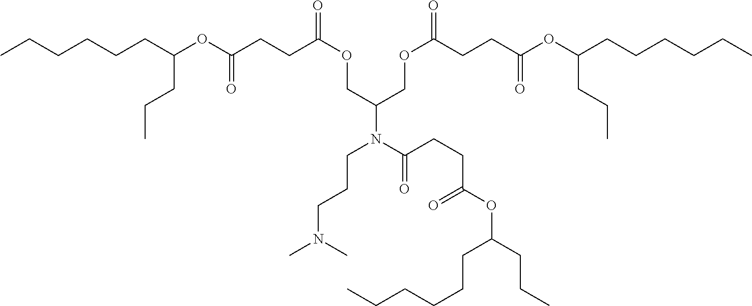 [SMILES: CCCCCCC(CCC)OC(=O)CCC(=O)OCC(COC(=O)CCC(=O)OC(CCC)CCCCCC)N(CCCN(C)C)C(=O)CCC(=O)OC(CCC)CCCCCC] | Lipid 23 |
| 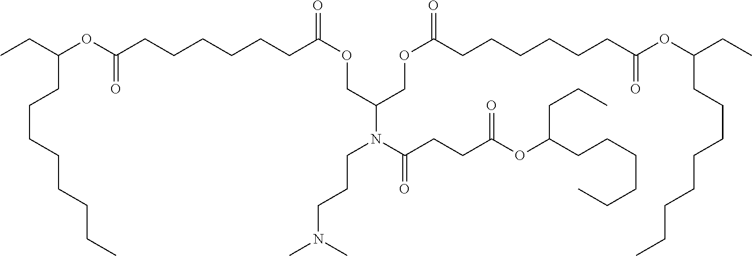 [SMILES: CCCCCCCCC(CC)OC(=O)CCCCCCC(=O)OCC(COC(=O)CCCCCCC(=O)OC(CC)CCCCCCCC)N(CCCN(C)C)C(=O)CCC(=O)OC(CCC)CCCCCC] | Lipid 23A |
| 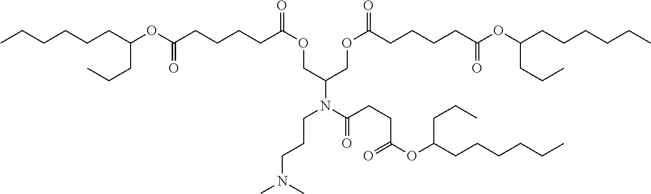 [SMILES: CCCCCCC(CCC)OC(=O)CCCCC(=O)OCC(COC(=O)CCCCC(=O)OC(CCC)CCCCCC)N(CCCN(C)C)C(=O)CCC(=O)OC(CCC)CCCCCC] | Lipid 24 |
| 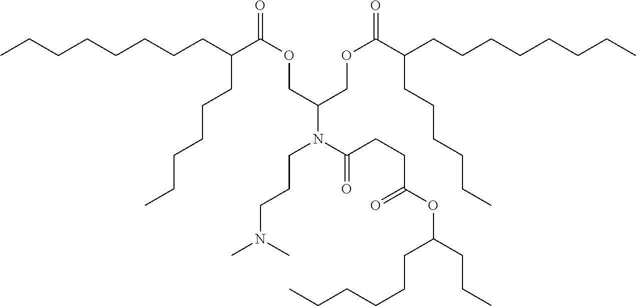 [SMILES: CCCCCCCCC(CCCCCC)C(=O)OCC(COC(=O)C(CCCCCC)CCCCCCCC)N(CCCN(C)C)C(=O)CCC(=O)OC(CCC)CCCCCC] | Lipid 24A |
| 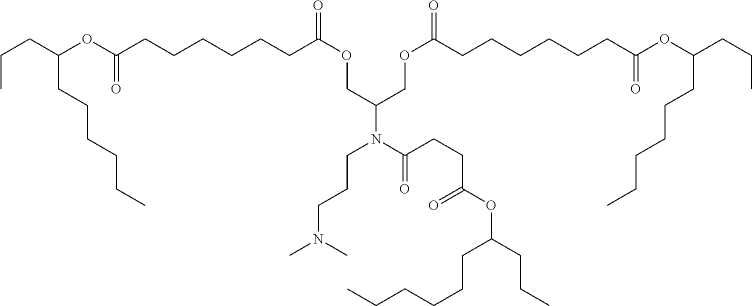 [SMILES: CCCCCCC(CCC)OC(=O)CCCCCCC(=O)OCC(COC(=O)CCCCCCC(=O)OC(CCC)CCCCCC)N(CCCN(C)C)C(=O)CCC(=O)OC(CCC)CCCCCC] | Lipid 25 |
| 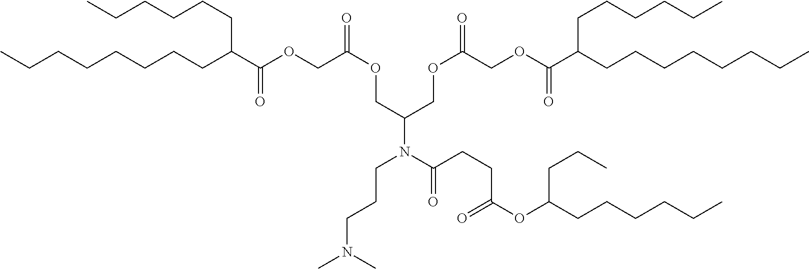 [SMILES: CCCCCCCCC(CCCCCC)C(=O)OCC(=O)OCC(COC(=O)COC(=O)C(CCCCCC)CCCCCCCC)N(CCCN(C)C)C(=O)CCC(=O)OC(CCC)CCCCCC] | Lipid 25A |
| 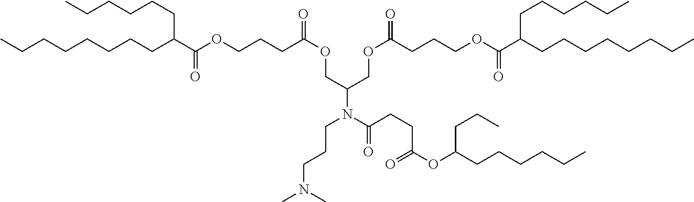 [SMILES: CCCCCCCCC(CCCCCC)C(=O)OCCCC(=O)OCC(COC(=O)CCCOC(=O)C(CCCCCC)CCCCCCCC)N(CCCN(C)C)C(=O)CCC(=O)OC(CCC)CCCCCC] | Lipid 26 |
| 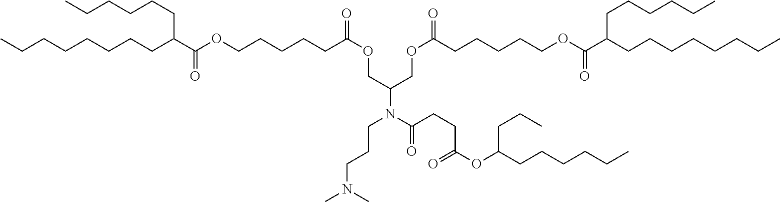 [SMILES: CCCCCCCCC(CCCCCC)C(=O)OCCCCCC(=O)OCC(COC(=O)CCCCCOC(=O)C(CCCCCC)CCCCCCCC)N(CCCN(C)C)C(=O)CCC(=O)OC(CCC)CCCCCC] | Lipid 27 |
| 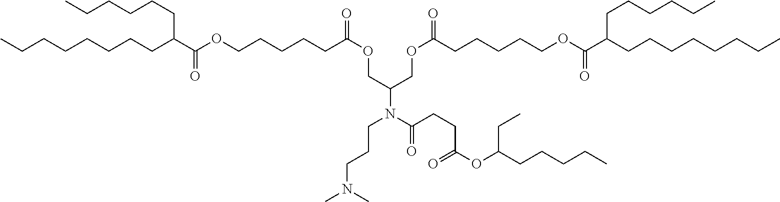 [SMILES: CCCCCCCCC(CCCCCC)C(=O)OCCCCCC(=O)OCC(COC(=O)CCCCCOC(=O)C(CCCCCC)CCCCCCCC)N(CCCN(C)C)C(=O)CCC(=O)OC(CC)CCCCC] | Lipid 28 |
| 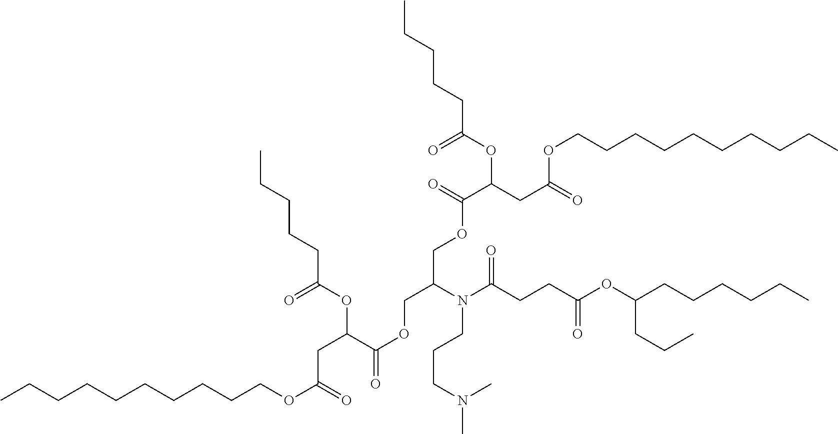 [SMILES: CCCCCCCCCCOC(=O)CC(OC(=O)CCCCC)C(=O)OCC(COC(=O)C(CC(=O)OCCCCCCCCCC)OC(=O)CCCCC)N(CCCN(C)C)C(=O)CCC(=O)OC(CCC)CCCCCC] | Lipid 29 |
| 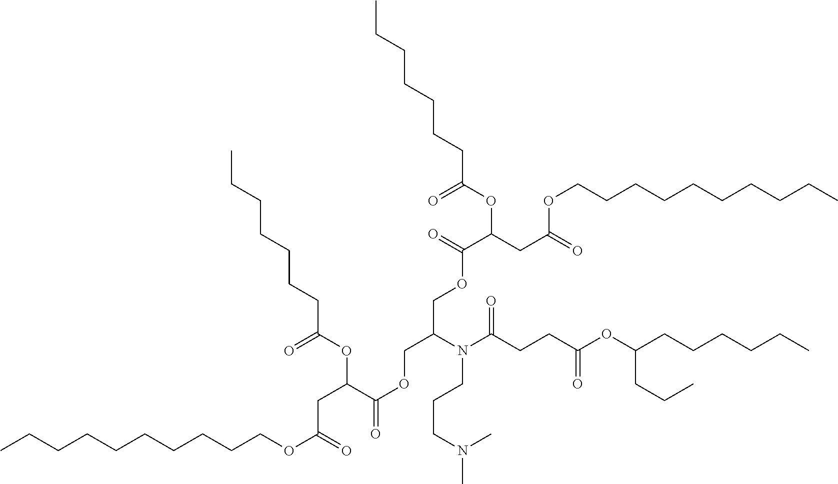 [SMILES: CCCCCCCCCCOC(=O)CC(OC(=O)CCCCCCC)C(=O)OCC(COC(=O)C(CC(=O)OCCCCCCCCCC)OC(=O)CCCCCCC)N(CCCN(C)C)C(=O)CCC(=O)OC(CCC)CCCCCC] | Lipid 30 |
| 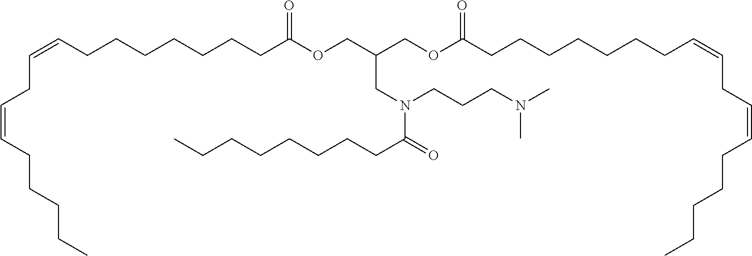 [SMILES: CCCCC/C=C\C/C=C\CCCCCCCC(=O)OCC(COC(=O)CCCCCCC/C=C\C/C=C\CCCCC)CN(CCCN(C)C)C(=O)CCCCCCCC] | Lipid 31 |
| 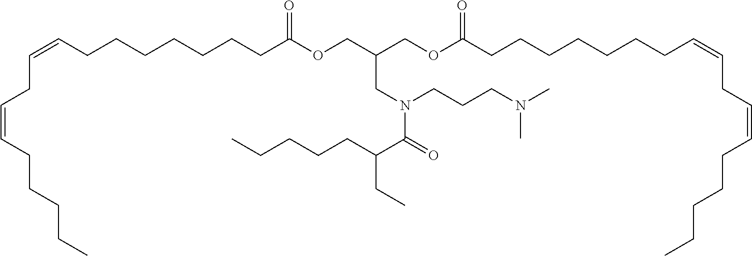 [SMILES: CCCCC/C=C\C/C=C\CCCCCCCC(=O)OCC(COC(=O)CCCCCCC/C=C\C/C=C\CCCCC)CN(CCCN(C)C)C(=O)C(CC)CCCCC] | Lipid 32 |
| 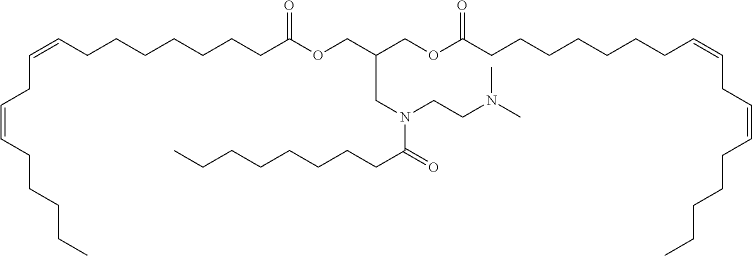 [SMILES: CCCCC/C=C\C/C=C\CCCCCCCC(=O)OCC(COC(=O)CCCCCCC/C=C\C/C=C\CCCCC)CN(CCN(C)C)C(=O)CCCCCCCC] | Lipid 33 |
| 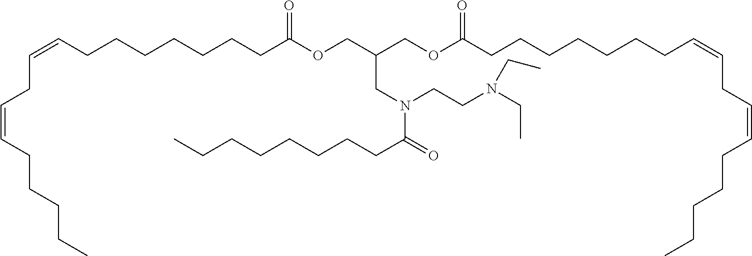 [SMILES: CCCCC/C=C\C/C=C\CCCCCCCC(=O)OCC(COC(=O)CCCCCCC/C=C\C/C=C\CCCCC)CN(CCN(CC)CC)C(=O)CCCCCCCC] | Lipid 34 |
| 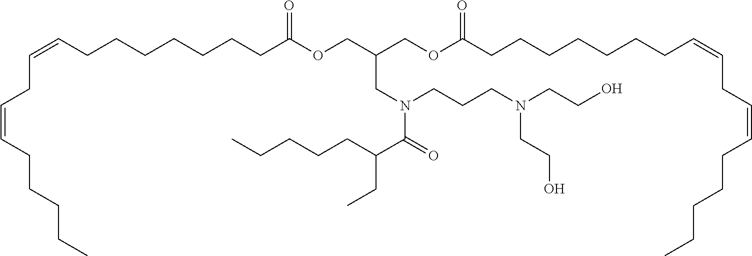 [SMILES: CCCCC/C=C\C/C=C\CCCCCCCC(=O)OCC(COC(=O)CCCCCCC/C=C\C/C=C\CCCCC)CN(CCCN(CCO)CCO)C(=O)C(CC)CCCCC] | Lipid 35 |
| 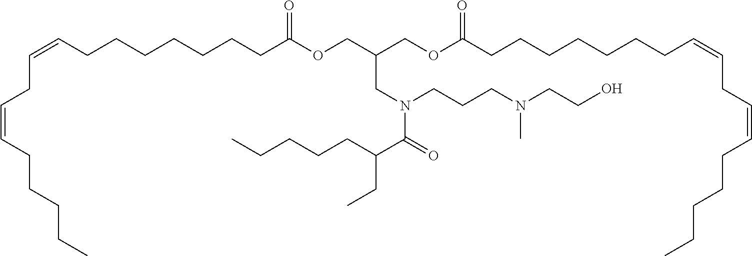 [SMILES: CCCCC/C=C\C/C=C\CCCCCCCC(=O)OCC(COC(=O)CCCCCCC/C=C\C/C=C\CCCCC)CN(CCCN(C)CCO)C(=O)C(CC)CCCCC] | Lipid 36 |
| 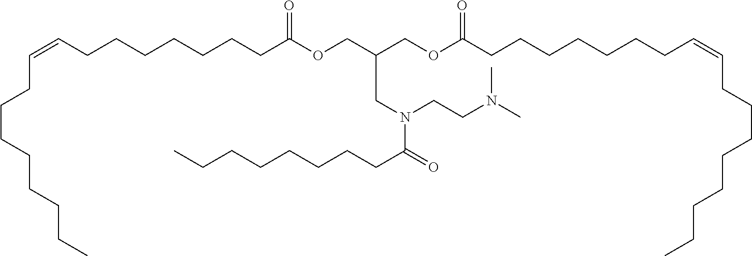 [SMILES: CCCCCCCC/C=C\CCCCCCCC(=O)OCC(COC(=O)CCCCCCC/C=C\CCCCCCCC)CN(CCN(C)C)C(=O)CCCCCCCC] | Lipid 37 |
| 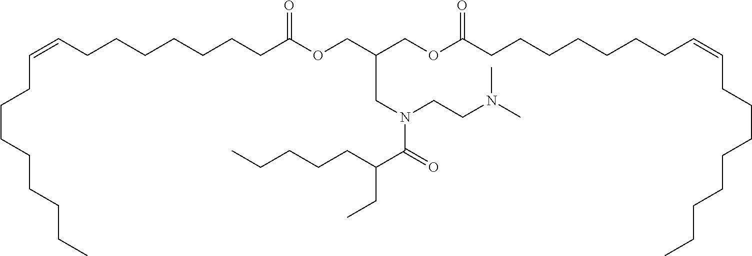 [SMILES: CCCCCCCC/C=C\CCCCCCCC(=O)OCC(COC(=O)CCCCCCC/C=C\CCCCCCCC)CN(CCN(C)C)C(=O)C(CC)CCCCC] | Lipid 38 |
| 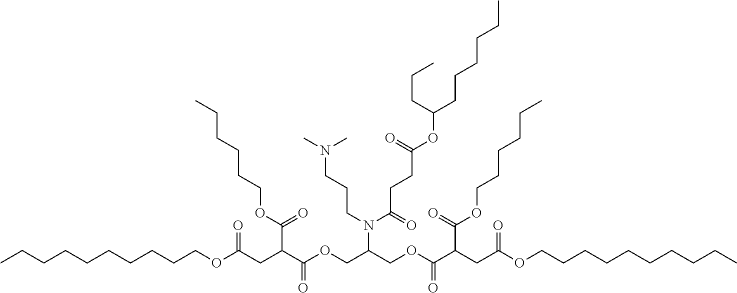 [SMILES: CCCCCCCCCCOC(=O)CC(C(=O)OCCCCCC)C(=O)OCC(COC(=O)C(CC(=O)OCCCCCCCCCC)C(=O)OCCCCCC)N(CCCN(C)C)C(=O)CCC(=O)OC(CCC)CCCCCC] | Lipid 37A |
| 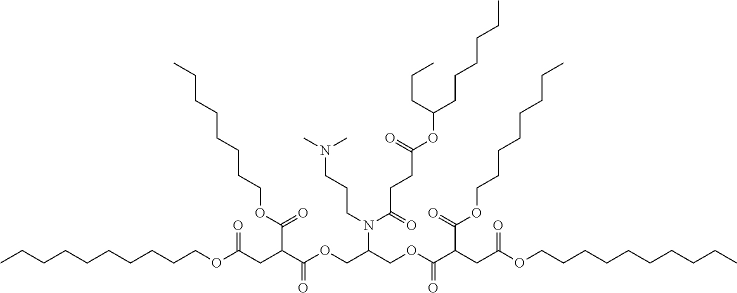 [SMILES: CCCCCCCCCCOC(=O)CC(C(=O)OCCCCCCCC)C(=O)OCC(COC(=O)C(CC(=O)OCCCCCCCCCC)C(=O)OCCCCCCCC)N(CCCN(C)C)C(=O)CCC(=O)OC(CCC)CCCCCC] | Lipid 38A |

Any variation or embodiment of R¹, R², R³, R^{1A}, R^{2A}, R^{3A}, R^{1A1}, R^{1A}, R^{1A3}, R^{2A1}, R^{2A2}, R^{2A3}, R^{3A1}, R^{3A2}, R^{3A3}, R^{a1}, R^{a2}, R^{3B}, R^{3B1}, R^{3B2}, R^{3B3}, R^{s1}, R^{s2}, R^{s3}, R^{s4}, R^{s5}, R^{s6}, R^{s7}, R^{s8}, R^{s9}, R^{s10}, R^{s11}, R^{s12}, R^{s13}, R^{s14}, or R^{s15} provided herein can be combined with every other variation or embodiment of R¹, R², R³, R^{1A}, R^{2A}, R^{3A}, R^{1A}, R^{1A2}, R^{1A3}, R^{2A}, R^{2A2}, R^{2A3}, R^{3A1}, R^{3A}, R^{3A3}, R^{a1}, R^{a2}, R^{3B}, R^{3B1}, R^{3B2}, R^{3B3}, R^{s1}, R^{s2}, R^{s3}, R^{s4}, RSs, R^{s6}, R^{s7}, R^{s8}, R^{s9}, R^{s10}, R^{s11}, R^{s12}, R^{s13}, R^{s14} or R^{s15}, as if each combination had been individually and specifically described.

The LNPs can be formulated using the methods and other components described below in the following sections.

### IV. Lipid Nanoparticle Compositions

The invention provides a lipid nanoparticle (LNP) composition comprising a lipid blend that contains an ionizable cationic lipid described herein and/or a lipid-immune cell targeting agent conjugate described herein. In certain embodiments, the lipid blend may comprise an ionizable, cationic lipid described herein and one or more of a sterol, a neutral phospholipid, a PEG-lipid, and a lipid-immune cell targeting group conjugate.

In certain embodiments, the ionizable, cationic lipid described herein may be present in the lipid blend in a range of 30-70 mole percent, 30-60 mole percent 30-50 mole percent, 40-70 mole percent, 40-60 mole percent, 40-50 mole percent, 50-70 mole percent, 50-60 mole percent, or of about 30 mole percent, about 35 mole percent, about 40 mole percent, about 45 mole percent, about 50 mole percent, about 55 mole percent, about 60 mole percent, about 65 mole percent, or about 70 mole percent.

### Sterol

In certain embodiments, the lipid blend of the lipid nanoparticle may comprise a sterol component, for example, one or more sterols selected from the group consisting of cholesterol, fecosterol, 0-sitosterol, ergosterol, campesterol, stigmasterol, stigmastanol, brassicasterol. In certain embodiments, the sterol is cholesterol.

The sterol (e.g., cholesterol) may be present in the lipid blend in a range of 20-70 mole percent, 20-60 mole percent, 20-50 mole percent, 30-70 mole percent, 30-60 mole percent, 30-50 mole percent, 40-70 mole percent, 40-60 mole percent, 40-50 mole percent, 50-70 mole percent, 50-60 mole percent, or about 20 mole percent, about 25 mole percent, about 30 mole percent, about 35 mole percent, about 40 mole percent, about 45 mole percent, about 50 mole percent, about 55 mole percent, about 60 mole percent or about 65 mole percent.

### Neutral Phospholipid

In certain embodiments, the lipid blend of the lipid nanoparticle may contain one or more neutral phospholipids. The neutral phospholipid can be selected from the group consisting of phosphatidylcholine, phosphatidylethanolamine, distearoyl-sn-glycero-3-phosphoethanolamine (DSPE), 1,2-distearoyl-sn-glycero-3-phosphocholine (DSPC), hydrogenated soy phosphatidylcholine (HSPC), 1,2-dioleoyl-sn-glycero-3-phosphoethanolamine (DOPE), 1,2-dioleoyl-sn-glycero-3-phosphocholine (DOPC), sphingomyelin (SM).

Other neutral phospholipids can be selected from the group consisting of distearoyl-phosphatidylethanolamine (DSPE), dimyrstoyl-phosphatidylethanolamine (DMPE), distearoyl-glycero-phosphocholine (DSPC), hydrogenated soy phosphatidylcholine (HSPC), dioleoyl-glycero-phosphoethanolamine (DOPE), dilinoleoyl-glycero-phosphocholine (DLPC), dimyristoyl-glycero-phosphocholine (DMPC), dioleoyl-glycero-phosphocholine (DOPC), dipalmitoyl-glycero-phosphocholine (DPPC), diundecanoyl-glycero-phosphocholine (DUPC), palmitoyl-oleoyl-glycero-phosphocholine (POPC), dioctadecenyl-glycero-phosphocholine, oleoyl-cholesterylhemisuccinoyl-glycero-phosphocholine, hexadecyl-glycero-phosphocholine, dilinolenoyl-glycero-phosphocholine, diarachidonoyl-glycero-3-phosphocholine, didocosahexaenoyl-glycero-phosphocholine, or sphingomyelin.

The neutral phospholipid may be present in the lipid blend in a range of 1-10 mole percent, 1-15 mole percent, 1-12 mole percent, 1-10 mole percent, 3-15 mole percent, 3-12 mole percent, 3-10 mole percent, 4-15 mole percent, 4-12 mole percent, 4-10 mole percent, 4-8 mole percent, 5-15 mole percent, 5-12 mole percent, 5-10 mole percent, 6-15 mole percent, 6-12 mole percent, 6-10 more percent, or about 1 mole percent, about 2 mole percent, about 3 mole percent, about 4 mole percent, about 5 mole percent, about 6 mole percent, about 7 mole percent, about 8 mole percent, about 9 mole percent, about 10 mole percent, about 11 mole percent, about 12 mole percent, about 13 mole percent, about 14 mole percent, or about 15 mole percent.

### PEG-Lipid

The lipid blend of the lipid nanoparticle may include one or more PEG or PEG-modified lipids. Such species may be alternately referred to as PEGylated lipids. A PEG lipid is a lipid modified with polyethylene glycol. As noted above, free PEG-lipids can be included in the lipid blend to reduce or eliminate non-specific binding via a targeting group when a lipid-immune cell targeting group is included in the lipid blend.

A PEG lipid may be selected from the non-limiting group consisting of PEG-modified phosphatidylethanolamines, PEG-modified phosphatidic acids, PEG-modified ceramides, PEG-modified dialkylamines, PEG-modified diacylglycerols, and PEG-modified dialkylglycerols. For example, a PEG lipid may be PEG-dioleoylgylcerol (PEG-DOG), PEG-dimyristoyl-glycerol (PEG-DMG), PEG-dipalmitoyl-glycerol (PEG-DPG), PEG-dilinoleoyl-glycero-phosphatidyl ethanolamine (PEG-DLPE), PEG-dimyrstoyl-phosphatidylethanolamine (PEG-DMPE), PEG-dipalmitoyl-phosphatidylethanolamine (PEG-DPPE), PEG-distearoylglycerol (PEG-DSG), PEG-diacylglycerol (PEG-DAG, e.g., PEG-DMG, PEG-DPG, and PEG-DSG), PEG-ceramide, PEG-distearoyl-glycero-phosphoglycerol (PEG-DSPG), PEG-dioleoyl-glycero-phosphoethanolamine (PEG-DOPE), 2-[(polyethylene glycol)-2000]-N,N-ditetradecylacetamide, or a PEG-distearoyl-phosphatidylethanolamine (PEG-DSPE) lipid.

In certain embodiments, the blend may contain a free PEG-lipid that can be selected from the group consisting of PEG-distearoylglycerol (PEG-DSG), PEG-diacylglycerol (PEG-DAG, e.g., PEG-DMG, PEG-DPG, and PEG-DSG), PEG-dimyristoyl-glycerol (PEG-DMG), PEG-distearoyl-phosphatidylethanolamine (PEG-DSPE) and PEG-dimyrstoyl-phosphatidylethanolamine (PEG-DMPE). In some embodiments, the free PEG-lipid comprises a diacylphosphatidylcholines comprising Dipalmitoyl (C16) chain or Distearoyl (C18) chain.

The PEG-lipid may be present in the lipid blend in a range of 1-10 mole percent, 1-8 mole percent, 1-7 mole percent, 1-6 mole percent, 1-5 mole percent, 1-4 mole percent, 1-3 mole percent, 2-8 mole percent, 2-7 mole percent, 2-6 mole percent, 2-5 mole percent, 2-4 mole percent, 2-3 mole percent, or about 1 mole percent, about 2 mole percent, about 3 mole percent, about 4 mole percent, or about 5 mole percent. In some embodiments, the PEG-lipid is a free PEG-lipid.

In some embodiments, the PEG-lipid may be present in the lipid blend in the range of 0.01-10 mole percent, 0.01-5 mole percent, 0.01-4 mole percent, 0.01-3 mole percent, 0.01-2 mole percent, 0.01-1 mole percent, 0.1-10 mole percent, 0.1-5 mole percent, 0.1-4 mole percent, 0.1-3 mole percent, 0.1-2 mole percent, 0.1-1 mole percent, 0.5-10 mole percent, 0.5-5 mole percent, 0.5-4 mole percent, 0.5-3 mole percent, 0.5-2 mole percent, 0.5-1 mole percent, 1-2 mole percent, 3-4 mole percent, 4-5 mole percent, 5-6 mole percent, or 1.25-1.75 mole percent. In some embodiments, the PET-lipid may be about 0.5 mole percent, about 1 mole percent, about 1.5 mole percent, about 2 mole percent, about 2.5 mole percent, about 3 mole percent, about 3.5 mole percent, about 4 mole percent, about 4.5 mole percent, about 5 mole percent, or about 5.5 mole percent of the lipid blend. In some embodiments, the PEG-lipid is a free PEG-lipid.

In some embodiments, the lipid anchor length of PEG-lipid is C14 (as in PEG-DMG). In some embodiments, the lipid anchor length of PEG-lipid is C16 (as in DPG). In some embodiments, the lipid anchor length of PEG-lipid is C18 (as in PEG-DSG). In some embodiments, the back bone or head group of PEG-lipid is diacyl glycerol or phosphoethanolamine. In some embodiments, the PEG-lipid is a free PEG-lipid.

A LNP of the present disclosure may comprise one or more free PEG-lipid that is not conjugated to an immune cell targeting group, and a PEG-lipid that is conjugated to immune cell targeting group. In some embodiments, the free PEG-lipid comprises the same or a different lipid as the lipid in the lipid-immune cell targeting group conjugate.

### Immune Cell Targeting Group Conjugate

In certain embodiments, the lipid blend can also include a lipid-immune cell targeting group conjugate.

The lipid-immune cell targeting group conjugate may be present in the lipid blend in a range of 0.001-0.5 mol percent, 0.001-0.1 mole percent, 0.01-0.5 mole percent, 0.05-0.5 mole percent, 0.1-0.5 mole percent, 0.1-0.3 mole percent, 0.1-0.2 mole percent, 0.2-0.3 mole percent, of about 0.01 mole percent, about 0.05 mole percent, about 0.1 mole percent, about 0.15 mole percent, about 0.2 mole percent, about 0.25 mole percent, about 0.3 mole percent, about 0.35 mole percent, about 0.4 mole percent, about 0.45 mole percent, or about 0.5 mole percent.

In addition to the lipids present in the lipid blend, the LNP compositions may further comprise a payload, for example, a payload described hereinbelow. In certain embodiments, the payload is a nucleic acid, for example, DNA or RNA, for example, an mRNA, transfer RNA (tRNA), a microRNA, or small interfering RNA (siRNA).

In certain embodiments, the number of the nucleotides in the nucleic acid is from about 400 to about 6000.

### Production of Lipid Nanoparticles

In some embodiments, the LNPs are produced by using either rapid mixing by an orbital vortexer or by microfluidic mixing. Orbital vortexer mixing is accomplished by rapid addition of lipids solution in ethanol to the aqueous solution of a nucleic acid of interest followed immediately by vortexing at 2,500 rpm. In some embodiments, the LNPs are produced using a microfluidic mixing step. In some embodiments, microfluidic mixing is achieved mixing the aqueous and organic streams at a controlled flow rates in a microfluidic channel using, e.g., a NanoAssemblr device and microfluidic chips featuring optimized mixing chamber geometry (Precision Nanosystems, Vancouver, BC). In some embodiments, the LNPs are produced using a microfluidic mixing step to rapidly mix the ethanolic lipid solution and aqueous nucleic acid solution, resulting in encapsulation of the nucleic acid in the solid lipid nanoparticles. The nanoparticle suspension is then buffer exchanged into an all aqueous buffer using membrane filtration device of choice for ethanol removal and nanoparticle maturation.

In certain embodiments, the resulting LNP compositions comprise a lipid blend containing, for example, from about 40 mole percent to about 60 mole percent of one or more ionizable cationic lipids described herein, from about 35 mole percent to about 50 mole percent of one or more sterols, from about 5 mole percent to about 15 mole percent of one or more neutral lipids, and from about 0.5 mole percent to about 5 mole percent of one or more PEG-lipids.

### Physical Properties of Lipid Nanoparticles

The characteristics of an LNP composition may depend on the components, their absolute or relative amounts, contained in a lipid nanoparticle (LNP) composition. Characteristics may also vary depending on the method and conditions of preparation of the LNP composition.

LNP compositions may be characterized by a variety of methods. For example, microscopy (e.g., transmission electron microscopy or scanning electron microscopy) may be used to examine the morphology and size distribution of an LNP composition. Dynamic light scattering or potentiometry (e.g., potentiometric titrations) may be used to measure zeta potentials. Dynamic light scattering may also be utilized to determine particle sizes. Instruments such as the Zetasizer Nano ZS (Malvern Instruments Ltd, Malvern, Worcestershire, UK) may also be used to measure multiple characteristics of an LNP composition, such as particle size, polydispersity index, and zeta potential. RNA encapsulated efficiency is determined by a combination of methods relying on RNA binding dyes (ribogreen, cybergreen to determine dye accessible RNA fraction) and LNP de-formulation followed by HPLC analysis for total RNA content.

In some embodiments, the LNP may have a mean diameter in the range of 1-250 nm, 1-200 nm, 1-150 nm, 1-100 nm, 50-250 nm, 50-200 nm, 50-150 nm, 50-100 nm, 75-250 nm, 75-200 nm, 75-150 nm, 75-100 nm, 100-250 nm, 100-200 nm, 100-150 nm. In certain embodiments, the LNP compositions may have a mean diameter of about 1 nm, about 10 nm, about 20 nm, about 30 nm, about 40 nm, about 50 nm, about 60 nm, about 70 nm, about 80 nm, about 90 nm, about 100 nm, about 110 nm, about 120 nm, about 130 nm, about 140 nm, about 150 nm, about 160 nm, about 170 nm, about 180 nm, about 190 nm, or about 200 nm. In some embodiments, the LNP has a mean diameter of about 100 nm.

In some embodiments, LNPs comprising an ionizable cationic lipid described herein, prepared and characterized using methods described herein, show average diameter change after a freeze-thaw of less than 1, 2, 3, 4, 5, 6, 7, 8, 9, 10, 15, 20, 25, 30, 35, or 40%. In some embodiments, LNPs comprising an ionizable cationic lipid described herein, prepared and characterized using methods described herein, show average diameter change after a freeze-thaw of less than 30%. In some embodiments, the freeze-thaw and diameter measurements are conducted with 10% sucrose in MES pH 6.5 buffer.

In some embodiments, LNPs comprising an ionizable cationic lipid described herein, prepared and characterized using methods described herein, show average diameter change upon targeting antibody insertion of less than 1, 2, 3, 4, 5, 6, 7, 8, 9, 10, 15, 20, 25, 30, 35, or 40%. In some embodiments, LNPs comprising an ionizable cationic lipid described herein, prepared and characterized using methods described herein, show average diameter change upon targeting antibody insertion of less than 15%. In some embodiments, the diameter change upon targeting antibody insertion is measured in pH 6.5 MES using a 37° C. incubation for 4 hours.

In some embodiments, LNPs comprising an ionizable cationic lipid described herein, prepared and characterized using methods described herein, have average LNP diameter of less than 50, 60, 70, 80, 90, 100, 110, 120, 130, 140, 150, 160, 170, 180, 190, or 200 nm. In some embodiments, LNPs comprising an ionizable cationic lipid described herein, prepared and characterized using methods described herein, have average LNP diameter of less than 100 nm.

Alternatively or in addition, the LNP compositions may have a polydispersity index in a range from 0.05-1, 0.05-0.75, 0.05-0.5, 0.05-0.4, 0.05-0.3, 0.05-0.2, 0.08-1, 0.08-0.75, 0.08-0.5, 0.08-0.4, 0.08-0.3, 0.08-0.2, 0.1-1, 0.1-0.75, 0.1-0.5, 0.1-0.4, 0.1-0.3, 0.1-0.2. In certain embodiments, the polydispersity index is in the range of 0.1-0.25, 0.1-0.2, 0.1-0.19, 0.1-0.18, 0.1-0.17, 0.1-0.16, or 0.1-0.15.

In some embodiments, the LNP compositions or LNPs comprising an ionizable cationic lipid described herein, prepared and characterized using methods described herein, have polydispersity of less than 0.4, 0.3, 0.25, 0.2, 0.15, 0.1, or 0.05. In some embodiments, LNPs comprising an ionizable cationic lipid described herein, prepared and characterized using methods described herein, have polydispersity of less than 0.25.

Alternatively or in addition, the LNP compositions may have a zeta potential of about −30 mV to about +30 mV. In certain embodiments, the LNP composition has a zeta potential of about −10 mV to about +20 mV. The zeta potential may vary as a function of pH. As a result, in certain embodiments, the LNP compositions may have a zeta potential of about 0 mV to about +30 mV or about +10 mV to +30 mV or about +20 mV to about +30 mV at pH 5.5 or pH 5, and/or a zeta potential of about −30 mV to about +5 mV or about −20 mV to about +15 mV at pH 7.4.

In some embodiments, the LNP compositions or LNPs comprising an ionizable cationic lipid described herein, prepared and characterized using methods described herein, have Zeta Potential at pH 7.4 greater than −10, −9, −8, −7, −6, −5.5, −5, −4.5, −4, −3.5, −3, −2.5, −2, −1.5, −1, or −0.5 mV. In some embodiments, the LNP compositions LNPs comprising an ionizable cationic lipid described herein, prepared and characterized using methods described herein, have Zeta Potential at pH 7.4 greater than −10 mV. In some embodiments, the LNP compositions LNPs comprising an ionizable cationic lipid described herein, prepared and characterized using methods described herein, have Zeta Potential at pH 7.4 greater than −1 mV. In some embodiments, the LNP compositions LNPs comprising an ionizable cationic lipid described herein, prepared and characterized using methods described herein, have Zeta Potential at pH 5.5 greater than −1, 0, 1, 2, 3, 4, 4.5, 5, 7.5, 10, 12.5, 15, 17.5, 20, 22.5, or 25 mV. In some embodiments, the LNP compositions LNPs comprising an ionizable cationic lipid described herein, prepared and characterized using methods described herein, have Zeta Potential at pH 5.5 greater than 5 mV. In some embodiments, the LNP compositions LNPs comprising an ionizable cationic lipid described herein, prepared and characterized using methods described herein, have Zeta Potential at pH 5.5 greater than 15 mV.

### V. Payloads

The LNP compositions may comprise an agent, for example, a nucleic acid molecule for delivery to a cell (e.g., an immune cell) or tissue, for example, a cell (e.g., an immune cell) or tissue in a subject.

The LNP compositions of the present invention may include a nucleic acid, for example, a DNA or RNA, such as an mRNA, tRNA, microRNA, siRNA, gRNA (guide RNA), circRNA (circular RNA), ribozymes, decoy RNA or dicer substrate siRNA. It is contemplated that nucleic acids can contain naturally occurring components, such as, naturally occurring bases, sugars or linkage groups (e.g., phosphodiester linkage groups) or may contain non-naturally occurring components or modifications, (e.g., thioester linkage groups). For example, the nucleic acid can be synthesized to contain base, sugar, linker modifications known to those skilled in the art. Furthermore, the nucleic acids can be linear or circular, or have any desired configuration. The LNP compositions can include multiple nucleic acid molecules, for example, multiple RNA molecules, which can be the same or different.

In certain embodiments, the payload is an mRNA. In certain embodiments, a particular LNP composition may contain a number of mRNA molecules that can be the same or different. In certain embodiments, one or more LNP compositions including one or more different mRNAs may be combined, and/or simultaneously contacted, with a cell. It is contemplated that an mRNA may include one or more of a stem loop, a chain terminating nucleoside, a polyA sequence, a polyadenylation signal, and/or a 5′ cap structure. The mRNA may encode a receptor, such as a chimeric antigen receptor (CAR), for use in for example, an immune disorder, inflammatory disorder or cancer. In addition, the mRNA may encode an antigen for use in a therapeutic or prophylactic vaccine, for example, for treating or preventing an infection by a pathogen, for example, a microbial or viral pathogen, or for reducing or ameliorating the side effects caused directly or indirectly by such an infection.

In certain embodiments, the LNP composition may include one or more other components including, but not limited to, one or more pharmaceutically acceptable excipients, small hydrophobic molecules, therapeutic agents, carbohydrates, polymers, permeability enhancing molecules, and surface altering agents.

In some embodiments, the wt/wt ratio of the lipid component to the payload (e.g., mRNA) in the resulting LNP composition is from about 1:1 to about 50:1. In certain embodiments, the wt/wt ratio of the lipid component to the payload (e.g., mRNA) in the resulting composition is from about 5:1 to about 50:1. In certain embodiments, the wt/wt ratio is from about 5:1 to about 40:1. In certain embodiments, the wt/wt ratio is from about 10:1 to about 40:1. In certain embodiments, the wt/wt ratio is from about 15:1 to about 25:1.

In certain embodiments, the encapsulation efficiency of the payload (e.g., mRNA) in the lipid nanoparticles is at least 50%. In certain embodiments, the encapsulation efficiency is at least 80%, at least 90% or, or greater than 90%.

In some embodiments, LNPs comprising an ionizable cationic lipid described herein, prepared and characterized using methods described herein, exhibit encapsulation efficiency of greater than 50, 55, 60, 65, 70, 75, 80, 82.5, 85, 87.5, 90, 92.5, 95, 97.5, or 99%. In some embodiments, LNPs comprising an ionizable cationic lipid described herein, prepared and characterized using methods described herein, exhibit encapsulation efficiency of greater than 87.5%. In some embodiments, LNPs comprising an ionizable cationic lipid described herein, prepared and characterized using methods described herein, exhibit dye accessible RNA of less than 50, 45, 40, 35, 30, 25, 20, 17.5, 15, 12.5, 10, 7.5, 5, 2.5, or 1%. In some embodiments, LNPs comprising an ionizable cationic lipid described herein, prepared and characterized using methods described herein, exhibit dye accessible RNA of less than 12.5%.

In some embodiments, LNPs comprising an ionizable cationic lipid described herein, prepared and characterized using methods described herein, exhibit total mRNA recovery of greater than 50, 55, 60, 65, 70, 75, 80, 85, 90, or 95%. In some embodiments, LNPs comprising an ionizable cationic lipid described herein, prepared and characterized using methods described herein, exhibit total mRNA recovery of greater than 80%.

### RNA Payload

In certain embodiments, the RNA payload is an mRNA, tRNA, microRNA, or siRNA payload.

In certain embodiments, the lipid nanoparticle compositions are optimized for the delivery of RNA, e.g., mRNA, to a target cell for translation within the cell. An mRNA may be a naturally or non-naturally occurring mRNA. An mRNA may include one or more modified nucleobases, nucleosides, or nucleotides.

The nucleobases may be selected from the non-limiting group consisting of adenine, guanine, uracil, cytosine, 7-methylguanine, 5-methylcytosine, 5-hydroxymethylcytosine, thymine, pseudouracil, dihydrouracil, N1-methylpseudouracil, hypoxanthine, and xanthine. In some embodiments, nucleobase is N1-methylpseudouracil.

A nucleoside of an mRNA is a compound including a sugar molecule (e.g., a 5-carbon or 6-carbon sugar, such as pentose, ribose, arabinose, xylose, glucose, galactose, or a deoxy derivative thereof) in combination with a nucleobase. A nucleoside may be a canonical nucleoside (e.g., adenosine, guanosine, cytidine, uridine, 5-methyluridine, deoxyadenosine, deoxyguanosine, deoxycytidine, deoxyuridine, and thymidine) or an analog thereof and may include one or more substitutions or modifications.

A nucleotide of an mRNA is a compound containing a nucleoside and a phosphate group or alternative group (e.g., boranophosphate, thiophosphate, selenophosphate, phosphonate, alkyl group, amidate, and glycerol). A nucleotide may be a canonical nucleotide (e.g., adenosine, guanosine, cytidine, uridine, 5-methyluridine, deoxyadenosine, deoxyguanosine, deoxycytidine, deoxyuridine, and thymidine monophosphates) or an analog thereof and may include one or more substitutions or modifications including but not limited to alkyl, aryl, halo, oxo, hydroxyl, alkyloxy, and/or thio substitutions; one or more fused or open rings; oxidation; and/or reduction of the nucleobase, sugar, and/or phosphate or alternative component. A nucleotide may include one or more phosphate or alternative groups. For example, a nucleotide may include a nucleoside and a triphosphate group. A “nucleoside triphosphate” (e.g., guanosine triphosphate, adenosine triphosphate, cytidine triphosphate, and uridine triphosphate) may refer to the canonical nucleoside triphosphate or an analog or derivative thereof and may include one or more substitutions or modifications as described herein.

An mRNA may include a 5′ untranslated region, a 3′ untranslated region, and/or a coding or translating sequence. An mRNA may include any number of base pairs, including tens, hundreds, or thousands of base pairs. Any number (e.g., all, some, or none) of nucleobases, nucleosides, or nucleotides may be an analog of a canonical species, substituted, modified, or otherwise non-naturally occurring. In certain embodiments, all of a particular nucleobase type may be modified. For example, all cytosine in an mRNA may be 5-methylcytosine. In certain embodiments, one or more or all uridine bases may be N1-methylpseudouridines.

In certain embodiments, an mRNA may include a 5′ cap structure, a chain terminating nucleotide, a stem loop, a polyA sequence, and/or a polyadenylation signal.

A cap structure or cap species is a compound including two nucleoside moieties joined by a linker and may be selected from a naturally occurring cap, a non-naturally occurring cap or a cap analog. A cap species may include one or more modified nucleosides and/or linker moieties. For example, a natural mRNA cap may include a guanine nucleotide and a guanine (G) nucleotide methylated at the 7 position joined by a triphosphate linkage at their 5′ positions, e.g., m7G(5′)ppp(5′)G, commonly written as m7GpppG. A cap species may also be an anti-reverse cap analog. A non-limiting list of possible cap species includes m7GpppG, m7Gpppm7G, m73′dGpppG, m7Gpppm7G, m73′dGpppG, and m27 02′GppppG.

Alternatively or in addition, an mRNA may include a chain terminating nucleoside. For example, a chain terminating nucleoside may include those nucleosides deoxygenated at the 2′ and/or 3′ positions of their sugar group. Such species may include 3′-deoxyadenosine (cordycepin), 3′-deoxyuridine, 3′-deoxycytosine, 3′-deoxyguanosine, 3′-deoxythymine, and 2′,3′-dideoxynucleosides, such as 2′,3′-dideoxyadenosine, 2′,3′-dideoxyuridine, 2′,3′-dideoxycytosine, 2′,3′-dideoxyguanosine, and 2′,3′-dideoxythymine.

Alternatively or in addition, an mRNA may include a stem loop, such as a histone stem loop. A stem loop may include 1, 2, 3, 4, 5, 6, 7, 8, or more nucleotide base pairs. For example, a stem loop may include 4, 5, 6, 7, or 8 nucleotide base pairs. A stem loop may be located in any region of an mRNA. For example, a stem loop may be located in, before, or after an untranslated region (a 5′ untranslated region or a 3′ untranslated region), a coding region, or a polyA sequence or tail.

Alternatively or in addition, an mRNA may include a polyA sequence and/or polyadenylation signal. A polyA sequence may be comprised entirely or mostly of adenine nucleotides or analogs or derivatives thereof. A polyA sequence may be a tail located adjacent to a 3′ untranslated region of an mRNA.

An mRNA may encode any polypeptide of interest, including any naturally or non-naturally occurring or otherwise modified polypeptide. A polypeptide encoded by an mRNA may be of any size and may have any secondary structure or activity. In some embodiments, a polypeptide encoded by an mRNA may have a therapeutic effect when expressed in a cell. In some embodiments, the mRNA may encode an antibody, enzyme, growth factor, hormone, cytokine, viral protein (e.g., a viral capsid protein), antigen, vaccine, or receptor. In some embodiments, the mRNA may encode an engineered receptor such as a CAR or an antigen for use in a therapeutic vaccine (e.g., a cancer vaccine) or a prophylactic vaccine (e.g., a vaccine for minimizing the risk or severity of an infection by a microbial or viral pathogen). In some embodiments, the mRNA encodes a polypeptide capable of regulating immune response in the immune cell. In some embodiments, the mRNA encodes a polypeptide capable of reprogramming the immune cell. In some embodiments, the mRNA encodes a synthetic T cell receptor (synTCR) or a Chimeric Antigen Receptor (CAR).

A lipid composition may be designed for one or more specific applications or targets. For example, an LNP composition may be designed to deliver mRNA to a particular cell, tissue, organ, or system or group thereof in a mammal's body, such as the renal system. Physiochemical properties of LNP compositions may be altered in order to increase selectivity for particular target site within a subject. For instance, particle sizes may be adjusted based on the fenestration sizes of different organs. The mRNA included in an LNP composition may also depend on the desired delivery target or targets. For example, an mRNA may be selected for a particular indication, condition, disease, or disorder and/or for delivery to a particular cell, tissue, organ, or system or group thereof (e.g., localized or specific delivery).

The amount of mRNA in a lipid composition may depend on the size, sequence, and other characteristics of the mRNA. The amount of mRNA in an LNP may also depend on the size, composition, desired target, and other characteristics of the LNP composition. The relative amounts of mRNA and other elements (e.g., lipids) may also vary. The amount of mRNA in an LNP composition may, for example, be measured using absorption spectroscopy (e.g., ultraviolet-visible spectroscopy).

In some embodiments, the one or more mRNAs, lipids, and polymers and amounts thereof may be selected to provide a specific N:P ratio (the ratio of positively-chargeable lipid or polymer amine (N=nitrogen) groups to negatively-charged nucleic acid phosphate (P) groups). The N:P ratio of the composition refers to the molar ratio of nitrogen atoms in one or more lipids to the number of phosphate groups in an mRNA. In general, a lower N:P ratio is preferred. A N:P ratio may be dependent on a specific lipid and its pKa. In certain embodiments, the mRNA and LNP composition, and/or their relative amounts may be selected to provide an N:P ratio from about 1:1 to about 30:1, or from about 1:1 to about 20:1. In certain embodiments, the N:P ratio can be, for example, 1:1, 2:1, 3:1, 4:1, 5:1, 6:1, 7:1, or 8:1. In certain embodiments, the N:P ratio may be from about 2:1 to about 5:1. In certain embodiments, the N:P ratio may be about 4:1. In other embodiments, the N:P ratio is from about 4:1 to about 8:1. For example, the N:P ratio may be about 4:1, about 4.5:1, about 4.6:1, about 4.7:1, about 4.8:1, about 4.9:1, about 5.0:1, about 5.1:1, about 5.2:1, about 5.3:1, about 5.4:1, about 5.5:1, about 5.6:1, about 5.7:1, about 6.0:1, about 6.5:1, or about 7.0:1.

The amount of mRNA in a nanoparticle composition may depend on the size, sequence, and other characteristics of the mRNA. The amount of mRNA in a nanoparticle composition may also depend on the size, composition, desired target, and other characteristics of the nanoparticle composition. The relative amounts of mRNA and other elements (e.g., lipids) may also vary. In some embodiments, the wt/wt ratio of the lipid component to an mRNA in a nanoparticle composition may be from about 5:1 to about 50:1, such as 5:1, 6:1, 7:1, 8:1, 9:1, 10:1, 11:1, 12:1, 13:1, 14:1, 15:1, 16:1, 17:1, 18:1, 19:1, 20:1, 25:1, 30:1, 35:1, 40:1, 45:1, and 50:1. For example, the wt/wt ratio of the lipid component to an mRNA may be from about 10:1 to about 40:1. The amount of mRNA in a nanoparticle composition may, for example, be measured using absorption spectroscopy (e.g., ultraviolet-visible spectroscopy).

The efficiency of encapsulation of an mRNA describes the amount of mRNA that is encapsulated or otherwise associated with a lipid composition after preparation, relative to the initial amount provided. The encapsulation efficiency is desirably high (e.g., close to 100%). The encapsulation efficiency may be measured, for example, by comparing the amount of mRNA in a solution containing the lipid composition before and after breaking up the LNP composition with one or more organic solvents or detergents. Fluorescence may be used to measure the amount of free mRNA in a solution. For the LNP compositions of the invention, the encapsulation efficiency of an mRNA may be at least 50%, for example 50%, 55%, 60%, 65%, 70%, 75%, 80%, 85%, 90%, 91%, 92%, 93%, 94%, 95%, 96%, 97%, 98%, 99%, or 100%. In certain embodiments, the encapsulation efficiency may be at least 80%.

### VI. Formulation and Mode of Delivery

LNP compositions of the invention may be formulated in whole or in part as a pharmaceutical composition. The pharmaceutical compositions may further include one or more pharmaceutically acceptable excipients or accessory ingredients such as those described herein. General guidelines for the formulation and manufacture of pharmaceutical compositions and agents are available, for example, in Remington's (2006) supra. Conventional excipients and accessory ingredients may be used in any pharmaceutical composition of the invention, except insofar as any conventional excipient or accessory ingredient may be incompatible with one or more components of an LNP composition of the invention. An excipient or accessory ingredient may be incompatible with a component of an LNP composition if its combination with the component may result in any undesirable biological effect or otherwise deleterious effect.

In some embodiments, one or more excipients or accessory ingredients may make up greater than 50% of the total mass or volume of a pharmaceutical composition including an LNP composition of the invention. For example, the one or more excipients or accessory ingredients may make up 30%, 40%, 50%, 60%, 70%, 80%, 90%, or more of a pharmaceutical composition. In certain embodiments, the excipient is approved for use in humans and for veterinary use, for example, by United States Food and Drug Administration. In certain embodiments, the excipient is pharmaceutical grade. In certain embodiments, an excipient meets the standards of the United States Pharmacopoeia (USP), the European Pharmacopoeia (EP), the British Pharmacopoeia, and/or the International Pharmacopoeia.

Relative amounts of the one or more lipids or LNPs, one or more pharmaceutically acceptable excipients, and/or any additional ingredients in a pharmaceutical composition will vary, depending upon the identity, size, and/or condition of the subject treated and further depending upon the route by which the composition is to be administered.

Lipid compositions and/or pharmaceutical compositions including one or more LNP compositions may be administered to any subject, including a human patient that may benefit from a therapeutic effect provided by the delivery of a nucleic acid, e.g., an RNA (e.g., mRNA, tRNA or siRNA) to one or more particular cells, tissues, organs, or systems or groups thereof, such as the renal system. Although the descriptions provided herein of LNP compositions and pharmaceutical compositions including LNP compositions are principally directed to compositions which are suitable for administration to humans, it will be understood by the skilled artisan that such compositions are generally suitable for administration to any other mammal. Modification of compositions suitable for administration to humans in order to render the compositions suitable for administration to various animals is understood.

A pharmaceutical composition in accordance with the present disclosure may be prepared, packaged, and/or sold in bulk, as a single unit dose, and/or as a plurality of single unit doses. As used herein, a “unit dose” is discrete amount of the pharmaceutical composition comprising a predetermined amount of the active ingredient (e.g., the payload).

Pharmaceutical compositions of the invention may be prepared in a variety of forms suitable for a variety of routes and methods of administration. For example, pharmaceutical compositions of the invention may be prepared in liquid dosage forms (e.g., emulsions, microemulsions, nanoemulsions, solutions, suspensions, syrups, and elixirs), injectable forms, solid dosage forms (e.g., capsules, tablets, pills, powders, and granules), dosage forms for topical and/or transdermal administration (e.g., ointments, pastes, creams, lotions, gels, powders, solutions, sprays, inhalants, and patches), suspensions, powders, and other forms.

Liquid dosage forms for oral and parenteral administration include, but are not limited to, pharmaceutically acceptable emulsions, microemulsions, nanoemulsions, solutions, suspensions, syrups, and/or elixirs. In addition to active ingredients, liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, oral compositions can include adjuvants such as wetting agents, emulsifying and suspending agents, sweetening, flavoring, and/or perfuming agents.

Injectable preparations, for example, sterile injectable aqueous or oleaginous suspensions may be formulated according to the known art using suitable dispersing agents, wetting agents, and/or suspending agents. Sterile injectable preparations may be sterile injectable solutions, suspensions, and/or emulsions in nontoxic parenterally acceptable diluents and/or solvents, for example, as a solution in 1,3-butanediol. Among the acceptable vehicles and solvents that may be employed are water, Ringer's solution, U.S.P., and isotonic sodium chloride solution. Sterile, fixed oils are conventionally employed as a solvent or suspending medium. For this purpose any bland fixed oil can be employed including synthetic mono- or diglycerides. Fatty acids such as oleic acid can be used in the preparation of injectables.

Injectable formulations can be sterilized, for example, by filtration through a bacterial-retaining filter, and/or by incorporating sterilizing agents in the form of sterile solid compositions which can be dissolved or dispersed in sterile water or other sterile injectable medium prior to use.

### Other Components

In addition, it is contemplated that the pharmaceutical compositions may include one or more components in addition to those described hereinabove.

The pharmaceutical compositions may also include one or more permeability enhancer molecules, carbohydrates, polymers, therapeutic agents, surface altering agents, or other components. A permeability enhancer molecule may be a molecule described, for example, in U.S. patent application publication No. 2005/0222064. Carbohydrates may include simple sugars (e.g., glucose) and polysaccharides (e.g., glycogen and derivatives and analogs thereof).

The pharmaceutical compositions may also contain a surface altering agent, including for example, anionic proteins (e.g., bovine serum albumin), surfactants (e.g., cationic surfactants such as dimethyldioctadecyl-ammonium bromide), sugars or sugar derivatives (e.g., cyclodextrin), polymers (e.g., heparin, polyethylene glycol, and poloxamer), mucolytic agents (e.g., acetylcysteine, mugwort, bromelain, papain, clerodendrum, bromhexine, carbocisteine, eprazinone, mesna, ambroxol, sobrerol, domiodol, letosteine, stepronin, tiopronin, gelsolin, thymosin β4, dornase alfa, neltenexine, and erdosteine), and DNases (e.g., rhDNase). A surface altering agent may be disposed within and/or upon the surface of a composition described herein.

In addition to these components, a pharmaceutical composition containing an LNP composition of the invention may include any substance useful in pharmaceutical compositions. For example, the pharmaceutical composition may include one or more pharmaceutically acceptable excipients or accessory ingredients such as, but not limited to, one or more solvents, dispersion media, diluents, dispersion aids, suspension aids, granulating aids, disintegrants, fillers, glidants, liquid vehicles, binders, surface active agents, isotonic agents, thickening or emulsifying agents, buffering agents, lubricating agents, oils, preservatives, and other species. Excipients such as waxes, butters, coloring agents, coating agents, flavorings, and perfuming agents may also be included. Pharmaceutically acceptable excipients are well known in the art (see, e.g., Remington's (2006) supra).

Dispersing agents may be selected from the non-limiting list consisting of potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (VEEGUM®), sodium lauryl sulfate, quaternary ammonium compounds, and/or combinations thereof.

Surface active agents and/or emulsifiers may include, but are not limited to, natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite [aluminum silicate] and VEEGUM® [magnesium aluminum silicate]), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate [TWEEN®20], polyoxyethylene sorbitan [TWEEN® 60], polyoxyethylene sorbitan monooleate [TWEEN®80], sorbitan monopalmitate [SPAN®40], sorbitan monostearate [SPAN®60], sorbitan tristearate [SPAN®65], glyceryl monooleate, sorbitan monooleate [SPAN®80]), polyoxyethylene esters (e.g., polyoxyethylene monostearate [MYRJ® 45], polyoxyethylene hydrogenated castor oil, polyethoxylated castor oil, polyoxymethylene stearate, and SOLUTOL®), sucrose fatty acid esters, polyethylene glycol fatty acid esters (e.g., CREMOPHOR®), polyoxyethylene ethers, (e.g., polyoxyethylene lauryl ether [BRIJ® 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, PLURONIC®F 68, POLOXAMER® 188, cetrimonium bromide, cetylpyridinium chloride, benzalkonium chloride, docusate sodium, and/or combinations thereof.

Examples of preservatives may include, but are not limited to, antioxidants, chelating agents, antimicrobial preservatives, antifungal preservatives, alcohol preservatives, acidic preservatives, and/or other preservatives. Examples of antioxidants include, but are not limited to, alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and/or sodium sulfite. Examples of chelating agents include ethylenediaminetetraacetic acid (EDTA), citric acid monohydrate, disodium edetate, dipotassium edetate, edetic acid, fumaric acid, malic acid, phosphoric acid, sodium edetate, tartaric acid, and/or trisodium edetate. Examples of antimicrobial preservatives include, but are not limited to, benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and/or thimerosal. Examples of antifungal preservatives include, but are not limited to, butyl paraben, methyl paraben, ethyl paraben, propyl paraben, benzoic acid, hydroxybenzoic acid, potassium benzoate, potassium sorbate, sodium benzoate, sodium propionate, and/or sorbic acid. Examples of alcohol preservatives include, but are not limited to, ethanol, polyethylene glycol, benzyl alcohol, phenol, phenolic compounds, bisphenol, chlorobutanol, hydroxybenzoate, and/or phenylethyl alcohol. Examples of acidic preservatives include, but are not limited to, vitamin A, vitamin C, vitamin E, beta-carotene, citric acid, acetic acid, dehydroascorbic acid, ascorbic acid, sorbic acid, and/or phytic acid. Other preservatives include, but are not limited to, tocopherol, tocopherol acetate, deteroxime mesylate, cetrimide, butylated hydroxyanisole (BHA), butylated hydroxytoluene (BHT), ethylenediamine, sodium lauryl sulfate (SLS), sodium lauryl ether sulfate (SLES), sodium bisulfite, sodium metabisulfite, potassium sulfite, potassium metabisulfite.

Examples of buffering agents include, but are not limited to, citrate buffer solutions, acetate buffer solutions, phosphate buffer solutions, ammonium chloride, calcium carbonate, calcium chloride, calcium citrate, calcium glubionate, calcium gluceptate, calcium gluconate, d-gluconic acid, calcium glycerophosphate, calcium lactate, calcium lactobionate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, amino-sulfonate buffers (e.g., HEPES), magnesium hydroxide, aluminum hydroxide, alginic acid, pyrogen-free water, isotonic saline, Ringer's solution, ethyl alcohol, and/or combinations thereof.

In certain embodiments, the lipid nanoparticle compositions and formulations thereof are adapted for administration intravenously, intramuscularly, intradermally, subcutaneously, intra-arterially, intra-tumor, or by inhalation. In certain embodiments, a dose of about 0.001 mg/kg to about 10 mg/kg is administered to a subject. Compositions in accordance with the present disclosure may be formulated in dosage unit form for ease of administration and uniformity of dosage. It will be understood, however, that the total daily usage of a composition of the present disclosure will be decided by an attending physician within the scope of sound medical judgment.

The specific therapeutically effective, prophylactically effective, or otherwise appropriate dose level (e.g., for imaging) for any particular patient will depend upon a variety of factors including the severity and identify of a disorder being treated, if any; the one or more mRNAs employed; the specific composition employed; the age, body weight, general health, sex, and diet of the patient; the time of administration, route of administration, and rate of excretion of the specific pharmaceutical composition employed; the duration of the treatment; drugs used in combination or coincidental with the specific pharmaceutical composition employed; and like factors well known in the medical arts.

### VII. Methods

The present disclosure provides methods of delivering a payload to a target cell or tissue, for example, a target cell or tissue in a subject, and LNPs or pharmaceutical compositions containing the LNPs for use in such methods. Any disclosure herein of a method of, e.g., treating a disease or disorder or, e.g., delivering a nucleic acid to a cell or, e.g., producing a polypeptide of interest in a cell should be interpreted also as a disclosure of an LNP or pharmaceutical composition comprising said LNP for use in such methods.

In certain embodiments, the invention provides a method of producing a polypeptide of interest (e.g., a protein of interest) in a mammalian cell, and LNPs or pharmaceutical compositions containing the LNPs for use in such methods. Methods of producing polypeptides in such a cell involve contacting a cell with an LNP composition comprising an RNA of interest (e.g., an mRNA encoding the polypeptide of interest (e.g., a protein of interest). Upon contacting the cell with the LNP composition, the mRNA may be taken up and translated in the cell to produce the polypeptide of interest.

In general, the step of contacting a mammalian cell with an LNP composition including an mRNA encoding a polypeptide of interest may be performed in vivo, ex vivo, or in vitro. The amount of an LNP composition contacted with a cell, and/or the amount of mRNA therein, may depend on the type of cell or tissue being contacted, the means of administration, the physiochemical characteristics of the LNP composition and the mRNA (e.g., size, charge, and chemical composition) therein, and other factors. In general, an effective amount of the LNP composition will allow for efficient polypeptide production in the cell. Metrics for efficiency may include polypeptide translation (indicated by polypeptide expression), level of mRNA degradation, and immune response indicators.

The step of contacting an LNP composition including an mRNA with a cell may involve or cause transfection where the LNP composition may fuse with the membrane of cell to permit the delivery of the mRNA into the cell. Upon introduction into the cytoplasm of the cell, the mRNA is then translated into a protein or peptide via the protein synthesis machinery within the cytoplasm of the cell.

In certain embodiments, the LNP compositions described herein may be used to deliver therapeutic or prophylactic agents to a subject. For example, an mRNA included in an LNP composition may encode a polypeptide and produce the therapeutic or prophylactic polypeptide upon contacting and/or entry (e.g., transfection) into a cell. In certain embodiments, an mRNA included in an LNP composition of the invention may encode a polypeptide that may improve or increase the immunity of a subject.

In certain embodiments, contacting a cell with an LNP composition including an mRNA may reduce the innate immune response of a cell to an exogenous nucleic acid. A cell may be contacted with a first LNP composition including a first amount of a first exogenous mRNA including a translatable region and the level of the innate immune response of the cell to the first exogenous mRNA may be determined. Subsequently, the cell may be contacted with a second composition including a second amount of the first exogenous mRNA, the second amount being a lesser amount of the first exogenous mRNA compared to the first amount. Alternatively, the second composition may include a first amount of a second exogenous mRNA that is different from the first exogenous mRNA. The steps of contacting the cell with the first and second compositions may be repeated one or more times.

Additionally, efficiency of polypeptide production in the cell may be optionally determined, and the cell may be re-contacted with the first and/or second composition repeatedly until a target protein production efficiency is achieved.

The present disclosure provides methods of delivering a nucleic acid (e.g., an mRNA) to a mammalian cell or tissue, for example, a mammalian cell or tissue in a subject. Delivery of an mRNA to such a cell or tissue involves administering an LNP composition including the mRNA to a subject, for example, by injection, e.g., via intramuscular injection or intravascular delivery into the subject. After administration, the LNP can target and/or contact a cell, for example, an immune cell, such as a T-cell. Upon contacting the cell with the LNP composition, a translatable mRNA may be translated in the cell to produce a polypeptide of interest.

In certain embodiments, an LNP composition of the invention may target a particular type or class of cells. This targeting may be facilitated using the lipids described herein to form LNPs, which may also include a targeting group for targeting cells of interest. In certain, embodiments, specific delivery may result in a greater than 2 fold, 5 fold, 10 fold, 15 fold, or 20 fold increase in the amount of mRNA to the targeted destination (e.g., cells that express or express at high levels the receptor of interest which binds to the immune cell targeting group of the LNPs) as compared to another destinations (e.g., cells that either do not express or only express at low levels the receptor of interest).

LNP compositions of the invention may be useful for treating a disease, disorder, or condition characterized by missing or aberrant protein or polypeptide activity. Upon delivery of an mRNA encoding the missing or aberrant polypeptide to a cell, translation of the mRNA may produce the polypeptide, thereby reducing or eliminating an issue caused by the absence of or aberrant activity caused by the polypeptide. Because translation may occur rapidly, the methods and compositions of the invention may be useful in the treatment of acute diseases, disorders, or conditions such as sepsis, stroke, and myocardial infarction. An mRNA included in an LNP composition of the invention may also be capable of altering the rate of transcription of a given species, thereby affecting gene expression.

Diseases, disorders, and/or conditions characterized by dysfunctional or aberrant protein or polypeptide activity for which a composition of the invention may be administered include, but are not limited to, cancer and proliferative diseases, genetic diseases (e.g., cystic fibrosis), autoimmune diseases, diabetes, neurodegenerative diseases, cardio- and reno-vascular diseases, and metabolic diseases. Multiple diseases, disorders, and/or conditions may be characterized by missing (or substantially diminished such that proper protein function does not occur) protein activity. Such proteins may not be present, or they may be essentially non-functional. A specific example of a dysfunctional protein is the missense mutation variants of the cystic fibrosis transmembrane conductance regulator (CFTR) gene, which produce a dysfunctional protein variant of CFTR protein, which causes cystic fibrosis. The present disclosure provides a method for treating such diseases, disorders, and/or conditions in a subject by administering an LNP composition including an mRNA and a lipid component including KL10, a phospholipid (optionally unsaturated), a PEG lipid, and a structural lipid, wherein the m RNA encodes a polypeptide that antagonizes or otherwise overcomes an aberrant protein activity present in the cell of the subject.

The therapeutic and/or prophylactic compositions described herein may be administered to a subject using any reasonable amount and any route of administration effective for preventing, treating, diagnosing, or imaging a disease, disorder, and/or condition and/or any other purpose. The specific amount administered to a given subject may vary depending on the species, age, and general condition of the subject, the purpose of the administration, the particular composition, the mode of administration, and the like. Compositions in accordance with the present disclosure may be formulated in dosage unit form for ease of administration and uniformity of dosage. It will be understood, however, that the total daily usage of a composition of the present disclosure will be decided by an attending physician within the scope of sound medical judgment.

A LNP composition including one or more mRNAs may be administered by a variety of routes, for example, orally, intravenously, intramuscularly, intra-arterially, intramedullary, intrathecally, subcutaneously, intraventricularly, trans- or intra-dermally, intradermally, rectally, intravaginally, intraperitoneally, topically, mucosally, nasally, intratumorally. In certain embodiments, an LNP composition may be administered intravenously, intramuscularly, intradermally, intra-arterially, intratumorally, or subcutaneously. However, the present disclosure encompasses the delivery of LNP compositions of the invention by any appropriate route taking into consideration likely advances in the sciences of drug delivery. In general, the most appropriate route of administration will depend upon a variety of factors including the nature of the LNP composition including one or more mRNAs (e.g., its stability in various bodily environments such as the bloodstream and gastrointestinal tract), the condition of the patient (e.g., whether the patient is able to tolerate particular routes of administration), etc.

LNP compositions including one or more mRNAs may be used in combination with one or more other therapeutic, prophylactic, diagnostic, or imaging agents. By “in combination with,” it is not intended to imply that the agents must be administered at the same time and/or formulated for delivery together, although these methods of delivery are within the scope of the present disclosure. For example, one or more LNP compositions including one or more different m RNAs may be administered in combination. Compositions can be administered concurrently with, prior to, or subsequent to, one or more other desired therapeutics or medical procedures. In general, each agent will be administered at a dose and/or on a time schedule determined for that agent. In some embodiments, the present disclosure encompasses the delivery of compositions of the invention, or imaging, diagnostic, or prophylactic compositions thereof in combination with agents that improve their bioavailability, reduce and/or modify their metabolism, inhibit their excretion, and/or modify their distribution within the body.

It will further be appreciated that therapeutically, prophylactically, diagnostically, or imaging active agents utilized in combination may be administered together in a single composition or administered separately in different compositions. In general, it is expected that agents utilized in combination will be utilized at levels that do not exceed the levels at which they are utilized individually. In some embodiments, the levels utilized in combination may be lower than those utilized individually.

The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, a composition useful for treating cancer may be administered concurrently with a chemotherapeutic agent), or they may achieve different effects (e.g., control of any adverse effects).

In some embodiments, no more than 1%, no more than 2%, no more than 3%, no more than 4%, no more than 5%, no more than 6%, no more than 7%, no more than 8%, no more than 9%, no more than 10%, no more than 15%, no more than 20%, no more than 25%, no more than 30%, no more than 35%, no more than 40%, no more than 45%, or no more than 50% of cells that are not meant to be the destination of the delivery are transfected by the LNP. In some embodiments, the cells that are not meant to be the destination of the delivery are subject's non-immune cells. In some embodiments, the cells that are not meant to be the destination of the delivery are cells not targeted by the method. In some embodiments, the cells that are not meant to be the destination of the delivery are subject's cells not targeted by the method.

In some embodiments, the half-life of the nucleic acid delivered by the LNP described herein to the immune cell or a polypeptide encoded by the nucleic acid delivered by the LNP and expressed in the immune cell is at least 1%, at least 5%, at least 10%, at least 15%, at least 20%, at least 25%, at least 30%, at least 35%, at least 40%, at least 45%, at least 50%, at least 60%, at least 70%, at least 80%, at least 90%, at least 2 times, at least 3 times, at least 4 times, or at least 5 times longer than the half-life of the nucleic acid delivered by a reference LNP to the immune cells or a polypeptide encoded by the nucleic acid delivered by the reference LNP and expressed in the immune cell.

In some embodiments, the composition of the LNP differs from the composition of the reference LNP in the type of ionizable cationic lipid, relative amount of ionizable cationic lipid, length of the lipid anchor in PEG lipid, back bone or head group of the PEG lipid, relative amount of PEG lipid, or type of immune cell targeting group, or any combination thereof. In some embodiments, the composition of the LNP differs from the composition of the reference LNP only in the type of ionizable cationic lipid. In some embodiments, the composition of the LNP differs from the composition of the reference LNP only in the amount of PEG lipid. In some embodiments, the reference LNP comprises cationic Lipid DLin-KC3-DMA, but otherwise as the same as a tested LNP. In some embodiments, the reference LNP comprises cationic Lipid DLin-KC2-DMA, but otherwise as the same as a tested LNP. In some embodiments, the reference LNP comprises cationic Lipid ALC-0315, but otherwise as the same as a tested LNP. In some embodiments, the reference LNP comprises cationic Lipid SM-102, but otherwise as the same as a tested LNP. In some embodiments, PEG lipid is a free PEG lipid.

In some embodiments, at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% of the immune cells are transfected by the LNP. In some embodiments, the immune cells are subject's immune cells. In some embodiments, the immune cells are immune cells targeted by the method. In some embodiments, the immune cells are subject's immune cells targeted by the method.

In some embodiments, the expression level of the nucleic acid delivered by the LNP is at least at least 1%, at least 5%, at least 10%, at least 15%, at least 20%, at least 25%, at least 30%, at least 35%, at least 40%, at least 45%, at least 50%, at least 60%, at least 70%, at least 80%, at least 90%, at least 2 times, at least 3 times, at least 4 times, at least 5 times, at least 6 times, at least 7 times, at least 8 times, at least 9 times, or at least 10 times higher than the expression level of the nucleic acid delivered by a reference LNP. In some embodiments, the expression level is measured and compared with a method described herein. In some embodiments, the expression level is measured by the ratio of cells expressing the encoded polypeptide. In some embodiments, the expression level is measured with FACS. In some embodiments, the expression level is measured by the average amount of the encoded polypeptide expressed in cells. In some embodiments, the expression level is measured as mean fluorescence intensity. In some embodiments, the expression level is measured by the amount of the encoded polypeptide or other materials secreted by cells.

In another aspect, provided herein are methods of targeting the delivery of a nucleic acid to an immune cell of a subject. In some embodiments, the method comprises contacting the immune cell with a lipid nanoparticle (LNP). In some embodiments, the LNP comprises an ionizable cationic lipid. In some embodiments, the LNP comprises a conjugate comprising the compound of the following formula: [Lipid]-[optional linker]-[immune cell targeting group]. In some embodiments, the LNP comprises a sterol or other structural lipid. In some embodiments, the LNP comprises a neutral phospholipid. In some embodiments, the LNP comprises a free Polyethylene glycol (PEG) lipid. In some embodiments, the LNP comprises the nucleic acid.

In some embodiments, an aspect of the disclosure relates to an LNP or a pharmaceutical composition containing thereof, as disclosed herein, for use in a method of targeting the delivery of a nucleic acid to an immune cell of a subject. Such a method may be for the treatment of a disease or disorder as disclosed hereafter. In some embodiments, a method as disclosed herein can comprise contacting in vitro or ex vivo the immune cell of a subject with a lipid nanoparticle (LNP). In some embodiments, the LNP is an LNP as described herein in the present disclosure.

In some embodiments, the LNP provides at least one of the following benefits:
(i) increased specificity of targeted delivery to the immune cell compared to a reference LNP;
(ii) increased half-life of the nucleic acid or a polypeptide encoded by the nucleic acid in the immune cell compared to a reference LNP;
(iii) increased transfection rate compared to a reference LNP; and
(iv) a low level of dye accessible mRNA (<15%) and high RNA encapsulation efficiencies, wherein at least 80% mRNA was recovered in final formulation relative to the total RNA used in LNP batch preparation.

In some aspect, provided are methods of expressing a polypeptide of interest in a targeted immune cell of a subject. In some embodiments, the method comprises contacting the immune cell with a lipid nanoparticle (LNP). In some embodiments, the LNP comprises an ionizable cationic lipid. In some embodiments, the LNP comprises a conjugate comprising the following structure: [Lipid]-[optional linker]-[immune cell targeting group]. In some embodiments, the LNP comprises a sterol or other structural lipid. In some embodiments, the LNP comprises a neutral phospholipid. In some embodiments, the LNP comprises a free Polyethylene glycol (PEG) lipid. In some embodiments, the LNP comprises a nucleic acid encoding the polypeptide. In some embodiments, an aspect of the disclosure relates to an LNP or a pharmaceutical composition containing thereof, as disclosed herein, for use in a method of expressing a polypeptide of interest in a targeted immune cell of a subject. Such a method may be for the treatment of a disease or disorder as disclosed hereafter. In some embodiments, a method as disclosed herein can comprise contacting in vitro or ex vivo the immune cell of a subject with a lipid nanoparticle (LNP).

In some embodiments, the LNP provides at least one of the following benefits:
(i) increased expression level in the immune cell compared to a reference LNP;
(ii) increased specificity of expression in the immune cell compared to a reference LNP;
(iii) increased half-life of the nucleic acid or a polypeptide encoded by the nucleic acid in the immune cell compared to a reference LNP;
(iv) increased transfection rate compared to a reference LNP; and
(v) a low level of dye accessible mRNA (<15%) and high RNA encapsulation efficiencies, wherein at least 80% mRNA was recovered in final formulation relative to the total RNA used in LNP batch preparation.

In some aspects, provided are methods of modulating cellular function of a target immune cell of a subject. In some embodiments, the method comprises administering to the subject a lipid nanoparticle (LNP). In some embodiments, the LNP comprises an ionizable cationic lipid. In some embodiments, the LNP comprises a conjugate comprising the following structure: [Lipid]-[optional linker]-[immune cell targeting group]. In some embodiments, the LNP comprises a sterol or other structural lipid. In some embodiments, the LNP comprises a neutral phospholipid. In some embodiments, the LNP comprises a free Polyethylene glycol (PEG) lipid. In some embodiments, the LNP comprises a nucleic acid encoding a polypeptide for modulating the cellular function of the immune cell. In some embodiments, an aspect of the disclosure relates to an LNP or a pharmaceutical composition containing thereof, as disclosed herein, for use in a method of modulating cellular function of a targeted immune cell of a subject. Such a method may be for the treatment of a disease or disorder as disclosed hereafter. In some embodiments, a method as disclosed herein can comprise contacting in vitro or ex vivo the immune cell of a subject with a lipid nanoparticle (LNP).

In some embodiments, the LNP provides at least one of the following benefits:
(i) increased expression level in the immune cell compared to a reference LNP;
(ii) increased specificity of expression in the immune cell compared to a reference LNP;
(iii) increased half-life of the nucleic acid or a polypeptide encoded by the nucleic acid in the immune cell compared to a reference LNP;
(iv) increased transfection rate compared to a reference LNP;
(v) the LNP can be administered at a lower dose compared to a reference LNP to reach the same biologic effect in the immune cell; and
(vi) a low level of dye accessible mRNA (<15%) and high RNA encapsulation efficiencies, wherein at least 80% mRNA was recovered in final formulation relative to the total RNA used in LNP batch preparation.

In some embodiments, the modulation of cell function comprises reprogramming the immune cells to initiate an immune response. In some embodiments, the modulation of cell function comprises modulating antigen specificity of the immune cell.

In some aspect, provided are methods of treating, ameliorating, or preventing a symptom of a disorder or disease in a subject in need thereof. In some embodiments, the method comprises administering to the subject a lipid nanoparticle (LNP) for delivering a nucleic acid into an immune cell of the subject. In some embodiments, the LNP comprises an ionizable cationic lipid. In some embodiments, the LNP comprises a conjugate comprising the following structure: [Lipid]-[optional linker]-[immune cell targeting group]. In some embodiments, the LNP comprises a sterol or other structural lipid. In some embodiments, the LNP comprises a neutral phospholipid. In some embodiments, the LNP comprises a free Polyethylene glycol (PEG) lipid. In some embodiments, the LNP comprises the nucleic acid.

In some embodiments, the nucleic acid modulates the immune response of the immune cell, therefore to treat or ameliorate the symptom. In some embodiments, an aspect of the disclosure relates to an LNP or a pharmaceutical composition containing thereof, as disclosed herein, for use in a method of treating, ameliorating, or preventing a symptom of a disorder or disease in a subject in need thereof. A disease or disorder may be as disclosed hereafter. In some embodiments, a method as disclosed herein can comprise contacting in vitro or ex vivo the immune cell of a subject with a lipid nanoparticle (LNP).

In some embodiments, the LNP provides at least one of the following benefits:
(i) increased specificity of delivery of the nucleic acid into the immune cell compared to a reference LNP;
(ii) increased half-life of the nucleic acid or a polypeptide encoded by the nucleic acid in the immune cell compared to a reference LNP;
(iii) increased transfection rate compared to a reference LNP;
(iv) the LNP can be administered at a lower dose compared to a reference LNP to reach the same treatment efficacy;
(v) increased level of gain of function by an immune cell compared to a reference LNP; and
(vi) a low level of dye accessible mRNA (<15%) and high RNA encapsulation efficiencies, wherein at least 80% mRNA was recovered in final formulation relative to the total RNA used in LNP batch preparation.

In some embodiments, the disorder is an immune disorder, an inflammatory disorder, or cancer. In some embodiments, the nucleic acid encodes an antigen for use in a therapeutic or prophylactic vaccine for treating or preventing an infection by a pathogen.

In some embodiments, no more than 1%, 2%, 3%, 4%, 5%, 6%, 7%, 8%, 9%, or 10% of non-immune cells are transfected by the LNP. In some embodiments, no more than 1%, 2%, 3%, 4%, 5%, 6%, 7%, 8%, 9%, or 10% of undesired immune cells that are not meant to be the destination of the delivery are transfected by the LNP. In some embodiments, the half-life of the nucleic acid delivered by the LNP to the immune cell or a polypeptide encoded by the nucleic acid delivered by the LNP is at least 5%, 10%, 15%, 20%, 25%, 30%, 40%, 50%, 60%, 70%, 80%, 90%, 100%, 1.5 times, 2 times, 3 times, 4 times, 5 times, 10 times, or longer than the half-life of nucleic acid delivered by a reference LNP to the immune cell or a polypeptide encoded by the nucleic acid delivered by the reference LNP.

In some embodiments, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or more immune cells that are meant to be the destination of the delivery are transfected by the LNP.

In some embodiments, expression level of the nucleic acid delivered by the LNP is at least 5%, at least 10%, at least 10%, at least 10%, at least 10%, at least 10%, at least 10%, at least 10%, at least 10%, at least 10%, at least 10%, at least 10%, at least 10%, at least 10%, at least 10%, 1.5 time, 2 times, 3 times, 4 times, 5 times, 10 times, 15 times, 20 times or more higher than expression level of nucleic acid in the same immune cells delivered by a reference LNP.

In some aspects, provided are methods of targeting the delivery of a nucleic acid to an immune cell of a subject. In some embodiments, the method comprises contacting the immune cell with a lipid nanoparticle (LNP) provided herein. In some embodiments, the method is for targeting NK cells. In some embodiments, the immune cell targeting group binds to CD56. In some embodiments, the method is for targeting both T cells and NK cells simultaneously. In some embodiments, the immune cell targeting group binds to CD7, CD8, or both CD7 and CD8. In some embodiments, the method is for targeting both CD4+ and CD8+ T cells simultaneously. In some embodiments, the immune cell targeting group comprises a polypeptide that binds to CD3 or CD7.

In some aspects, provided are methods of expressing a polypeptide of interest in a targeted immune cell of a subject. In some embodiments, the method comprises contacting the immune cell with a lipid nanoparticle (LNP) provided herein.

In some aspect, provided are method of modulating cellular function of a target immune cell of a subject. In some embodiments, the method comprises administering to the subject a lipid nanoparticle (LNP) provided herein.

In some aspects, provided are method of treating, ameliorating, or preventing a symptom of a disorder or disease in a subject in need thereof. In some embodiments, the method comprises administering to the subject a lipid nanoparticle (LNP) provided herein.

In some aspects, provided are methods of treating a disease or disorder related to CD8 in a subject. In some embodiments, the method comprises administering a pharmaceutical composition described herein to the subject. In some embodiments, the disease or disorder is cancer.

LNPs disclosed in the present disclosure and as claimed are suitable for the methods described above.

### VIII. Kits for Use in Medical Applications

Another aspect of the invention provides a kit for treating a disorder. The kit comprises: an ionizable cationic lipid, a lipid-immune cell targeting group conjugate, a lipid nanoparticle composition comprising an ionizable cationic lipid and/or a lipid-immune cell targeting group conjugate with or without an encapsulated payload (e.g., an mRNA), and instructions for treating a medical disorder, such as, cancer or a microbial or viral infection.

### ENUMERATED EMBODIMENTS

The following enumerated embodiments are representative of some aspects of the invention.

1. A compound of Formula (I):

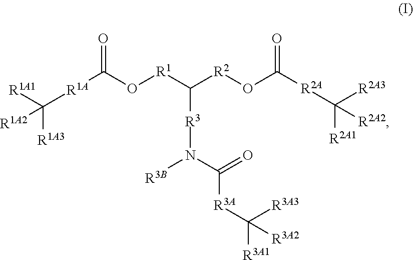

or a salt thereof, wherein:
R¹, R², and R³ are each independently a bond or C₁₋₃ alkylene;
R^{1A}, R^{2A}, and R^{3A} are each independently a bond or C₁₋₁₀ alkylene;
R^{1A1}, R^{1A2}, R^{1A3}, R^{2A1}, R^{2A2}, R^{2A3}, R^{3A1}, R^{3A2}, and R^{3A3} are each independently H, C₁₋₂₀ alkyl, C₁₋₂₀ alkenyl, —(CH₂)₀₋₁₀C(O)OR^{a1}, or —(CH₂)₀₋₁₀OC(O)R^{a2};
R^{a1} and R^{a2} are each independently C₁₋₂₀ alkyl or C₁₋₂₀ alkenyl;
R^{3B} is

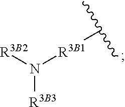

R^{3B1} is C₁₋₆ alkylene; and
R^{3B2} and R^{3B3} are each independently H or C₁₋₆ alkyl.
   2. The compound of embodiment 1, or a salt thereof, wherein the compound is a compound of Formula (Ia):

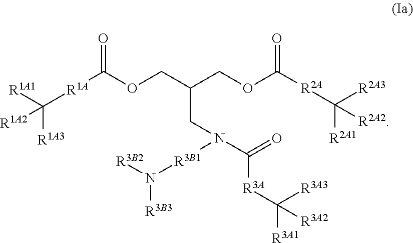

3. The compound of embodiment 1 or 2, or a salt thereof, wherein R^{3B1} is ethylene or propylene.
4. The compound of any one of embodiments 1 to 3, or a salt thereof, wherein R^{3B2} and R^{3B3} are each independently H or C₁₋₆ alkyl optionally substituted with one or more substituents each independently selected from the group consisting of —OH and —O—(C₁₋₆ alkyl).
5. The compound of embodiment 4, or a salt thereof, wherein R^{3B2} and R^{3B3} are each independently methyl or ethyl, each optionally substituted with one or more —OH.
6. The compound of embodiment 5, or a salt thereof, wherein R^{3B2} and R^{3B3} are each unsubstituted methyl.
7. The compound of embodiment 1 or 2, or a salt thereof, wherein R^{3B3} is

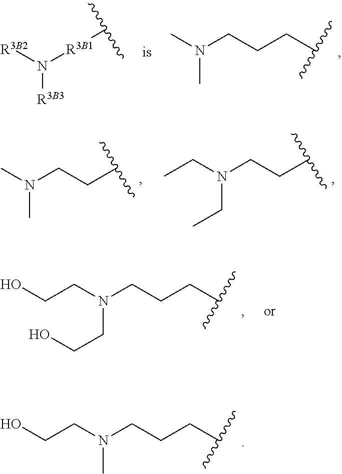

8. The compound of any one of embodiments 1 to 7, or a salt thereof, wherein R¹, R², and R³ are each independently a bond or methylene.
9. The compound of embodiment 8, or a salt thereof, wherein R¹ and R² are each methylene and R³ is a bond.
10. The compound of embodiment 8, or a salt thereof, wherein R¹, R², and R³ are each methylene.
11. The compound of embodiment 1, or a salt thereof, wherein the compound is a compound of Formula (Ib):

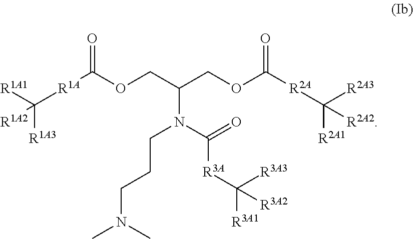

12. The compound of any one of embodiments 1 to 11, or a salt thereof, wherein R^{1A}R^{2A}, and R^{3A} are each independently a bond or —(CH₂)₁₋₁₀—.
13. The compound of embodiment 12, or a salt thereof, wherein R^{1A} and R^{2A} are each independently a bond, —CH₂—, —(CH₂)₂—, —(CH₂)₃—, —(CH₂)₄—, —(CH₂)₅—, —(CH₂)₆—, —(CH₂)₇—, or —(CH₂)₈—.
14. The compound of embodiment 13, or a salt thereof, wherein R^{1A} and R^{2A} are each independently a bond, —(CH₂)₂—, —(CH₂)₄—, —(CH₂)₆—, —(CH₂)₇—, or —(CH₂)₈—.
15. The compound of any one of embodiments 12 to 14, or a salt thereof, wherein R^{3A} is a bond, —CH₂—, —(CH₂)₂—, or —(CH₂)₇—.
16. The compound of any one of embodiments 1 to 15, or a salt thereof, wherein R^{1A1}R^{1A2}, R^{1A3}, R^{2A1}, R^{2A2}, and R^{2A3} are each independently H, C₁₋₁₅ alkyl, —CH═CH—(C₁₋₁₅ alkyl), —CH═CH—CH₂—CH═CH—(C₁₋₁₀ alkyl), —(CH₂)₀₋₄C(O)OCH(C₁₋₁₀ alkyl)(C₁₋₁₅ alkyl), —(CH₂)₀₋₄OC(O)CH(C₁₋₁₀ alkyl)(C₁₋₁₅ alkyl), —(CH₂)₀₋₄C(O)OCH₂(C₁₋₁₅ alkyl), or —(CH₂)₀₋₄OC(O)CH₂(C₁₋₁₅ alkyl).
17. The compound of embodiment 16, or a salt thereof, wherein R^{1A1} and R^{2A1} are each independently —CH═CH—(C₁₋₁₅ alkyl), —CH═CH—CH₂—CH═CH—(C₁₋₁₀ alkyl), —(CH₂)₀₋₄C(O)OCH(C₁₋₁₀ alkyl)(C₁₋₁₅ alkyl), or —(CH₂)₀₋₄OC(O)CH(C₁₋₁₀ alkyl)(C₁₋₁₅ alkyl); and R^{1A2}, R^{1A3}, R^{2A2}, and R^{2A3} are each H.
18. The compound of embodiment 17, or a salt thereof, wherein R^{1A1} and R^{2A1} are each

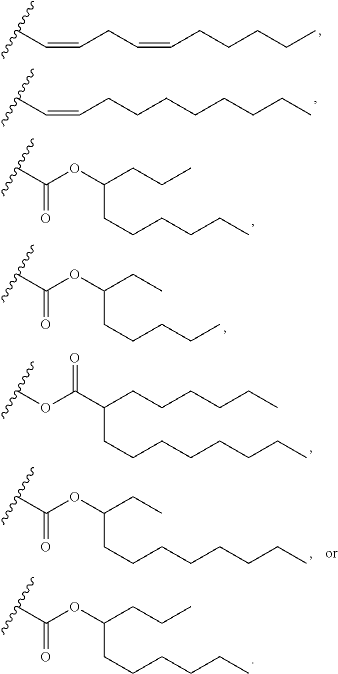

19. The compound of embodiment 16, or a salt thereof, wherein R^{1A1} and R^{2A1} are each C₁₋₁₅ alkyl; R^{1A2} and R^{2A2} are each C₁₋₁₅ alkyl; and R^{1A3} and R^{2A3} are each H.
20. The compound of embodiment 19, or a salt thereof, wherein R^{1A1} and R^{2A1} are each

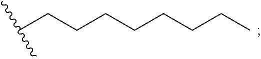 [SMILES: *CCCCCCCC]

and R^{1A2} and R^{2A2} are each

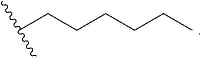 [SMILES: *CCCCCC]

21. The compound of embodiment 16, or a salt thereof, wherein R^{1A1} and R^{2A1} are each —(CH₂)₀₋₄OC(O)CH₂(C₁₋₁₅ alkyl); R^{2A1} and R^{2A2} are each —(CH₂)₀₋₄C(O)OCH₂(C₁₋₁₅ alkyl); and R^{1A3} and R^{2A3} are each H.
22. The compound of embodiment 21, or a salt thereof, wherein R^{1A1} and R^{2A1} are each

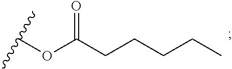 [SMILES: *OC(=O)CCCCC]

and R^{2A1} and R^{2A2} are each

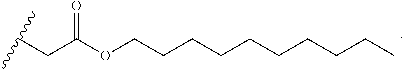 [SMILES: *CC(=O)OCCCCCCCCCC]

23. The compound of embodiment 16, or a salt thereof, wherein R^{1A1} and R^{2A1} are each —C(O)OCH₂(C₁₋₁₅ alkyl); R^{1A2} and R^{2A2} are each —(CH₂)₀₋₄C(O)OCH₂(C₁₋₁₅ alkyl); and R^{1A3} and R^{2A3} are each H.
24. The compound of embodiment 23, or a salt thereof, wherein R^{1A1} and R^{2A1} are each

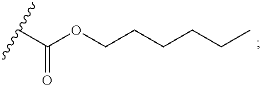 [SMILES: *C(=O)OCCCCCC]

and R^{1A2} and R^{2A2} are each

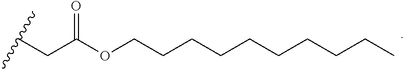 [SMILES: *CC(=O)OCCCCCCCCCC]

25. The compound of any one of embodiments 1 to 24, or a salt thereof, wherein R^{3A1}, R^{3A2}, and R^{3A3} are each independently H, C₁₋₁₅ alkyl, —(CH₂)₀₋₄C(O)OCH(C₁₋₅ alkyl)(C₁₋₁₀ alkyl), —(CH₂)₀₋₄OC(O)CH(C₁₋₅ alkyl)(C₁₋₁₀ alkyl), —(CH₂)₀₋₄C(O)OCH₂(C₁₋₁₀ alkyl), or —(CH₂)₀₋₄OC(O)CH₂(C₁₋₁₀ alkyl).
26. The compound of embodiment 25, or a salt thereof, wherein R^{3A1} and R^{3A2} are each independently C₁₋₁₅ alkyl; and R^{3A3} is H.
27. The compound of embodiment 26, or a salt thereof, wherein R^{3A1} and R^{3A2} are each independently ethyl,

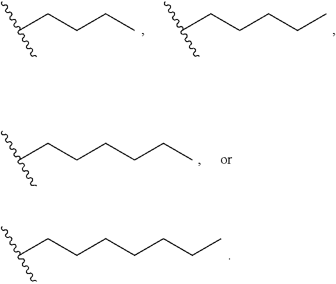

28. The compound of embodiment 25, or a salt thereof, wherein R^{3A1} is C₁₋₁₅ alkyl; and R^{3A2} and R^{3A3} are each H.
29. The compound of embodiment 28, or a salt thereof, wherein R^{3A1} is

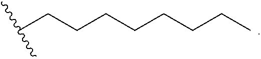 [SMILES: *CCCCCCCC]

30. The compound of embodiment 25, or a salt thereof, wherein R^{3A1} is —C(O)OCH(C₁₋₅ alkyl)(C₁₋₁₀ alkyl); and R^{3A2} and R^{3A3} are each H.
31. The compound of embodiment 30, or a salt thereof, wherein R^{3A1} is

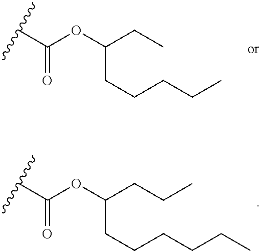 [SMILES: *C(=O)OC(CC)CCCCC ; *C(=O)OC(CCC)CCCCCC]

32. The compound of embodiment 25, or a salt thereof, wherein R^{3A1} is —(CH₂)₀₋₄OC(O)CH₂(C₁₋₁₀ alkyl); R^{3A2} is —(CH₂)₀₋₄(O)OCH₂(C₁₋₁₀ alkyl); and R^{3A3} is H.
33. The compound of embodiment 32, or a salt thereof, wherein R^{3A1} is

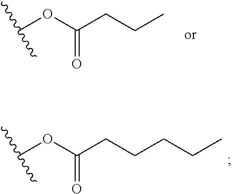

### and R^{3A2} is

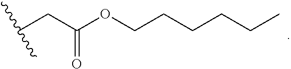 [SMILES: *CC(=O)OCCCCCC]

34. The compound of embodiment 25, or a salt thereof, wherein R^{3A1} is —(CH₂)₀₋₄C(O)OCH₂(C₁₋₁₀ alkyl); R^{3A2} is —(CH₂)₀₋₄C(O)OCH₂(C₁₋₁₀ alkyl); and R^{3A3} is H.
35. The compound of embodiment 34, or a salt thereof, wherein R^{3A1} is

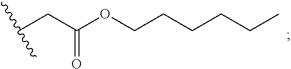 [SMILES: *CC(=O)OCCCCCC]

### and R^{3A2} is

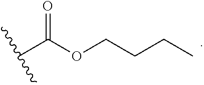 [SMILES: *C(=O)OCCCC]

36. The compound of embodiment 25, or a salt thereof, wherein R^{3A1}, R^{3A2}, and R^{3A3} are each H.
37. The compound of any one of embodiments 1 to 15, or a salt thereof, wherein R^{a1} and R^{a2} are each independently —(CH₂)₀₋₁₅CH₃ or —CH(C₁₋₁₀ alkyl)(C₁₋₁₅ alkyl).
38. The compound of embodiment 37, or a salt thereof, wherein R^{a1} and R^{a2} are each independently

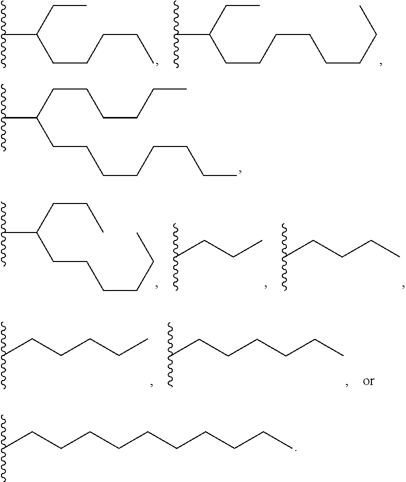

39. The compound of embodiment 1, or a salt thereof, wherein the compound is selected from Table 1.
40. The compound of embodiment 1, or a salt thereof, wherein the compound is

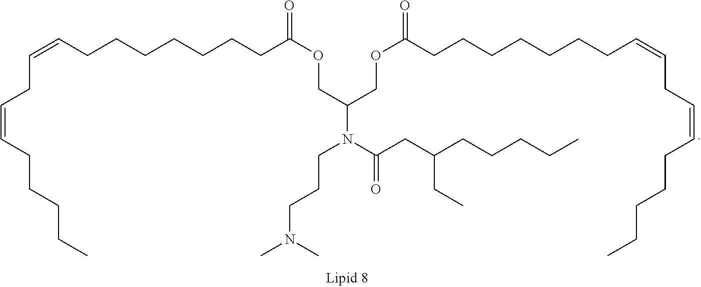 [SMILES: CCCCC/C=C\C/C=C\CCCCCCCC(=O)OCC(COC(=O)CCCCCCC/C=C\C/C=C\CCCCC)N(CCCN(C)C)C(=O)CC(CC)CCCCC]

41. The compound of embodiment 1, or a salt thereof, wherein the compound is

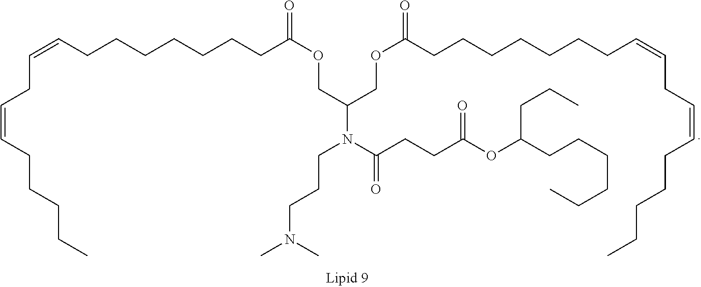 [SMILES: CCCCC/C=C\C/C=C\CCCCCCCC(=O)OCC(COC(=O)CCCCCCC/C=C\C/C=C\CCCCC)N(CCCN(C)C)C(=O)CCC(=O)OC(CCC)CCCCCC]

42. The compound of embodiment 1, or a salt thereof, wherein the compound is

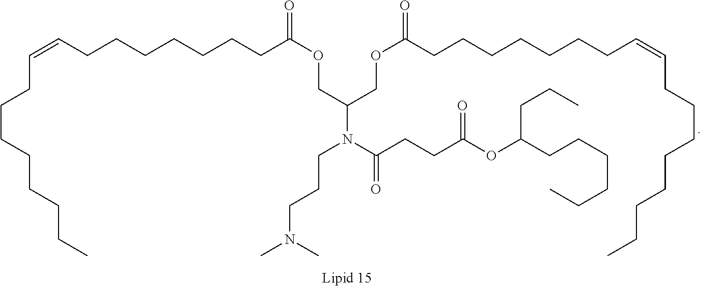 [SMILES: CCCCCCCC/C=C\CCCCCCCC(=O)OCC(COC(=O)CCCCCCC/C=C\CCCCCCCC)N(CCCN(C)C)C(=O)CCC(=O)OC(CCC)CCCCCC]

43. A lipid nanoparticle (LNP) comprising a lipid blend for targeted delivery of a nucleic acid into an immune cell, the lipid blend comprising:
(a) a lipid-immune cell targeting group conjugate comprising the compound of Formula (II): [Lipid]-[optional linker]-[immune cell targeting group], and
(b) an ionizable cationic lipid comprising any one of the compound of embodiments 1 to 42,
wherein the LNP further comprises a nucleic acid disposed therein.
   44. The LNP of embodiment 43, wherein the immune cell targeting group comprises an antibody that binds a T cell antigen.
   45. The LNP of embodiment 44, wherein the T cell antigen is CD3, CD4, CD7, CD8, or a combination thereof (e.g., both CD3 and CD8, both CD4 and CD8, or both CD7 and CD8).
   46. The LNP of any one of embodiments 43 to 45, wherein the immune cell targeting group comprises an antibody that binds a Natural Killer (NK) cell antigen.
   47. The LNP of embodiment 46, wherein the NK cell antigen is CD7, CD8, CD56, or a combination thereof (e.g., both CD7 and CD8).
   48. The LNP of any one of embodiments 43 to 47, wherein the immune cell targeting group is covalently coupled to a lipid in the lipid blend via a polyethylene glycol (PEG) containing linker.
   49. The LNP of embodiment 48, wherein the lipid covalently coupled to the immune cell targeting group via a PEG containing linker is distearoylglycerol (DSG), distearoyl-phosphatidylethanolamine (DSPE), dimyrstoyl-phosphatidylethanolamine (DMPE), distearoyl-glycero-phosphoglycerol (DSPG), dimyristoyl-glycerol (DMG), dipalmitoyl-phosphatidylethanolamine (DPPE), dipalmitoyl-glycerol (DPG), or ceramide.
   50. The LNP of embodiment 48 or 49, wherein the PEG is PEG 2000 or PEG 3400.
   51. The LNP of any one of embodiments 43 to 50, wherein the lipid-immune cell targeting group conjugate is present in the lipid blend in a range of 0.001 to 0.5 mole percent (e.g., 0.002-0.2 mole percent).
   52. The LNP of any one of embodiments 43 to 51, wherein the lipid blend further comprises one or more of a structural lipid (e.g., a sterol), a neutral phospholipid, and a free PEG-lipid.
   53. The LNP of any one of embodiments 43 to 52, wherein the ionizable cationic lipid is present in the lipid blend in a range of 30-70 (e.g., 40-60) mole percent.
   54. The LNP of embodiment 52, wherein the sterol is present in the lipid blend in a range of 20-70 (e.g., 30-50) mole percent.
   55. The LNP of embodiment 52 or 54, wherein the sterol is cholesterol.
   56. The LNP of any one of embodiments 52 to 55, wherein the neutral phospholipid is selected from the group consisting of phosphatidylcholine, phosphatidylethanolamine, distearoyl-sn-glycero-3-phosphoethanolamine (DSPE), 1,2-distearoyl-sn-glycero-3-phosphocholine (DSPC), 1,2-dioleoyl-sn-glycero-3-phosphoethanolamine (DOPE), 1,2-dioleoyl-sn-glycero-3-phosphocholine (DOPC), and sphingomyelin.
   57. The LNP of any one of embodiments 52 to 56, wherein the neutral phospholipid is present in the lipid blend in a range of 5-15 mole percent.
   58. The LNP of any one of embodiments 52 to 57, wherein the free PEG-lipid is selected from the group consisting of PEG-modified phosphatidylethanolamines, PEG-modified phosphatidic acids, PEG-modified ceramides, PEG-modified dialkylamines, PEG-modified diacylglycerols, and PEG-modified dialkylglycerols. For example, a PEG lipid may be PEG-dioleoylgylcerol (PEG-DOG), PEG-dimyristoyl-glycerol (PEG-DMG), PEG-dipalmitoyl-glycerol (PEG-DPG), PEG-dilinoleoyl-glycero-phosphatidyl ethanolamine (PEG-DLPE), PEG-dimyrstoyl-phosphatidylethanolamine (PEG-DMPE), PEG-dipalmitoyl-phosphatidylethanolamine (PEG-DPPE), PEG-distearoylglycerol (PEG-DSG), PEG-diacylglycerol (PEG-DAG, e.g., PEG-DMG, PEG-DPG, and PEG-DSG), PEG-ceramide, PEG-distearoyl-glycero-phosphoglycerol (PEG-DSPG), PEG-dioleoyl-glycero-phosphoethanolamine (PEG-DOPE), 2-[(polyethylene glycol)-2000]-N,N-ditetradecylacetamide, or a PEG-distearoyl-phosphatidylethanolamine (PEG-DSPE) lipid.
   59. The LNP of any one of embodiments 52 to 57, wherein the free PEG-lipid comprises a diacylphosphatidylethanolamine comprising Dipalmitoyl (C16) chain or Distearoyl (C18) chain, and optionally the free PEG-lipid comprises PEG-DPG and PEG-DMG.
   60. The LNP of any one of embodiments 52 to 59, wherein the free PEG-lipid is present in the lipid blend in a range of 1-4 mole percent.
   61. The LNP of any one of embodiments 52 to 60, wherein the free PEG-lipid comprises the same or a different lipid as the lipid in the lipid-immune cell targeting group conjugate.
   62. The LNP of any one of embodiments 43 to 61, wherein the LNP has a mean diameter in the range of 50-200 nm.
   63. The LNP of embodiment 62, where the LNP has a mean diameter of about 100 nm.
   64. The LNP of any one of embodiments 43 to 63, wherein the LNP has a polydispersity index in a range from 0.05 to 1.
   65. The LNP of any one of embodiments 43 to 64, wherein the LNP has a zeta potential of from about +10 mV to about +30 mV at pH 5.
   66. The LNP of any one of embodiments 43 to 65, wherein the nucleic acid is DNA or RNA. 67. The LNP of embodiment 66, wherein the RNA is an mRNA.
   68. The LNP of embodiment 67, wherein the mRNA encodes a receptor, a growth factor, a hormone, a cytokine, an antibody, an antigen, an enzyme, or a vaccine.
   69. The LNP of embodiment 67, wherein the mRNA encodes a polypeptide capable of regulating immune response in the immune cell.
   70. The LNP of embodiment 67, wherein the mRNA encodes a polypeptide capable of reprogramming the immune cell.
   71. The LNP of embodiment 69, wherein the mRNA encodes a synthetic T cell receptor (synTCR) or a Chimeric Antigen Receptor (CAR).
   72. The LNP of any one of embodiments 43 to 71, wherein the immune cell targeting group comprises an antibody, and the antibody is a Fab or an immunoglobulin single variable domain (e.g., a Nanobody).
   73. The LNP of any one of embodiments 43 to 71, wherein the immune cell targeting group comprises a Fab, F(ab′)2, Fab′-SH, Fv, or scFv fragment.
   74. The LNP of embodiment 72 or embodiment 73, wherein the immune cell targeting group comprises a Fab that is engineered to knock out the natural interchain disulfide bond at the C-terminus.
   75. The LNP of embodiment 74, wherein the Fab comprises a heavy chain fragment that comprises C233S substitution, and a light chain fragment that comprises C214S substitution, numbering according to Kabat.
   76. The LNP of any one of embodiments 73 to 75, wherein the immune cell targeting group comprises a Fab that has a non-natural interchain disulfide bond (e.g., an engineered, buried interchain disulfide bond).
   77. The LNP of embodiment 76, wherein the Fab comprises F174C substitution in the heavy chain fragment, and S176C substitution in the light chain fragment, numbering according to Kabat.
   78. The LNP of embodiments 73 to 77, wherein the immune cell targeting group comprises a Fab that comprises a cysteine at the C-terminus of the heavy or light chain fragment.
   79. The LNP of embodiment 78, wherein the Fab further comprises one or more amino acids between the heavy chain fragment of the Fab and the C-terminal cysteine.
   80. The LNP of embodiment 72, wherein the immune cell targeting group comprises an immunoglobulin single variable domain.
   81. The LNP of embodiment 72 or 80, wherein the immunoglobulin single variable domain comprises a cysteine at the C-terminus.
   82. The LNP of embodiment 81, wherein the immunoglobulin single variable domain comprises a VHH domain and further comprises a spacer comprising one or more amino acids between the VHH domain and the C-terminal cysteine.
   83. The LNP of any one of embodiments 73 and 80 to 82, wherein the immune cell targeting group comprises two or more V_{HH} domains.
   84. The LNP of embodiment 83, wherein the two or more V_{HH} domains are linked by an amino acid linker.
   85. The LNP of embodiment 83, wherein the immune cell targeting group comprises a first V_{HH} domain linked to an antibody CH1 domain and a second V_{HH} domain linked to an antibody light chain constant domain, and wherein the antibody CH1 domain and the antibody light chain constant domain are linked by one or more disulfide bonds.
   86. The LNP of any one of embodiments 72 and 80 to 82, wherein the immune cell targeting group comprises a V_{HH} domain linked to an antibody CH1 domain, and wherein the antibody CH1 domain is linked to an antibody light chain constant domain by one or more disulfide bonds.
   87. The LNP of embodiment 85 or 86, wherein the CH1 domain comprises F174C and C233S substitutions, and the light chain constant domain comprises S176C and C214S substitutions, numbering according to Kabat.
   88. The LNP of any one of embodiments 43 to 69, wherein the immune cell targeting group comprises a Fab that comprises:
(a) a heavy chain fragment comprising the amino acid sequence of SEQ ID NO: 1 and a light chain fragment comprising the amino acid sequence of SEQ ID NO:2 or 3; or
(b) a heavy chain fragment comprising the amino acid sequence of SEQ ID NO: 6 and a light chain fragment comprising the amino acid sequence of SEQ ID NO: 7.
   89. The LNP of any one of embodiments 43 to 88, wherein the ionizable cationic lipid is

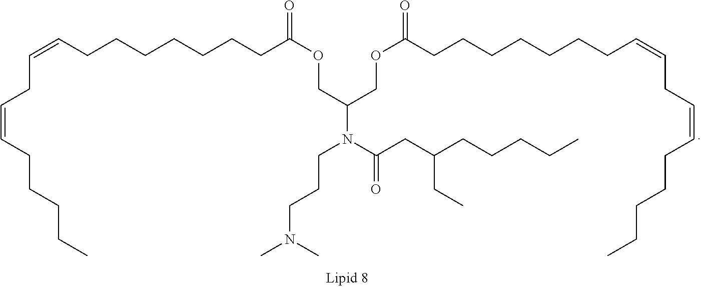 [SMILES: CCCCC/C=C\C/C=C\CCCCCCCC(=O)OCC(COC(=O)CCCCCCC/C=C\C/C=C\CCCCC)N(CCCN(C)C)C(=O)CC(CC)CCCCC]

90. The LNP of any one of embodiments 43 to 88, wherein the ionizable cationic lipid is

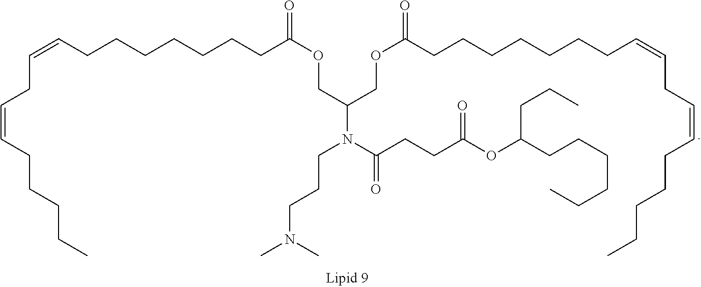 [SMILES: CCCCC/C=C\C/C=C\CCCCCCCC(=O)OCC(COC(=O)CCCCCCC/C=C\C/C=C\CCCCC)N(CCCN(C)C)C(=O)CCC(=O)OC(CCC)CCCCCC]

91. The LNP of any one of embodiments 43 to 88, wherein the ionizable cationic lipid is

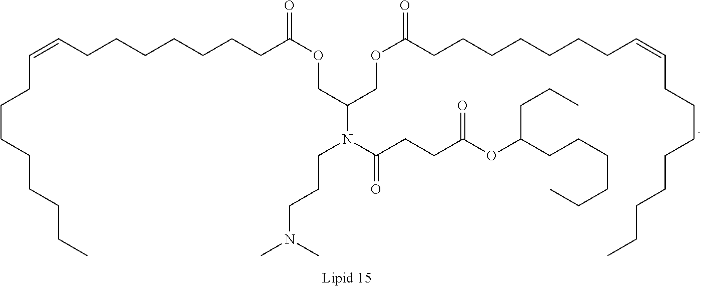 [SMILES: CCCCCCCC/C=C\CCCCCCCC(=O)OCC(COC(=O)CCCCCCC/C=C\CCCCCCCC)N(CCCN(C)C)C(=O)CCC(=O)OC(CCC)CCCCCC]

92. The LNP of any one of embodiments 43 to 91, wherein the LNP comprises:
(a) the ionizable cationic lipid,
(b) the conjugate comprising the compound of the following formula:

[Lipid]-[optional linker]-[immune cell targeting group];
(c) a sterol or other structural lipid;
(d) a neutral phospholipid
(e) a free Polyethylene glycol (PEG) lipid, and
(f) the nucleic acid.
   93. The LNP of any one of embodiments 43 to 92, wherein the LNP is for delivering a nucleic acid into an immune cell, and wherein the immune cell is an NK cell, and the immune cell targeting group comprises an antibody that binds CD56.
   94. The LNP of any one of embodiments 43 to 92, wherein the LNP is for delivering a nucleic acid into an immune cell, and wherein the immune cell targeting group comprises an antibody that binds CD7 or CD8, and the free PEG lipid is DMG-PEG or PEG-DPG.
   95. The LNP of any one of embodiments 43 to 92, wherein the immune cell targeting group comprises an antibody, and the antibody is a Fab or an immunoglobulin single variable domain.
   96. The LNP of embodiment 95, wherein the Fab is engineered to knock out the natural interchain disulfide at the C-terminus.
   97. The LNP of embodiment 96, wherein the Fab comprises a heavy chain fragment that comprises C233S substitutions, and a light chain fragment that comprises C214S substitutions.
   98. The LNP of embodiment 96, wherein the Fab comprises a non-natural interchain disulfide.
   99. The LNP of embodiment 96, wherein the Fab comprises F174C substitution in the heavy chain fragment, and S176C substitution in the light chain fragment.
   100. The LNP of embodiment 95, wherein the antibody is an immunoglobulin single variable (ISV) domain, and the ISV domain is an Nanobody® ISV.
   101. The LNP of embodiment 100, wherein the free PEG lipid comprises a PEG having a molecular weight of at least 2000 daltons.
   102. The LNP of embodiment 101, wherein the PEG has a molecular weight of about 3000 to 5000 daltons.
   103. The LNP of embodiment 95, wherein the antibody is a Fab.
   104. The LNP of embodiment 103, wherein the Fab binds CD3, and the free PEG lipid in the LNP comprises a PEG having a molecular weight of about 2000 daltons.
   105. The LNP of embodiment 103, wherein the Fab is an anti-CD4 antibody, and the free PEG lipid in the LNP comprises a PEG having a molecular weight of about 3000 to 3500 daltons.
   106. The LNP of embodiment 95, wherein the immune cell targeting group comprises two or more V_{HH} domains.
   107. The LNP of embodiment 106, wherein the two or more V_{HH} domains are linked by an amino acid linker.
   108. The LNP of embodiment 107, wherein the immune cell targeting group comprises a first V_{HH} domain linked to an antibody CH1 domain and a second V_{HH} domain linked to an antibody light chain constant domain.
   109. The LNP of any one of embodiments 43 to 92, wherein the LNP is for delivering a nucleic acid into an immune cell, and wherein the LNP binds CD3, and also binds CD11a or CD18.
   110. The LNP of embodiment 109, wherein the LNP comprises two conjugates, wherein the first conjugate comprises an antibody that binds CD3, and the second conjugate comprises an antibody that binds CD11a or CD18.
   111. The LNP of embodiment 109, wherein the LNP comprises one conjugate, and the conjugate comprises a bispecific antibody that binds both CD3 and CD11a.
   112. The LNP of embodiment 109, wherein the LNP comprises one conjugate, and the conjugate comprises a bispecific antibody that binds both CD3 and CD18.
   113. The LNP of embodiment 111 or 112, wherein the bispecific antibody is an immunoglobulin single variable domain or Fab-ScFv.
   114. The LNP of any one of embodiments 43 to 92, wherein the LNP is for delivering a nucleic acid into an immune cell, and wherein the LNP binds CD7 and CD8 of the immune cell.
   115. The LNP of embodiment 114, wherein the LNP comprises two conjugates, wherein the first conjugate comprises an antibody that binds CD7, and a second conjugate that binds CD8.
   116. The LNP of embodiment 114, wherein the LNP comprises one conjugate, wherein the conjugate comprises a bispecific antibody that binds CD7 and CD8.
   117. The LNP of embodiment 116, wherein the bispecific antibody is an immunoglobulin single variable domain or Fab-ScFv.
   118. The LNP of any one of embodiments 43 to 92, wherein the LNP binds to a first antigen on the surface of the first type of immune cell, and also binds to a second antigen on the surface of the second type of immune cell.
   119. The LNP of embodiment 118, wherein the two different types of immune cells are CD4+ T cells and CD8+ T cell.
   120. The LNP of embodiment 118, wherein the LNP comprises two conjugates, and the first conjugate comprises a first antibody that binds to the first antigen of the first type of immune cell, and the second conjugate comprises a second antibody that binds to the second antigen of the second type of immune cell.
   121. The LNP of embodiment 118, wherein the LNP comprises one conjugate, and the conjugate comprises a bispecific antibody, and the bispecific antibody binds to both the first antigen on the first type of immune cell, and the second antigen on the second type of immune cells.
   122. The LNP of any one of embodiments 43 to 92, wherein the bispecific antibody is an immunoglobulin single variable domain or a Fab-ScFv.
   123. The LNP of any one of embodiments 43 to 92, wherein the LNP is for delivering a nucleic acid into an immune cell, and wherein the immune cell targeting group comprises a single antibody that binds to CD3 or CD7.
   124. The LNP of any one of embodiments 43 to 92, wherein the LNP is for delivering a nucleic acid into an immune cell, and wherein the immune cell targeting group binds to CD7, CD8, or both CD7 and CD8.
   125. The LNP of any one of embodiments 43 to 92, wherein the LNP is for delivering a nucleic acid into both T cells and NK cells, wherein the immune cell targeting group binds to
(a) both CD3 and CD56;
(b) both CD8 and CD56; or
(c) both CD7 and CD56.
   126. The LNP of any one of embodiments 93 to 125, wherein the LNP has a mean diameter in the range of 50-200 nm.
   127. The LNP of embodiment 126, where the LNP has a mean diameter of about 100 nm.
   128. The LNP of any one of embodiments 93 to 125, wherein the LNP has a polydispersity index in a range from 0.05 to 1.
   129. The LNP of any one of embodiments 93 to 128, wherein the LNP has a zeta potential of from about +10 mV to about +30 mV at pH 5.
   130. The LNP of any one of embodiments 93 to 129, wherein the nucleic acid is DNA or RNA.
   131. The LNP of embodiment 130, wherein the RNA is an mRNA.
   132. The LNP of embodiment 131, wherein the mRNA encodes a receptor, a growth factor, a hormone, a cytokine, an antibody, an antigen, an enzyme, or a vaccine.
   133. The LNP of embodiment 131, wherein the mRNA encodes a polypeptide capable of regulating immune response in the immune cell.
   134. The LNP of embodiment 133, wherein the mRNA encodes a polypeptide capable of reprogramming the immune cell.
   135. The LNP of embodiment 134, wherein the mRNA encodes a synthetic T cell receptor (synTCR) or a Chimeric Antigen Receptor (CAR).
   136. The LNP of any one of embodiments 43 to 92, wherein the LNP is for delivering a nucleic acid into an immune cell, and wherein the immune cell targeting group comprises a Fab lacking the native interchain disulfide bond.
   137. The LNP of embodiment 136, wherein the Fab is engineered to replace one or both cysteines on the native constant light chain and the native constant heavy chain that form the native interchain disulfide with a non-cysteine amino acid, therefor to remove the native interchain disulfide bond in the Fab.
   138. A method of targeting the delivery of a nucleic acid to an immune cell of a subject, comprising contacting the immune cell with the LNP of any one of embodiments 43 to 137, wherein the LNP comprises the nucleic acid.
   139. A method of expressing a polypeptide of interest in a targeted immune cell of a subject, comprising contacting the immune cell with the LNP of any one of embodiments 43 to 137, wherein the LNP comprises a nucleic acid encoding the polypeptide.
   140. A method of modulating cellular function of a target immune cell of a subject, comprising administering to the subject the LNP of any one of embodiments 43 to 137, wherein the LNP comprises a nucleic acid modulates the cellular function of the immune cell.
   141. A method of treating, ameliorating, or preventing a symptom of a disorder or disease in a subject in need thereof, comprising administering to the subject an LNP for delivering a nucleic acid into an immune cell of the subject, wherein the LNP is any one of embodiments 43 to 137, wherein the LNP comprises the nucleic acid.
   142. The method of embodiment 141, wherein the disorder is an immune disorder, an inflammatory disorder, or cancer.
   143. The method of embodiment 141, wherein the nucleic acid encodes an antigen for use in a therapeutic or prophylactic vaccine for treating or preventing an infection by a pathogen.
   144. The method of any one of embodiments 138 to 143, wherein the ionizable cationic lipid is

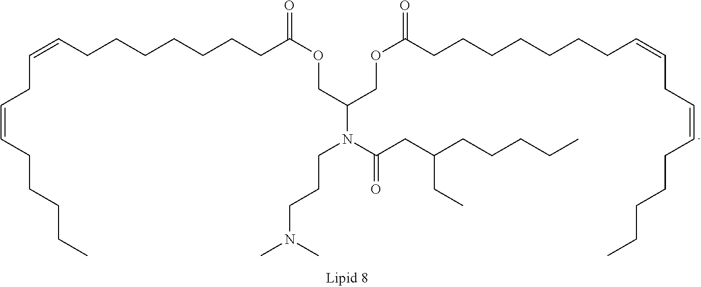 [SMILES: CCCCC/C=C\C/C=C\CCCCCCCC(=O)OCC(COC(=O)CCCCCCC/C=C\C/C=C\CCCCC)N(CCCN(C)C)C(=O)CC(CC)CCCCC]

145. The method of any one of embodiments 138 to 143, wherein the ionizable cationic lipid is

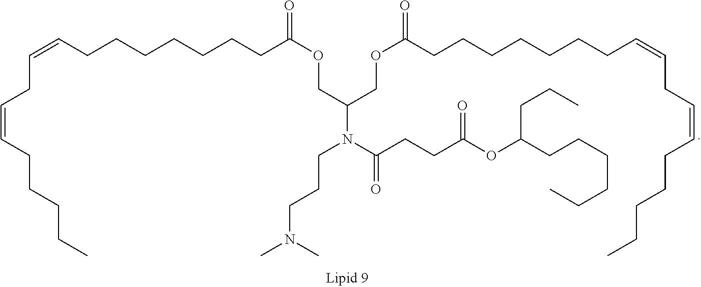 [SMILES: CCCCC/C=C\C/C=C\CCCCCCCC(=O)OCC(COC(=O)CCCCCCC/C=C\C/C=C\CCCCC)N(CCCN(C)C)C(=O)CCC(=O)OC(CCC)CCCCCC]

146. The method of any one of embodiments 138 to 143, wherein the ionizable cationic lipid is

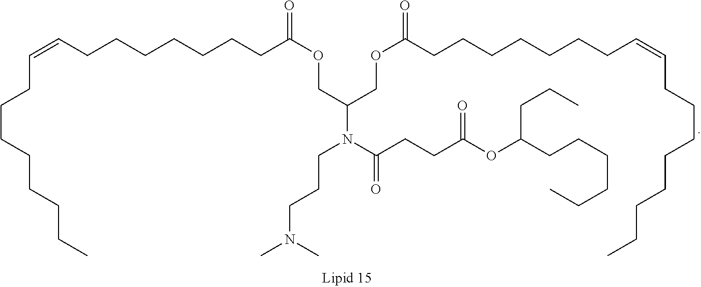 [SMILES: CCCCCCCC/C=C\CCCCCCCC(=O)OCC(COC(=O)CCCCCCC/C=C\CCCCCCCC)N(CCCN(C)C)C(=O)CCC(=O)OC(CCC)CCCCCC]

147. The method of any one of embodiments 138 to 146, wherein the immune cell targeting group comprises an antibody that binds a T cell antigen.
148. The method of embodiment 147, wherein the T cell antigen is CD3, CD8, or both CD3 and CD8.
149. The method of any one of embodiments 138 to 146, wherein the immune cell targeting group comprises an antibody that binds a Natural Killer (NK) cell antigen.
150. The method of embodiment 149, wherein the NK cell antigen is CD7, CD8, or CD56.
151. The method of any one of embodiments 138 to 150, wherein the antibody is a human or humanized antibody.
152. The method of any one of embodiments 138 to 151, wherein the immune cell targeting group is covalently coupled to a lipid in the lipid blend via a polyethylene glycol (PEG) containing linker.
153. The method of embodiment 152, wherein the lipid covalently coupled to the immune cell targeting group via a PEG containing linker is distearoylglycerol (DSG), distearoyl-phosphatidylethanolamine (DSPE), dimyrstoyl-phosphatidylethanolamine (DMPE), distearoyl-glycero-phosphoglycerol (DSPG), dimyristoyl-glycerol (DMG), dipalmitoyl-phosphatidylethanolamine (DPPE), dipalmitoyl-glycerol (DPG), or ceramide.
154. The method of embodiment 152 or 153, wherein the PEG is PEG 2000.
155. The method of any one of embodiments 138 to 154, wherein the lipid-immune cell targeting group conjugate is present in the lipid blend in a range of 0.002-0.2 mole percent.
156. The method of any one of embodiments 138 to 155, wherein the ionizable cationic lipid is present in the lipid blend in a range of 40-60 mole percent.
157. The method of embodiment 138 to 156, wherein the sterol is cholesterol.
158. The method of any one of embodiments 49 to 157, wherein the sterol is present in the lipid blend in a range of 30-50 mole percent.
159. The method of claim 138 to 158, wherein the neutral phospholipid is selected from the group consisting of phosphatidylcholine, phosphatidylethanolamine, distearoyl-sn-glycero-3-phosphoethanolamine (DSPE), 1,2-distearoyl-sn-glycero-3-phosphocholine (DSPC), 1,2-dioleoyl-sn-glycero-3-phosphoethanolamine (DOPE), 1,2-dioleoyl-sn-glycero-3-phosphocholine (DOPC), sphingomyelin (SM).
160. The method of embodiment 138 to 159, wherein the neutral phospholipid is present in the lipid blend in a range of 5-15 mole percent.
161. The method of any one of embodiments 138 to 160, wherein the free PEG-lipid is selected from the group consisting of PEG-modified phosphatidylethanolamines, PEG-modified phosphatidic acids, PEG-modified ceramides, PEG-modified dialkylamines, PEG-modified diacylglycerols, and PEG-modified dialkylglycerols. For example, a PEG lipid may be PEG-dioleoylgylcerol (PEG-DOG), PEG-dimyristoyl-glycerol (PEG-DMG), PEG-dipalmitoyl-glycerol (PEG-DPG), PEG-dilinoleoyl-glycero-phosphatidyl ethanolamine (PEG-DLPE), PEG-dimyrstoyl-phosphatidylethanolamine (PEG-DMPE), PEG-dipalmitoyl-phosphatidylethanolamine (PEG-DPPE), PEG-distearoylglycerol (PEG-DSG), PEG-diacylglycerol (PEG-DAG, e.g., PEG-DMG, PEG-DPG, and PEG-DSG), PEG-ceramide, PEG-distearoyl-glycero-phosphoglycerol (PEG-DSPG), PEG-dioleoyl-glycero-phosphoethanolamine (PEG-DOPE), 2-[(polyethylene glycol)-2000]-N,N-ditetradecylacetamide, or a PEG-distearoyl-phosphatidylethanolamine (PEG-DSPE) lipid.
162. The method of embodiments 138 to 160, wherein the free PEG-lipid comprises a diacylphosphatidylethanolamines comprising dimyristoyl (C14) chain, Dipalmitoyl (C16) chain or Distearoyl (C18) chain.
163. The method of any one of embodiments 138 to 162, wherein the free PEG-lipid is present in the lipid blend in a range of 0.5-2.5 mole percent.
164. The method of any one of embodiments 138 to 163, wherein the free PEG-lipid comprises the same or a different lipid as the lipid in the lipid-immune cell targeting group conjugate.
165. The method of embodiments 138 to 164, wherein the LNP has a mean diameter in the range of 50-200 nm.
166. The method of embodiment 165, where the LNP has a mean diameter of about 100 nm.
167. The method of embodiments 138 to 166, wherein the LNP has a polydispersity index in a range from 0.05 to 1.
168. The method of embodiments 138 to 167, wherein the LNP has a zeta potential of from about +10 mV to about +30 mV at pH 5.
169. The method of embodiments 138 to 168, wherein the nucleic acid is DNA or RNA.
170. The method of embodiment 169, wherein the RNA is an mRNA, tRNA, siRNA, gNRA, or microRNA.
171. The method of embodiment 170, wherein the mRNA encodes a receptor, a growth factor, a hormone, a cytokine, an antibody, an antigen, an enzyme, or a vaccine.
172. The method of embodiment 170, wherein the mRNA encodes a polypeptide capable of regulating immune response in the immune cell.
173. The method of embodiment 170, wherein the mRNA encodes a polypeptide capable of reprogramming the immune cell.
174. The method of embodiment 170, wherein the mRNA encodes a synthetic T cell receptor (synTCR) or a Chimeric Antigen Receptor (CAR).
175. The method of any one of embodiments 138 to 174, wherein the immune cell targeting group comprises an antibody, and the antibody is a Fab or an immunoglobulin single variable domain.
176. The method of any one of embodiments 138 to 174, wherein the immune cell targeting group comprises an antibody fragment selected from the group consisting of a Fab, F(ab′)2, Fab′-SH, Fv, and scFv fragment.
177. The method of embodiment 175 or 176, wherein the immune cell targeting group comprises a Fab that comprises one or more interchain disulfide bonds.
178. The method of embodiment 177, wherein the Fab comprises a heavy chain fragment that comprises F174C and C233S substitutions, and a light chain fragment that comprises S176C and C214S substitutions, numbering according to Kabat.
179. The method of any one of embodiments 175 to 178, wherein the immune cell targeting group comprises a Fab that comprises a cysteine at the C-terminus of the heavy or light chain fragment.
180. The method of embodiment 175, wherein the Fab further comprises one or more amino acids between the heavy chain fragment of the Fab and the C-terminal cysteine.
181. The method of any one of embodiments 176 to 180, wherein the Fab comprises a heavy chain variable domain linked to an antibody CH1 domain and a light chain variable domain linked to an antibody light chain constant domain, wherein the CH1 domain and the light chain constant domain are linked by one or more interchain disulfide bonds, and wherein the immune cell targeting group further comprises a single chain variable fragment (scFv) linked to the C-terminus of the light chain constant domain by an amino acid linker.
182. The method of embodiment 175, wherein the immune cell targeting group comprises an immunoglobulin single variable domain.
183. The method of embodiment 175 or 182, wherein the immunoglobulin single variable domain comprises a cysteine at the C-terminus.
184. The method of embodiment 183, wherein the immunoglobulin single variable domain comprises a V_{HH} domain and further comprises a spacer comprising one or more amino acids between the V_{HH} domain and the C-terminal cysteine.
185. The method of any one of embodiments 175 and 182 to 184, wherein the immune cell targeting group comprises two or more V_{HH} domains.
186. The method of embodiment 185, wherein the two or more V_{HH} domains are linked by an amino acid linker.
187. The method of embodiment 185, wherein the immune cell targeting group comprises a first V_{HH} domain linked to an antibody CH1 domain and a second V_{HH} domain linked to an antibody light chain constant domain, and wherein the antibody CH1 domain and the antibody light chain constant domain are linked by one or more disulfide bonds.
188. The method of any one of embodiments 175, and 182 to 184, wherein the immune cell targeting group comprises a V_{HH} domain linked to an antibody CH1 domain, and wherein the antibody CH1 domain is linked to an antibody light chain constant domain by one or more disulfide bonds.
189. The method of embodiment 185 or 186, wherein the CH1 domain comprises F174C and C₂₃₃S substitutions, and the light chain constant domain comprises S176C and C₂₁₄S substitutions, numbering according to Kabat.
190. The method of any one of embodiments 138 to 174, wherein the immune cell targeting group comprises a Fab that comprises:
(a) a heavy chain fragment comprising the amino acid sequence of SEQ ID NO: 1 and a light chain fragment comprising the amino acid sequence of SEQ ID NO:2 or 3;
(b) a heavy chain fragment comprising the amino acid sequence of SEQ ID NO: 6 and a light chain fragment comprising the amino acid sequence of SEQ ID NO: 7.
   191. The method of any one of embodiments 138 to 190, wherein no more than 5% non-immune cells are transfected by the LNP.
   192. The method of any one of embodiments 138 to 191, wherein half-life of the nucleic acid delivered by the LNP or a polypeptide encoded by the nucleic acid delivered by the LNP is at least 10% longer than half-life of nucleic acid delivered by a reference LNP or a polypeptide encoded by the nucleic acid delivered by the reference LNP.
   193. The method of any one of embodiments 138 to 192, wherein at least 10% immune cells are transfected by the LNP.
   194. The method of any one of embodiments 138 to 193, wherein expression level of the nucleic acid delivered by the LNP is at least 10% higher than expression level of nucleic acid delivered by a reference LNP.

### EXAMPLES

The invention now being generally described, will be more readily understood by reference to the following examples, which are included merely for purposes of illustration of certain aspects and embodiments of the present invention, and are not intended to limit the invention.

### Example 1. Preparation of Ionizable Cationic Lipids

This Example describes the synthesis of various cationic lipids.

### General Scheme for the Synthesis of Lipids 1 Through Lipid 30

A general scheme for the synthesis of Lipids 1 through Lipid 30 is provided in Scheme 1 below. Corresponding R and R′ for each lipid is provided in Tables 2 to 4 below.

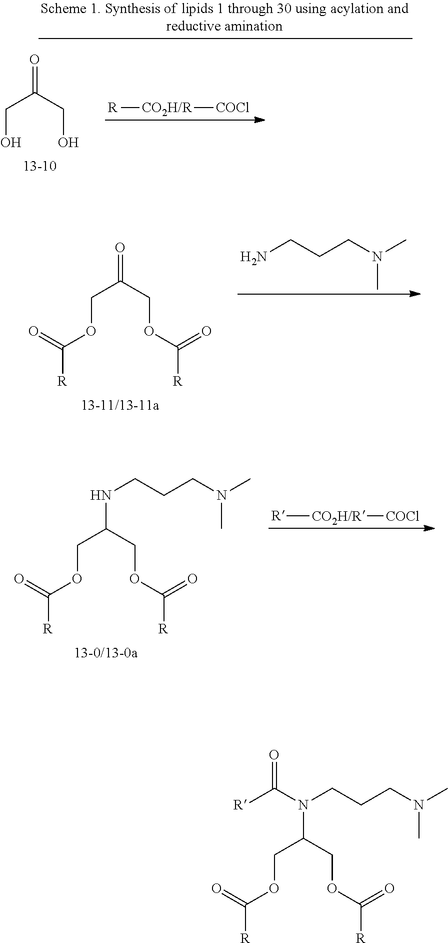

### Synthesis of Intermediates 13-11 and 13-11a

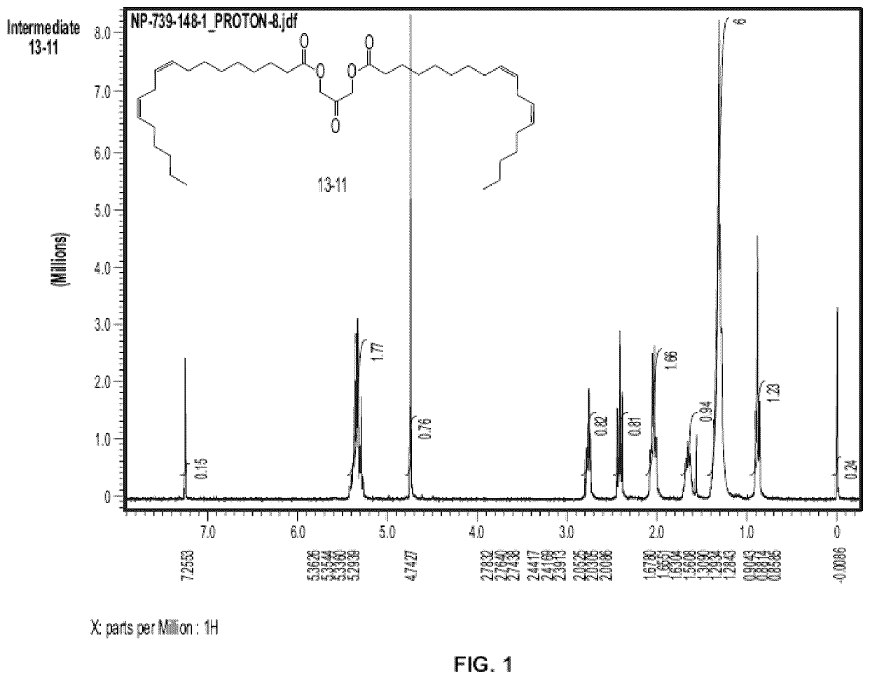

Intermediate 13-11 (Scheme 2) was synthesized by acylation of dihydroxyacetone (13-10) with linoleic acid. Dihydroxyacetone (22 mmol, 2 g, 1 eq.) was reacted with linoleic acid 1-5 (55 mmol, 15.4 g, 2.5 eq.) using EDCI (55 mmol, 10.5 g, 2.5 eq.) activation in 50 mL DCM, in the presence of DIPEA (55 mmol, 9.6 mL, 2.5 eq.), DMAP (4.4 mmol, 540 mg, 0.2 eq.) at room temperature yielding 11.1 g (79%) crude product. Purified product was obtained by column chromatography and characterized by proton NMR spectroscopy (FIG. 1).

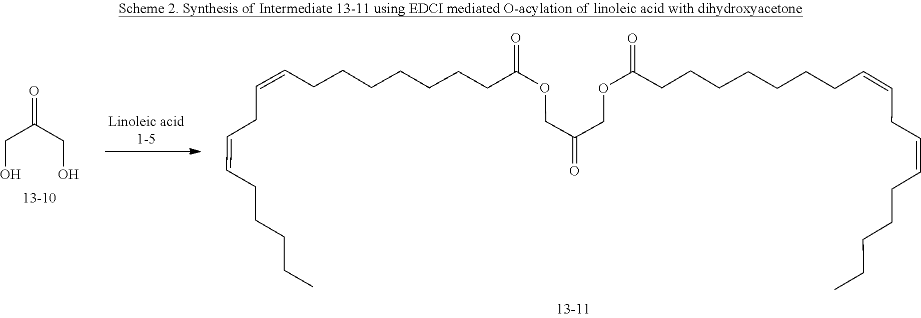 [SMILES: O=C(CO)CO ; CCCCC/C=C\C/C=C\CCCCCCCC(=O)OCC(=O)COC(=O)CCCCCCC/C=C\C/C=C\CCCCC]

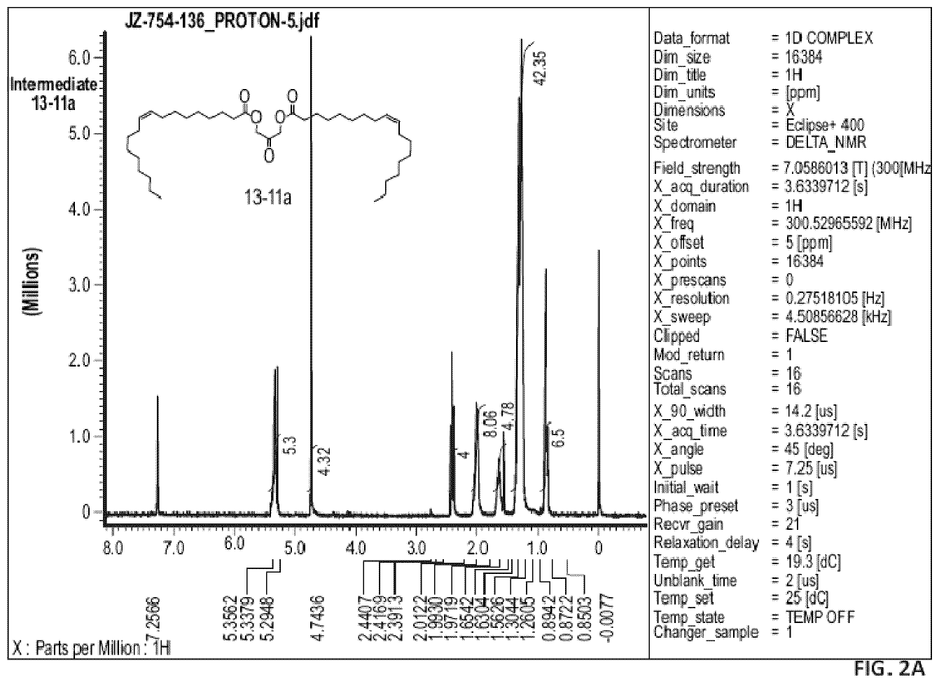

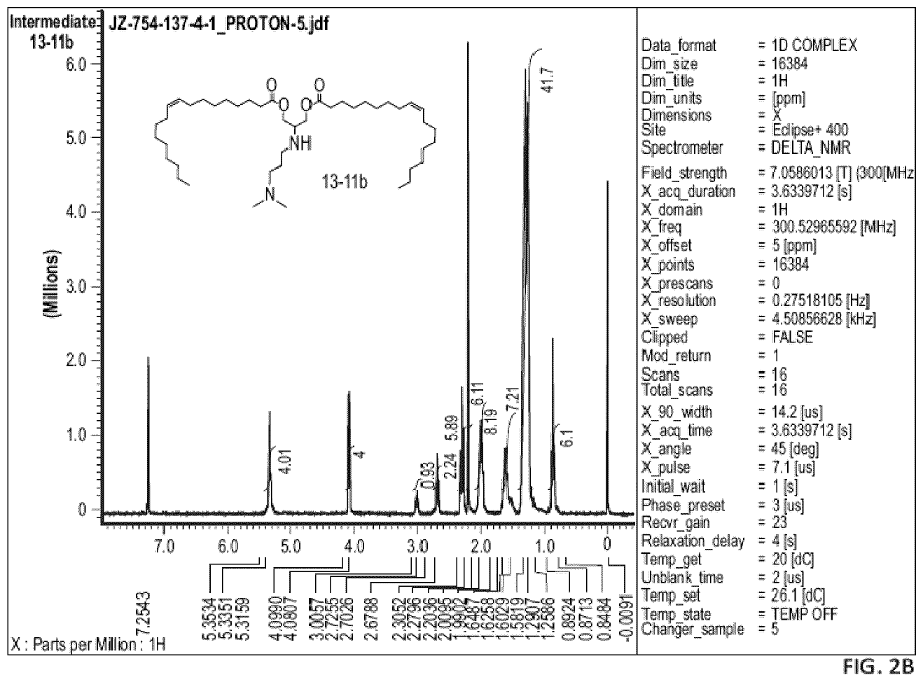

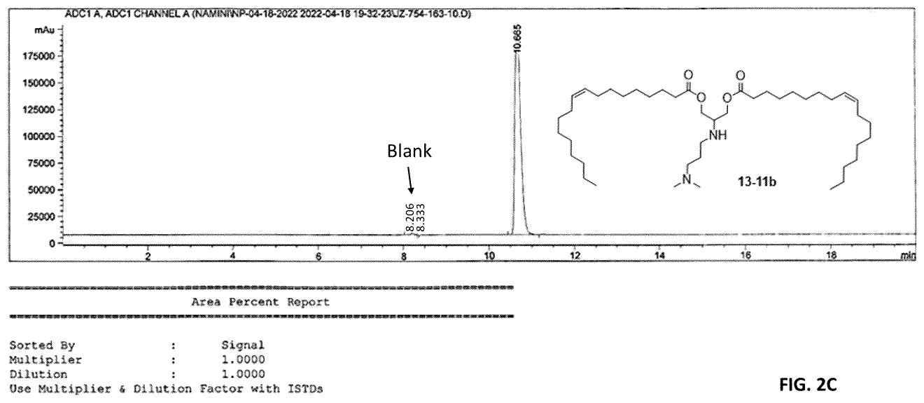

Intermediates 13-11a (Scheme 3) was synthesized by acylation of dihydroxyacetone (13-10) with oleoyl chloride. Dihydroxyacetone (44.4 mmol, 4 g, 1 eq.) was reacted with oleoyl chloride 1-6a (111 mmol, 36.7 mL, 2.5 eq.) in the presence of Pyridine (133.3 mmol, 11 mL, 3 eq.), DMAP (13.3 mmol, 1.63 g, 0.3 eq.) in 80 mL DCM, at room temperature yielding 14.9 g (54%) crude product. Crude product was purified by column chromatography and characterized by proton NMR spectroscopy (FIG. 2A).

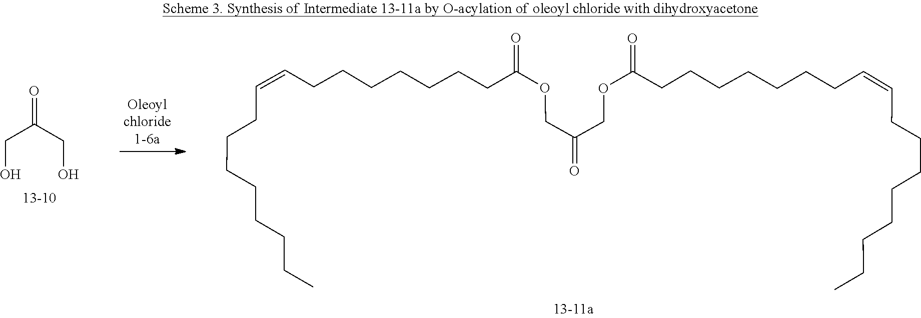 [SMILES: O=C(CO)CO ; CCCCCCCC/C=C\CCCCCCCC(=O)OCC(=O)COC(=O)CCCCCCC/C=C\CCCCCCCC]

### Synthesis of intermediates 13-0a and 13-11b

Intermediates 13-0a and 13-11b were synthesized by reductive amination of intermediates 13-11 and 13-11a respectively.

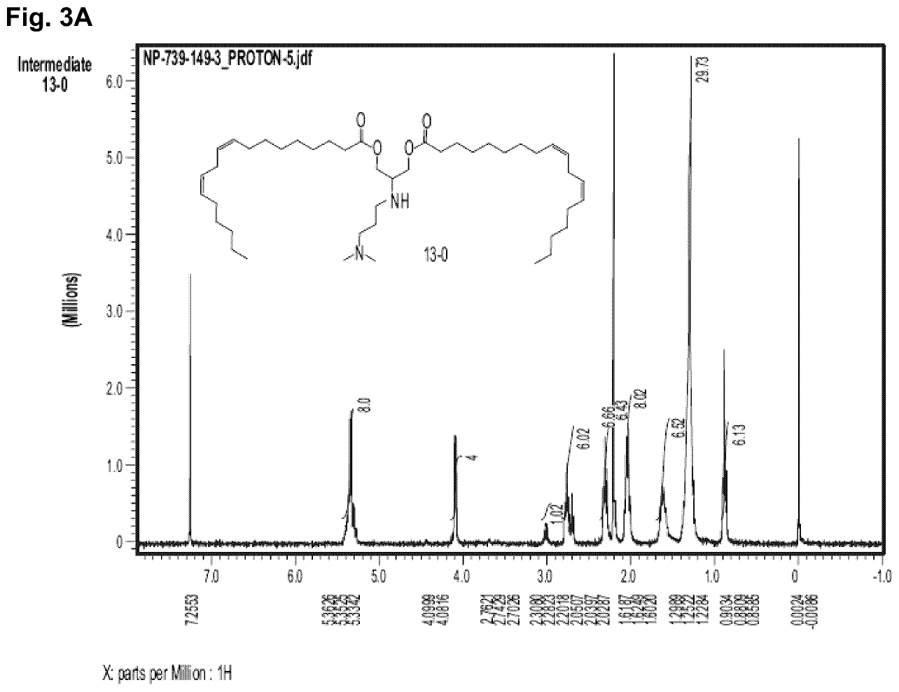

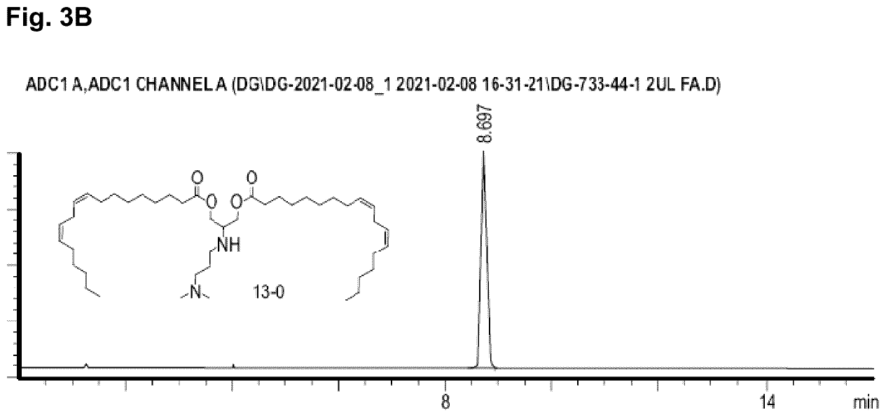

Intermediate 13-0 was produced by reductive amination (scheme 4) of intermediate 13-11 (13.1 mmol, 8.1 g, 1.0 eq) using N1,N1-dimethylpropane-1,3-diamine 15-3 (26 mmol, 3.2 mL, 2.0 eq.) in DCM (10 mL) using acetic acid (26.0 mmol, 1.50 mL, 2 eq.) and sodium borohydride triacetate (4.32 mmol, 3.3 g, 1.2 eq.) yielding 3.1 g (32%) crude product. Column purification resulted in pure product (Proton NMR Spectrum and LC-CAD chromatogram shown in FIG. 3A and FIG. 3B, respectively).

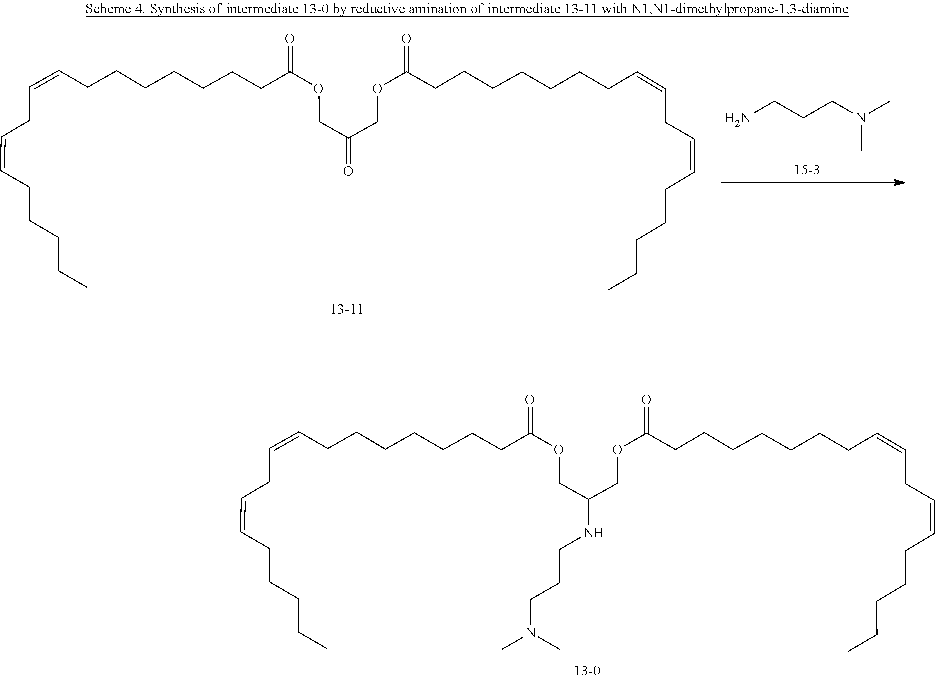

Intermediate 13-11b was produced by reductive amination (scheme 5) of intermediate 13-11a (24.2 mmol, 14.9 g, 1.0 eq) using N1,N1-dimethylpropane-1,3-diamine 15-3 (48.4 mmol, 6.05 mL, 2.0 eq.) in DCM (60 mL) using acetic acid (48.4 mmol, 2.8 mL, 2 eq.) and sodium borohydride triacetate (29.1 mmol, 6.05 g, 1.2 eq.) yielding 6 g (35%) crude product. Column purification resulted in purified product (Proton NMR Spectrum and LC-ELSD chromatogram shown in FIG. 2B and FIG. 2C, respectively).

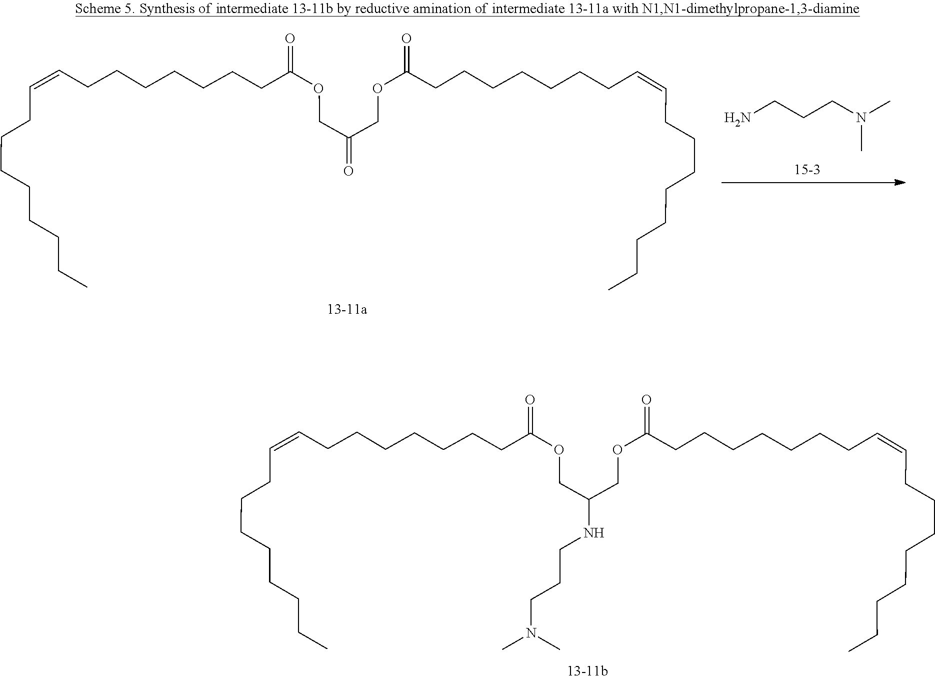

TABLE 2
R (O-acyl) and R′ (N-acyl) groups of lipids 1 through 8

| Lipid # | R (O-acyl groups) | R′ (N-acyl group) |
|---|---|---|
| Lipid 1 | 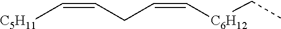 | 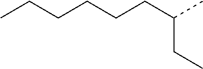 |
| Lipid 2 | 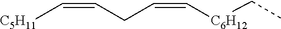 | 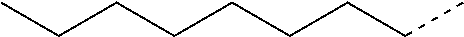 |
| Lipid 3 | 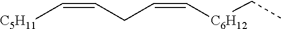 | 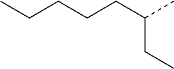 |
| Lipid 4 | 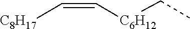 | 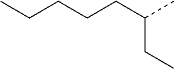 |
| Lipid 5 | 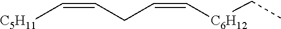 | 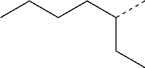 |
| Lipid 6 | 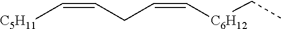 | 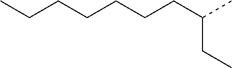 |
| Lipid 7 | 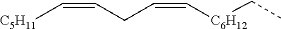 | 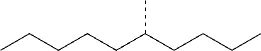 |
| Lipid 8 | 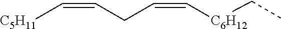 | 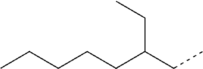 |

TABLE 3
R (O-acyl) and R′ (N-acyl) groups of Lipids 9 through 16

| Lipid # | R (O-acyl groups) | R′ (N-acyl group) |
|---|---|---|
| Lipid 9 | 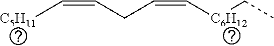 | 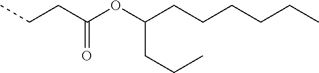 |
| Lipid 10 | 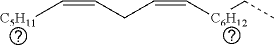 | 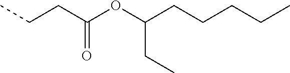 |
| Lipid 10A | 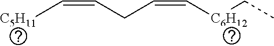 | 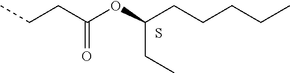 |
| Lipid 11 | 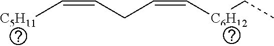 | 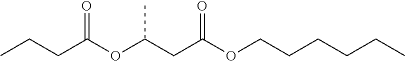 [SMILES: CCCCCCOC(=O)C[C@@H](C)OC(=O)CCC] |
| Lipid 11A | 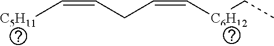 | 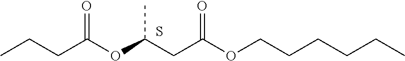 |
| Lipid 12 | 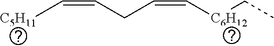 | 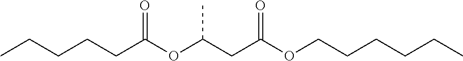 [SMILES: CCCCCCOC(=O)C[C@@H](C)OC(=O)CCCCC] |
| Lipid 13 | 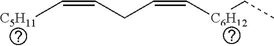 | 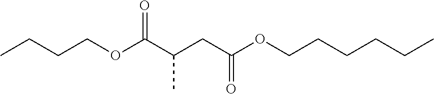 [SMILES: CCCCCCOC(=O)C[C@H](C)C(=O)OCCCC] |
| Lipid 14 | 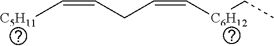 | 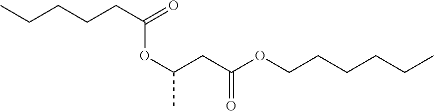 [SMILES: CCCCCCOC(=O)C[C@H](C)OC(=O)CCCCC] |
| Lipid 14A | 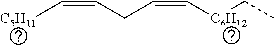 | 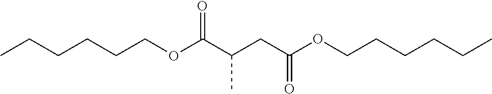 [SMILES: CCCCCCOC(=O)C[C@H](C)C(=O)OCCCCCC] |
| Lipid 15 | 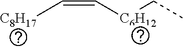 | 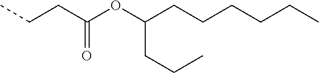 |
| Lipid 16 | 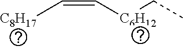 | 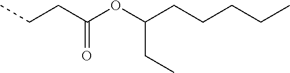 |

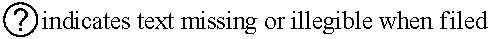

TABLE 4-1
R (O-acyl) and R′ (N-acyl) groups of Lipids 17, 17A, 18, 18A, 19, 19A, 20, 20A, 21, 21A, 22, 23, and 23A

| Lipid # | R (O-acyl groups) | R′ (N-acyl group) |
|---|---|---|
| Lipid 17 | 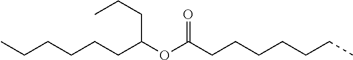 | 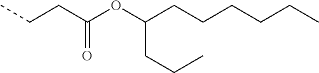 |
| Lipid 17A | 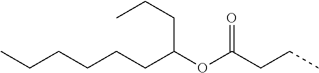 | 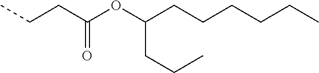 |
| Lipid 18 | 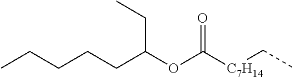 | 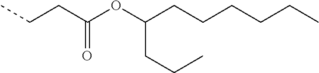 |
| Lipid 18A | 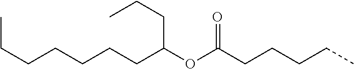 | 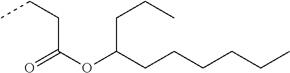 |
| Lipid 19 | 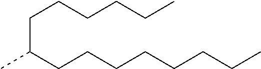 | 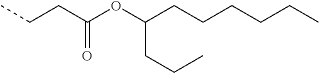 |
| Lipid 19A | 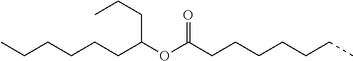 | 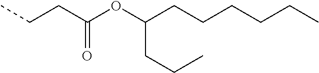 |
| Lipid 20 | 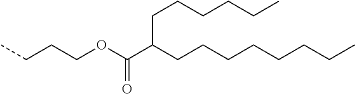 | 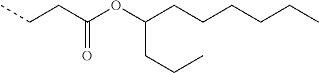 |
| Lipid 20A | 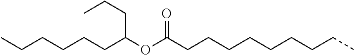 | 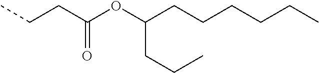 |
| Lipid 21 | 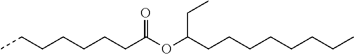 | 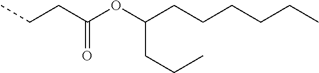 |
| Lipid 21A | 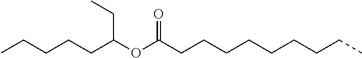 | 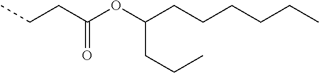 |
| Lipid 22 | 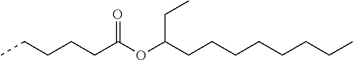 | 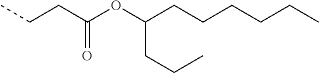 |
| Lipid 23 | 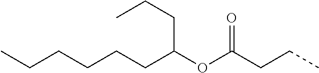 | 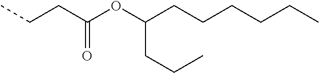 |
| Lipid 23A | 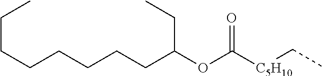 | 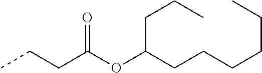 |

TABLE 4-2
R (O-acyl) and R′ (N-acyl) groups of Lipids 24, 25, 25A, 26, 27, 28, 29, and 30

| Lipid # | R (O-acyl groups) | R′ (N-acyl group) |
|---|---|---|
| Lipid 24 | 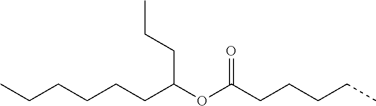 | 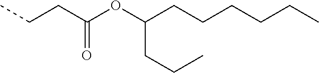 |
| Lipid 25 | 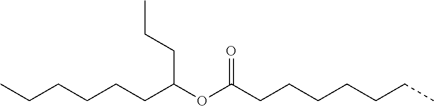 | 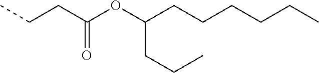 |
| Lipid 25A | 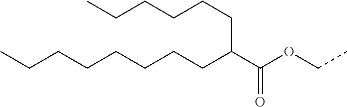 | 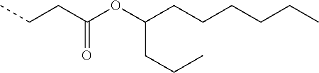 |
| Lipid 26 | 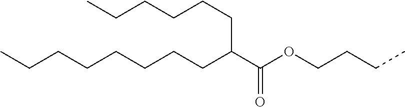 | 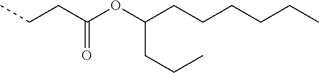 |
| Lipid 27 | 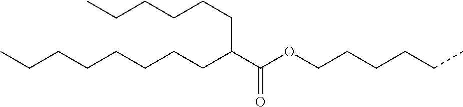 | 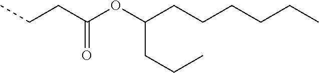 |
| Lipid 28 | 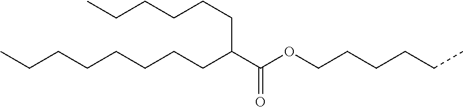 | 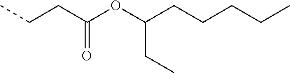 |
| Lipid 29 | 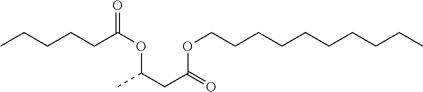 [SMILES: CCCCCCCCCCOC(=O)C[C@H](C)OC(=O)CCCCC] | 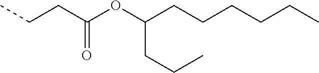 |
| Lipid 30 | 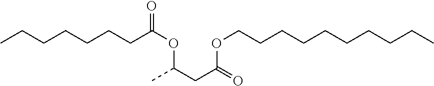 [SMILES: CCCCCCCCCCOC(=O)C[C@H](C)OC(=O)CCCCCCC] | 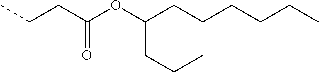 |

TABLE 4-3
R (O-acyl) and R′ (N-acyl) groups of Lipids 31 to 38, 37A, and 38A

| Lipid # | R (O-acyl groups) | R′ (N-acyl group) |
|---|---|---|
| Lipid 31 | 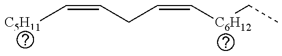 | 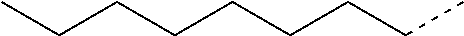 |
| Lipid 32 | 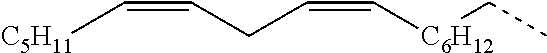 | 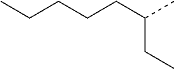 |
| Lipid 33 | 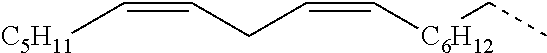 | 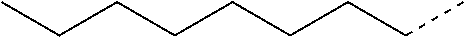 |
| Lipid 34 | 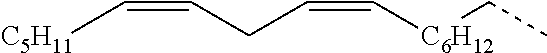 | 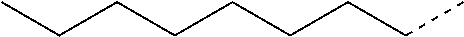 |
| Lipid 35 | 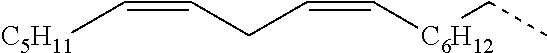 | 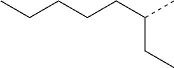 |
| Lipid 36 | 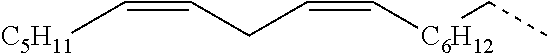 | 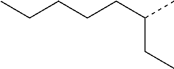 |
| Lipid 37 | 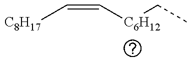 | 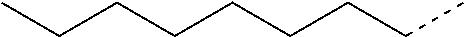 |
| Lipid 38 | 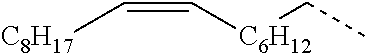 | 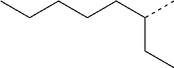 |
| Lipid 37A | 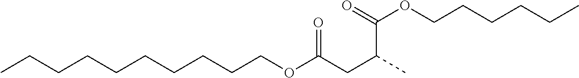 [SMILES: CCCCCCCCCCOC(=O)C[C@@H](C)C(=O)OCCCCCC] | 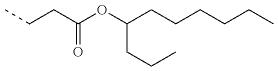 |
| Lipid 38A | 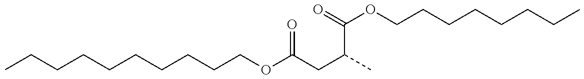 [SMILES: CCCCCCCCCCOC(=O)C[C@@H](C)C(=O)OCCCCCCCC] | 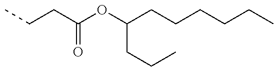 |

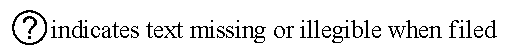

TABLE 5
Expected and observed mass (m/z) of named ionizable lipids

| Entry | Compound code | Expected mass (g/mol) | Observed mass (m/z) |
|---|---|---|---|
| 1 | Lipid 1 | 854.75 | 855.7, 856.7, 857.7 |
| | | | (M + 1, M + 2, M + 3) |
| 2 | Lipid 2 | 840.73 | 841.7, 842.7, 843.7 |
| | | | (M + 1, M + 2, M + 3) |
| 3 | Lipid 3 | 840.73 | 841.7, 842.7, 843.7 |
| | | | (M + 1, M + 2, M + 3) |
| 4 | Lipid 4 | 845.39 | 845.7, 846.7, 847.7 |
| | | | (M, M + 1, M + 2) |
| 5 | Lipid 5 (S) isomer | 827.33 | 827.7, 828.7, 829.7 |
| | (Lipid 5A) | | (M, M + 1, M + 2) |
| 6 | Lipid 6 | 868.76 | 869.7, 870.7, 871.7 |
| | | | (M + 1, M + 2, M + 3) |
| 7 | Lipid 7 | 868.76 | 869.7, 870.7, 871.7 |
| | | | (M + 1, M + 2, M + 3) |
| 8 | Lipid 8 | 854.75 | 855.7, 856.7, 857.7 |
| | | | (M + 1, M + 2, M + 3) |
| 9 | Lipid 9 | 940.78 | 941.7, 942.7, 943.7 |
| | | | (M + 1, M + 2, M + 3) |
| 10 | Lipid 10 (S) isomer | 912.75 | 913.7, 914.7, 915.7 |
| | (Lipid 10A) | | (M + 1, M + 2, M + 3) |
| 11 | Lipid 11 (S) isomer | 970.76 | 971.7, 972.7, 973.7 |
| | (Lipid 11A) | | (M + 1, M + 2, M + 3) |
| 12 | Lipid 12 | 999.51 | 999.0, 1001, 1002 |
| | | | (M + 1, M + 2, M + 3) |
| 13 | Lipid 13 | 984.77 | 985.7, 986.7, 987.6 |
| | | | (M + 1, M + 2, M + 3) |
| 14 | Lipid 14A | 1013.54 | 1013.1, 1014.1, |
| | | | 1015.1 |
| | | | (M + 1, M + 2, M + 3) |
| 15 | Lipid 15 | 944.82 | 945.1, 946.1, 947.1 |
| | | | (M + 1, M + 2, M + 3) |
| 16 | Lipid 16 | 916.78 | 917.2, 918.2, 919.2 |
| | | | (M + 1, M + 2, M + 3) |
| 17 | Lipid 17A | 896.67 | 897.9, 898.9, 899.9 |
| | | | (M + 1, M + 2, M + 3) |
| 18 | Lipid 18A | 952.73 | 953.7, 954.7, 955.7 |
| | | | (M + 1, M + 2, M + 3) |
| 19 | Lipid 19A | 1008.80 | 1009.8, 1010.8, |
| | | | 1011.8 |
| | | | (M + 1, M + 2, M + 3) |
| 20 | Lipid 20A | 1064.9 | 1065.7, 1066.7, |
| | | | 1067.7 |
| | | | (M + 1, M + 2, M + 3) |
| 21 | Lipid 21A | 1008.80 | 1009.7, 1010.7, |
| | | | 1011.7 |
| | | | (M + 1, M + 2, M + 3) |
| 22 | Lipid 22 | 980.76 | 981.7, 982.7, 983.7 |
| | | | (M + 1, M + 2, M + 3) |
| 23 | Lipid 23A | 1036.8 | 1037.7, 1038.6, |
| | | | 1039.7 |
| | | | (M + 1, M + 2, M + 3) |
| 24 | Lipid 19 | 892.78 | 893.7, 894.7, 895.7 |
| | (Lipid 24A) | | (M + 1, M + 2, M + 3) |
| 25 | Lipid 25A | 1008.80 | 1009.7, 1010.7, |
| | | | 1011.7 |
| | | | (M + 1, M + 2, M + 3) |
| 26 | Lipid 20 | 1064.86 | 1065.1, 1066.1, |
| | (Lipid 26) | | 1067.1, 1068.1 |
| | | | (M + 1, M + 2, |
| | | | M + 3, M + 4) |
| 27 | Lipid 27 | 1120.92 | 1121.9, 1122.9, |
| | | | 1123.9 |
| | | | (M + 1, M + 2, M + 3) |
| 28 | Lipid 28 | 1092.9 | 1093.8, 1094.8, |
| | | | 1095.8 |
| | | | (M + 1, M + 2, M + 3) |
| 29 | Lipid 29 | 1124.81 | 1125.8, 1126.8, |
| | | | 1127.8 |
| | | | (M + 1, M + 2, M + 3) |
| 30 | Lipid 30 | 1180.87 | NA |
| 31 | Lipid 31 | 854.75 | 855.1, 856.1, 857.1 |
| | | | (M + 1, M + 2, M + 3) |
| 32 | Lipid 32 | 854.75 | 855.7, 856.7, 857.7 |
| | | | (M + 1, M + 2, M + 3) |
| 33 | Lipid 33 | 840.73 | 841.7, 842.7, 843.7 |
| | | | (M + 1, M + 2, M + 3) |
| 34 | Lipid 34 | 868.76 | 869.7, 870.7, 871.7 |
| | | | (M + 1, M + 2, M + 3) |
| 35 | Lipid 35 | 914.77 | NA |
| 36 | Lipid 36 | 884.76 | NA |
| 37 | Lipid 37A | 1153.63 | 1153.8, 1154.8, |
| | | | 1155.8 |
| | | | (M + 1, M + 2, M + 3) |
| 38 | Lipid 38A | 1209.74 | NA |

### Synthesis of Lipids 1-16 by N-Acylation of Intermediates 13-0 or 13-11b

N-acylation of intermediates 13-0 and 13-11b with compounds R′CO₂H or R′COCl (R′ structures shown in Table 2 and Table 3) yielded lipids 1 through 16 as described in examples below.

### Synthesis of Lipids 1, 3, 4, 5, 6, and 7 by N-Acylation of Intermediate 13-0 Using the Corresponding Acid Chlorides

### Synthesis of Lipid 1

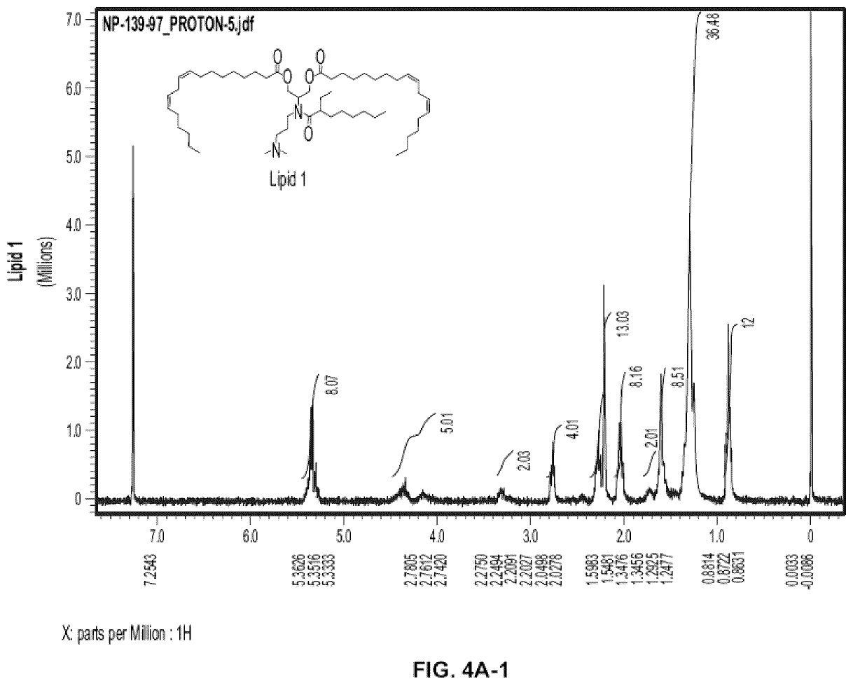

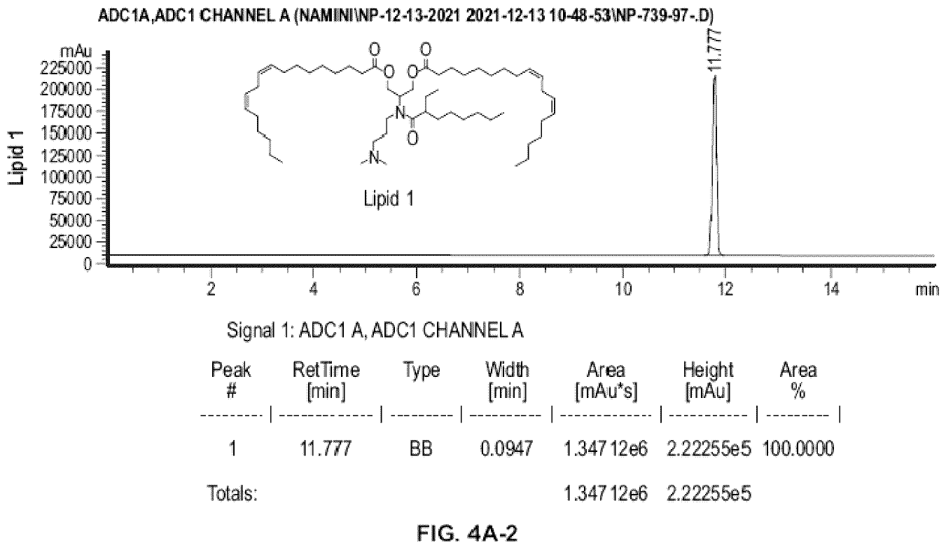

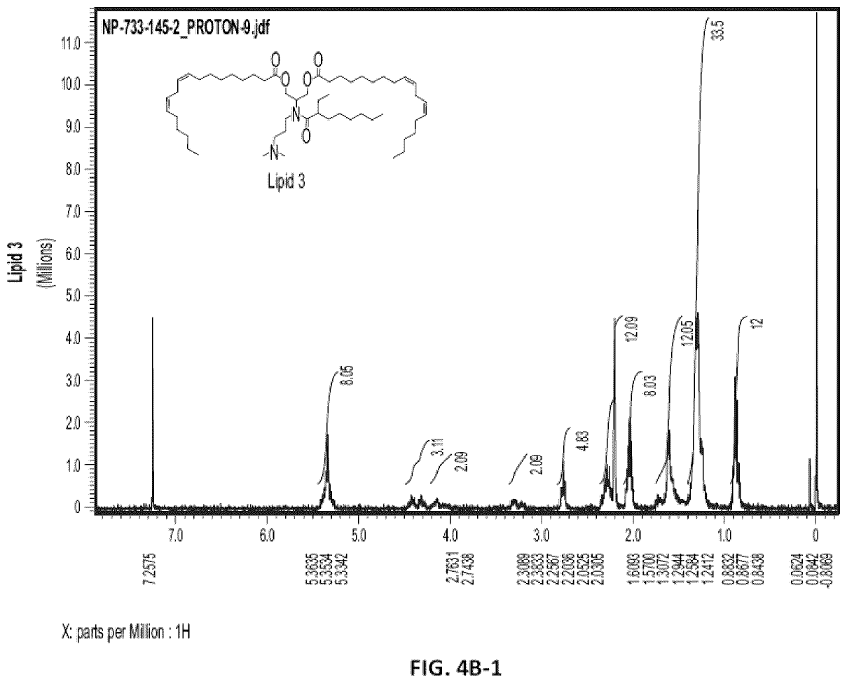

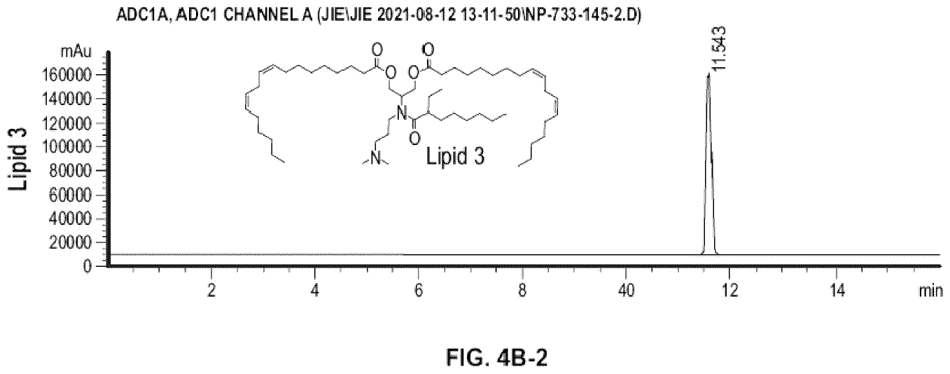

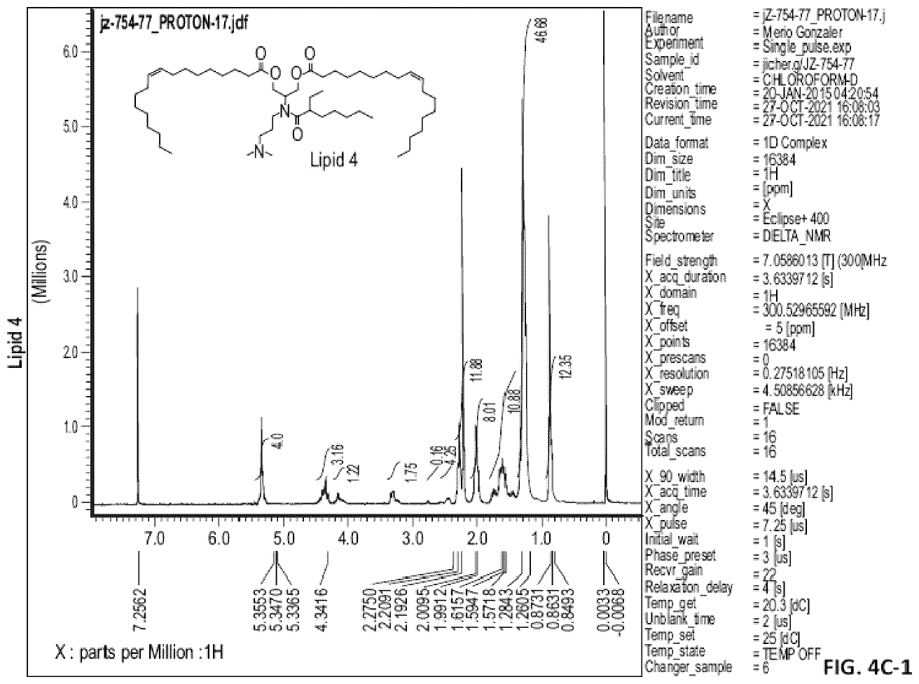

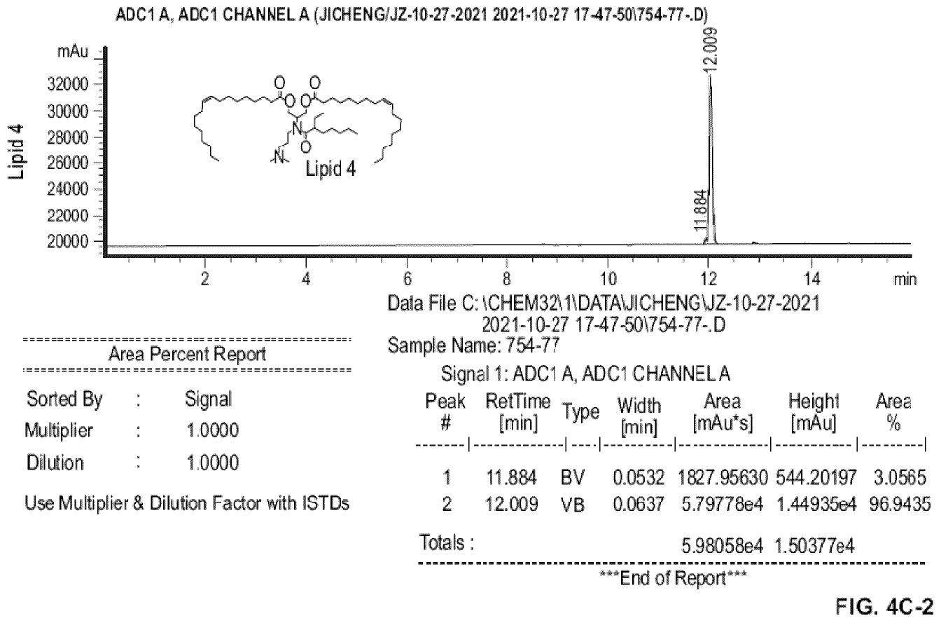

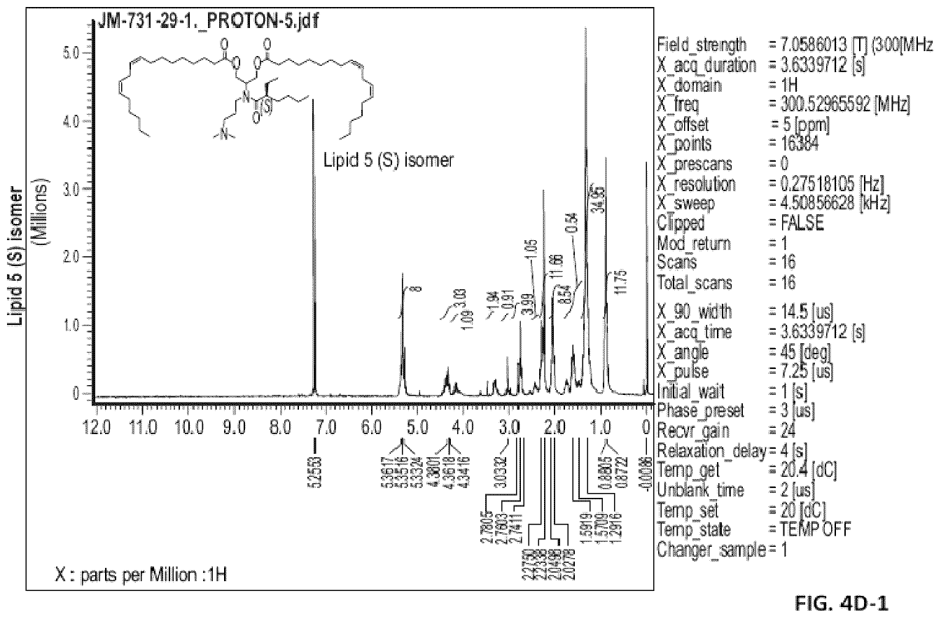

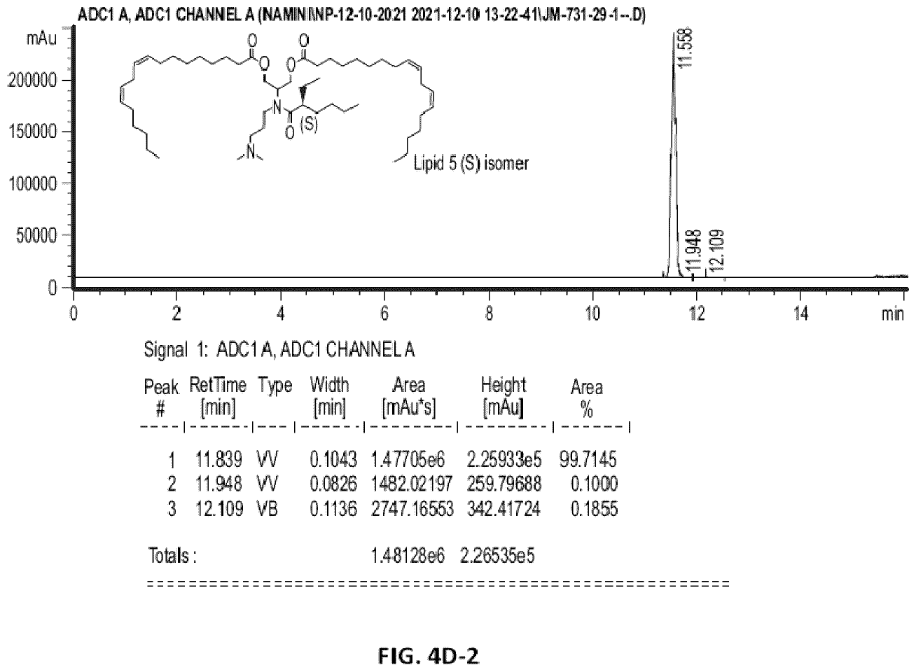

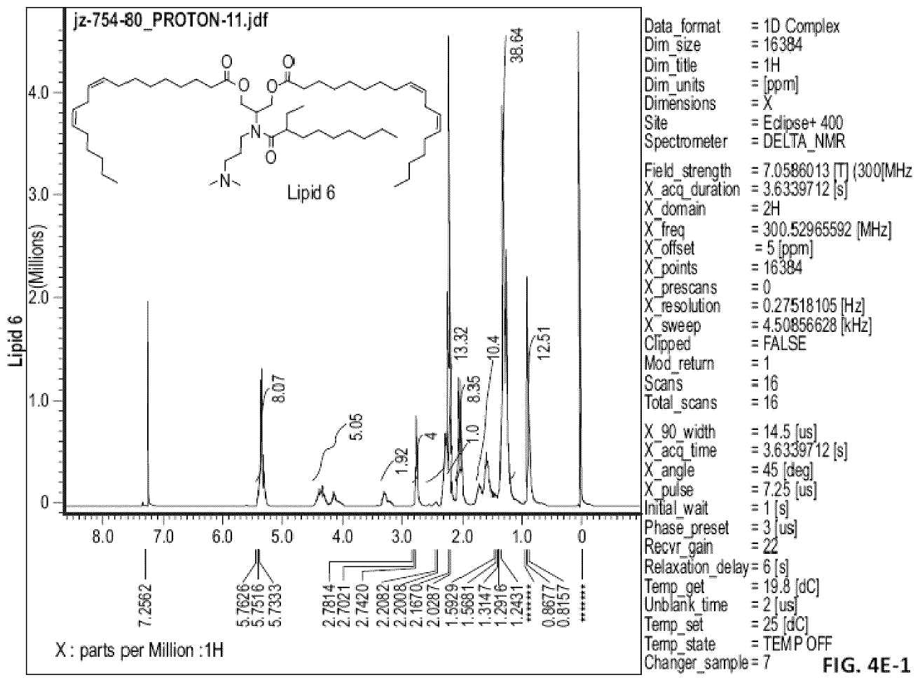

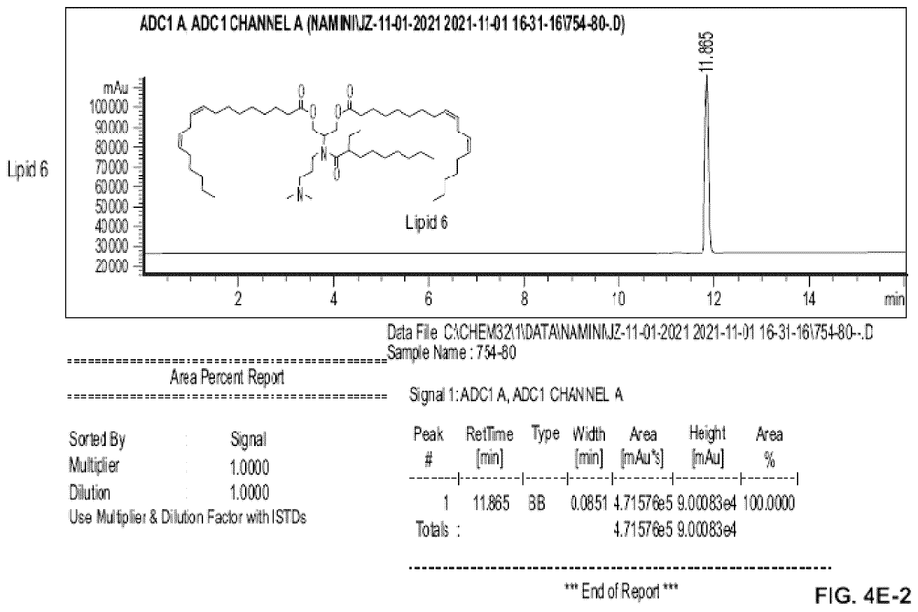

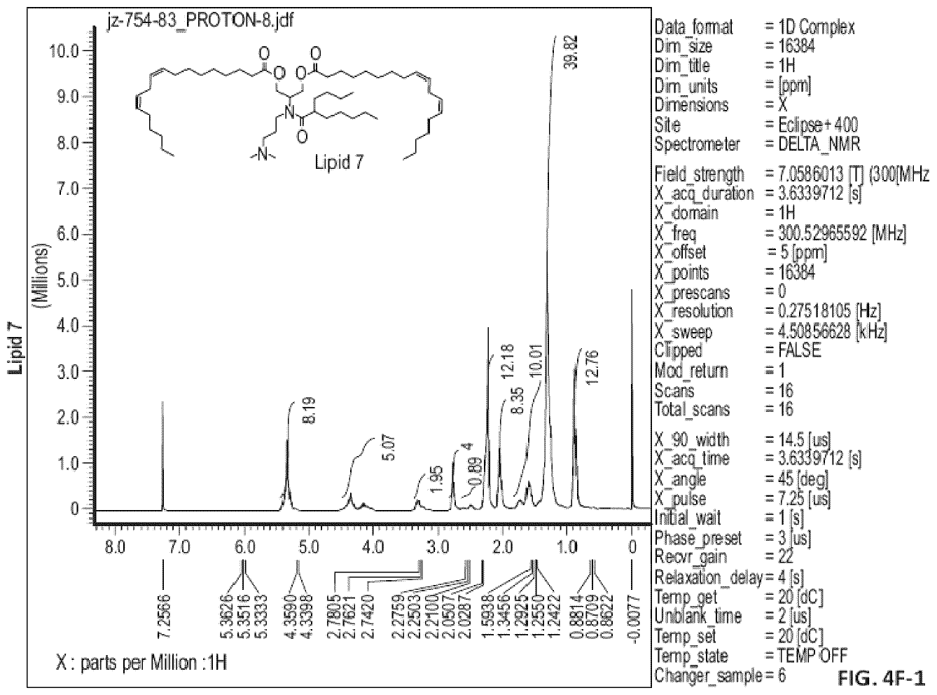

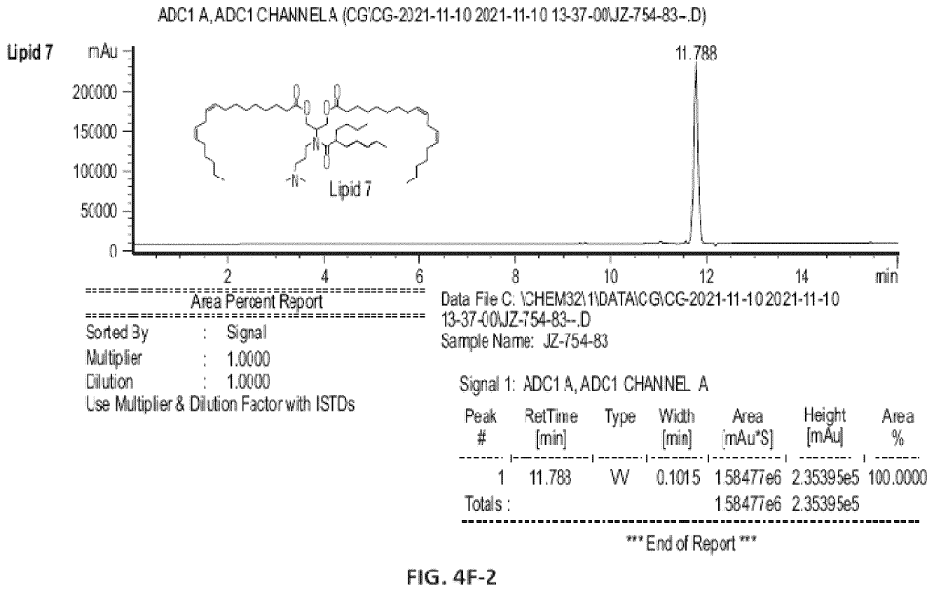

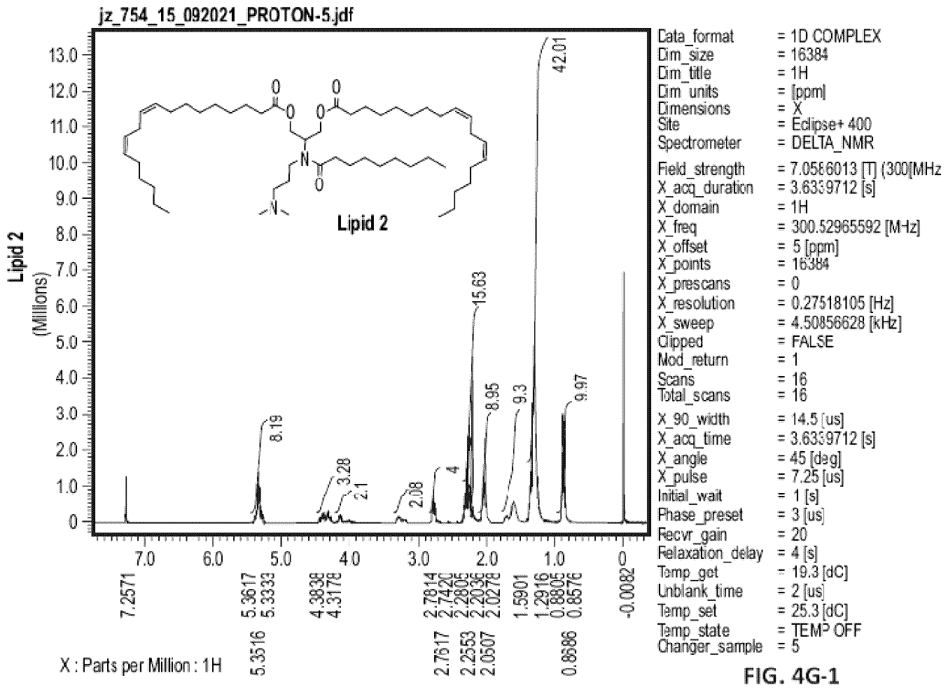

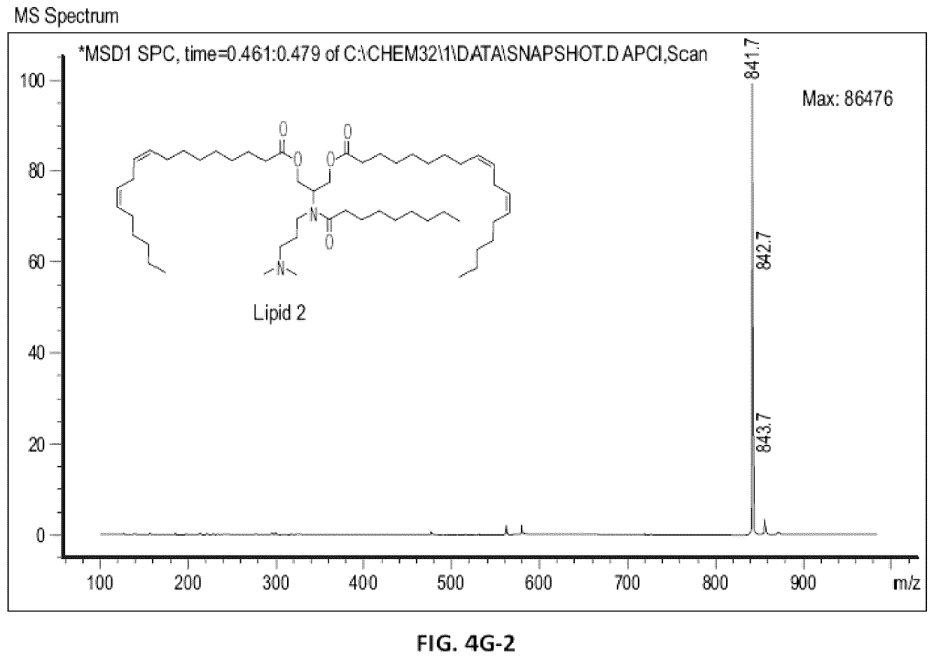

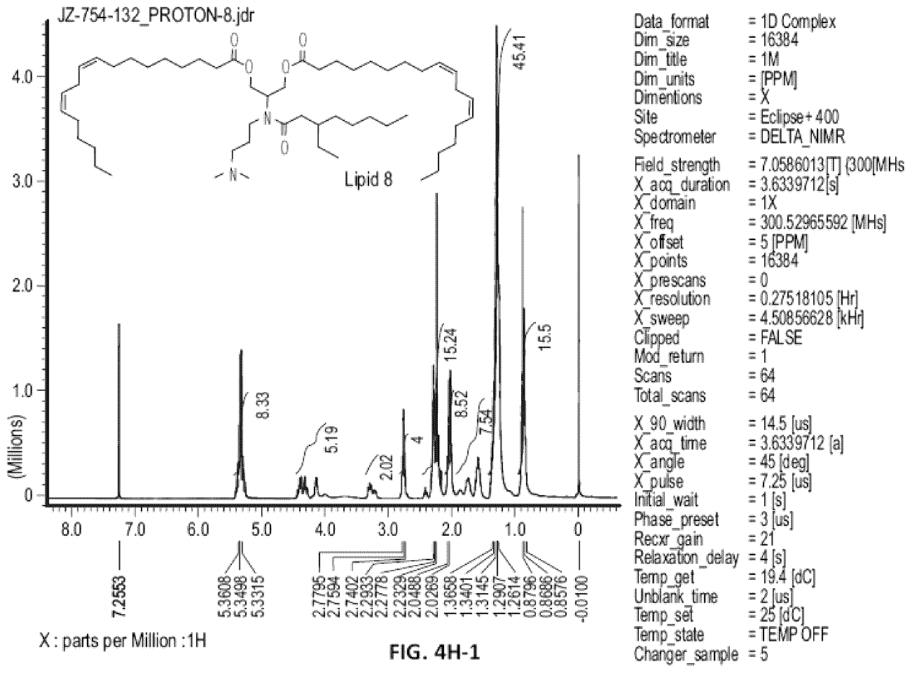

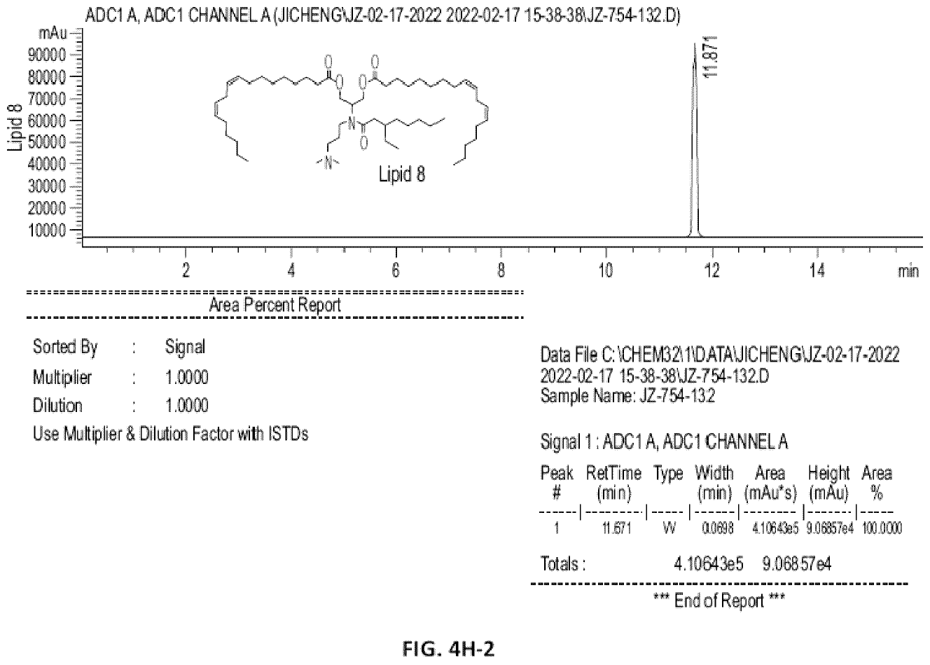

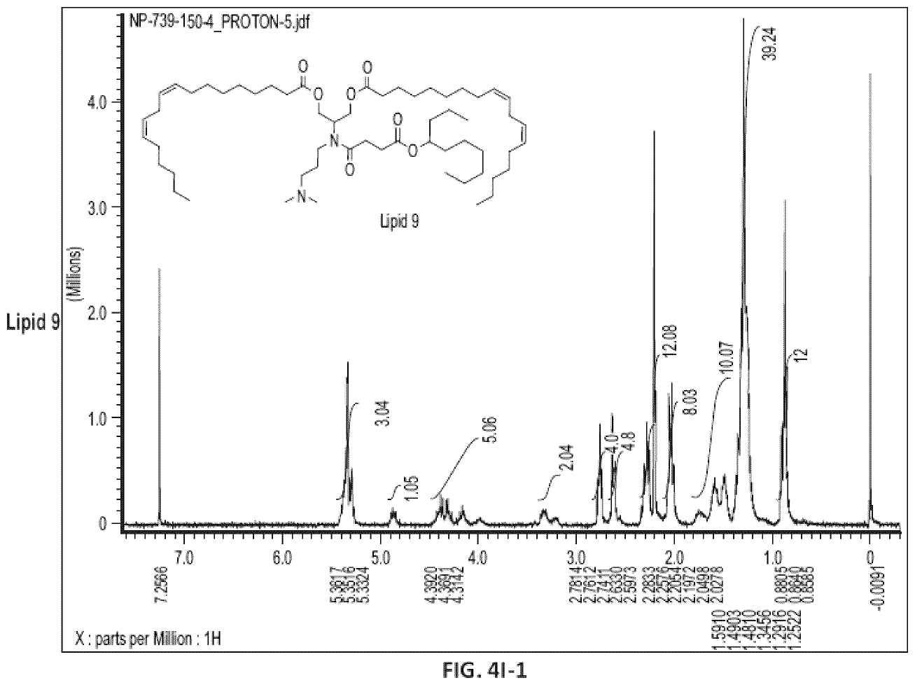

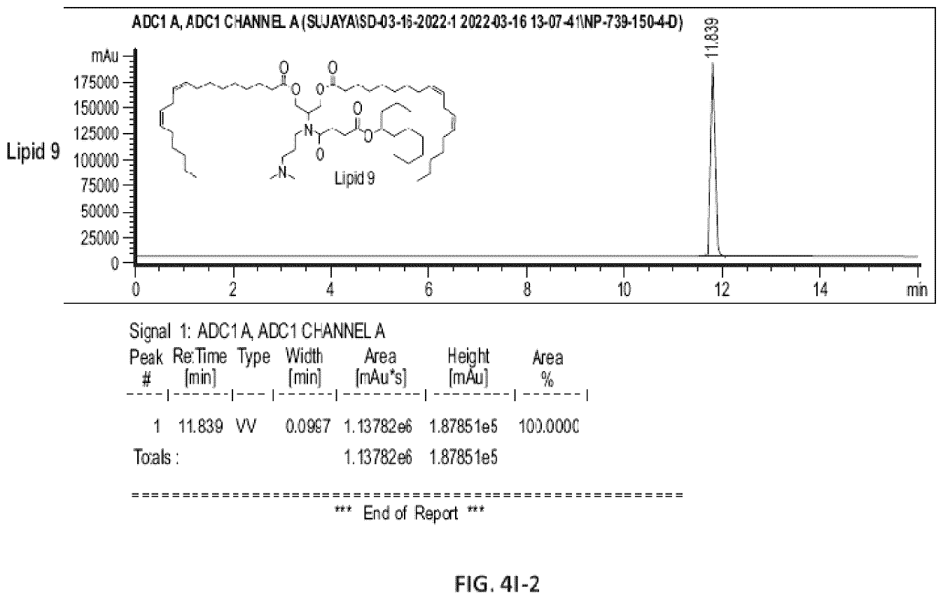

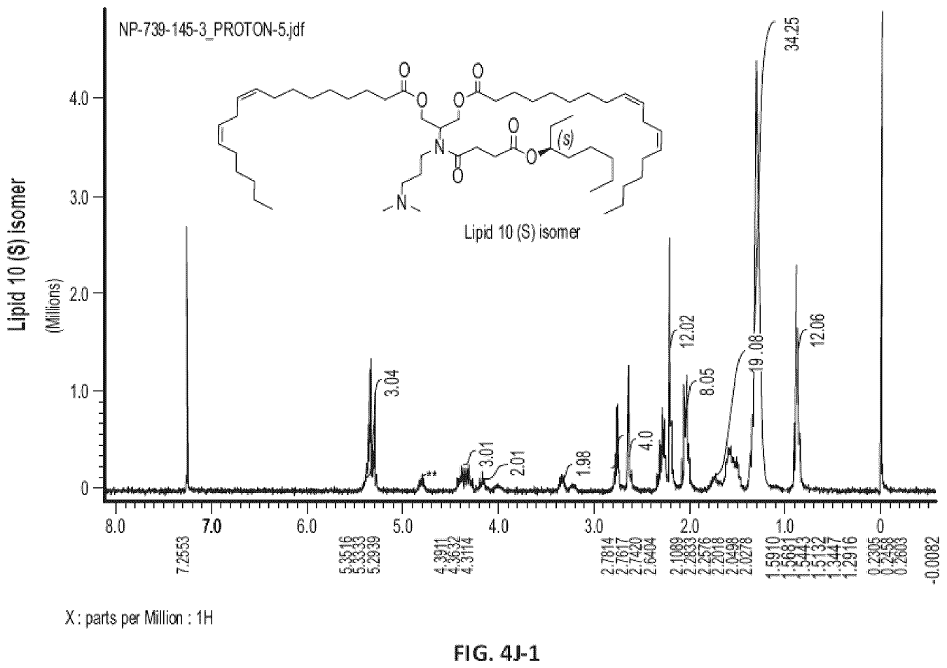

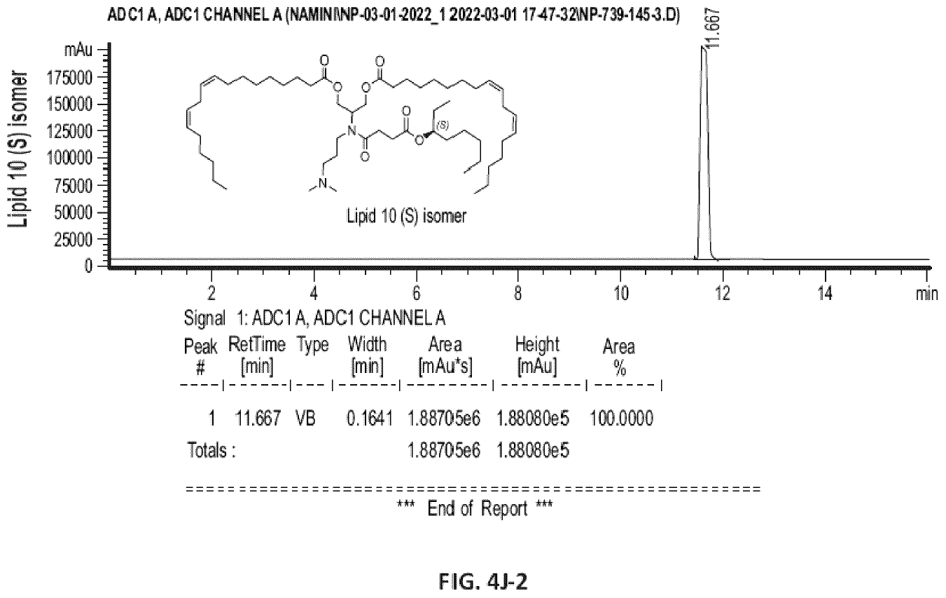

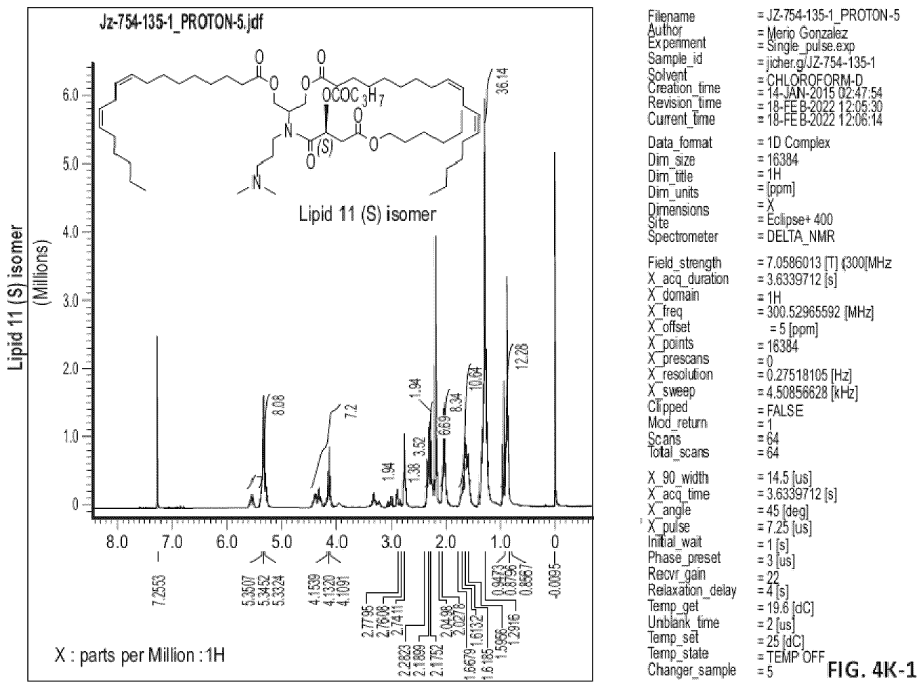

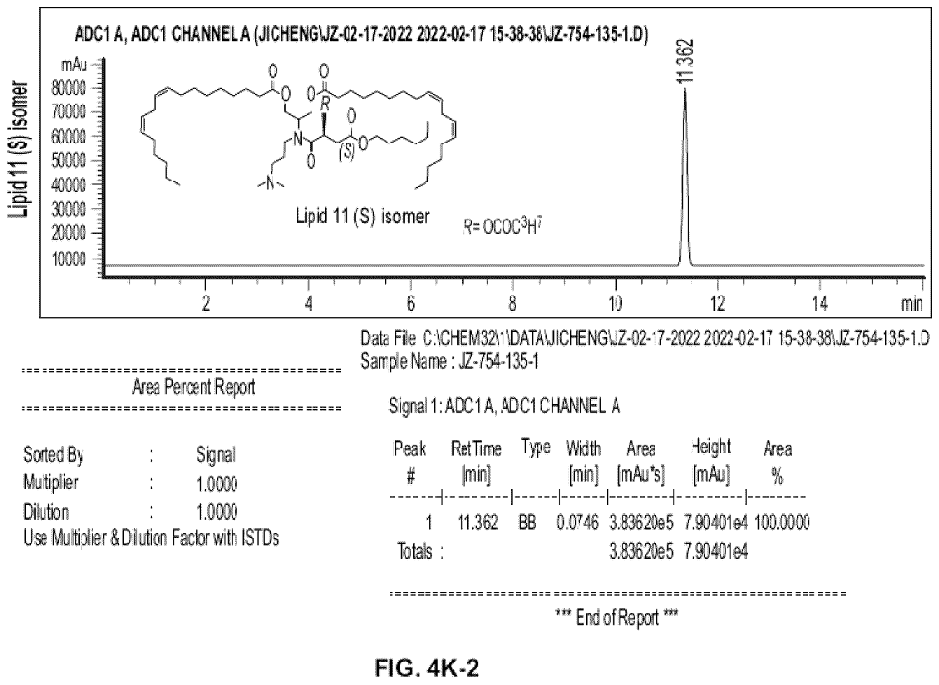

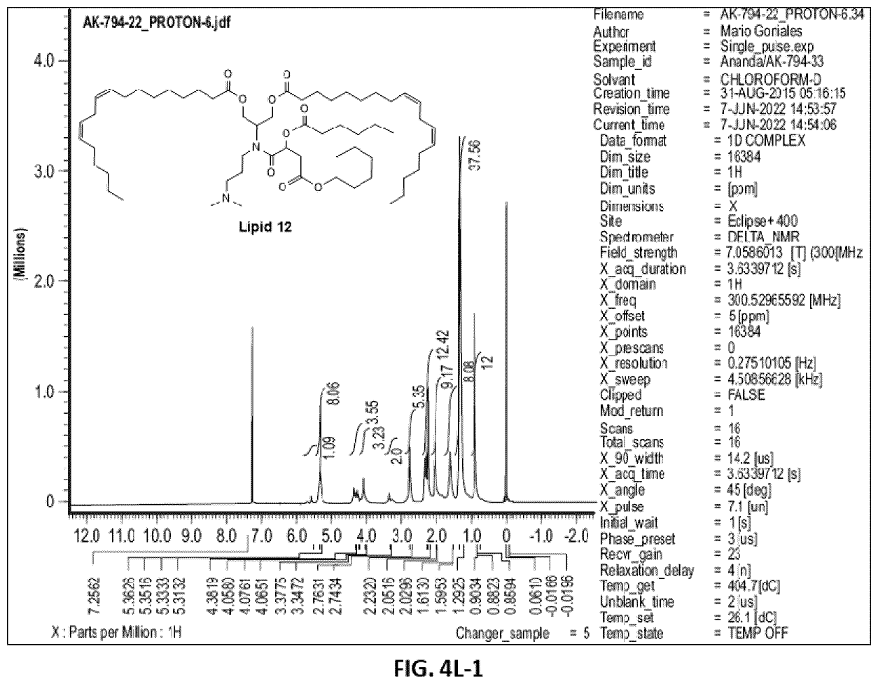

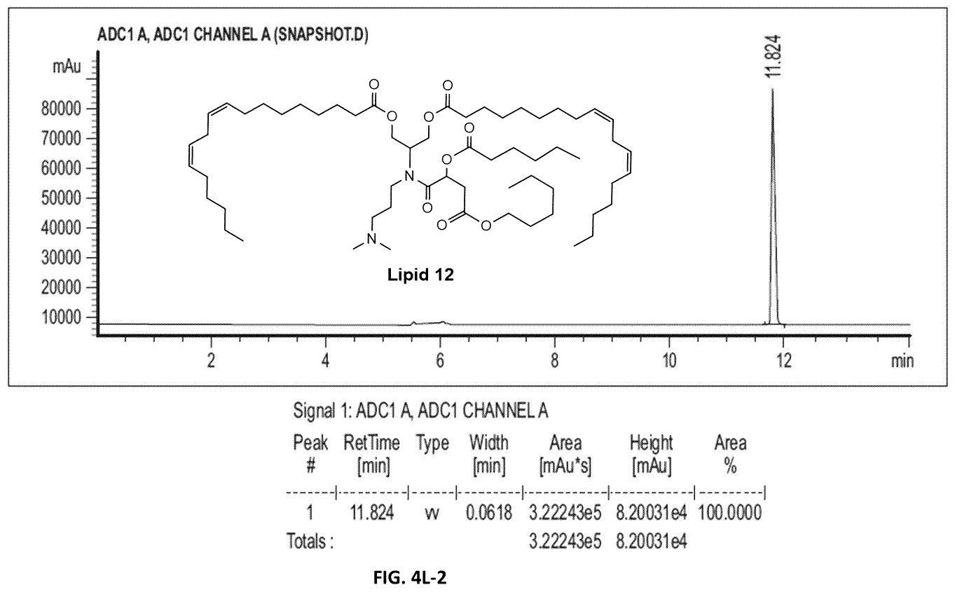

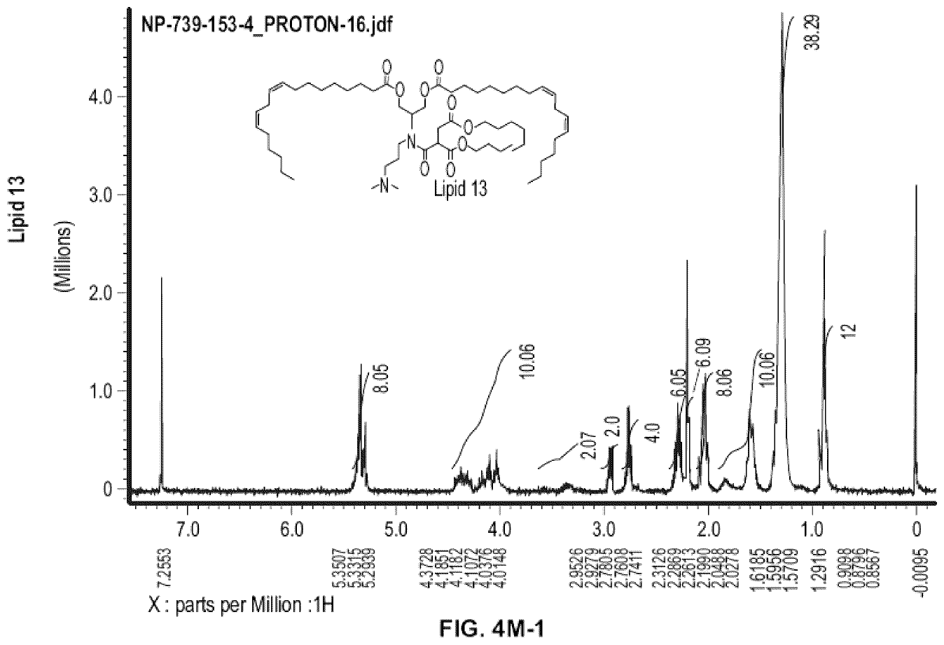

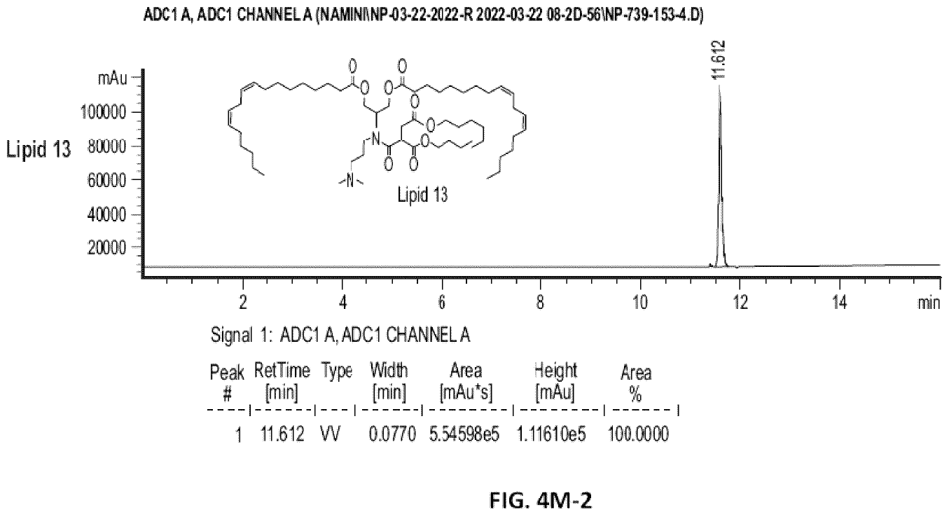

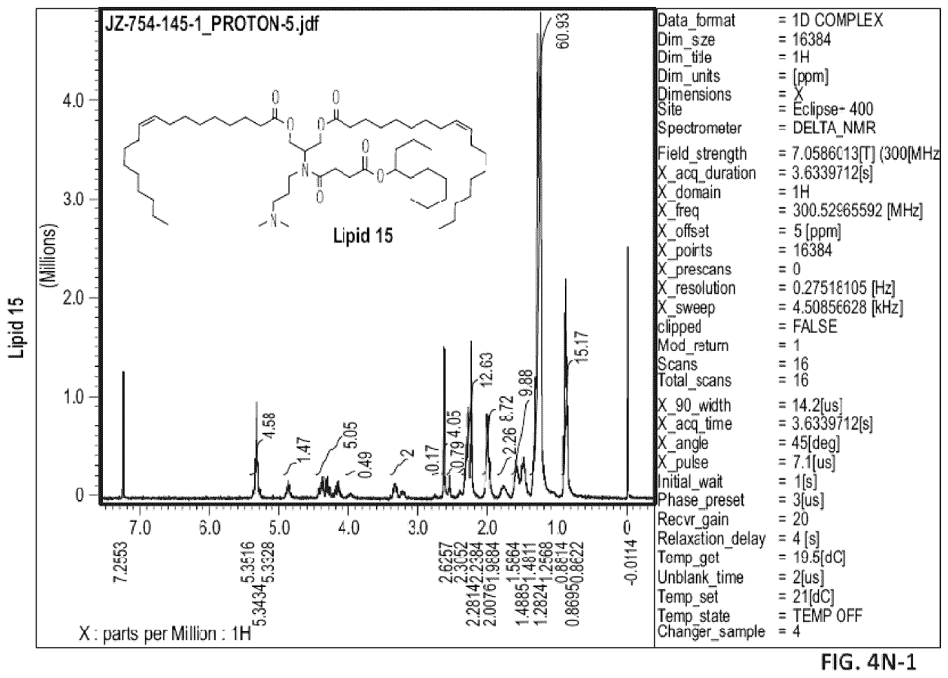

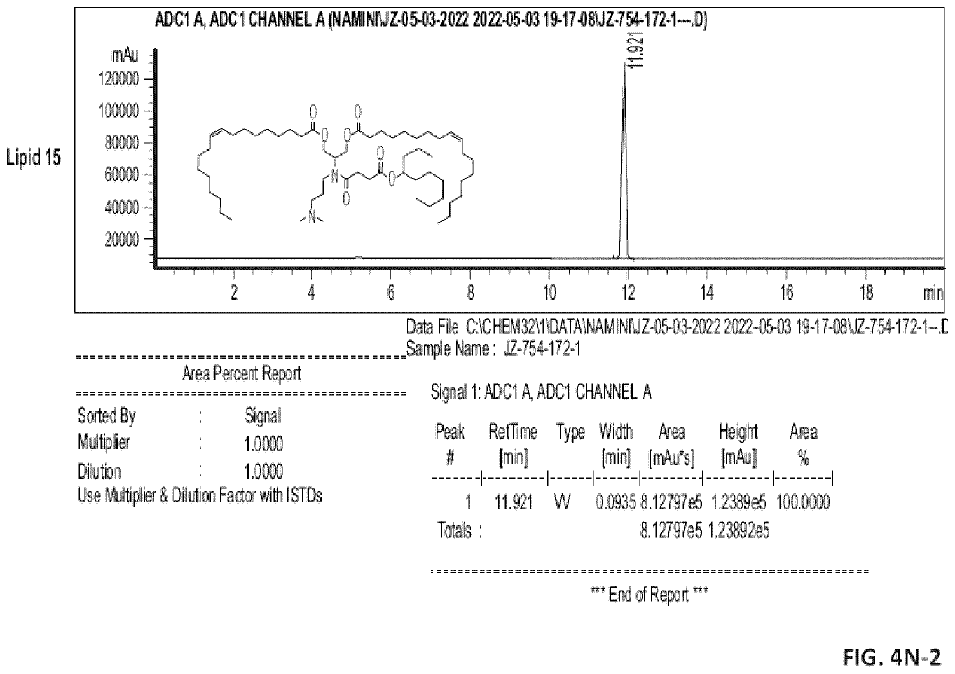

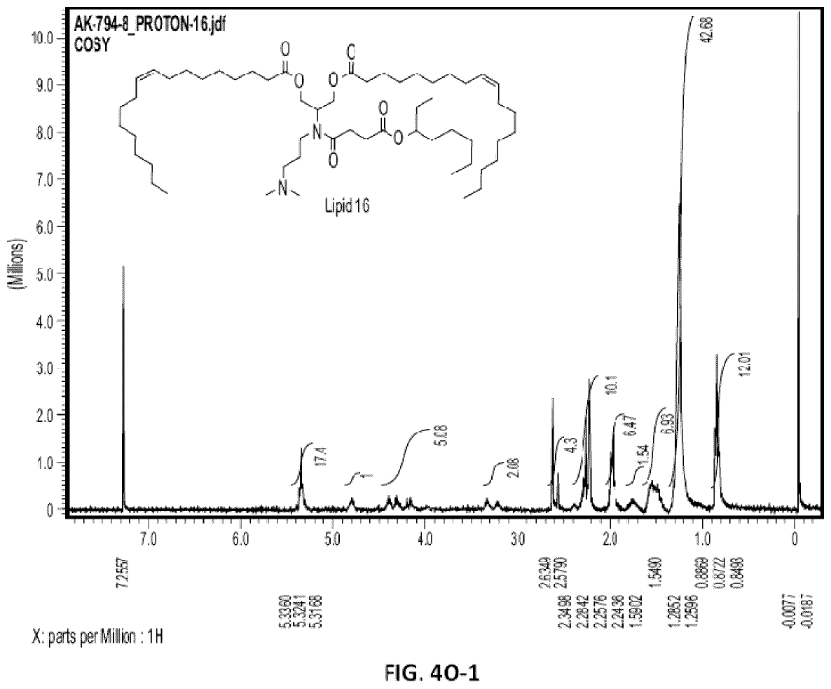

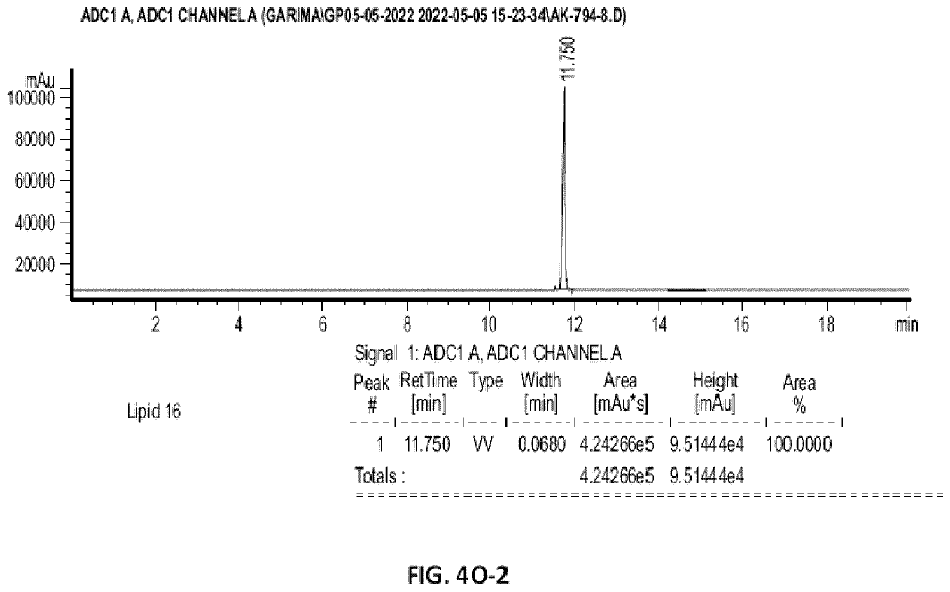

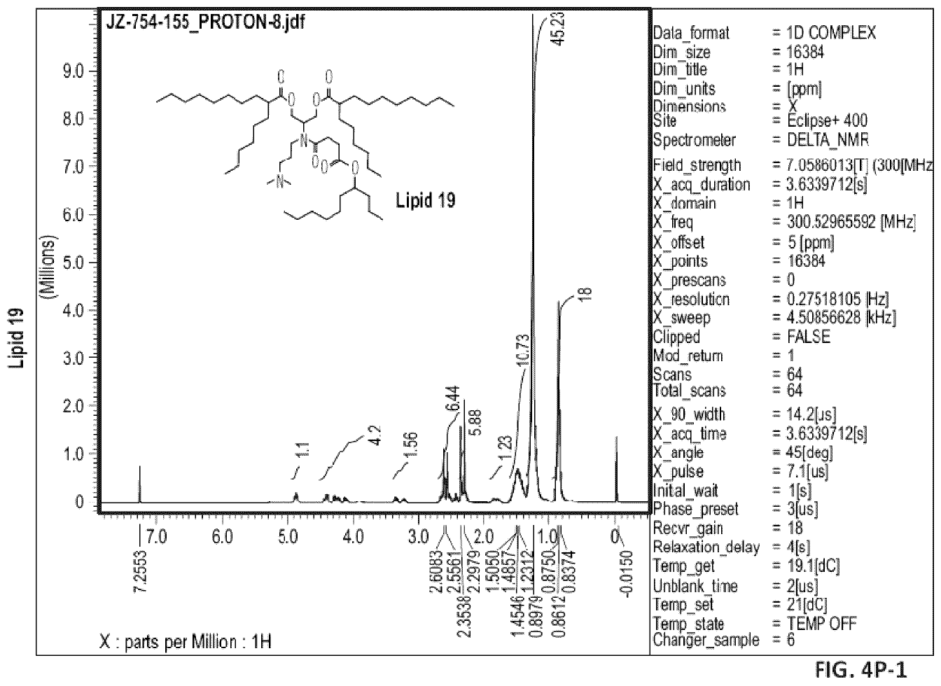

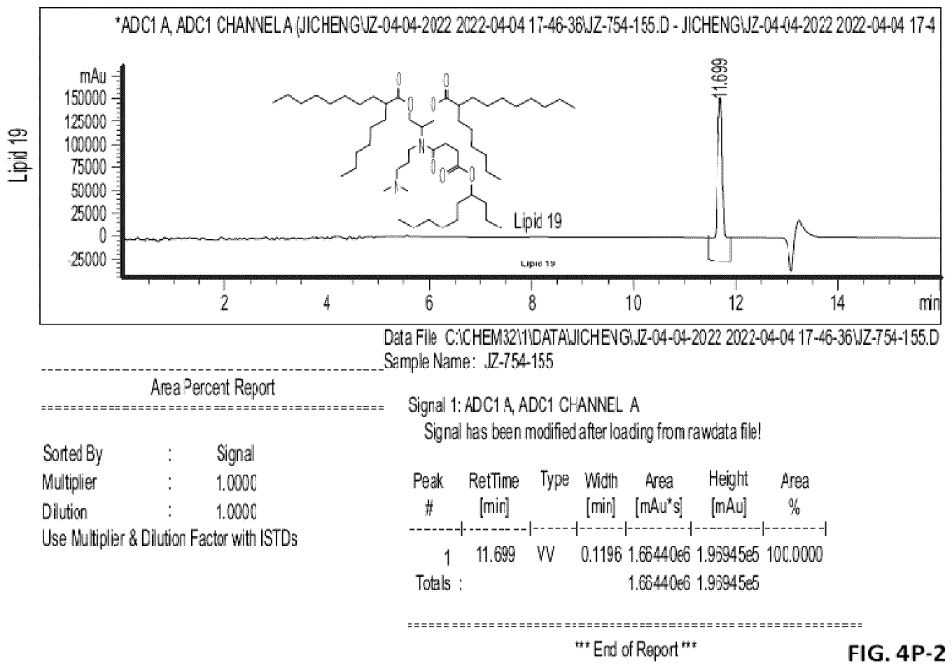

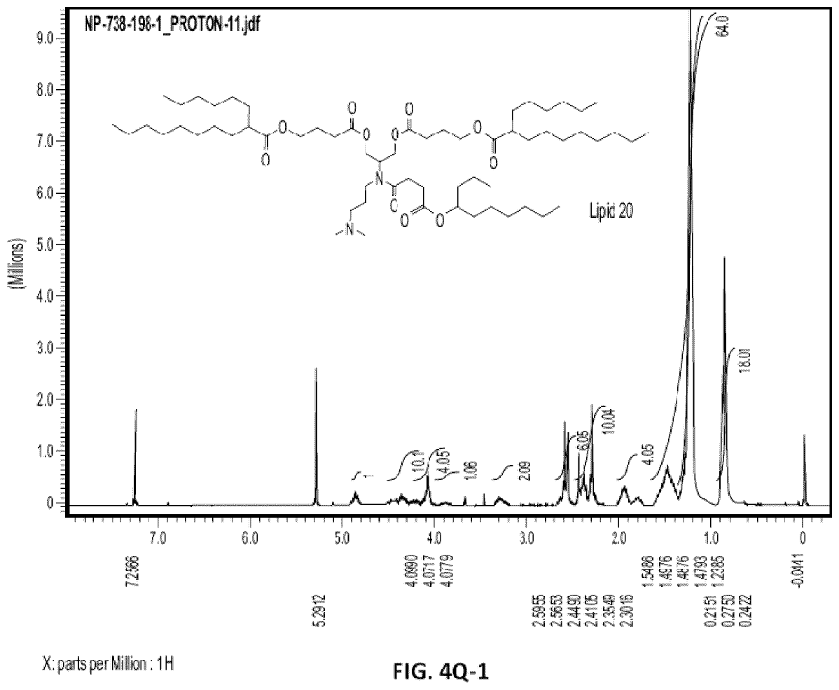

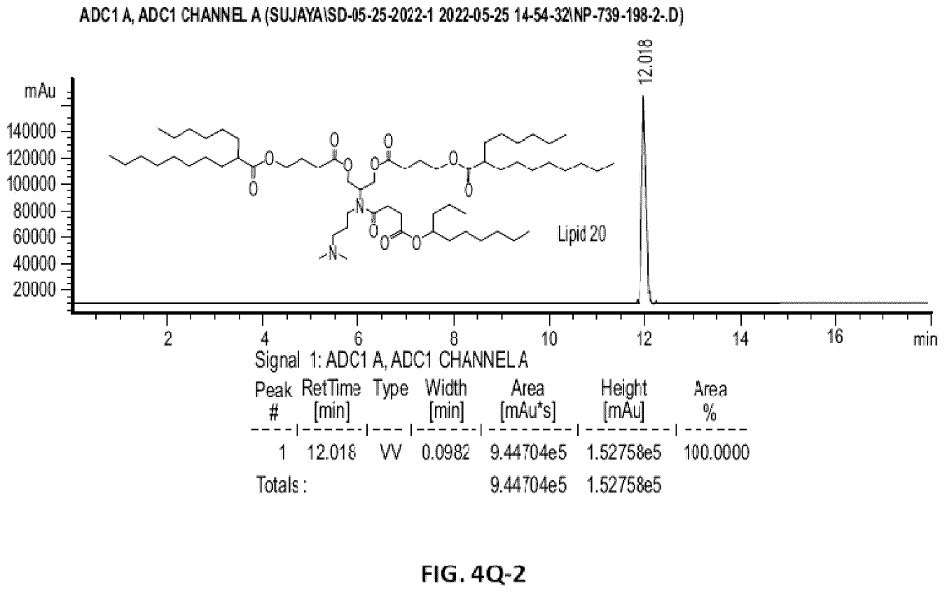

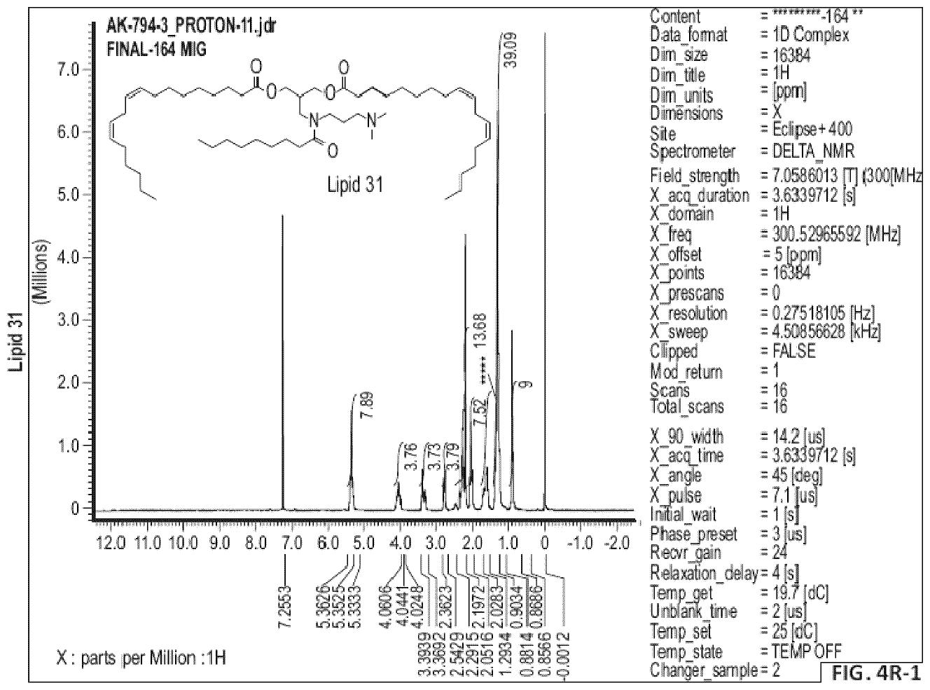

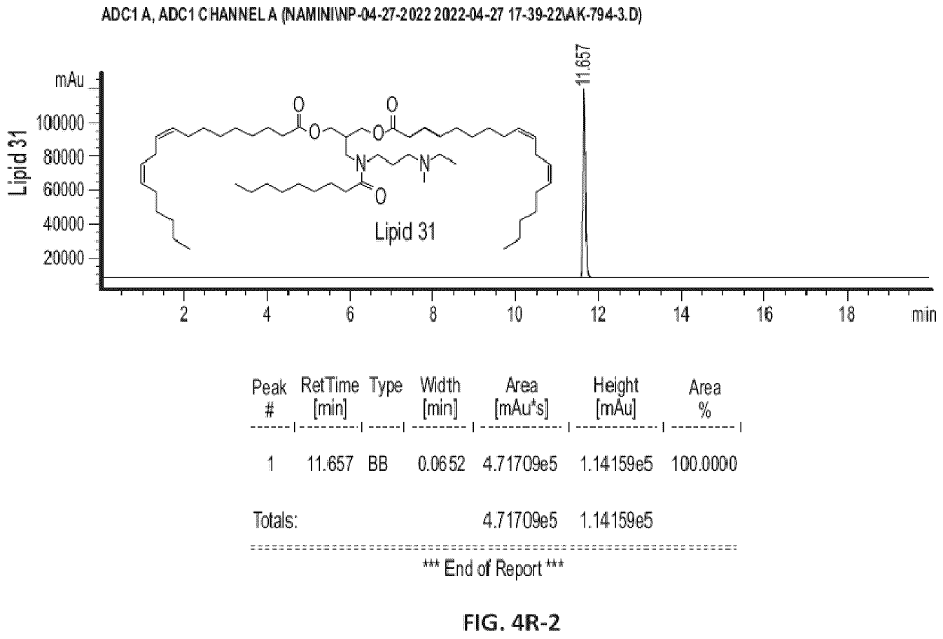

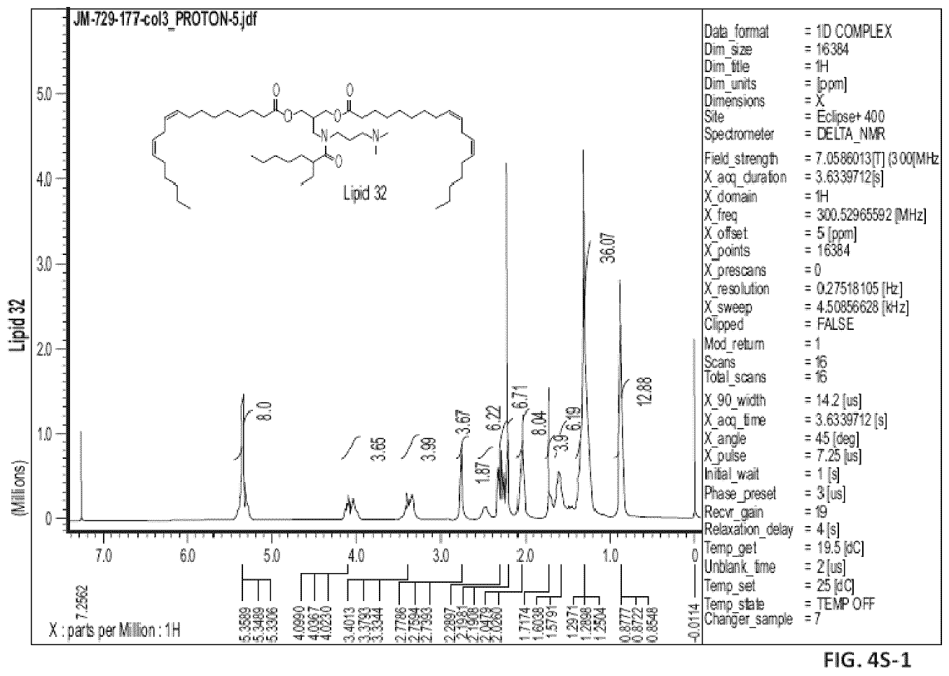

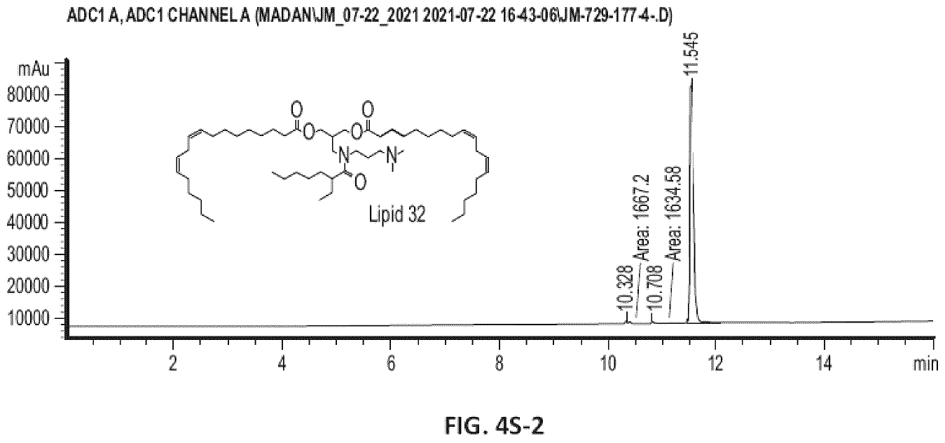

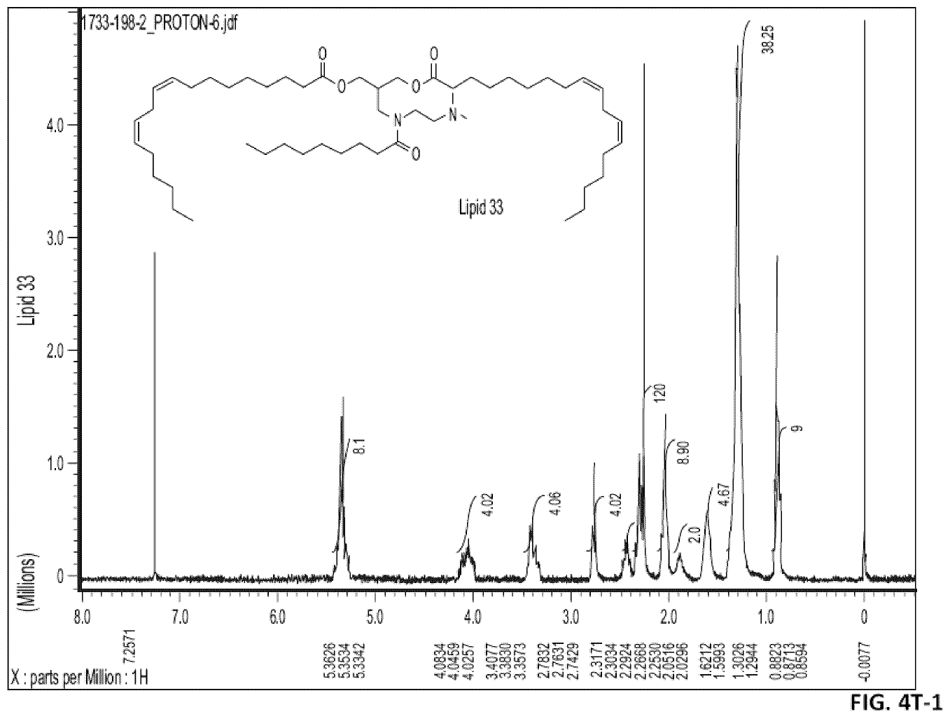

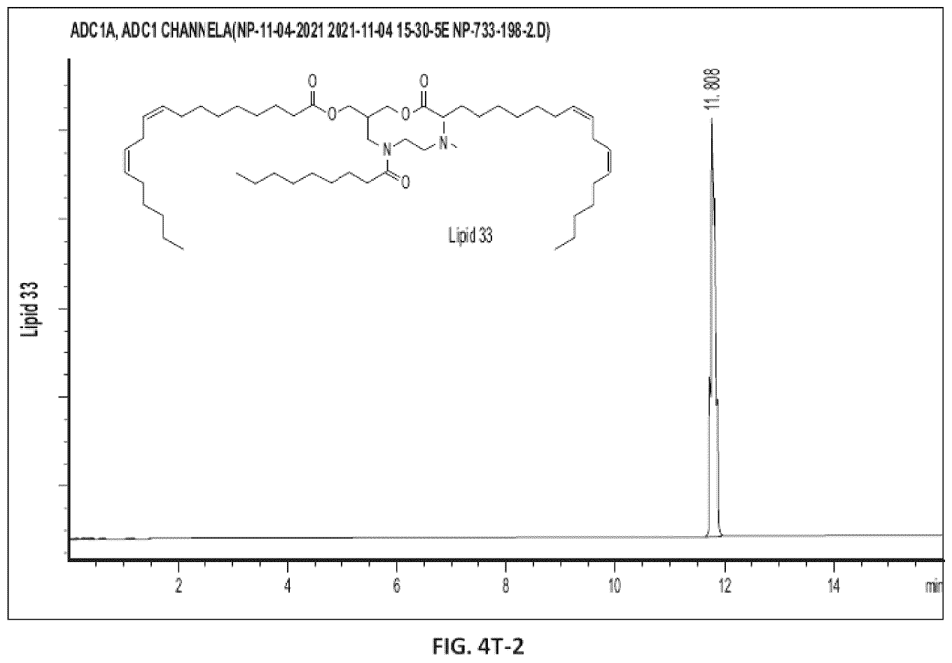

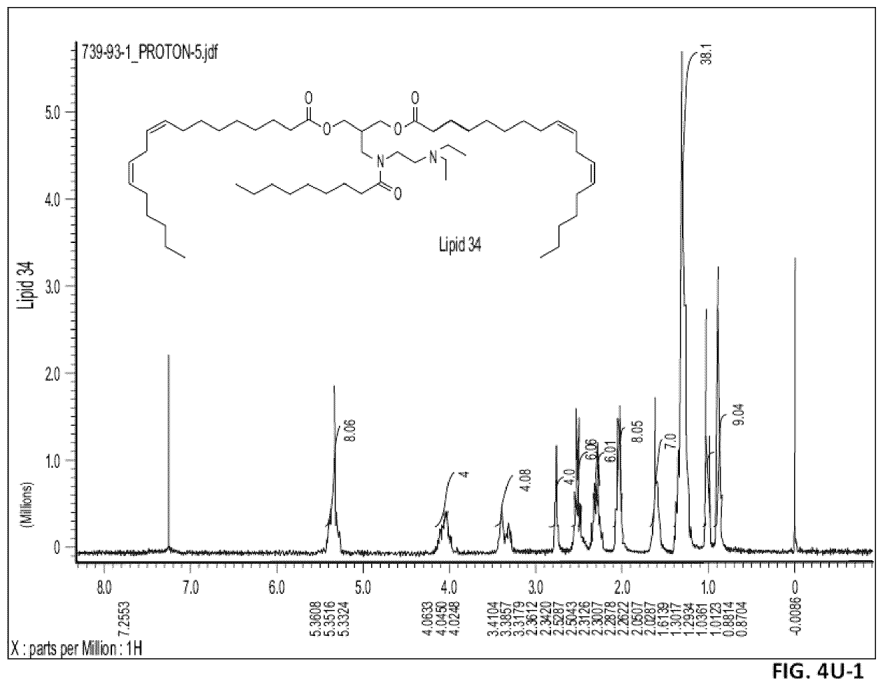

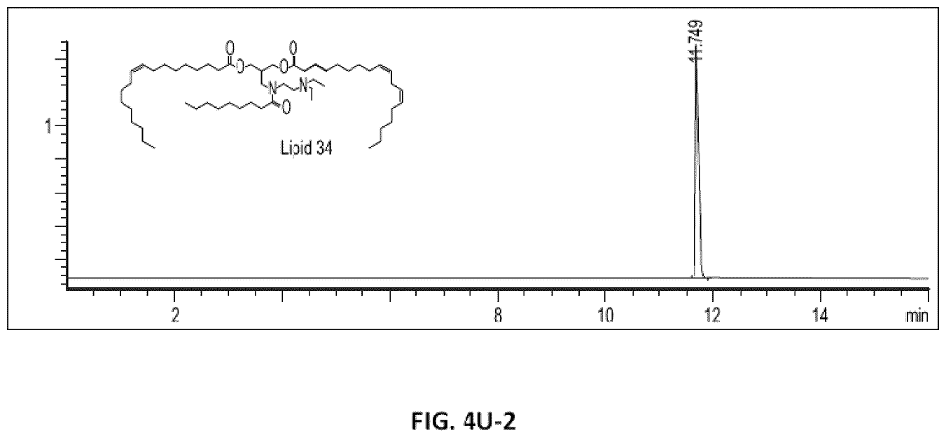

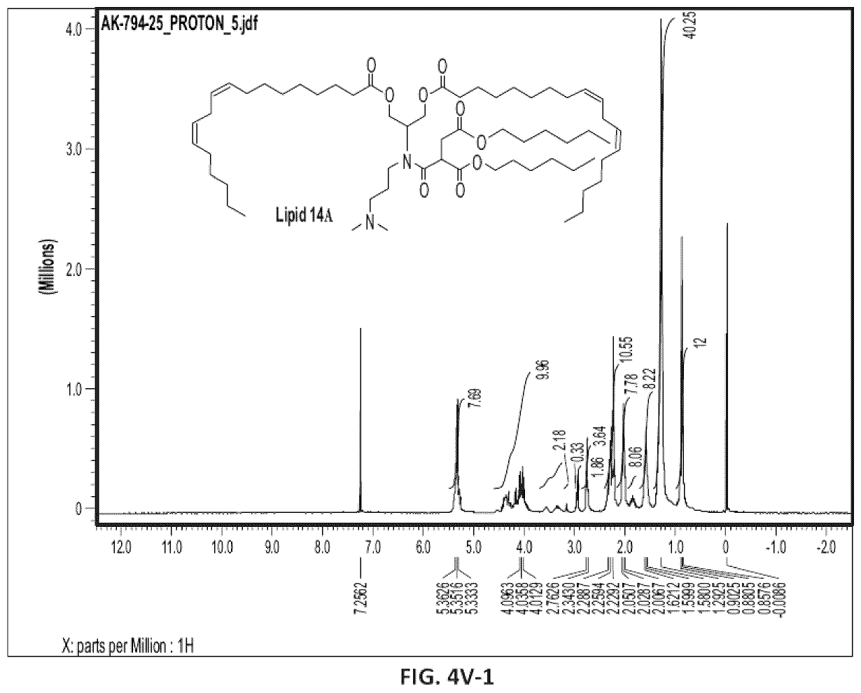

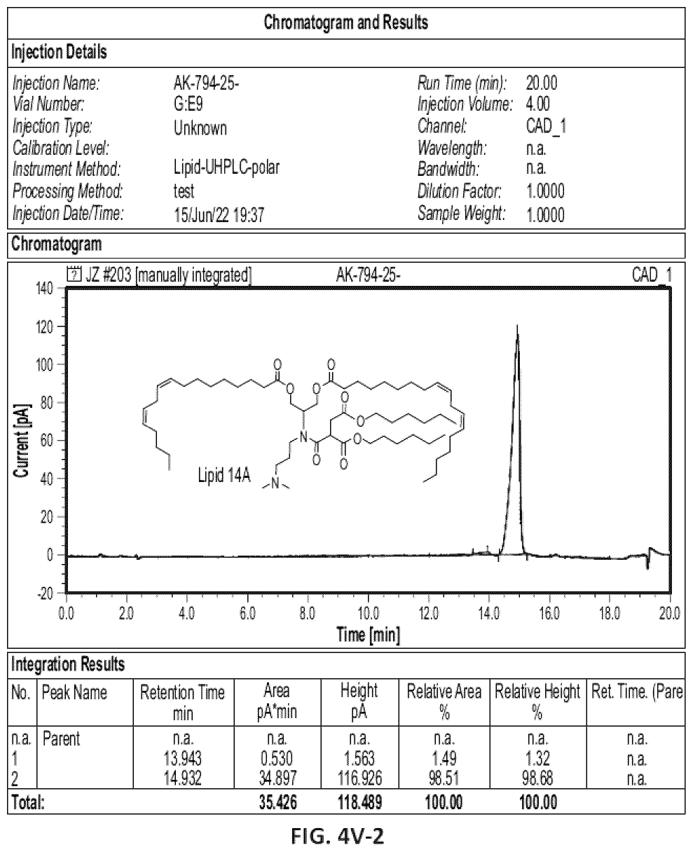

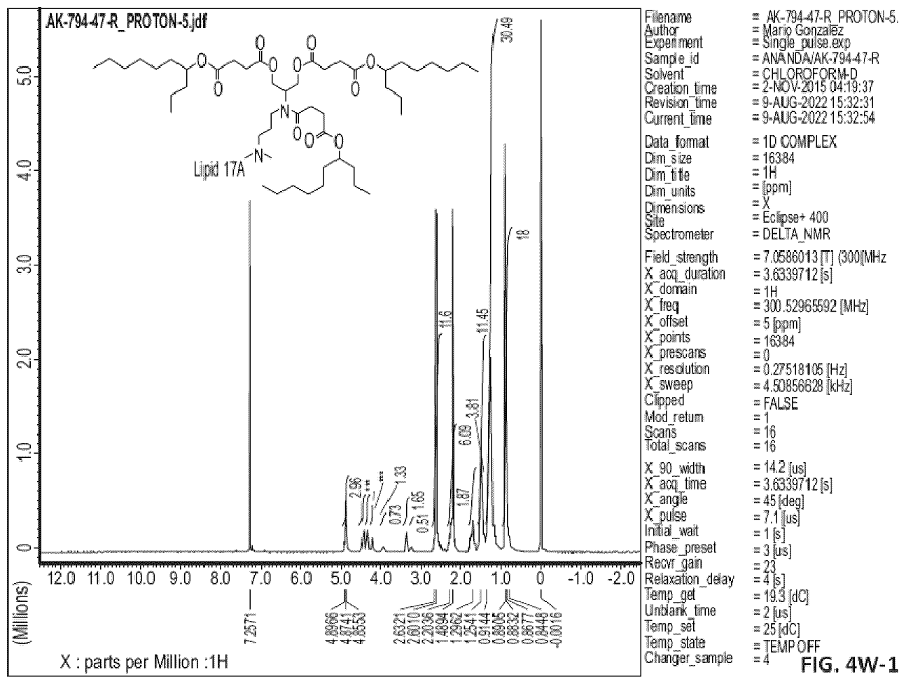

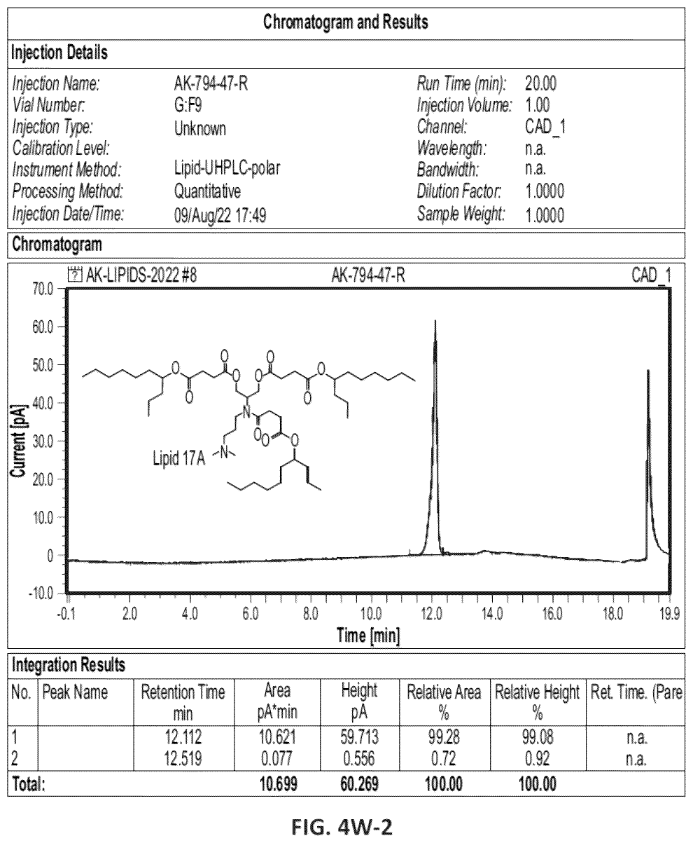

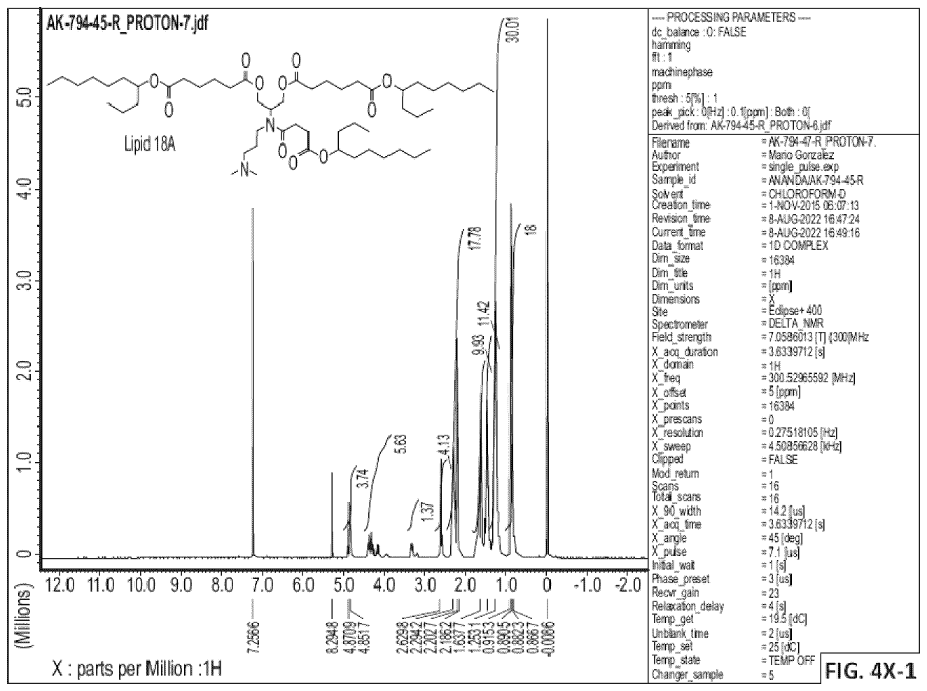

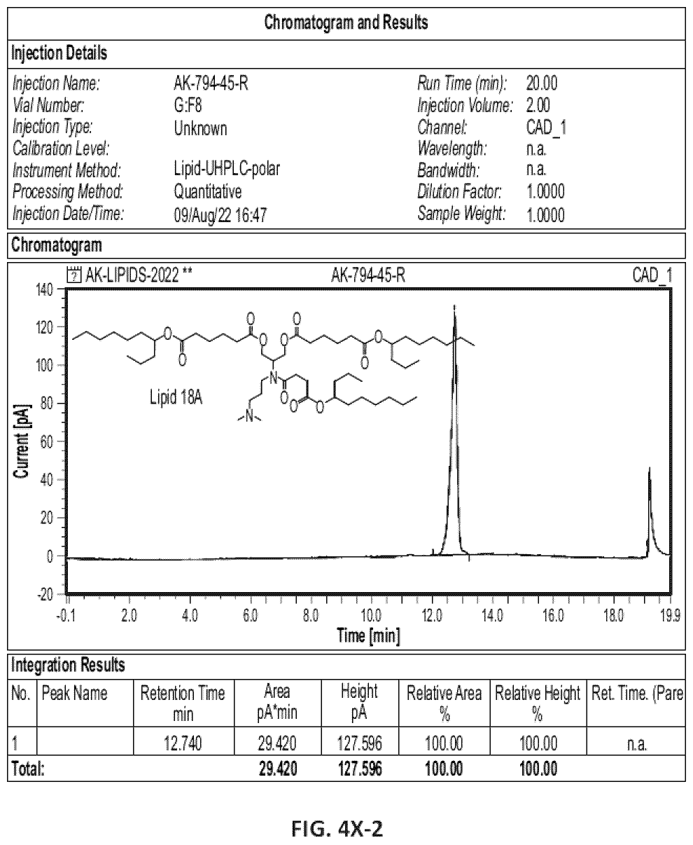

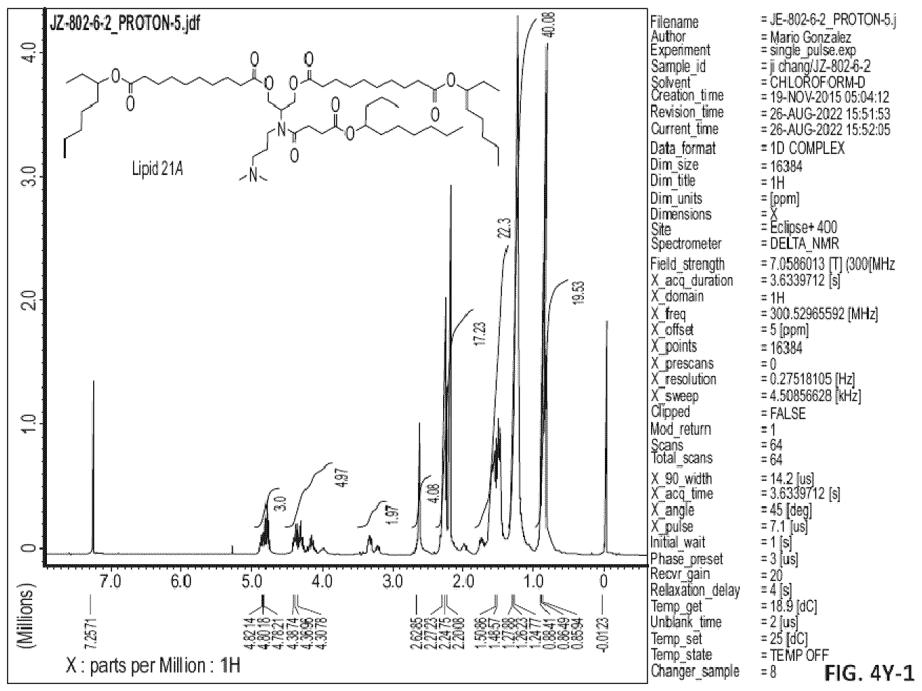

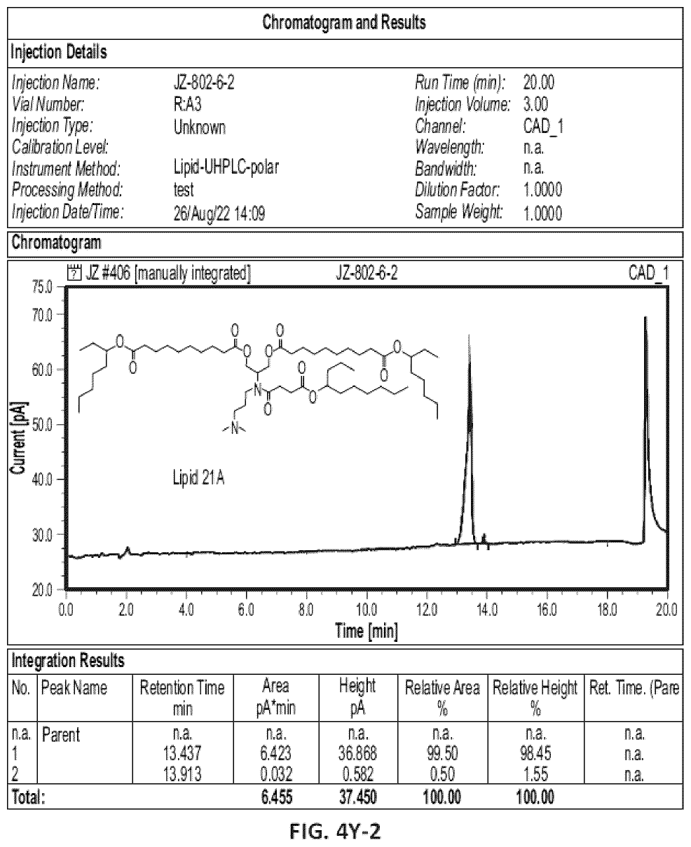

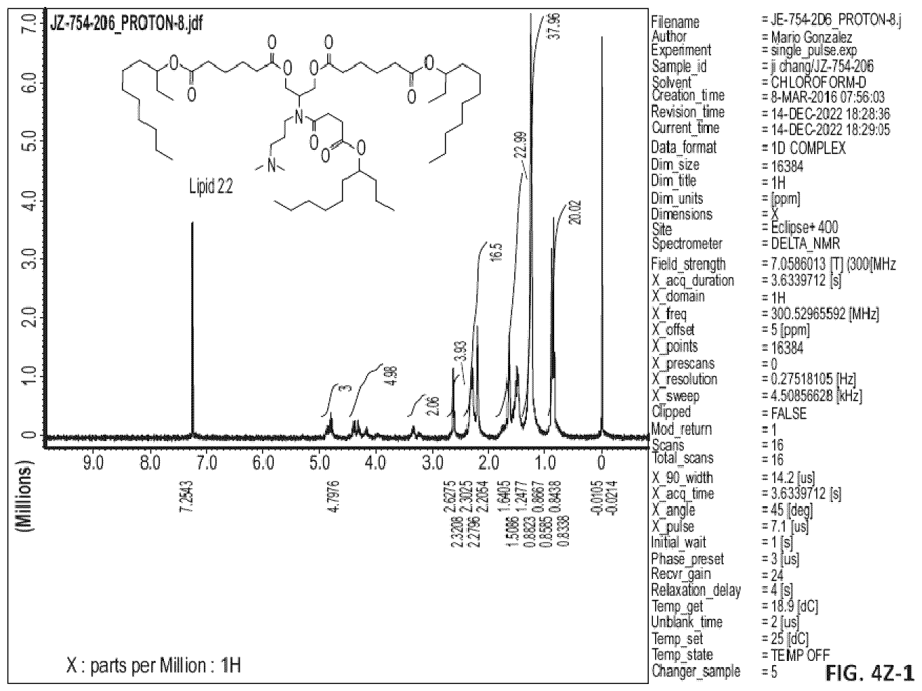

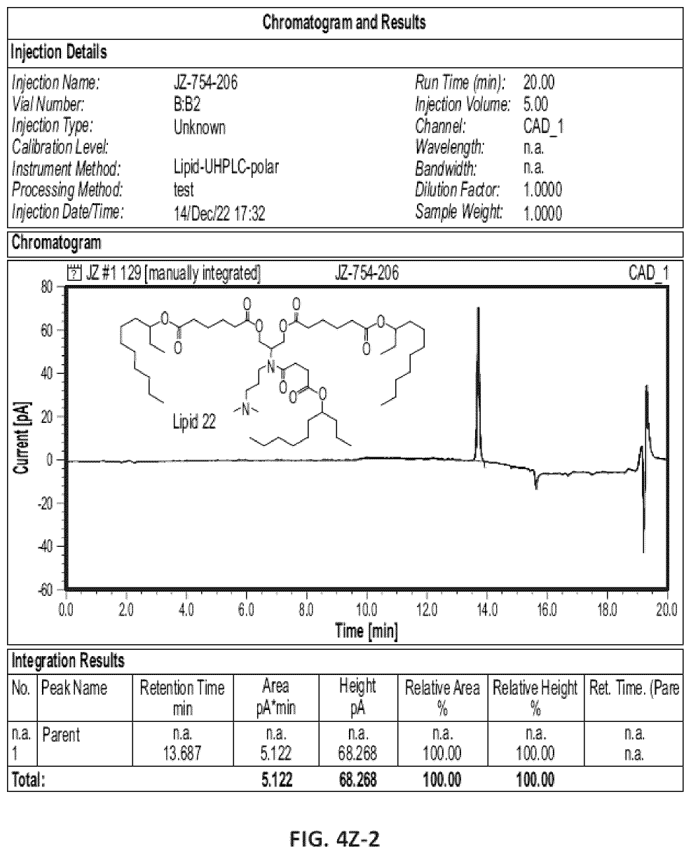

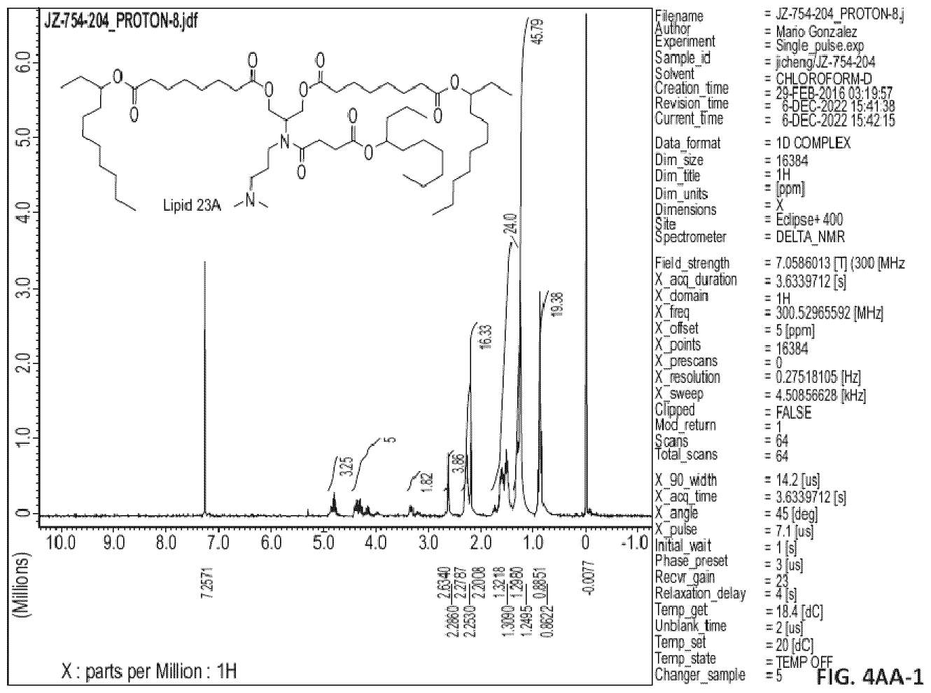

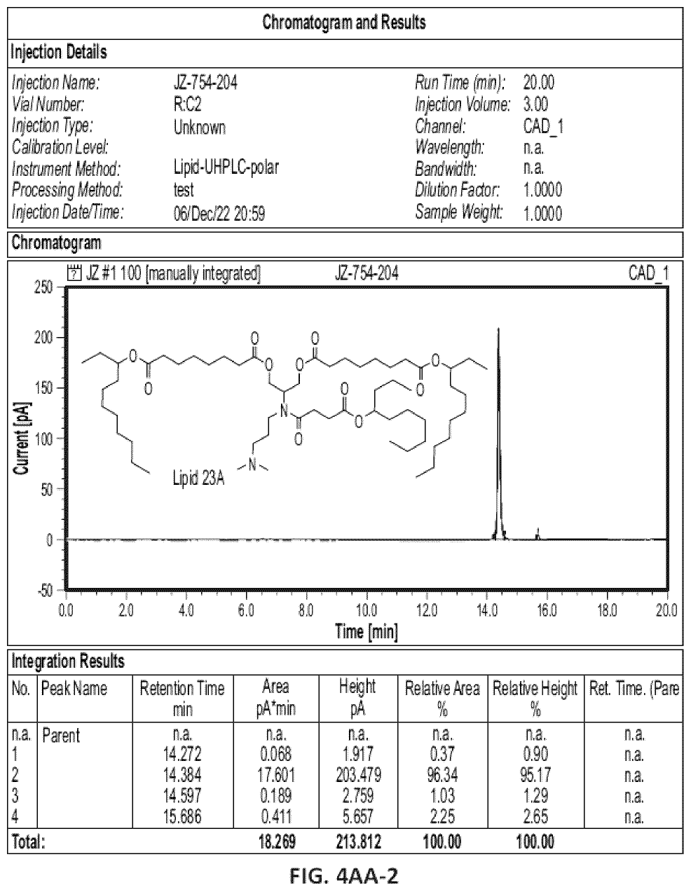

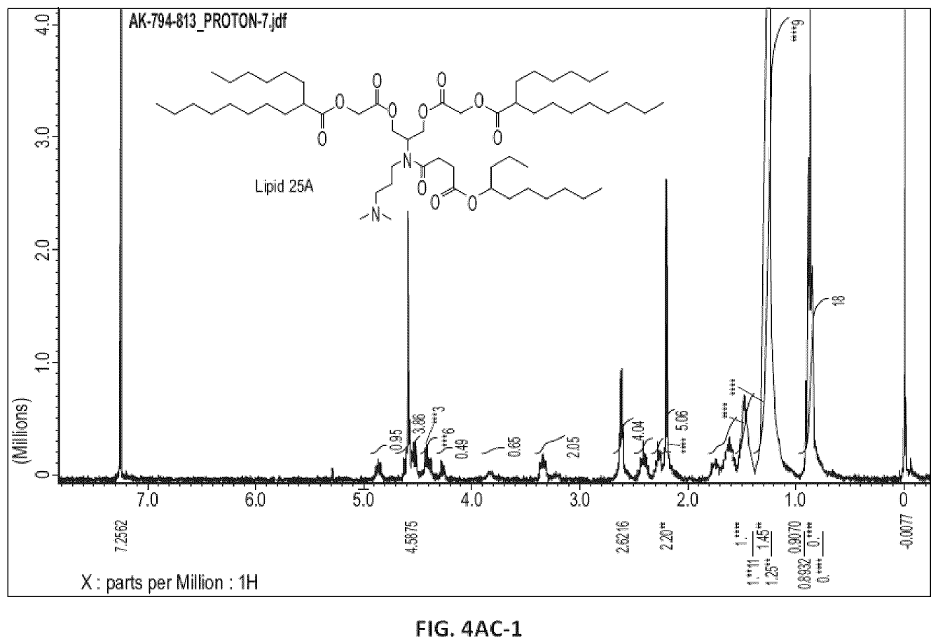

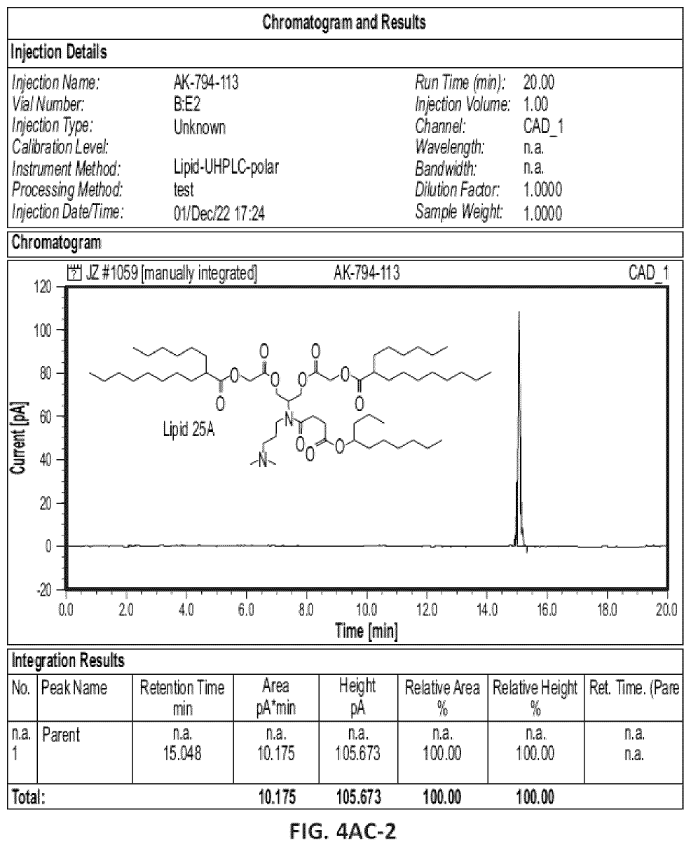

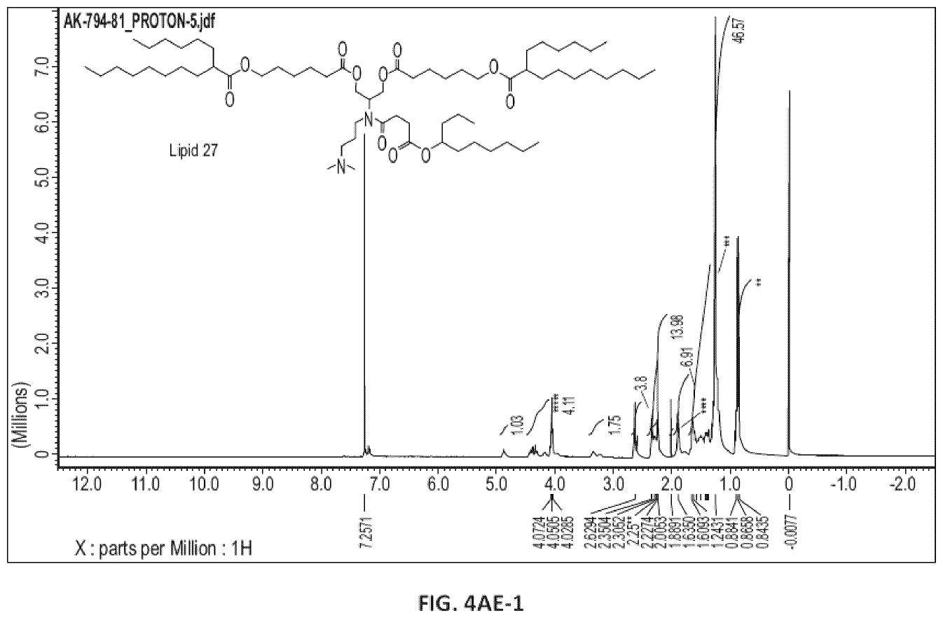

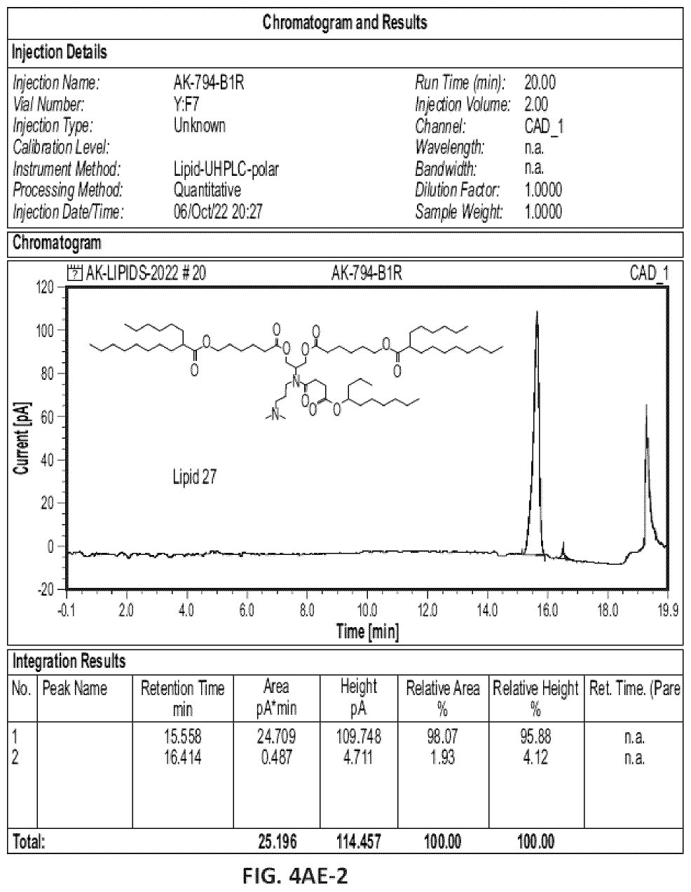

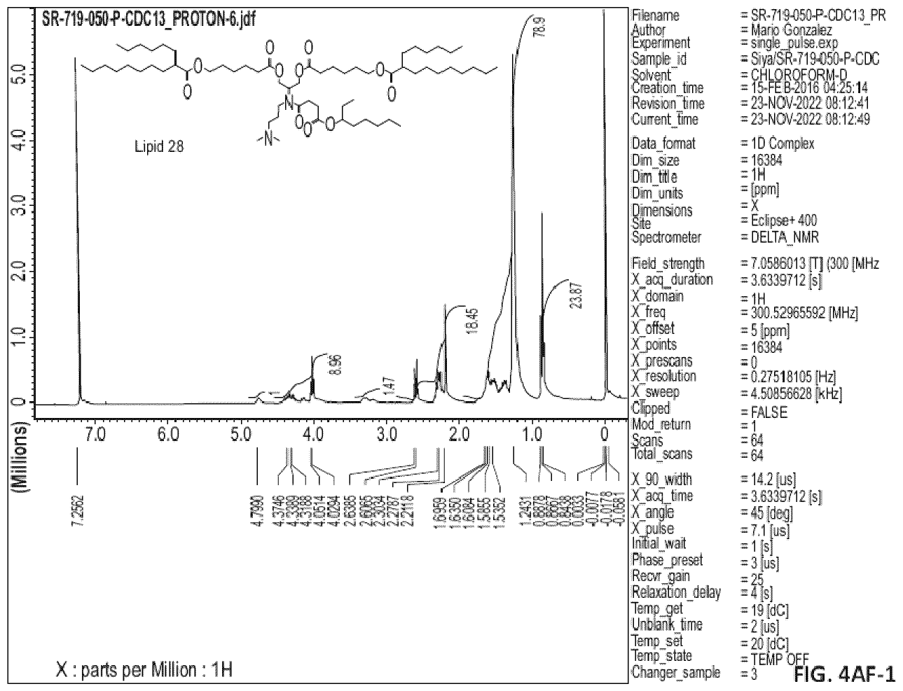

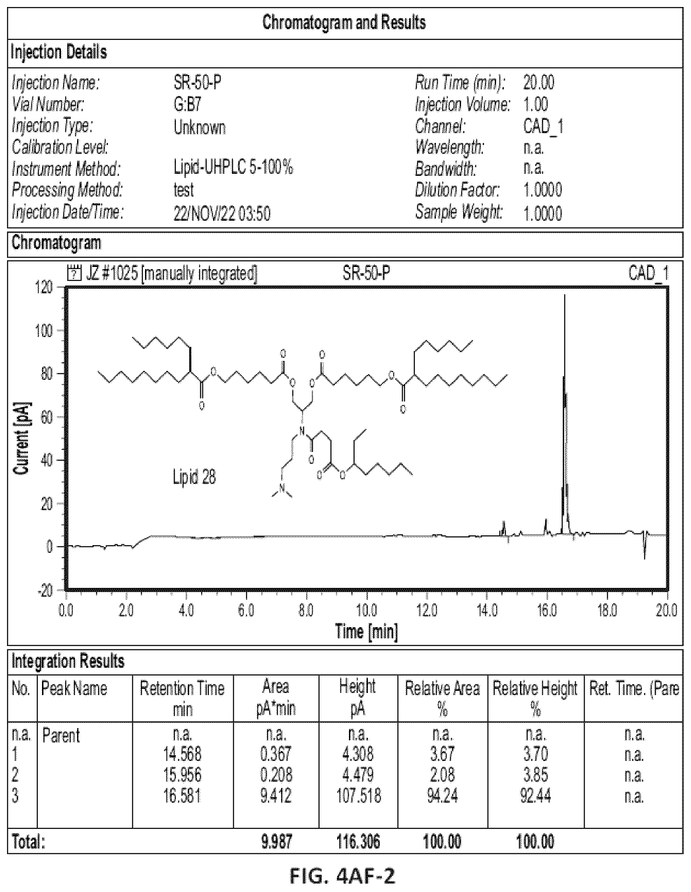

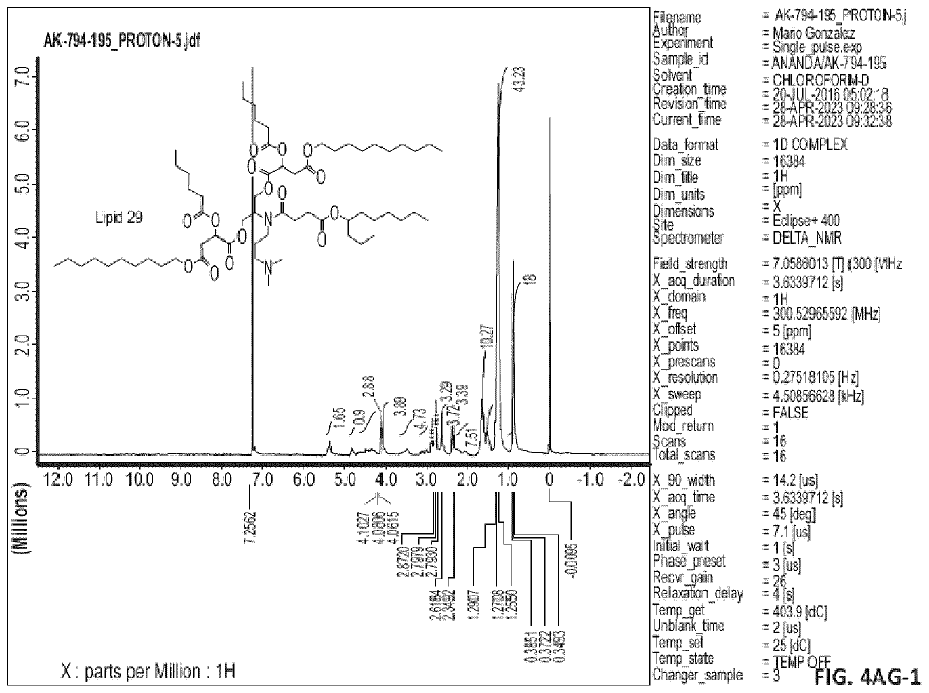

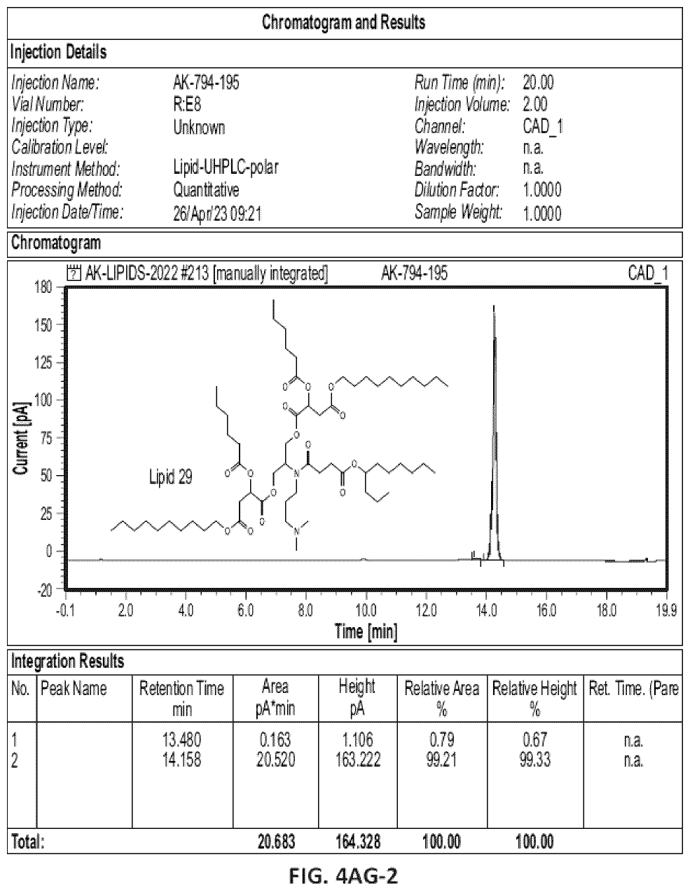

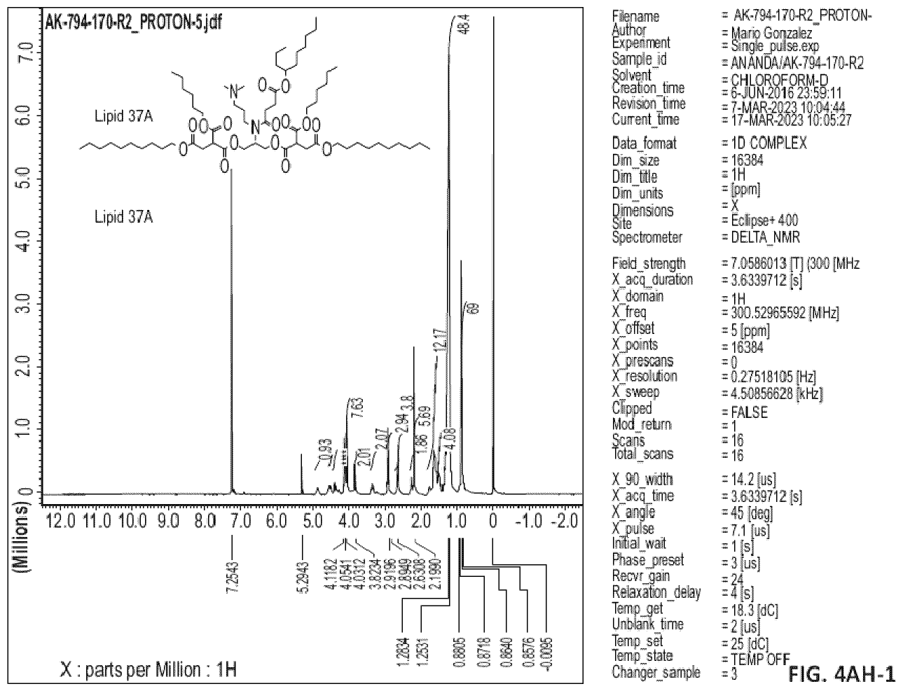

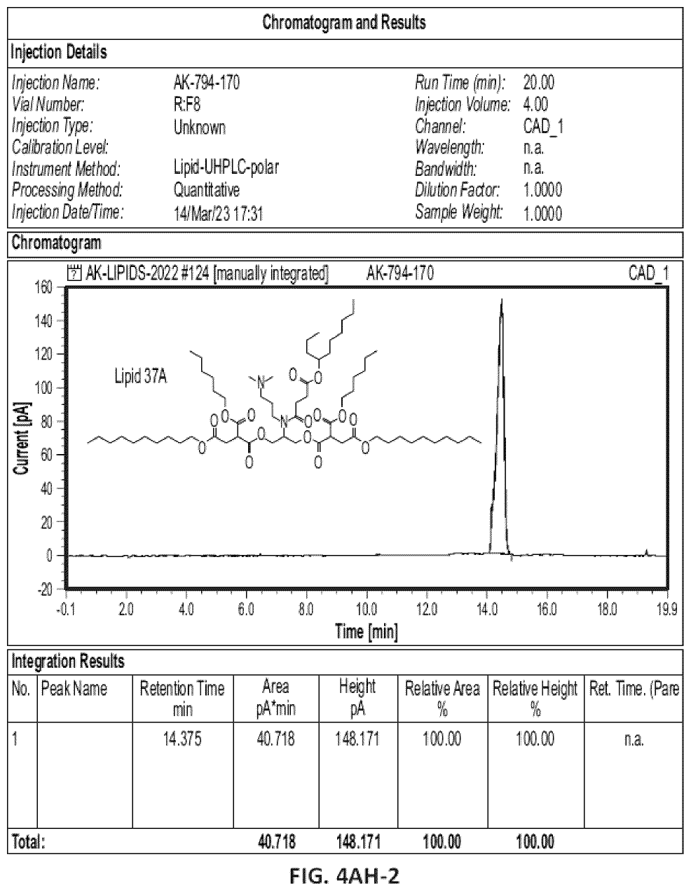

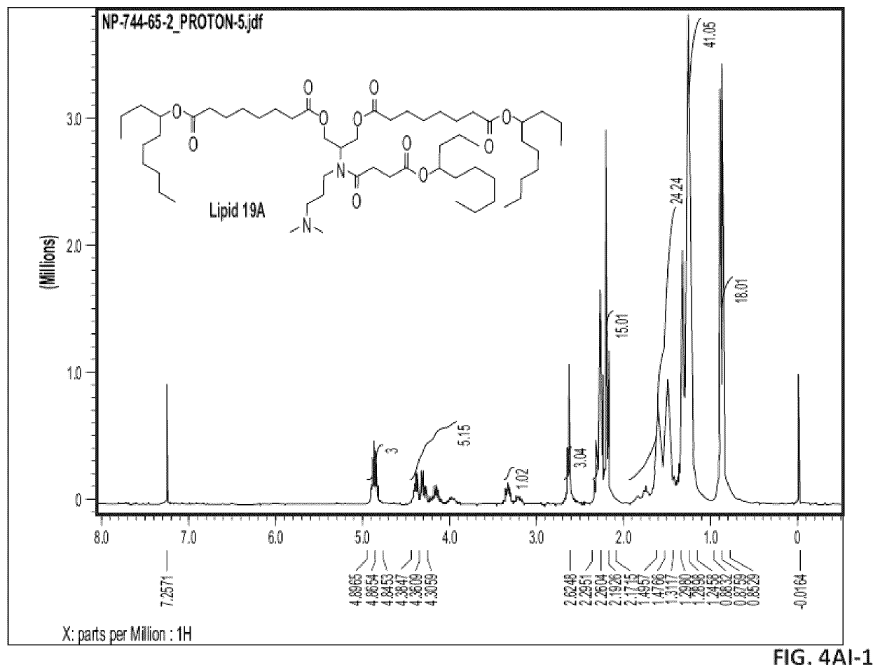

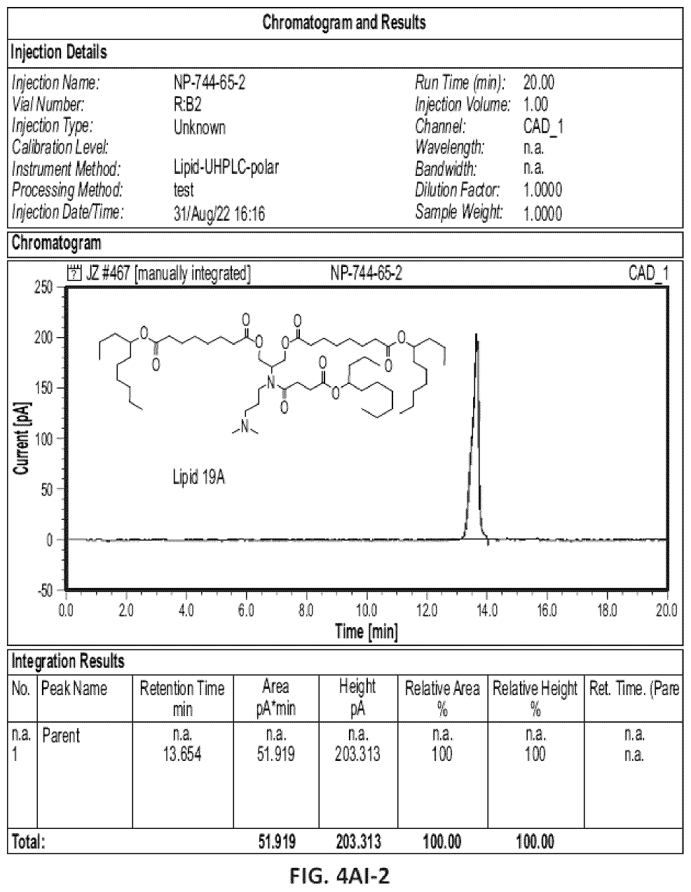

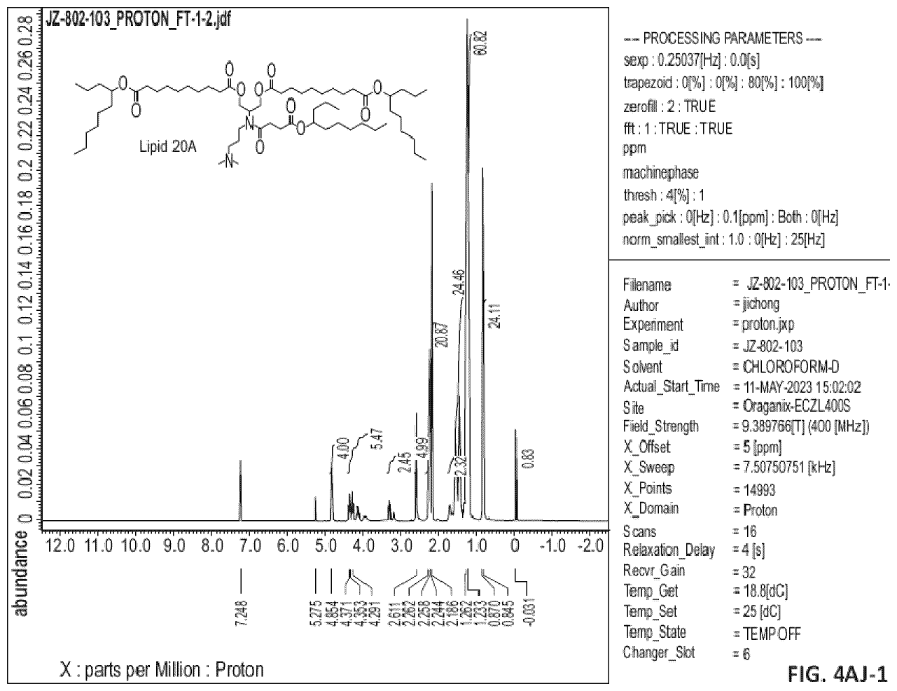

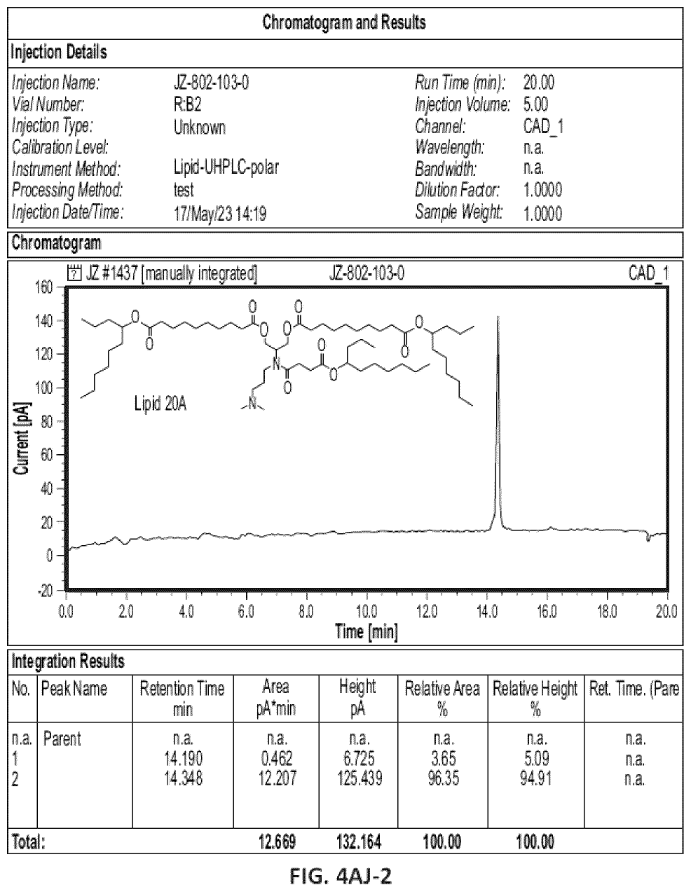

Lipid 1 was synthesized as provided in scheme 6 below and as follows. Starting material, 131-1 (0.75 mmol, 130 mg, 1.0 eq) was converted to the acid chloride (Step 1) using oxalyl chloride (3.7 mmol, 320 μl, 5 eq) and DMF (10 μl, catalytic) in 6 mL of benzene. Product (143 mg, 98%) showed only one spot on TLC (as methyl ester) and was used without further purification for acylation (step 2) of intermediate 13-0. Intermediate 13-0 (0.35 mmol, 250 mg, 1.0 eq.) was acylated with crude acid chloride, 131-1 (0.75 mmol, 143 mg, 1.7 eq.) using TEA (240 μL, 5 eq, 1.8 mmol) and DMAP (10 mg, catalytic amount). Crude product was purified by column chromatography (2×) yielding 124 mg (76%) of pure Lipid 1 (≥99% purity by LC-ELSD) and characterized by proton NMR and Mass Spectrometry (see FIG. 4A-1 for Lipid 1 NMR spectrum, and Table 5 for product mass).

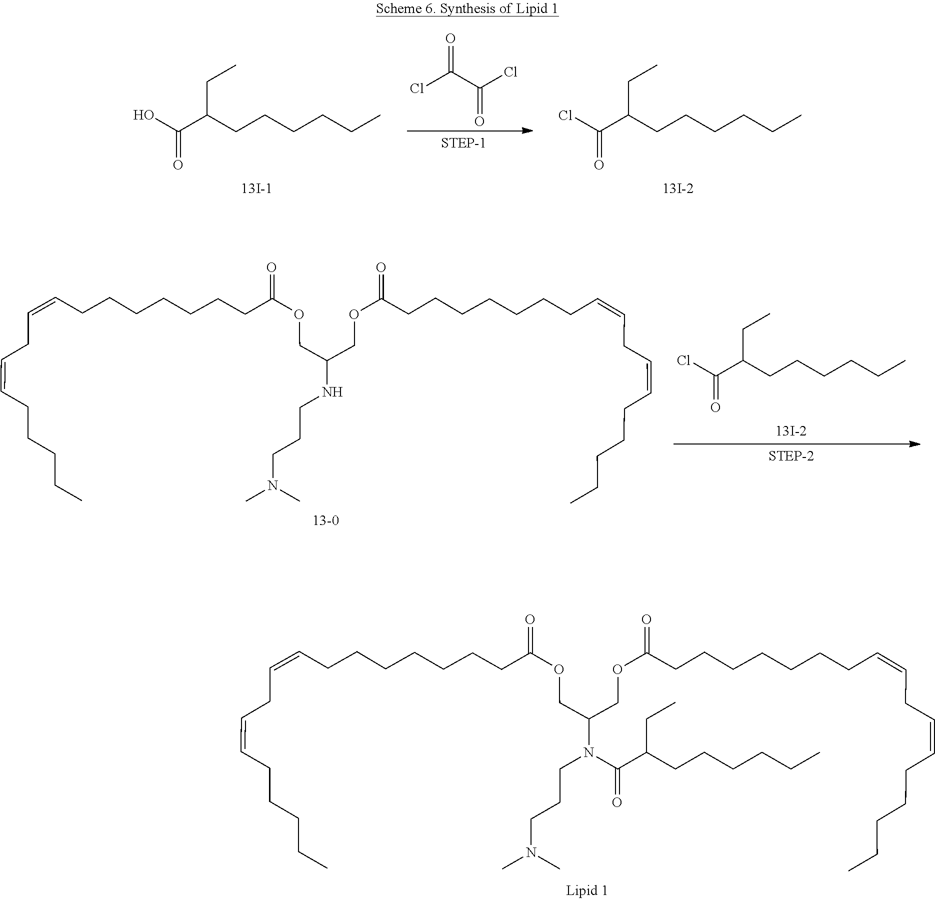

### Synthesis of Lipid 3

Lipid 3 was synthesized as provided in scheme 7 below and as follows. Starting material, 13-13 (8.3 mmol, 1.30 g, 1.0 eq) was converted to the acid chloride, 13-13a (Step 1) using oxalyl chloride (2.8 mmol, 2.4 ml, 5 eq.) and DMF (100 μl, catalytic) in 60 mL of benzene. Product (1.44 g, 98%) showed only one spot on TLC (as methyl ester) and was used without further purification for acylation (step 2) of intermediate 13-0. Intermediate 13-0 (5.4 mmol, 3.78 g, 1.0 eq.) was acylated with crude acid chloride, 13-13a (1.44 g, 1.5 eq, 8.1 mmol) using TEA (3.76 mL, 5 eq, 27 mmol) and DMAP (50 mg, cat, catalytic amount) in benzene (100 mL). Crude product was purified by column chromatography (2×) yielding 2.1 g (46.3%) of pure Lipid 3 (≥99% purity by LC-ELSD) and characterized by proton NMR and Mass Spectrometry (see FIG. 4B-1 for Lipid 3 NMR spectrum, FIG. 4B-2 for Lipid 3 LC-MS, and Table 5 for product mass).

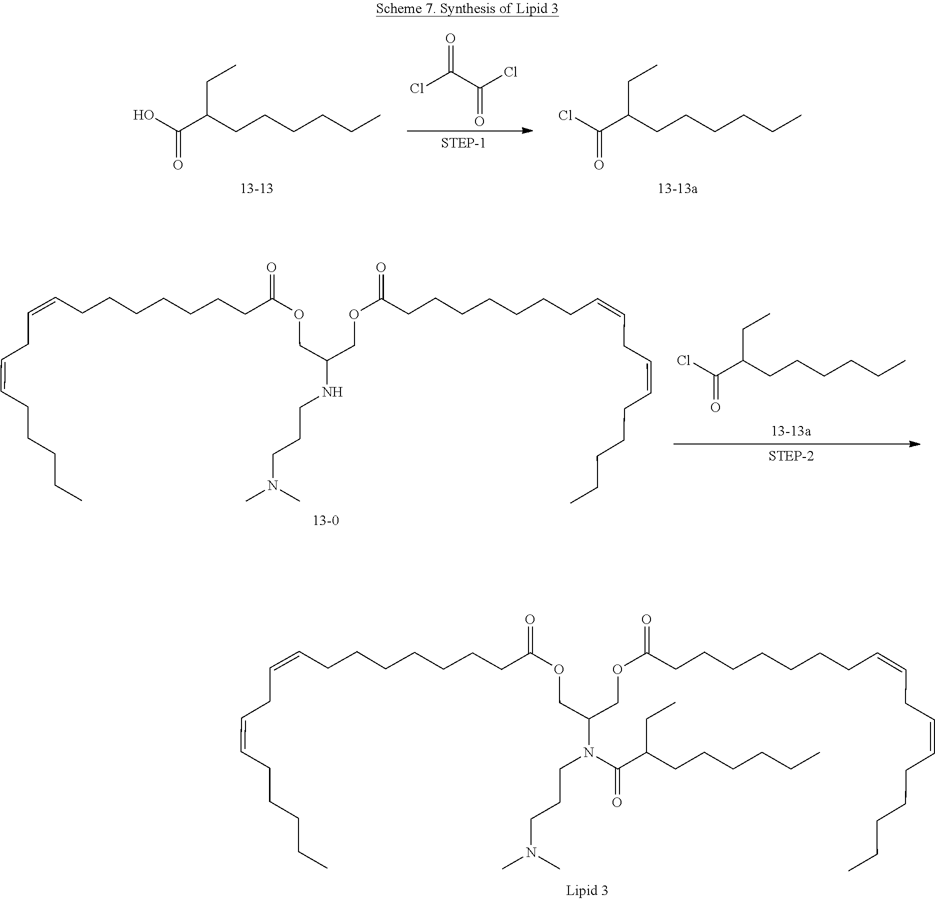

### Synthesis of Lipid 4

Lipid 4 was synthesized as provided in scheme 7 below and as follows. Starting material, 13-18 (0.95 mmol, 150 mg, 1 eq.) was converted to the acid chloride, 13-18′ (Step 1) using oxalyl chloride (3.23 mmol, 227 μl, 3.4 eq.) and DMF (10 μl, catalytic) in 6 mL of benzene. Product showed only one spot on TLC (as methyl ester) and was used without further purification for acylation (step 2) of intermediate 13-11b. Intermediate 13-11b (0.63 mmol, 444 mg, 1.0 eq.) was acylated with crude acid chloride, 13-18′ (167 mg, 1.5 eq, 0.95 mmol) using TEA (445 μL, 5.0 eq, 3.2 mmol) and DMAP (10 mg, catalytic amount) in benzene (10 mL). Crude product was purified by column chromatography (5×) yielding 140 mg (26%) of pure Lipid 4 (97% purity by LC-ELSD) and characterized by proton NMR and Mass Spectrometry (see FIG. 4C-1 for Lipid 4 NMR spectrum, FIG. 4C-2 for Lipid 4 LC-MS, and Table 5 for product mass).

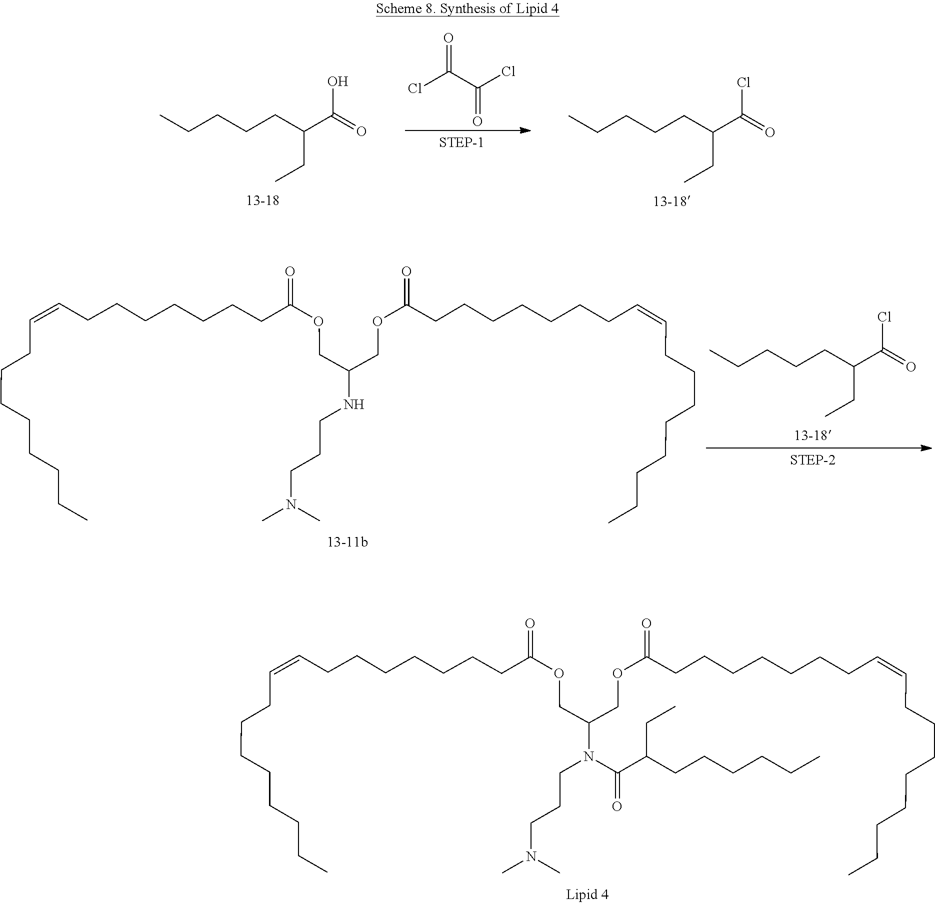

### Synthesis of Lipid 5 and its (S) isomer

The (S) isomer of lipid 5 was synthesized as provided in scheme 9-1 below and as follows. Starting material, Ethyl hexenoic acid 13 m-1 (110 mg, 1.0 eq, 0.75 mmol) was converted to the acid chloride, 13 m-2 (Step 1) using oxalyl chloride (320 μL, 1.0 eq, 3.7 mmol) and DMF (20 μl, catalytic) under reflux for 2 hours in 3 mL of benzene. Product showed only one spot on TLC (as methyl ester) and was used without further purification for acylation (step 2) of intermediate 13-0. Intermediate 13-0 (250 mg, 1.0 eq, 0.35 mmol) was acylated with crude acid chloride, 13 m-2 (120 mg, 1.8 eq, 0.75 mmol) using TEA (240 μL, 5.0 eq, 1.8 mmol) and DMAP (10 mg, catalytic amount) in 10 mL benzene, overnight at room temperature. Crude product was purified by column chromatography (2×) yielding 95 mg (32%) of pure Lipid 5 (≥99% purity by LC-ELSD) and characterized by proton NMR and Mass Spectrometry (see FIG. 4D-1 for Lipid 5 NMR spectrum, FIG. 4D-2 for Lipid 5 LC-MS, and Table 5 for product mass).

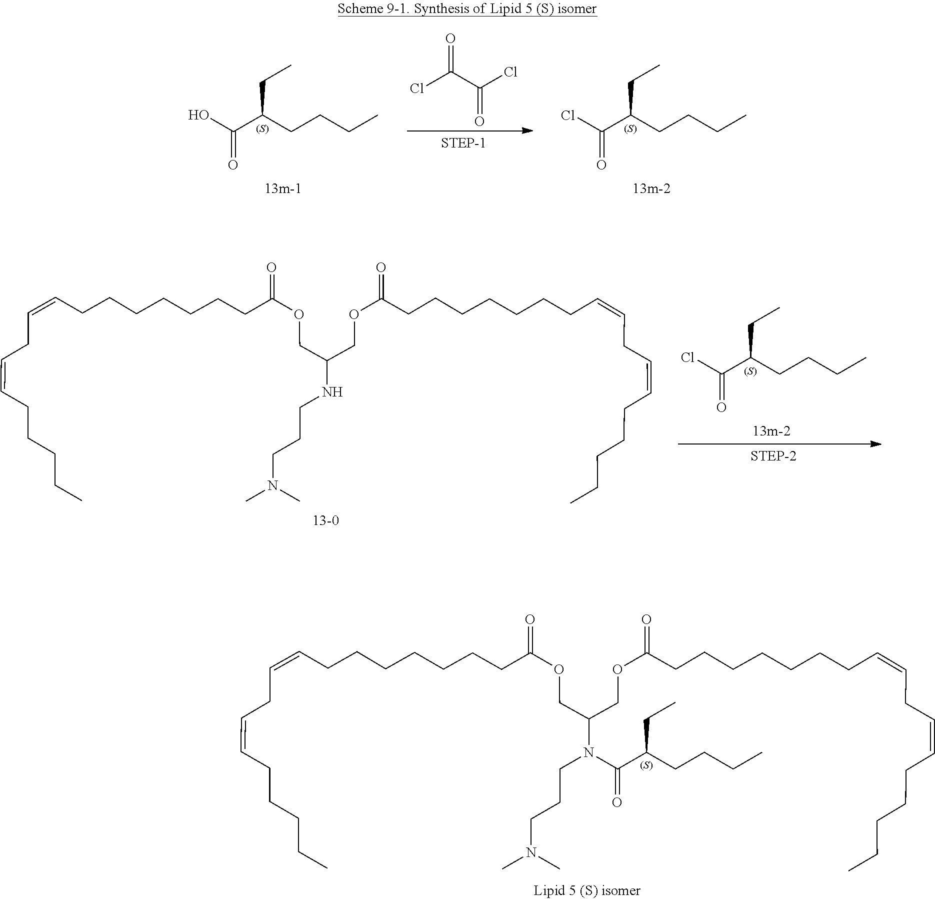

Lipid 5 as a racemic mixture was synthesized similarly as provided in scheme 9-2 below.

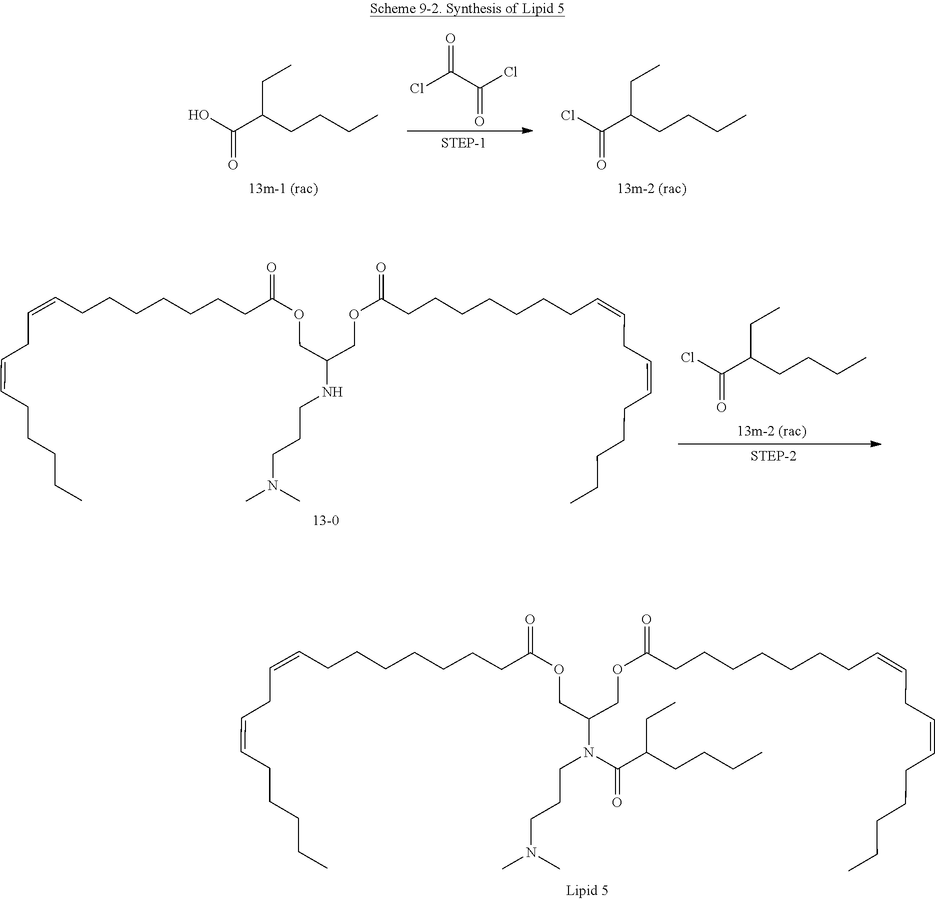

### Synthesis of Lipid 6

Lipid 6 was synthesized as provided in scheme 10 below and as follows. Starting material, 2-ethylnonanoic acid 13-14 (132 mg, 0.17 mmol, 1 eq.) was converted to the acid chloride, 13-14′ (Step 1) using oxalyl chloride (207 μl, 3.4 eq, 2.4 mmol) and DMF (10 μl, catalytic quantity) in 6 mL of benzene. Product showed only one spot on TLC (as methyl ester) and was used without further purification for acylation (step 2) of intermediate 13-0. Intermediate 13-0 (0.47 mmol, 330 mg, 1 eq.) was acylated with crude acid chloride, 13-14′ (145 mg, 1.5 eq, 0.7 mmol) using TEA (327 μL, 5.0 eq, 2.4 mmol) and DMAP (10 mg, catalytic amount) in 10 mL benzene. Crude product was purified by column chromatography (2×) yielding 75 mg (18%) of pure Lipid 6 (≥99% purity by LC-ELSD) and characterized by proton NMR and Mass Spectrometry (see FIG. 4E-1 for Lipid 6 NMR spectrum, FIG. 4E-2 for Lipid 6 LC-MS, and Table 5 for product mass).

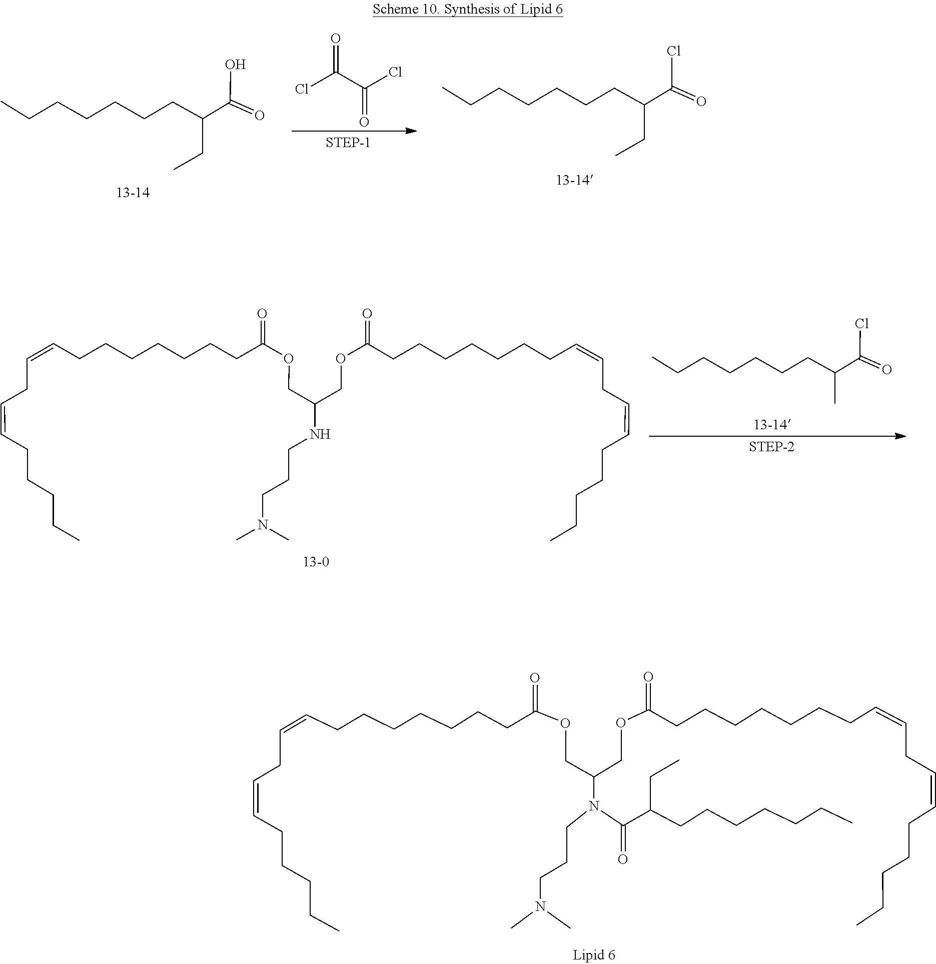 [SMILES: CCCCCCCC(CC)C(=O)O ; O=C(Cl)C(=O)Cl ; CCCCCCCC(CC)C(=O)Cl ; CCCCC/C=C\C/C=C\CCCCCCCC(=O)OCC(COC(=O)CCCCCCC/C=C\C/C=C\CCCCC)NCCCN(C)C ; CCCCCCCC(C)C(=O)Cl ; CCCCC/C=C\C/C=C\CCCCCCCC(=O)OCC(COC(=O)CCCCCCC/C=C\C/C=C\CCCCC)N(CCCN(C)C)C(=O)C(CC)CCCCCCC]

### Synthesis of Lipid 7

Lipid 7 was synthesized as provided in scheme 11 below and as follows. Starting material, heptanoic acid, 13-15 (23.1 mmol, 3.0 g, 1 eq.) was alkylated (step 1) with n-butyl bromide, 13-16 ((2.5 mL, 1.0 eq, 23.1 mmol) and 2.5 M n-butyl lithium in hexane (20.0 mL, 2.2 eq, 51 mmol) using diisopropylamine (7.2 mL, 2.2 eq, 51 mmol) in HMPA (4.4 mL) and 30 mL THF. 1.5 g (35%) of 2-butyl heptanoic acid, 13-17, was isolated from reaction mixture by flash chromatography. Intermediate 13-17 (360 mg, 0.94 mmol, 1 eq.) was converted to the acid chloride, 13-17′ (Step 2) using oxalyl chloride (6.6 mmol, 568 μl, 3.4 eq.) and DMF (5 μl, catalytic) in 3 mL of benzene. Product showed only one spot on TLC (as methyl ester) and was used without further purification for acylation (step 3) of intermediate 13-0. Intermediate 13-0 (0.64 mmol, 450 mg, 1 eq.) was acylated with crude acid chloride, 13-17′ (395 mg, 3.0 eq, 1.94 mmol), TEA (446 μL, 5.0 eq, 3.2 mmol), DMAP (10 mg) in 10 mL of benzene. Crude product was purified by column chromatography (2×) yielding 228 mg (41%) of pure Lipid 7 (≥99% purity by LC-ELSD) and characterized by proton NMR and Mass Spectrometry (see FIG. 4F-1 for Lipid 7 NMR spectrum, FIG. 4F-2 for Lipid 7 LC-MS, and Table 5 for product mass).

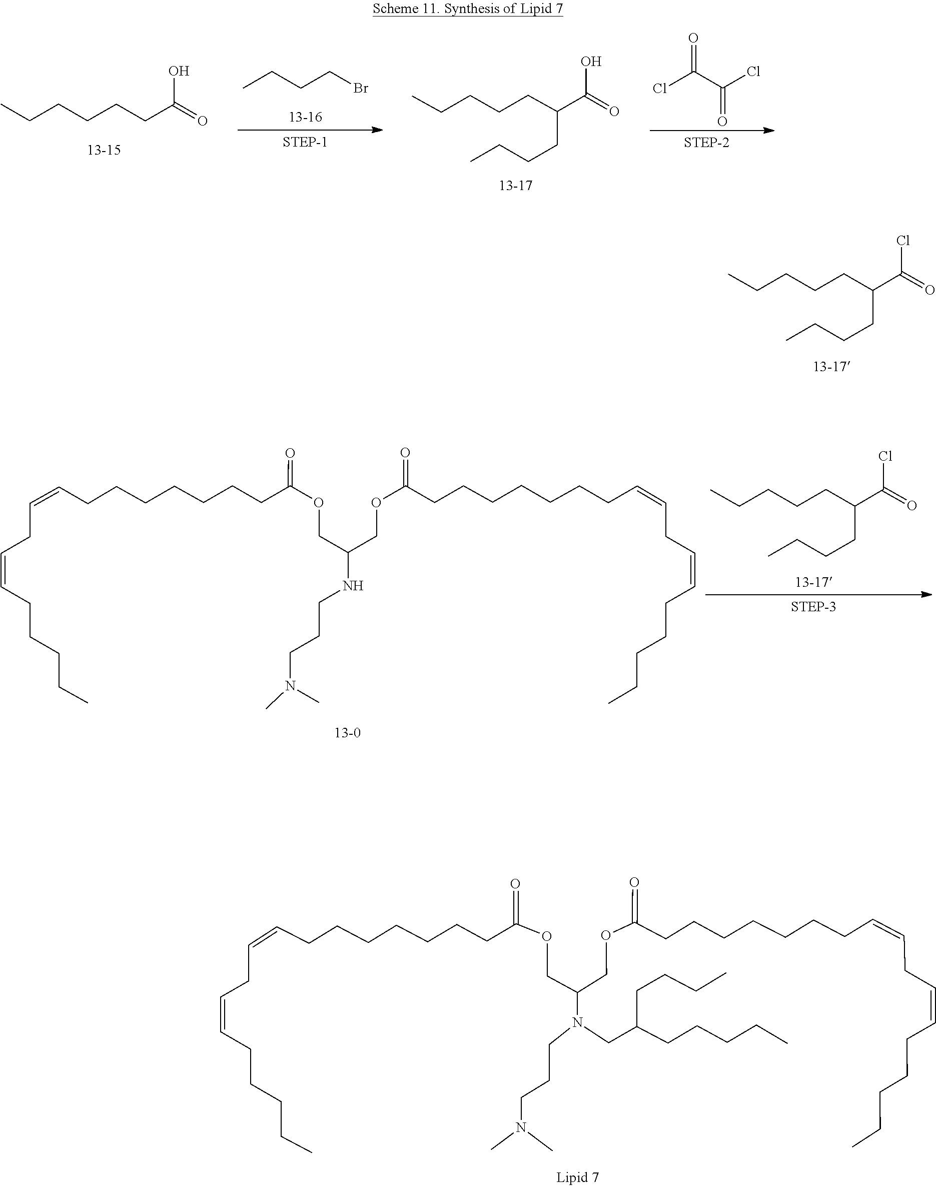

### Synthesis of Lipids 2, 8, 9 and 10 by N-Acylation of Intermediate 13-0 Using Carbodiimide Activation of the Corresponding Carboxylic Acids

### Synthesis of Lipid 2

Lipid 2 was synthesized as provided in scheme 12 below and as follows. Intermediate 13-0 (0.14 mmol, 320 mg, 1.0 eq.) was acylated with nonanoic acid 13-12 (1.15 mmol, 198 uL, 2.5 eq.), EDCI (1.15 mmol, 221 mg, 2.5 eq.), DIPEA (1.15 mmol, 198 uL, 2.5 eq.), and DMAP (0.05 mmol, 6.4 mg, 0.1 eq.) in 5 mL DCM. Crude product was purified by column chromatography (3×) yielding 107 mg (%) of pure Lipid 2 (≥99% purity by LC-ELSD) and characterized by proton NMR and Mass Spectrometry (see FIG. 4G-1 for Lipid 2 NMR spectrum, FIG. 4G-2 for Lipid 2 LC-MS, and Table 5 for product mass).

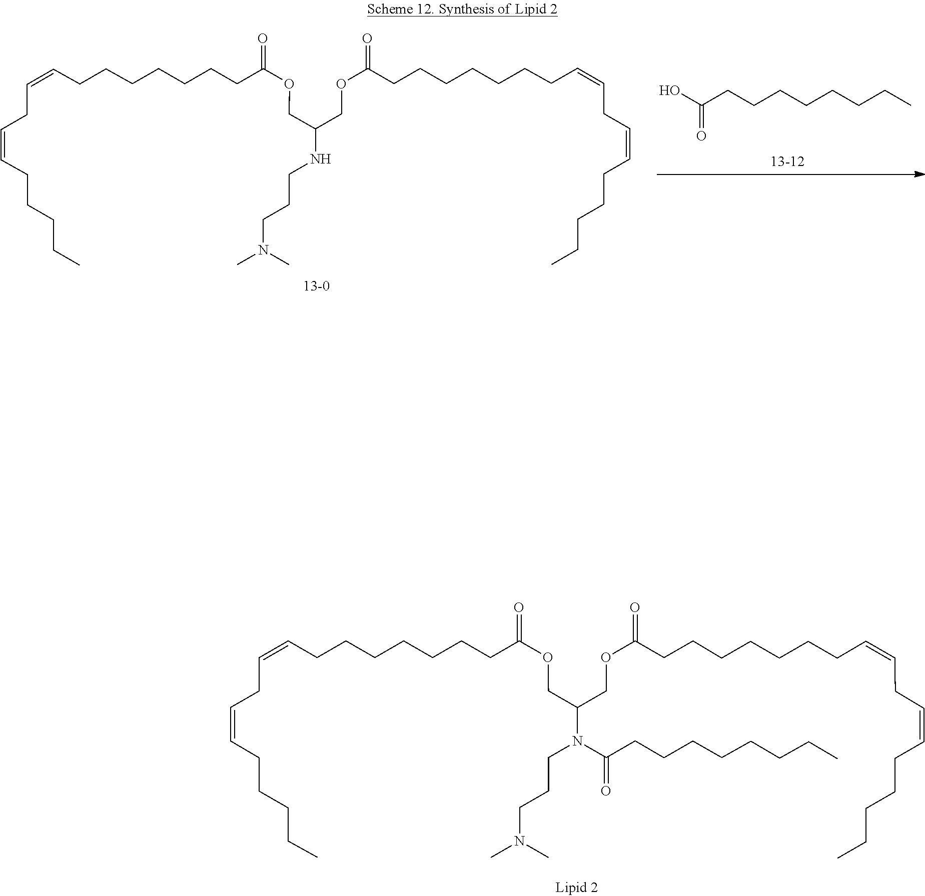 [SMILES: CCCCC/C=C\C/C=C\CCCCCCCC(=O)OCC(COC(=O)CCCCCCC/C=C\C/C=C\CCCCC)NCCCN(C)C ; CCCCCCCCC(=O)O ; CCCCC/C=C\C/C=C\CCCCCCCC(=O)OCC(COC(=O)CCCCCCC/C=C\C/C=C\CCCCC)N(CCCN(C)C)C(=O)CCCCCCCC]

### Synthesis of Lipid 8

Lipid 8 was synthesized as provided in scheme 13 below and as follows. Alkene, 13-48 was accessed via the HWE reaction (step 1) of octan-3-one, 13-46 (2 g, 15.6 mmol) with ethyl 2-(diethoxyphosphoryl)acetate, 13-47 (7.0 g, 2.0 eq, 31.2 mmol), 2M NaHMDS in THE (15.6 mL, 2.0 eq, 31.2 mmol), and 9 ml THE solvent. Reaction workup yielded 2.38 g (77%) of 13-48 confirmed by NMR, product mass and single TLC spot. Alkene, 13-48 (5.1 mmol, 1 g, 1 eq.) was hydrogenated (step 2) using Pd/C (50 mg) in 8 mL ethyl acetate yielding intermediate 13-48 (958 mg, 77%). Ester hydrolysis (step 3) of 13-49 (5.1 mmol, 412 mg) using THF/MeOH/1M LiOH (3.0/2.0/3.0 mL) yielded carboxylic acid intermediate 13-50 (336 mg, 95%). Intermediate 13-0 (0.33 mmol, 234 mg) was acylated with 13-50 (0.66 mmol, 115 mg, 2.0 eq.) using EDCI (0.66 mmol, 102 mg, 2.0 eq.), DIPEA (0.66 mmol, 114 μL, 2.0 eq.), DMAP (0.33 mmol, 41 mg, 1.0 eq.), in 2 mL DCM yielding 77 mg (27%) of pure Lipid 8 (≥99% purity by LC-ELSD) and characterized by proton NMR and Mass Spectrometry (see FIG. 4H-1 for Lipid 8 NMR spectrum, FIG. 4H-2 for Lipid 8 LC-MS, and Table 5 for product mass).

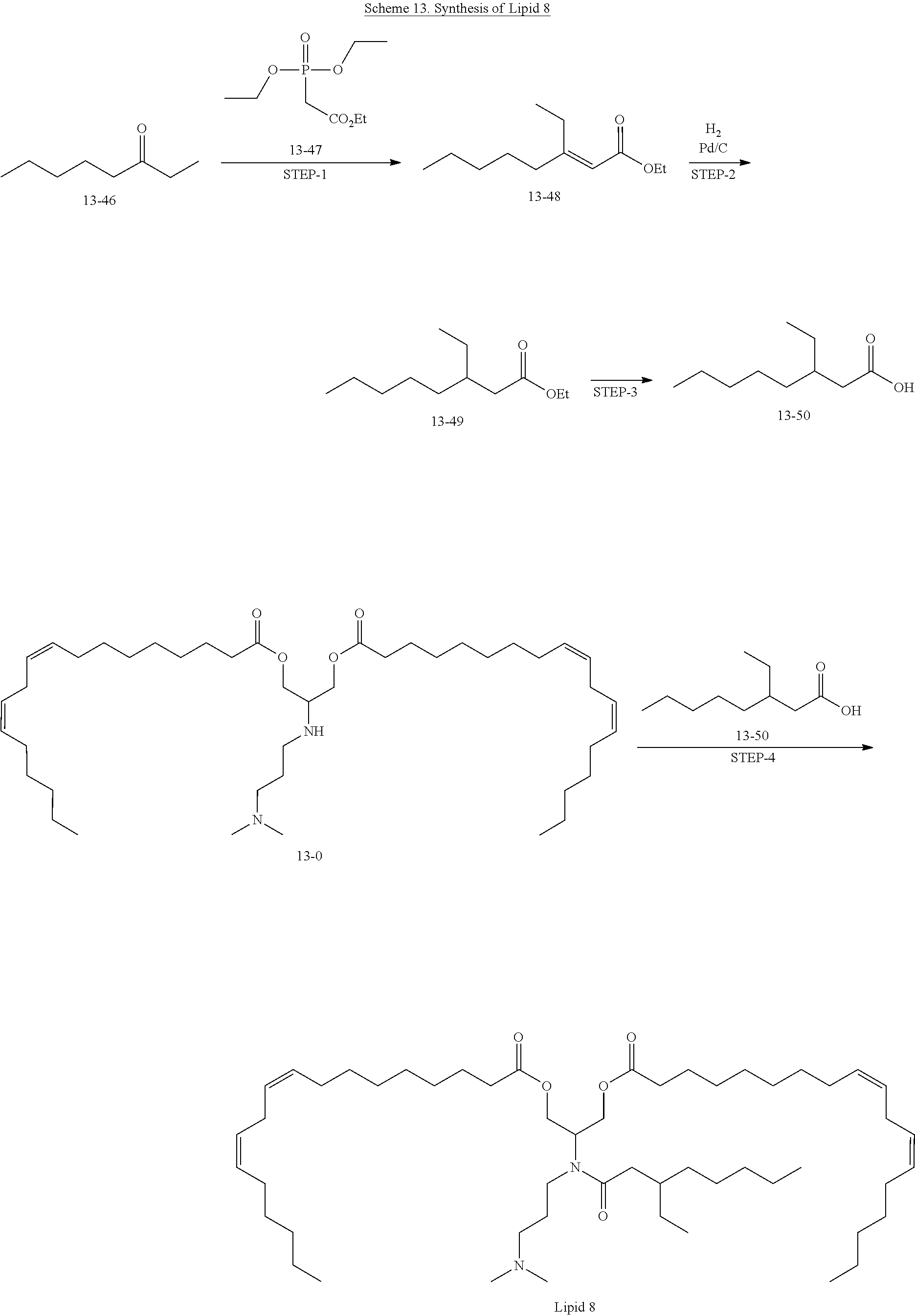

### Synthesis of Lipid 9

Lipid 9 was synthesized as provided in scheme 14 below and as follows. Starting material, decan-4-ol, 13-29 (32.0 mmol, 5.0 g, 1.0 eq.) was acylated with succinic acid, 13-30 (6.3 g, 2.0 eq, 63.0) using DMAP (3.55 g, 1.0 eq, 32.0 mmol) and pyridine (5.0 ml) in 5 mL THF. Crude product was purified by column chromatography (1×) to obtain 4.26 g (81%) of pure acid intermediate 13-31. Intermediate 13-0 (2.1 mmol, 1.5 g, 1 eq.) was acylated with 13-31 (2.13 mmol, 0.554 g, 1.1 eq), using DIPEA (745 μL, 4.26 mmol, 2.5 eq), EDCI (820 mg, 4.26 mmol, 2.5 eq), and DMAP (480 mg, 0.43 mmol, 0.25 eq), in 50 mL DCM. Crude product was purified by column chromatography (3×) yielding 1.4 g (73%) of pure Lipid 9 (≥99% purity by LC-ELSD) and characterized by proton NMR and Mass Spectrometry (see FIG. 4I-1 for Lipid 9 NMR spectrum, FIG. 4I-2 for Lipid 9 LC-MS, and Table 5 for product mass).

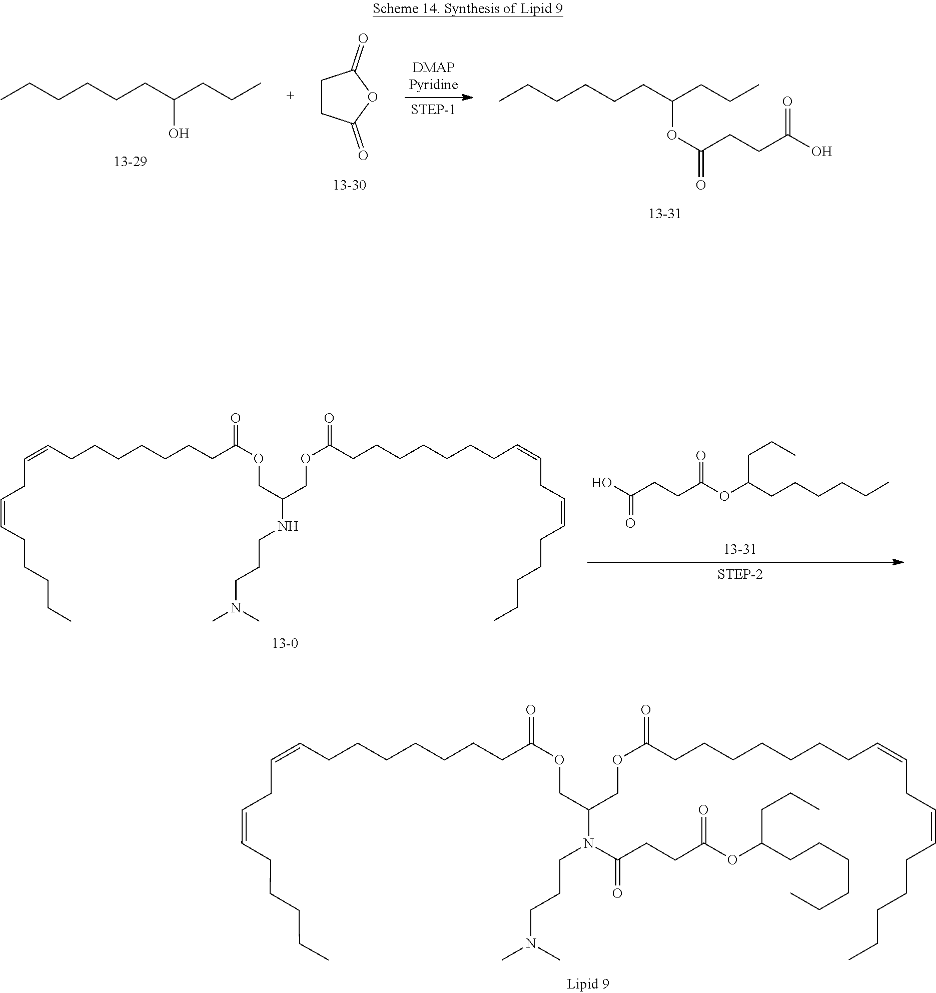

### Synthesis of Lipid 10 and its (S) Isomer

The (S) isomer of lipid 10 was synthesized as provided in scheme 15-1 below and as follows. Starting material, Octan-3-ol, 13-46 (2.0 g, 1.0 eq, 15.3 mmol) was acylated with succinic acid, 13-30 (3.1 g, 2.0 eq, 30.6 mmol) using DMAP (1.72 g, 1.0 eq, 15.3 mmol) and pyridine (2.0 ml) in 2 mL THE and 6 mL DCM. Crude product was purified by column chromatography (1×) to obtain 1.1 g (31%) of pure acid intermediate 13-47. Intermediate 13-0 (250 mg, 1.0 eq, 0.36 mmol) was acylated with 13-47 (123 mg, 1.5 eq, 0.53 mmol), using EDCI (207 mg, 3.0 eq, 1.80 mmol), DIPEA (188 μL, 3.0 eq, 1.8 mmol) and DMAP (15.0 mg, 3.0 eq, 0.018 mmol), in 5 mL DCM. Crude product was purified by column chromatography (2×) yielding 261 mg (54%) of pure Lipid 10 (≥99% purity by LC-ELSD) and characterized by proton NMR and Mass Spectrometry (see FIG. 4J-1 for Lipid 10 NMR spectrum, FIG. 4J-2 for Lipid 10 LC-MS, and Table 5 for product mass).

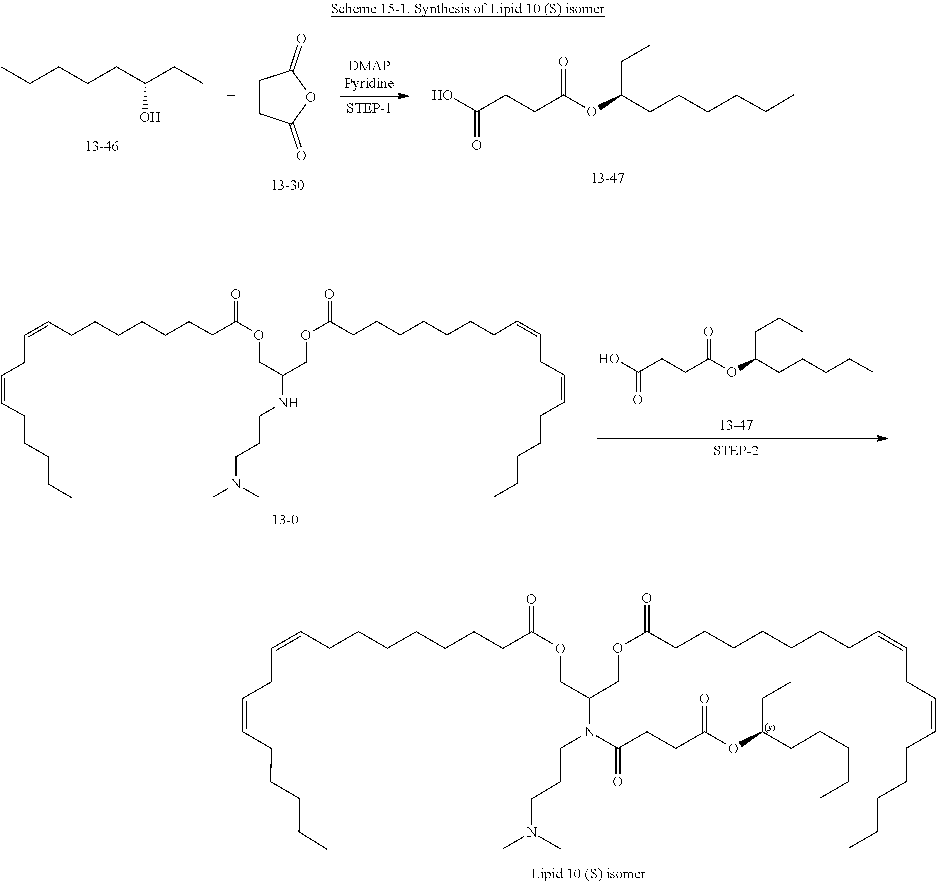

Lipid 10 as a racemic mixture was synthesized similarly as provided in scheme 15-2 below. Starting material, octan-3-ol, 13-46 (2.0 g, 1.0 eq, 15.3 mmol) was acylated with succinic acid, 13-30 (3.1 g, 2.0 eq, 30.6 mmol) using DMAP (1.72 g, 1.0 eq, 15.3 mmol) and pyridine (2.0 ml) in 2 mL THE and 6 mL DCM to obtain intermediate 13-47. Crude product was purified by column chromatography (1×) to obtain 1.1 g (31%) of pure acid intermediate 13-47. 13-0 (250 mg, 1.0 eq, 0.36 mmol) was acylated with 13-38 (123 mg, 1.5 eq, 0.53 mmol) using DIPEA (188 μL, 3.0 eq, 1.8 mmol), EDCI (207 mg, 3.0 eq, 1.80 mmol), and DMAP (15.0 mg, 3.0 eq, 0.018 mmol), in 5 mL DCM. Crude product was purified by column chromatography (2×) yielding 261 mg (54%) of pure Lipid 10 (≥99% purity by LC-ELSD) and characterized by proton NMR and Mass Spectrometry (see FIG. 4J-1 for Lipid 10 NMR spectrum, FIG. 4J-2 for Lipid 10 LC-MS, and Table 5 for product mass).

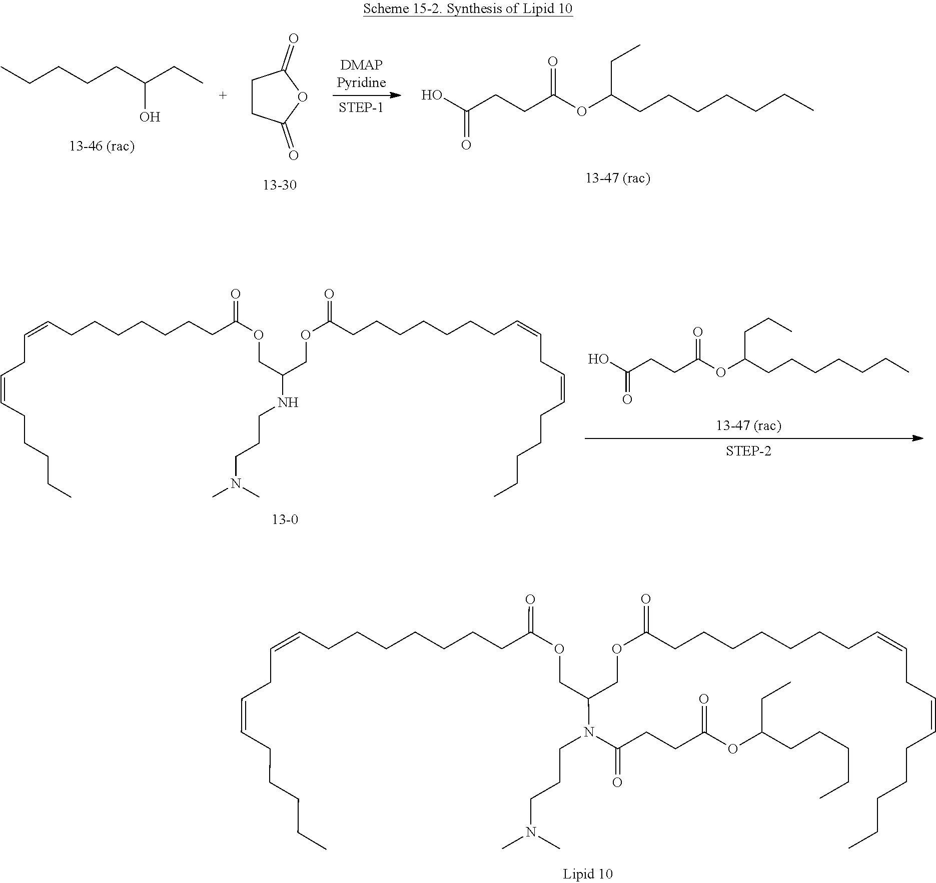

### Synthesis of Lipid 11 by N-Acylation of Intermediate 13-0 Using the Corresponding Acid Chloride

### Synthesis of Lipid 11 and its (S) Isomer

The (S) isomer of lipid 5 was synthesized as provided in scheme 16-1 below and as follows. Starting material, benzyl alcohol, 13-39′ (18.5 mmol, 2 g) was used to acylate compound 13-39 (4.8 g, 1.5 eq, 27.8 mmol) using EDCI (5.4 g, 1.5 eq, 27.8 mmol), DIPEA (4.6 mL, 1.5 eq, 27.8 mmol), and DMAP (463 mg, 0.2 eq, 3.7 mmol) yielding 3.6 g (74%) of column purified intermediate 13-40 (product confirmed by mass spectrometry and proton NMR). Intermediate, 13-40 (684 mg, 2.6 mmol, 1 eq.) was deprotected in acetic acid to obtain intermediate, 13-41 (˜600 mg, quantitative and product structure was confirmed by mass spectrometry and proton NMR). Additional quantity of intermediate 13-41 was generated and 1.68 g, 7.5 mmol of 13-41 was selectively protected at the hydroxyl group using TBSCl (1.7 g, 11.25 mmol, 1.5 eq), TEA (5.3 mL, 5.0 eq, 37.5 mmol), and DMAP (92 mg, 0.75 mmol, 0.1 eq), in 20 mL DCM yielding protected intermediate 13-41a (˜2.5 g, quantitative) (product mass was confirmed by mass spectrometry and proton NMR). Intermediate 13-41a (1.61 g, 4.76 mmol) was esterified with n-hexyl alcohol 13-34 (2.94 mL, 23.8 mmol, 5.0 eq) using EDCI (2.76 g, 14.2 mmol, 3.0 eq), DIPEA (1.6 mL, 2.0 eq, 9.52 mmol), and DMAP (580 mg, 4.76 mmol, 1.0 eq) in 11.0 mL of DCM to obtain 13-41b (0.95 g, 48%). Additional quantity of 13-41b was generated and total of 1.36 g (3.2 mmol) was deprotected using HF-pyridine (5.8 mL, 80.6 mmol, 25 eq.) in 30 mL THE to obtain intermediate 13-41c (837 mg, 84%). Intermediate 13-41c (456 mg, 1.48 mmol) was acylated with n-butanoyl chloride, 13-42 (760 μL, 7.4 mmol, 5.0 eq) in 4.0 mL pyridine (4.0 mL) yielding compound 13-44 (505 mg, 90%). Intermediate 13-44 (505 mg, 1.34 mmol) was deprotected using Pd/C (30 mg) in 3.0 mL ethyl acetate yielding compound 13-45 (370 mg, 96%). Compound 13-45 (188 mg, 0.65 mmol) was converted to the acid chloride intermediate using oxalyl chloride (190 μg, 3.4 eq, 2.2 mmol) and DMF (10 μL, catalytic quantity), in 3 mL Benzene. Product showed only one spot on TLC (as methyl ester) and was used without further purification for acylation (step 9) of intermediate 13-0. Intermediate 13-0 (152 mg, 0.22 mmol, 1 eq.) was acylated with crude acid chloride, 13-45′ (200 mg, 3.0 eq, 0.65 mmol), TEA (152 μL, 5.0 eq, 1.1 mmol), DMAP (10 mg) in 5 mL of benzene to obtain Lipid 11. Crude product was purified by column chromatography to yield 77 mg (37%) of pure Lipid 11 (≥99% purity by LC-ELSD) and characterized by proton NMR and Mass Spectrometry (see FIG. 4K-1 for Lipid 11 NMR spectrum, FIG. 4K-2 for Lipid 11 LC-MS, and Table 5 for product mass).

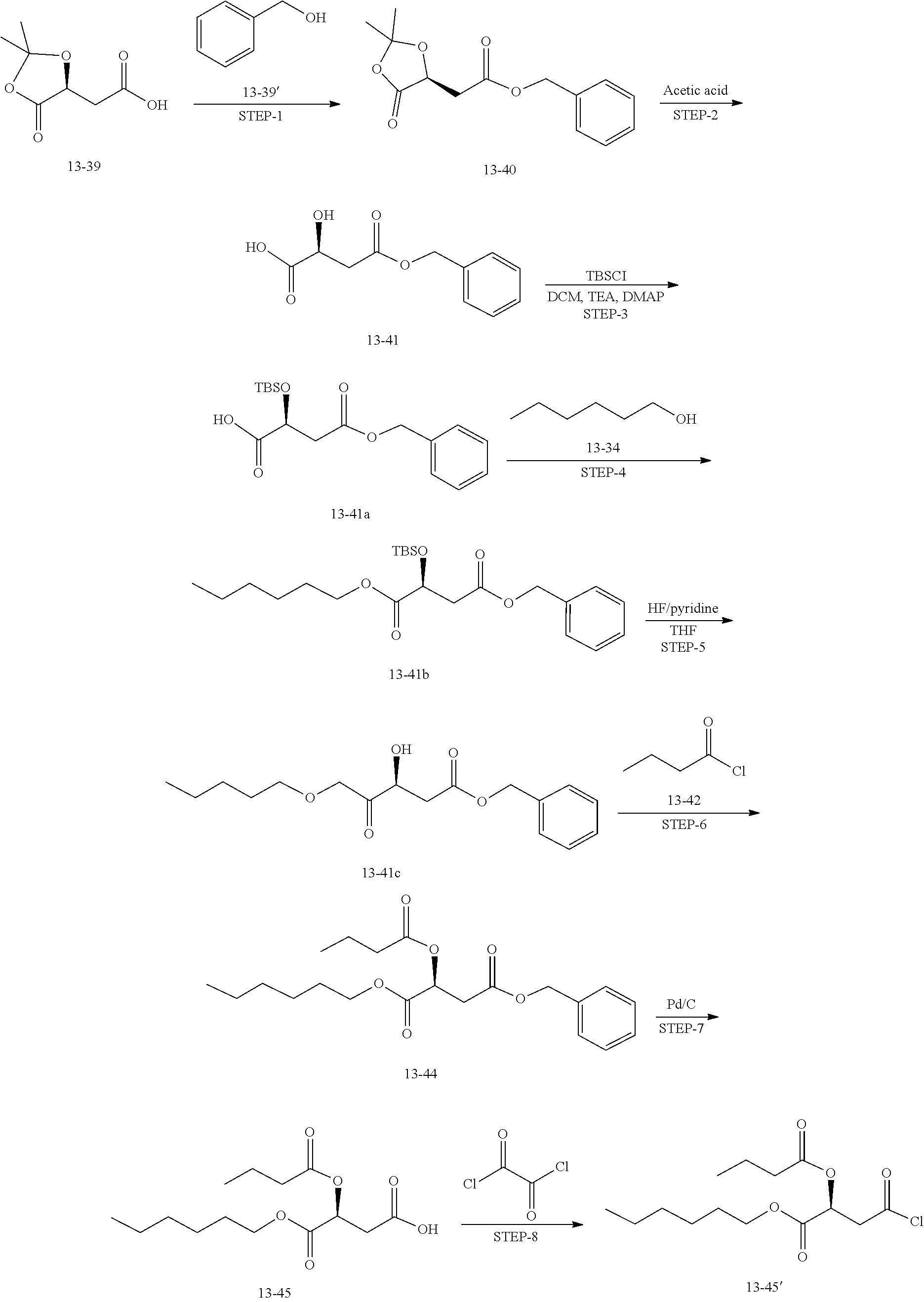

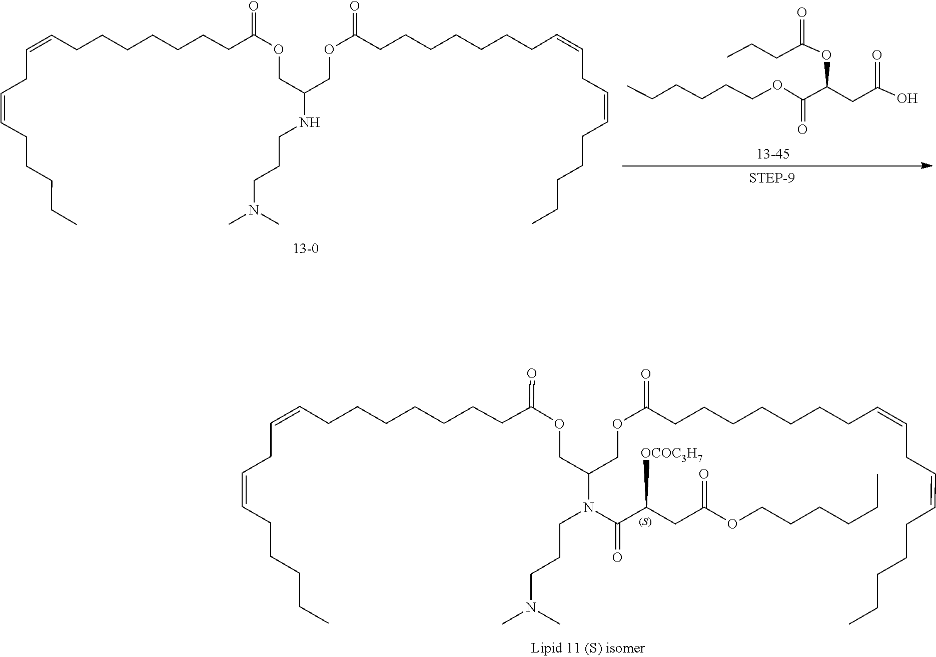

Lipid 11 as a racemic mixture was synthesized similarly as provided in scheme 16-2 below.

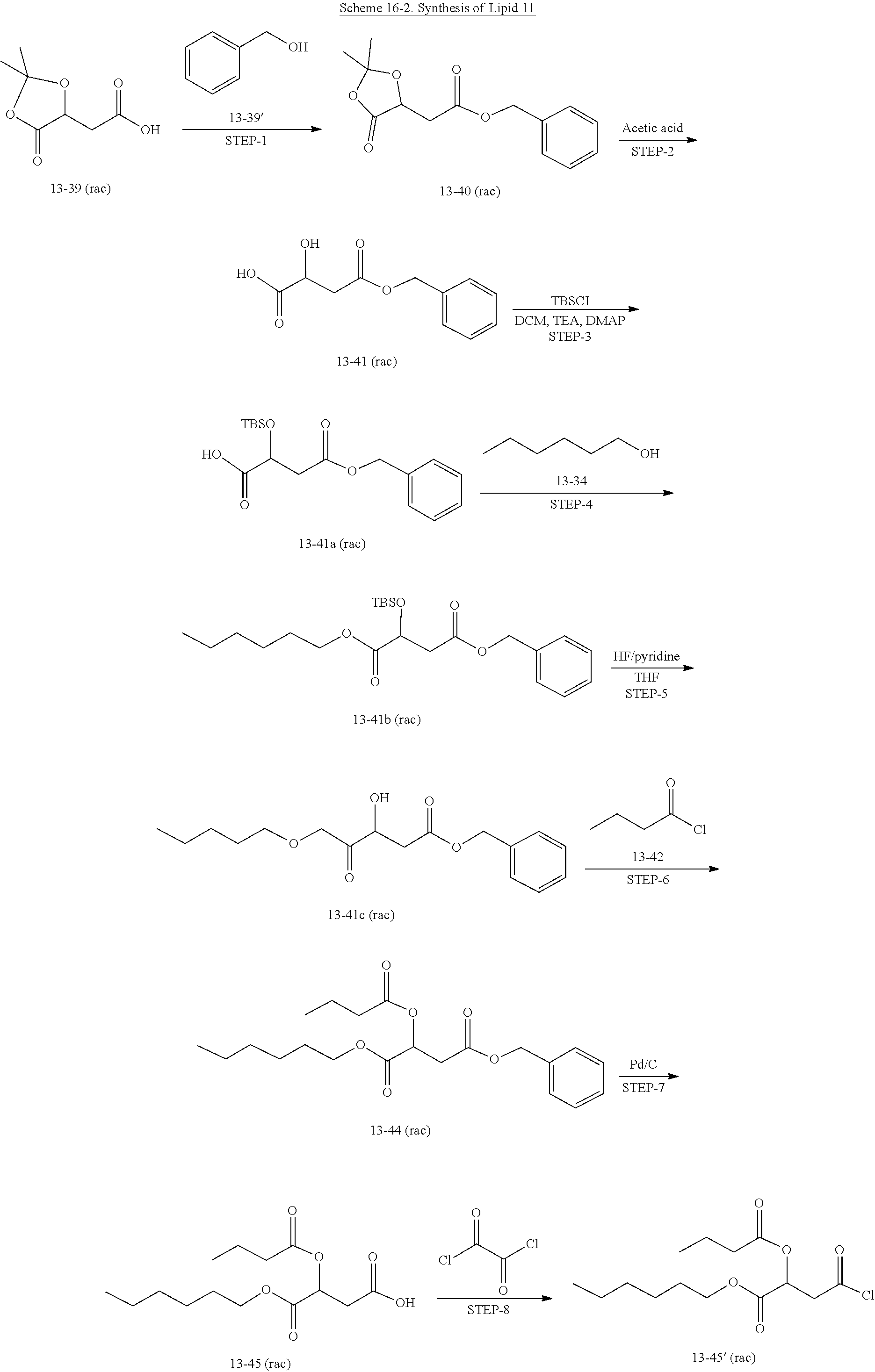

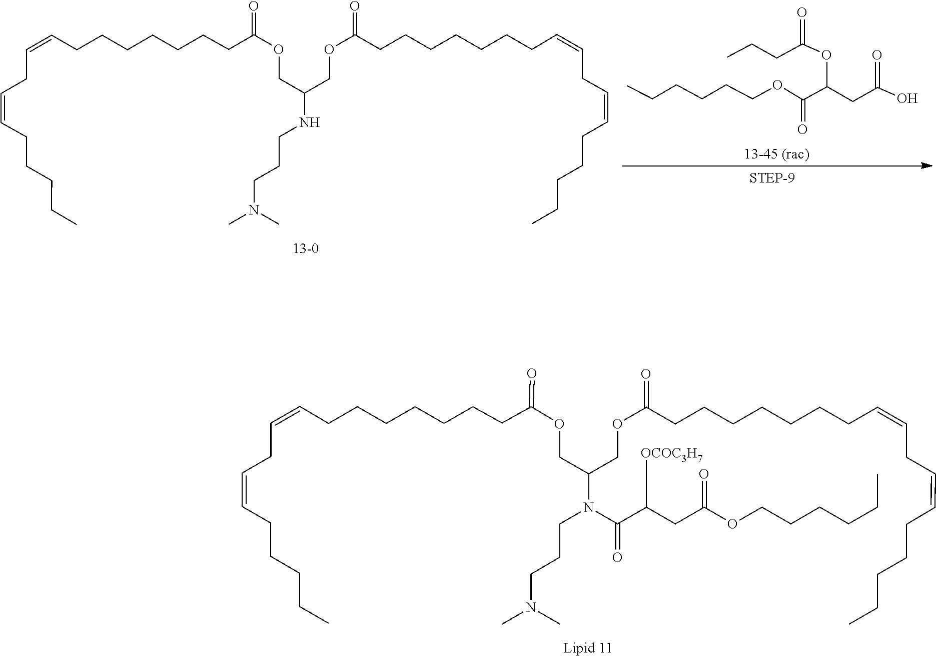

### Synthesis of Lipid 12

Lipid 12 was synthesized as provided in scheme 34 below and as follows. Starting material, 14-3 (3 g, 1.0 eq, 22.37 mmol) was selectively protected in trifluoroacetic anhydride (11.27 g, 2.4 eq, 53.69 mmol) and Benzyl alcohol (15 mL) at room temperature, overnight yielding intermediate 14-4. Crude product was purified by column chromatography (1×) to obtain 4.7 g (96%) purified 14-4. Subsequent acylation of 14-4 (1.0 eq, 4.44 mmol) with n-butanol, 13-34 (4.55 g, 10.0 eq, 44.60 mmol) using EDCI (1.71 g, 2 eq, 8.92 mmol) and DMAP (1.089 g, 2 eq, 8.92 mmol) in 10 mL DCM at RT, overnight yielded 14-5. Crude product was purified by column chromatography (1×) to obtain 800 mg (58%) purified 14-5. Acylation of the free hydroxyl of 14-5 (800 mg, 1.0 eq, 2.59 mmol) with hexanoyl chloride (1.39 g, 4.0 eq, 10.37 mmol) using TEA (1.31 g, 5 eq, 12.97 mmol) and DMAP (10 mg, catalytic amount) in 10 mL toluene at room temperature, overnight yielded intermediate 14-7. Purification of crude product by column chromatography (1×) yielded 470 mg (46%) purified 14-7. Intermediate 14-7 (470 mg, 1 eq., 3.4 mmol) yielded 340 mg (93%) of free acid 14-8. Crude 14-8 (56 mg. 1 eq., 0.18 mmol) was converted to the corresponding chloride, 14-8′, using Oxalyl Chloride (50 μL, 3.4 eq, 0.60 mmol) and DMF (0.2 μL, Catalytic amount) in 1 mL Toluene at room temperature, overnight to afford 56 mg of crude chloride 14-8′. N-acylation of 13-0 (42 mg, 1 eq., 0.059 mmol) with 14-8′ (56.0 mg, 3.0 eq, 0.17 mmol) using TEA (39.0 μL, 5.0 eq, 0.29 mmol) and DMAP (10 mg, Catalytic amount) in 3 mL Toluene yielded Lipid 12. Crude product was purified by column chromatography (1×) to obtain pure Lipid 12 (23 mg, 39%) (≥99% purity by LC-ELSD) and characterized by proton NMR and Mass Spectrometry (see FIG. 4L-1 for Lipid 12 NMR spectrum, FIG. 4L-2 for Lipid 12 LC-ELSD chromatogram, and Table 5 for product mass).

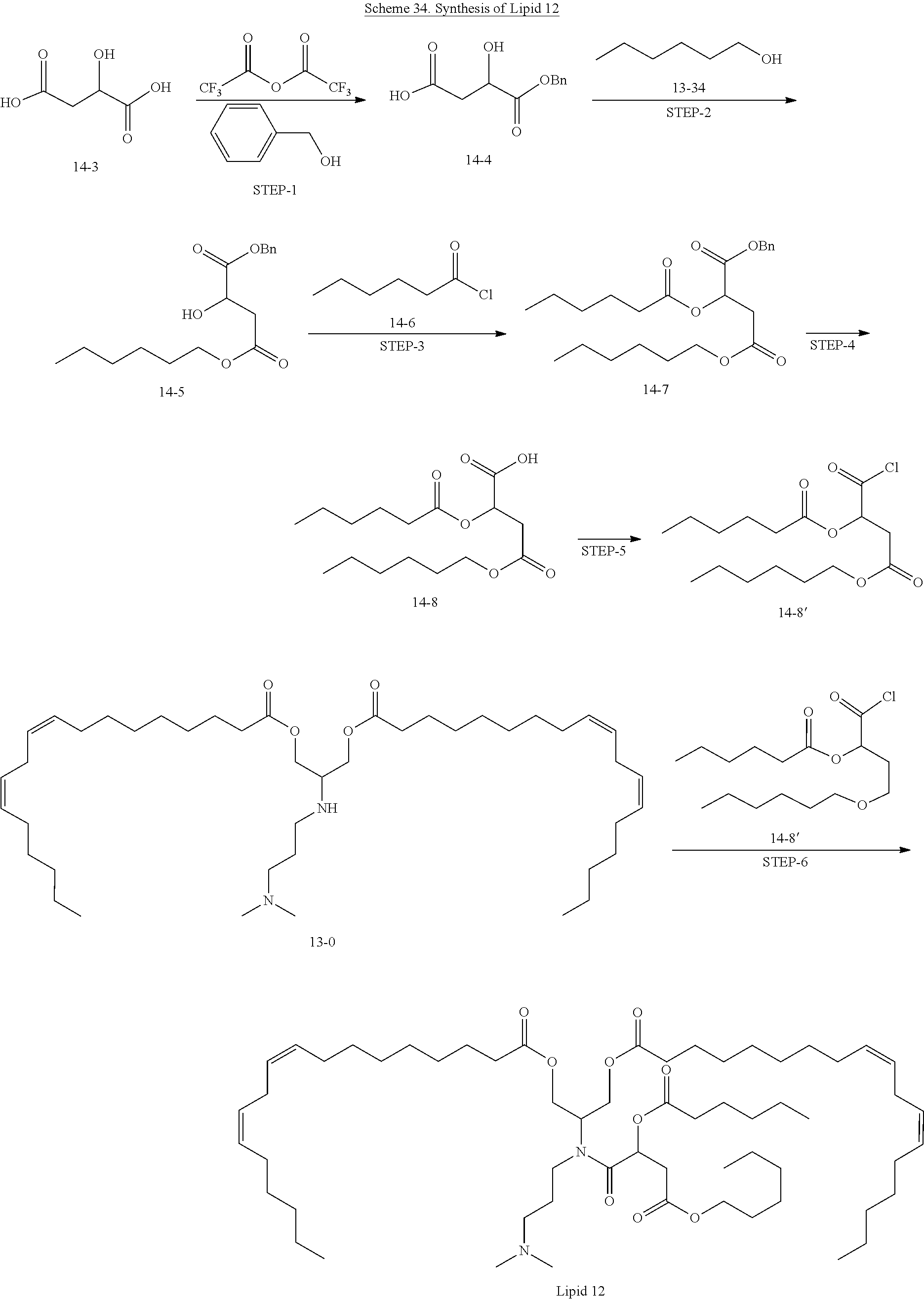

### Synthesis of Lipids 13 by N-Acylation of Intermediate 13-0 Carbodiimide Activation of the Corresponding Carboxylic Acid

### Synthesis of Lipid 13

Lipid 13 was synthesized as provided in scheme 17 below and as follows. Starting material 13-32 (4.8 g, 2.0 eq, 25.0 mmol) was esterified with 1-Butanol (1.13 mL, 1 eq, 12.4 mmol) using EDCI (4.8 g, 2 eq, 25.0 mmol), DIPEA (4.35 mL, 2 eq, 25.0 mmol), and DMAP (280 mg, 0.2 eq, 2.5 mmol) in 20 mL DCM to obtain intermediate 13-33. Crude product was purified by column chromatography to obtain 2.78 g (44%) of pure intermediate 13-33. Intermediate 13-36 was accessed by acylation of n-hexanol (2 g, 1.0 eq, 19.6 mmol) with 2-bromoacetyl bromide, 13-35 (5.05 g, 1.3 eq, 25.0 mmol) using NaHCO₃ (3.95 g, 2.4 eq, 47.0 mmol) in 50 mL acetonitrile. Crude product was purified by column chromatography (1×) to obtain 4.32 g (97%) of pure intermediate 13-36.

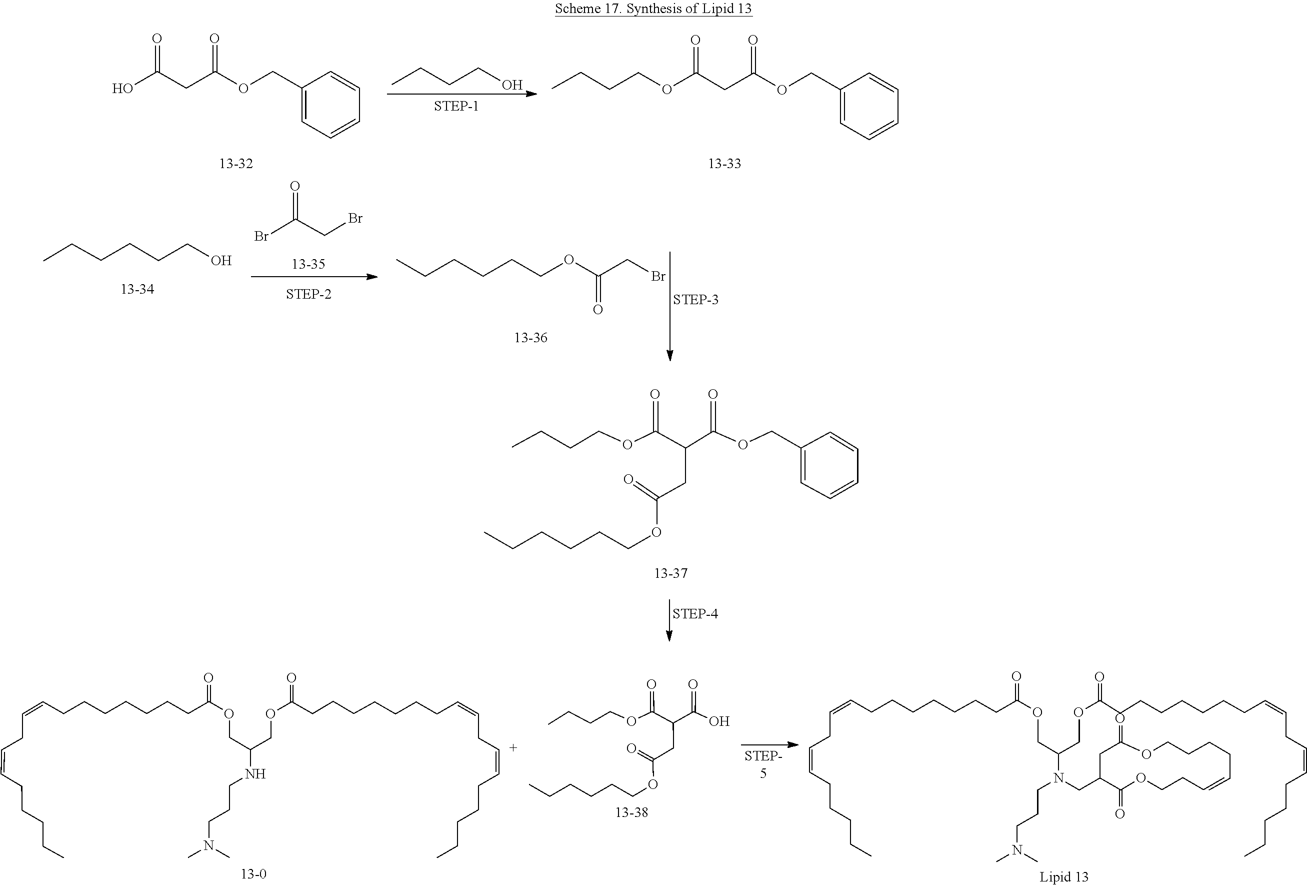

Intermediate 13-37 was accessed by in situ generation of the nucleophilic carbanion of 13-33 (1.25 g, 1.0 eq., 5.0 mmol) using NaH (200 mg, 1.0 eq, 5.0 mmol) in 8 mL DMF and displacement reaction with intermediate 13-36 (1.1 g, 1.0 eq, 5.0 mmol). Crude product was purified by column chromatography (2×) to 1.15 g (58%) of pure intermediate 13-37. Free acid intermediate 13-38 was obtained by deprotection (Pd/C, 230 mg catalyst and hydrogen gas in methanol) of intermediate 13-37 (1.15 g, 1.0 eq, 2.9 mmol). Crude product was purified by column chromatography (4×) to obtain 88 mg (9%) of pure intermediate 13-38. Intermediate 13-0 (105 mg, 1.0 eq, 0.04 mmol) was acylated with 13-38 (2.13 mmol, 0.554 g, 1.1 eq), using DIPEA (78 μL, 3.0 eq, 0.45 mmol), EDCI (87 mg, 3.0 eq, 0.45 mmol), and DMAP (5 mg, 0.3 eq, 0.04 mmol), in 2 mL DCM. Crude product was purified by column chromatography (3×) yielding 41 mg (27%) of pure Lipid 13 (≥99% purity by LC-ELSD) and characterized by proton NMR and Mass Spectrometry (see FIG. 4M-1 for Lipid 13 NMR spectrum, FIG. 4M-2 for Lipid 13 LC-MS, and Table 5 for product mass).

### Synthesis of Lipid 14A by N-Acylation of Intermediate 14-11 Using the Corresponding Acid Chloride

### Synthesis of Lipid 14A

Lipid 14A was synthesized as provided in Scheme 37 below and as follows.

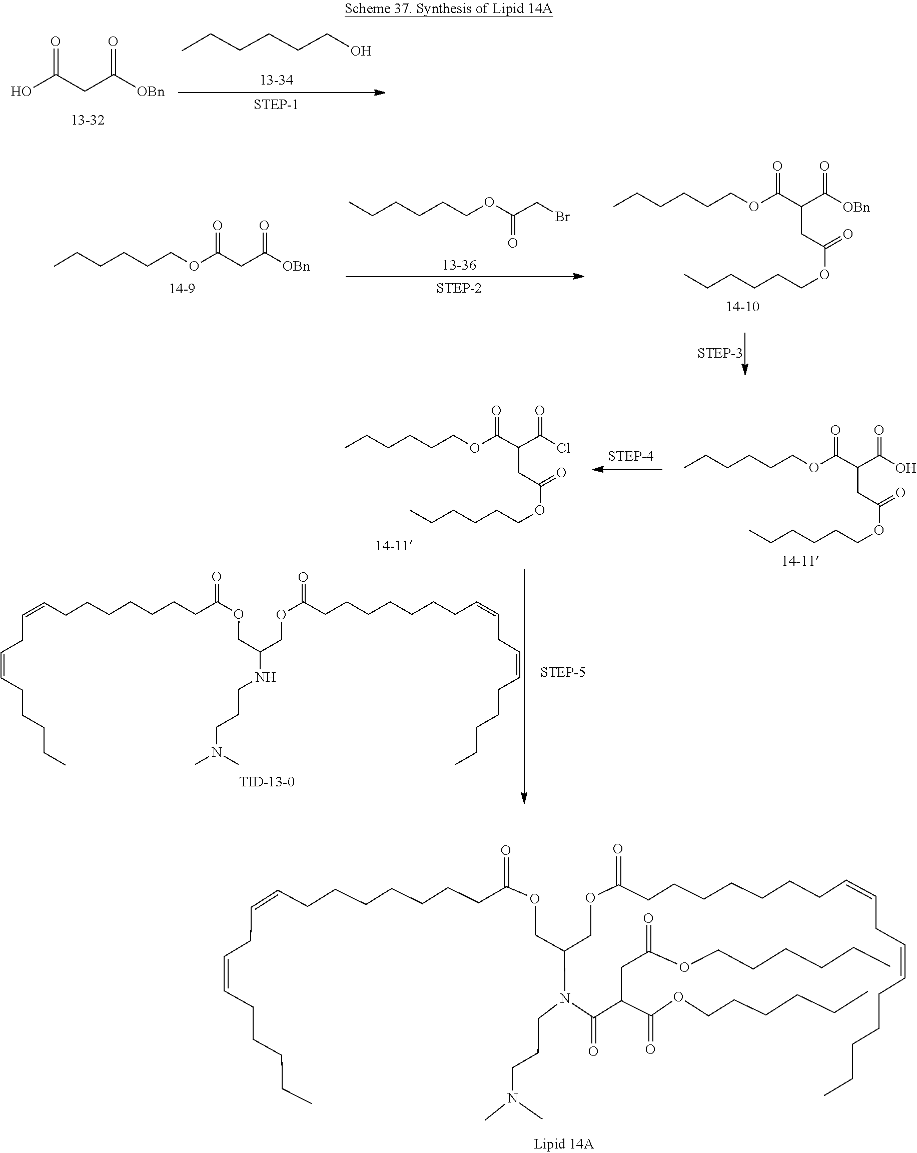

Starting material, mono-benzyl protected malonic acid, 13-32 (9.26 mmol, 1.8 g, 1.0 eq.) was esterified with N-hexanol, 13-34 (92.69 mmol, 9.47 g, 10.0 eq.) using EDCI (18.53 mmol, 3.55 g, 2 eq.) and DMAP (2.26 g, 2 eq, 18.53 mmol) in DCM (20 mL) at room temperature, overnight to obtain intermediate, 14-9 (2.04 g, 79%), Compound 13-36 was prepared by reacting bromoacetyl bromide (38.17 mmol, 7.70 g, 1.3 eq.) with 3.0 g of N-hexanol, 13-34 (1.0 eq, 29.36 mmol) using NaHCO₃ (5.9 g, 2.4 eq, 70.47 mmol) in 30 mL of acetonitrile (0° C. to room temperature) overnight to obtain 6.0 g (91.6%) of intermediate 13-36. Intermediate 14-9 (7.2 mmol, 2.03 g, 1.0 eq.) was converted to the corresponding carbanion and reacted with Bromoacetyl bromide (7.70 g, 1.3 eq, 38.17 mmol) using NaHCO₃ (5.9 g, 2.4 eq, 70.47 mmol) in 30 mL of acetonitrile (0° C. to room temperature, overnight) to obtain Intermediate, 14-10 (1.15 g, 38%).

Deprotection of Intermediate 14-10 (3.4 mmol, 1.15 g, 1.0 eq.) by hydrogenation (Pd/C, H₂, RT, overnight) in ethyl acetate yielded Intermediate 14-11 (850 mg, 94%). 14-11 (300 mg, 0.9 mmol, 1.0 eq.) was converted to the corresponding chloride, 14-11′ using Oxalyl Chloride (3.0 mmol, 260 μL, 3.4 eq,) in 4 mL Toluene using DMF (0.2 μL, Catalytic quantity) at room temperature for 2 hours. Crude 14-11′ (0.17 mmol, 280 mg, 3.0 eq.) was used for N-acylation of Intermediate 13-0 (0.059 mmol, 200 mg, 1.0 eq.) using TEA (39.0 μL, 5.0 eq, 0.29 mmol), DMAP (10 mg, Catalytic quantity) in 3 mL of Toluene to obtain Lipid 14A. Purification of crude product by column chromatography (DCM:10% MeOH in DCM) yielded 220 mg (76%) of purified Lipid 14A (98% by HPLC-CAD) and characterized by proton NMR and mass spectrometry (FIG. 4V-1 and FIG. 4V-2 for proton NMR and HPLC-CAD and Table 5 for Mass Spectrometry data).

### Synthesis of Lipid 15 by N-Acylation of Intermediate 13-11a Using the Corresponding Acid Chloride

### Synthesis of Lipid 15

Lipid 15 was synthesized as provided in scheme 18 below and as follows. Starting material, decan-4-ol, 13-29 (10.0 g, 63.0 mmol) was acylated with succinic acid, 13-30 (12.6 g, 126 mmol, 2.0 eq) using DMAP (7.7 g, 63 mmol, 1 eq) and pyridine (5.0 ml) in 5 mL THE and 15 mL DCM to obtain intermediate 13-31. Crude product was purified by column chromatography (3×) to obtain 8.9 g (55%) of pure acid intermediate 13-31. Intermediate 13-31 (1.26 g, 4.9 mmol) was converted to the acid chloride intermediate 13-31′ using oxalyl chloride (1.43 mL, 3.4 eq, 16.66 mmol) and DMF (50 μL, catalytic quantity), in 5 mL Benzene. Product showed only one spot on TLC (as methyl ester) and was used without further purification for acylation (step 3) of intermediate 13-11b. Intermediate 13-11b (275 mg, 0.39 mmol) was acylated with crude acid chloride, 13-31′ (324 mg, 3.0 eq, 1.17 mmol), TEA (270 μL, 5.0 eq, 1.95 mmol), DMAP (20 mg, catalytic quantity) in 10 mL of benzene to obtain Lipid 15. Crude product was purified by column chromatography (2×) to yield 230 mg g (64%) of pure Lipid 15 (99% purity by LC-ELSD) and characterized by proton NMR and Mass Spectrometry (see FIG. 4N-1 for Lipid 15 NMR spectrum, FIG. 4N-2 for Lipid 15 LC-MS, and Table 5 for product mass).

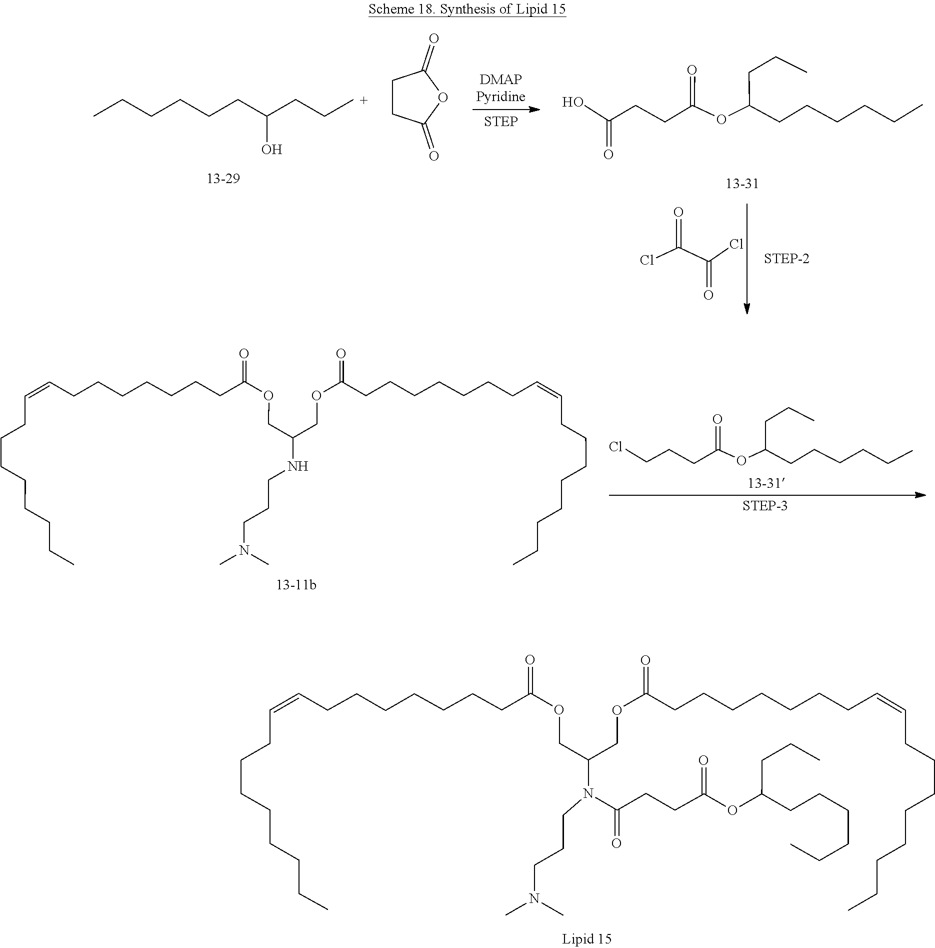

### Synthesis of Lipid 16

Lipid 16 was synthesized as provided in scheme 19 below and as follows. Starting material, octan-3-ol, 13-48 rac (3 g, 23 mmol) was acylated with succinic acid, 13-30 (46.08 mmol, 4.61 g, 2.0 eq) using DMAP (23.04 mmol, 2.8 g, 1.0 eq) and pyridine (5.0 ml) in 5 mL THE and 15 mL DCM to obtain intermediate 13-31. Crude product was purified by column chromatography (1×) to obtain 3.4 g (64%) of pure acid intermediate 13-47 rac. Intermediate 13-47 rac (300 mg, 0.42 mmol) was converted to the acid chloride intermediate 13-47′ rac using oxalyl chloride (0.38 mL, 4.4 mmol, 3.4 eq.) and DMF (2 μL, catalytic quantity). Product showed only one spot on TLC (as methyl ester) and was used without further purification for acylation (step 3) of intermediate 13-11b. Intermediate 13-11b (270 mg, 0.38 mmol) was acylated with crude acid chloride, 13-47′ rac (0.42 mmol, 300 mg, 3.0 eq.), TEA (260 μL, 5.0 eq, 1.9 mmol), DMAP (20 mg, catalytic quantity) in 5 mL of toluene to obtain Lipid 16. Crude product was purified by column chromatography (1×) to yield 165 mg (47%) of pure Lipid 16 (99% purity by LC-ELSD) and characterized by proton NMR and Mass Spectrometry (see FIG. 4O-1 for Lipid 16 NMR spectrum, FIG. 4O-2 for Lipid 16 LC-MS, and Table 5 for product mass).

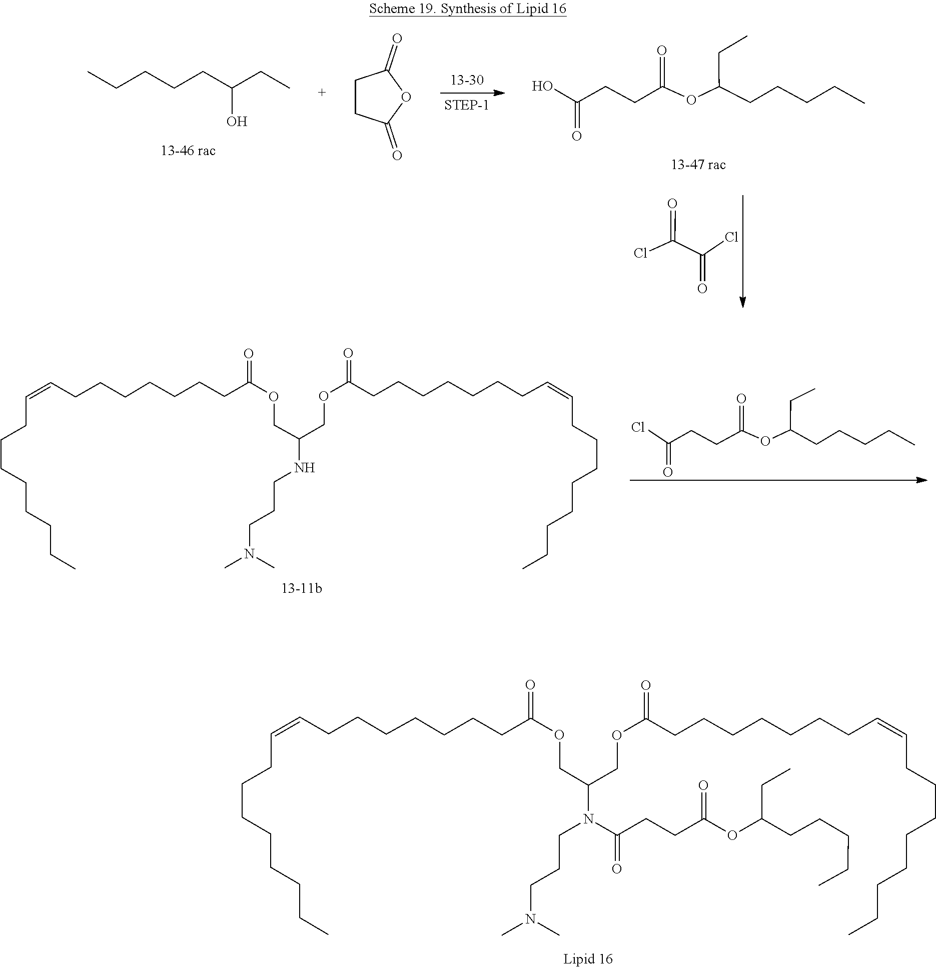

### Synthesis of Lipid 17

Lipid 17 was synthesized as provided in scheme 20 below. Octanedioic acid, 13-51 (5.0 g, 2.0 eq, 28.5 mmol) was mono-acylated with decane-3-ol, 13-29 (2.75 mL, 1.0 eq, 14.3 mmol) using EDCI (3.29 g, 1.2 eq, 17.2 mmol), DMAP (160 mg, 0.12 eq, 1.72 mmol) and TEA (9.96 mL, 5.0 eq, 71.5 mmol) in 50 mL of DCM/DMF (1:1 v/v) (50 mL) at room temperature overnight to obtain free acid 13-53. Crude product was purified by column chromatography (1×) to obtain 1.06 g (28%) of purified 13-53. Acid 13-53 (1.06 g, 2 eq., 3.7 mmol) was reacted with dihydroxyacetone (152 mg, 1.0 eq, 1.7 mmol) using EDCI (816 mg, 2.5 eq, 4.25 mmol), DMAP (50 mg, 0.25 eq, 0.43 mmol) and DIPEA (740 μL, 2.5 eq, 4.3 mmol) in 15 mL DCM at room temperature overnight to obtain ketone 13-54. Crude product was purified by column chromatography (1×) to obtain 890 mg (69%) of purified 13-54. Reductive amination of 13-54 (890 mg, 1.0 eq, 1.3 mmol) with amine, 15-3 (327 μl, 2.0 eq, 2.6 mmol) using acetic acid (150 μL, 2.0 eq, 2.6 mmol) and sodium borohydride triacetate, Na(OAc)₃BH (331 mg, 1.2 eq, 1.5 mmol) in 20 mL DCM (20 ml) at room temperature for 3 hours yielded intermediate 13-55. Crude product was purified by column chromatography (1×) to obtain purified 13-55 (470 mg, 47%). N-acylation of intermediate 13-55 using acid 13-31 and reaction conditions reported for N-acylation of Lipid 15 synthesis resulted in Lipid 17.

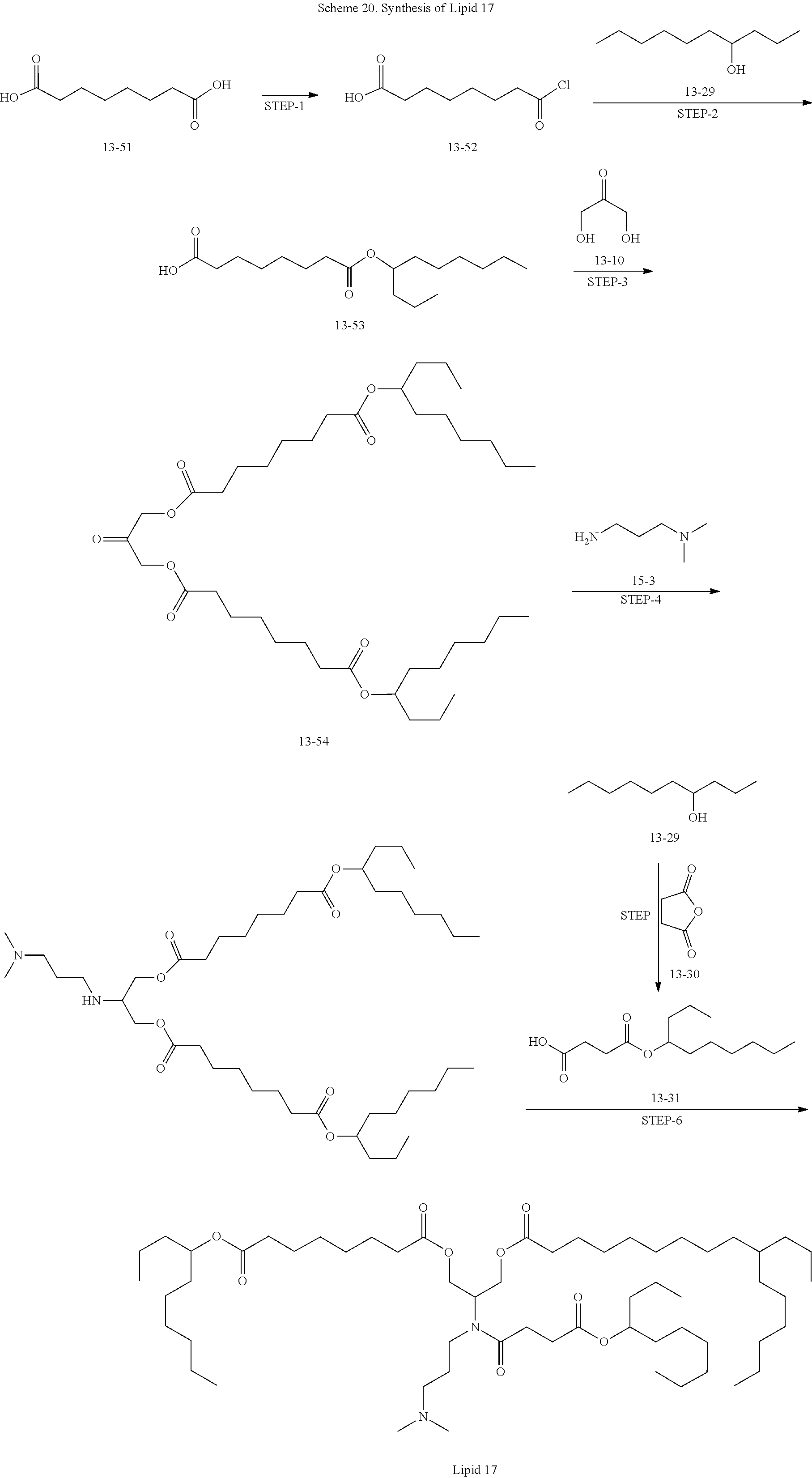

### Synthesis of Lipid 17A

Starting material 4-hydroxydecanol (63.1 mmol, 10 g, 1 eq.) was acylated with succinic anhydride, 13-30 (12.64 g, 2.0 eq, 12.2 mmol) using EDCI (2.65 g, 2.5 eq, 126.3 mmol), DMAP (7.7 g, 1.0 eq, 63.1 mmol) and Pyridine (17 mL) in a mixture of 17 ml of THF and 50 mL of DCM at room temperature, overnight to obtain 8.8 g (540%) of acid intermediate, 13-31. Starting material, 1,3-dihydroxyacetone, 13-10 (8.32 mmol, 0.75 g, 1 eq.) was diacylated with intermediate 13-31 (20.8 mmol, 5.36 g, 2.5 eq) using EDCI (3.98 g, 2.5 eq, 20.8 mmol) and DMAP (0.203 g, 0.2 eq, 1.6 mmol) in 20 mL of DCM at RT, overnight to obtain 1.89 g (40%) of ketone intermediate 13-70. Intermediate 13-70 (3.2 mmol, 31.87 g, 1 eq.) was converted to diamine intermediate 13-71 by reductive amination with N,N-dimethylamino-3-aminopropane, 15-3 (6.5 mmol, 0.66 g, 2.0 eq.) using Na(OAc)₃BH (1.38 g, 2.0 eq, 6.5 mmol), acetic acid (0.37 mL, 2.0 eq, 6.5 mmol) in 15 mL of DCM (15 mL) at room temperature for 3 hours. Purification of crude product by 2× column chromatography (100% MeOH in DCM) yielded 500 mg (23%) of purified Intermediate 13-71. Additional quantity of acid Intermediate 13-31 (19.4 mmol, 0.24 g, 1 eq.) was converted to the corresponding acid chloride 13-31′ using Oxalyl chloride (0.92 mmol, 0.26 mL, 3.4 eq.) and DMF (20 μL, catalytic quantity) in 3 mL of Toluene at room temperature for 2 hours and crude acid chloride 13-31 (0.9 mmol, 0.23 g, 3 eq.) was used for N-acylation of diamine intermediate 13-71 (0.3 mmol, 0.2 g, 1 eq.) using TEA (1.5 mmol, 3.98 g, 5.0 eq) and DMAP (20 mg, catalytic quantity) in 4 mL of Toluene at room temperature overnight to yield Lipid 17A. Crude product was purified 2× by column chromatography (DCM:10% MeOH in DCM) to obtain 165 mg (60%) of pure Lipid 17A (>99% by HPLC-CAD) and characterized by proton NMR and mass spectrometry (FIG. 4W-1 and FIG. 4W-2 for proton NMR and HPLC-CAD and Table 5 for Mass Spectrometry data).

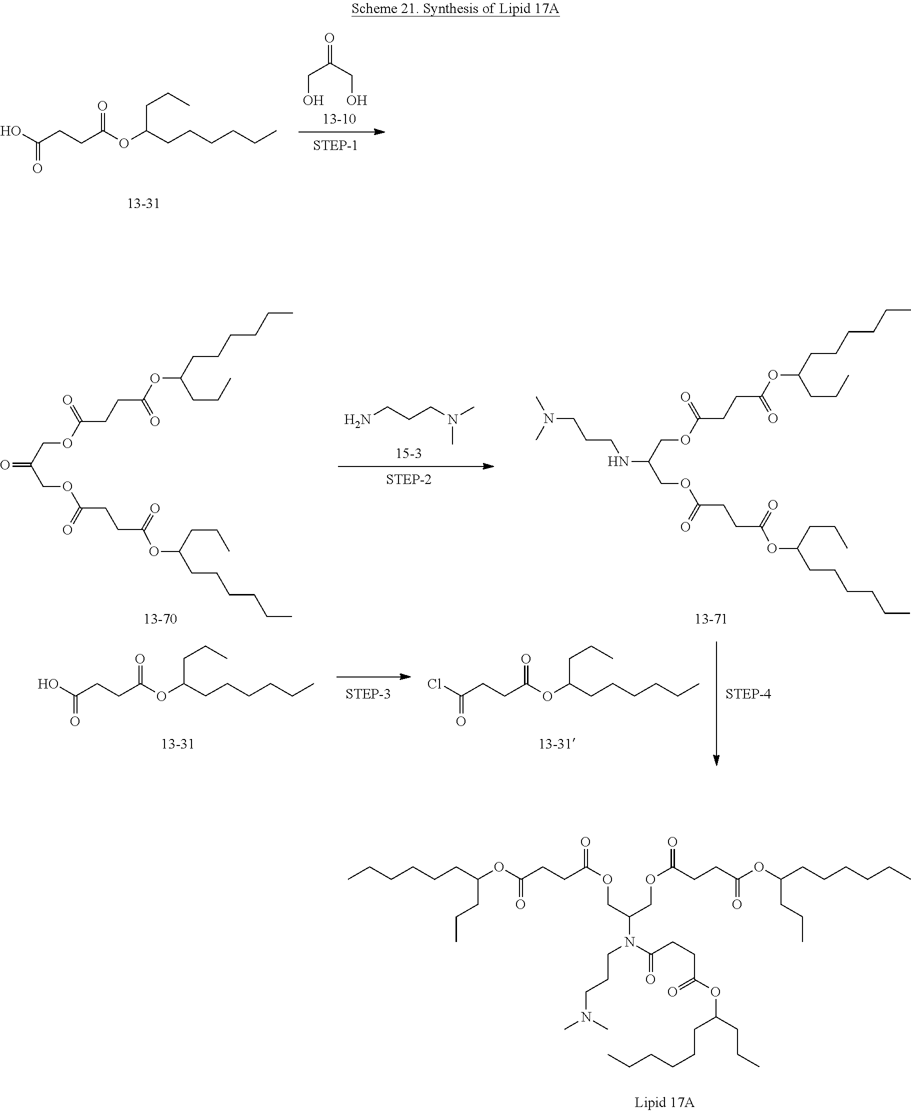

### Synthesis of Lipid 18 and its Isomer

An isomer of lipid 18 was synthesized as provided in scheme 21-1 below. Lipid 18 was synthesis using methods analogous to those reported for Lipid 17 by replacing decan-3-ol with octane-2-ol in the Step 1.

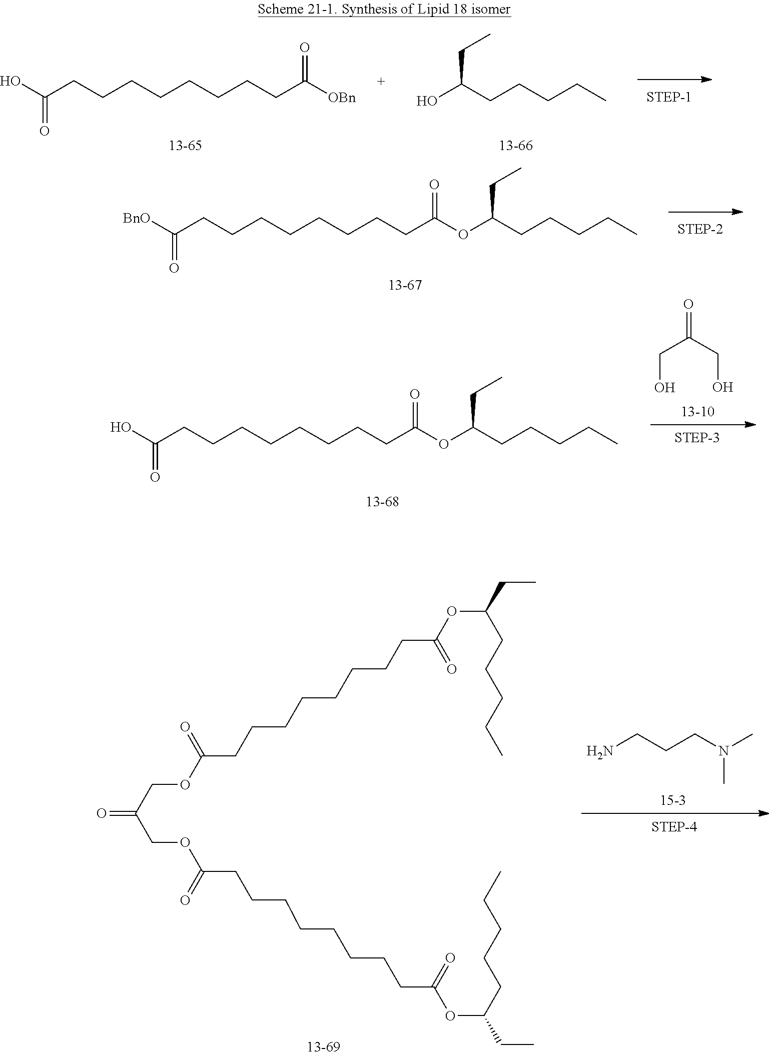

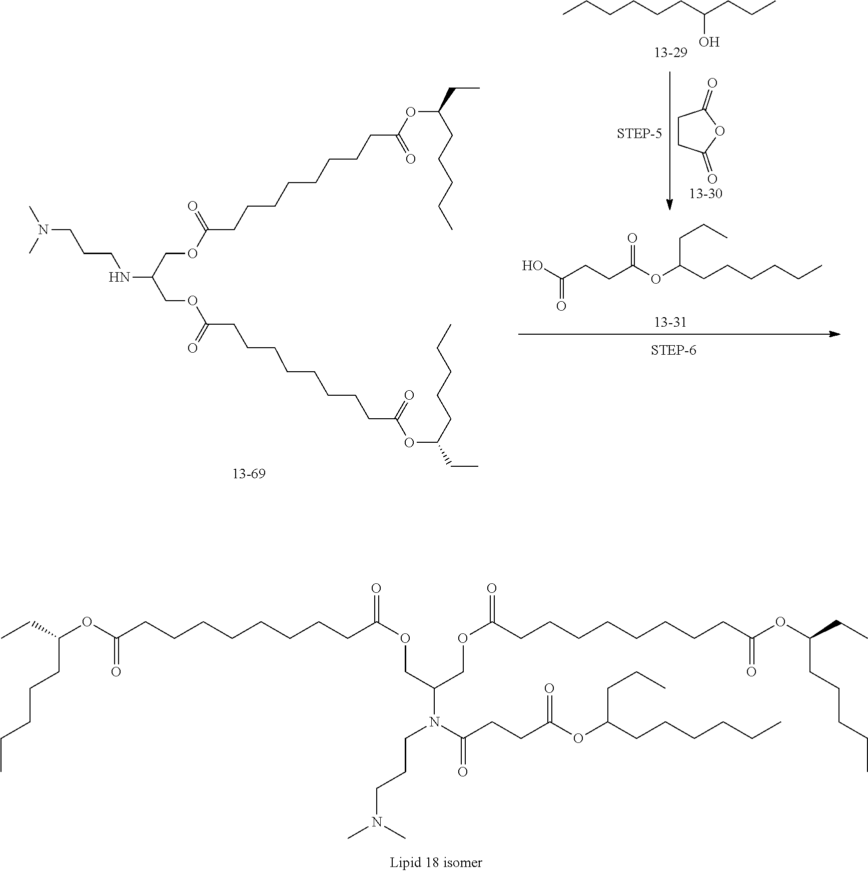 [SMILES: CCCCCCC(O)CCC ; CCCCC[C@H](CC)OC(=O)CCCCCCCCC(=O)OCC(COC(=O)CCCCCCCCC(=O)O[C@@H](CC)CCCCC)NCCCN(C)C ; CCCCCCC(CCC)OC(=O)CCC(=O)O ; O=C1CCC(=O)O1 ; CCCCCCC(CCC)OC(=O)CCC(=O)N(CCCN(C)C)C(COC(=O)CCCCCCCCC(=O)O[C@@H](CC)CCCCC)COC(=O)CCCCCCCCC(=O)O[C@@H](CC)CCCCC]

Lipid 18 as a racemic mixture was synthesized as provided in scheme 21-2 below.

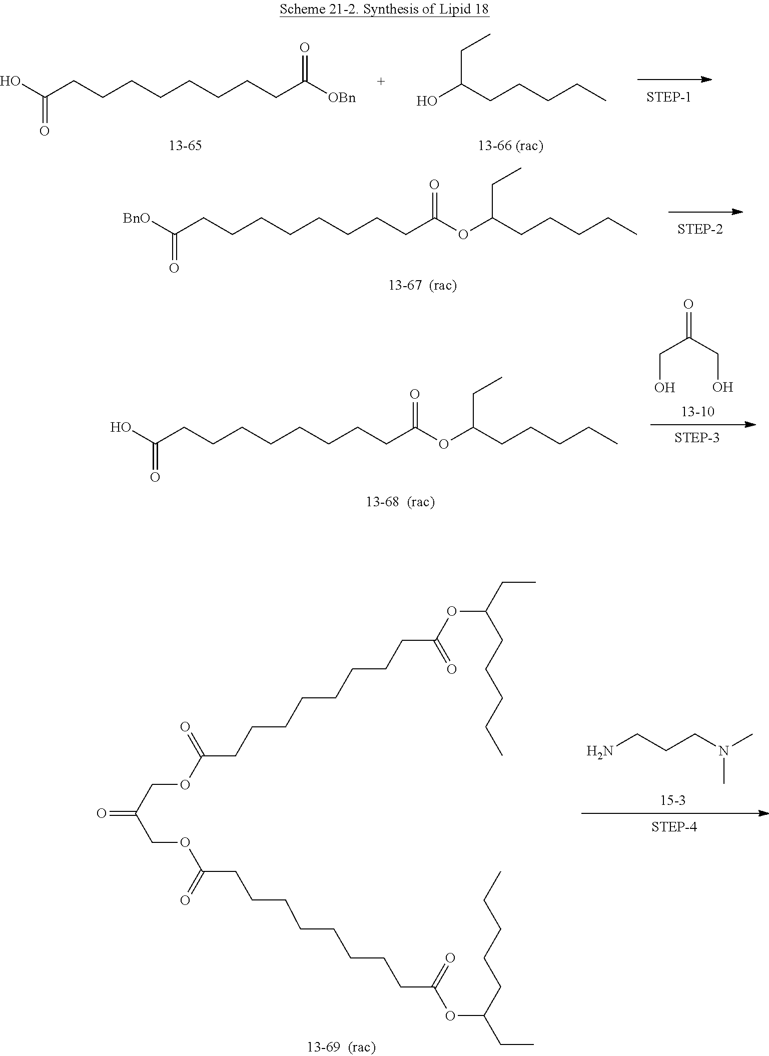

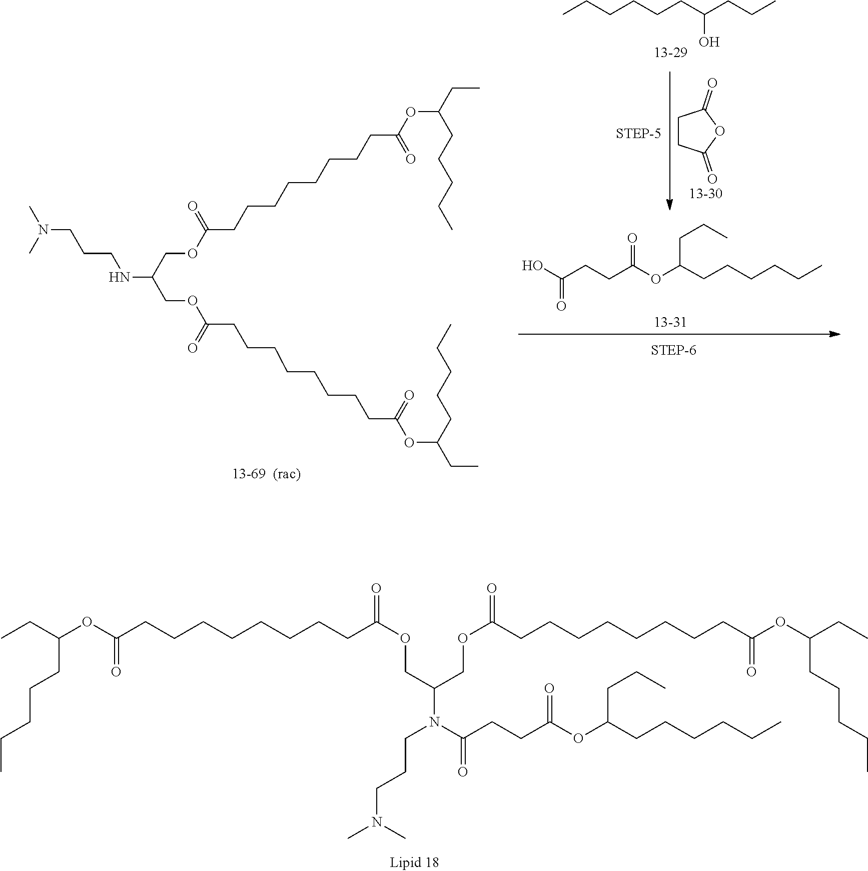 [SMILES: CCCCCCC(O)CCC ; CCCCCC(CC)OC(=O)CCCCCCCCC(=O)OCC(COC(=O)CCCCCCCCC(=O)OC(CC)CCCCC)NCCCN(C)C ; CCCCCCC(CCC)OC(=O)CCC(=O)O ; O=C1CCC(=O)O1 ; CCCCCCC(CCC)OC(=O)CCC(=O)N(CCCN(C)C)C(COC(=O)CCCCCCCCC(=O)OC(CC)CCCCC)COC(=O)CCCCCCCCC(=O)OC(CC)CCCCC]

### Synthesis of Lipid 18A

Starting material 4-hydroxydecanol, 13-29 (31.6 mmol, 5 g, 1 eq.) was acylated with adipic acid, 13-72 (10.4 g, 2.0 eq, 63.2 mmol) using EDCI (37.90 mmol, 7.3 g, 1.2 eq), DMAP (0.5 g, 0.12 eq, 3.8 mmol, 0.5 g, 0.12 eq) and TEA (158 mmol, 22 mL, 5.0 eq.) in a mixture of 50 mL of DCM (50 mL) 50 mL DMF (50 mL) at room temperature, RT, overnight to obtain 9.8 g (90%) of acid intermediate, 13-74. Starting material, 1,3-dihydroxyacetone, 13-10 (11.09 mmol, 1.0 g, 1.0 eq.) was diacylated with acid Intermediate 13-74 (27.7 mmol, 7.93 g, 2.5 eq.) using EDCI (27.7 mmol, 5.32 g, 2.5 eq,) and DMAP (3.58 g, 0.2 eq, 2.2 mmol) in 30 mL at room temperature overnight to obtain 1.18 g (17%) of ketone intermediate 13-75. Intermediate 13-75 (3.2 mmol, 1.16 g, 1.0 eq.) was converted to diamine intermediate 13-76 by reductive amination with N,N-dimethylamino-3-aminopropane, 15-3 (6.5 mmol, 0.66 g, 2.0 eq.) using Na(OAc)₃BH (1.38 g, 2.0 eq, 6.5 mmol), acetic acid (0.37 mL, 2.0 eq, 6.5 mmol) in 15 mL of DCM (15 mL) at room temperature for 3-4 hours. Purification of crude product by 2× column chromatography (10% MeOH in DCM) yielded 660 mg (50%) of purified Intermediate 13-76. Additional quantity of acid Intermediate 13-31 (19.4 mmol, 0.24 g, 1 eq.) was converted to the corresponding acid chloride 13-31′ using Oxalyl chloride (0.92 mmol, 0.26 mL, 3.4 eq.) and DMF (20 μL, catalytic quantity) in 3 mL of Toluene at room temperature for 2 hours and crude acid chloride 13-31 (0.9 mmol, 0.23 g, 3 eq.) was used for N-acylation of diamine intermediate 13-76 (0.3 mmol, 0.2 g, 1 eq.) using TEA (1.5 mmol, 3.98 g, 5.0 eq) and DMAP (20 mg, catalytic quantity) in 4 mL of Toluene at room temperature overnight to yield Lipid 18A. Crude product was purified 2× by column chromatography (DCM:10% MeOH in DCM) to obtain 175 mg (60%) of pure Lipid 18A (>99% by HPLC-CAD) and characterized by proton NMR and mass spectrometry (FIG. 4X-1 and FIG. 4X-2 for proton NMR and HPLC-CAD and Table 5 for Mass Spectrometry data).

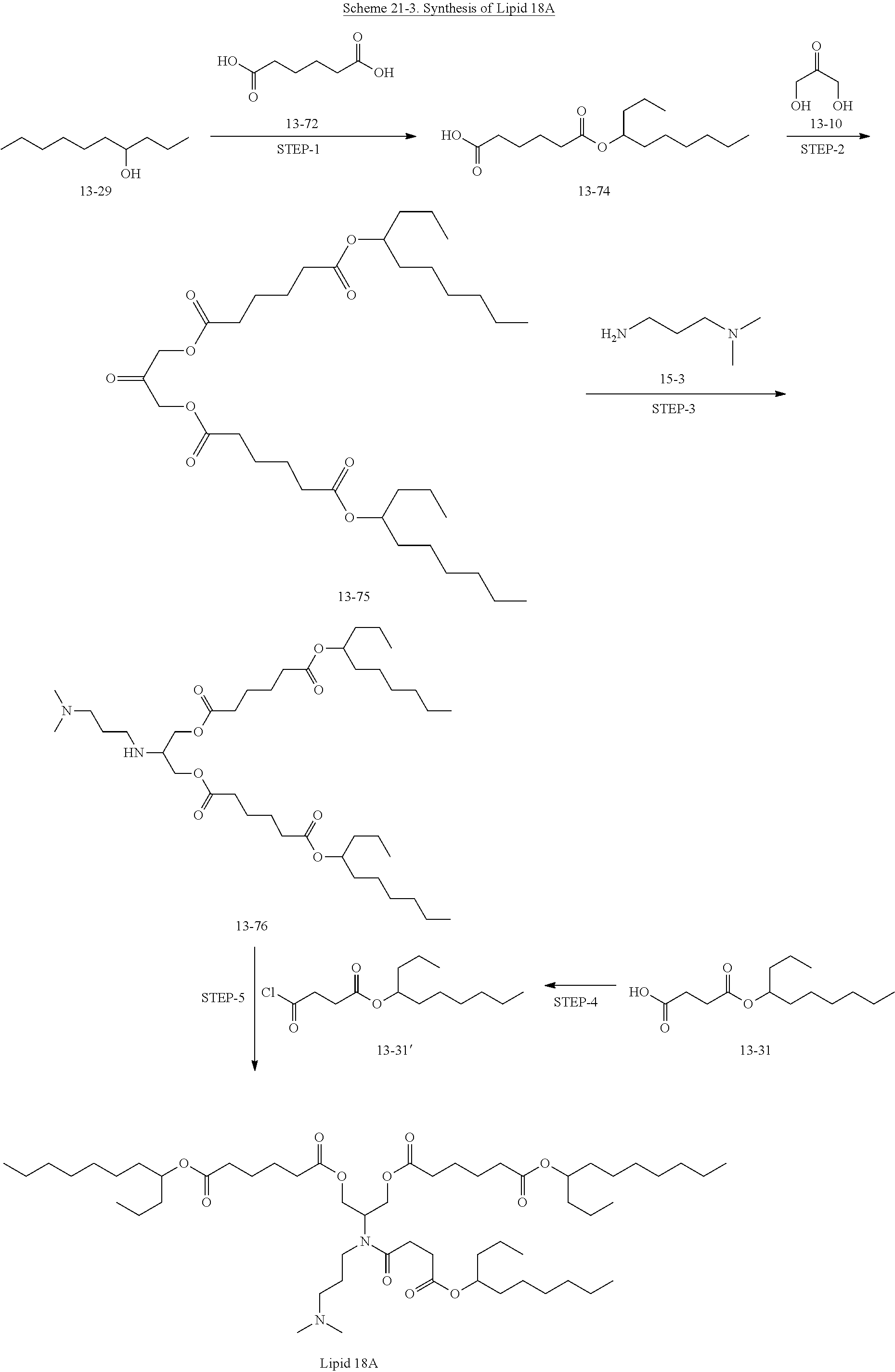

### Synthesis of Lipid 19

Lipid 19 was synthesized as provided in scheme 22 below and as follows. Starting material dihydroxyacetone (422 mg, 4.7 mmol) was acylated with compound 13-56 (3.0 g, 2.5 eq, 11.71 mmol) using EDCI (2.24 g, 2.5 eq, 11.71 mmol), DIPEA (2.0 mL, 2.5 eq, 11.71 mmol), and DMAP (115 mg, 0.2 eq, 0.94 mmol) in 10 mL DCM yielding 2.1 g (79 d) of intermediate 13-57. Reductive amination of 13-57 (2.1 g, 1.0 eq, 3.7 mmol) with amine 15-3 (925 μL, 2.0 eq, 7.4 mmol) using acetic acid (430 μL, 2.0 eq, 7.4 mmol), Na(OAc)₃BH (923 mg, 1.2 eq, 4.44 mmol) in 10.0 mL DCM yielding 1.55 g (65%) of intermediate 13-58. Intermediate 13-31 was produced as described in the synthesis of Lipid 9 and Lipid 15 earlier. N-acylation of intermediate 13-58 (484 mg, 1.0 eq, 0.74 mmol) with 13-31 (380 mg, 2.0 eq, 1.48 mmol) using EDCI (291 mg, 2.0 eq, 1.48 mmol), DIPEA (247 μL, 2.0 eq, 1.48 mmol), and DMAP (45 mg, 0.5 eq, 0.37 mmol) in 4.0 mL DCM at room temperature, overnight yielded 423 mg (63%) of pure lipid 19 (>99% purity).

See FIG. 4P-1 for Lipid 19 NMR spectrum, FIG. 4P-2 for Lipid 19 reverse phase LC-ELSD chromatogram, and Table 5 for product mass.

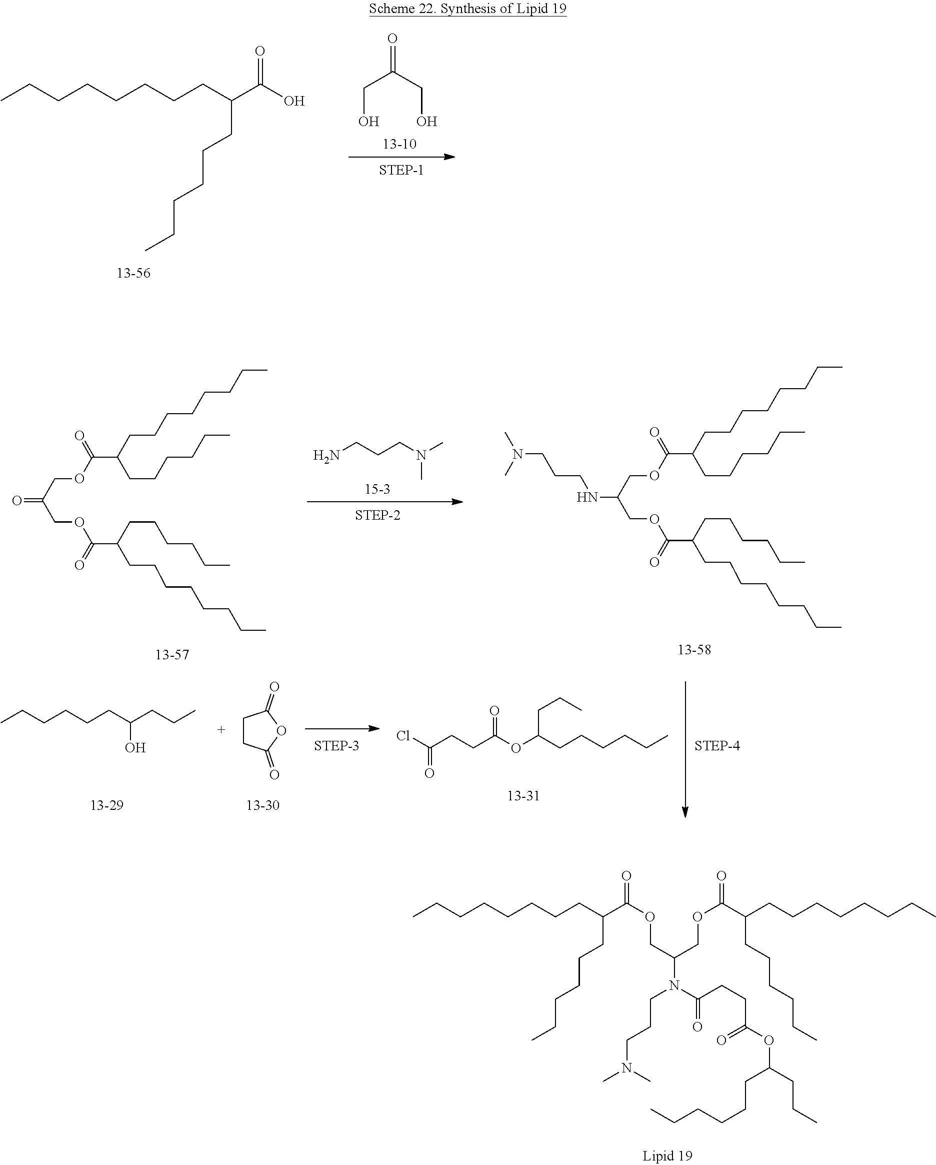

### Synthesis of Lipid 19A

Starting material tertiary-butyl protected suberic acid 13-51 (8.6 mmol, 2.0 g, 1 eq.) was esterified using 4-hydroxydecanol 13-52 (13.02 mmol, 2.06 g, 1.5 eq) using EDCI (13.02 mmol, 2.49 g, 1.5 eq), DMAP (4.3 mmol, 0.53 g, 0.5 eq) and DIPEA (13.02 mmol, 1.68 g, 1.5 eq) in 20 mL of dichloromethane at room temperature for 4 hours. yielding 2.83 g (88%) of protected intermediate 13-53. Intermediate 13-53 (4.05 mmol, 1.5 g, 1.0 eq.) was deprotected using 4N HCl in 10 mL dioxane at room temperature overnight yielding 1.07 g (84%) of acid intermediate 13-54.

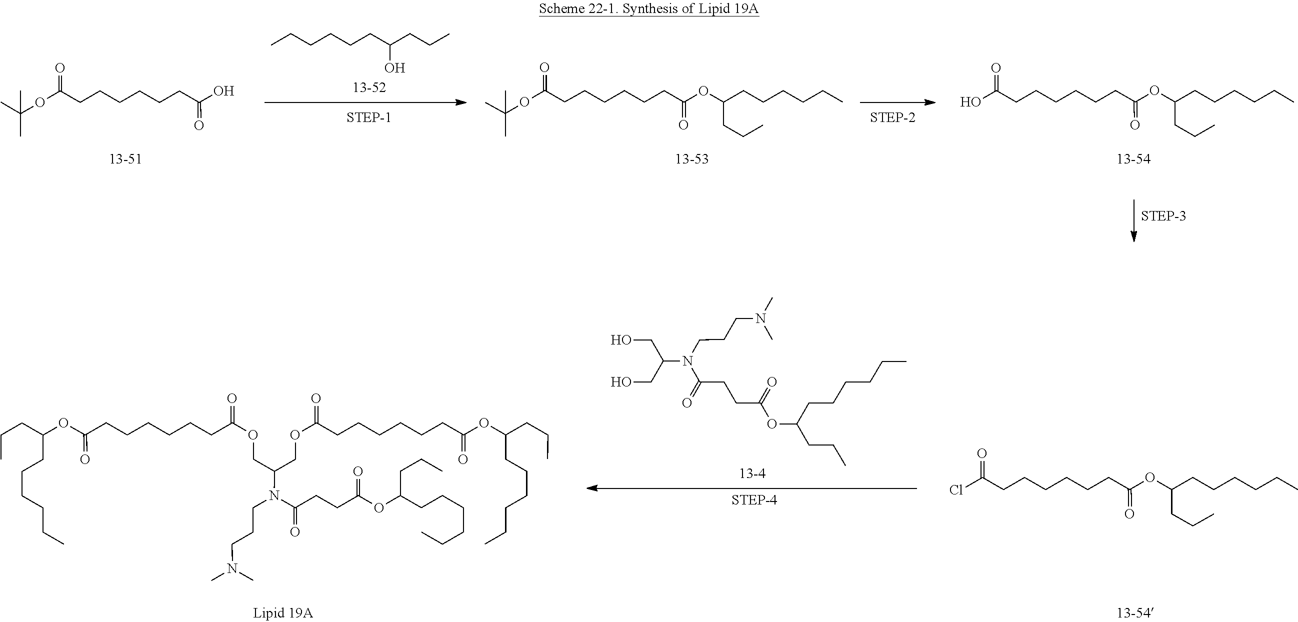

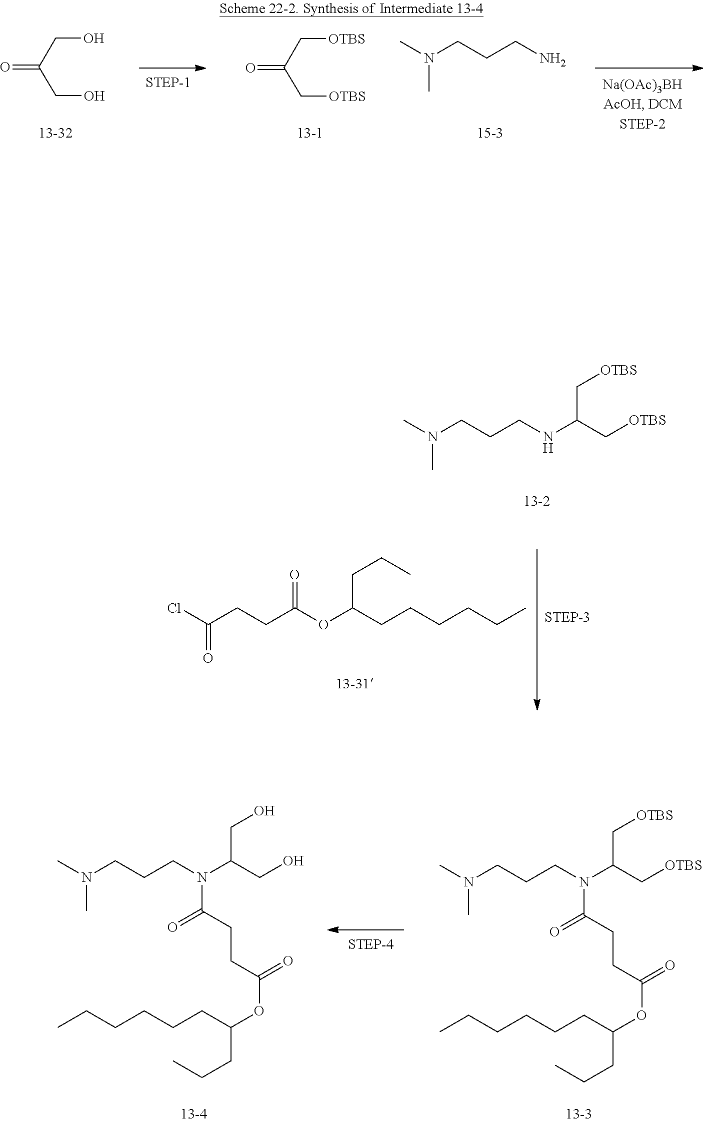

Protection of starting material dihydroxyacetone 13-32 (111 mmol, 10 g, 1 eq.) using tert-butyltrimethylsilyl chloride TBSCl (332 mmol, 50 g, 3.0 eq), TEA (148 mmol, 160 mL, 10.34 eq) and DMAP (23 mmol, 2.80 g, 0.21 eq.) in 420 mL DCM (420.0 mL) at room temperature overnight yielded protected intermediate 13-1. A second batch of 13-1 was produced from 50 g (0.56 mol, 1.0 eq) of additional 13-32 using TBSCl (1.68 mol, 250 g, 3.0 eq), TEA (5.6 mol, 400 mL, 10.34 eq,), DMAP (0.11 mol, 13.7 g, 0.21 eq) in 800 mL of DCM at room temperature overnight. Crude products from the two batches were reductively aminated separately and combined prior to purification. First batch of 13-1 (176 g, 552.0 mmol) was reductively aminated using N,N-dimethylaminopropyl amine 15-3, (1104.0 mmol, 139 mL, 2.0 eq.), Acetic acid (1104.0 mmol, 64 mL, 2.0 eq.), and Na(OAc)₃BH (662.0 mmol, 135 g, 1.2 eq.), in 1.5 L of dichloromethane at room temperature for 3 hours. The second batch of 13-1 (36.8 g, 115.4 mmol) was reductively aminated using N,N-dimethylaminopropyl amine 15-3, (230.8 mmol, 29 mL, 2.0 eq.), Acetic acid (230.8 mmol, 13.4 mL, 2.0 eq), and Na(OAc)₃BH (138.5 mmol 28.2 g, 1.2 eq.), in 300 mL of dichloromethane at room temperature for 3 hours. Combined crude product from the two batches was purified by filter column chromatography on silica column eluting with DCM and (10% MeOH in DCM+1% NH₄OH) to obtain desired product yielding 17 g pure intermediate 13-2 based on TLC.

Two batches of 13-2 were separately deprotected using identical reactions conditions; each consisting of 13-2 (300 mg, 0.465 mmol) in HF-pyridine, (4.65 mmol, 0.42 mL, 10.0 eq.) and 2 mL THE at room temperature for 2 hours.

Two batches of acid intermediate 13-54 were separately converted to the corresponding acid chloride using identical reactions conditions; each consisting of 13-54 (881 mg, 2.8 mmol) using oxalyl chloride (9.5 mmol, 0.8 mL, 3.4 eq), DMF (100 μL, catalytic quantity) in 6.0 mL of toluene at room temperature for 2 hours.

Crude di-hydroxy intermediate 13-4 (194 mg, 0.465 mmol) and crude acid chloride 13-54′ (2.8 mmol, 881 mg, 6.0 eq.), were combined with TEA (4.65 mmol, 0.65 mL, 10.0 eq) in 8.0 mL of toluene at room temperature overnight. Crude product was purified on ISCO column chromatography on silica column eluting with DCM and 10% MeOH in DCM. Column purification was repeated after product isolation yielding 247 mg (53%) of 76% pure (HPLC-CAD) Lipid 19A. Product was re-purified on ISCO column chromatography on silica column eluting with DCM and 10% MeOH in DCM. yielding 122 mg of >99% purity (HPLC-CAD) Lipid 19A (see FIG. 4AI-1 and FIG. 4AI-2 for characterization by proton NMR, and LC-CAD purity and Table 5 for Mass Spectrometry data).

### Synthesis of Lipid 20

Lipid 20 was synthesized as provided in scheme 23 below and as follows. Monoprotected succinic acid, 13-59 (2.0 g, 1.0 eq, 9.65 mmol) was reduced to the corresponding alcohol using Borane-dimethyl sulfide (6.2 mL, 7.0 eq, 67.0 mmol) at 0-5° C., 1 hr followed by room temperature reaction overnight. Crude product was purified by column chromatography (2×) yielding 1.3 g (71%) of pure compound 13-60. Intermediate 13-60 (1.3 g, 1.3 eq, 6.7 mmol) was used to acylate acid 13-56 (1.51 mL, 1.0 eq, 5.0 mmol) using EDCI (1.63 g, 1.7 eq, 8.5 mmol), DIPEA (1.48 mL, 1.7 eq, 8.5 mmol) and DMAP (98 mg, 0.17 eq, 0.85 mmol) in 10.0 mL DCM at room temperature overnight. Crude product was purified by column chromatography (1×) yielding 1.88 g (65%) of pure intermediate 13-61. Subsequent deprotection by hydrogenation on Pd/C/Hydrogen gas (400 mg) in methanol yielded 1.42 g of free acid 13-62 (99%) of crude product. Crude 13-62 (1.32 g, 2.2 eq, 4.2 mmol) was used to acylate dihydroxyacetone, 13-10 (172 mg, 1.0 eq, 1.9 mmol), EDCI (958 mg, 2.6 eq, 5.0 mmol), DIPEA (870 μL, 2.6 eq, 5.0 mmol), and DMAP (56 mg, 0.26 eq, 0.5 mmol) in 10.0 mL DCM at room temperature, overnight to obtain ketone 13-63. Crude product was purified by column chromatography to obtain 120 mg (3.8%) of pure 13-63. Reductive amination of 13-63 (120 mg, 1.0 eq, 0.16 mmol) with amine 15.-3 (42 μl, 2.0 eq, 0.32 mmol) using acetic acid (18 μL, 2.0 eq, 7.8 mmol) and Na(OAc)₃BH (41 mg, 1.2 eq, 0.19 mmol) in 3 mL DCM at room temperature of 3 hours yielded intermediate 13-64. Crude product was purified by column chromatography (1×) to obtain 23 mg (17%) of purified intermediate 13-64. N-acylation of 13-64 (23 mg, 1.0 eq, 0.028 mmol) with acid 13-31 (8.7 mg, 1.2 eq, 0.034 mmol) using EDCI (6.4 mg, 1.2 eq, 0.034 mmol), DIPEA (5.8 μL, 1.2 eq, 0.034 mmol) and DMAP (1 mg, cat) in 1.5 mL DCM at room temperature overnight yielded Lipid 20. Crude product was purified by column chromatography (1×) to obtain 21 mg (70%) of pure lipid 20 (99%).

See FIG. 4Q-1 for Lipid 20 NMR spectrum, FIG. 4Q-2 for Lipid 20 reverse phase LC-ELSD chromatogram, and Table 5 for product mass.

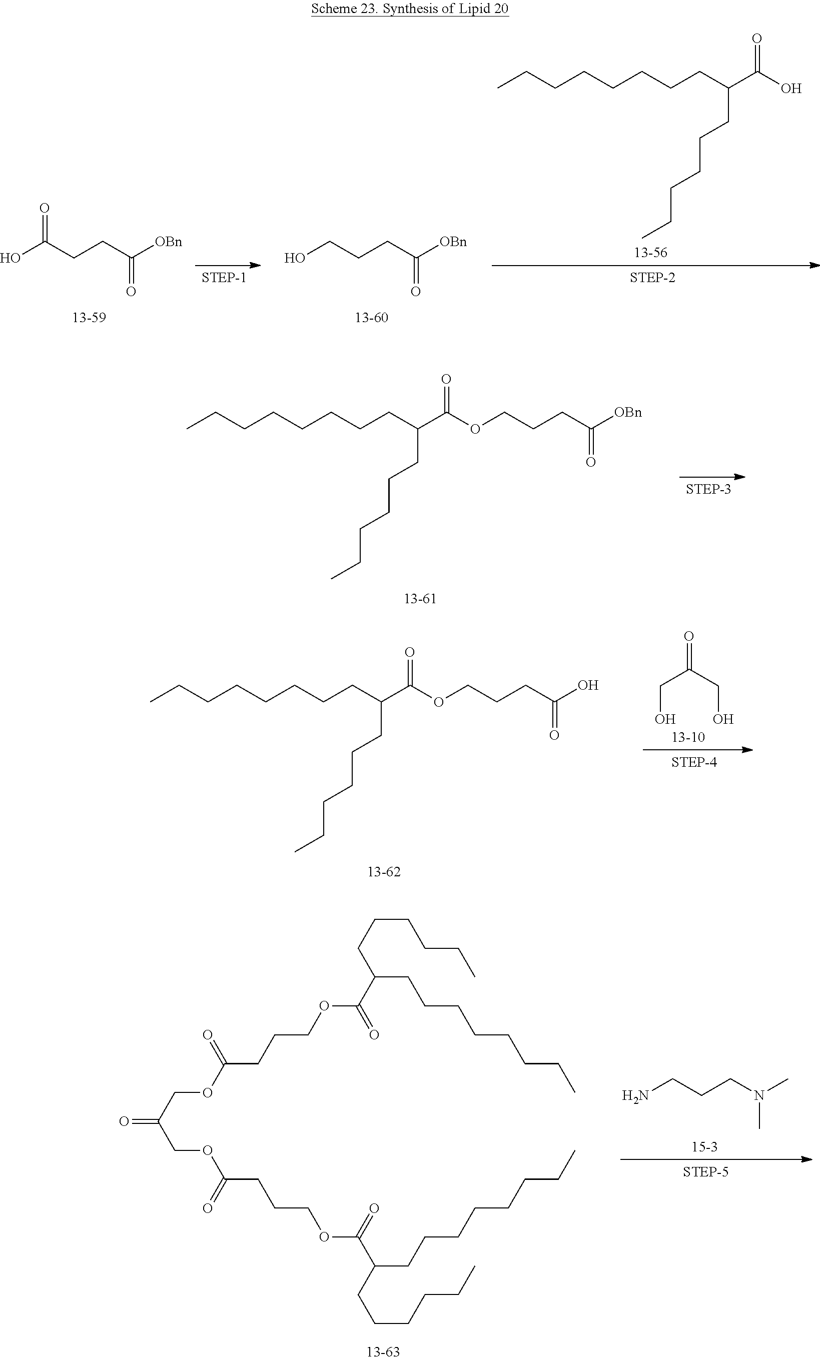

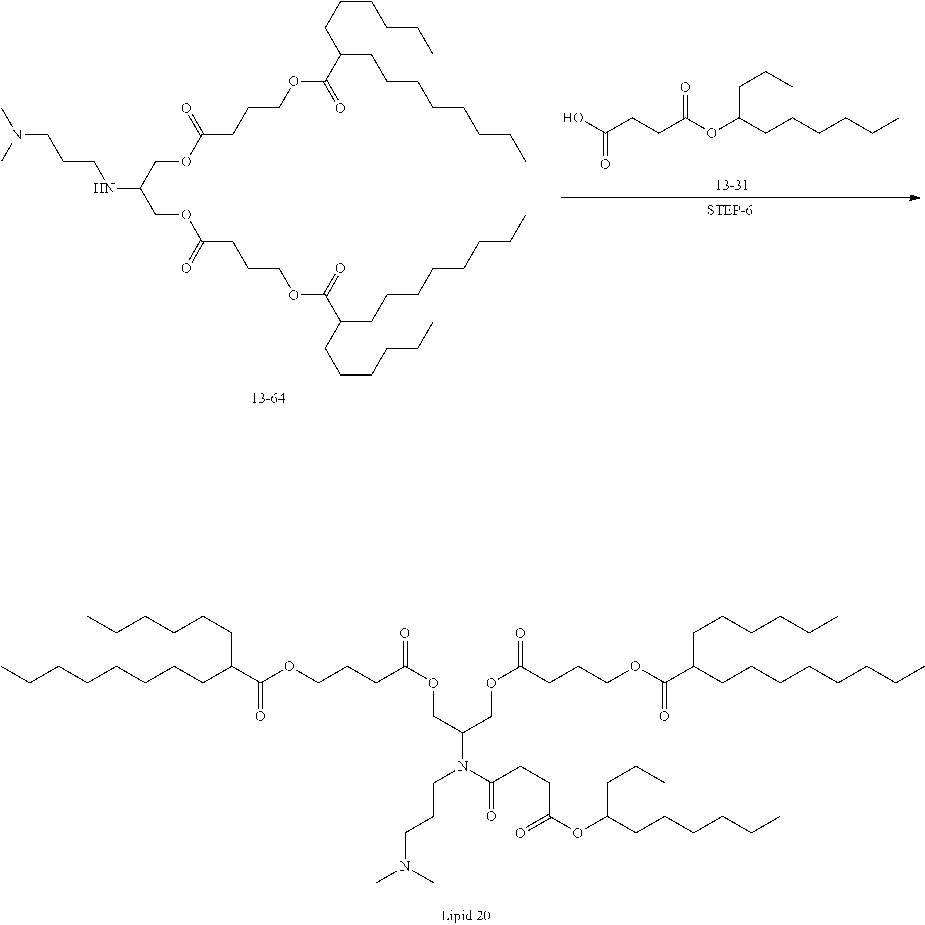 [SMILES: CCCCCCCCC(CCCCCC)C(=O)OCCCC(=O)OCC(COC(=O)CCCOC(=O)C(CCCCCC)CCCCCCCC)NCCCN(C)C ; CCCCCCC(CCC)OC(=O)CCC(=O)O ; CCCCCCCCC(CCCCCC)C(=O)OCCCC(=O)OCC(COC(=O)CCCOC(=O)C(CCCCCC)CCCCCCCC)N(CCCN(C)C)C(=O)CCC(=O)OC(CCC)CCCCCC]

### Synthesis of Lipid 20A

Starting material tertiary-butyl protected sebacic acid 14-19 (15.5 mmol, 4.0 g, 1 eq.) was esterified using 4-hydroxydecanol 14-20 (23.25 mmol, 3.7 g, 1.5 eq) and EDCI (23.25 mmol, 4.5 g, 1.5 eq), DMAP (7.75 mmol, 0.95 g, 0.5 eq) and DIPEA (23.25 mmol, 4 mL, 1.5 eq.) in 20 mL of dichloromethane at room temperature overnight yielding 4.1 g (66%) of protected intermediate ester 14-21. Intermediate 14-21 (10.3 mmol, 4.1 g, 1.0 eq.) was deprotected using 4N HCl in 15 mL dioxane at room temperature overnight yielding 2.7 g (77%) of acid intermediate 14-22.

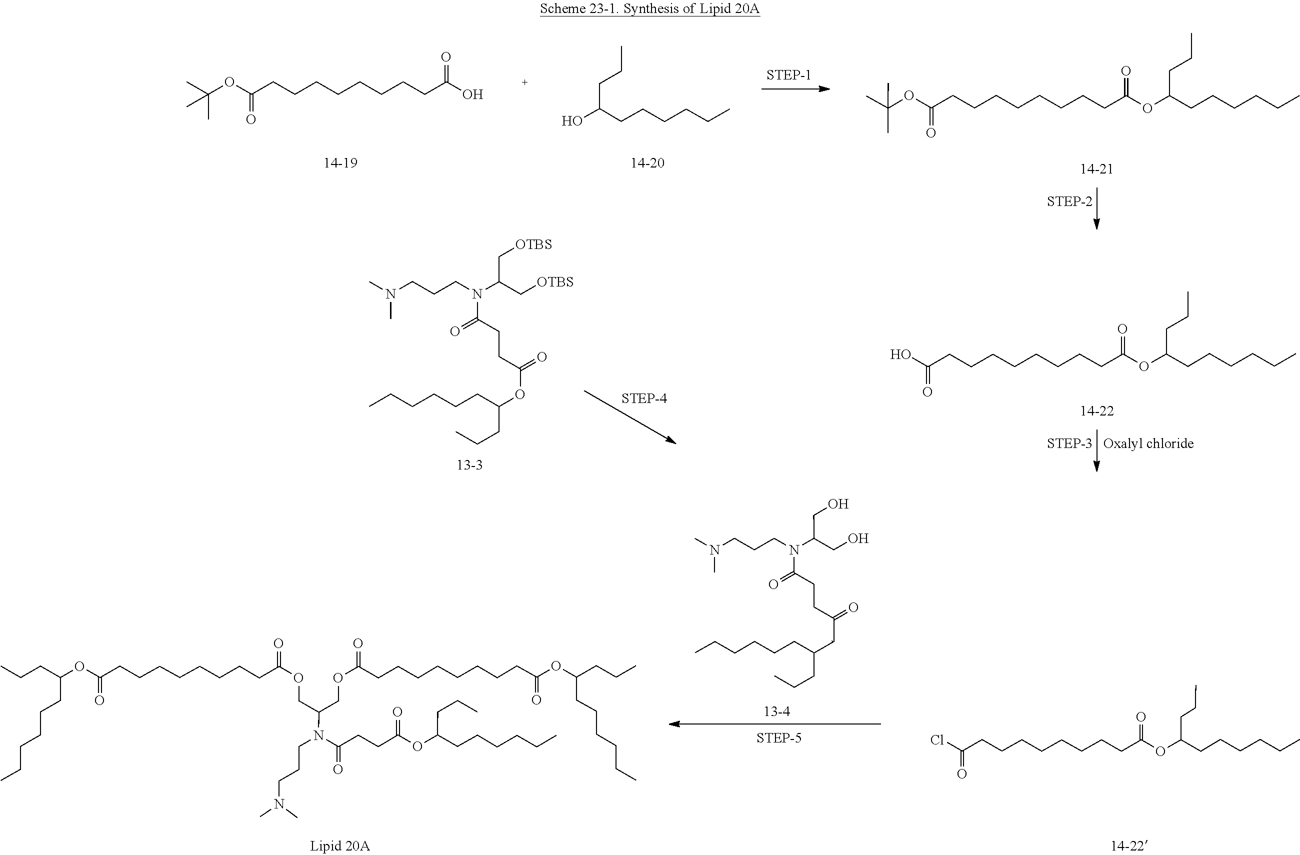

Protected intermediate 13-3 (400 mg, 0.62 mmol, 1 eq.) was treated with Hydrogen Fluoride/Pyridine (15.5 mmol, 1.11 mL, 25.0 eq.) in 6.0 mL THF at room temperature for 2 hours and deprotection was confirmed by TLC and mass spectrometry. Acid intermediate 14-22 (3.72 mmol, 1.27 g, 1 eq.) was converted to the corresponding acid chloride 14-22′ using oxalyl chloride (12.6 mmol, 1.1 mL, 3.4 eq.) and DMF (40 μL, catalytic quantity) in 5.0 mL of Toluene at room temperature for 2 hours and conversion to chloride intermediate by TLC.

Crude di-hydroxy intermediate 13-4 (258 mg, 0.62 mmol, 1 eq.) and crude acid chloride 14-22′ (3.72 mmol, 1.34 g, 6.0 eq.) were combined with TEA (6.2 mmol, 0.87 mL, 10.0 eq) in 5 mL of toluene at room for 2 hours. Crude product was purified on ISCO column chromatography on silica column eluting with DCM and 10% MeOH in DCM yielding 300 mg of 96% pure (HPLC-CAD) Lipid 20A (see FIG. 4AJ-1 and FIG. 4AJ-2 for characterization by proton NMR, mass spectrometry and LC-CAD purity).

### Synthesis of Lipid 21 and its Isomer

An isomer of lipid 21 was synthesized as provided in scheme 24-1 below. Briefly, alcohol 13-78 was accessed by nucleophilic addition to aldehyde 13-77 using diethyl zinc (Step 1) and subsequently used in the ring opening addition to cyclic anhydride 13-52 to access intermediate 13-79. O-acylation of dihydroxyacetone with intermediate 13-79 using conditions described in Lipid 17 synthesis yielded ketone 13-80. Reductive amination of 13-80 with amine 15-3 using conditions described in the Lipid 17 synthesis yielded intermediate 13-81. Subsequent N-acylation of intermediate 13-81 with acid 13-31 using conditions analogous to those used for Lipid 9 synthesis afforded Lipid 21.

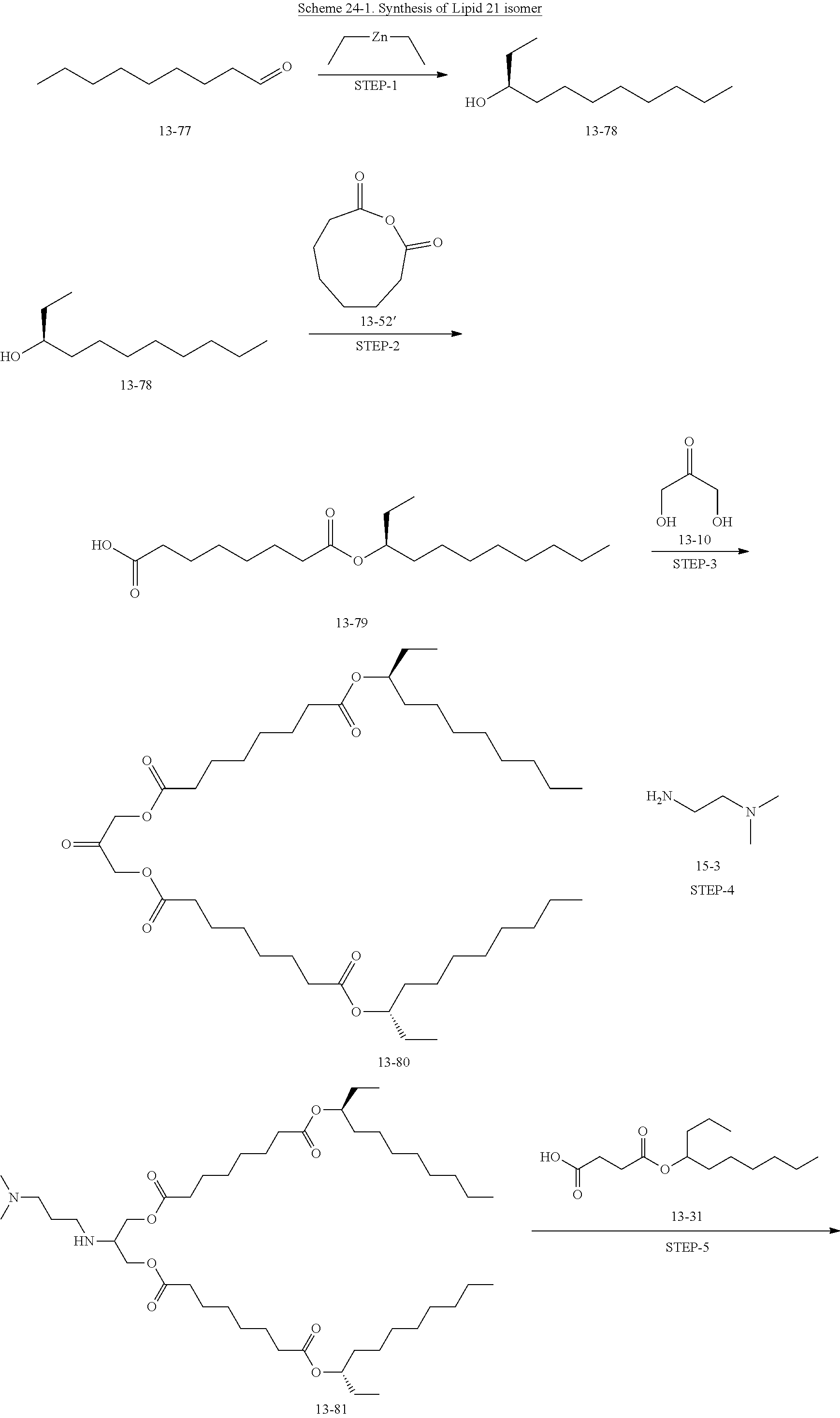

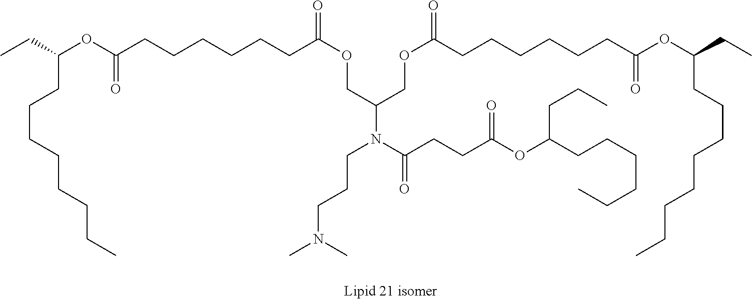 [SMILES: CCCCCCCC[C@H](CC)OC(=O)CCCCCCC(=O)OCC(COC(=O)CCCCCCC(=O)O[C@@H](CC)CCCCCCCC)N(CCCN(C)C)C(=O)CCC(=O)OC(CCC)CCCCCC]

Lipid 21 as a racemic mixture was synthesized as provided in scheme 24-2 below. Briefly, Lipid 21 (racemate) was accessed using methods analogous to those described for Lipid 21 isomer except using ethyl lithium for accessing the racemic alcohol in Step 1.

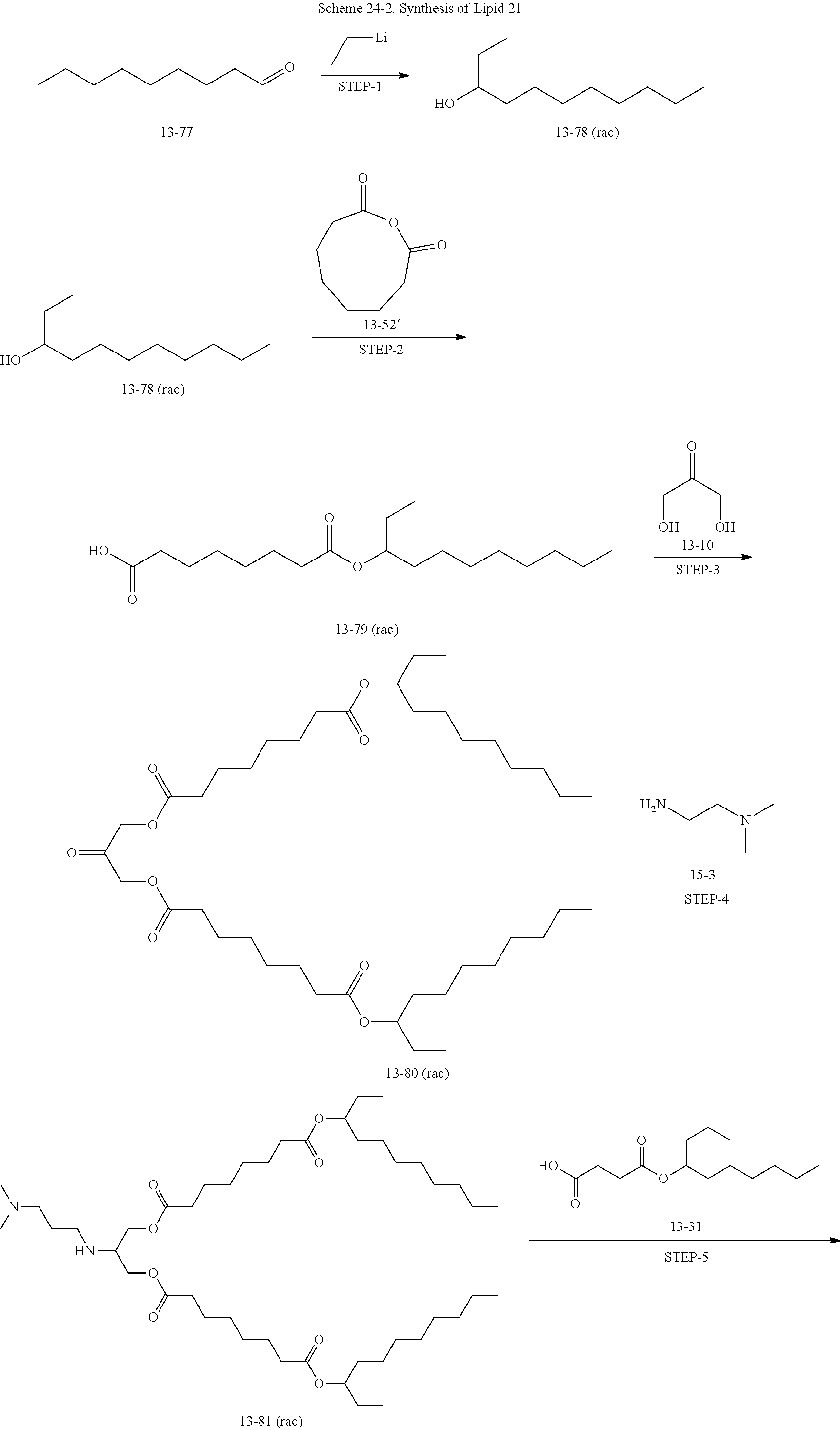

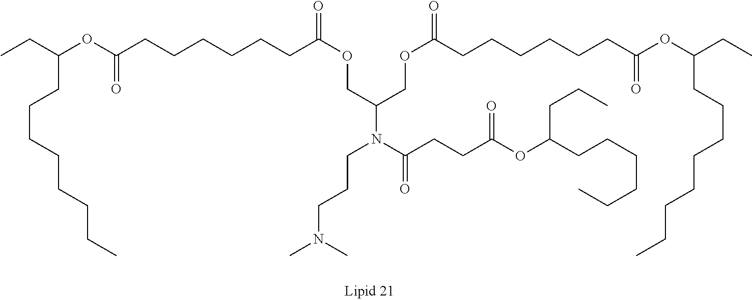 [SMILES: CCCCCCCCC(CC)OC(=O)CCCCCCC(=O)OCC(COC(=O)CCCCCCC(=O)OC(CC)CCCCCCCC)N(CCCN(C)C)C(=O)CCC(=O)OC(CCC)CCCCCC]

### Synthesis of Lipid 21A

Starting material 3-hydroxyoctanol, 13-66 (12.4 mmol, 1.61 g, 1 eq.) was acylated with sebacic acid 13-65 (24.8 mmol, 5.0 g, 2.0 eq.) using EDCI (14.9 mmol, 2.83 g, 1.2 eq.), DMAP (1.5 mmol, 183 mg, 0.12 eq.) and TEA (62.0 mmol, 8.6 mL, 5.0 eq.) in a mixture of 25 mL of DCM and 25 mL DMF at room temperature, RT, overnight to obtain 2.2 g (56%) of acid intermediate, 13-67. Starting material, 1,3-dihydroxyacetone, 13-10 (3.2 mmol, 286 mg, 1.0 eq.) was diacylated with acid Intermediate 13-67 (7.0 mmol, 2.2 g, 2.2 eq.) using EDCI (7.0 mmol, 1.33 g, 2.2 eq.), DIPEA (7.0 mmol, 1.22 mL, 2.2 eq.) and DMAP (1.6 mmol, 197 mg, 0.5 eq.) in 10 mL of DCM at room temperature overnight to obtain 1.4 g (65%) of ketone intermediate 13-68.

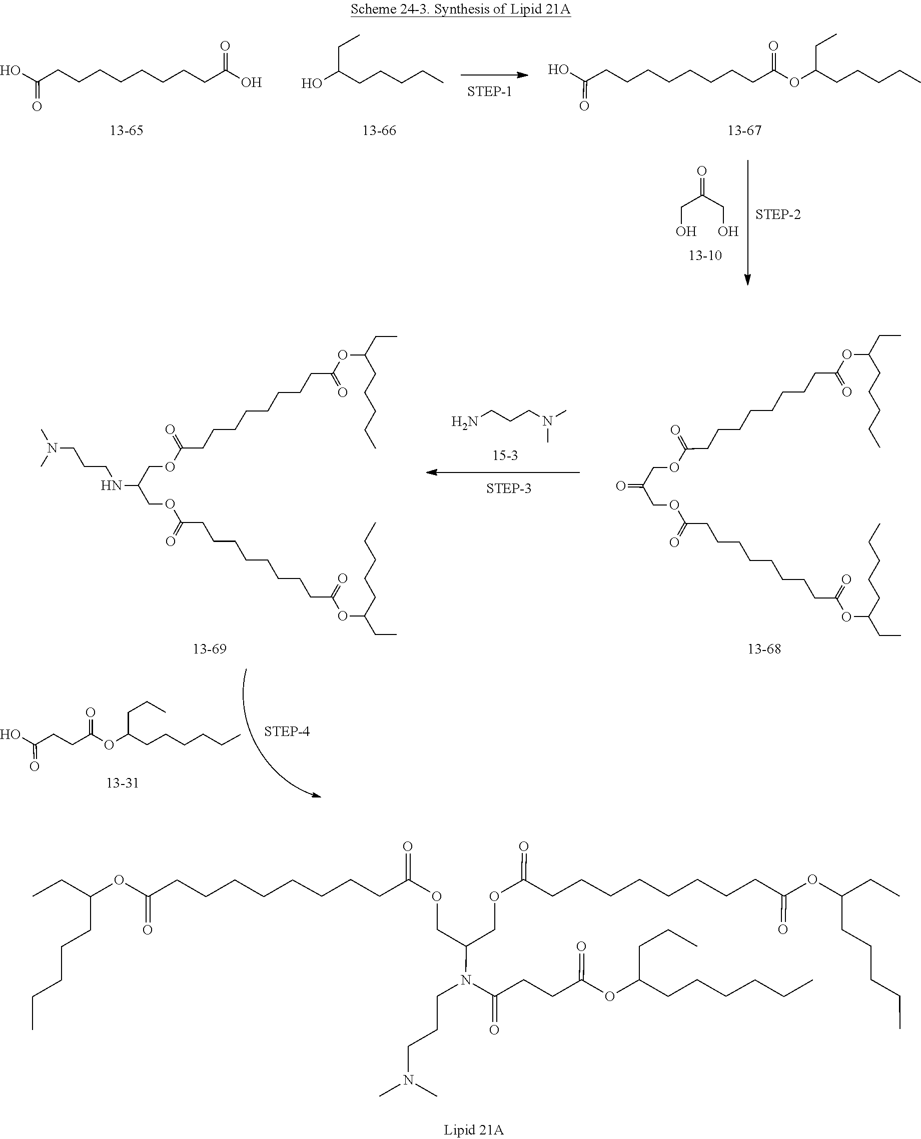

Intermediate 13-68 (2.05 mmol, 1.4 g, 1.0 eq.) was converted to diamine intermediate 13-69 by reductive amination with N,N-dimethylamino-3-aminopropane, 15-3 (514 μL, 2.0 eq, 4.10 mmol) using Na(OAc)₃BH (10 mg, 1.2 eq, 2.46 mmol)), acetic acid (236 μL, 2.0 eq, 4.10 mmol) in 5 mL of DCM at room temperature for 3 hours. Purification of crude product by silica column chromatography (10% MeOH in DCM, 1% NH₄OH) yielded 480 mg (32%) of purified Intermediate 13-69. Acid Intermediate 13-31 (1.86 mmol, 484 mg, 1 eq.) was converted to the corresponding acid chloride 13-31′ using Oxalyl chloride (540 μL, 3.4 eq, 6.38 mmol) and DMF (20 μL, catalytic quantity) in 3 mL of Toluene at room temperature for 2 hours and crude acid chloride 13-31′, (518 mg, 3.0 eq, 1.88 mmol), TEA, Toluene (3.0 mL), RT, Overnight was used for N-acylation of diamine intermediate 13-69 (0.3 mmol, 0.2 g, 1 eq.) using TEA (435 μL, 5.0 eq, 3.13 mmol) and in 3 mL of Toluene at room temperature overnight to yield Lipid 21A. Crude product was purified 2× by column chromatography (Hexanes and EtAc, then DCM:10% MeOH in DCM on silica) to obtain 51 mg of pure Lipid 21A (>99% by HPLC-UV and 95% by HPLC-CAD) and characterized by proton NMR and mass spectrometry (FIG. 4Y-1 and FIG. 4Y-2 for proton NMR and HPLC-CAD and Table 5 for Mass Spectrometry data).

### Synthesis of Lipid 22 and its Isomer

An isomer of lipid 22 was synthesized as provided in scheme 25-1 below. Briefly, alcohol 13-78 (accessed as described for Lipid 21 synthesis above) was used in the ring opening addition to cyclic anhydride 13-73′ to access intermediate 13-82. O-acylation of dihydroxyacetone with intermediate 13-82 using conditions described in Lipid 17 synthesis yielded ketone 13-83. Reductive amination of 13-83 with amine 15-3 using conditions described in the Lipid 17 synthesis yielded intermediate 13-84. Subsequent N-acylation of intermediate 13-84 with acid 13-31 using conditions analogous to those used for Lipid 9 synthesis afforded Lipid 22 isomer.

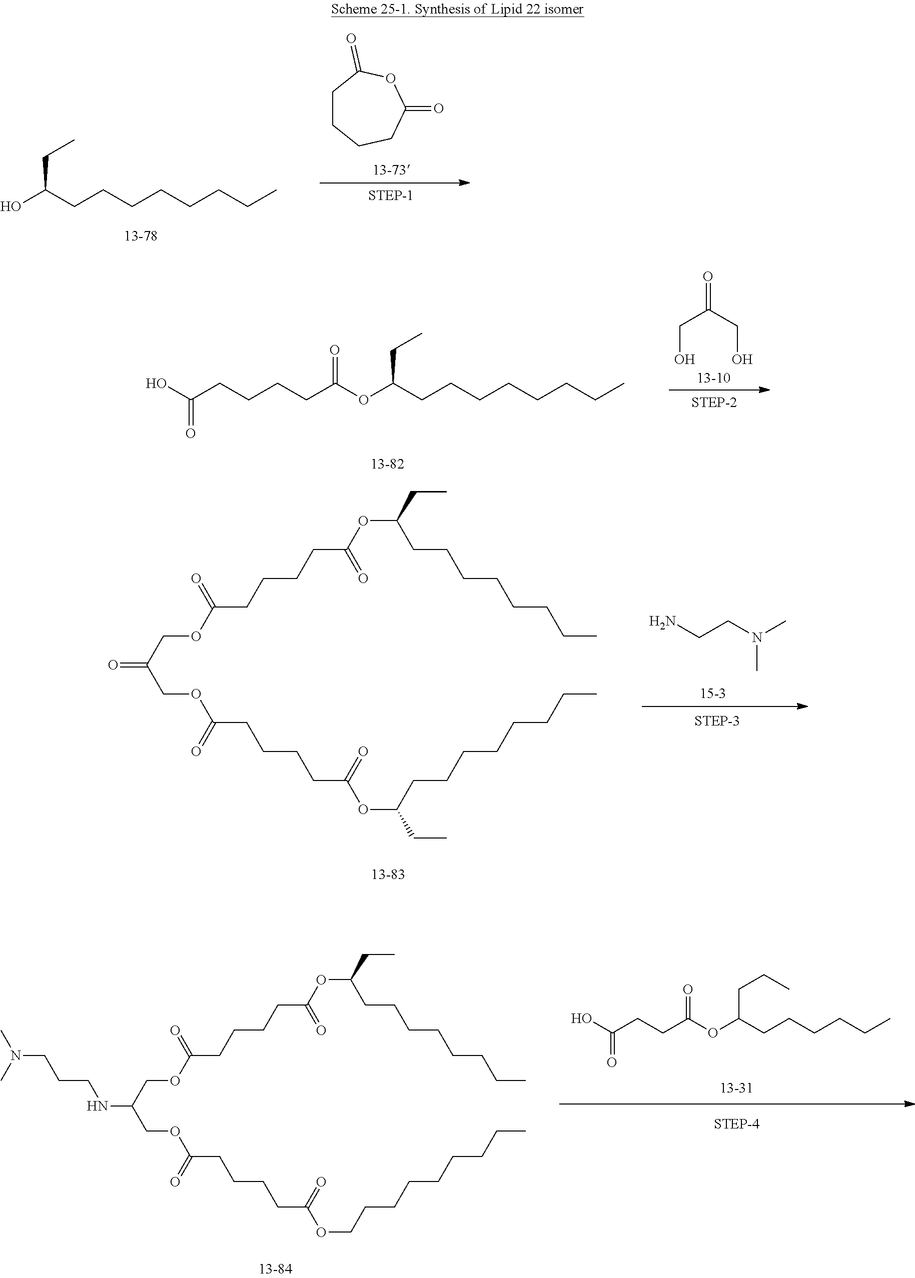

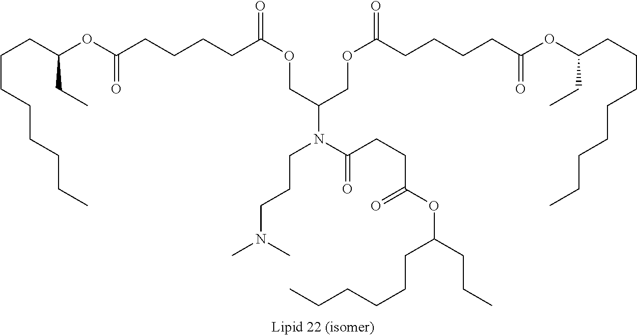 [SMILES: CCCCCCCC[C@H](CC)OC(=O)CCCCC(=O)OCC(COC(=O)CCCCC(=O)O[C@@H](CC)CCCCCCCC)N(CCCN(C)C)C(=O)CCC(=O)OC(CCC)CCCCCC]

Lipid 22 as a racemic mixture was synthesized as provided in scheme 25-2 below. Lipid 22 was accessed using methods described for Lipid 22 isomer above by replacing alcohol isomer 13-78 with racemic alcohol 13-78 rac.

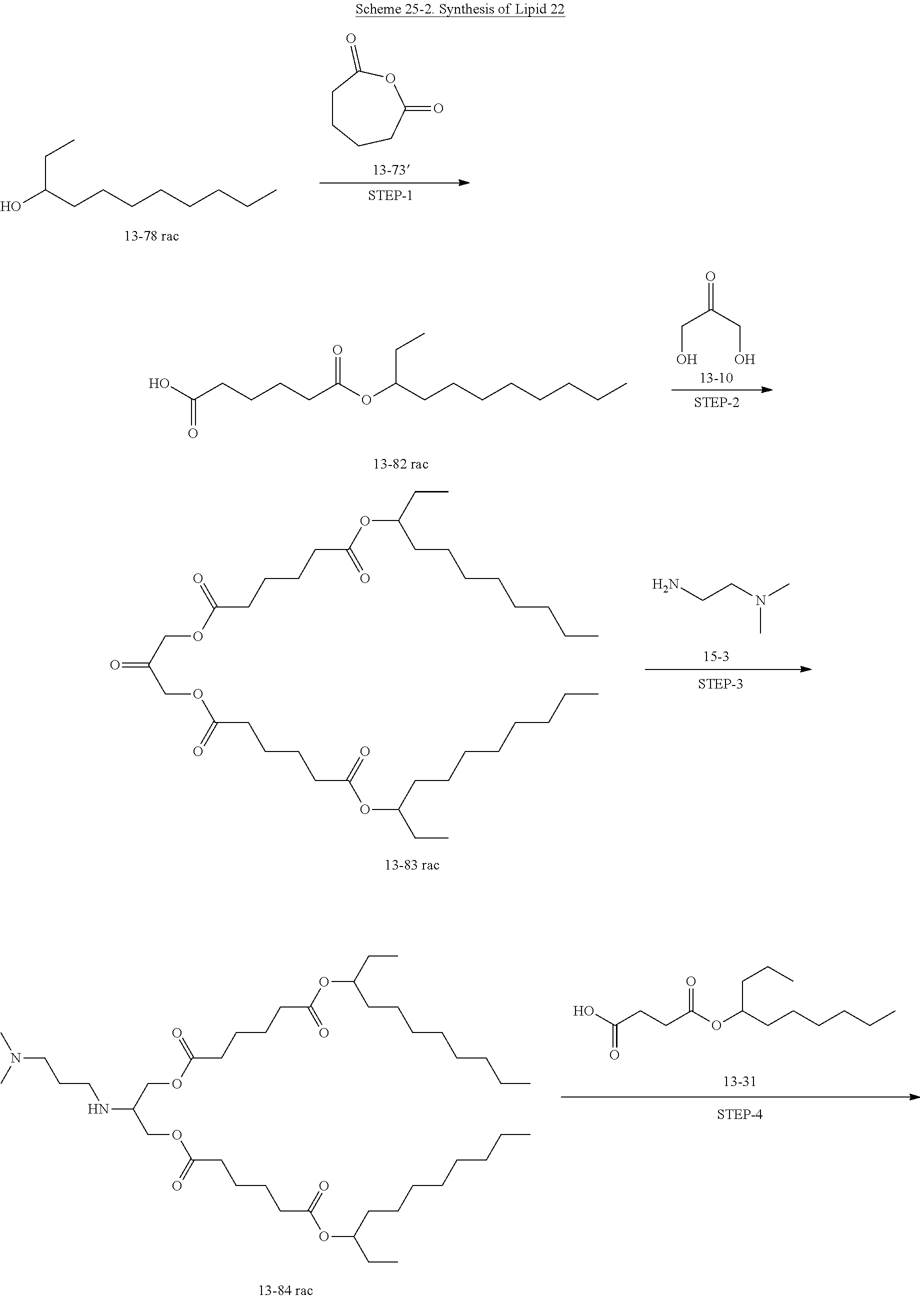

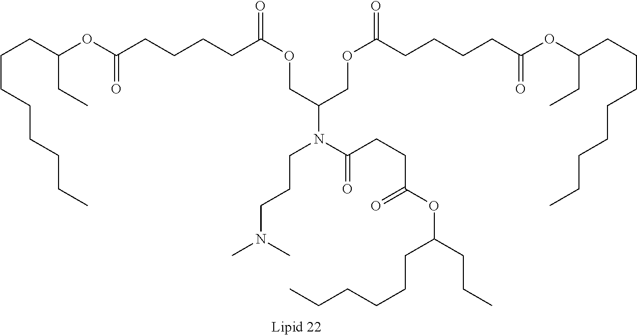 [SMILES: CCCCCCCCC(CC)OC(=O)CCCCC(=O)OCC(COC(=O)CCCCC(=O)OC(CC)CCCCCCCC)N(CCCN(C)C)C(=O)CCC(=O)OC(CCC)CCCCCC]

Alternatively, starting material 3-undecanol, 13-78-racemic (31.6 mmol, 5 g, 1 eq.) was acylated with adipic acid, 13-82 (9.8 mmol g, 1.68 g, 2.0 eq.) using EDCI (2.26 g, 1.2 eq, 11.8 mmol), DMAP (143 mg, 0.12 eq, 1.2 mmol) and TEA (6.8 mL, 5.0 eq, 49.0 mmol) in a mixture of 20 mL of DCM and 20 mL DMF at room temperature, overnight to obtain 1.35 g (46%) of acid intermediate, 13-83 rac. Starting material, 1,3-dihydroxyacetone, 13-10 (2.05 mmol, 184 mg, 1.0 eq.) was diacylated with acid Intermediate 13-83-rac (1.35 g, 2.2 eq, 4.5 mmol) using EDCI (856 mg, 2.2 eq, 4.5 mmol), DIPEA (782 μL, 2.2 eq, 4.5 mmol), and DMAP (127 mg, 0.5 eq, 1.03 mmol) in 6 mL of DCM at room temperature overnight to obtain 610 mg (46%) of ketone intermediate 13-84-rac.

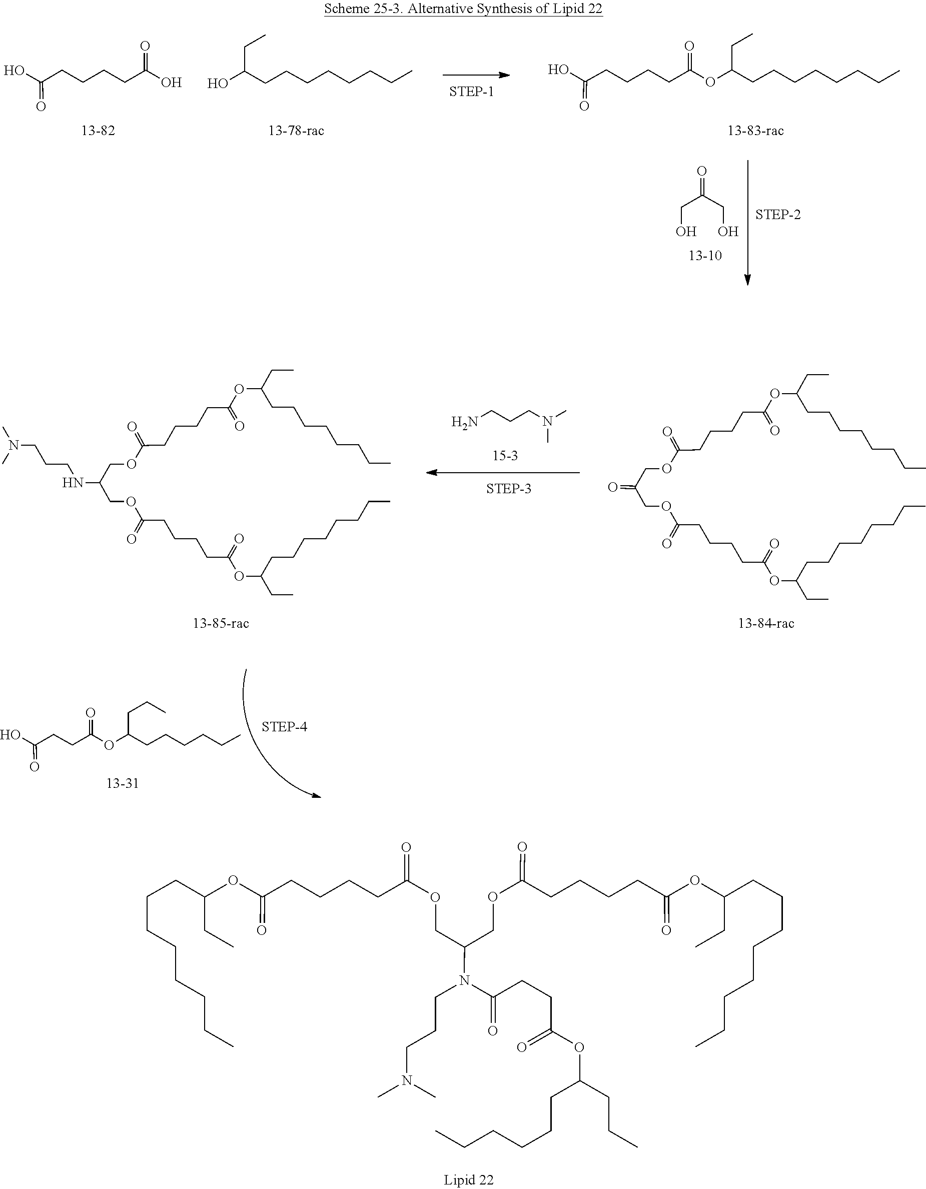

Intermediate 13-84-rac (0.93 mmol, 610 mg, 1.0 eq.) was converted to diamine intermediate 13-85-rac by reductive amination with N,N-dimethylamino-3-aminopropane, 15-3 (1.86 mmol, 233 μL, 2.0 eq.) using Na(OAc)₃BH (1.2 mmol, 249 mg, 1.2 eq.), acetic acid (1.86 mmol, 107 μL, 2.0 eq.) in 3 mL of DCM at room temperature for 3 hours. Purification of crude product by silica column chromatography (10% MeOH in DCM) yielded 210 mg of purified Intermediate 13-85-rac. Acid chloride 13-31′ (0.85 mmol, 233 mg, 3.0 eq.) from a previous batch was used for N-acylation of diamine intermediate 13-85-rac (0.28 mmol, 210 mg, 1 eq.) using TEA (1.4 mmol, 197 μL, 5.0 eq.) in 3 mL of Toluene at room temperature overnight to yield Lipid 22. Crude product was purified 2× by column chromatography (DCM:10% MeOH in DCM) to obtain 56 mg (60%) of pure Lipid 22 (>99% by HPLC-CAD) and characterized by proton NMR and mass spectrometry (FIG. 4Z-1 and FIG. 4Z-2 for proton NMR and HPLC-CAD and Table 5 for Mass Spectrometry data A,B,C).

### Synthesis of Lipid 23

Lipid 23 was synthesized as provided in scheme 26 below. Briefly, O-acylation of dihydroxyacetone with acid 13-31 using conditions described in Lipid 9 synthesis yielded ketone 13-70. Reductive amination of 13-70 with amine 15-3 using conditions described in the Lipid 9 synthesis yielded intermediate 13-71. Subsequent N-acylation of intermediate 13-71 with acid 13-31 using conditions analogous to those used for Lipid 9 synthesis afforded Lipid 23.

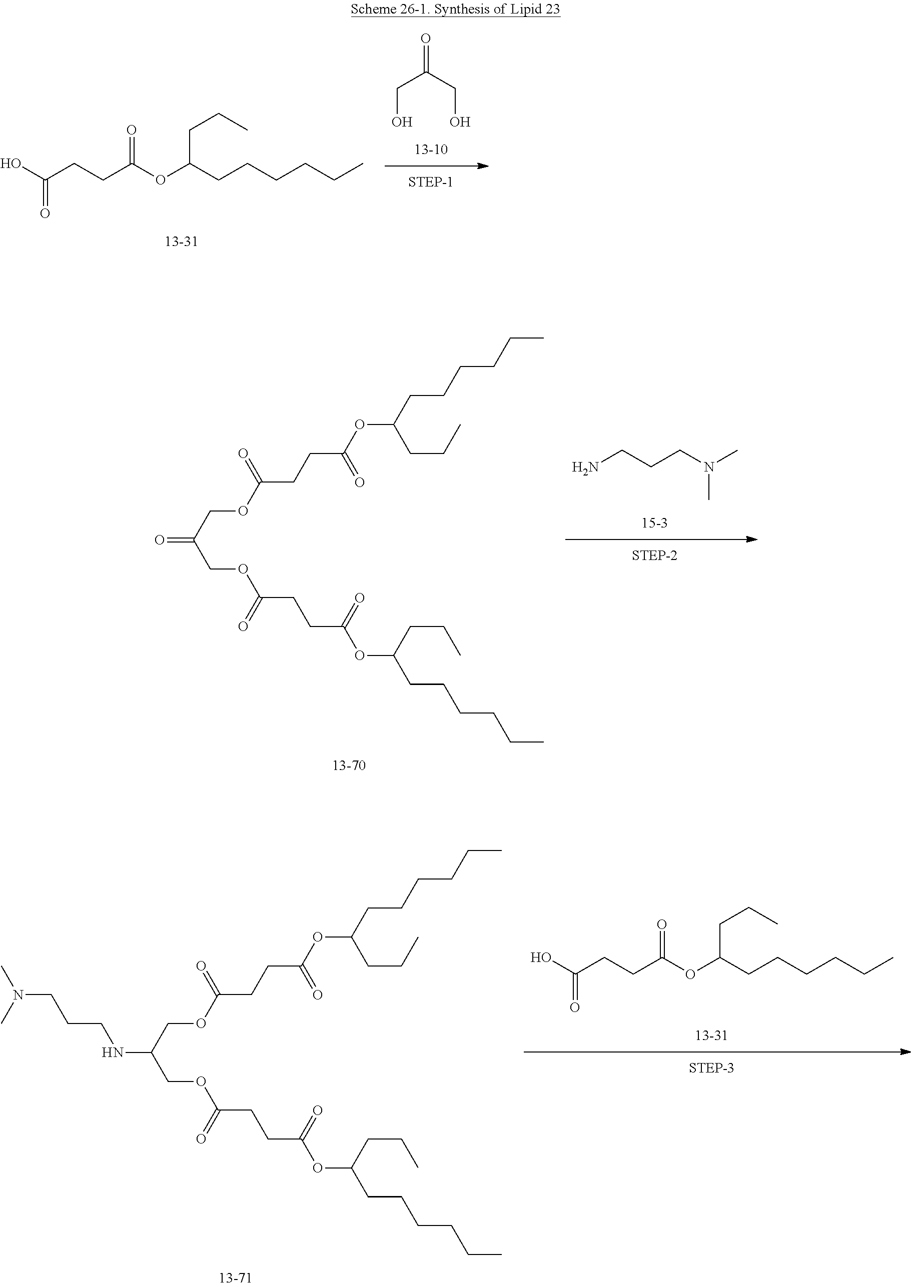

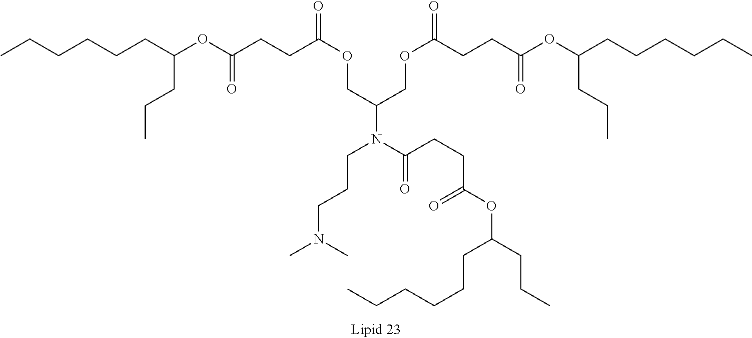 [SMILES: CCCCCCC(CCC)OC(=O)CCC(=O)OCC(COC(=O)CCC(=O)OC(CCC)CCCCCC)N(CCCN(C)C)C(=O)CCC(=O)OC(CCC)CCCCCC]

### Synthesis of Lipid 23A

Starting material 3-undecanol, 13-78-racemic (14.4 mmol, 2.47 g, 1 eq.) was acylated with suberic acid, 13-77 (5.0 g, 2.0 eq, 82.7 mmol) using EDCI (3.3 g, 1.2 eq, 17.3 mmol), DMAP (211 mg, 0.12 eq, 1.73 mmol) and TEA (10.0 mL, 5.0 eq, 72.0 mmol) in a mixture of 20 mL of DCM and 20 mL DMF at room temperature overnight to obtain 2 g (43%) of acid intermediate, 13-879-rac. Starting material, 1,3-dihydroxyacetone, 13-10 (2.8 mmol, 250 mg, 1.0 eq.) was diacylated with acid Intermediate 13-79-rac (2.0 g, 2.2 eq, 6.1 mmol) using EDCI (1.16 g, 2.2 eq, 6.1 mmol), DIPEA (1.06 mL, 2.2 eq, 6.1 mmol), and DMAP (172 mg, 0.5 eq, 1.4 mmol) in 8 mL of DCM at room temperature overnight to obtain 690 mg (35%) of ketone intermediate 13-80-rac.

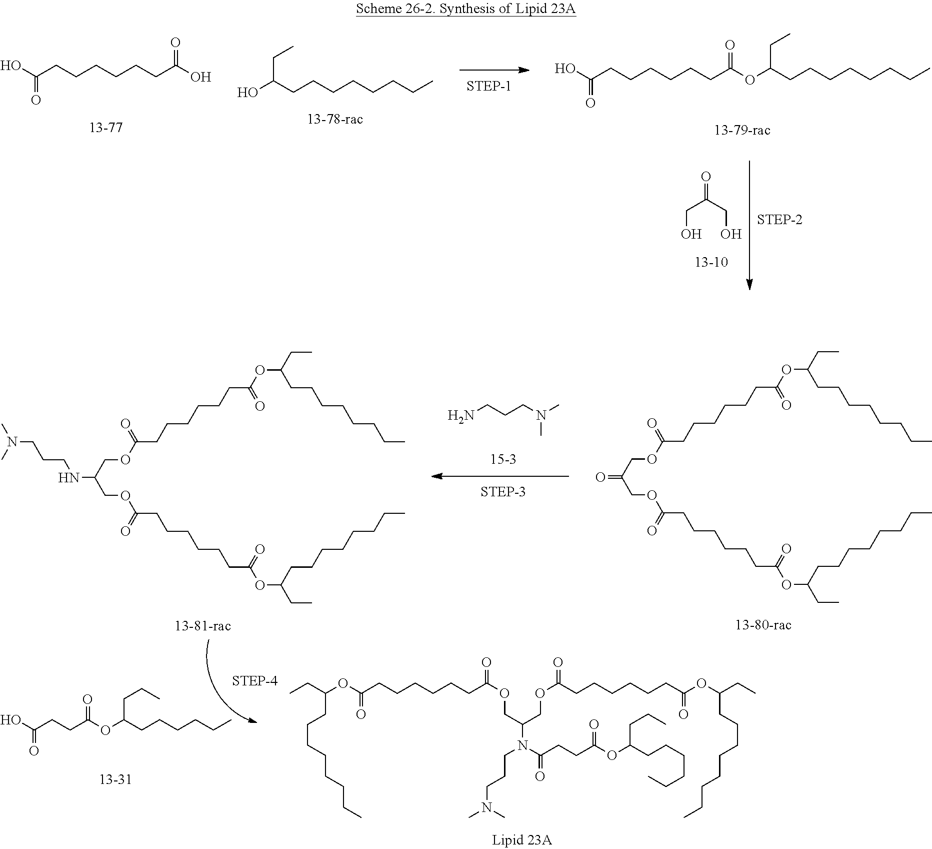

Intermediate 13-80-rac (0.97 mmol, 690 mg, 1.0 eq.) was converted to diamine intermediate 13-81-rac by reductive amination with N,N-dimethylamino-3-aminopropane, 15-3 (1.94 mmol, 243 μL, 2.0 eq.) using Na(OAc)₃BH (1.2 mmol, 249 mg, 1.2 eq.), acetic acid (1.94 mmol, 112 μL, 2.0 eq.) in 3 mL of DCM at room temperature for 3 hours. Purification of crude product by silica column chromatography (10% MeOH in DCM) yielded 520 mg of purified Intermediate 13-81-rac. Acid chloride 13-31′ (1.63 mmol, 447 mg, 2.5 eq.) from a previous batch was used for N-acylation of diamine intermediate 13-81-rac (0.65 mmol, 520 mg, 1 eq.) using TEA (3.25 mmol, 458 μL, 5.0 eq.) in 4 mL of toluene at room temperature overnight to yield Lipid 23A. Crude product was purified 2× by column chromatography (DCM:10% MeOH in DCM) to obtain 230 mg (310%) of pure Lipid 23A (>99% by HPLC-UV and 96% by HPLC-CAD) and characterized by proton NMR and mass spectrometry (FIG. 4AA-1 and FIG. 4AA-2 for proton NMR and HPLC-CAD and Table 5 for Mass Spectrometry data).

### Synthesis of Lipid 24

Lipid 24 was synthesized as provided in scheme 27 below. Briefly, acid 13-34 was accessed by O-acylation of mono-protected di-acid 13-72 with alcohol 13-29 and subsequent deprotection of intermediate 13-73 to yield acid, 13-74. O-acylation of dihydroxyacetone with intermediate 13-74 using conditions described in Lipid 17 synthesis yielded ketone 13-75. Reductive amination of 13-75 with amine 15-3 using conditions described in the Lipid 17 synthesis yielded intermediate 13-76. Subsequent N-acylation of intermediate 13-76 with acid 13-31 using conditions analogous to those used for Lipid 9 synthesis afforded Lipid 24.

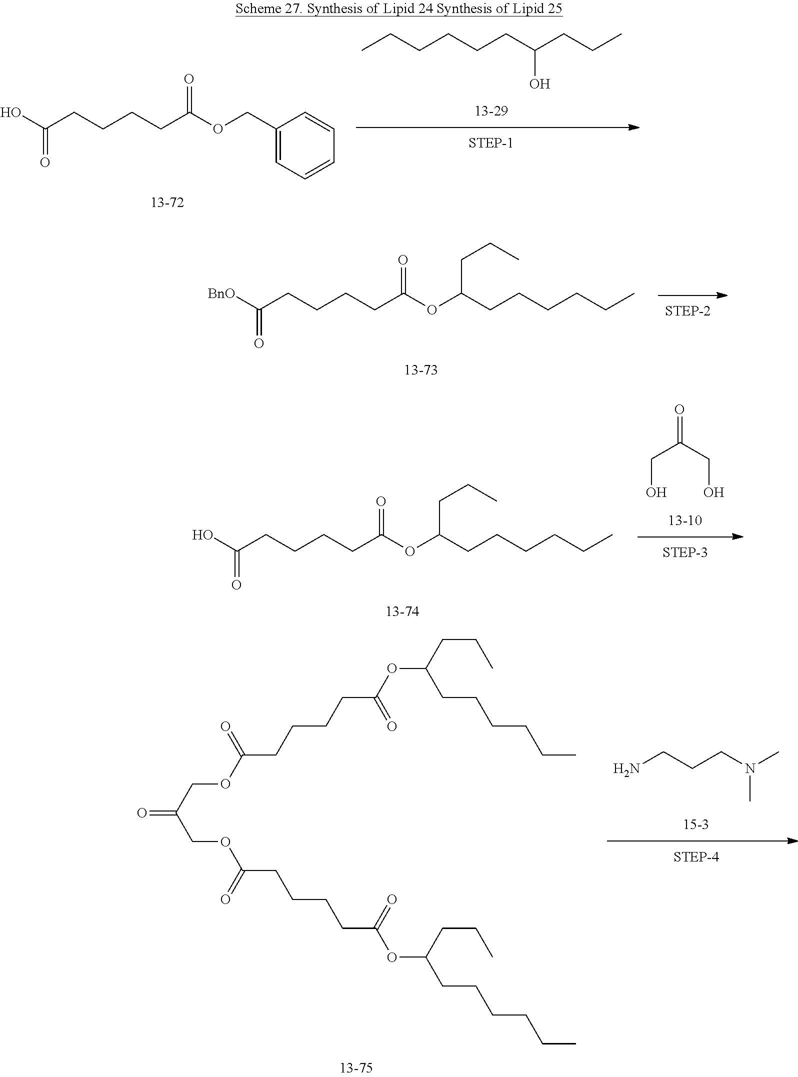

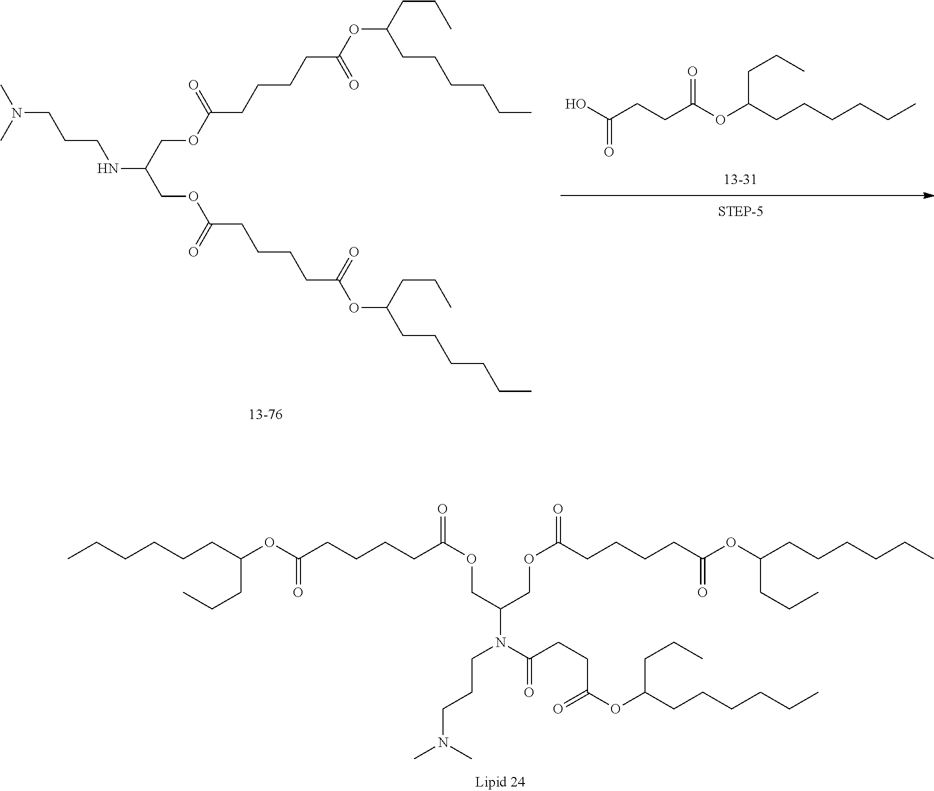 [SMILES: CCCCCCC(CCC)OC(=O)CCCCC(=O)OCC(COC(=O)CCCCC(=O)OC(CCC)CCCCCC)NCCCN(C)C ; CCCCCCC(CCC)OC(=O)CCC(=O)O ; CCCCCCC(CCC)OC(=O)CCCCC(=O)OCC(COC(=O)CCCCC(=O)OC(CCC)CCCCCC)N(CCCN(C)C)C(=O)CCC(=O)OC(CCC)CCCCCC]

Lipid 25 was synthesized as provided in scheme 28 below. Briefly, ring opening addition of alcohol 13-29 to cyclic anhydride 13-52′ yielded acid intermediate 13-85. O-acylation of dihydroxyacetone with intermediate 13-85 using conditions described in Lipid 17 synthesis yielded ketone 13-86. Reductive amination of 13-86 with amine 15-3 using conditions described in the Lipid 17 synthesis yielded intermediate 13-87. Subsequent N-acylation of intermediate 13-87 with acid 13-31 using conditions analogous to those used for Lipid 9 synthesis afforded Lipid 25.

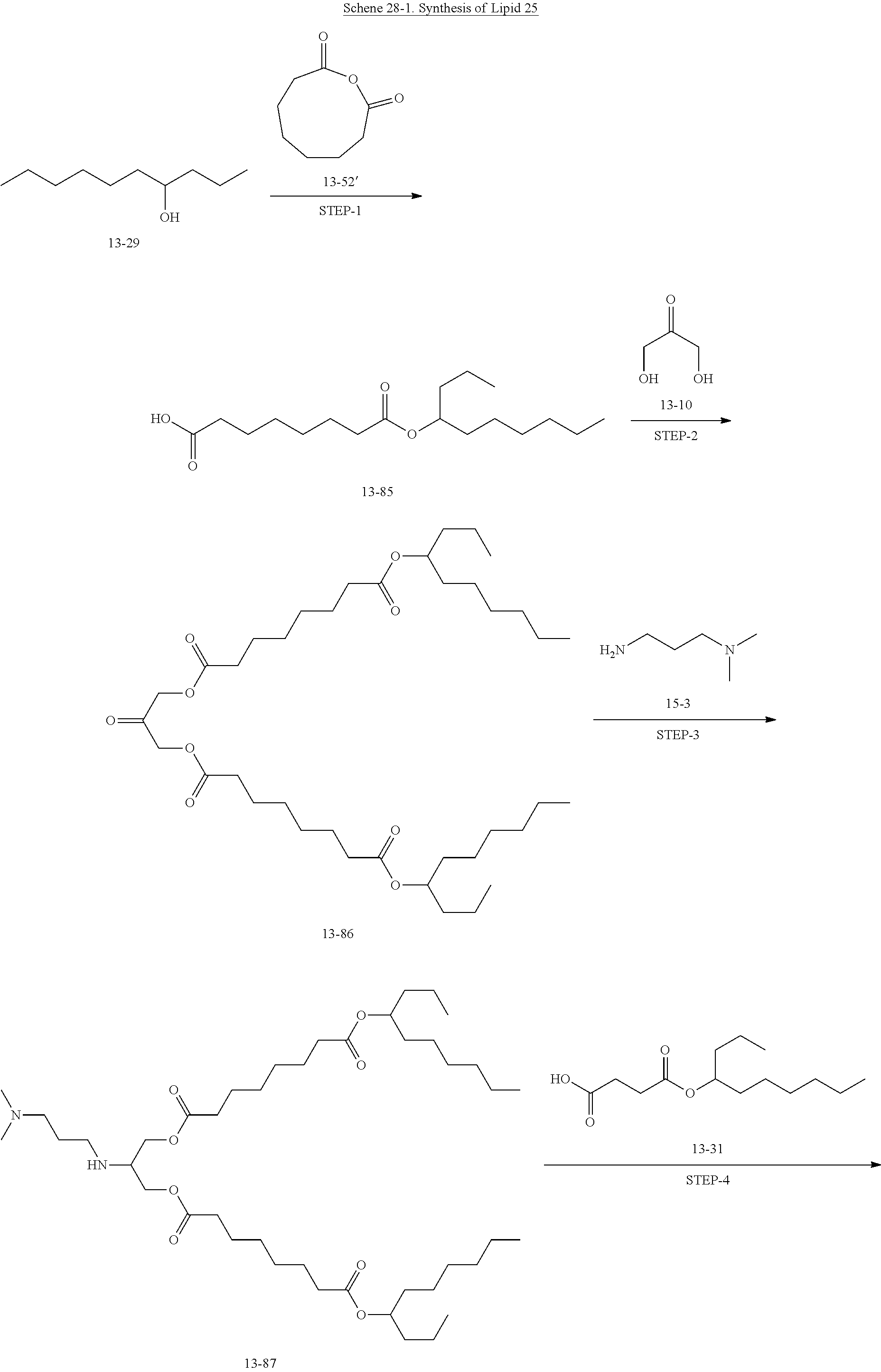

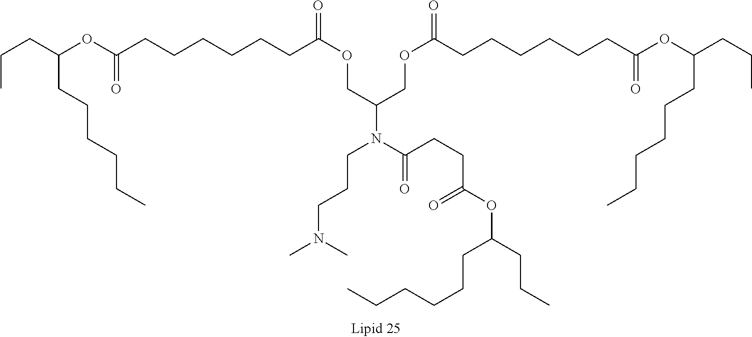 [SMILES: CCCCCCC(CCC)OC(=O)CCCCCCC(=O)OCC(COC(=O)CCCCCCC(=O)OC(CCC)CCCCCC)N(CCCN(C)C)C(=O)CCC(=O)OC(CCC)CCCCCC]

### Synthesis of Lipid 25A

Starting material benzyl protected glycolic acid 14-24 (15.4 mmol, 2.61 g, 1 eq.) was acylated with 2-hexyldecanoic acid 14-25 (6.10 g, 96%, 1.48 eq, 22.84 mmol) using EDCI (5.80 g, 1.97 eq, 30.26 mmol), DMAP (0.40 g, 0.21 eq, 3.27 mmol) and DIPEA (5.4 mL g, 1.97 eq, 30.33 mmol) in 60 mL of dichloromethane at room temperature overnight yielding 2.75 g (49%) of protected intermediate ester 14-26. Intermediate 14-26 (10.3 mmol, 4.1 g, 1.0 eq.) was deprotected using Pd/C (930 mg, 32% w/w), EtOAc (35 mL) at room temperature overnight yielding 1.72 g (82%) of acid intermediate 14-27.

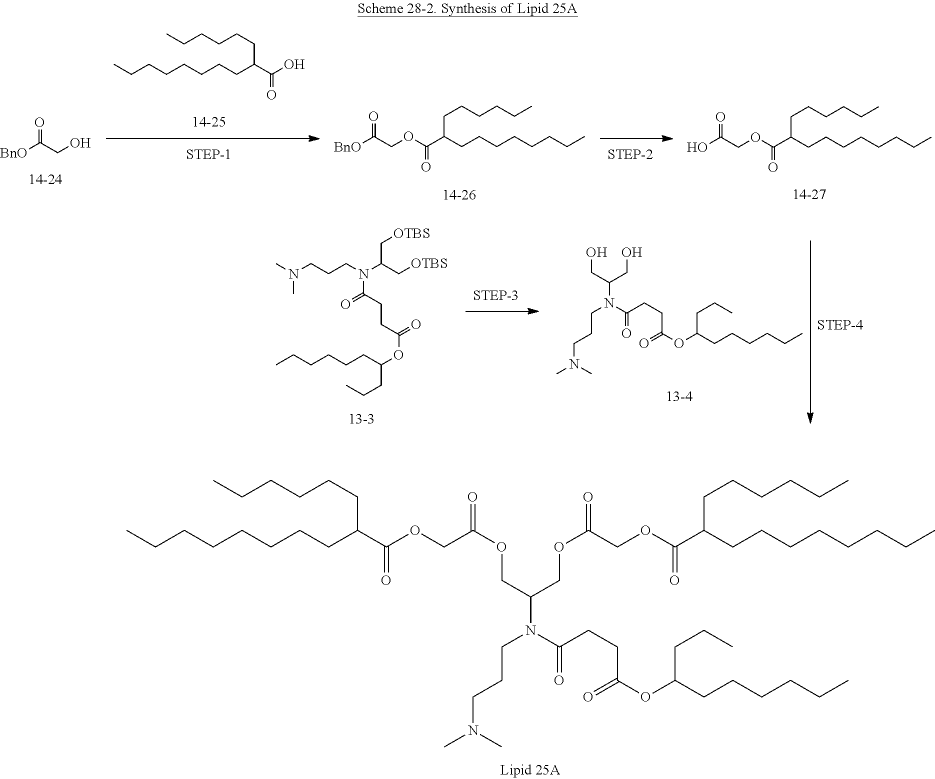

Protected intermediate 13-3 (600 mg, 0.9 mmol, 1 eq.) was treated with Hydrogen Fluoride/Pyridine (0.92 g, 10.0 eq, 9.3 mmol) in 4.0 mL THF at room temperature for 2 hours yielding dihydroxyl intermediate 13-4. Deprotection was confirmed by TLC and mass spectrometry. Acid intermediate 14-27 (5.4 mmol, 1.72 g, 1 eq.) was converted to the corresponding acid chloride 14-27′ using oxalyl chloride (2.36 g, 3.4 eq, 18.59 mmol) and DMF (100 μL, catalytic quantity) in 5.0 mL of Toluene at room temperature for 2 hours and conversion to chloride intermediate was confirmed by TLC.

Crude di-hydroxy intermediate 13-4 (350 mg, 0.84 mmol, 1 eq.) and crude acid chloride 14-27′ (1.58 g, 6.0 eq, 5.04 mmol) were combined with TEA (0.85 g, 10.0 eq, 8.4 mmol) in 9 mL of toluene at room temperature, overnight. Crude product was purified on ISCO column chromatography on silica column eluting with DCM and 10% MeOH in DCM yielding 240 mg (85%) of 99% pure (HPLC-CAD) Lipid 25A. (see FIG. 4AC-1 and FIG. 4AC-2 for characterization by proton NMR and LC-CAD purity and Table 5 for Mass Spectrometry data).

### Synthesis of Lipid 27

Lipid 27 was synthesized as provided in scheme 29 below and as follows. Ring opening of starting material caprolactone 14-30 (2 g, 17.5 mmol) using 5 mL of 0.5 M NaOH at room temperature for 2 hours yielded 6-hydroxyhexanoic acid 14-31 (1.8 g, 78%). Additional 14-31 was produced in a second 2 g scale caprolactone hydrolysis reaction using identical conditions to obtain 1.9 g (82%) of 14-31. 6-hydroxyhexanoic acid 14-31 (1 g, 7.5 mmol) was benzylprotected using DBU (1.38 g, 1.2 eq, 9 mmol), Benzylbromide (1.68 g, 1.3 eq, 9.8 mmol) in 6 mL of MeOH and 10 mL of DMF at 0° C. to room temperature, overnight yielding protected intermediate 14-32 (1.3 g, 77%). Additional 14-31 (2 g, 15.1 mmol) was protected using DBU (2.76 g, 1.2 eq, 18.1 mmol), Benzylbromide (3.36 g, 1.3 eq, 19.6 mmol) in 12 mL of MeOH and 20 mL of DMF at 0° C. to room temperature, overnight yielding protected intermediate 14-32 (2.65 g, 79%).

Intermediate 14-32 (2.45 g, 1 eq, 11 mmol) was used to acylate acid 14-25 (2.59 g, 1.5 eq, 10 mmol) using EDCI (2.58 g, 2 eq, 13.4 mmol), DIPEA (1.74 g, 2 eq, 13.4 mmol) and DMAP (0.16 g, 0.2 eq, 1.3 mmol) in 20.0 mL DCM at room temperature overnight. Crude product was purified by column chromatography (1×) yielding 4.8 g (94%) of pure protected intermediate 14-33. Subsequent deprotection of 14-33 (4.8 g, 22 mmol) by hydrogenation on Pd/C/Hydrogen gas (0.6 g, 20% w/w) in 30 mL of ethylacetate yielded 3.68 g (95%) of free acid 14-34 of column purified material.

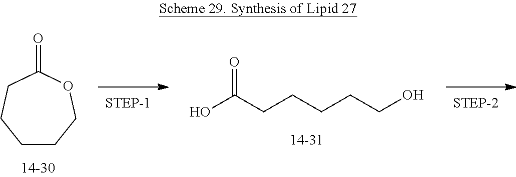 [SMILES: O=C1CCCCCO1 ; O=C(O)CCCCCO]

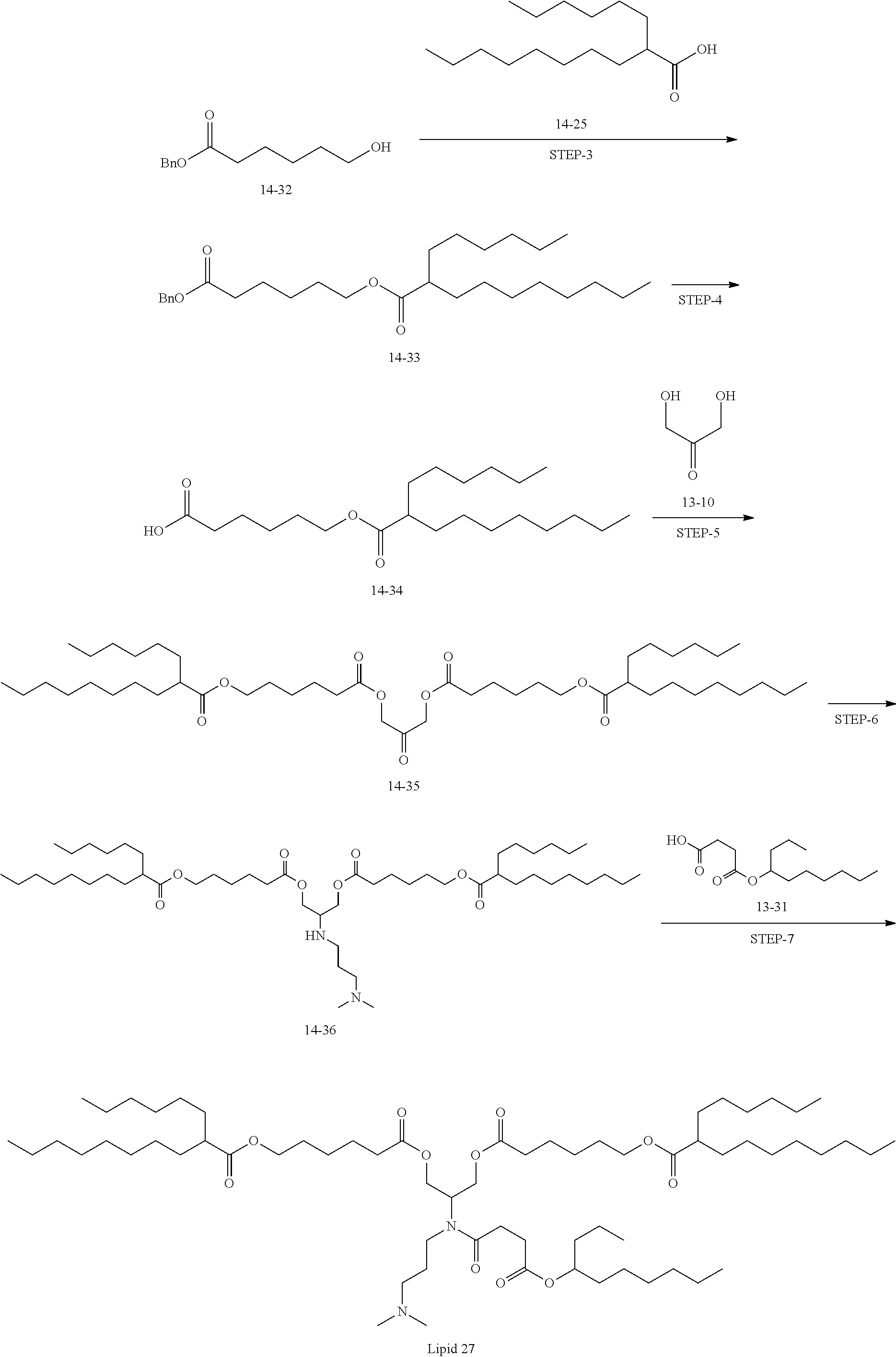

Acid intermediate 14-34 (1.74 g, 2.5 eq, 5.5 mmol) was used to acylate dihydroxyacetone, 13-10 (200 mg, 1.0 eq, 6 mmol), EDCI (1.06 g, 2.5 eq, 5.5 mmol), DIPEA (0.71 g, 2.5 eq, 5.5 mmol), and DMAP (54 mg, 0.2 eq, 0.4 mmol) in 10.0 mL DCM at room temperature, overnight to obtain ketone 14-35. Crude product was purified by column chromatography to obtain 1.27 mg (76%) of pure 14-35. Reductive amination of 14-35 (1.26 mg, 1.0 eq, 1.5 mmol) with amine 15-3 (0.32 g, 2.0 eq, 3.1 mmol) using acetic acid (0.19 g, 2.0 eq, 3.1 mmol) and Na(OAc)₃BH (0.50 g, 1.5 eq, 2.3 mmol) in 15 mL DCM at room temperature of 3 hours yielded intermediate 14-36 (330 mg, 34%).

Compound 13-31 (290 mg, 1.1 mmol) from a previous batch was converted to the corresponding acid chloride 13-31′ using oxalyl chloride (0.48 g, 3.4 eq, 3.8 mmol) and DMF (20 μL, catalytic quantity) in 4 mL of Toluene for 2 hours at room temperature. Crude 13-31′ (0.29 g, 3.0 eq, 1.1 mmol) was used for N-acylation of 14-36 (330 mg, 1.0 eq, 0.3 mmol) using TEA (0.26 mL, 5.0 eq, 1.8 mmol) 5 mL toluene at room temperature, overnight to obtain column purified Lipid 20 (180 mg, 43%) of >98% purity (HPLC-CAD).

See FIG. 4AE-1 and FIG. 4AE-2 for Lipid 27 NMR spectrum, and reverse phase LC-CAD chromatogram, and Table 5 for product mass.

### Synthesis of Lipid 28

Lipid 28 was synthesized as provided in scheme 30 below and as follows. Intermediate 14-36 was produced as described above (see Synthesis of Lipid 27).

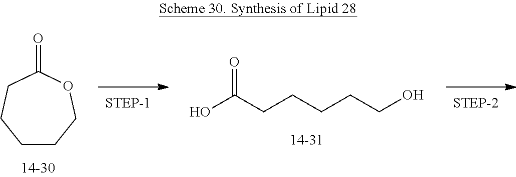 [SMILES: O=C1CCCCCO1 ; O=C(O)CCCCCO]

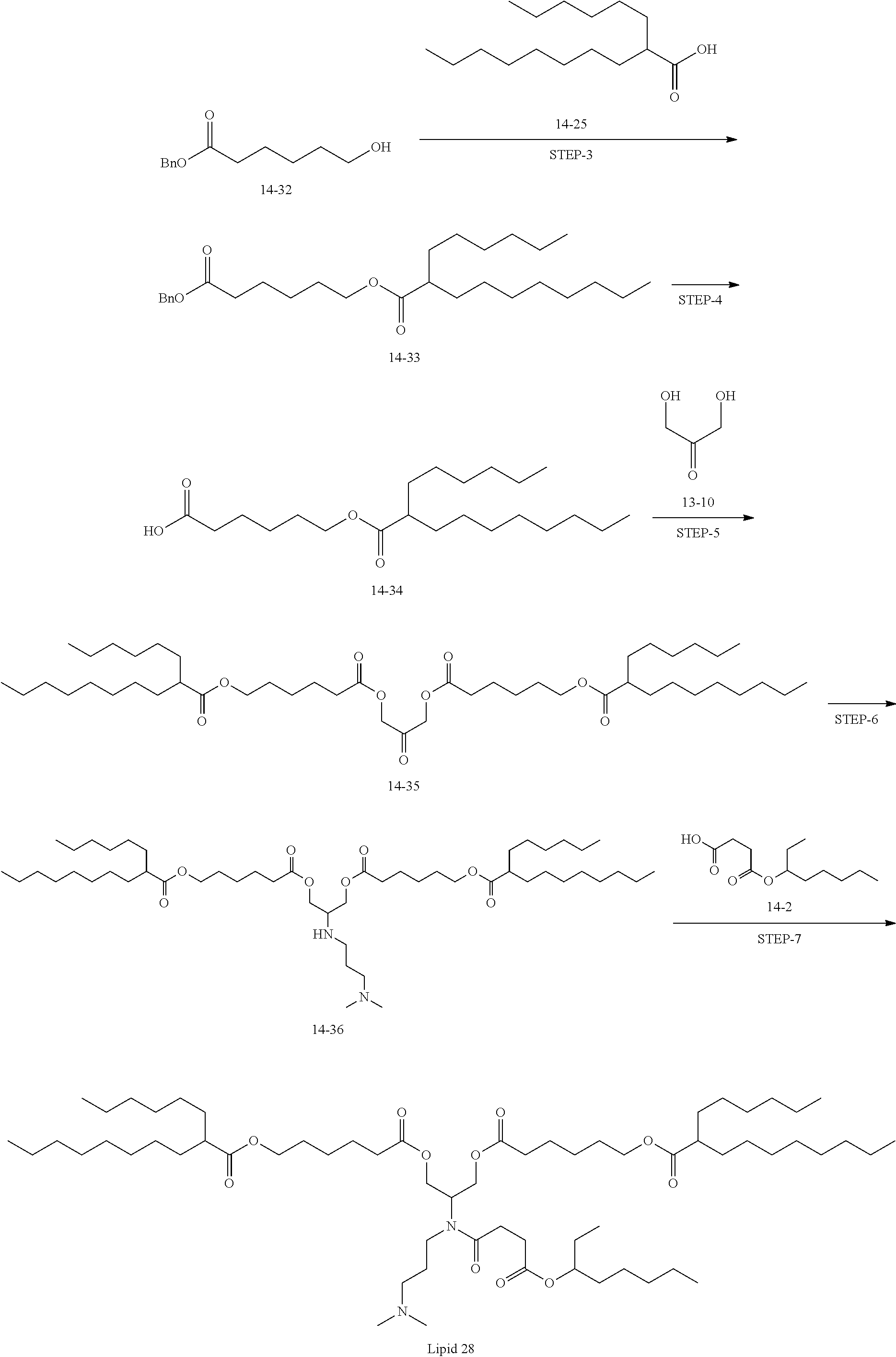

Acid intermediate 14-2 was produced by acylation of starting material 3-hydroxyoctanol 14-1 with succinic acid using 13-30 (1.53 g, 2.0 eq, 15.3 mmol) and DMAP (0.93 g, 1.0 eq, 7.6 mmol) and 2 mL of Pyridine in a mixture of 2 mL of THF and 5 mL of DCM to obtain 1.1 g (64%) of 14-2. 14-2 (348 mg, 1.51 mmol) was converted to the corresponding acid chloride 14-2″ using oxalyl chloride (0.651 g, 5.13 mmol, 3.4 eq) and DMF (40 μL, catalytic quantity) in 3 mL of Toluene for 2 hours at room temperature. Crude 14-2′ (0.348 g, 1.51 mmol, 3.1 eq.) was used for N-acylation of 14-36 (430 mg, 1.51) using TEA (0.41 mL, 2.94 mmol, 6.02 eq) in 6 mL toluene and 2.5 mL DCM at room temperature, overnight to obtain column purified (eluting with 10% methanol in DCM) Lipid 20 (148 mg, 28%) of 85% purity (HPLC-CAD). Second column purification (eluting with 5% methanol in DCM) yielded 115 mg of 94% purity (HPLC-CAD).

See FIG. 4AF-1 for Lipid 27 NMR spectrum, FIG. 4AF-2 for Lipid 28 reverse phase LC-CAD chromatogram, and Table 5 for product mass.

### Synthesis of Lipid 29

Benzyl protected malic acid 14-4 (14.1 mmol, 3.18 g, 1 eq.) was acylated with N-decanoic acid 14-12 (3.86 g, 1.5 eq, 21.2 mmol) using HATU (8.1 g, 1.5 eq, 21.2 mmol), DBU (4.3 g, 2.0 eq, 28.3 mmol) in 30 mL of DMF at room temperature overnight yielding 1.9 g (36%) of protected intermediate ester 14-13. Intermediate 14-13 (5.2 mmol, 1.9 g, 1 eq.) was acylated with hexanoyl chloride 14-6, (2.8 g, 4.0 eq, 20.8 mmol), TEA (2.63 g, 5.0 eq, 26.0 mmol), DMAP (127 mg, 0.2 eq, 1.0 mmol) in 20 mL of Toluene at room temperature, overnight to obtain intermediate 14-14 (630 mg, 26%). Intermediate 14-14 (2.8 mmol, 1.3 g, 1.0 eq.) was deprotected using Pd/C (260 mg, 32% w/w) in 15 mL Ethyl Acetate at room temperature overnight yielding 1.025 g (98%) of acid intermediate 14-15.

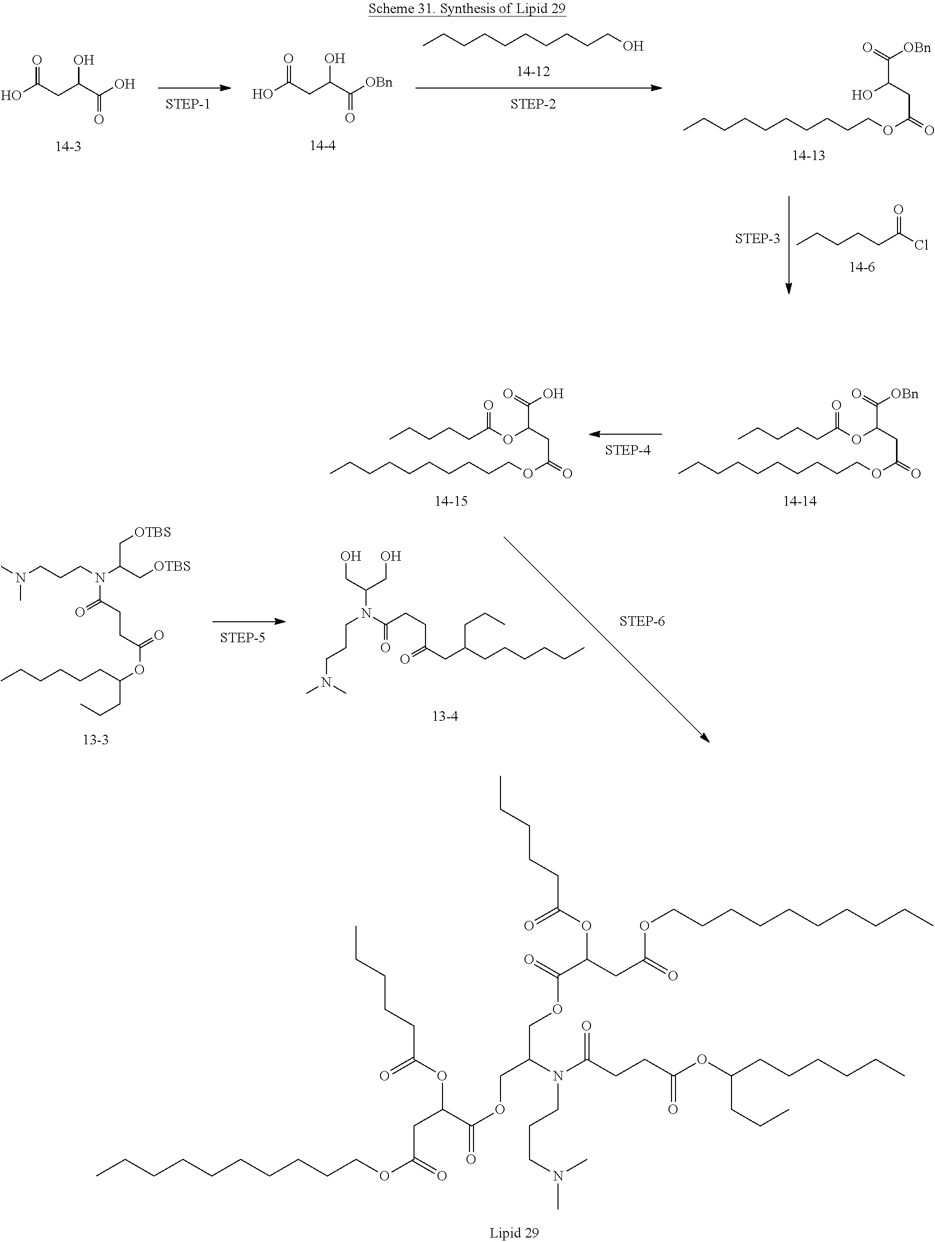

Acid intermediate 14-15 (2.6 mmol, 1.0 g, 1 eq.) was converted to the corresponding acid chloride 14-15′ using oxalyl chloride (0.82 mL, 3.4 eq, 9.1) and DMF (100 L, catalytic quantity) in 6.0 mL of Toluene at room temperature for 2 hours and conversion to chloride intermediate was confirmed by TLC.

Protected intermediate 13-3 (0.72 mmol, 300 mg) using HF-pyridine, (0.71 mL, 10.0 eq, 7.2 mmol) in 4.0 mL THE at room temperature, overnight and crude di-hydroxy intermediate 13-4 (195 mg, 0.46 mmol, 1 eq.) and crude acid chloride 14-15′ (1.097 g, 6.0 eq, 2.7 mmol) were combined with TEA (0.64 mL, 10.0 eq, 4.6 mmol) in 5 mL of toluene at room temperature, overnight. Crude product was purified on ISCO column chromatography on silica column eluting with DCM and 10% MeOH in DCM yielding to obtain 270 mg (51%) of >99% pure (HPLC-CAD) Lipid 29. (see FIG. 4AG-1 and FIG. 4AG-2 for characterization by proton NMR and LC-CAD purity, and Table 5 for Mass Spectrometry data).

### Synthesis of Lipid 31

Lipid 31 was synthesized as provided in scheme 32 below and as follows. Starting material 15-1 (68 mmol, 10 g, 1 eq.) was treated with p-toluene sulfonyl chloride (70 mmol, 13.3 g, 1.03 eq.) using Pyridine (80 mmol, 10.1 ml, 1.2 eq.) in 150 mL DCM to obtain protected intermediate 15-2. Crude product was recrystallized in ethyl acetate and hexane yielding 20.4 g (99%) of pure intermediate 15.2. Intermediate 15-4 was accessed by reaction of 15-2 (16.5 mmol, 5 g, 1.2 eq.) and diamine 15-3 (33 mmol, 3.35 g, 2 eq.) in 40 mL dioxane under reflux conditions. Crude product was purified by column chromatography to obtain 3.5 g (91%) of pure intermediate 15-4. N-acylation of 15-4 (108 mg, 0.268 mmol) using nonanoic acid 13-12 (0.67 mmol, 106 mg, 2.5 eq) using EDCI (0.67 mmol, 128 mg, 2.5 eq.) and DIEA (0.67 mmol, 86 mg, 2.5 eq) and DMAP (3 mg) in 10 mL DCM yielded amine 15-5. Crude product was purified by column chromatography to obtain 113 mg (65%) of purified diamine 15-5. Diol intermediate 15-6 was accessed by deprotection of 15-5 (113 mg) in 4 mL of 1M HCl and THE (1:3 v/v) at room temperature for 8 hours in quantitative yield (102 mg). Intermediate 15-6 (0.3 mmol, 100 mg, 1 eq) was acylated with linoleic acid 1-5 (0.9 mmol, 250 mg, 3 eq) using EDCI (0.9 mmol, 172 mg, 3 eq), DIPEA (0.9 mmol, 116 mg) and DMAP (10 mg, catalytic quantity) to obtain Lipid 31. Crude product was purified by column chromatography yielding 120 mg (46%) of pure Lipid 31 (>99% purity by LC-ELSD) and characterized by proton NMR and Mass Spectrometry (see FIG. 4R-1 for Lipid 31 NMR spectrum, FIG. 4R-2 for Lipid 31 LC-MS, and Table 5 for product mass).

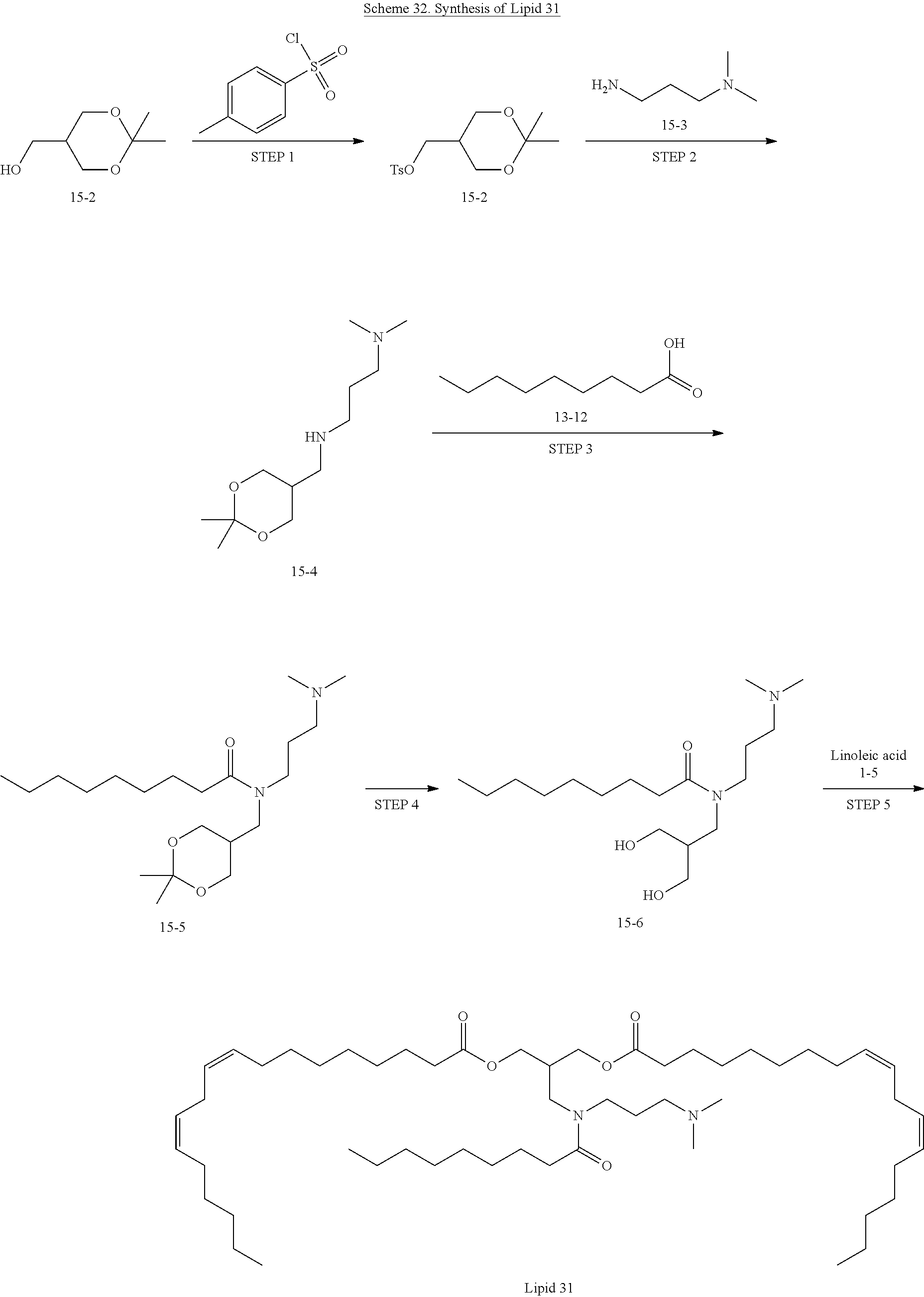

### Synthesis of Lipid 32

Lipid 32 was synthesized as provided in scheme 33 below and as follows. Intermediate 15-4 was produced as described for Lipid 31 above (steps 1 and 2, Scheme 30). N-acylation of 15-4 (4.34 mmol, 1 g, 1.0 eq) using 2-ethyl heptanoic acid 13-13 (10.85 mmol, 1.71 g, 2.5 eq) using EDCI (10.85 mmol, 2.07 g, 2.5 eq), DIEA (10.85 mmol, 1.40 g, 2.5 eq) and DMAP (10 mg) in 100 mL DCM yielded amine 15-7. Crude product was purified by column chromatography to obtain 724 mg (52%) of purified diamine 15-7. Diol intermediate 15-8 was accessed by deprotection of 15-7 (714 mg) in 3 mL of 1M HCl and 7 mL THE at room temperature for 1 hour in quantitative yield. Intermediate 15-8 (1.9 mmol, 630 mg, 1 eq) was acylated with linoleic acid 1-5 (6.49 mmol, 1.82 g, 3.4 eq) using EDCI (6.49 mmol, 1.23 g, 3.4 eq), DIPEA (6.49 mmol, 830 mg, 3.4 eq) and DMAP (20 mg, catalytic quantity) to obtain Lipid 32. Crude product was purified by column chromatography (4×) yielding 27 mg of pure fraction (>98% purity by LC-ELSD) of Lipid 32 and characterized by proton NMR and Mass Spectrometry (see FIG. 4S-1 for Lipid 32 NMR spectrum, FIG. 4S-2 for Lipid 32 LC-MS, and Table 5 for product mass).

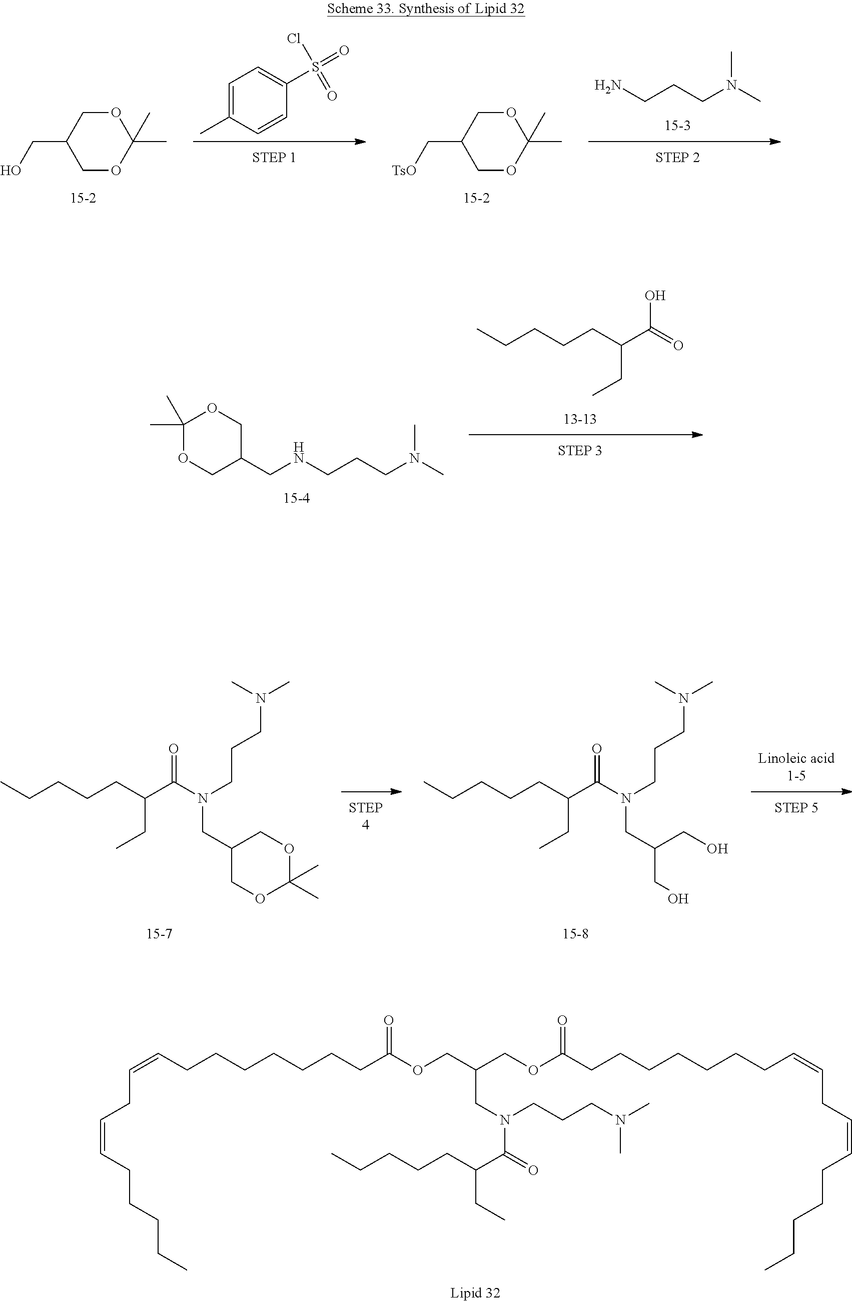

### Synthesis of Lipid 33

Lipid 33 was synthesized as provided in scheme 34 below and as follows. Starting material 15-1 (34.3 mmol, 5 g, 1 eq.) was tosylated using p-toluene sulfonyl chloride, TsCl (6.52 g, 1 eq, 34.3 mmol) using TEA (19.01 mL, 4 eq, 137 mmol) and DMAP (30 mg) in 200 mL DCM. Crude product was purified by column chromatography (1×) to obtain 10.2 g (98%) of reactive intermediate 15-2. Nucleophilic displacement of 15-2 (10.0 mmol, 2.3 g, 1 eq.) with diamine 15-9 (9.24 mmol, 1.0 mL, 1.2 eq.) in 10 mL dioxane (10 mL) yielded 1.6 g (97%) of compound 15-10. Nucleophilic displacement reaction was repeated using additional 15-2 (8.3 mmol, 2.5 g, 1 eq.) and diamine 15-9 (9.9 mmol, 1.1 mL, 1.2 eq.) in 50 mL dioxane for an additional quantity of compound 15-10. Crude products from the two reactions were purified by column chromatography to obtain a total of 1.7 g (˜50%) pure 15-10. N-acylation of 15-10 (4.05 mmol, 875 mg, 1 eq.) with nonanoic acid 13-12 (7.1 mmol, 1.24 mL, 1.8 eq.) using EDCI (1.4 g, 1.8 eq, 7.1 mmol), DIPEA (1.3 mL, 1.8 eq, 7.1 mmol) and DMAP (90 mg, 0.2 eq, 0.81 mmol) in 8 mL DCM yielded intermediate 15-11. Crude product was purified by column chromatography (2×) to obtain 230 mg (16%) of pure intermediate 15-11. Deprotection of 15-11 (0.64 mmol, 230 mg, 1 eq.) in 5 mL of 4M HCl in dioxane yielded intermediate 15-12. Crude product was purified by column chromatography (1×) to obtain 74 mg (37%) of pure intermediate 15-12. Intermediate 15-12 (0.24 mmol, 74 mg, 1 eq.) was acylated with linoleic acid 1-5 (169 mg, 2.5 eq, 0.58 mmol) using EDCI (120 mg, 2.5 eq, 0.58 mmol), DIPEA (102 μL, 2.5 eq, 0.58 mmol) and DMAP (6 mg, 0.2 eq, 0.048 mmol) in 5 mL DCM to obtain Lipid 33. Crude product was purified by column chromatography (2×) to obtain 64 mg (32%) of pure Lipid 33 (>99% purity by LC-ELSD) and characterized by proton NMR and Mass Spectrometry (see FIG. 4T-1 for Lipid 33 NMR spectrum, FIG. 4T-2 for Lipid 33 LC-MS, and Table 5 for product mass).

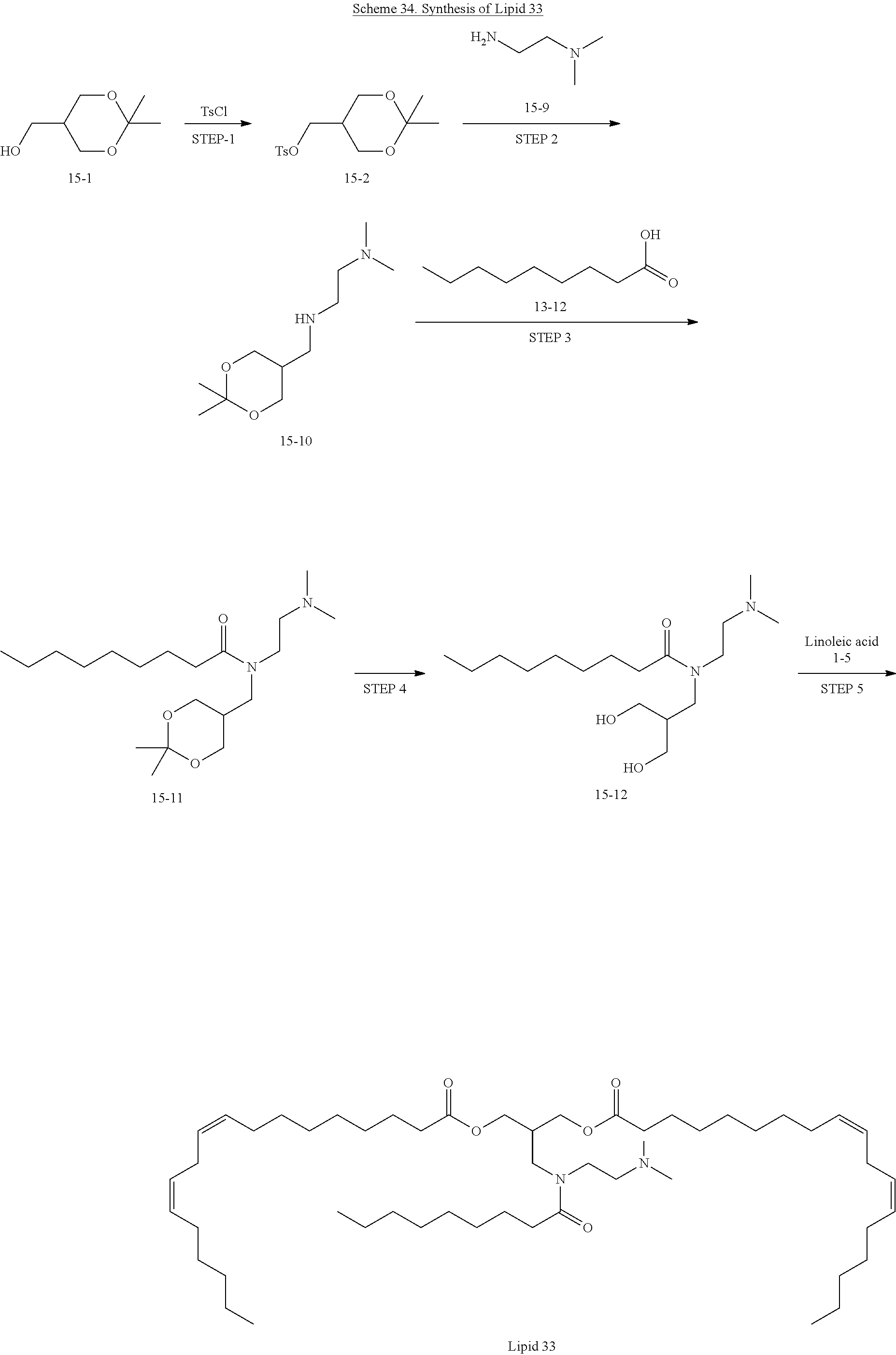

### Synthesis of Lipid 34

Lipid 34 was synthesized as provided in scheme 35 below and as follows. Intermediate 15-2 was accessed as described for Lipid 33. Nucleophilic displacement of 15-2 (3.3 mmol, 1 g, 1 eq.) with diamine 15-13 (3.9 mmol, 0.46 mL, 1.2 eq.) in 6 mL dioxane (10 mL) yielded 520 mg (64%) of compound 15-14. Reaction was repeated to access an additional 400 mg of pure compound 15-14. N-acylation of 15-14 (2.6 mmol, 620 mg, 1 eq.) with nonanoic acid 13-12 (5.3 mmol, 915 μL, 2.0 eq.) using EDCI (5.3 mmol, 1.06 g, 2.0 eq.), DIPEA (923 μL, 2.0 eq, 5.3 mmol) and DMAP (58 mg, 0.2 eq, 0.05 mmol) in 10 mL DCM yielded intermediate 15-15. Crude product was purified by column chromatography (2×) to obtain 355 mg (35%) of pure intermediate 15-15. Deprotection of 15-15 (1.03 mmol, 355 mg, 1 eq.) in 7 mL of 4M HCl in dioxane yielded intermediate 15-16. Crude product was purified by column chromatography (2×) to obtain 40 mg (13%) of pure intermediate 15-16. Intermediate 15-16 (0.116 mmol, 40 mg, 1 eq.) was acylated with linoleic acid 1-5 (81 mg, 2.5 eq, 0.29 mmol) using EDCI (55 mg, 2.5 eq, 0.29 mmol), DIPEA (3.2 μL, 2.5 eq, 0.29 mmol) and DMAP (2 mg, 0.2 eq, 0.05 mmol) in 10 mL DCM to obtain Lipid 34. Crude product was purified by column chromatography (2×) to obtain 73 mg (73%) of pure Lipid 34 (>99% purity by LC-ELSD) and characterized by proton NMR and Mass Spectrometry (see FIG. 4U-1 for Lipid 34 NMR spectrum, FIG. 4U-2 for Lipid 34 LC-MS, and Table 5 for product mass).

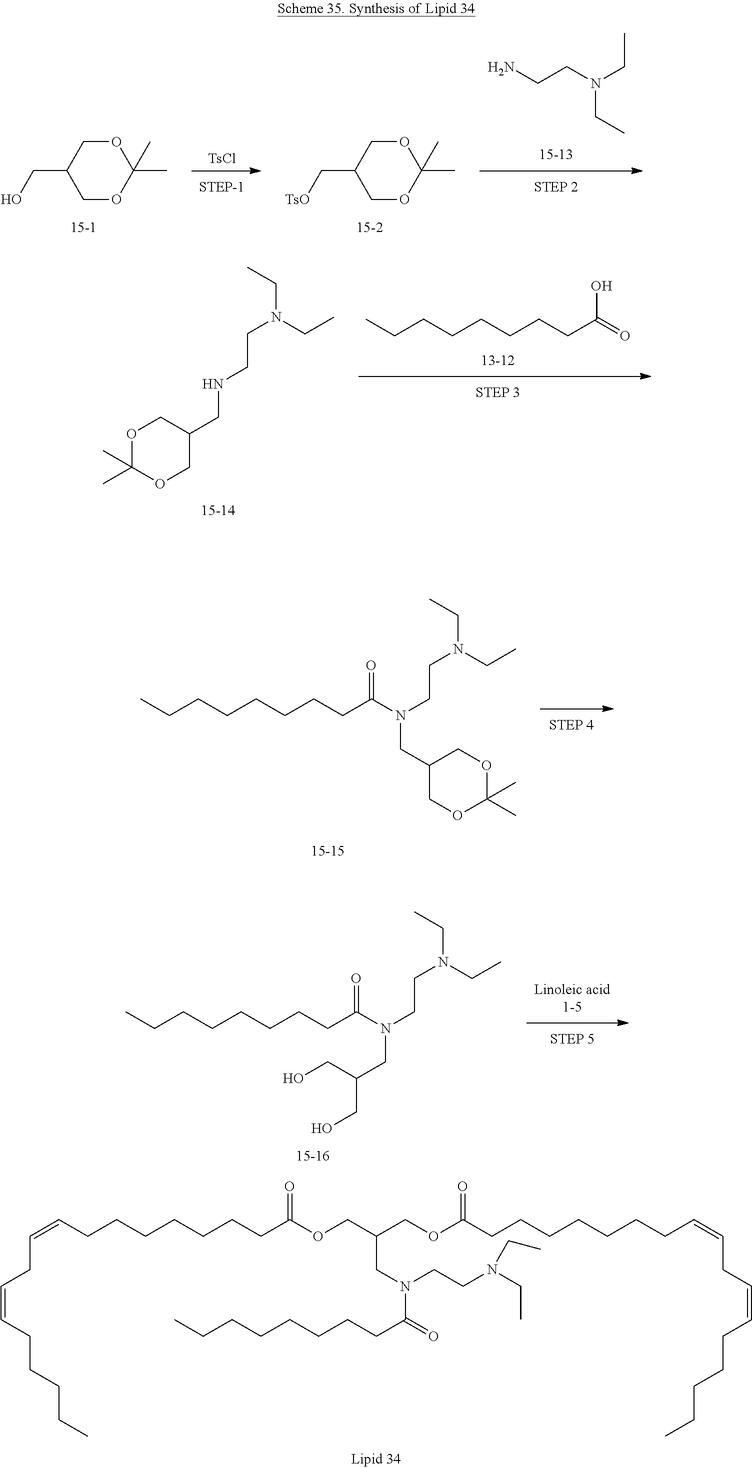

### Synthesis of Lipid 35

Lipid 35 was synthesized as provided in scheme 36 below.

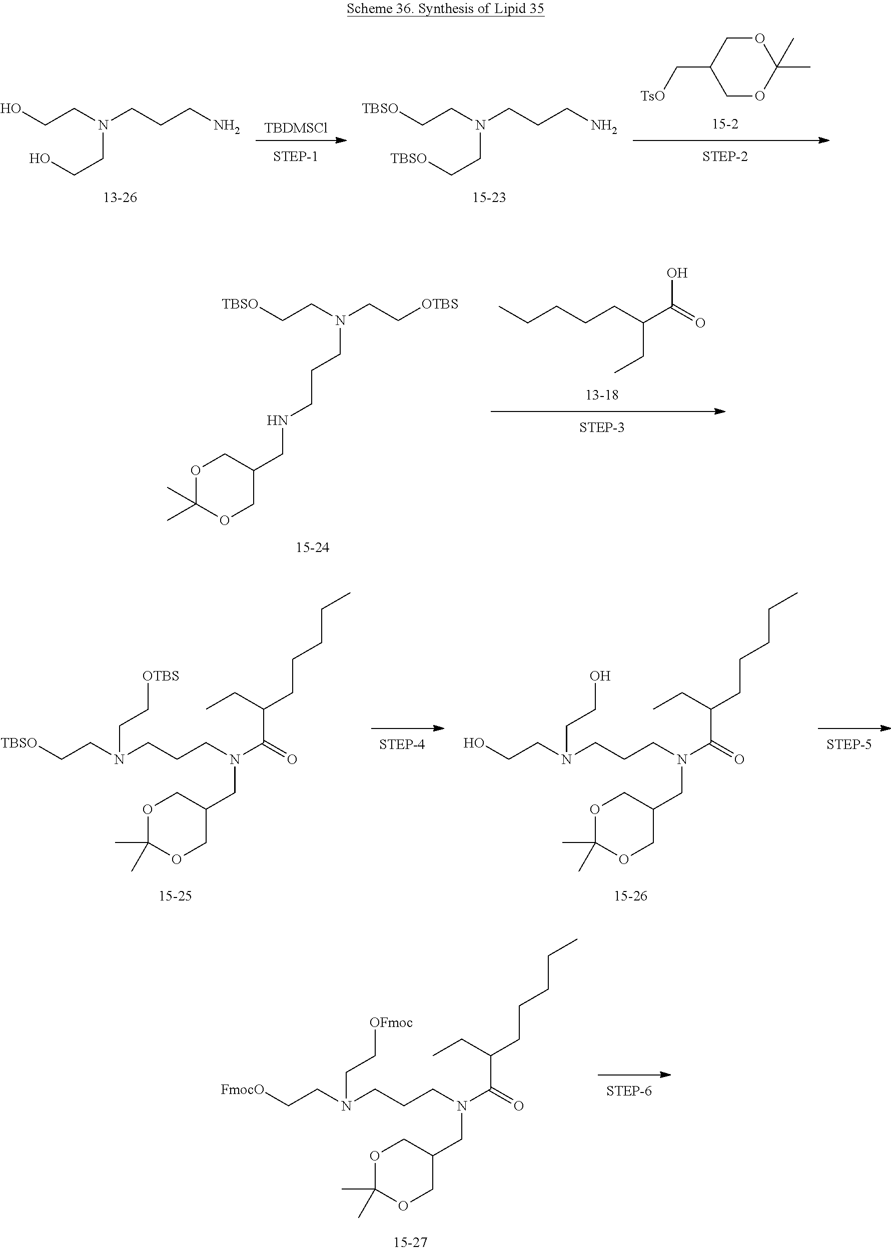

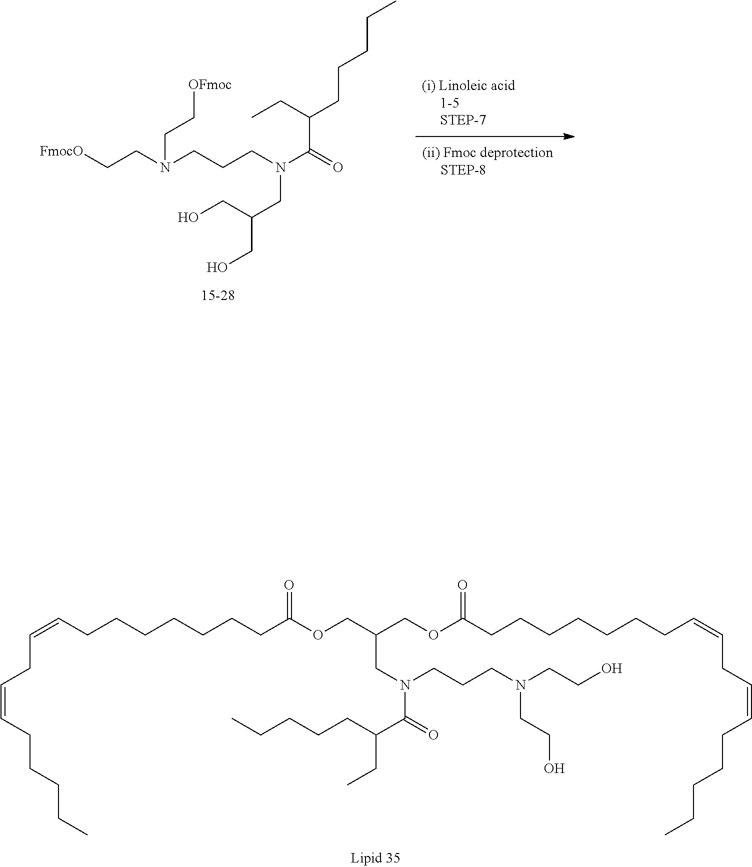

### Synthesis of Lipid 36

Lipid 36 was synthesized as provided in scheme 37 below.

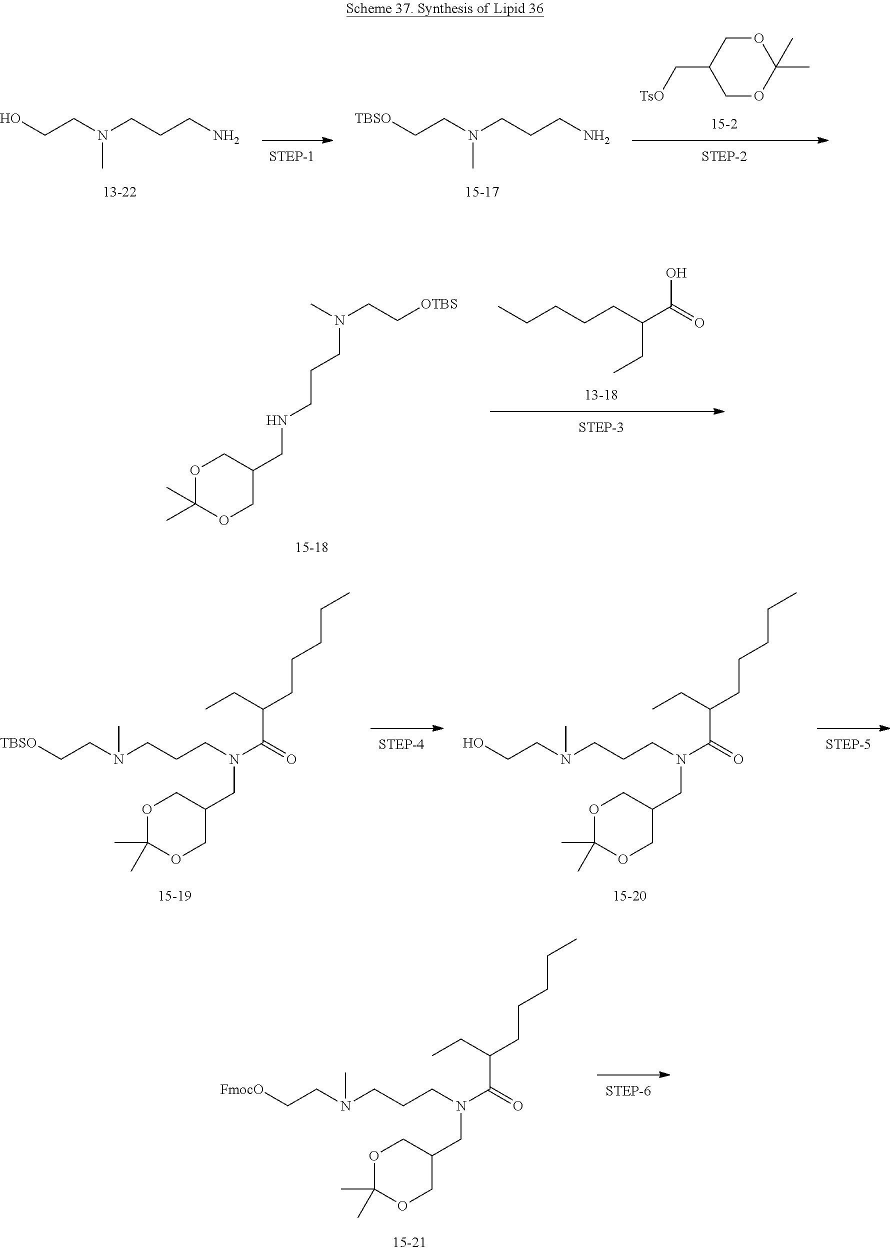

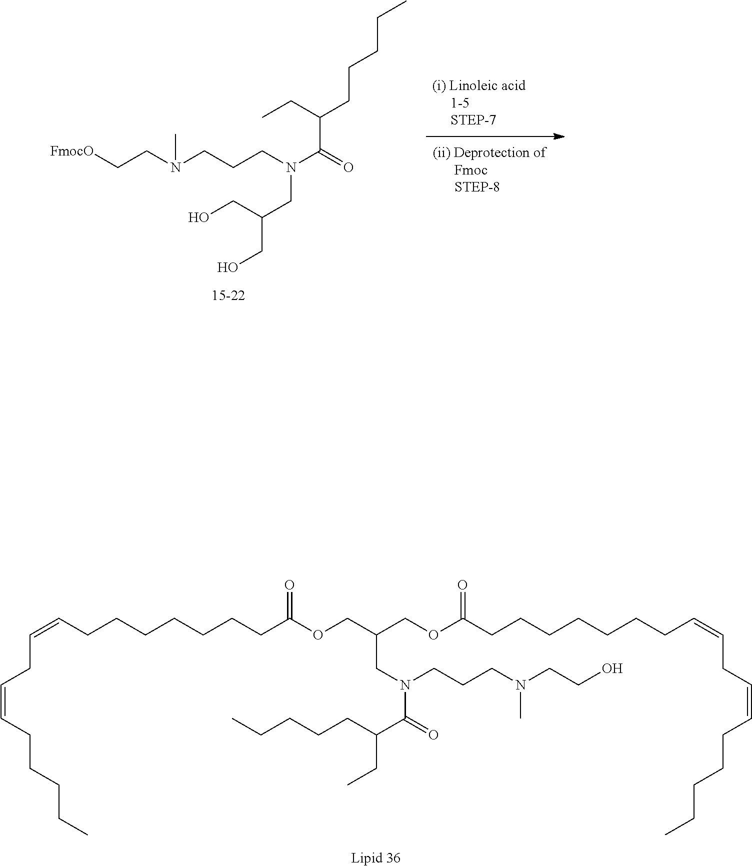

### Synthesis of Lipid 37A

Benzyl protected malonic acid 13-32 (5.1 mmol, 1.0 g, 1 eq.) was esterified with N-hexanol (0.78 g, 1.5 eq, 7.7 mmol) using HATU (2.93 g, 1.5 eq, 7.7 mmol) and DBU (1.56 g, 2.0 eq, 10.3 mmol) in 8 mL DMF for 16 hours at room temperature yielding 0.5 g (35%) of protected intermediate ester 14-9. A second 5 g scale batch using the same reaction conditions and reagent stoichiometry yielded an additional 6 g (84%) of intermediate 14-9. Intermediate 14-18 was produced via acylation of bromoacetyl bromide 13-35 with N-decanol.

Intermediate 14-9 (21.6 mmol, 6 g, 1 eq.) was alkylated with 14-18 (7.2 g, 1.2 eq, 26.0 mmol) using sodium hydride NaH (1.0 g, 1.2 eq, 26.0 mmol) in 40 mL DMF at room temperature overnight to obtain protected intermediate 14-19 (2.5 g, 25%). Intermediate 14-19 (2.5 g, 1.0 eq.) was deprotected using Pd/C (500 mg, 32% w/w) in 30 mL Ethyl Acetate at room temperature overnight yielding 2.02 g (99%) of acid intermediate 14-20.

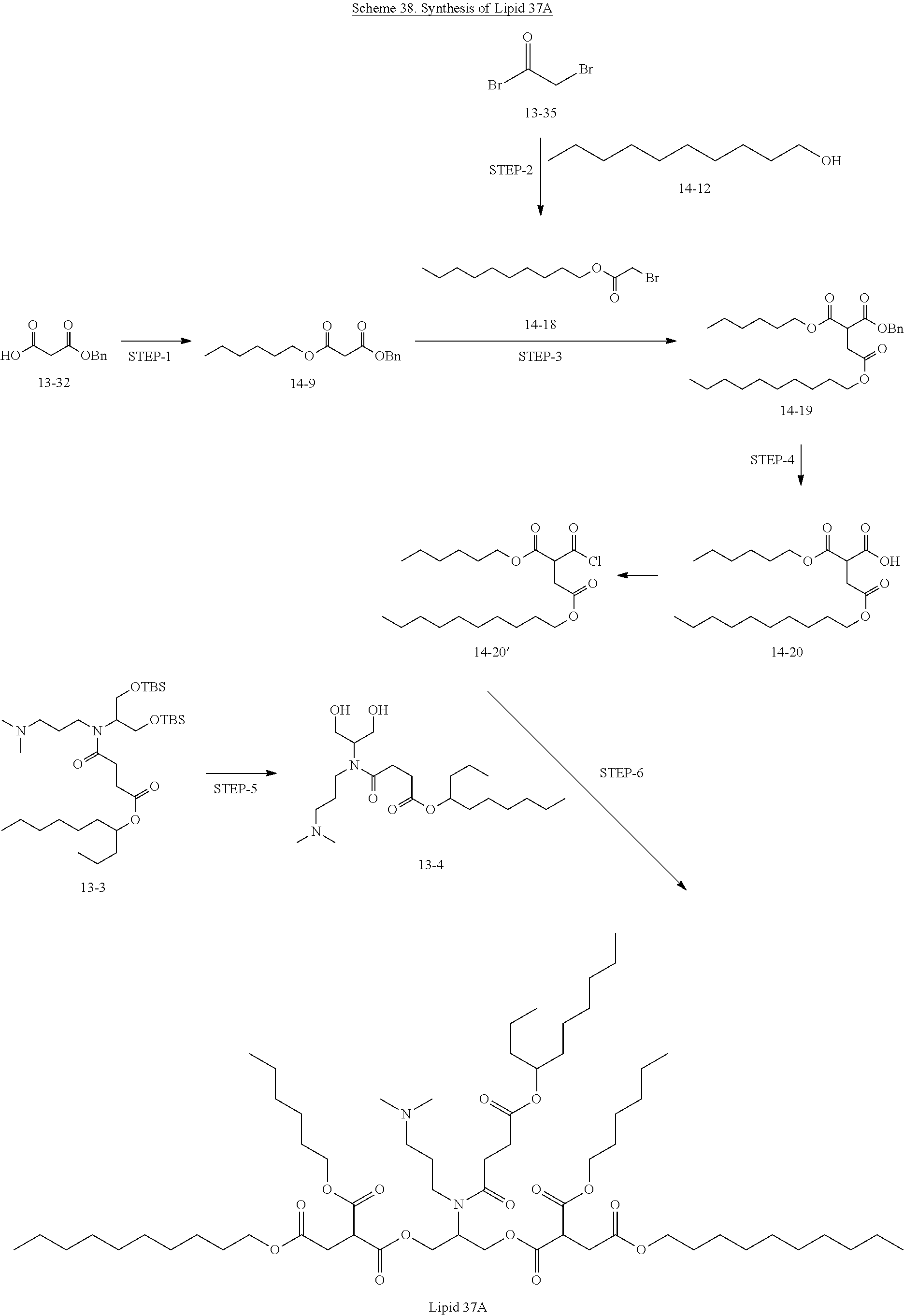

Acid intermediate 14-20 (2.7 mmol, 1.05 g, 1 eq.) was converted to the corresponding acid chloride 14-20′ using oxalyl chloride (0.79 mL, 3.4 eq, 9.2 mmol) and DMF (100 μL, catalytic quantity) in 6.0 mL of Toluene at room temperature for 2 hours and conversion to chloride intermediate was confirmed by TLC.

Intermediate 13-4 (1.57 mmol, 195 mg) was reacted with crude 14-20′ (2.83 g, 6.0 eq, 9.4 mmol) using TEA (2.18 mL, 10.0 eq, 15.7 mmol) in 16.0 mL of Toluene at room temperature overnight. Crude product was purified on ISCO column chromatography on silica column eluting with DCM and 10% MeOH in DCM twice to obtain 205 mg (38%) of >99% pure (HPLC-CAD) Lipid 37A. (see FIG. 4AH-1 and FIG. 4AH-2 for characterization by proton NMR and LC-CAD purity, and Table 5 for Mass Spectrometry data).

### Example 2. Preparation of LNPs by Microfluidic Mixing Using Exemplary Ionizable Lipids

Exemplary LNPs were produced using cationic Lipid 9 and cationic Lipid 15 as synthesized in Example 1.

LNPs encapsulating an mRNA payload were prepared by mixing an aqueous mRNA solution and an ethanolic lipid blend solution (containing ionizable lipid, DSPC, DPG-PEG and Cholesterol at lipid ratios shown in Table 6) using an in-line microfluidic mixing process. The mRNA (eGFP encoding mRNA, TriLink Biotechnologies, California, US) stock solution (1 mg/mL) was diluted in pH 4 acetate buffer (yielding a 133 μg/mL solution of mRNA) in 21.7 mM pH 4 acetate buffer. The lipid components were dissolved in anhydrous ethanol at the relative ratios set forth in TABLE 6 below.

TABLE 6

| | | Ratio of | | |
|---|---|---|---|---|
| | | Lipid to | Concen- | Theoretical |
| | | mRNA | tration | LNP Lipid |
| | | (nmol lipid/ | in Lipid | Compo- |
| | | 100 μg | Solution | sition |
| Lipid | Source | mRNA) | (mM) | (mol %) |
| Ionizable | — | 1,500 | 6 | 49.2 |
| Cationic Lipid | | | | |
| Cholesterol | Dishman | 1,200 | 4.8 | 39.4 |
| | Netherlands | | | |
| DSPC | Avanti Polar | 300 | 1.2 | 9.8 |
| | Lipids, Alabama, | | | |
| | U.S. | | | |
| DPG-PEG | NOF America, | 46 | 0.18 | 1.5 |
| (2000) | New York, U.S. | | | |
| DiIC18(5)-DS | Invitrogen, | 1.8 | 0.007 | 0.06 |
| | Massachusetts, US | | | |

The mRNA and lipid solutions were mixed using a NanoAssemblr Ignite microfluidic mixing device (part no. NIN0001) and NxGen mixing cartridge (part no. NIN0002) from Precision Nanosystems Inc. (British Columbia, CA). Briefly, the mRNA and lipid solutions were each loaded into separate polypropylene syringes. A mixing cartridge was inserted into the NanoAssemblr Ignite, and the syringes were directed mounted into the luer ports of the mixing cartridge. The two solutions were then mixed at a 3:1 v/v ratio of mRNA solution (1.5 mL) to lipid solution (0.5 mL) at a total flow rate of 9 mL/min using the NanoAssemblr Ignite. The resulting suspension was held at room temperature for a minimum of 5 minutes before proceeding to ethanol removal and buffer exchange.

Following mixing, ethanol removal and buffer exchange was performed on the resulting LNP suspension using a discontinuous diafiltration process. A centrifugal ultrafiltration device with 100,000 kDa MWCO regenerated cellulose membrane (Amicon Ultra-15, MilliporeSigma, Massachusetts, US) was sanitized with 70% ethanol solution and then washed twice with HBS exchange buffer (25 mM pH 7.4 HEPES buffer with 150 mM NaCl). The LNP suspension (2 mL) was then loaded into the device and centrifuged at 500 RCF until the volume was reduced by half volume (1 mL). The suspension was then diluted with exchange buffer (1 mL, 25 mM pH 7.4 HEPES buffer) to bring the suspension back to the original volume. This process of two-fold concentration and two-fold dilution was repeated five additional times for a total of six discontinuous diafiltration steps. The LNP suspension was then exchanged into MBS (25 mM pH 6.5 MES buffer with 150 mL NaCl) by diluting ten-fold with MBS and centrifuging at 500 RCF until the volume was reduced by one tenth. This ten-fold dilution with MBS and ten-fold concentration step was repeated one more time. The retentate containing the LNPs in MBS was recovered from the centrifugal ultrafiltration device and stored at 4° C. until further use.

### Example 3. Characterization of LNPs

This Example describes the characterization of LNPs produced in Example 2.

Samples of the LNPs produced in Example 2 were characterized to determine the average hydrodynamic diameter, zeta potential, and mRNA content (total and dye-accessible mRNA). The hydrodynamic diameter was determined by dynamic light scattering (DLS) using a Zetasizer model ZEN3600 (Malvern Pananalytical, UK). The zeta potential was measured in 5 mM pH 5.5 MES buffer and 5 mM pH 7.4 HEPES buffer by laser Doppler electrophoresis using the Zetasizer.

RNA content of the nanoparticles is measured using Thermo Fisher Quant-iT RiboGreen RNA Assay Kit. Dye accessible RNA, which includes both un-encapsulated RNA and accessible RNA at the LNP surface, is measured by diluting the nanoparticles to approximately 1 μg/mL mRNA using HEPES buffered saline, and then adding Quant-iT reagent to the mixture. Total RNA content is measured by disrupting a nanoparticle suspension by dilution of the stock LNP batch (typically at ≥40 ug/mL RNA) in 0.5% Triton solution in HEPES buffered saline to obtain a 1 ug/mL RNA solution (final nominal concentration based on formulation input values) and subsequent heating at 60° C. for 30 minutes followed by addition of Quant-It reagent. RNA is quantified by measuring fluorescence at 485/535 nm, and concentration is determined relative to a contemporaneously run RNA standard curve. The results are set forth in TABLE 7.

TABLE 7

| | | DLS Z- | | Zeta | Zeta | |
|---|---|---|---|---|---|---|
| | | Avg. | | Potential at | Potential at | Dye- |
| Formulation | | Diameter | | pH 5.5 | pH 7.4 | Accessible |
| No. | Ionizable Lipid | (nm) | DLS PDI | (mV) | (mV) | mRNA (%) |
| 1 | Cationic Lipid 9 | 95 | 0.07 | 17 | 0.0 | 5 |
| 2 | Cationic Lipid 15 | 98 | 0.06 | 20 | 0.3 | 5 |

### Example 4. Preparation of Fab Conjugates to Enable T-Cell Targeting

This Example describes the production of an exemplary lipid-immune cell targeting group conjugate.

An anti-CD3 Fab (hSP34 with mouse lambda and human lambda) (see amino acid sequences below) was conjugated to DSPE-PEG (2k)-maleimide via covalent coupling between the maleimide group and a C-terminal cysteine in the heavy chain (HC). Anti-CD3 Fab clone Hu291, anti-CD8 Fab clone TRX2, anti-CD8 Fab clone OKT8, a non-functional mutated OKT8 (mutOKT8), anti-CD4 Fab from Ibalizumab sequence, anti-CD5 Fab clone He3, anti-CD7 Fab clone TH-69, anti-CD2 Fab clone TS2/18.1, anti-CD2 Fab clone 9.6, anti-CD2 Fab clone 9-1 with human kappas were also conjugated using similar methods described herein. The protein (3-4 mg/mL), after buffer exchange into oxygen free, pH 7 phosphate buffer, was reduced in 2 mM TCEP in oxygen free pH 7 phosphate buffer for 1 hour at room temperature. The reduced protein was isolated using a 7 kDa SEC column to remove TCEP and buffer exchanged into fresh oxygen free pH 7 phosphate buffer.

The conjugation reaction was initiated by addition of a 10 mg/mL micellar suspension of DSPE-PEG-maleimide (Avanti Polar Lipids, Alabama, US) and 30 mg/mL DSPE-PEG-OCH₃ (Avanti Polar Lipids, Alabama, U.S.) (1:1 to 1:3 weight ratio is used depending on protein) in oxygen free pH 5.7 citrate buffer (1 mM Citrate). Protein solution is concentrated to 3-4 mg/mL using a 10 kDa Regenerated Cellulose Membrane and subsequently buffer exchanged in oxygen free pH 7 phosphate buffer using a 40 kDa Size Exclusion Column. The conjugation reaction is carried out using 2-4 mg/mL protein and a 3.5 molar excess of maleimide at 37° C. for 2 hours followed by incubation at room temperature for an additional 12-16 hours.

The production of the resulting conjugate was monitored by HPLC and the reaction quenched in 2 mM cysteine. The resulting conjugate (DSPE-PEG (2k)-anti-hSP34 Fab) is isolated using a 100 kDa Millipore Regenerated Cellulose membrane filtration using pH 7.4 HEPES buffer saline (25 mM HEPES, 150 mM NaCl) buffer and stored at 4° C. prior to use. After quenching, the final micelle composition consists of a mixture of DSPE-PEG-Fab, DSPE-PEG-maleimide (cysteine terminated), and DSPE-PEG-OCH₃. The ratio of the three components is DSPE-PEG-Fab:DSPE-PEG-maleimide (cysteine terminated):DSPE-PEG-OCH₃=1:2.45:3.45-10.35 (by mole)).

The resulting conjugate displayed comparable binding to recombinant Rhesus CD3 epsilon as the unconjugated anti-CD3 Fab by ELISA assay.

Anti-CD3 hSP34-Fab sequence:
hSP34 HC (SEQ ID NO: 1):
EVOLVESGGGLVQPGGSLKLSCAASGFTFNKYAMNWVRQAPGKGLEWVARIRSKY
NNYATYYADSVKDRFTISRDDSKNTAYLQMNNLKTEDTAVYYCVRHGNFGNSYISY
WAYWGQGTLVTVSSASTKGPSVFPLAPSSKSTSGGTAALGCLVKDYFPEPVTVSWN
SGALTSGVHTFPAVLQSSGLYSLSSVVTVPSSSLGTQTYICNVNHKPSNTKVDKKVEP
KSSDKTHTC
hSP34-mlam LC (mouse lambda) (SEQ ID NO: 2):
QTVVTQEPSLTVSPGGTVTLTCGSSTGAVTSGNYPNWVQQKPGQAPRGLIGGTKFLA
PGTPARFSGSLLGGKAALTLSGVQPEDEAEYYCVLWYSNRWVFGGGTKLTVLGQPK
SSPSVTLFPPSSEELETNKATLVCTITDFYPGVVTVDWKVDGTPVTQGMETTQPSKQS
NNKYMASSYLTLTARAWERHSSYSCQVTHEGHTVEKSLSRADSS
SP34-hlam LC (human lambda) (SEQ ID NO: 3):
QTVVTQEPSLTVSPGGTVTLTCGSSTGAVTSGNYPNWVQQKPGQAPRGLIGGTKFLA
PGTPARFSGSLLGGKAALTLSGVQPEDEAEYYCVLWYSNRWVFGGGTKLTVLSQPK
AAPSVTLFPPSSEELQANKATLVCLVSDFYPGAVTVAWKADGSPVKVGVETTKPSK
QSNNKYAASSYLSLTPEQWKSHRSYSCRVTHEGSTVEKTVAPAESS
Anti-CD8 TRX2-Fab sequence:
TRX2 HC (SEQ ID NO: 6):
QVQLVESGGGVVQPGRSLRLSCAASGFTFSDFGMNWVRQAPGKGLEWVALIYYDG
SNKFYADSVKGRFTISRDNSKNTLYLQMNSLRAEDTAVYYCAKPHYDGYYHFFDS
WGQGTLVTVSSASTKGPSVFPLAPSSKSTSGGTAALGCLVKDYFPEPVTVSWNSGAL
TSGVHTFPAVLQSSGLYSLSSVVTVPSSSLGTQTYICNVNHKPSNTKVDKKVEPKSSD
KTHTC
TRX2 LC (SEQ ID NO: 7):
DIQMTQSPSSLSASVGDRVTITCKGSQDINNYLAWYQQKPGKAPKLLIYNTDILHTG
VPSRFSGSGSGTDFTFTISSLQPEDIATYYCYQYNNGYTFGQGTKVEIKRTVAAPSVFI
FPPSDEQLKSGTASVVCLLNNFYPREAKVQWKVDNALQSGNSQESVTEQDSKDSTY
SLSSTLTLSKADYEKHKVYACEVTHQGLSSPVTKSFNRGES
Anti-CD8 OKT8-Fab sequence:
OKT8 HC (SEQ ID NO: 8):
QVQLVQSGAEDKKPGASVKVSCKASGFNIKDTYIHWVRQAPGQGLEWMGRIDPAN
DNTLYASKFQGRVTITADTSSNTAYMELSSLRSEDTAVYYCGRGYGYYVFDHWGQ
GTTVTVSSASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKKVEPKSSDKTH
TC
OKT8 LC (SEQ ID NO: 9):
DIVMTQSPSSLSASVGDRVTITCRTSRSISQYLAWYQEKPGKAPKLLIYSGSTLQSGVP
SRFSGSGSGTDFTLTISSLQPEDFATYYCQQHNENPLTFGQGTKVEIKRTVAAPSVFIF
PPSDEQLKSGTASVVCLLNNFYPREAKVQWKVDNALQSGNSQESVTEQDSKDSTYS
LSSTLTLSKADYEKHKVYACEVTHQGLSSPVTKSFNRGES
mutOKT8-Fab sequence:
mutOKT8 HC (SEQ ID NO: 22):
QVQLVQSGAEDKKPGASVKVSCKASGFNIKDTYIHWVRQAPGQGLEWMGRIDPAN
DNTLYASKFQGRVTITADTSSNTAYMELSSLRSEDTAVYYCGRGAGAYVFDHWGQ
GTTVTVSSASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKKVEPKSSDKTH
TC
mutOKT8 LC (SEQ ID NO: 23):
DIVMTQSPSSLSASVGDRVTITCRTSRSISAALAWYQEKPGKAPKLLIYSGSTLQSGVP
SRFSGSGSGTDFTLTISSLQPEDFATYYCQQHNENPLTFGQGTKVEIKRTVAAPSVFIF
PPSDEQLKSGTASVVCLLNNFYPREAKVQWKVDNALQSGNSQESVTEQDSKDSTYS
LSSTLTLSKADYEKHKVYACEVTHQGLSSPVTKSFNRGES

### Example 5. Preparation of LNPs Containing T Cell Targeting Group

This Example describes the incorporation of an immune cell targeting conjugate into a preformed LNP.

LNPs from Example 2 and conjugates (anti-CD3 (hSP34) and anti-CD8 (TRX-2) conjugates) were prepared using methods described in Example 4 were combined as shown in Table 8 in an Eppendorf tube and vortexed for 10 seconds at 2,500 rpm. The Eppendorf tubes were placed in the ThermoMixer at 37° C. at 300 rpm for 4 hours. Resulting targeted LNP suspension was subsequently stored at 4° C. until use or alternatively stored frozen after reconstitution into sucrose medium at final sucrose concentration of 9.6 wt. % by dilution using the appropriate volume of a 50 wt. % sucrose stock solution (in HEPES buffer saline; 25 mM HEPES, 150 mM NaCl)) and stored frozen at −80° C.

TABLE 8

| nmol | Target FAb | | RNA | FAb | | |
|---|---|---|---|---|---|---|
| total | density in | FAb | Conc. In | Conjugate | Fab | LNP |
| lipid/mg | targeted LNP g | mg/mg | LNP | Conc. | mg/mL | mL/mL |
| RNA | (Fab/mol lipid) | RNA | (mg/mL) | (mg/mL) | LNP | Fab |
| 30,460 | 9 | 0.274 | 0.45 | 1.46 | 0.123 | 11.8 |
| 30,460 | 3 | 0.091 | 0.45 | 1.46 | 0.041 | 35.5 |

### Example 6. Preparation of LNPs by Microfluidic In-Line Mixing and Tangential Flow Filtration Using an Exemplary Ionizable Lipid

This example describes preparation of LNPs using scalable unit operations, namely in-line microfluidic mixing followed by tangential flow filtration (TFF) for ethanol removal and buffer exchange. Separate LNP batches were prepared using either DLin-KC3-DMA or Lipid 15 as the ionizable lipid.

LNPs encapsulating an mRNA payload were prepared by mixing an aqueous mRNA solution and an ethanolic lipid blend solution using an in-line microfluidic mixing process. The mRNA (eGFP or mCherry encoding mRNA, TriLink Biotechnologies, California, US) stock solution was diluted in pH 4 acetate buffer (yielding a 400 μg/mL solution of mRNA) in 65 mM pH 4 acetate buffer. The lipid components were dissolved in anhydrous ethanol at the relative ratios set forth in TABLE 9.1 below.

TABLE 9.1

| | | Ratio of | | |
|---|---|---|---|---|
| | | Lipid to | Concen- | |
| | | mRNA | tration | Theoretical |
| | | (nmol lipid/ | in Lipid | LNP Lipid |
| | | 100 μg | Solution | Composition |
| Lipid | Source | mRNA) | (mM) | (mol %) |
| Ionizable | — | 1,500 | 18.0 | 49.2 |
| Cationic | | | | |
| Lipid | | | | |
| Cholesterol | Dishman | 1,200 | 14.4 | 39.4 |
| | Netherlands | | | |
| DSPC | Avanti Polar | 300 | 3.6 | 9.8 |
| | Lipids, Alabama, | | | |
| | U.S. | | | |
| DPG-PEG | NOF America, | 46 | 0.55 | 1.5 |
| (2000) | New York, U.S. | | | |

The mRNA and lipid solutions were mixed using a NanoAssemblr Ignite microfluidic mixing device (part no. NIN0001) and NxGen mixing cartridge (part no. NIN0002) from Precision Nanosystems Inc. (British Columbia, CA). Briefly, the mRNA and lipid solutions were each loaded into separate polypropylene syringes. A mixing cartridge was inserted into the NanoAssemblr Ignite, and the syringes were directed mounted into the luer ports of the mixing cartridge. The two solutions were then mixed at a 3:1 v/v ratio of mRNA solution to lipid solution at a total flow rate of 9 mL/min using the NanoAssemblr Ignite. The resulting suspension was held at room temperature for a minimum of 5 minutes before proceeding to ethanol removal and buffer exchange. Ethanol removal and buffer exchange were subsequently performed using constant volume tangential flow filtration (TFF).

Following mixing, ethanol removal and buffer exchange were performed on the resulting LNP suspension using a hollow fiber TFF module (mPES membrane with 300 kDa MWCO, Repligen, US). Briefly, the TFF module was rinsed with DI water and pumped dry before use. The buffer selected for use as the diafiltration buffer depended on the ionizable lipid in the LNP formulation. For DLin-KC3-DMA LNPs, 25 mM pH 7.4 HEPES buffer with 150 mM NaCl (HBS) was used as the diafiltration buffer. For Lipid 15 LNPs, 25 mM pH 6.5 MES buffer with 150 mM NaCl (MBS) was used as the diafiltration buffer. The LNP mixture in ethanol/water solution was added to the reservoir, the TFF module was primed, and diafiltration was initiated by ramping up the peristaltic pump to the target flow rate and adjusting the retentate valve until the target transmembrane pressure (TMP) was reached. A shear rate of 8000 s⁻¹ and a TMP of 3.5 psi were the target operating parameters for the system once diafiltration was initiated. Throughout the diafiltration process, the TMP was kept constant by adjusting the retentate valve. Permeate flux was monitored and was >20 LMH throughout diafiltration. Six diafiltration exchange volumes were performed, with samples set aside at the end of each diafiltration volume to later track the buffer exchange process. Final ethanol content was ≤0.1%, as measured by refractive index measurements on permeate samples, and pH measurements confirmed the buffer exchange into the desired diafiltration buffer. Upon the completion of six diafiltration volumes, a concentration of the resulting LNP suspension was subsequently performed.

The concentration of the LNP suspension to a target total mRNA concentration of ˜0.8 mg/mL was performed using the same TFF module that was used during the buffer exchange process. TMP and flow rate during the buffer exchange process were maintained, and the suspension was allowed to concentrate by stopping the addition of diafiltration buffer to the retentate reservoir. The resulting LNP suspension was collected and filtered with a 0.2 μm syringe filter. The suspension was sampled for analytical purposes and then stored at 4° C. until further use.

Using the LNP characterization process in Example 3, LNP batch was characterized to determine the average hydrodynamic diameter and mRNA content (total and dye-accessible); set forth in Table 9 below. As seen in Table 9.2, the microfluidic mixing process with ethanol removal and buffer exchange by TFF results in sub-100 nm particles exhibiting narrow polydispersity and good mRNA encapsulation (<20% dye accessible RNA).

TABLE 9.2

| | | | | | Dye- | |
|---|---|---|---|---|---|---|
| | | DLS Z- | | Total | Accessible | Dye- |
| | | Avg. | | mRNA | mRNA | Accessible |
| Sample ID/Lot | | Diameter | DLS | Content | Content | mRNA |
| Number | Description | (nm) | PDI | (μg/mL) | (μg/mL) | (%) |
| EXP22001562- | DLin-KC3-DMA | 85 | 0.07 | 846 | 115 | 14 |
| NF40 | LNPs after 6 DVs | | | | | |
| | in HBS | | | | | |
| EXP22007940- | Lipid 15 LNPs | 69 | 0.14 | 990 | 161 | 16 |
| NU400 | after 6 DVs in | | | | | |
| | MBS | | | | | |

### Example 7. Method for Determination of the LNP Apparent pKa Using the Toluidinyl-Naphthalene Sulfonate (TNS) Fluorescent Probe

This example describes the fluorescent dye based method used for measurement of the apparent pKₐ of the lipid nanoparticles. Apparent pKₐ determines the nanoparticle surface charge under physiological pH conditions, typically a pKa value in the endosomal pH range (6-7.4) results in LNPs that are neutral or slightly charged at plasma or the extracellular space (pH 7.4) and become strongly positive under acidic endosomal environments. This positive surface charge drives fusion of the LNP surface with negatively charged endosomal membranes resulting in destabilization and rupture of the endosomal compartment and LNP escape into the cytosolic compartment, a critical step in cytosolic delivery of mRNA and protein expression via engagement of the cells ribosomal machinery.

The apparent pKa of LNPs is determined by 6-(p-Toluidino)-2-naphthalenesulfonic acid (TNS) fluorescence measurement in aqueous buffers covering a range of pH values (pH 4-pH 10). TNS dye is non-fluorescent when free in solution, but fluoresces strongly when associated with a positively charged lipid nanoparticle. At pH values below the pKa of the nanoparticle, positive LNP surface charge results in dye recruitment at the particle interface resulting in TNS fluorescence. At pH values above the LNP pKa, the LNP surface charge is neutralized and TNS dye dissociates away from the particle interface resulting in loss of fluorescence signal. The apparent pKa of the LNP is reported as the pH at which the fluorescence is at 50% of its maximum, as determined using a four-point logistic curve fit.

### Example 8. General Formulation and Physiochemical Characterization Methodology for mRNA Encapsulating LNPs Based on Lipids 1-8, 9-15 and 31-34

Lipid nanoparticles (LNPs) bearing a nucleic acid (reporter RNA or CAR RNA) were formulated by a microfluidic mixing process using lipid and solvent compositions described in Example 2 and 6 above and buffer exchanged into pH 7.4 HEPES buffer saline (resulting in ethanol removal and pH adjustment) using either centrifugal ultrafiltration membrane filter devices or a tangential flow filtration (TFF) process; and characterized by Dynamic Light Scattering (DLS) for hydrodynamic size (diameter, nm), polydispersity (PDI) and charge at pH 5.5 and pH 7.4 (Zeta Potential, mV). The mRNA encapsulation efficiency (percent dye accessible RNA) and total mRNA content (μg/mL RNA in LNP suspension) were determined using methods described in Example 3. The formulated LNPs were subsequently buffer exchanged into pH 6.5 MES buffer saline and the size distribution was re-characterized by DLS prior to mixing with the desired quantity of targeting antibody conjugate (see Table 8, Example 5) and incubated at 37° C. for 4 hours to facilitate antibody insertion (using process described in Example 5) resulting in final antibody targeted LNPs. The obtained targeted LNPs were sterile filtered and characterized by DLS (size (nm) and PDI) using methods described in Example 3.

### Example 9. Physiochemical Properties (Pre- and Post-Insertion with αCD3 Fab Conjugate hSP34) of Lipids 1-8 GFP RNA Lipid Nanoparticles (LNPs)

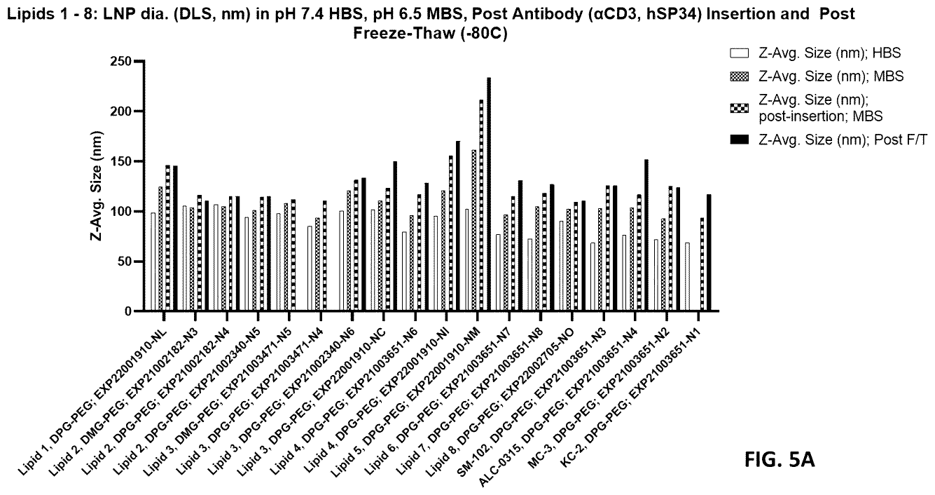

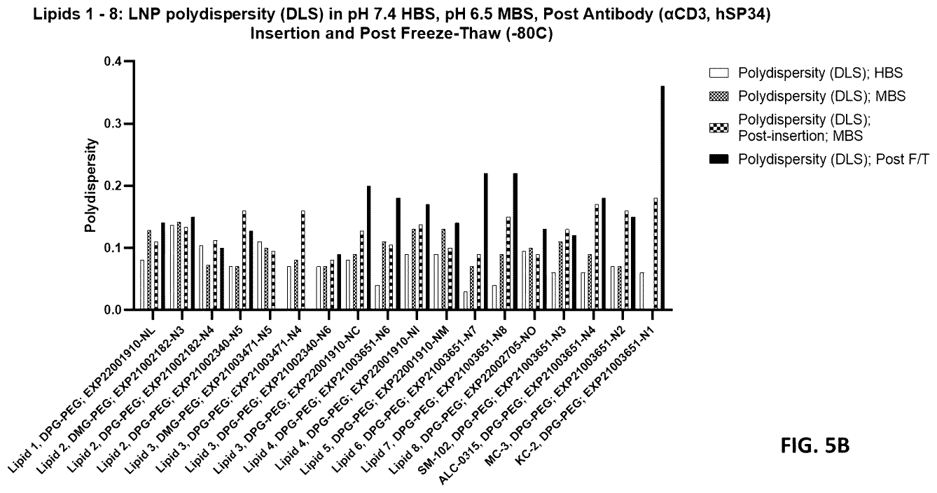

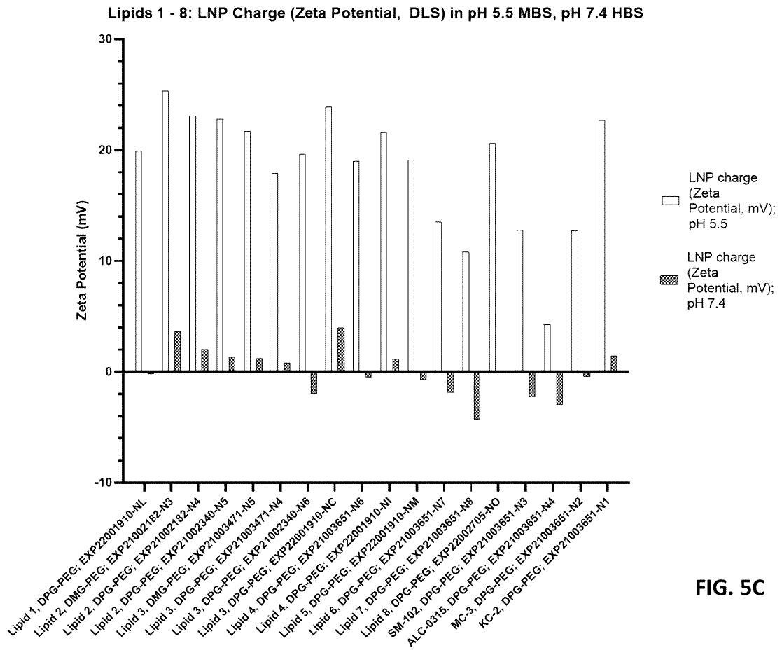

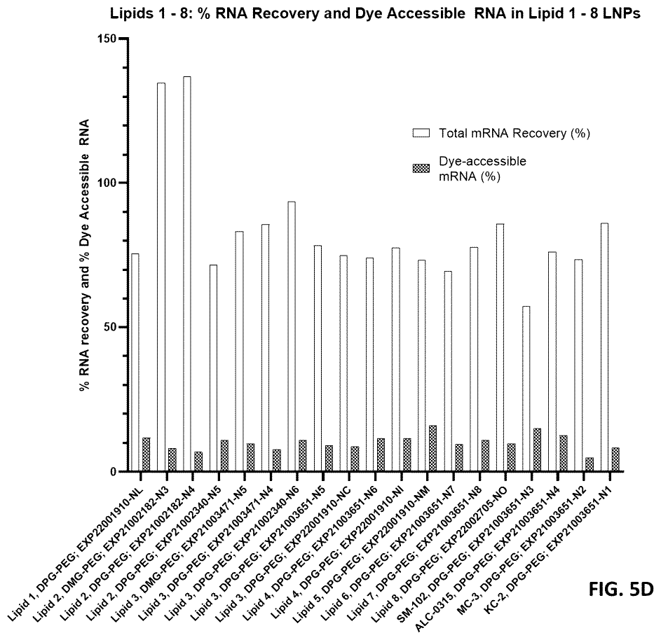

Lipids 1, 2, 3, 4, 5, 6, 7 and 8 LNPs encapsulating either GFP-mRNA (TriLink Biotechnologies Inc.) or CAR-RNA (custom made by TriLink Biotechnologies Inc.) were prepared and characterized using methods described in Examples 5 to 8. Measured LNP size, PDI, charge and RNA content values for Lipid 1-8 LNP and LNPs made using comparator ionizable lipids (including MC3, KC2, SM-102, and ALC-0315) are summarized in Table 10 to Table 12. As seen in FIG. 5A, the initial mixing step and subsequent buffer exchange into HBS resulted in Lipid 1-8 LNP sizes in the 80-120 nm range. All lipids resulted in high encapsulation efficiency (<15% Dye accessible RNA) and >70% RNA recovery. The subsequent buffer exchange into pH 6.5 MES buffer and antibody insertion process (37° C., 4 hours) was well tolerated by lipids 2, 3, 6, 7 and 8 LNPs resulting in targeted LNP diameters below 140 nm and PDI <0.2. Lipids 1 and 4 LNPs exhibited a relatively larger size shifts resulting in final targeted LNPs in the 140-160 nm diameter range, however particles size distributions remained narrow and monomodal for all lipids tested. Lipid 5 LNPs exhibited the greatest size shift with in final targeted LNPs exhibiting >160 nm diameter. Lipids 1, 2, 6, 7 and 8 exhibited no significant size and polydispersity changes after one Freeze-Thaw cycle, however, moderate changes were observed with LNPs based on lipids 3, 4, and 5. In summary, all lipids tested resulted in viable mRNA encapsulation and freeze-thaw stability as well as <200 nm final targeted LNP diameters. The ability of lipids 1-8 LNPs for inducing in vitro protein transfection in primary human T-cells mediated by αCD3 or αCD8 T-cell receptor targeting was evaluated as described in Examples 16 and 17.

TABLE 10
Lipids 1-8 and comparator Lipids LNP Size, Polydispersity (DLS) data in pH
7.4 HBS, pH 6.5 MBS and post αCD3 antibody Fab (hSP34) conjugate insertion

| | | | Z-Avg. | | | | | |
|---|---|---|---|---|---|---|---|---|
| | Z-Avg. | Z-Avg. | Size | Z-Avg. | | | Polydispersity | |
| Ionizable | Size | Size | (nm); | Size | Polydispersity | Polydispersity | (DLS); | Polydispersity |
| Lipid, LNP | (nm); | (nm); | post-insertion; | (nm); | (DLS); | (DLS); | Post-insertion; | (DLS); |
| no. | HBS | MBS | MBS | Post F/T | HBS | MBS | MBS | Post F/T |
| Lipid 1, | 98.78 | 124.3 | 146.2 | 145.5 | 0.08 | 0.128 | 0.11 | 0.14 |
| DPG-PEG; | | | | | | | | |
| EXP22001910-NL | | | | | | | | |
| Lipid 2, | 105.4 | 103.95 | 116.5 | 110.5 | 0.136 | 0.142 | 0.133 | 0.15 |
| DMG-PEG; | | | | | | | | |
| EXP21002182-N3 | | | | | | | | |
| Lipid 2, | 107 | 105 | 115.2 | 115.1 | 0.104 | 0.072 | 0.112 | 0.1 |
| DPG-PEG; | | | | | | | | |
| EXP21002182-N4 | | | | | | | | |
| Lipid 2, | 94 | 101.3 | 114.7 | 114.8 | 0.07 | 0.07 | 0.16 | 0.127 |
| DPG-PEG; | | | | | | | | |
| EXP21002340-N5 | | | | | | | | |
| Lipid 3, | 98.1 | 107.8 | 111.8 | | 0.11 | 0.1 | 0.095 | |
| DMG-PEG; | | | | | | | | |
| EXP21003471-N5 | | | | | | | | |
| Lipid 3, | 85.3 | 93.7 | 110.4 | | 0.07 | 0.08 | 0.16 | |
| DPG-PEG; | | | | | | | | |
| EXP21003471-N4 | | | | | | | | |
| Lipid 3, | 100.7 | 120.5 | 131.3 | 133.3 | 0.07 | 0.07 | 0.08 | 0.09 |
| DPG-PEG; | | | | | | | | |
| EXP21002340-N6 | | | | | | | | |
| Lipid 3, | 101.5 | 110.8 | 123.6 | 150.2 | 0.08 | 0.09 | 0.127 | 0.2 |
| DPG-PEG; | | | | | | | | |
| EXP22001910-NC | | | | | | | | |
| Lipid 4, | 79.5 | 96.3 | 116.8 | 128.5 | 0.04 | 0.11 | 0.105 | 0.18 |
| DPG-PEG; | | | | | | | | |
| EXP21003651-N6 | | | | | | | | |
| Lipid 4, | 95.5 | 120.6 | 155.4 | 170.3 | 0.09 | 0.13 | 0.137 | 0.17 |
| DPG-PEG; | | | | | | | | |
| EXP22001910-NI | | | | | | | | |

TABLE 11
Lipids 1-8 LNP charge, Zeta Potential, ZP (DLS, mV)
in pH 5.5 MES and pH 7.4 HBS

| | Charge | Charge |
|---|---|---|
| | (ZP, mV); | (ZP, mV); |
| Ionizable Lipid, LNP no. | pH 5.5 | pH 7.4 |
| Lipid 1, DPG-PEG; EXP22001910-NL | 19.9 | −0.212 |
| Lipid 2, DMG-PEG; EXP21002182-N3 | 25.3 | 3.64 |
| Lipid 2, DPG-PEG; EXP21002182-N4 | 23.1 | 2.03 |
| Lipid 2, DPG-PEG; EXP21002340-N5 | 22.8 | 1.29 |
| Lipid 3, DMG-PEG; EXP21003471-N5 | 21.7 | 1.18 |
| Lipid 3, DPG-PEG; EXP21003471-N4 | 17.9 | 0.776 |
| Lipid 3, DPG-PEG; EXP21002340-N6 | 19.6 | −2.01 |
| Lipid 3, DPG-PEG; EXP22001910-NC | 23.9 | 3.99 |
| Lipid 4, DPG-PEG; EXP21003651-N6 | 19 | −0.508 |
| Lipid 4, DPG-PEG; EXP22001910-NI | 21.6 | 1.16 |
| Lipid 5, DPG-PEG; EXP22001910-NM | 19.1 | −0.742 |
| Lipid 6, DPG-PEG; EXP21003651-N7 | 13.5 | −1.9 |
| Lipid 7, DPG-PEG; EXP21003651-N8 | 10.8 | −4.31 |
| Lipid 8, DPG-PEG; EXP22002705-NO | 20.6 | 0.051 |
| SM-102, DPG-PEG; EXP21003651-N3 | 12.8 | −2.25 |
| ALC-0315, DPG-PEG; EXP21003651-N4 | 4.25 | −2.95 |
| MC-3, DPG-PEG; EXP21003651-N2 | 12.7 | −0.43 |
| KC-2, DPG-PEG; EXP21003651-N1 | 22.7 | 1.41 |

TABLE 12
Lipids 1-8 and comparator Lipids LNP Dye Accessible RNA and total
RNA content

| | Nominal | | Dye- | | |
|---|---|---|---|---|---|
| | mRNA | Measured | accessible | Total | Dye- |
| Ionizable Lipid, | Conc. | Total mRNA | mRNA | mRNA | accessible |
| LNP no. | (μg/mL) | (μg/mL) | (μg/mL) | Recovery (%) | mRNA (%) |
| Lipid 1, DPG-PEG; | 100 | 75.5 | 8.9 | 75.5 | 11.8 |
| EXP22001910-NL | | | | | |
| Lipid 2, DMG- | 100 | 134.7 | 11.1 | 134.7 | 8.2 |
| PEG; | | | | | |
| EXP21002182-N3 | | | | | |
| Lipid 2, DPG-PEG; | 100 | 136.9 | 9.4 | 136.9 | 6.9 |
| EXP21002182-N4 | | | | | |
| Lipid 2, DPG-PEG; | 100 | 71.8 | 7.8 | 71.8 | 10.9 |
| EXP21002340-N5 | | | | | |
| Lipid 3, DMG- | 100 | 83.2 | 8.1 | 83.2 | 9.8 |
| PEG; | | | | | |
| EXP21003471-N5 | | | | | |
| Lipid 3, DPG-PEG; | 100 | 85.7 | 6.6 | 85.7 | 7.7 |
| EXP21003471-N4 | | | | | |
| Lipid 3, DPG-PEG; | 100 | 93.5 | 10.3 | 93.5 | 11.0 |
| EXP21002340-N6 | | | | | |
| Lipid 3, DPG-PEG; | 100 | 74.9 | 6.5 | 74.9 | 8.7 |
| EXP22001910-NC | | | | | |
| Lipid 4, DPG-PEG; | 50 | 37.1 | 4.3 | 74.2 | 11.5 |
| EXP21003651-N6 | | | | | |
| Lipid 4, DPG-PEG; | 100 | 77.6 | 9 | 77.6 | 11.6 |
| EXP22001910-NI | | | | | |
| Lipid 5, DPG-PEG; | 100 | 73.4 | 11.8 | 73.4 | 16.1 |
| EXP22001910-NM | | | | | |
| Lipid 6, DPG-PEG; | 50 | 34.7 | 3.3 | 69.4 | 9.5 |
| EXP21003651-N7 | | | | | |
| Lipid 7, DPG-PEG; | 50 | 38.9 | 4.3 | 77.8 | 11.1 |
| EXP21003651-N8 | | | | | |
| Lipid 8, DPG-PEG; | 100 | 85.9 | 8.4 | 85.9 | 9.8 |
| EXP22002705-NO | | | | | |
| SM-102, DPG- | 50 | 28.7 | 4.3 | 57.4 | 15.0 |
| PEG; | | | | | |
| EXP21003651-N3 | | | | | |
| ALC-0315, DPG- | 50 | 38.1 | 4.8 | 76.2 | 12.6 |
| PEG; | | | | | |
| EXP21003651-N4 | | | | | |
| MC-3, DPG-PEG; | 50 | 36.8 | 1.8 | 73.6 | 4.9 |
| EXP21003651-N2 | | | | | |
| KC-2, DPG-PEG; | 50 | 43 | 3.6 | 86 | 8.4 |
| EXP21003651-N1 | | | | | |

### Example 10. Physiochemical Properties (Pre- and Post-Insertion with αCD3 Fab Conjugate Hsp34) of Lipids 9, 10, 11 and 15 GFP RNA Lipid Nanoparticles (LNPs)

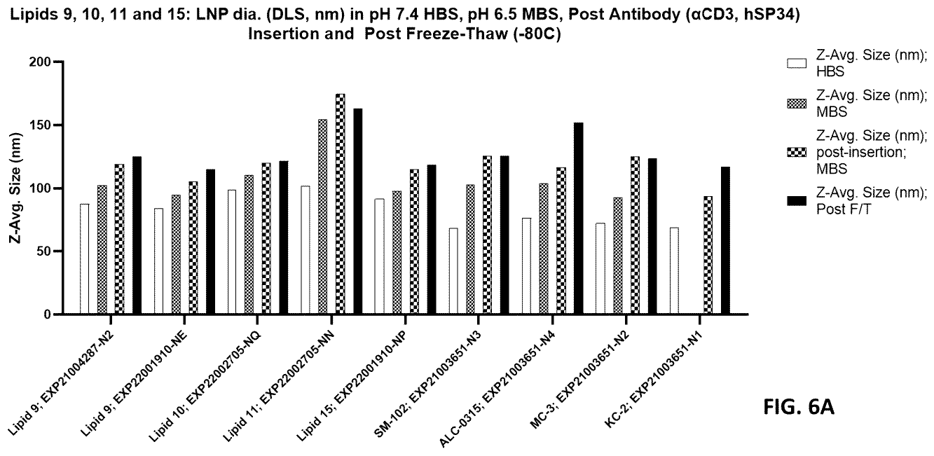

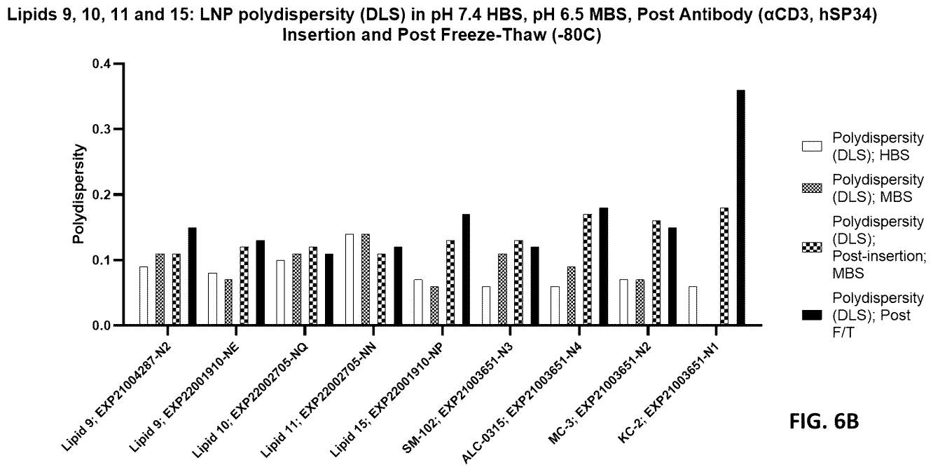

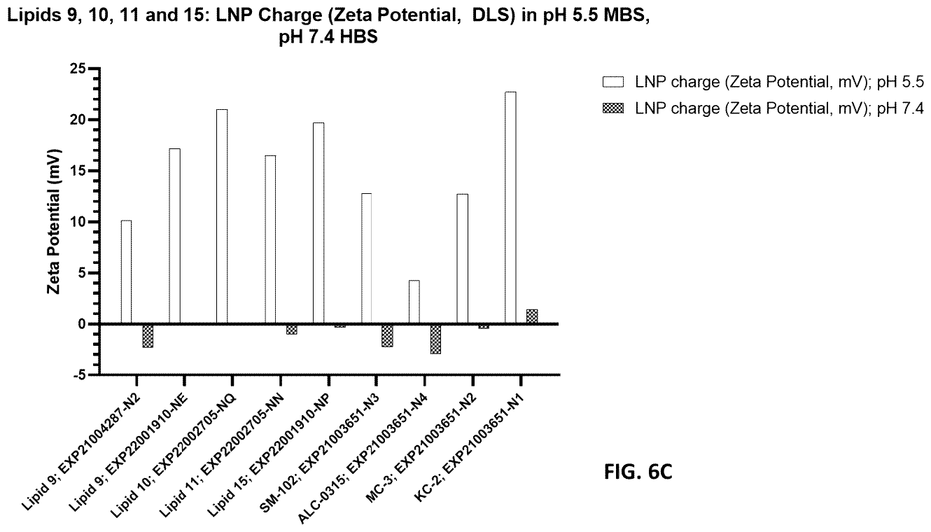

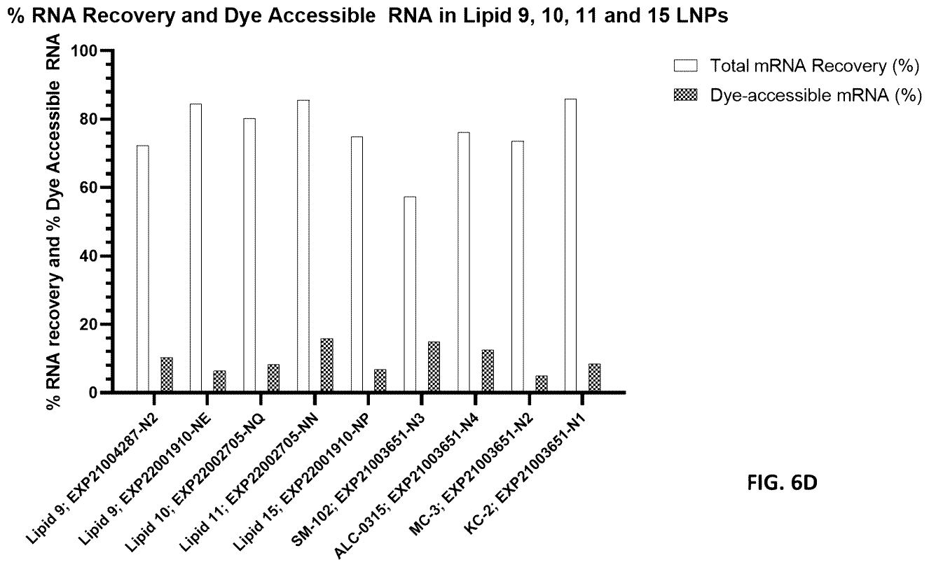

Lipids 9, 10, 11, and 15 LNPs encapsulating either GFP-mRNA (TriLink Biotechnologies Inc.) or CAR-RNA (custom made by TniLink Biotechnologies Inc.) were prepared and characterized using methods described in Examples 5 to 8. Measured LNP size, PDI, charge and RNA content values of resulting and of LNPs made using comparator ionizable lipids (including MC3, KC2, SM-102, and ALC-0315) are summarized in Table 13 to Table 15. As seen in FIG. 6A, the initial mixing step and subsequent buffer exchange into HIBS resulted LNP sizes ≤100 nm with Lipids 9, 10, 11 and 15. All lipids resulted in high encapsulation efficiency (<15% Dye accessible RNA) and >70% RNA recovery. The subsequent buffer exchange into pH 6.5 MES buffer and antibody insertion process (37C, 4 hours) was well tolerated by lipids 9, 10, and 15 LNPs resulting in final targeted LNP diameters below 140 nm and PDI<0.2. Lipid 11 LNPs exhibited the greatest size shift upon buffer exchange into pH 6.5 IS buffer, and a moderately larger size shift upon antibody insertion relative to the other lipids tested. All four tested lipid LNPs were stable to one freeze-thaw cycle with no significant shifts in LNP diameter or polydispersity observed. In summary, all four lipids tested resulted in viable mRNA encapsulation and freeze-thaw stability as well as <200 nm final targeted LNP diameters. The ability of lipids 9, 10, 11 and 15 LNPs for inducing in vitro protein transfection in primary human T-cells mediated by αCD3 or αCD8 T-cell receptor targeting was evaluated as described in Examples 16 and 17.

TABLE 13
Lipids 9, 10, 11, 15 LNPs and comparator Lipids LNP Size, Polydispersity (DLS) data
in pH 7.4 HBS, pH 6.5 MBS and post αCD3 antibody Fab (hSP34) conjugate insertion

| | Z-Avg. | Z-Avg. | Z-Avg. | Z-Avg. | | | PDI | |
|---|---|---|---|---|---|---|---|---|
| | Size | Size | Size (nm); | Size | PDI | PDI | (DLS); | PDI |
| Ionizable Lipid, | (nm); | (nm); | post-insertion; | (nm); | (DLS); | (DLS); | Post-insertion; | (DLS); |
| LNP no. | HBS | MBS | MBS | Post F/T | HBS | MBS | MBS | Post F/T |
| Lipid 9; | 87.6 | 102.4 | 118.9 | 125.3 | 0.09 | 0.11 | 0.11 | 0.15 |
| EXP21004287- | | | | | | | | |
| N2 | | | | | | | | |
| Lipid 9; | 83.9 | 94.8 | 105.1 | 115.1 | 0.08 | 0.07 | 0.12 | 0.13 |
| EXP22001910- | | | | | | | | |
| NE | | | | | | | | |
| Lipid 10; | 98.6 | 110.6 | 119.9 | 121.5 | 0.1 | 0.11 | 0.12 | 0.11 |
| EXP22002705- | | | | | | | | |
| NQ | | | | | | | | |
| Lipid 11; | 101.9 | 154.6 | 174.5 | 162.9 | 0.14 | 0.14 | 0.11 | 0.12 |
| EXP22002705- | | | | | | | | |
| NN | | | | | | | | |
| Lipid 15; | 91.55 | 97.65 | 114.8 | 118.3 | 0.07 | 0.06 | 0.13 | 0.17 |
| EXP22001910- | | | | | | | | |
| NP | | | | | | | | |
| SM-102; | 68.4 | 102.9 | 125.7 | 125.6 | 0.06 | 0.11 | 0.13 | 0.12 |
| EXP21003651- | | | | | | | | |
| N3 | | | | | | | | |
| ALC-0315; | 76.5 | 103.9 | 116.7 | 152.2 | 0.06 | 0.09 | 0.17 | 0.18 |
| EXP21003651- | | | | | | | | |
| N4 | | | | | | | | |
| DLin-MC3- | 72.2 | 92.6 | 124.8 | 123.65 | 0.07 | 0.07 | 0.16 | 0.15 |
| DMA; | | | | | | | | |
| EXP21003651- | | | | | | | | |
| N2 | | | | | | | | |
| DLin-KC2- | 68.8 | | 93.5 | 116.8 | 0.06 | | 0.18 | 0.36 |
| DMA; | | | | | | | | |
| EXP21003651- | | | | | | | | |
| N1 | | | | | | | | |

TABLE 14
Lipids 9, 10, 11, 15 Lipid and comparator Lipid LNP charge, Zeta
Potential, ZP (DLS, mV) in pH 5.5 MES and pH 7.4 HBS

| | LNP charge | LNP charge |
|---|---|---|
| | (ZP, mV); pH | (ZP, mV); pH |
| Ionizable Lipid, LNP no. | 5.5 | 7.4 |
| Lipid 9; EXP21004287-N2 | 10.1 | −2.34 |
| Lipid 9; EXP22001910-NE | 17.2 | 0.0437 |
| Lipid 10; EXP22002705-NQ | 21 | −0.137 |
| Lipid 11; EXP22002705-NN | 16.5 | −1.01 |
| Lipid 15; EXP22001910-NP | 19.7 | −0.322 |
| SM-102; EXP21003651-N3 | 12.8 | −2.25 |
| ALC-0315; EXP21003651-N4 | 4.25 | −2.95 |
| DLin-MC3-DMA; | 12.7 | −0.43 |
| EXP21003651-N2 | | |
| DLin-KC2-DMA; | 22.7 | 1.41 |
| EXP21003651-N1 | | |

TABLE 15
Lipids 9, 10, 11, 15 LNP and comparator Lipids LNP Dye Accessible RNA and
total RNA content

| | Nominal | | Dye- | Total | |
|---|---|---|---|---|---|
| | mRNA | Measured | accessible | mRNA | |
| Ionizable Lipid, LNP | Conc. | Total mRNA | mRNA | Recovery | Dye-accessible |
| no. | (μg/mL) | (μg/mL) | (μg/mL) | (%) | mRNA (%) |
| Lipid 9; EXP21004287- | 100 | 72.3 | 7.5 | 72.3 | 10.4 |
| N2 | | | | | |
| Lipid 9; EXP22001910- | 100 | 84.4 | 5.4 | 84.4 | 6.4 |
| NE | | | | | |
| Lipid 10; | 100 | 80.2 | 6.6 | 80.2 | 8.2 |
| EXP22002705-NQ | | | | | |
| Lipid 11; | 100 | 85.6 | 13.6 | 85.6 | 15.9 |
| EXP22002705-NN | | | | | |
| Lipid 15; | 100 | 74.9 | 5.1 | 74.9 | 6.8 |
| EXP22001910-NP | | | | | |

### Example 11. Physiochemical Properties (Pre- and Post-Insertion with αCD3 Fab Conjugate Hsp34) of Lipids 31-34 GFP RNA Lipid Nanoparticles (LNPs)

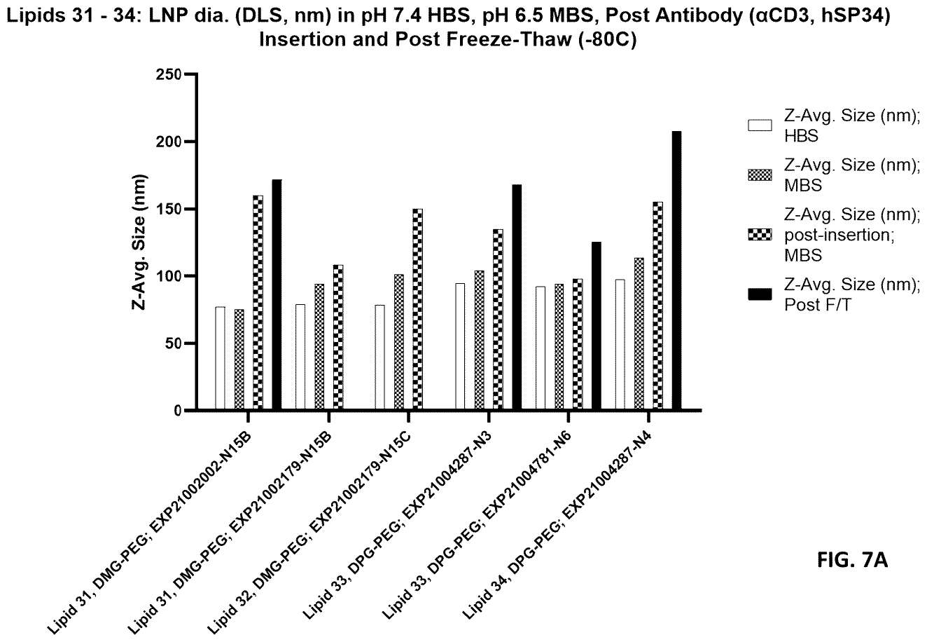

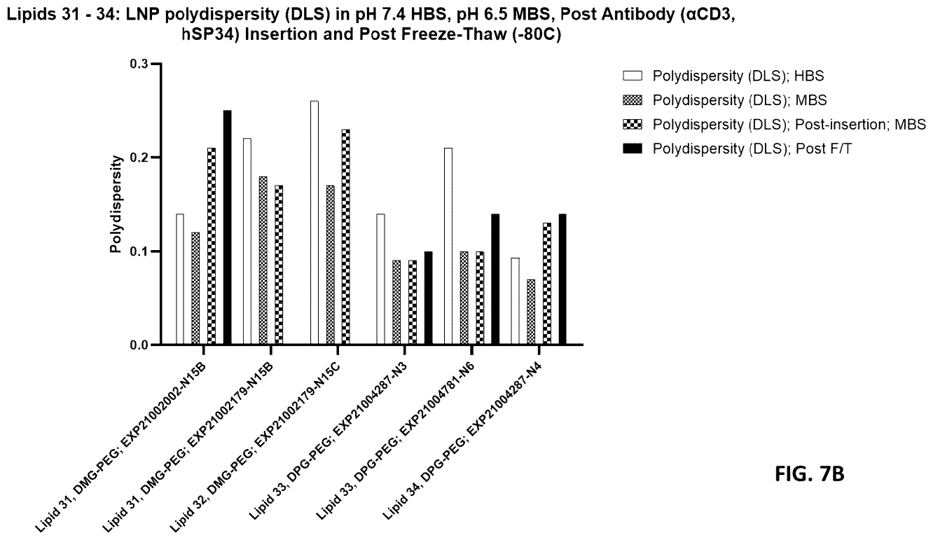

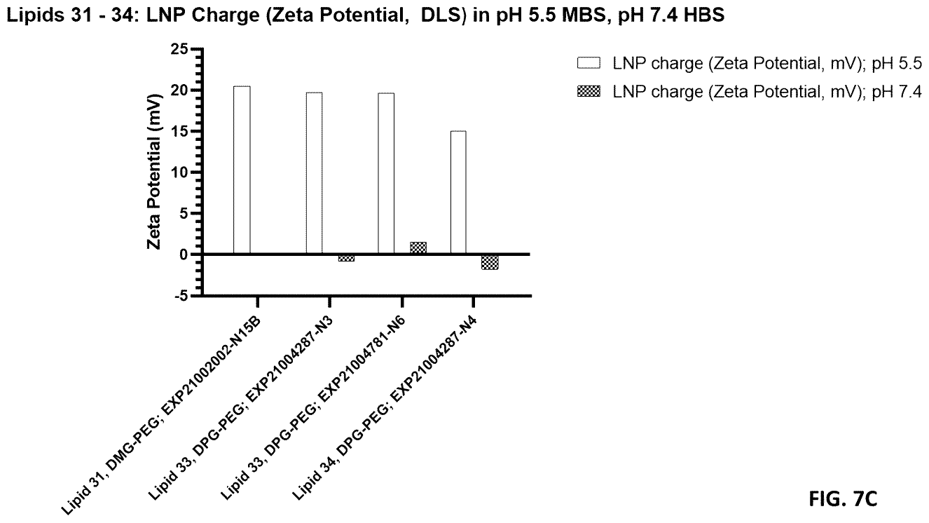

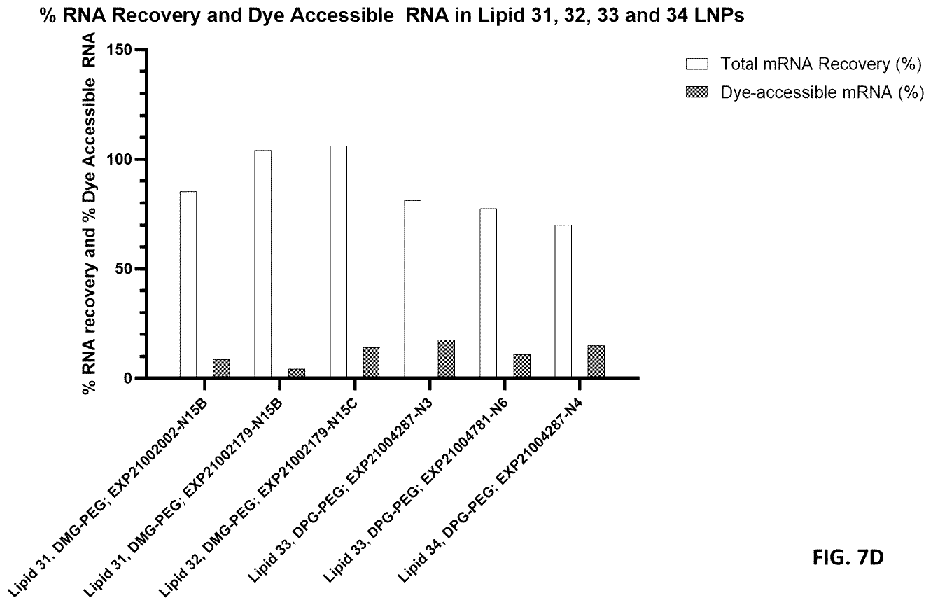

Lipids 31, 32, 33, and 34 LNPs encapsulating either GFP-mRNA (TriLink Biotechnologies Inc.) or CAR-RNA (custom made by TniLink Biotechnologies Inc.) were prepared and characterized using methods described in Examples 5 to 8. Measured LNP size, PDI, charge and RNA content values of Lipids 31, 32, 33, and 34 LNPs are summarized in Table 16 to Table 18. As seen in FIG. 7A, the initial mixing step and subsequent buffer exchange into THBS resulted in LNP sizes ≤110 nm with Lipids 31, 32, 33, and 34. All LNPs exhibited moderate to high encapsulation efficiency (<2000 Dye accessible RNA) and >7000 RNA recovery. The subsequent buffer exchange into pH 6.5 MES buffer and antibody insertion process (37C, 4 hours) resulted in final targeted LNP diameters in the 120 nm to 160 nm range and PDI in the 0.1-0.25 range indicating relatively poorer particle size control in lipids 31 through 34 LNPs. Lipid 31, 32, and 34 LNPs showed moderate size increases after one freeze-thaw cycle, however, a notably larger size shift was observed with lipid 34 LNPs. In summary, all four lipids tested resulted in viable mRNA encapsulation and freeze-thaw stability as well as <200 nm final targeted LNP diameters. The ability of lipids 31, 32, 33, 34 LNPs for inducing in vitro protein transfection in primary human T-cells mediated by αCD3 or αCD8 T-cell receptor targeting was evaluated as described in Examples 16 and 17.

TABLE 16
Lipids 31, 32, 33, 34 LNPs and comparator Lipids LNP Size, Polydispersity (DLS) data
in pH 7.4 HBS, pH 6.5 MBS and post αCD3 antibody Fab (hSP34) conjugate insertion

| | | | Z-Avg. | | | | | |
|---|---|---|---|---|---|---|---|---|
| | Z-Avg. | Z-Avg. | Size | Z-Avg. | | | Polydispersity | |
| Ionizable | Size | Size | (nm); | Size | Polydispersity | Polydispersity | (DLS); | Polydispersity |
| lipid, LNP | (nm); | (nm); | post-insertion; | (nm); | (DLS); | (DLS); | Post-insertion; | (DLS); |
| no. | HBS | MBS | MBS | Post F/T | HBS | MBS | MBS | Post F/T |
| Lipid 31, | 77.1 | 75.2 | 159.8 | 171.7 | 0.14 | 0.12 | 0.21 | 0.25 |
| DMG-PEG; | | | | | | | | |
| EXP21002002- | | | | | | | | |
| N15B | | | | | | | | |
| Lipid 31, | 79.23 | 94.3 | 108.3 | ** | 0.22 | 0.18 | 0.17 | |
| DMG-PEG; | | | | | | | | |
| EXP21002179- | | | | | | | | |
| N15B | | | | | | | | |
| Lipid 32, | 78.3 | 101.4 | 149.9 | ** | 0.26 | 0.17 | 0.23 | |
| DMG-PEG; | | | | | | | | |
| EXP21002179- | | | | | | | | |
| N15C | | | | | | | | |
| Lipid 33, | 94.74 | 103.9 | 134.8 | 167.8 | 0.14 | 0.09 | 0.09 | 0.1 |
| DPG-PEG; | | | | | | | | |
| EXP21004287- | | | | | | | | |
| N3 | | | | | | | | |
| Lipid 33, | 92.2 | 94.1 | 98.085 | 125.3 | 0.21 | 0.1 | 0.10 | 0.14 |
| DPG-PEG; | | | | | | | | |
| EXP21004781- | | | | | | | | |
| N6 | | | | | | | | |
| Lipid 34, | 97.31 | 113.3 | 155.1 | 207.5 | 0.093 | 0.07 | 0.13 | 0.14 |
| DPG-PEG; | | | | | | | | |
| EXP21004287- | | | | | | | | |
| N4 | | | | | | | | |

** Not measured.

TABLE 17
Lipids 31, 32, 33, 34 Lipid and comparator
Lipid LNP charge, Zeta Potential, ZP
(DLS, mV) in pH 5.5 MES and pH 7.4 HBS

| | Charge | Charge |
|---|---|---|
| | (ZP, mV); | (ZP, mV); |
| Ionizable lipid, LNP no. | pH 5.5 | pH 7.4 |
| Lipid 31, DMG-PEG; EXP21002002-N15B | 20.5 | −0.138 |
| Lipid 31, DMG-PEG; EXP21002179-N15B | | |
| Lipid 32, DMG-PEG; EXP21002179-N15C | | |
| Lipid 33, DPG-PEG; EXP21004287-N3 | 19.7 | −0.867 |
| Lipid 33, DPG-PEG; EXP21004781-N6 | 19.6 | 1.5 |
| Lipid 34, DPG-PEG; EXP21004287-N4 | 15 | −1.79 |

TABLE 18
Lipids 31, 32, 33, 34 LNP and comparator Lipids
LNP Dye Accessible RNA and total RNA content

| | Nominal | Measured | Dye- | Total | Dye- |
|---|---|---|---|---|---|
| | mRNA | Total | accessible | mRNA | accessible |
| | Conc. | mRNA | mRNA | Recovery | mRNA |
| Ionizable lipid, LNP no. | (μg/mL) | (μg/mL) | (μg/mL) | (%) | (%) |
| Lipid 31, DMG-PEG; | 50 | 42.7 | 3.7 | 85.4 | 8.7 |
| EXP21002002-N15B | | | | | |
| Lipid 31, DMG-PEG; | 50 | 52.13 | 2.2 | 104.26 | 4.2 |
| EXP21002179-N15B | | | | | |
| Lipid 32, DMG-PEG; | 50 | 53.02 | 7.5 | 106.04 | 14.1 |
| EXP21002179-N15C | | | | | |
| Lipid 33, DPG-PEG; | 100 | 81.2 | 14.3 | 81.2 | 17.6 |
| EXP21004287-N3 | | | | | |
| Lipid 33, DPG-PEG; | 150 | 116.4 | 12.8 | 77.6 | 110 |
| EXP21004781-N6 | | | | | |
| Lipid 34, DPG-PEG; | 100 | 70 | 10.5 | 70 | 15 |
| EXP21004287-N4 | | | | | |

### Example 12. Physiochemical Properties (Pre- and Post-Insertion with αCD8 Fab Conjugates TRX-2 and 15C01) of Lipids 1, 3, 4, 5, 9 and 15 GFP RNA Containing Lipid Nanoparticles (LNPs)

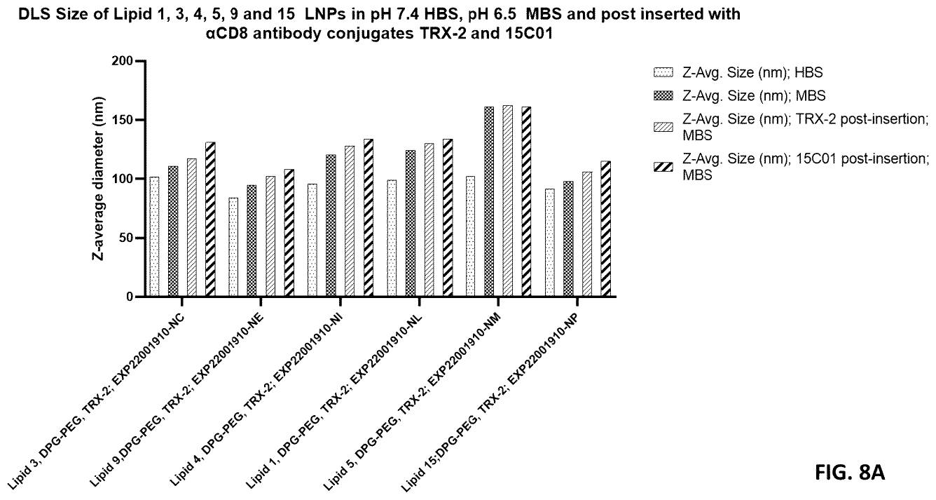

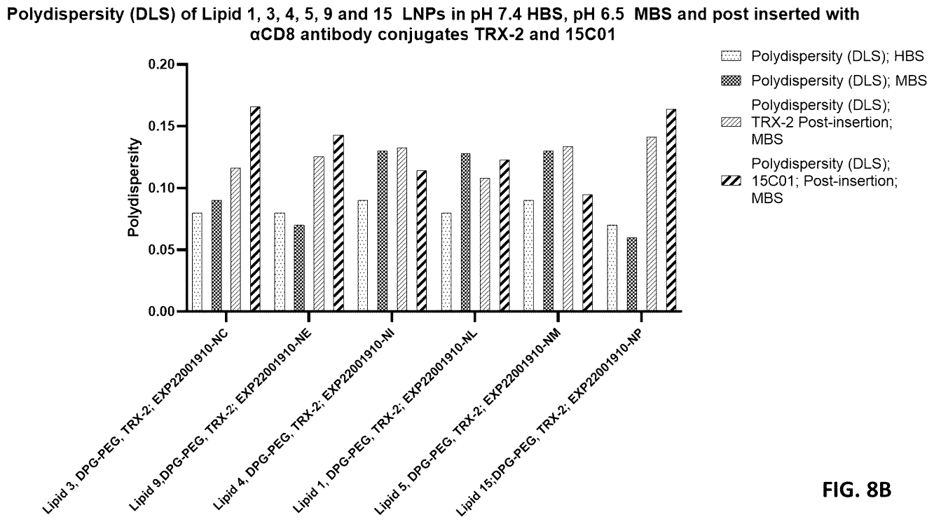

Lipids 1, 3, 4, 5, 9 and 15 LNPs encapsulating GFP-RNA (custom made by TriLink Biotechnologies Inc.) were prepared and characterized using methods described in Examples 5 to 8. Measured LNP size, and PDI, Lipids 1, 3, 4, 5, 9 and 15 LNPs in both pre- and post-insertion with αCD8 fab (TRX-1 and 15C01) conjugates are summarized in Table 19. As seen in FIG. 8A, the initial mixing step and subsequent buffer exchange into HBS resulted in LNP sizes ≤120 nm with Lipids 3, 9 and 15, while Lipid 5 LNPs exhibited >150 nm diameter upon buffer exchange into pH 6.5 MBS. Lipids 9 and 15 LNPs exhibited the smallest sizes (120 nm) after both αCD8 antibody conjugate insertions (TRX-2 and 15C01) and post one freeze-thaw cycle. Similarly polydispersity of all final (CD8 targeted LNPs remained (0.2 both pre- and post-freeze thaw (FIG. 8). The ability of lipids 1, 3, 4, 5, 9 and 15 LNPs for inducing in vitro protein transfection in primary human T-cells mediated by CD8 T-cell receptor targeting antibodies (TRX-2 and 15C01) was evaluated as described in Examples 18 and 19.

TABLE 19
Lipid 1, 3, 4, 5, 9, and 15 LNP size and PDI pre- and post-
insertion with αCD8 (TRX-1 and 15C01) targeting Fab conjugate

| | | | Z-Avg. | Z-Avg. | | | PDI | PDI |
|---|---|---|---|---|---|---|---|---|
| | | | Size (nm); | Size (nm); | | | (DLS); | (DLS); |
| | Z-Avg. | Z-Avg. | TRX-2 | 15C01 | | | TRX-2 | 15C01; |
| | Size | Size | post- | post- | PDI | PDI | Post- | Post- |
| Ionizable Lipid, | (nm); | (nm); | insertion; | insertion; | (DLS); | (DLS); | insertion; | insertion; |
| LNP no. | HBS | MBS | MBS | MBS | HBS | MBS | MBS | MBS |
| Lipid 3, DPG- | 101.5 | 110.8 | 117.4 | 131.0 | 0.08 | 0.09 | 0.116 | 0.166 |
| PEG, TRX-2; | | | | | | | | |
| EXP22001910- | | | | | | | | |
| NC | | | | | | | | |
| Lipid 9, DPG- | 83.9 | 94.8 | 102.5 | 108.1 | 0.08 | 0.07 | 0.1255 | 0.143 |
| PEG, TRX-2; | | | | | | | | |
| EXP22001910- | | | | | | | | |
| NE | | | | | | | | |
| Lipid 4, DPG- | 95.5 | 120.6 | 127.8 | 133.7 | 0.09 | 0.13 | 0.1325 | 0.114 |
| PEG, TRX-2; | | | | | | | | |
| EXP22001910- | | | | | | | | |
| NI | | | | | | | | |
| Lipid 1, DPG- | 98.78 | 124.25 | 130.2 | 133.7 | 0.08 | 0.128 | 0.108 | 0.123 |
| PEG, TRX-2; | | | | | | | | |
| EXP22001910- | | | | | | | | |
| NL | | | | | | | | |
| Lipid 5, DPG- | 102.4 | 161.2 | 162.3 | 161.3 | 0.09 | 0.13 | 0.1335 | 0.095 |
| PEG, TRX-2; | | | | | | | | |
| EXP22001910- | | | | | | | | |
| NM | | | | | | | | |
| Lipid 15; DPG- | 91.55 | 97.65 | 106.0 | 115.1 | 0.07 | 0.06 | 0.1415 | 0.164 |
| PEG, TRX-2; | | | | | | | | |
| EXP22001910- | | | | | | | | |
| NP | | | | | | | | |

### Example 13. Physiochemical Properties (Pre- and Post-Insertion With αCD8 (TRX-2) Far Conjugate) of Lipids 1, 8, 9, 10, 11 and 15 GFP RNA Containing Lipid Nanoparticles (LNPs)

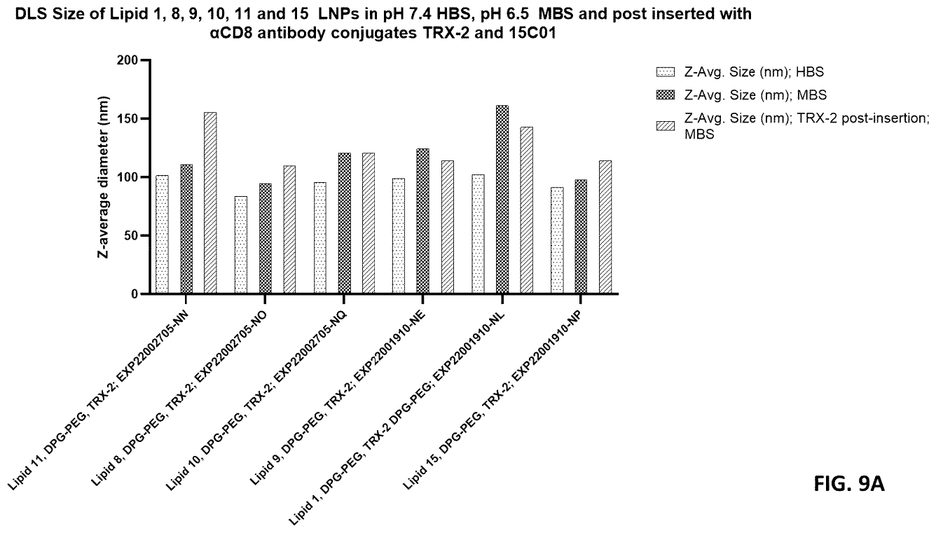

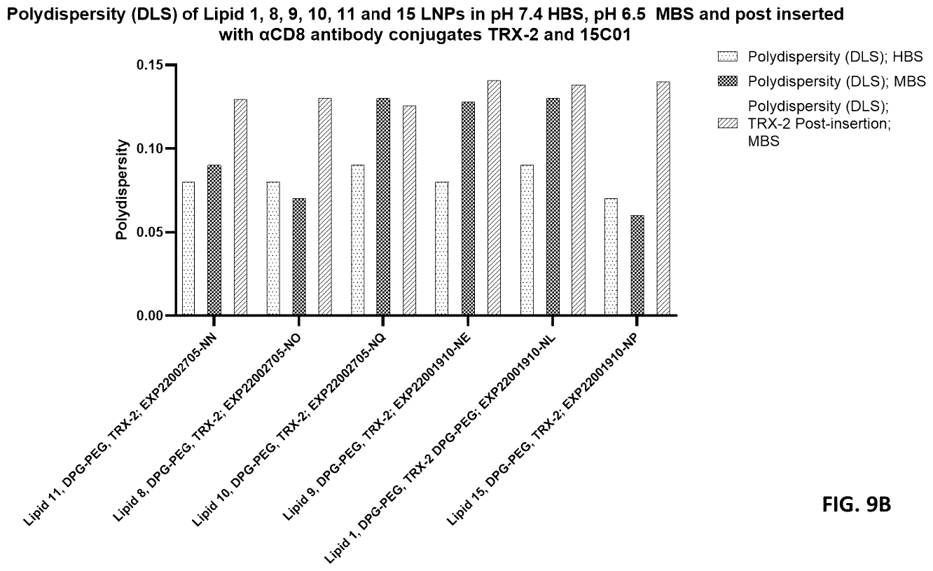

Lipids 1, 8, 9, 10, 11 and 15 LNPs encapsulating GFP-RNA (custom made by TniLink Biotechnologies Inc.) were prepared and characterized using methods described in Examples 5 to 8. Measured LNP size, and PDI, Lipids 1, 8, 9, 10, 11 and 15 LNPs in both pre- and post-insertion with αCD8 (TRX-2) Fab conjugate are summarized in Table 20. As seen in FIG. 9A, the initial mixing step and subsequent buffer exchange into HIBS resulted in LNP sizes <130 nm with Lipids 8, 9, 10, 11 and 15, while Lipid 1 LNPs exhibited >150 nm diameter upon buffer exchange into pH 6.5 MBS. Lipids 8, 9, 10 and 15 LNPs exhibited the smallest sizes (≤130 nm) after both αCD8 antibody conjugate insertions (TRX-2 and 15C01) and post one freeze-thaw cycle. Polydispersity of all final αCD8 targeted LNPs remained <0.2 both pre- and post-freeze thaw (FIG. 9B). The ability of lipids 1, 8, 9, 10, 11 and 15 LNPs for inducing in vitro protein transfection in primary human T-cells mediated by αCD8 T-cell receptor targeting antibodies (TRX-2 and 15C01) was evaluated as described in Examples 16 and 17.

TABLE 20
Lipid 1, 8, 9, 10, 11 and 15 LNP size and PDI pre- and post-
insertion with αCD8 (TRX-1) targeting Fab conjugate

| | Z-Avg. | Z-Avg. | Z-Avg. Size | | | PDI (DLS); |
|---|---|---|---|---|---|---|
| | Size | Size | (nm); TRX-2 | PDI | PDI | TRX-2 Post- |
| | (nm); | (nm); | post-insertion; | (DLS); | (DLS); | insertion; |
| Ionizable Lipid, LNP no. | HBS | MBS | MBS | HBS | MBS | MBS |
| Lipid 11, DPG-PEG, | 101.5 | 110.8 | 155.45 | 0.08 | 0.09 | 0.13 |
| TRX-2; EXP22002705- | | | | | | |
| NN | | | | | | |
| Lipid 8, DPG-PEG, | 83.9 | 94.8 | 110 | 0.08 | 0.07 | 0.13 |
| TRX-2; EXP22002705- | | | | | | |
| NO | | | | | | |
| Lipid 10, DPG-PEG, | 95.5 | 120.6 | 120.65 | 0.09 | 0.13 | 0.13 |
| TRX-2; EXP22002705- | | | | | | |
| NQ | | | | | | |
| Lipid 9, DPG-PEG, | 98.78 | 124.25 | 114.45 | 0.08 | 0.128 | 0.14 |
| TRX-2; EXP22001910- | | | | | | |
| NE | | | | | | |
| Lipid 1, DPG-PEG, | 102.4 | 161.2 | 142.75 | 0.09 | 0.13 | 0.14 |
| TRX-2 DPG-PEG; | | | | | | |
| EXP22001910-NL | | | | | | |
| Lipid 15, DPG-PEG, | 91.55 | 97.65 | 114.45 | 0.07 | 0.06 | 0.14 |
| TRX-2; EXP22001910- | | | | | | |
| NP | | | | | | |

### Example 14. Physiochemical Properties (Pre- and Post-Insertion with aCD8 Fab Conjugate T8) of Lipids 3, 4, 33, and 34 CAR (TTR-023) RNA Lipid Nanoparticles (LNPS)

Physiochemical properties (pre- and post-insertion with αCD8 Fab conjugate T8) of Lipids 3, 4, 33, and 34 CAR (TTR-023) RNA Lipid nanoparticles (LNPs)

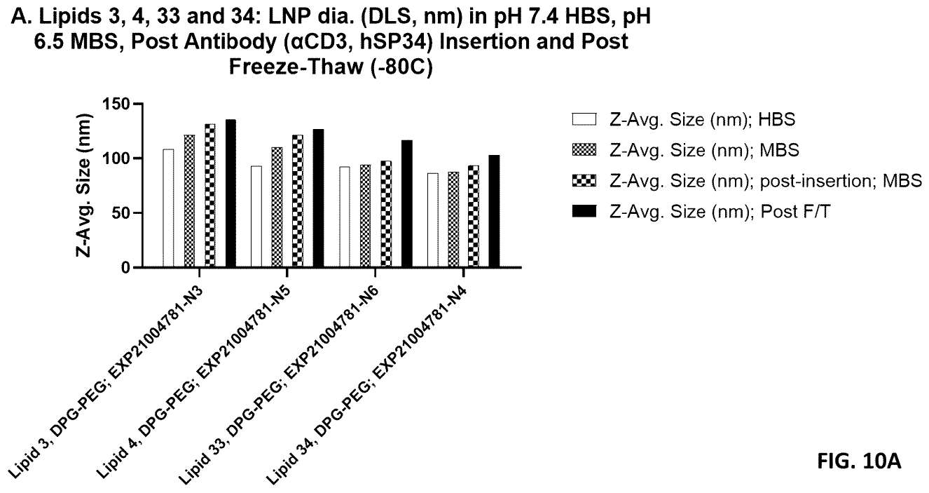

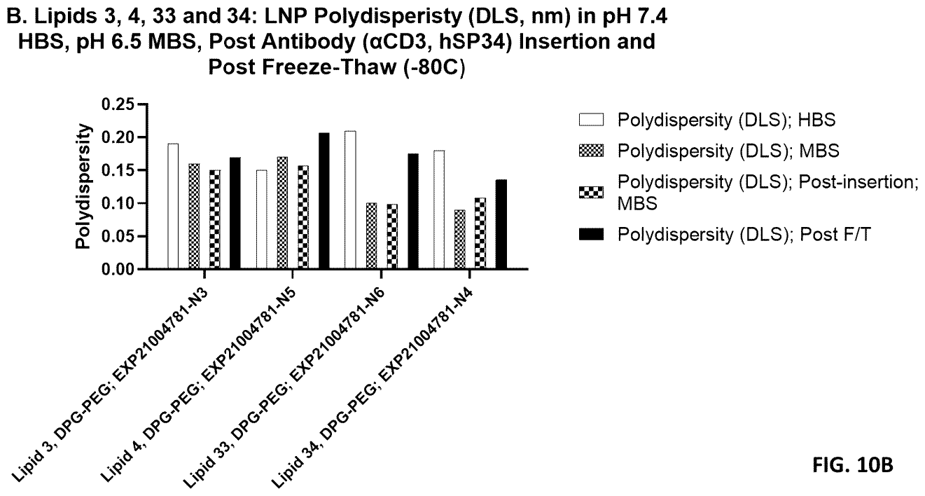

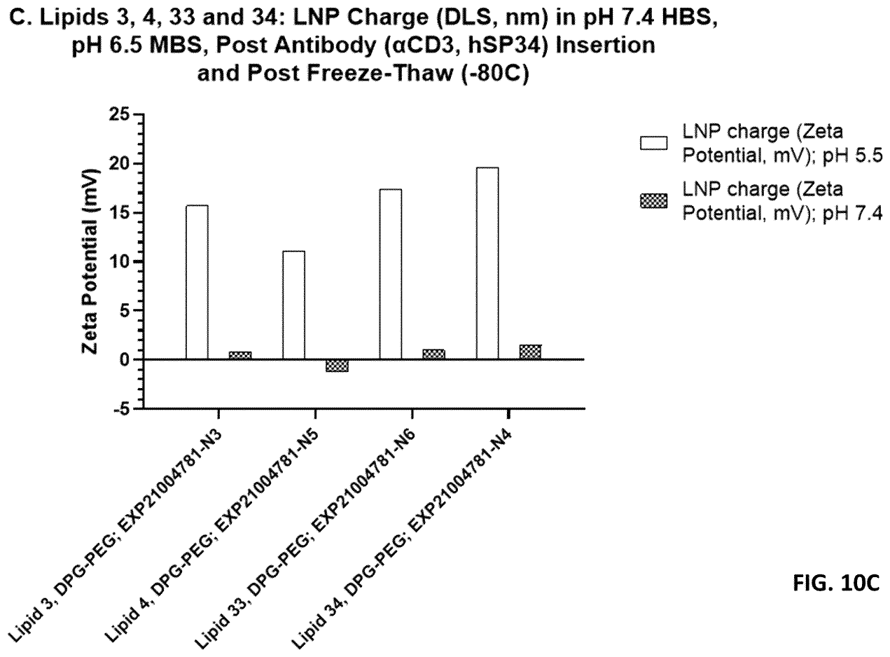

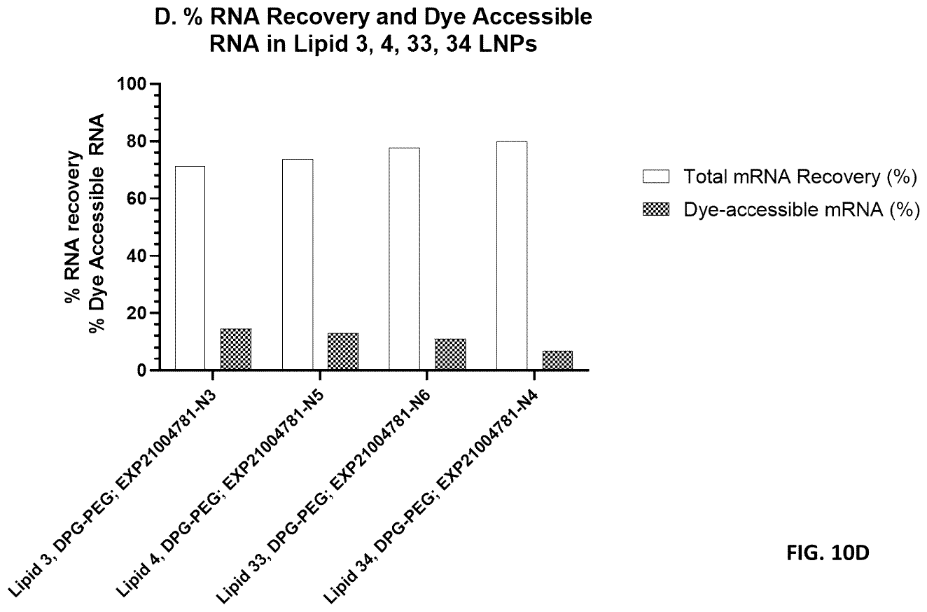

Lipids 3, 4, 33, and 34 LNPs encapsulating CAR (TTR-023) RNA (custom made by TriLink Biotechnologies Inc.) were prepared and characterized using methods described in Examples 5 to 8. Measured LNP size, PDI, charge and RNA content values of Lipids 3, 4, 33, and 34 LNPs are summarized in Table 21, Table 22 and Table 23. As seen in FIG. 10A, the initial mixing step and subsequent buffer exchanges into HBS and then MBS resulted in LNP sizes ≤125 nm with Lipids 3 and 4, and ≤100 nm with Lipids 33 and 34. All LNPs exhibited moderate to high encapsulation efficiency (<15% Dye accessible RNA) and >70% RNA recovery, with Lipids 33 and 34 trending better in terms of recovery and lower in terms of dye accessible RNA (FIG. 10D). The subsequent buffer exchange into pH 6.5 MES buffer and antibody insertion process (37C, 4 hours) resulted in Lipid 3 and 4 LNP diameters in the 120 nm to 135 nm range, while Lipids 33 and 34 yield sub-100 nm diameters. Similarly, Lipid 3 and 4 LNP PDI trended slightly higher in the 0.15 to 0.21 range, while Lipids 33 and 34 yield PDI ≤0.11, indicating slightly better size distribution properties. Lipid 4 and 34 LNPs showed moderate size increases after one freeze-thaw cycle, however, a notably larger size shifts were observed with lipid 3 and 33 LNPs. In summary, all four lipids tested resulted in viable CAR mRNA encapsulation and freeze-thaw stability as well as <150 nm final targeted LNP diameters. The ability of lipids 3, 4, 33, and 34 LNPs for inducing in vitro CAR protein expression in primary human T-cells mediated by αCD3 or αCD8 T-cell receptor targeting was evaluated as described in Examples 16 and 17.

TABLE 21
Lipids 3, 4, 33, 34 LNPs Size, Polydispersity (DLS) data in pH 7.4 HBS, pH 6.5
MBS and post αCD3 antibody Fab (hSP34) conjugate insertion

| | | | Z-Avg. | Z-Avg. | | | Polydispersity | |
|---|---|---|---|---|---|---|---|---|
| | Z-Avg. | Z-Avg. | Size (nm); | Size | | | (DLS); | |
| Ionizable | Size | Size | post- | (nm); | Polydispersity | Polydispersity | Post- | Polydispersity |
| lipid, LNP | (nm); | (nm); | insertion; | Post | (DLS); | (DLS); | insertion; | (DLS); |
| no. | HBS | MBS | MBS | F/T | HBS | MBS | MBS | Post F/T |
| Lipid 3, | 108.6 | 121.5 | 131.8 | 135.5 | 0.19 | 0.16 | 0.15 | 0.17 |
| DPG-PEG; | | | | | | | | |
| EXP21004781- | | | | | | | | |
| N3 | | | | | | | | |
| Lipid 4, | 92.8 | 110.2 | 121.7 | 127.1 | 0.15 | 0.17 | 0.16 | 0.21 |
| DPG-PEG; | | | | | | | | |
| EXP21004781- | | | | | | | | |
| N5 | | | | | | | | |
| Lipid 33, | 92.2 | 94.1 | 97.5 | 116.9 | 0.21 | 0.1 | 0.10 | 0.18 |
| DPG-PEG; | | | | | | | | |
| EXP21004781- | | | | | | | | |
| N6 | | | | | | | | |
| Lipid 34, | 86.3 | 87.8 | 93.4 | 103 | 0.18 | 0.09 | 0.11 | 0.14 |
| DPG-PEG; | | | | | | | | |
| EXP21004781- | | | | | | | | |
| N4 | | | | | | | | |

TABLE 22
Lipids 3, 4, 33, 34 LNP charge, Zeta Potential, ZP (DLS, mV) in
pH 5.5 MES and pH 7.4 HBS

| | Charge (ZP, | Charge (ZP, |
|---|---|---|
| | mV); | mV); |
| Ionizable lipid, LNP no. | pH 5.5 | pH 7.4 |
| Lipid 3, DPG-PEG; EXP21004781-N3 | 15.7 | 0.786 |
| Lipid 4, DPG-PEG; EXP21004781-N5 | 11.1 | −1.23 |
| Lipid 33, DPG-PEG; EXP21004781-N6 | 17.4 | 1.01 |
| Lipid 34, DPG-PEG; EXP21004781-N4 | 19.6 | 1.5 |

TABLE 23
Lipids 3, 4, 33, 34 LNP Dye Accessible RNA and total RNA content

| | Nominal | Measured | Dye- | Total | Dye- |
|---|---|---|---|---|---|
| Ionizable | mRNA | Total | accessible | mRNA | accessible |
| lipid, | Conc. | mRNA | mRNA | Recovery | mRNA |
| LNP no. | (μg/mL) | (μg/mL) | (μg/mL) | (%) | (%) |
| Lipid 3, | 150 | 107.1 | 15.5 | 71.4 | 14.5 |
| DPG-PEG; | | | | | |
| EXP21004781- | | | | | |
| N3 | | | | | |
| Lipid 4, | 150 | 110.7 | 14.4 | 73.8 | 13.0 |
| DPG-PEG; | | | | | |
| EXP21004781- | | | | | |
| N5 | | | | | |
| Lipid 33, | 150 | 116.4 | 12.8 | 77.6 | 11.0 |
| DPG-PEG; | | | | | |
| EXP21004781- | | | | | |
| N6 | | | | | |
| Lipid 34, | 150 | 119.9 | 8.2 | 79.9 | 6.8 |
| DPG-PEG; | | | | | |
| EXP21004781- | | | | | |
| N4 | | | | | |

### Example 15. Method for Freezing (and Thaw) Process for LNP Suspension and LNP Characterization Post Freeze-Thaw

LNP suspension was mixed with a solution of 49 wt % sucrose solution in water and additional storage buffer (if needed) to achieve a final sample containing LNPs at approximately 45 μg/mL and sucrose at approximately 9.6 wt %. Aliquots of approximately 0.05 mL in 1.5 mL centrifuge tubes were then prepared from the final LNP sample containing sucrose. The aliquots were then placed in a −80° C. freezer for at least 2 h to freeze the samples. After freezing, an aliquot was thawed by placing it at room temperature for at least 10 min. The aliquot was then mixed by vortexing at 2500 rpm for approximately 5 s. The thawed material was then analyzed for size by DLS as described in Example 3.

### Example 16. Method for the Primary Human T-Cell Transfection with DiI-C18-5DS Labelled LNPs

CD3+ T cells were isolated from frozen peripheral blood mononuclear cells using an EasySep Human T Cell Isolation Kit on a RoboSep automated cell isolation system from STEMCELL. T cells were plated into a round bottom 96-well plate in RPMI cell culture media supplemented with glutamax, 10% fetal bovine serum, pen-strep, and 40 ng/mL IL-2. 100 μL of cell suspension was seeded per well at a density of 1M T cells/mL (100K T cells/well). Cells were allowed to rest for two hours in a 37° C. incubator, and then were transfected by gently adding 10 μL of a 22 μg/mL (by mRNA) nanoparticle suspension, resulting in a final mRNA concentration of 2 μg/mL (unless otherwise noted). Cells were gently mixed with a pipette and then incubated for 24 hours in a 37° C. incubator. After incubation the cells were diluted with FACS buffer (BD 554657) and analyzed using a BD Fortessa flow cytometer. Data were analyzed using FlowJo software from BD biosciences.

### Example 17. CD4 and CD69 Staining Protocol Post LNP Transfection

After 24 hours, cells were transferred to a 96-well conical bottom polypropylene plate and centrifuged at 350×g for 5 minutes. Supernatants were removed and transferred to a fresh conical bottom polypropylene plate for further analysis. Cells were washed by adding 200 μL FACS buffer (BD 554657), centrifuging at 350×g for 5 min, and then aspirating the supernatant from each well. BV421 anti-human CD69 (BioLegend 310930 clone FN50) and BV711 anti-human CD4 (BioLegend 344648 clone SK3) antibodies were diluted 100× by adding 100 μL of each antibody to 10 mL FACS buffer. 100 μL of the diluted antibody solution was added to each well and the plate was incubated at room temperature for 20 minutes. The plates were then washed by centrifuging at 350×g for 5 min, removing the supernatant, re-suspending in 200 μL FACS buffer, centrifuging at 350×g for 5 min and aspirating the supernatant from each well. Following the wash, cells were resuspended in 100 μL of 1.6% formaldehyde and stored at 4° C. until FACS analysis. FACS analysis was performed using a BD Fortessa equipped with a High Throughput Sampler.

### Example 18. Human IFN-τ ELISA Method

IFN-γ was assayed using an R&D Duoset TL-2 ELISA kit, PN DY285B. Briefly: an Immulon 2HB 96-well plate (Thermo X1506319) was coated by adding 100 μL of a 2 μg/mL solution of the R&D IL-2 capture antibody to each well and then incubating the plate overnight at 4° C. The plate was washed three times with wash buffer (0.05 TWEEN-20 in pH 7.4 TRIS buffered saline, Thermo 28360), blocked with reagent diluent (0.1% BSA in wash buffer) for one hour at room temperature, and then washed an additional three times with wash buffer. Supernatants were diluted three-fold in reagent diluent and then 100 μL of diluted supernatant was added to each well. IFN-γ standards were prepared on the same plate by serial dilution. Plates were incubated for two hours at room temperature, washed three times with wash buffer. 100 μL of detection antibody diluted in reagent diluent was added, incubated for 2 hours at room temperature, and then the plate was washed three times with wash buffer. 100 μL of Streptavidin-HRP was added, incubated for 20 minutes at room temperature, and then the plate was washed three times with wash buffer. 100 μL of substrate solution (Thermo N301) was added, incubated for 20 minutes at room temperature and then the reaction was quenched by adding 100 μL of stop solution (Invitrogen SS04). Optical density at 450 nm was read on a SpectraMax M5 plate reader. IFN-γ concentration was quantified relative to a standard curve based on contemporaneously analyzed IFN-γ standards.

### Example 19. TTR-023 CAR (M1) Staining Protocol

Cells were transferred to a 96-well conical bottom plate and washed by centrifuging at 350×g for 5 minutes followed by resuspension in FACS buffer (BD 554657). TTR-023 CAR protein, which contains a FLAG tag, was detected by staining with M1 anti-Flag antibody (Sigma Aldrich F3040) conjugated to Alexa Fluor 488 (Invitrogen A3750) at room temperature for 20 minutes. Following staining, cells were washed twice by centrifugation at 350×g for 5 minutes followed by resuspension in FACS buffer. Cells were analyzed by FACS using a BD Fortessa flow cytometer (BD Biosciences).

Synthesis and purification procedure for the M1 anti-Flag antibody—Alexa Fluor 488 conjugate: M1 anti-FLAG antibody was buffer exchanged into pH 8.3 sodium bicarbonate buffer using a Zeba 40K MWCO spin column. An 8 mg/mL solution of Alexa Fluor 488 TFP ester (Invitrogen A37570) in DMSO was then added to a final AB:dye mass ratio of 10:1 and the mixture was allowed to react for 1 hour with gentle shaking. Fluorophore conjugated M1 was purified and buffer exchanged into pH 7.4 PBS by passing the reaction mixture through two sequential Zeba spin columns. Protein content and degree of labeling were assessed by UV-Vis spectroscopy.

### Example 20. In Vitro LNP Car Primary Human T-Cell Transfection and Raji (B-Cell) Co-Culture for Assessment of T-Cell Function

CD3+ or CD8+ T cells were isolated from human PBMCs by using EasySep Human CD8+ T cell isolation kit (catalog #17953) and EasySep Human T cell isolation kit (catalog #17951) according to manufacturer's instructions. Isolated T cells were rested overnight in T cell media (RPMI+Glutamax, Gibco, catalog #61870-036) with 10% heat inactivated fetal bovine serum (HI-FBS, Gibco, catalog #16140-071) supplemented with 100 IU human IL-2 (Miltenyi catalog #130-097-748) at a density of 500,000 cells/well in a 96 well plate. T cells were then transfected with LNPs at a mRNA concentration of 2 μg/ml. CAR expression was detected 18 h post LNP transfection by using flow cytometry. Before co-culture with Raji B-cells, the transfected T cells were washed twice with T cell media. Raji B-cells were stained with cell trace violet (CTV, Thermo Fisher, catalog #C34571) and co-cultured with different effector to target (E:T) ratios of T cells in a 96 well plate format and incubated at 37° C. for 72 hours. 72 hours post co-culture the cells were stained with live/dead stain (eBioscience Fixable viability dye eFluor 780, Invitrogen catalog #2290917) and analyzed by flow cytometry. Data was analyzed with FlowJo and Graph Pad prism.

### Example 21. In Vitro Experimental Protocol for Whole Blood Transfection

This Example describes the method used to transfect immune cells in whole blood using Fab targeted mRNA LNPs.

Venous blood from healthy volunteers was anti-coagulated in heparin tubes (BD Biosciences #367526) and seeded at 50 μL in a 96-well round-bottom plate. Transfection of whole blood was carried out simply by adding nanoparticles containing 5 μg/mL mRNA to the cells and co-culturing at 37° C. until the time of analysis. To assess transfection efficiency, cells were analyzed 24-hours post-transfection by flow cytometry. LNPs used (with and without post-inserted targets) at 2.5 μg/mL:RDM073.23. Cells obtained from human blood were analyzed by flow cytometry. Prior to the analysis of whole blood transfection efficiency, red blood cells were lysed twice with VersaLyse Lysing Solution (Beckman Coulter #A09777) for 10 minutes at room temperature. Primary antibodies applied in the flow cytometry analysis of whole blood included the following: CD4-FITC (1:200) (BD Biosiences #555346), CD19-BUV395 (1:400) (BD Biosiences #563551), CD56-BUV737 (1:400) (BD Biosiences #741842). Fixable Viability Dye eFluor780 (eBiosciences #65-0865-14) was used to assess viability for all samples. For flow analysis, 1×10⁵ cells were Fc-blocked (BD Biosciences #564219) for 5 minutes on ice, followed by labeling dead cells with fixable viability dye eFluor780 and surface staining for 30 minutes on ice with specific antibodies.

Compensation for each fluorochrome was performed in the multicolor flow panels using positive and negative compensation beads. Fluorescence minus one (FMO) samples and unstained controls were included to determine the level of background fluorescence and to set the gates for the negative cell populations versus the positive cell populations.

All samples were acquired on a BD LSRFortessa X-20 (BD Biosciences) running FACSDIVA software (Becton Dickinson). All data collected were analyzed using FlowJo 10.7.1 software and GraphPad Prism version 9.0.

### Example 22. Protocol for In Vivo T-Cell Reprogramming (Using GFP Reporter Protein) in Human T-Cell Engrafted NSG-Mice

The following is a standard procedure for in-vivo reprogramming of immune cells with DiI LNP expressing GFP.

### Mice Strains and Humanization

The NSG (NOD.Cg-Prkdcscid Il2rgtm1Wjl/SzJ) mouse model was purchased from Jax Laboratories. 6-8 weeks old male mice were engrafted with 10 million PBMC of qualified donor in sterile PBS by tail vein injection. Individual body weight was monitored twice a week and blood samples were collected at appropriate interval to evaluate human immune cells engraftment.

### Evaluation of Human T-Cell Engraftment in the Immunodeficient Mice

50 ul blood was collected by tail vein bleed from each mouse. Red blood cells were lysed using Versalyse, RBC lysis solution following protocol as instructed by manufacturer (Beckman Coulter A09777). Cells were stained with hCD45 & hCD3 to determine the engraftment of human T-cells. After 15 days of PBMC injection, mice had anywhere from 30-60% huCD45+. These humanized mice were evaluated for reprogramming of immune cells by LNPs expressing DiI dye and GFP.

### Reprogramming of Immune Cells

At time zero, mice (n=4 per group) were injected with GFP expressing DiI LNPs (by i.v. with appropriate buffer). At each time point, 24 or 48 h depending on the example mice treated with either LNPs or buffer were sacrificed. Terminal blood and tissues collection was performed to determine DiI and GFP expression in different organs and immune cells as below.

### Tissue and Blood Sample Collection.

At above specified timepoints, mice were anesthetized with CO₂ before sample collection. For blood collection, the chest was opened to expose the heart. Up to 300 μl blood was drawn from the left ventricle and dispensed into a K₃EDTA mini collect tube (Greiner Bio-One). Then a new syringe was used to draw remaining blood from the heart as much as possible. All the immune organs; spleen, bone marrow was isolated along with liver and lung. Immune cells were isolated from spleen, via smearing and shredding it through syringe and cell suspension was filtered through 70 μM cell strainer and was washed with PBS. Bone marrow was flushed with needle to collect all the immune cells. A piece of liver and lung tissue was gently grinded with tissue homogenizer and the homogenized and cells were isolated using militenyi liver dissociation kit, (Miltenyi Biotec, Catalog #130-105-807) and lung dissociation kit (Miltenyi Biotec, Catalog #130-0950927) and instruction were followed according to the manufacturing instruction.

### Immunophenotyping Analysis

Immune cells from blood and all the above organs were processed with Versalyse, RBC lysis buffer as per manufacturing instructions. Immune cells were stained with live/dead fixable dye and surface markers with standard flow analysis protocol as shown in Table 24. BD symphony flow cytometer was used to determine positive population.

TABLE 24

| Antigen | Fluorophore | Clone | Company | Catalog# |
|---|---|---|---|---|
| DiI | APC | NA | NA | NA |
| GFP | mRNA | NA | NA | NA |
| Anti-CD45 | BUV395 | HI30 | BD Biosciences | 563792 |
| Anti-CD3 | BUV805 | UCHT1 | BD Biosciences | 612895 |
| Anti-CD4 | BV711 | SK3 | BioLegend | 344648 |
| Anti-CD8 | BV421 | RPA-TB | BD Biosciences | 562428 |
| Anti-CD45 | BB700 | 30-F11 | BD Biosciences | 566439 |
| Anti-CD11b | BV785 | M1/70 | BioLegend | 101243 |
| Anti-F4/80 | PE Dazzle | BM8 | BioLegend | 123146 |
| Anti-CD31 | BUV737 | MEC 13.3 | BD Biosciences | 612802 |
| TruStain | NA | NA | BioLegend | 462103 |
| Monocyte | | | | |
| Blocker | | | | |
| Arc Amine | NA | NA | NA | 01-3333-42 |
| Comp beads | | | | |
| UltraComp | NA | NA | Invitrogen | 01-3333-42 |
| eBeads | | | | |
| LIVE/DEAD | NA | NA | Invitrogen | L34974 |
| Far Red Stain | | | | |
| TruStain Fc X | NA | NA | Invitrogen | 422302 |

### Example 23. In Vitro Protein Expression (GFP) in Primary Human T-Cells of Lipids 3, 6, 7 and Comparator Lipid aCD3 Targeted LNPs (DLin-MC3-DMA and ALC-0315) Stored at 4° C. and after One Freeze-Thaw Cycle (Post −80° C. Storage)

This example compares the GFP protein expression resulting from LNP's derived from Lipids 3, 6 and 7 to LNPs made using comparator lipids DLin-MC3-DMA and ALC-0315. Nanoparticles bearing GFP encoding mRNA (and optionally a fluorescent dye label (DiI-C18-5DS)) were produced using the microfluidic mixing and buffer exchange processes described in Example 2. An αCD3 Fab-conjugate was incorporated into the parent LNPs to obtain the final Antibody targeted LNP formulation using the process described in Example 5. Particles thus produced were tested in vitro in primary human CD3+ T cells to assess reported gene expression.

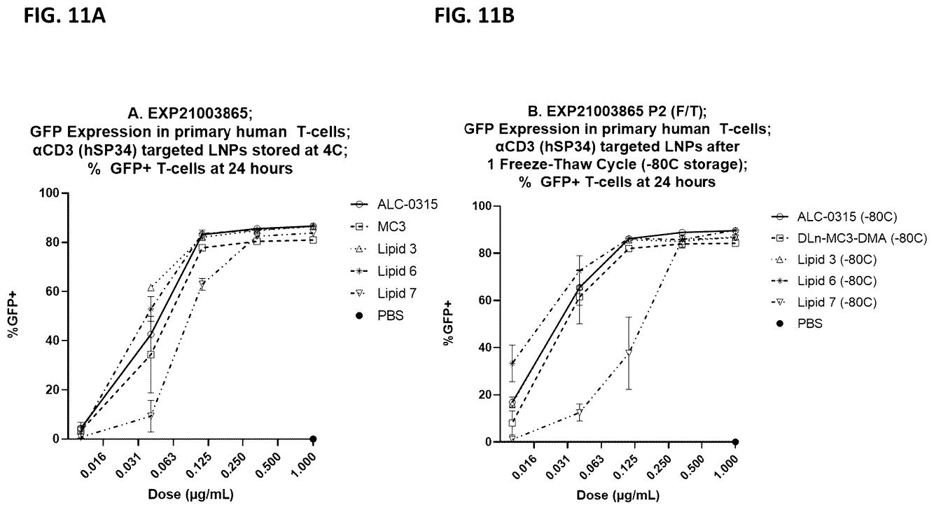

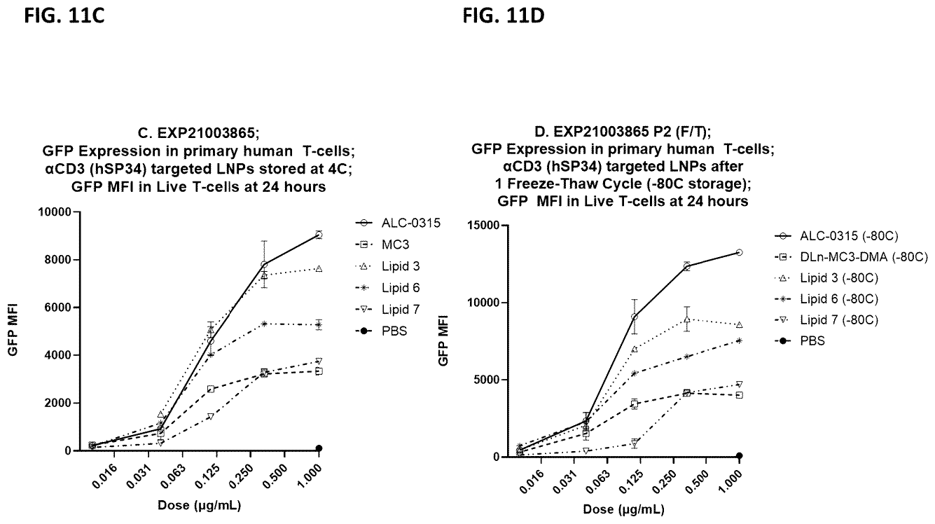

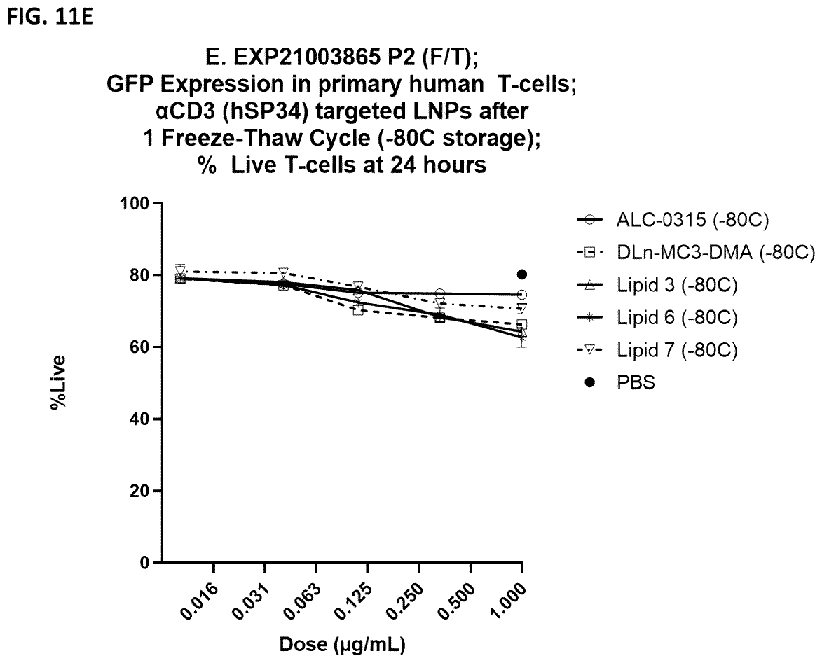

Lipid 3 and ALC-0315 LNPs resulted in similar GFP protein expression levels as indicated by similar % GFP+ T-cells and similar Mean Fluorescence Intensity (MFI) of the transfected T-cells (see FIG. 11A, FIG. 11B (% GFP+) and FIG. 11C and FIG. 11D (GFP MFI). This suggests that the antibody driven uptake was equally effective with both ionizable lipids and similar levels of ionizable lipid driven endosomal escape efficiencies were achieved. Lipid 7 and DLin-MC3-DMA lipids resulted in similar and lowest levels of GFP protein expression. Lipid 6 LNPs exhibited an intermediate level of GFP protein expression. The protein expression levels suggest that 9 carbon N-acyl substituent (lipid 3) enhances performance over an 11 carbon N-acyl substituents (of Lipids 6 and 7). The performance levels and relative order of LNP efficiency was preserved in all lipid nanoparticle formulation tested after 1 Freeze Thaw cycle (Post −80° C. storage) as illustrated by comparison of % GFP+ cells and GFP MFI values before and after −80° C. storage (FIG. 11A verses FIG. 11B and FIG. 11C versus FIG. 11D). While all lipid formulation tested were well tolerated by primary human T-cells (FIG. 11E); some dose dependent loss in T-cell viability was observed with lipids 7 and DLin-MC3-DMA.

### Example 24. In Vitro Protein Expression (GFP) in Primary Human T-Cells of Lipids 3, 4 and Comparator Lipid aCD3 Targeted LNPs (DLin-KC2-DMA and SM-102) Stored at 4° C. and after 1 Freeze-Thaw Cycle (Post −80° C. Storage)

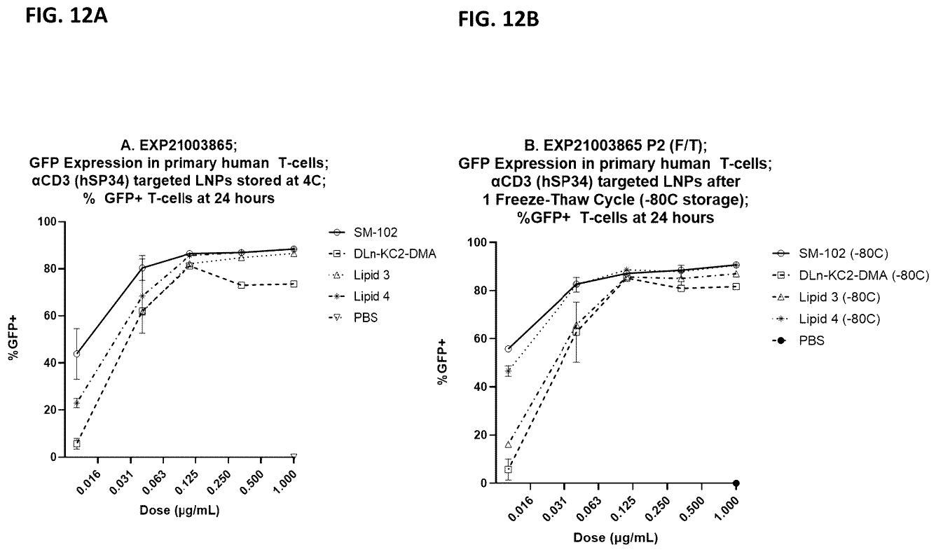

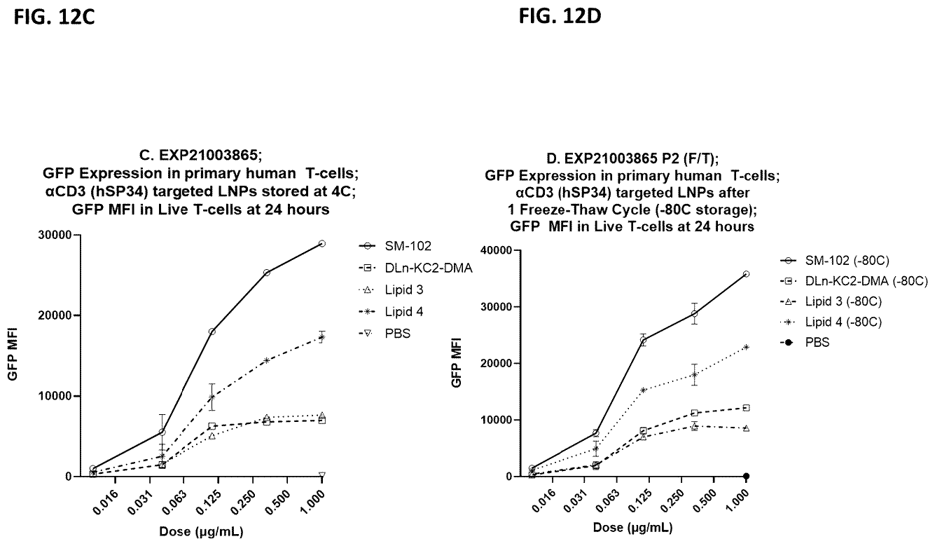

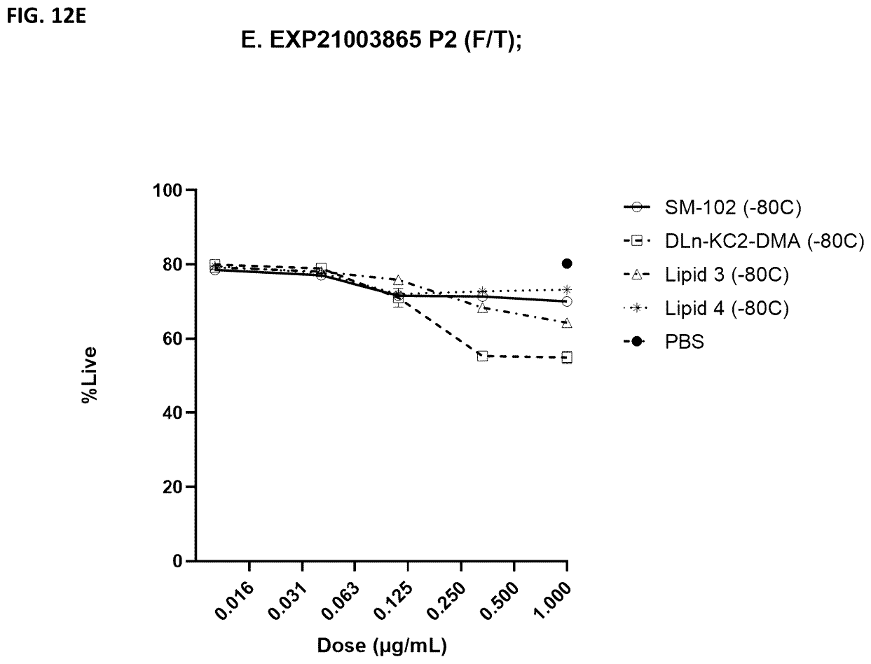

This example compares the GFP protein expression resulting from LNP's derived from Lipids 3 and 4 to LNPs made using comparator lipids DLin-KC2-DMA and SM-102. Nanoparticles bearing GFP encoding mRNA (and optionally a fluorescent dye label (DiI-C18-5DS)) were produced using the microfluidic mixing and buffer exchange processes described in Example 2. An αCD3 Fab-conjugate was incorporated into the parent LNPs to obtain the final Antibody targeted LNP formulation using the process described in Example 5. Particles thus produced were tested in vitro in primary human CD3+ T cells to assess reported gene expression. At ≥0.1 ug/mL dose, similar number of T-cells were transfected with SM-102 and Lipid 4 LNPs, however, at doses below 0.1 ug/mL, a larger fraction of T-cells were GFP+ with counted with SM-102 LNPs and the GFP MFI values were consistently about 2-fold higher at all doses suggesting more efficient cytosolic access/cytosolic release of encapsulated GFP mRNA relative to Lipid 4 LNPs. Lipid 3 and DLin-KC2-DMA LNPs performed similarly in terms of the fraction of T-cells accessed as well as copies of GFP protein expressed on a per cell basis and were at levels by 2-fold lower relative to Lipid 4 LNPs. This trend indicates that the oleoyl tail groups of Lipid 4 resulted in more efficient cytosolic access/cytosolic release of encapsulated GFP mRNA relative to the linoleoyl tail groups of Lipid 3. The performance levels and relative order of LNP efficiency was well preserved in all lipid nanoparticle formulation tested after 1 Freeze Thaw cycle (Post −80° C. storage) as illustrated by comparison of % GFP+ cells and GFP MFI values before and after −80° C. storage (FIG. 12A verses FIG. 12B and FIG. 12C versus FIG. 12D). Lipids 3, 4, and SM-102 LNPs were well tolerated by primary human T-cells (FIG. 12E); however, a slightly greater loss in T-cell viability was observed with DLin-KC2-DMA LNPs at the higher doses of 0.3 and 1 ug/mL.

### Example 25. In Vitro Protein Expression (GFP) in Primary Human T-Cells of Lipids 1, 3, 8 and Comparator Lipid aCD3 Targeted LNPs (DLin-KC2-DMA) Stored at 4° C. and after 1 Freeze-Thaw Cycle (Post −80° C. Storage)

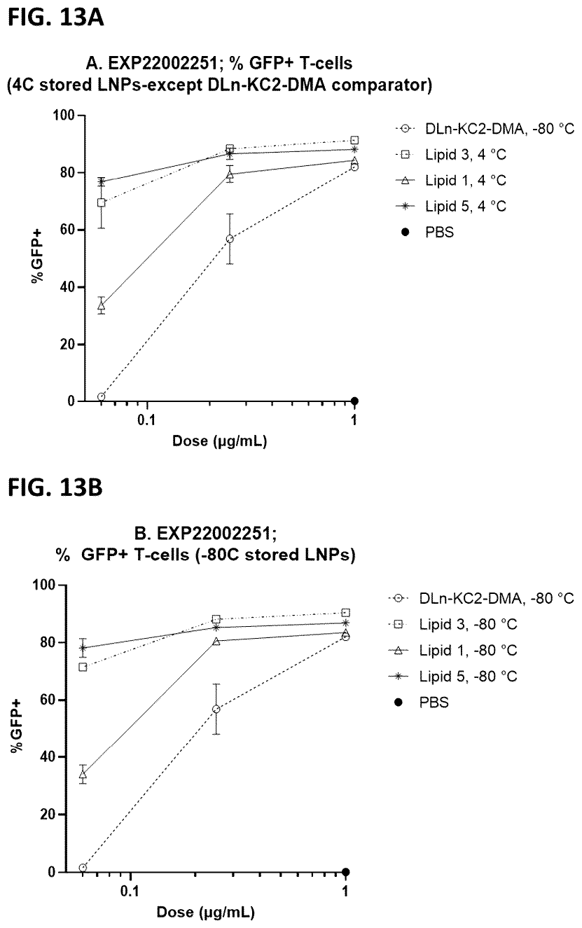

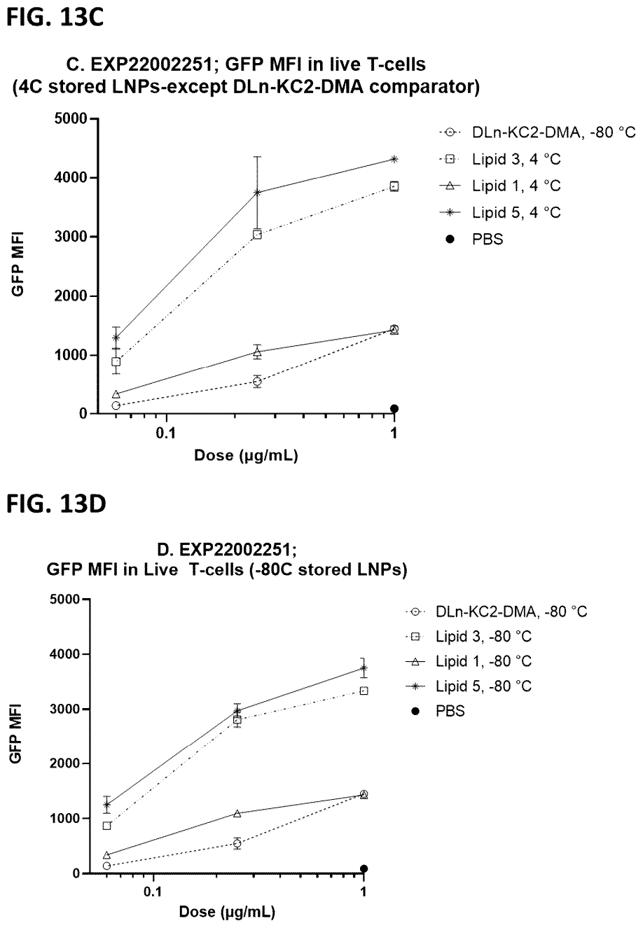

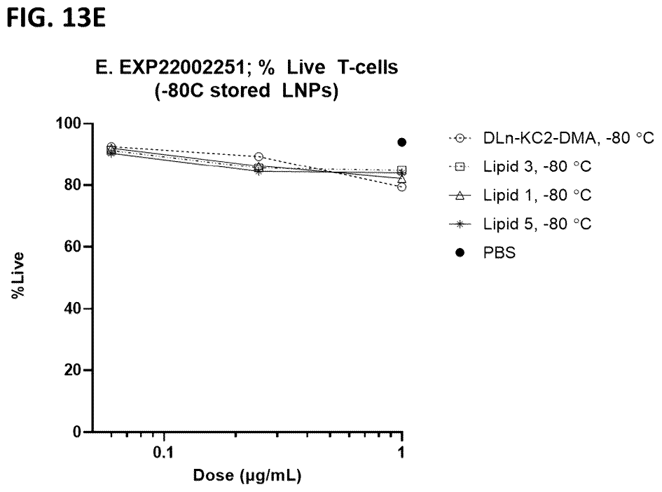

This example compares the GFP protein expression resulting from LNP's derived from Lipids 1, 3 and 5 to LNPs made using comparator lipid DLin-KC2-DMA. Nanoparticles bearing GFP encoding mRNA (and optionally a fluorescent dye label (DiI-C18-5DS)) were produced using the microfluidic mixing and buffer exchange processes described in Example 2. An αCD3 Fab-conjugate was incorporated into the parent LNPs to obtain the final Antibody targeted LNP formulation using the process described in Example 5. Particles thus produced were tested in vitro in primary human CD3+ T cells to assess reported gene expression. At all dose levels, Lipid 1 and DLin-KC2-DMA LNPs performed similarly both in terms of fraction of GFP+ T-cells (reflected by % GFP+ cells) and the copies of GFP protein produced on a per cell basis (reflected by GFP MFI values). Lipid 3 LNPs resulted in a roughly 3-fold higher fraction of GFP+ T-cells relative to Lipid 1 LNPs and the GFP MFI values were consistently about 3-fold higher at all doses suggesting more efficient cytosolic access/cytosolic release of encapsulated GFP mRNA with Lipid 3 LNPs relative to Lipid 1 indicating that the 9 carbon N-acyl substituent (of Lipid 3) is favorable for efficient cytosolic access/cytosolic release of encapsulated GFP mRNA relative to the 10 carbon N-acyl substituent (of Lipid 1). Highest GFP protein expression was observed with Lipid 5 LNPs as indicated by the higher % GFP+ and GFP MFI values at all dose levels. This indicates that further reduction in carbon number of the N-acyl substituent to 8 carbon skeleton further improves the mRNA delivery efficiency. However, as noted in Example 9, Lipid 5 LNPs exhibited larger size shifts in the buffer exchange and antibody conjugate insertion steps resulting in final LNP hydrodynamic diameters >150 nm indicating that 9-carbon N-acyl substitution skeleton proves optimal for LNP size (for enabling 0.2 micron sterile filtration) as well as the ability to induce reporter gene expression. The performance levels and relative order of LNP efficiency was well preserved in all formulation tested after 1 Freeze Thaw cycle (Post −80° C. storage) as illustrated by comparison of % GFP+ cells and GFP MFI values before and after −80° C. storage (FIG. 13A verses FIG. 13B and FIG. 13C versus FIG. 13D). Lipids 1, 3, 5 LNPs were well tolerated by primary human T-cells (FIG. 13E).

### Example 26. In Vitro Protein Expression (GFP) in Primary Human T-Cells of Lipids 1, 8 and Comparator Lipid aCD3 Targeted LNPs (DLin-KC2-DMA) Stored at 4° C. and after 1 Freeze-Thaw Cycle (Post −80° C. Storage)

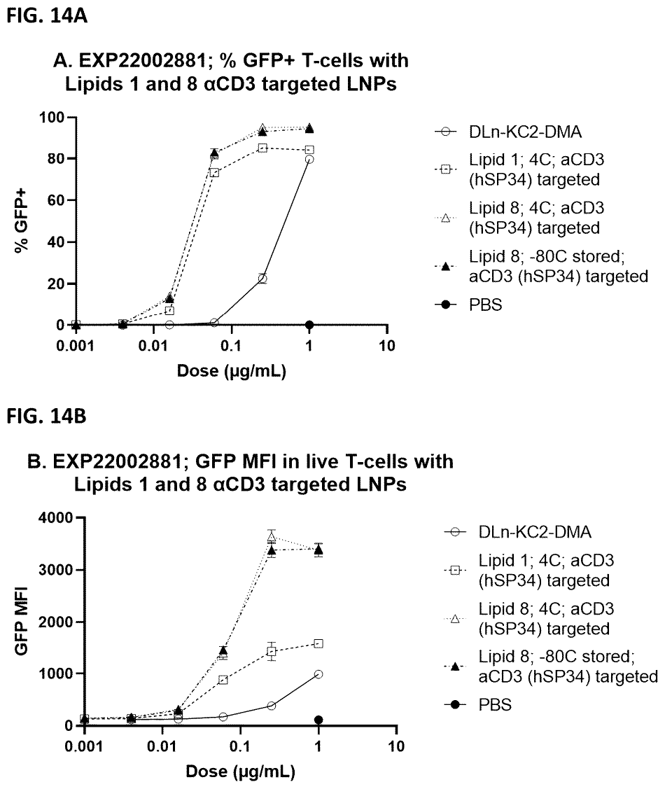

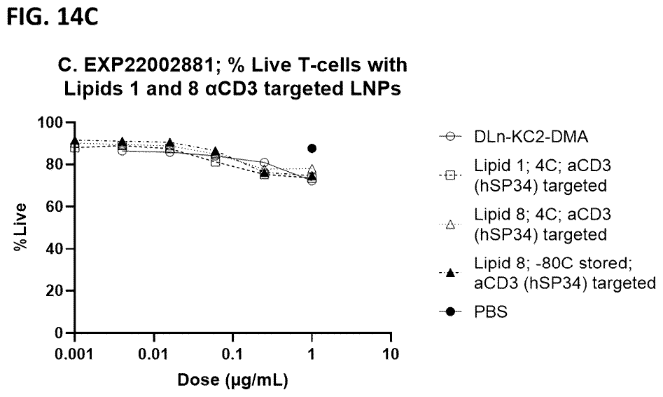

This example compares the GFP protein expression resulting from LNP's derived from Lipids 1 and 8. Nanoparticles bearing GFP encoding mRNA (and optionally a fluorescent dye label (DiI-C18-5DS)) were produced using the microfluidic mixing and buffer exchange processes described in Example 2. An αCD3 Fab-conjugate was incorporated into the parent LNPs to obtain the final Antibody targeted LNP formulation using the process described in Example 5. Particles thus produced were tested in vitro in primary human CD3+ T cells to assess reported gene expression. At all dose levels, both Lipid 1 and LNPs resulted in similar fraction of GFP+ T-cells (reflected by % GFP+ cells), however, Lipid 8 LNPs out-performed Lipid 1 LNPs by a factor of 3 in terms of copies of GFP protein produced on a per cell basis (reflected by GFP MFI values). This indicates that the β-ethyl octanoyl N-acyl substituent (of Lipid 8) improves cytosolic access/cytosolic release of encapsulated GFP mRNA relative to the α-ethyl octanoyl N-acyl substituent (of Lipid 1). The overall effect of the β-ethyl substitution is better LNP size distribution properties (relative to Lipid 1 as illustrated in Example 9) and gain in mRNA delivery efficiency resulting in a 2-fold improvement in reporter gene expression levels despite the 10 carbon N-acyl substitution pattern. This indicates that both LNP properties and delivery efficiency are tunable via size (carbon number) and geometry (Q-ethyl octanoyl versus α-ethyl octanoyl) of the N-acyl substituent. The performance of Lipid 8 LNP's was well preserved after 1 Freeze Thaw cycle (Post −80° C. storage) as illustrated by comparison of % GFP+ cells and GFP MFI values before and after −80° C. storage (FIG. 14A and FIG. 14B). Both Lipids 1 and 8 LNPs were well tolerated by primary human T-cells (FIG. 14C).

### Example 27. In Vitro Protein Expression (GFP) in Primary Human T-Cells of Lipids 8, 9, 10 and Comparator Lipid aCD3 Targeted LNPs (DLin-KC3-DMA) Stored at 4° C. and after 1 Freeze-Thaw Cycle (Post −80° C. Storage)

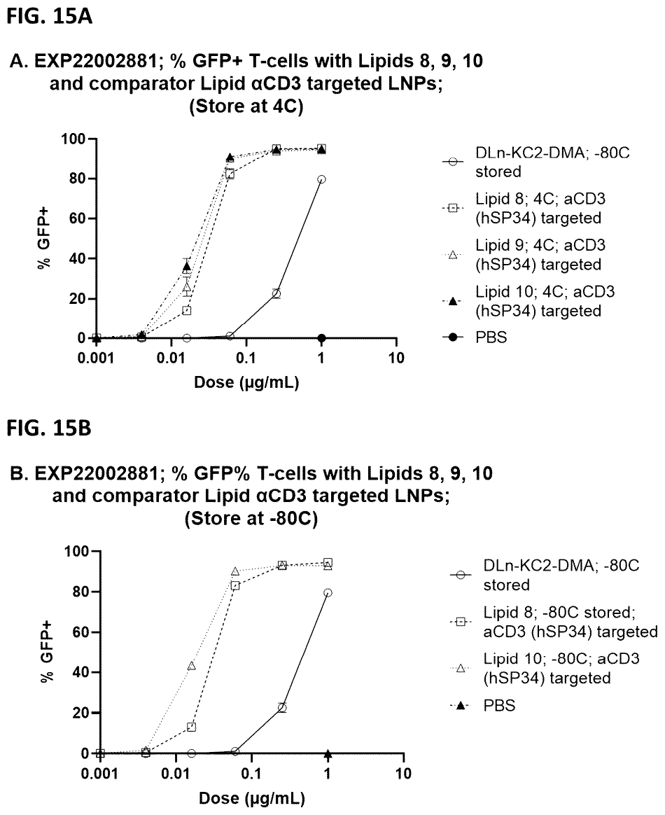

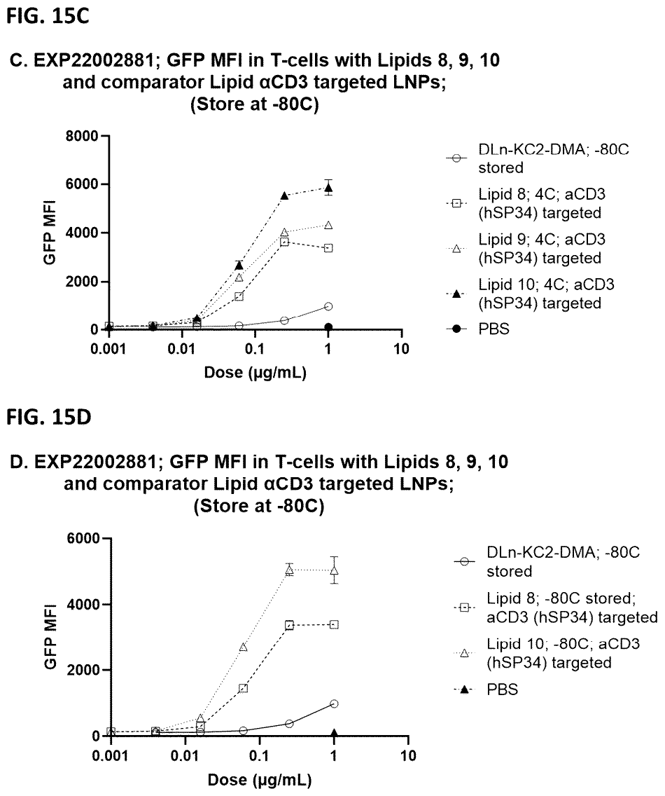

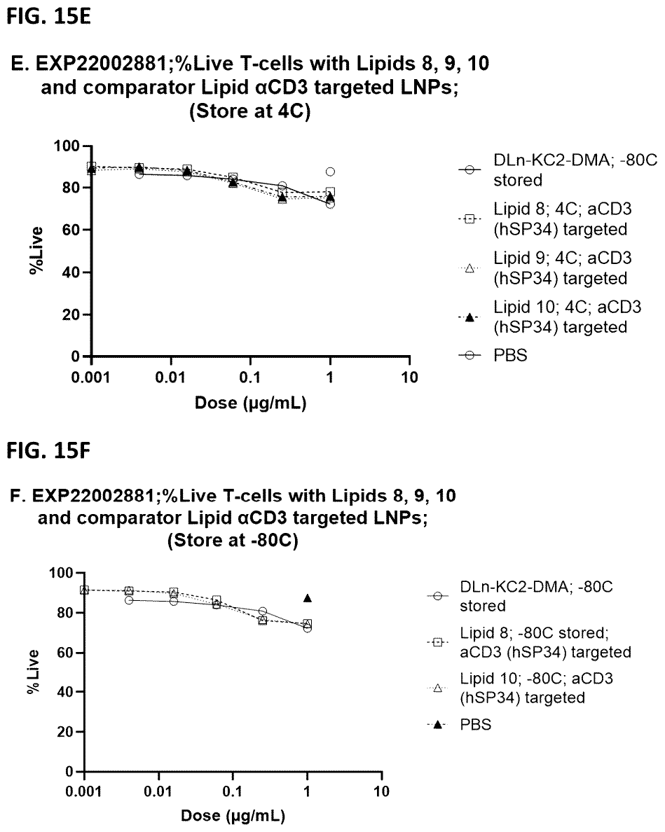

This example compares the GFP protein expression resulting from LNP's derived from Lipids 8, 9, 10 and comparator lipid DLin-KC2-DMA LNPs. Nanoparticles bearing GFP encoding mRNA (and optionally a fluorescent dye label (DiI-C18-5DS)) were produced using the microfluidic mixing and buffer exchange processes described in Example 2. An αCD3 Fab-conjugate was incorporated into the parent LNPs to obtain the final Antibody targeted LNP formulation using the process described in Example 5. Particles thus produced were tested in vitro in primary human CD3+ T cells to assess reported gene expression. As shown in FIGS. 15A and 15C, at all dose levels, Lipid 8 and 9 LNPs performed similarly both in terms of fraction of GFP+ T-cells (reflected by % GFP+ cells) and the copies of GFP protein produced on a per cell basis (reflected by GFP MFI values). Thus, the succinic acid derived 14 carbon N-acyl substituent (in Lipid 9) results in similar efficiency of cytosolic access/cytosolic release of the mRNA payload relative to the 10 carbon N-acyl substituent (of Lipid 8) indicating that introduction of a carboxylate ester (and added polarity of the 0-atoms) in N-acyl substituent balances and counters the loss of lipid efficiency seen with Lipids 6 and 7 (that both feature a 11 carbon N-acyl substituent). As illustrated in Example 10, Lipid 9 LNPs exhibit size distribution properties comparable to those of Lipids 6, 7 and 8 suggesting that biodegradable ester linkages (as in Lipid 9) can be introduced into the N-acyl substituent without loss of LNP size distribution properties. Lipid 10 LNPs out-performed Lipid 9 LNPs both in terms of fraction of GFP+ T-cells (reflected by % GFP+ cells) and the copies of GFP protein produced on a per cell basis (reflected by GFP MFI values) by a factor of 1.5. Suggesting that reduction in the total carbon count to 12 (in lipid 10) versus 14 (in Lipid 9) further improved lipid efficiency. Thus, analogous to trends observed with Lipids 3, 5, 6, 7 and 8 LNPs, the activity of succinic acid derived biodegradable N-acyl substituents in Lipids 9 and 10 is also tunable. The performance of Lipid 10 LNPs was well preserved after 1 Freeze Thaw cycle (Post −80° C. storage) as illustrated by comparison of % GFP+ cells and GFP MFI values before and after −80° C. storage (FIG. 15A verses FIG. 15B and FIG. 15C versus FIG. 15D). Lipids 9 and 10 LNPs were well tolerated by primary human T-cells (FIG. 15E and FIG. 15F).

### Example 28. In Vitro Protein Expression (GFP) in Primary Human T-Cells of Lipids 3, 4, 9, 15 and Comparator Lipid (DLin-KC2-DMA) aCD3 Targeted LNPs Stored at 4° C. and after 1 Freeze-Thaw Cycle (Post −80° C. Storage)

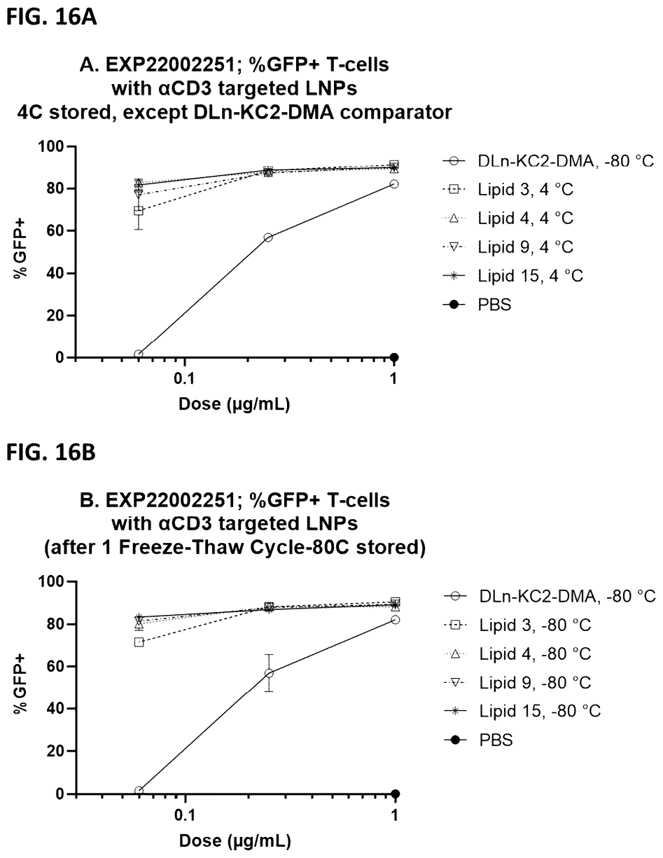

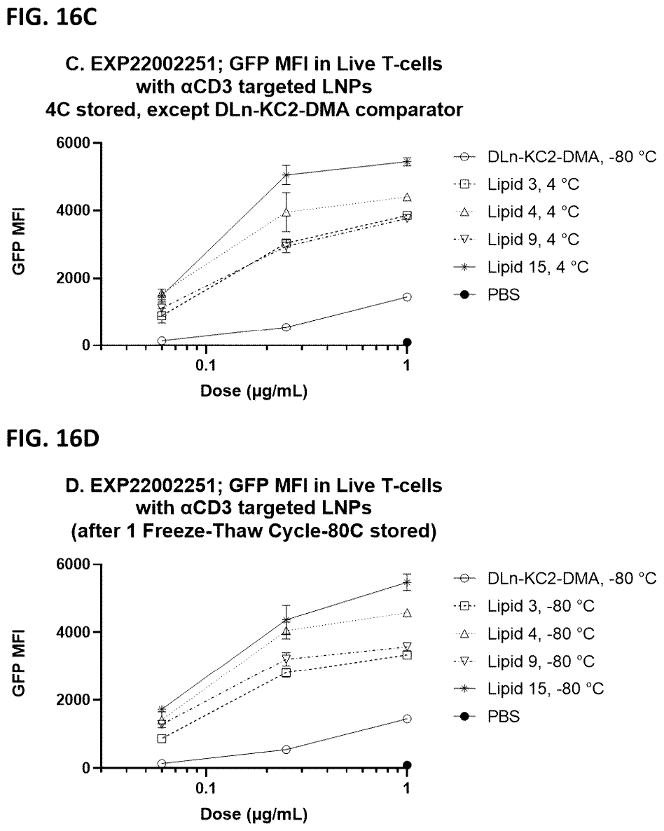

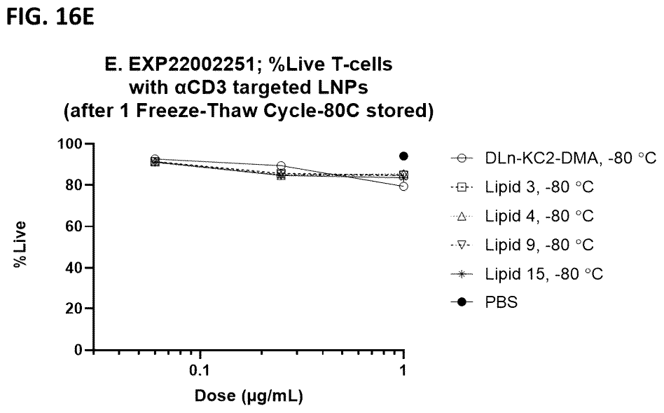

This example compares the GFP protein expression resulting from LNP's derived from Lipids 3, 4, 9, 15 and LNPs made using comparator lipid DLin-KC2-DMA. Nanoparticles bearing GFP encoding mRNA (and optionally a fluorescent dye label (DiI-C18-5DS)) were produced using the microfluidic mixing and buffer exchange processes described in Example 2. An αCD3 Fab-conjugate was incorporated into the parent LNPs to obtain the final Antibody targeted LNP formulation using the process described in Example 5. Particles thus produced were tested in vitro in primary human CD3+ T cells to assess reported gene expression. At all dose levels, performance improvements (both in terms of fraction of GFP+ T-cells as reflected by % GFP+ cells and the copies of GFP protein produced on a per cell basis as reflected by GFP MFI values) were observed between lipid 3 versus lipid 4 and between lipid 9 versus lipid 15 indicating that the oleoyl tail groups (in Lipids 4 and 15) result in improved performance over the corresponding linoleoyl tail groups (in Lipids 3 and 9). Thus, gains in lipid oxidative stability via lower lipid tail unsaturation (with singly unsaturated oleic acid) without loss of LNP efficiency typically observed with the lower lipid membrane fluidity. Furthermore, in contrast to Lipid 4 where poorer LNP size distribution properties were observed (relative to Lipid 3), Lipid 15 LNPs exhibited size distribution properties similar to those observed with Lipid 9 LNPs indicating that the N-acyl substituent plays a more significant role in determining the LNPs size distribution properties compared to the role of the lipid tail groups. The performance levels and relative order of LNP efficiency was well preserved in all formulation tested after 1 Freeze Thaw cycle (Post −80° C. storage) as illustrated by comparison of % GFP+ cells and GFP MFI values before and after −80° C. storage (FIG. 16 A verses FIG. 16B and FIG. 16C versus FIG. 16D). Lipids 3, 4, 9, and 15 LNPs were well tolerated by primary human T-cells (FIG. 16E).

### Example 29. In Vitro Protein Expression (GFP) in Primary Human T-Cells of Lipids 3, 4, 9, 15 aCD8 (TRX-2) Targeted LNPs and the Corresponding Non-Targeted Parent LNPs (Both Stored at 4° C.)

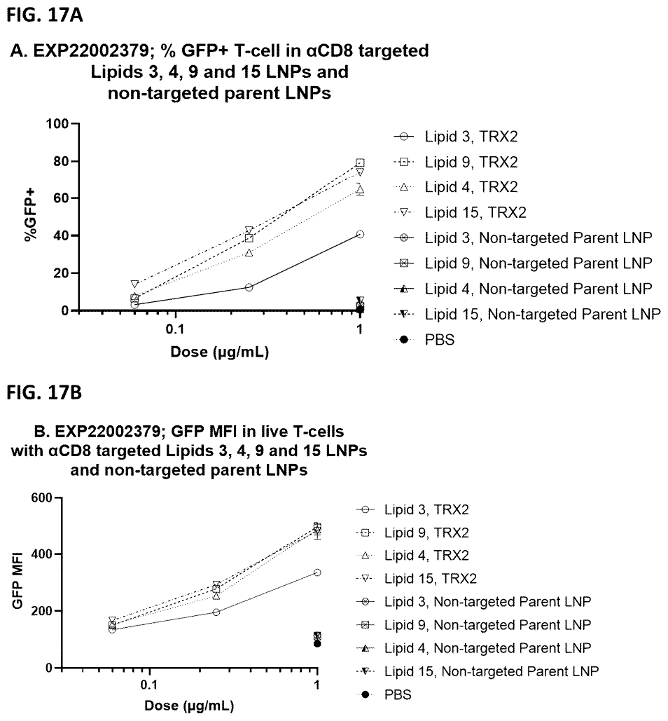

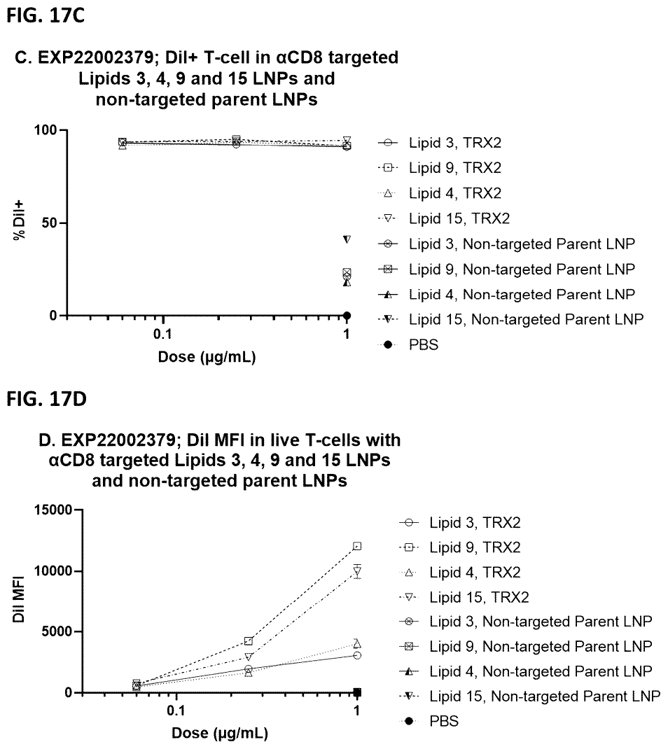

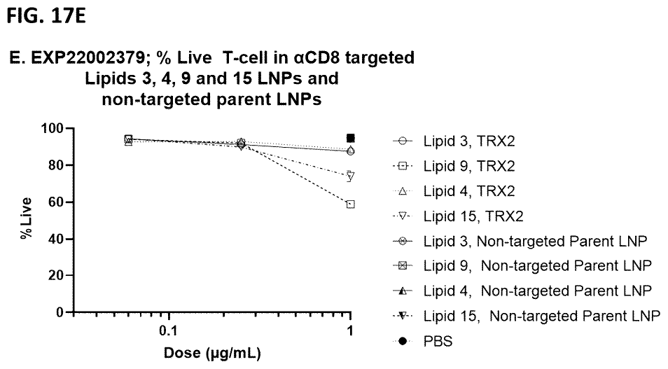

This example compares the GFP protein expression resulting from LNP's derived from Lipids 3, 4, 9, 15 and the corresponding non-targeted parent LNPs. Nanoparticles bearing GFP encoding mRNA (and optionally a fluorescent dye label (DiI-C18-5DS)) were produced using the microfluidic mixing and buffer exchange processes described in Example 2. An αCD8 Fab-conjugate, TRX2, were incorporated into the parent LNPs to obtain the final Antibody targeted LNP formulation using the process described in Example 5. Particles thus produced were tested in vitro in primary human CD8 T cells to assess reported gene expression. Additionally, parent LNPs (without any targeting Fab conjugate incorporated into the LNP corona) were tested to check any non-specific uptake in the CD8 T-cell population. As seen in FIG. 17A and FIG. 17B, Lipids 9 and 15 resulted in similar levels of GFP protein expression the CD8 T-cell population (both in terms of fraction of GFP+ T-cells as reflected by % GFP+ cells and the copies of GFP protein produced on a per cell basis as reflected by GFP MFI values). FIG. 17C and FIG. 17D, show the % DiI+(dye) T-cells and the DiI MFI reflective of the relative levels of DiI dye labeled LNPs taken up by the CD8 T-cell population. Notably, similar % DiI+ T-cells were observed in all targeted formulations while buffer control-like dye levels (DiI MFI values, FIG. 17 D) were seen in parent (non-targeted) formulations confirming the role of the TRX2 targeting Fab in association and uptake into CD8 T-cells. As expected, no GFP protein expression was observed in the non-targeted parent LNP formulations indicating lipid chemistry did not play a role this TRX2 mediated cellular uptake mechanism. Lipids 3, 4, 9, and 15 αCD8 (TRX2) targeted LNPs were well tolerated by primary human T-cells (FIG. 17E) with cell viability trending a measurable lower in Lipid 9 and Lipid 15 formulations possibly due to the higher levels of GFP protein expression observed with these lipids (as indicated by the higher GFP MFI values seen FIG. 17B).

### Example 30. In Vitro Protein Expression (GFP) in Primary Human T-Cells of Lipids 3, 4, 9, 15 aCD8 (T8) Targeted LNPs and the Corresponding Non-Targeted Parent LNPs (Both Stored at 4° C.)

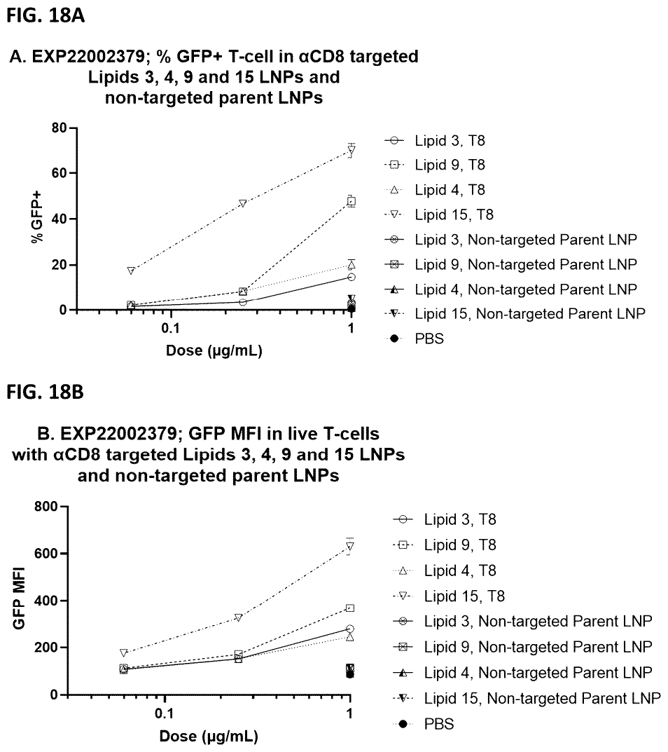

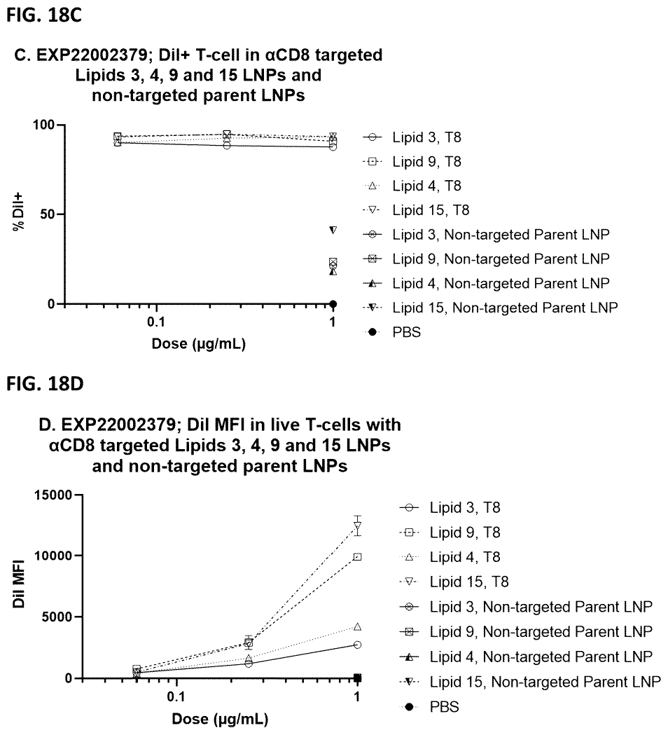

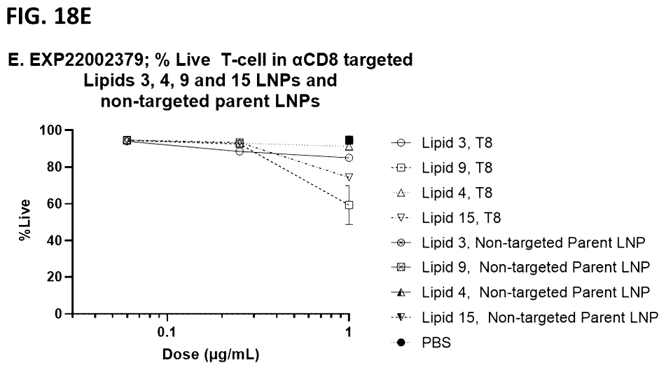

This example compares the GFP protein expression resulting from LNP's derived from Lipids 3, 4, 9, 15 and the corresponding non-targeted parent LNPs (both after one Freeze-Thaw cycle). Nanoparticles bearing GFP encoding mRNA (and optionally a fluorescent dye label (DiI-C18-5DS)) were produced using the microfluidic mixing and buffer exchange processes described in Example 2. An αCD8 Fab-conjugate, T8, was incorporated into the parent LNPs to obtain the final Antibody targeted LNP formulation using the process described in Example 5. Particles thus produced were tested in vitro in primary human CD8 T cells to assess reported gene expression. Additionally, parent LNPs (without any targeting Fab conjugate incorporated into the LNP corona) were tested to check any non-specific uptake in the CD8 T-cell population. As seen in FIG. 18A and FIG. 18B, with this T8 αCD8 targeting strategy, Lipid 15 LNPs out-performed Lipid 9 LNPs with 2-3 fold higher reporter gene expression observed (both in terms of fraction of GFP+ T-cells as reflected by % GFP+ cells and the copies of GFP protein produced on a per cell basis as reflected by GFP MFI values). FIG. 18C and FIG. 18D, show the % DiI+(dye) T-cells and the DiI MFI reflective of the relative levels DiI dye labelled LNPs taken up by the CD8 T-cell population. Notably, similar % DiI+ T-cells were observed in all targeted formulations while buffer control-like dye levels (DiI MFI values, FIG. 18 D) were seen in parent (non-targeted) formulations confirming the role of the T8 targeting Fab in association and uptake into CD8 T-cells. As expected, no GFP protein expression was observed with the non-targeted parent LNP formulations indicating lipid chemistry did not play a role this T8 mediated cellular uptake mechanism. Lipids 3, 4, 9, and 15 αCD8 (T8) targeted LNPs were well tolerated by primary human T-cells (FIG. 18E) with cell viability trending a measurable lower in Lipid 9 and Lipid 15 formulations possibly due to the higher levels of GFP protein expression observed with these lipids (as indicated by the higher GFP MFI values seen FIG. 18B).

### Example 31. In Vitro Protein Expression (GFP) in Primary Human T-Cells of Lipids 2, 3, 31 and 32 and Comparator Lipid (DLin-KC2-DMA) aCD3 (Hsp34) Targeted LNPs Stored at 4° C.

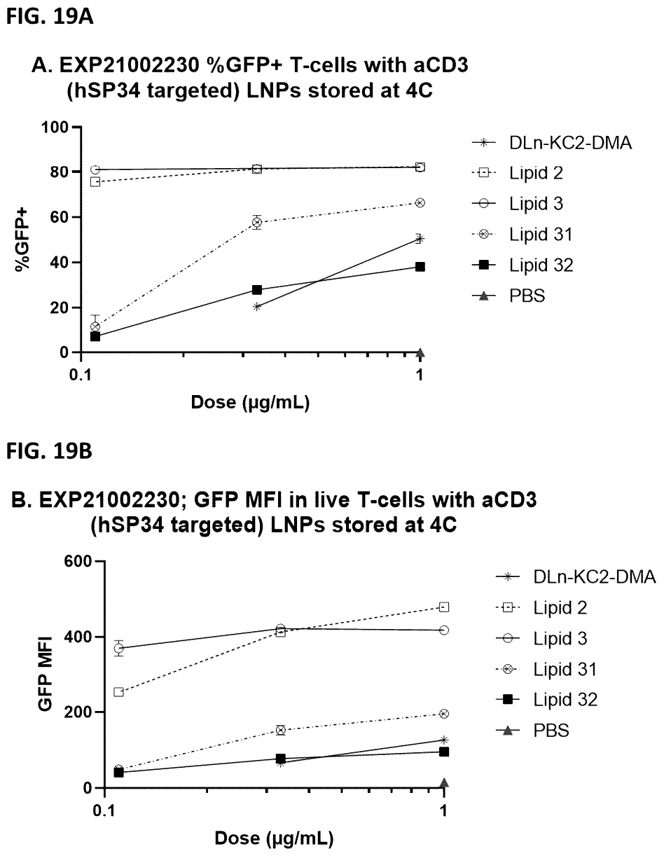

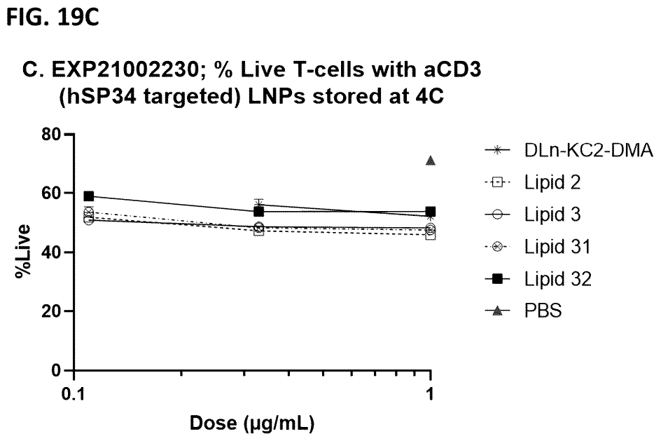

This example compares the GFP protein expression resulting from LNP's derived from Lipids 2, 3, 31 and 32 to LNPs made using comparator lipid DLin-KC2-DMA. Nanoparticles bearing GFP encoding mRNA (and optionally a fluorescent dye label (DiI-C18-5DS)) were produced using the microfluidic mixing and buffer exchange processes described in Example 2. An αCD3 Fab-conjugate was incorporated into the parent LNPs to obtain the final Antibody targeted LNP formulation using the process described in Example 5. Particles thus produced were tested in vitro in primary human CD3+ T cells to assess reported gene expression. At all dose levels, Lipid 2 and 3 out-performed Lipid 31, 32 and DLin-KC2-DMA LNPs both in terms of fraction of GFP+ T-cells (reflected by % GFP+ cells) and the copies of GFP protein produced on a per cell basis (reflected by GFP MFI values) (FIG. 19A and FIG. 19B). This is consistent with higher apparent pKa of Lipid 31 and Lipid 32 LNPs relative to Lipid 2 and 3 LNPs and less pronounced change in LNP charge state under acidic endosomal pH conditions (as described in Example 13) and consequently lower levels of cytosolic access expected with Lipids 31 and 32. Lipids 2, 3, 31 and 32 LNPs were well tolerated by primary human T-cells (FIG. 19C).

### Example 32. In Vitro Protein Expression (GFP) in Primary Human T-Cells of Lipids 3, 33 and 34 and Comparator Lipid (DLin-KC2-DMA) aCD3 Targeted LNPs Stored at 4° C. and after 1 Freeze-Thaw Cycle (Post −80° C. Storage) and Non-Binding (Mutated OKT8) Antibody Targeted LNPs

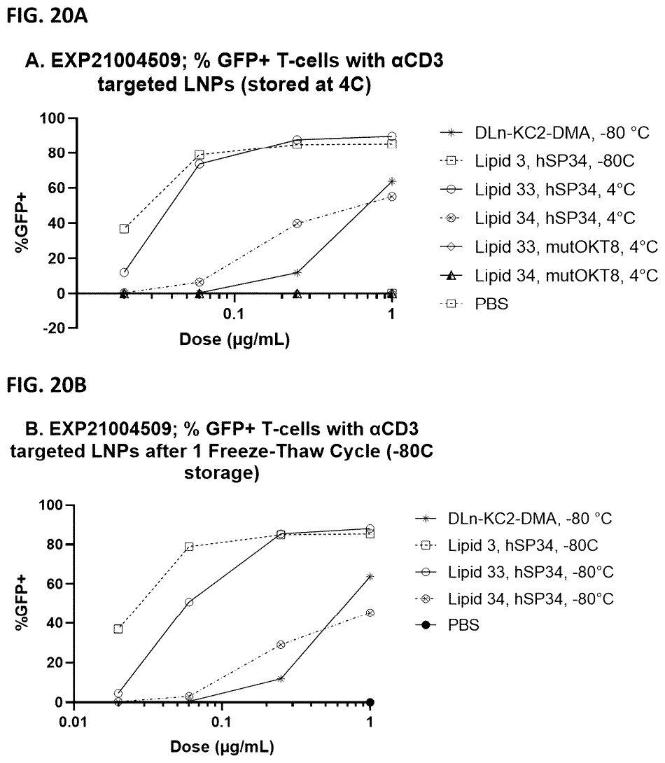

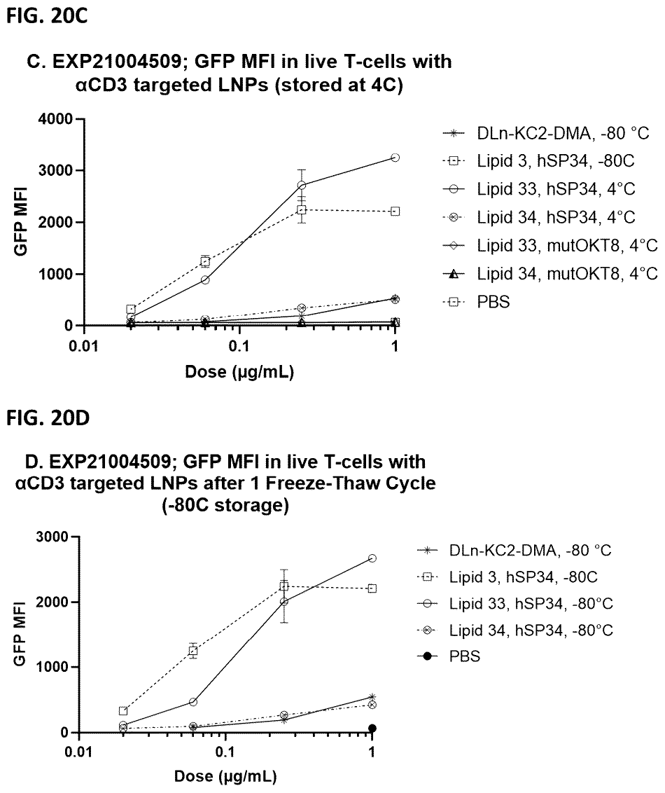

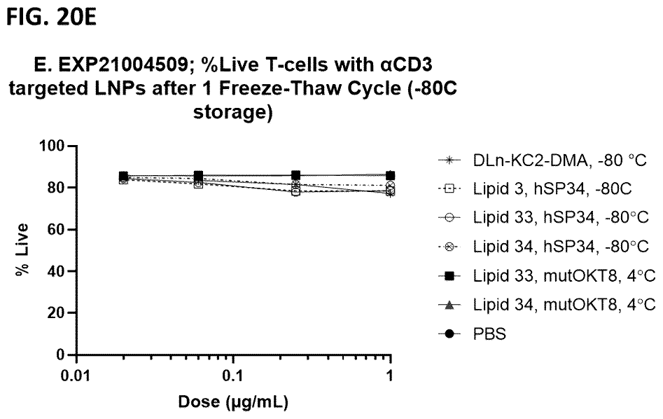

This example compares the GFP protein expression resulting from LNP's derived from Lipids 3, 31, 32 and LNPs made using comparator lipid DLin-KC2-DMA. Nanoparticles bearing GFP encoding mRNA (and optionally a fluorescent dye label (DiI-C18-5DS)) were produced using the microfluidic mixing and buffer exchange processes described in Example 2. An αCD3 Fab-conjugate was incorporated into the parent LNPs to obtain the final Antibody targeted LNP formulation using the process described in Example 5. Additionally, mock targeted LNPs were produced by incorporation of a non-binding mutated Antibody Fab (mut-OKT8) conjugate into the parent LNPs using the Antibody conjugate insertion method described in Example 4. Particles thus produced were tested in vitro in primary human CD3+ T cells to assess reported gene expression. At all dose levels, Lipid 3 and 33 out-performed Lipid 34 and DLin-KC2-DMA LNPs both in terms of fraction of GFP+ T-cells (reflected by % GFP+ cells) and the copies of GFP protein produced on a per cell basis (reflected by GFP MFI values). Furthermore, mut-OKT8 functional Lipid 33 and Lipid 34 LNPs did not result in any protein expression confirming the role of the αCD3 Antibody (hSP34) in the cellular uptake mechanism observed with these lipid formulations. The performance levels and relative order of LNP efficiency was well preserved in all formulations tested after 1 Freeze Thaw cycle (Post −80° C. storage) as illustrated by comparison of % GFP+ cells and GFP MFI values before and after −80° C. storage (FIG. 20A verses FIG. 20B and FIG. 20C versus FIG. 20D). Lipids 3, 33 and 34 LNPs were well tolerated by primary human T-cells (FIG. 20E).

### Example 33. In Vitro Car Protein Expression (M1 Tagged Extracellular Domain) in Primary Human T-Cells of Lipids 3, 4, 9 and 34, aCD3 Targeted aCD20 Car-RNA LNPs Stored at 4° C. and after 1 Freeze-Thaw Cycle (Post −80° C. Storage)

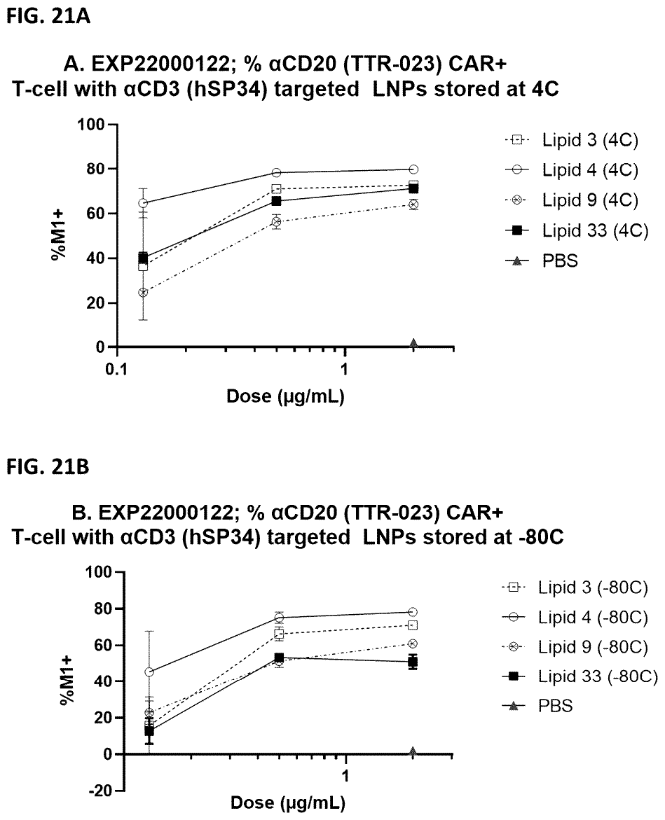

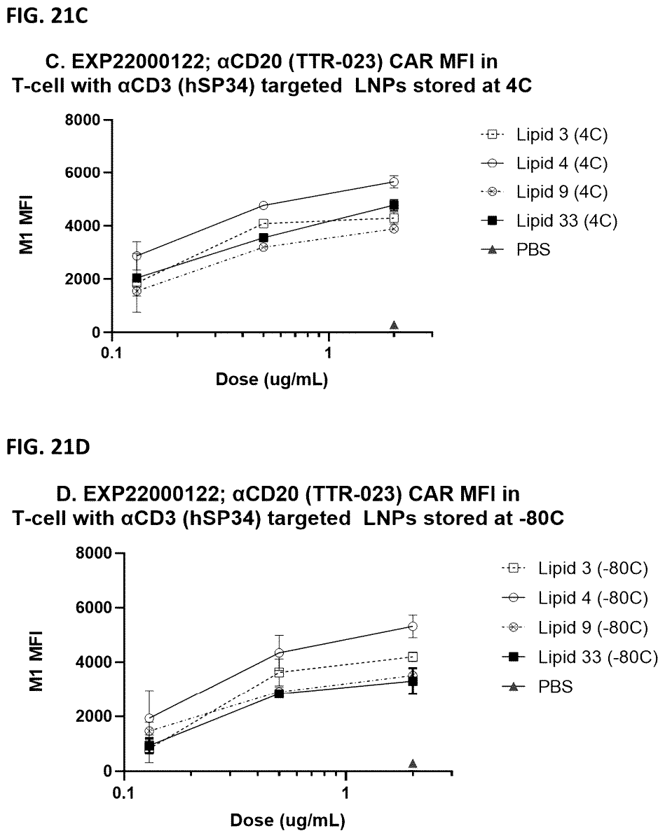

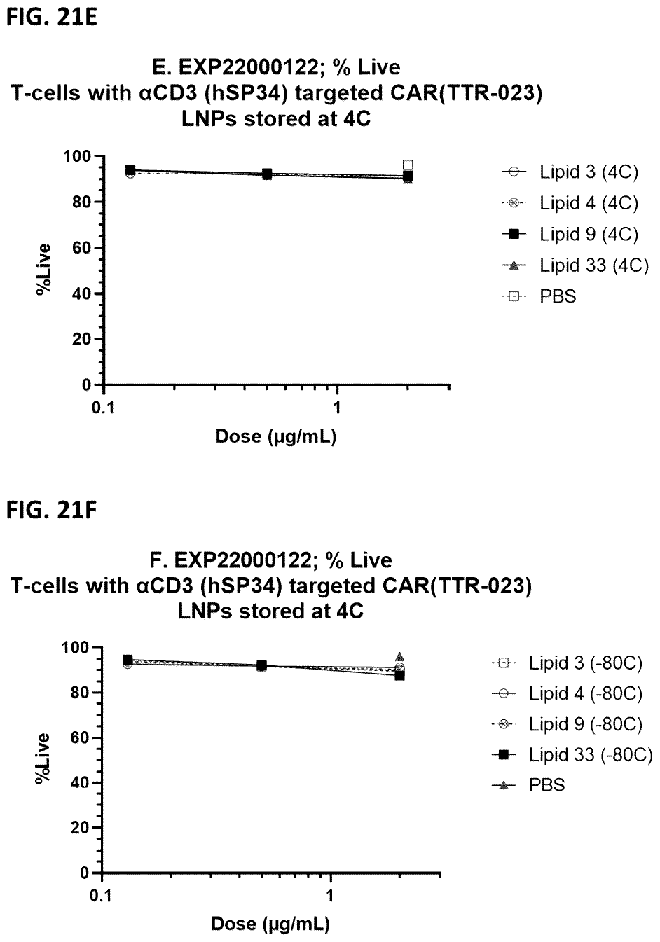

This example compares the αCD20 CAR (TTR-023) protein expression resulting from LNP's derived from Lipids 3, 4, 9 and 33. Nanoparticles bearing an αCD20 CAR encoding mRNA (TTR-023) were produced using the microfluidic mixing and buffer exchange processes described in Example 2. An αCD3 Fab-conjugate (hSP34) was incorporated into the parent LNPs to obtain the final Antibody targeted LNP formulation using the process described in Example 5. Particles thus produced were tested in vitro in primary human CD3+ T cells to assess CAR expression via detection of an M1 tag on the extracellular domain of the TTR-023 transmembrane CAR protein. At all dose levels tested, higher CAR levels were detected with lipids 3 and 4 relative to lipids 9 and 33 with Lipid 4 performing the best in this αCD3 targeting pathway. The relative levels of CAR expression are consistent with oleoyl tail group of Lipid 4 resulting in improved performance over the corresponding linoleoyl tail group of Lipids 3 and 9 seen with reported (GFP) gene expression in Examples 28, 29 and 30. Furthermore, this illustrates that this relative order of lipid efficiency is preserved between a reporter gene (GFP) and a therapeutic cargo (TTR-023 CAR protein) in this αCD3 mediated targeting and cellular uptake mechanism. The performance levels and relative order of LNP efficiency was well preserved in Lipid 3, 4 and 9 LNPs after 1 Freeze Thaw cycle (Post −80° C. storage). In contrast, a drop in CAR expression was detected at all doses of Lipid 33 LNP as illustrated by comparison of % M1+ cells and M1 MFI values before (4° C. stored) and after −80° C. storage (FIG. 21A verses FIG. 21B and FIG. 21C versus FIG. 21D). Lipids 3, 4, 9, and 33 LNPs were well tolerated by primary human T-cells (FIG. 21E and FIG. 21F).

### Example 34. In Vitro Car Protein Expression (M1 Tagged Extracellular Domain) in Primary Human T-Cells of Lipids 3, 4, 9 and 34, aCD8 (T8) Targeted aCD20 Car-RNA LNPs Stored at 4° C.

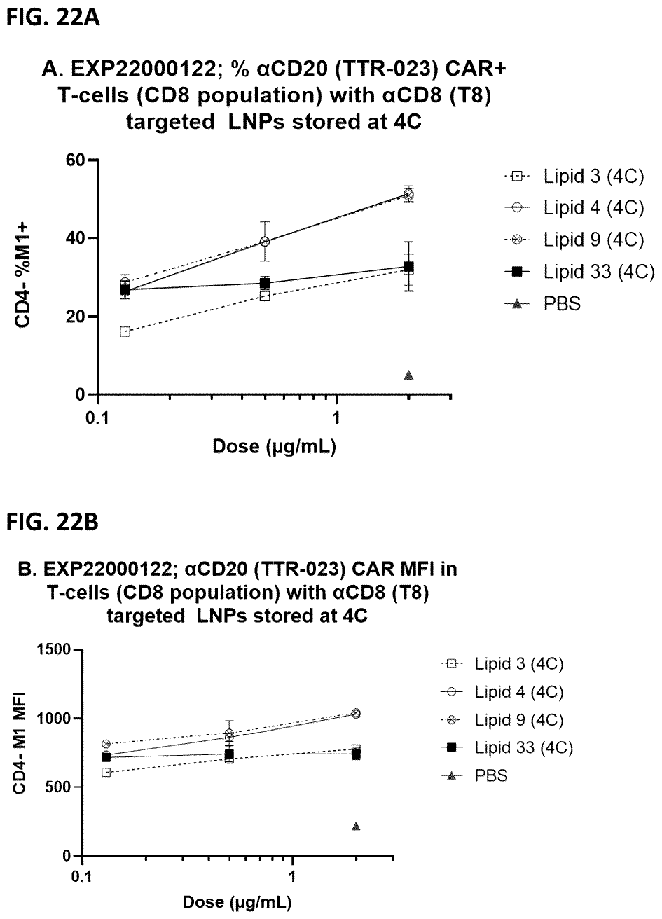

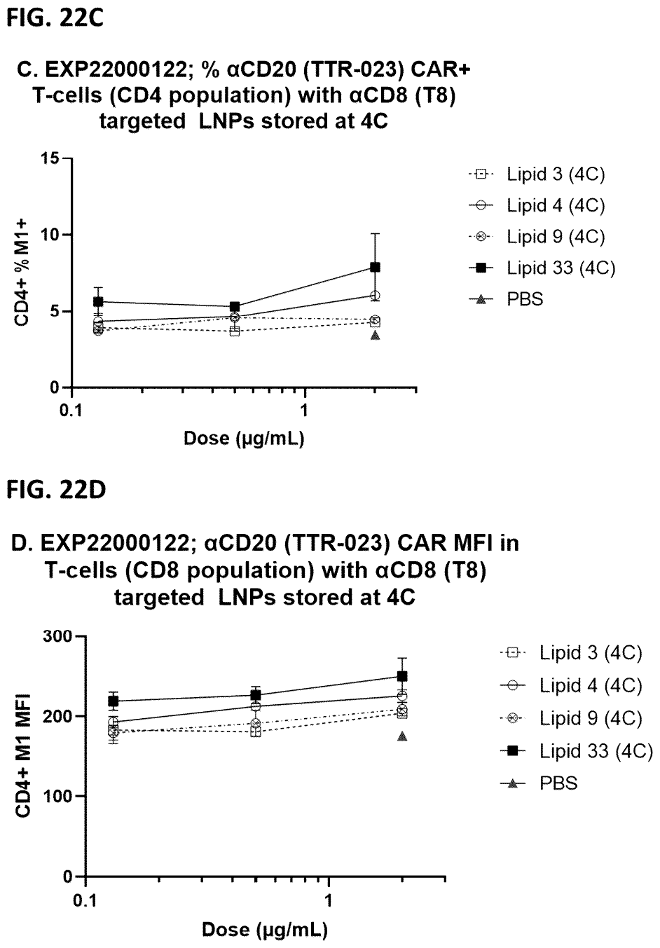

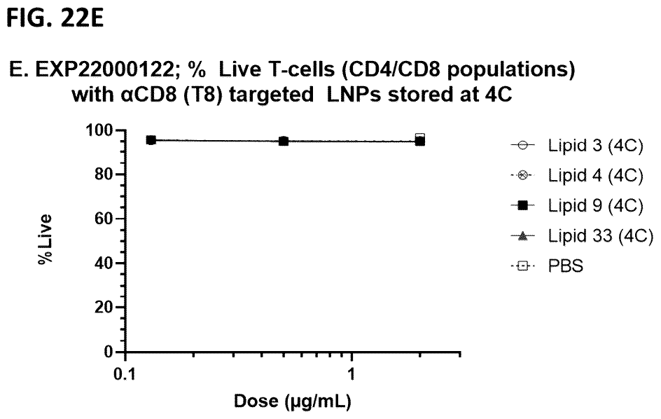

This example compares the αCD20 CAR (TTR-023) protein expression resulting from LNP's derived from Lipids 3, 4, 9 and 33. Nanoparticles bearing an αCD20 CAR encoding mRNA (TTR-023) were produced using the microfluidic mixing and buffer exchange processes described in Example 2. An αCD8 Fab-conjugate (T8) was incorporated into the parent LNPs to obtain the final Antibody targeted LNP formulation using the process described in Example 5. Particles thus produced were tested in vitro in primary human CD3+ T cells to assess CAR expression via detection of an M1 tag on the extracellular domain of the TTR-023 transmembrane CAR protein. Transfected cells were gated as CD4+(CD4 population) and CD4− (CD8 population) and CAR expression was monitored (as % M1+ and M1 MFI) in the two populations to assess specificity in the CD8 population with the αCD8 (T8) targeting strategy. At all dose levels tested, higher CAR levels in the CD4− population (CD8 cells, as illustrated by the M1% and M1 MFI values in FIG. 22A and FIG. 22B) were detected with lipids 4 and 9 relative to lipids 3 and 33 with Lipids 4 and 9 performing equally well in this CD8 targeting pathway. CAR levels similar to buffer control (PBS) were detected in the CD4+ population (CD4 cells, as illustrated by the M1% and M1 MFI values in FIG. 22C and FIG. 22D) confirming that a T8 antibody enables receptor mediated uptake specifically into CD8 T-cells. Lipids 3, 4, 9, and 33 LNPs were well tolerated by primary human T-cells (FIG. 22E).

### Example 35. In Vitro Car Protein Expression (M1 Tagged Extracellular Domain) in Primary Human T-Cells of Lipids 3, 4, 9 and 34, aCD8 (T8) Targeted aCD20 Car-RNA LNPs after 1 Freeze-Thaw Cycle (Post −80° C. Storage)

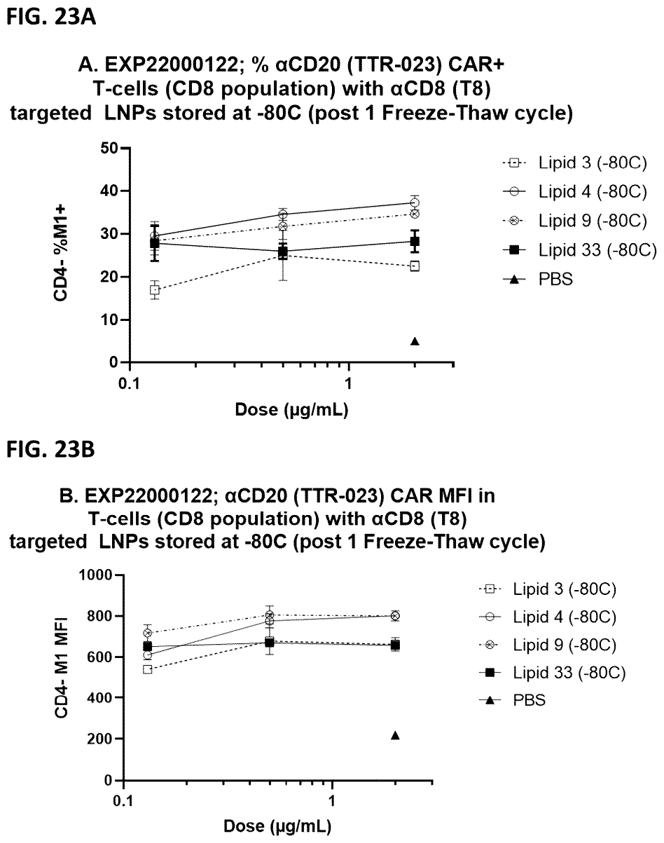

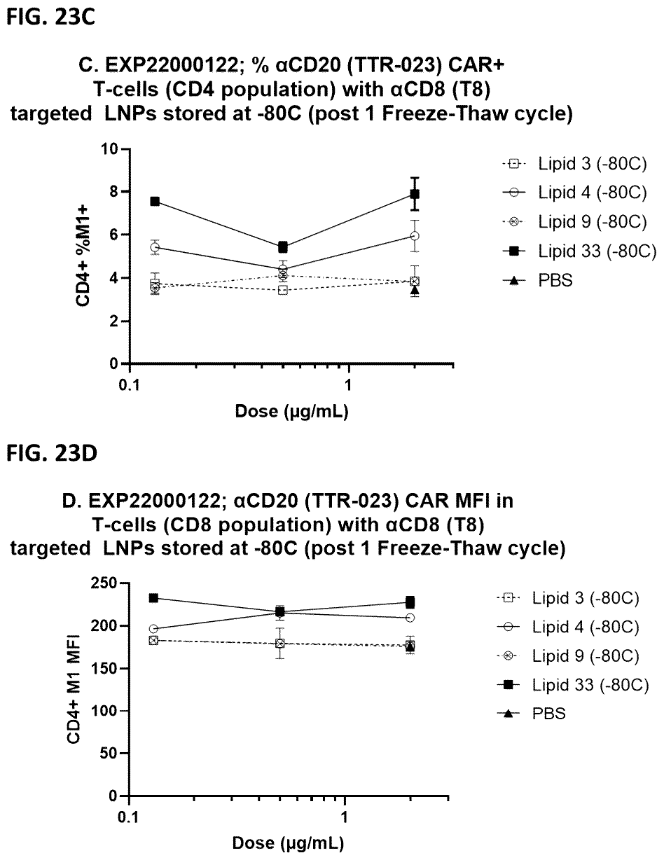

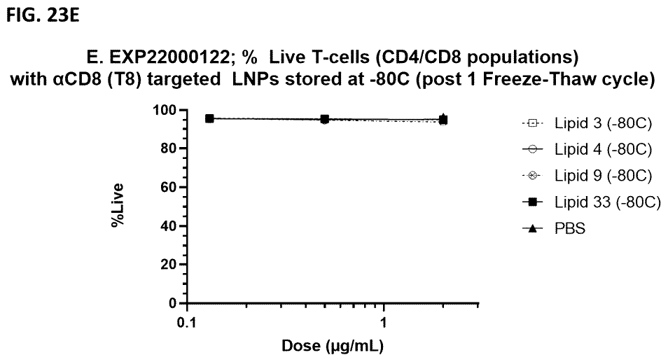

This example compares the αCD20 CAR (TTR-023) protein expression resulting from LNP's derived from Lipids 3, 4, 9 and 33 after 1 Freeze-Thaw cycle (post −80° C. storage). Nanoparticles bearing an αCD20 CAR encoding mRNA (TTR-023) were produced using the microfluidic mixing and buffer exchange processes described in Example 2. An αCD8 Fab-conjugate (T8) was incorporated into the parent LNPs to obtain the final Antibody targeted LNP formulation using the process described in Example 5. Particles thus produced were subjected to 1 Freeze-Thaw cycle and then tested in vitro in primary human CD3+ T cells to assess CAR expression via detection of an M1 tag on the extracellular domain of the TTR-023 transmembrane CAR protein. Transfected T-cells were gated as CD4+(CD4 population) and CD4− (CD8 population) and CAR expression was monitored (as % M1+ and M1 MFI) in the two populations to assess specificity in the CD8 population with the αCD8 (T8) targeting strategy. At all dose levels tested, higher CAR levels in the CD4− population (CD8 cells, as illustrated by the M1% and M1 MFI values in FIG. 23A and FIG. 23B) were detected with lipids 4 and 9 relative to lipids 3 and 33 while Lipids 4 and 9 performed equally well in this CD8 targeting pathway. CAR levels similar to buffer control (PBS) were detected in the CD4+ population (CD4 cells, as illustrated by the M1% and M1 MFI values in FIG. 23C and FIG. 23D) confirming that a T8 antibody enables receptor mediated uptake specifically into CD8 T-cells. Both levels of CAR expression and specificity to CD8 T-cells were observed with particles that had been subjected to 1 Freeze-Thaw cycle confirming that the integrity and function of the formulations were preserved. Lipids 3, 4, 9, and 33 LNPs were well tolerated by primary human T-cells (FIG. 23E).

### Example 36. GFP Protein Expression and LNP Association (Measured as DiI-Dye Fluorescence) in CD8 and CD4 T-Cell with Lipid 9, 15, DLin-KC3-DMA Lipid aCD3 (Hsp34) Targeted LNPs in Human Whole Blood

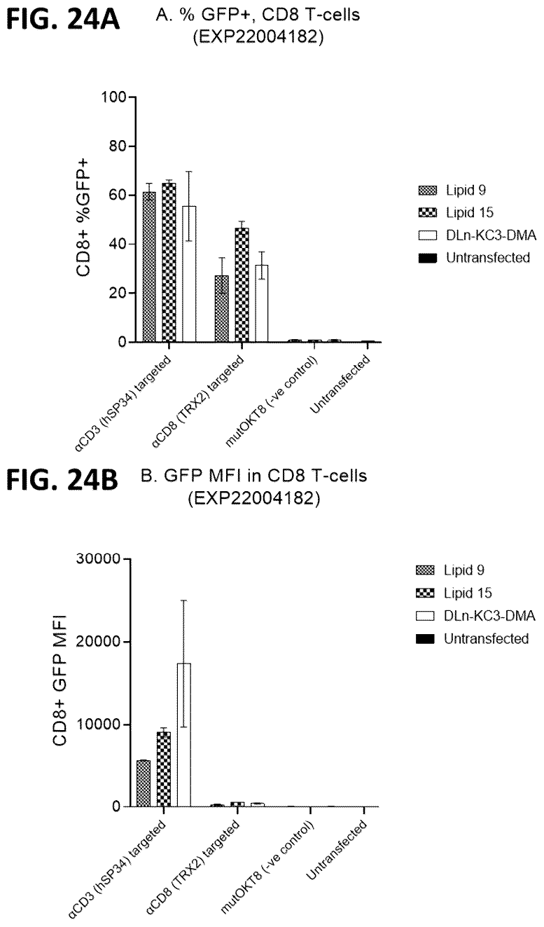

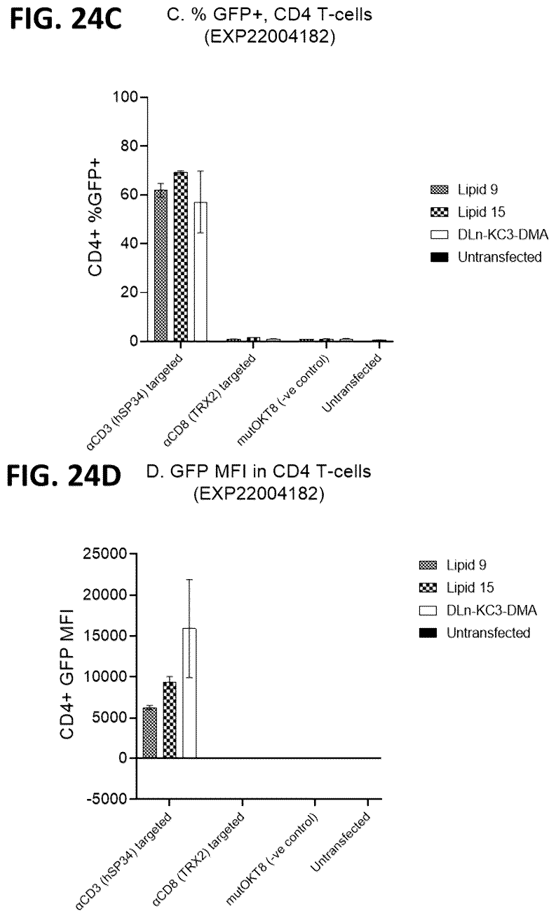

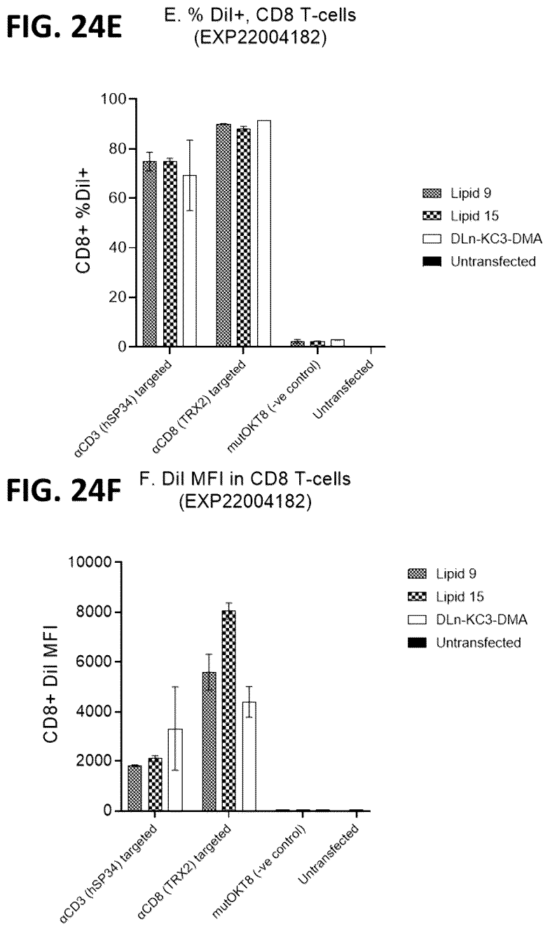

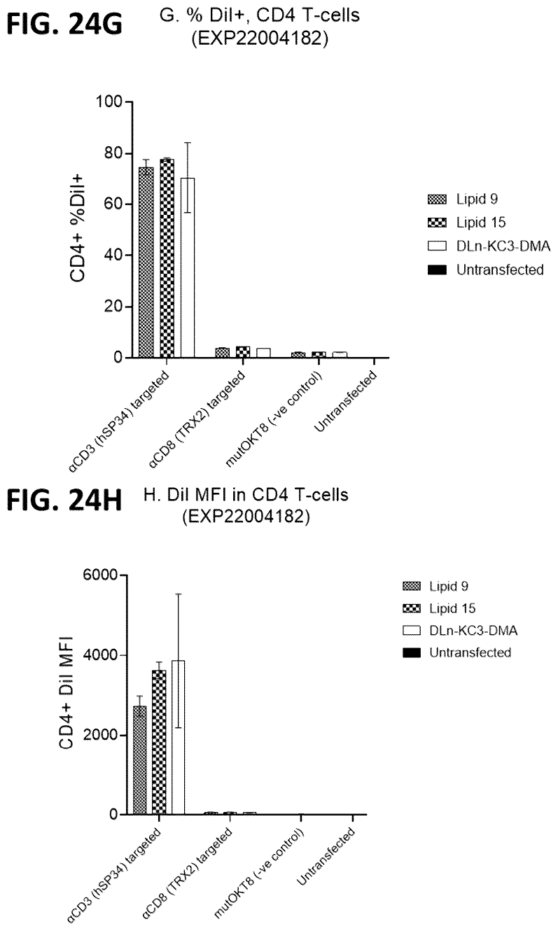

Lipid 9, 15 and DLin-KC3-DMAαCD3 and αCD8 targeted (and a non-binding Antibody, mutOKT8, as a negative control) GFP mRNA LNPs were dosed to human venous whole blood, incubated for 24 hours and analyzed using the protocol described in Example 21 for whole blood transfections. As seen in FIG. 24A, FIG. 24B, FIG. 24C, and FIG. 24D, αCD3 (hSP34) targeted LNPs resulted in GFP expression in both CD4 and CD8 T-cells while αCD8 (TRX2) targeted LNPs resulted in GFP expression selective in CD8 T-cells only as expected. Furthermore, non-binding (mutOKT8) control LNP resulted in no GFP expression in either cell type. Lipid 9 and 15 αCD3 (hSP34) targeted LNPs exhibited similar levels of GFP expression indicating that the two lipids are equally efficient in this cellular uptake pathway. However, in the CD8 binding and uptake pathway, αCD8 (TRX2) targeted Lipid 15 LNPs outperformed the corresponding Lipid 9 LNPs transfecting both a larger fraction of CD8 T-cells (expressed as % GFP+ cells) as well as more copies of GFP protein on a per cell basis (expressed as GFP MFI). As seen in FIG. 24E, FIG. 24F, FIG. 24G, and FIG. 24H, αCD3 (hSP34) targeted LNPs resulted in binding to both CD4 and CD8 T-cells while αCD8 (TRX2) targeted LNPs bound selectively to CD8 T-cells only (measure as DiI dye % and MFI in the two cell populations). Furthermore, non-binding (mutOKT8) control LNP resulted in no significant binding to either T-cell type as expected. Lipid 9 and 15 LNPs bound to similar fractions of CD4 and CD8 T-cells as indicated by similar % DiI+ cells in both populations. However slightly higher binding was observed with Lipid 15 LNPs in the CD8 binding and uptake pathway relative to Lipid 9 LNPs as indicated by brighter dye fluorescence intensity (DiI MFI values) on a per cell basis.

### Example 37. GFP Protein Expression in NK-Cells, Granulocytes, and B-Cells, with Lipid 9, 15, DLin-KC3-DMA Lipid αCD3 (Hsp34) Targeted LNPs in Human Whole Blood

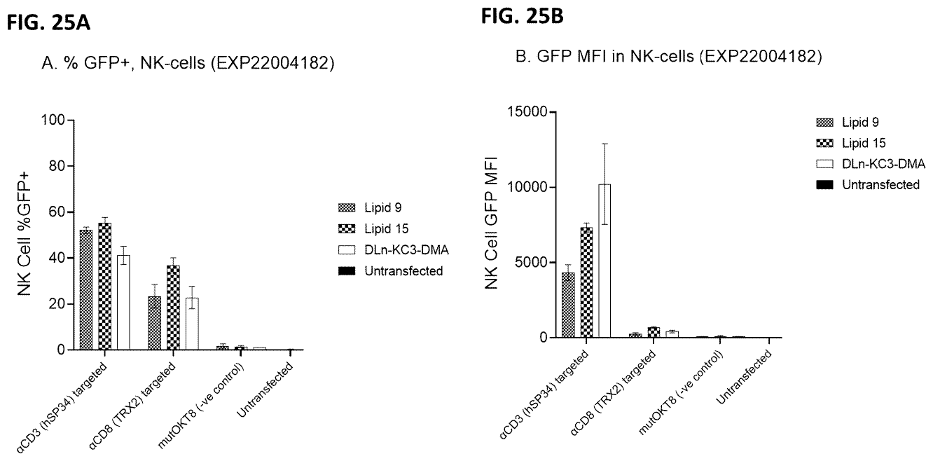

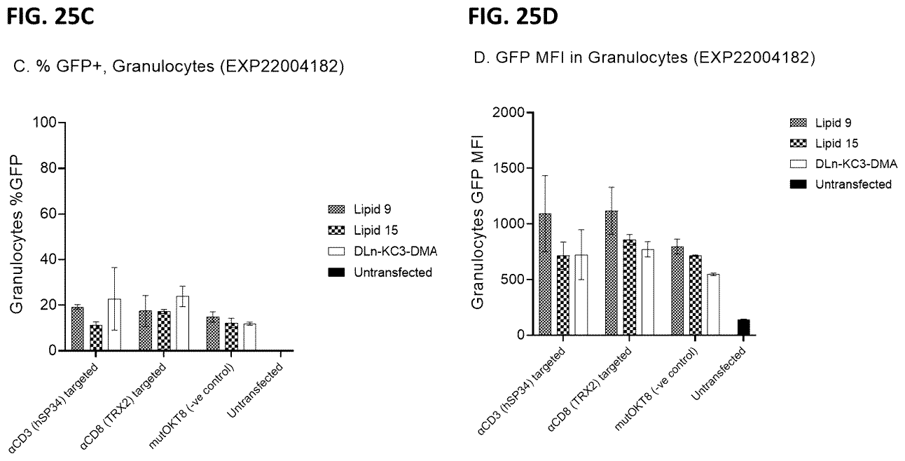

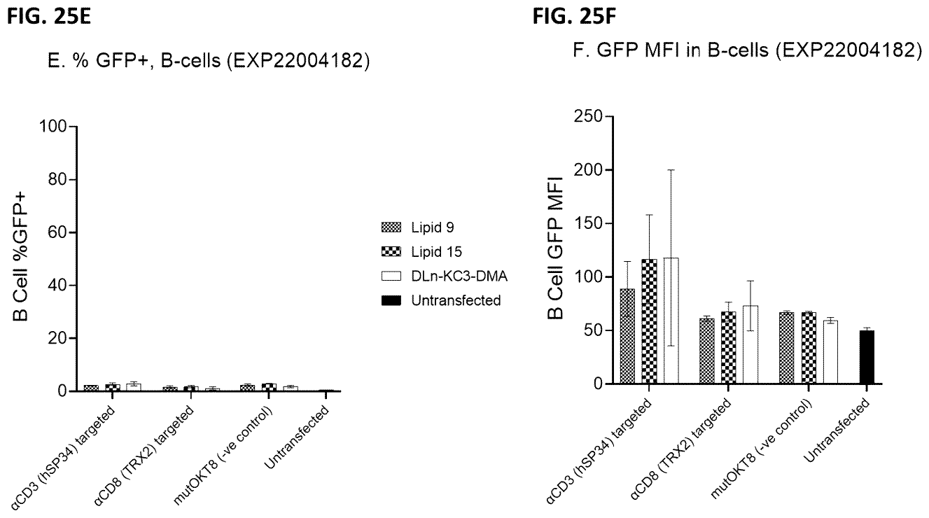

Lipid 9, 15 and DLin-KC3-DMAαCD3 and αCD8 targeted (and a non-binding Antibody, mutOKT8, as a negative control) GFP mRNA LNP transfected whole blood samples (of Example 38) were also analyzed for GFP expression in NK-cells, Granulocytes and B-cells using the protocol described in Example 21 for whole blood transfections. As seen in FIG. 25A and FIG. 25B, both αCD3 (hSP34) targeted LNPs and αCD8 (TRX2) targeted LNPs resulted in GFP expression in NK-cells as expected. Furthermore, no GFP expression was observed in NK-cells with the non-binding (mutOKT8) control LNP. Lipid 9 and 15 αCD3 (hSP34) targeted LNPs exhibited similar levels of GFP expression in NK-cells indicating that the two lipids are equally efficient in this cellular uptake pathway. However, in the CD8 binding and uptake pathway, αCD8 (TRX2) targeted Lipid 15 LNPs outperformed the corresponding Lipid 9 LNPs transfecting both a larger fraction of NK-cells (expressed as % GFP+ cells) as well as more copies of GFP protein on a per cell basis (expressed as GFP MFI). As seen in FIG. 25C, FIG. 25D, FIG. 25E, and FIG. 25F, neither targeting modalities resulted in any significant GFP expression in Granulocytes and B-cells.

### Example 38. LNP Association (Measured as DiI-Dye Fluorescence) in NK-Cells, Granulocytes, and B-Cells with Lipid 9, 15, DLin-KC3-DMA Lipid αCD3 (Hsp34) Targeted LNPs in Human Whole Blood

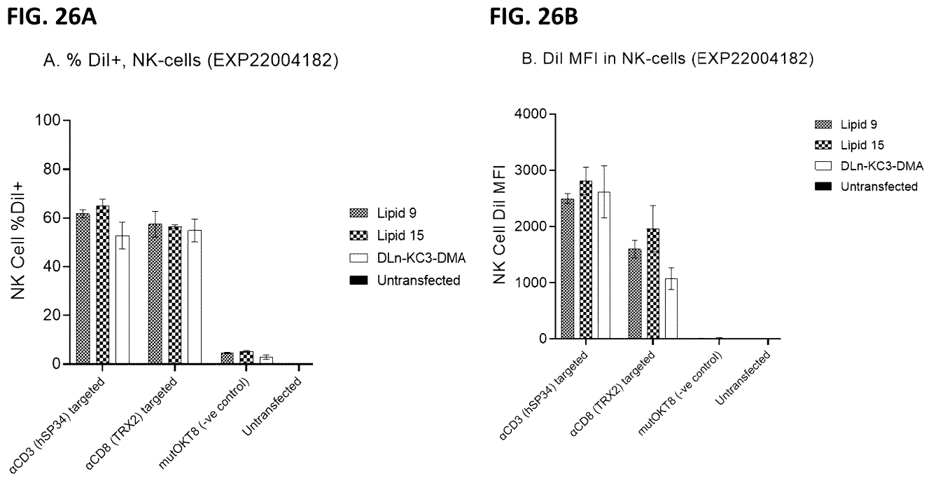

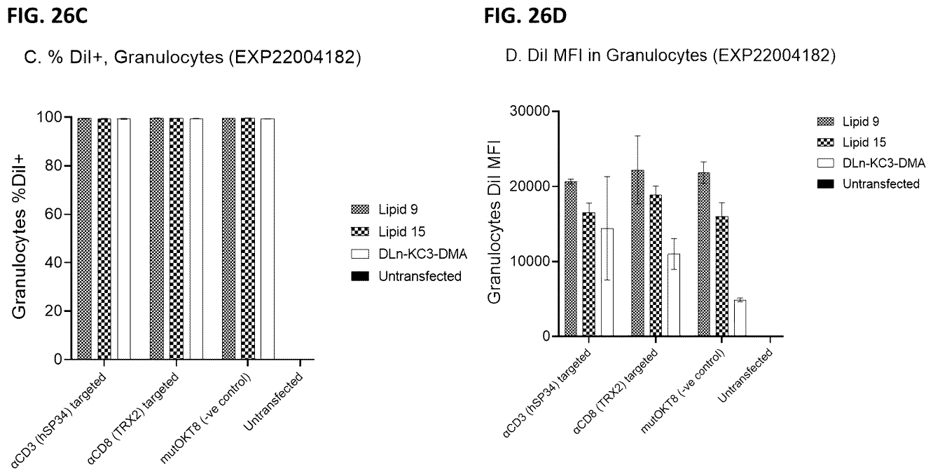

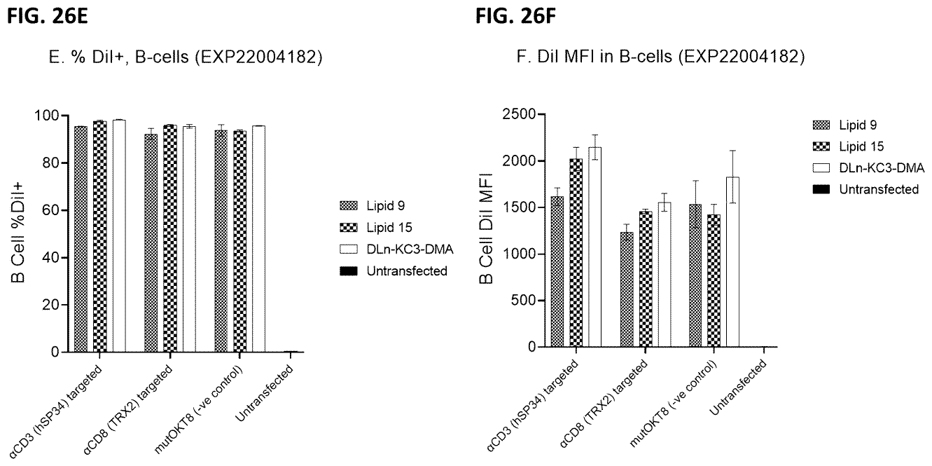

Lipid 9, 15 and DLin-KC3-DMAαCD3 and αCD8 targeted (and a non-binding Antibody, mutOKT8, as a negative control) GFP mRNA LNP transfected whole blood samples (of Example 36) were also analyzed for binding to NK-cells, Granulocytes and B-cells using the protocol described in Example 23 for whole blood transfections. As seen in FIG. 26A and FIG. 26B, both αCD3 (hSP34) targeted LNPs and αCD8 (TRX2) targeted LNPs bound to NK-cells as expected. Furthermore, the non-binding (mutOKT8) control LNP did not result in any significant bind to NK-cells as expected. As seen in FIG. 26C, FIG. 26D, FIG. 26E, and FIG. 26F, both targeting modalities resulted in non-specific binding to Granulocytes and B-cells however as reported in Example 39 above, no GFP expression was observed indicating that RNA was not delivered to the cytosol of either cell type.

### Example 39. In Vitro CAR (TTR-023) and mCherry Expression in Primary Human T-Cells Transfected with aCD8 (TRX2) Targeted Lipid 9 and DLin-KC3-DMA LNPs

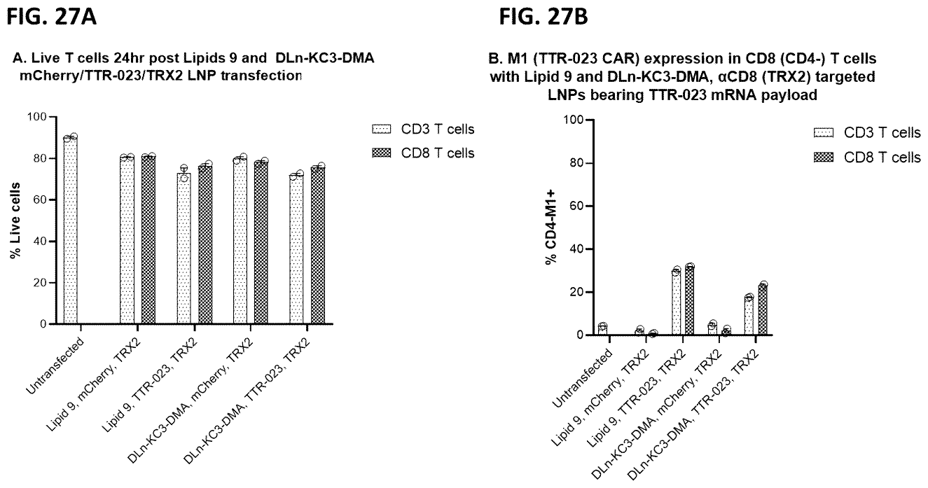

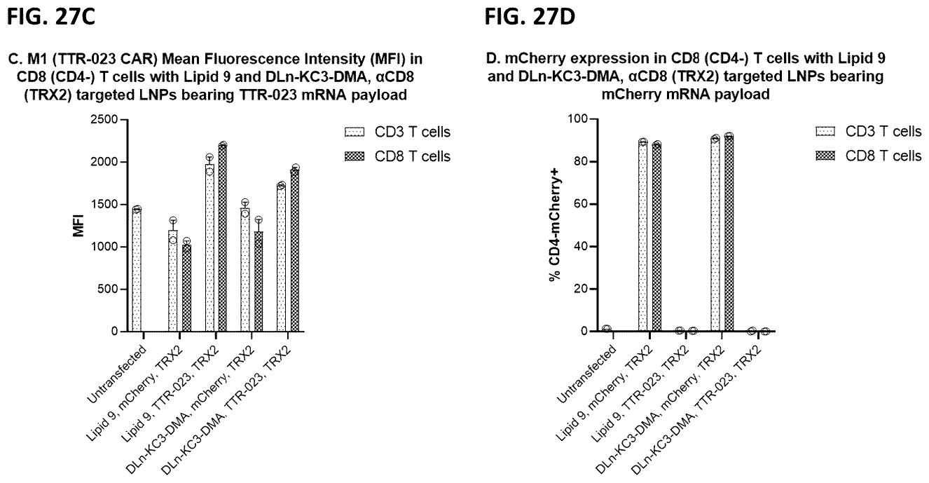

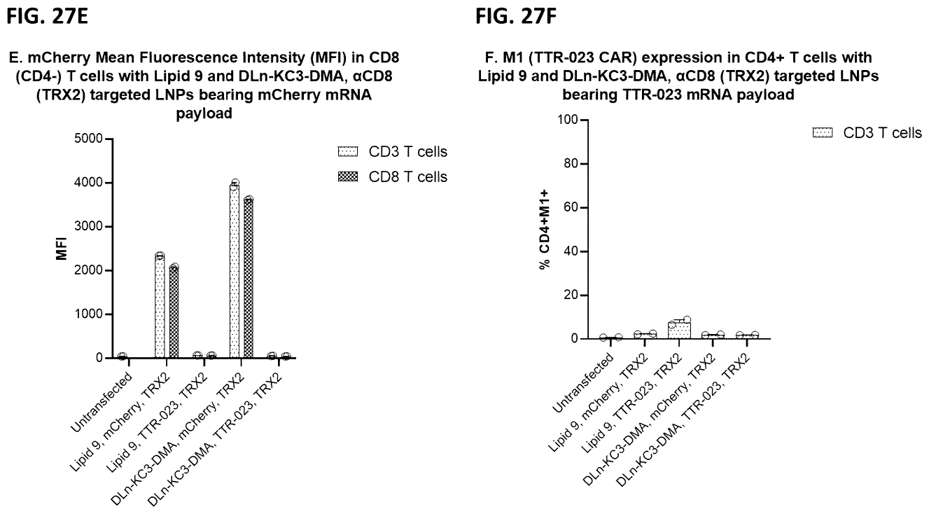

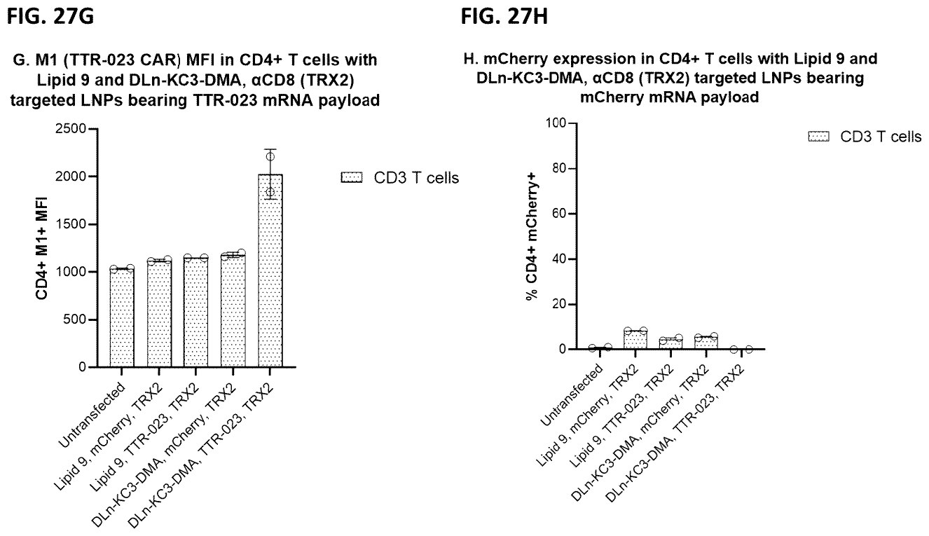

This example compares reporter protein (mCherry) and αCD20 CAR (TTR-023) protein expression resulting from LNP's derived from Lipid 9 to comparator Lipid DLin-KC3-DMA. Nanoparticles bearing mCherry mRNA or an αCD20 CAR encoding mRNA (TTR-023) were produced using the microfluidic mixing and buffer exchange processes described in Example 2. An αCD8 Fab-conjugate (TRX2) was incorporated into the parent LNPs to obtain the final Antibody targeted LNP formulation using the process described in Example 5. Particles thus produced were tested in vitro in primary human CD3+ T cells to assess protein (mCherry or CAR) expression via mCherry fluorescence or detection of an M1 tag on the extracellular domain of the TTR-023 transmembrane CAR protein, respectively. Transfected cells were gated as CD4+(CD4 population) and CD4− (CD8 population) and levels of protein expression monitored (as % M1+ and M1 MFI for CAR expression or reporter protein fluorescence for mCherry expression) in the two populations were used to assess specificity in the CD8 population with this αCD8 (TRX2) targeting strategy. Protein expression was selectively observed in the CD4− population (CD8 cells, as illustrated by the M1% and M1 MFI values in FIG. 27B and FIG. 27C versus F and G or mCherry % and mCherry MFI values in FIG. 27D and FIG. 27E versus FIG. 27G and FIG. 27H) with both lipid 9 and comparator lipid DLin-KC3-DMA formulations confirming that the TRX2 antibody enables receptor mediated uptake specifically into CD8 T-cells. Lipid 9 LNPs outperformed DLin-KC3-DMA LNPs in CAR expression levels while DLin-KC3-DMA LNPs outperformed Lipid 9 LNPs in mCherry expression levels suggesting that different optimal lipid compositions may be required for expression of intracellular proteins versus membrane bound proteins. Both TRX2 targeted lipid formulations with either CAR or mCherry payloads were well tolerated by primary human T-cells at the 1 ug/mL per 500,000 T-cells dose level (FIG. 27A).

### Example 40. In Vitro CAR-T Cell Function by Raji (B-Cell) Co-Culture with aCD20 Car (TTR-023) Expressing T-Cells Derived by Transfection of Primary Human T-Cells (of Example 27) with aCD8 (TRX2) Targeted Lipid 9 and DLin-KC3-DMA LNPs Bearing Car-mRNA or Mcherry-mRNA (as Negative Control)

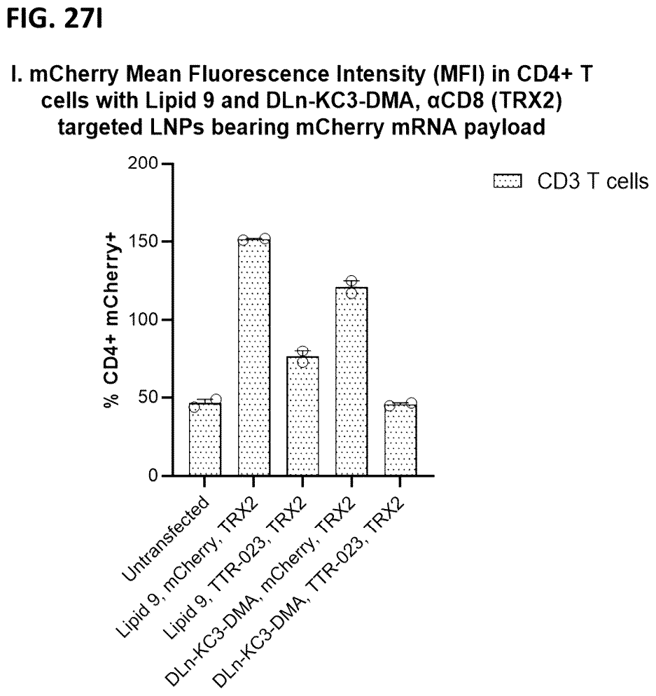

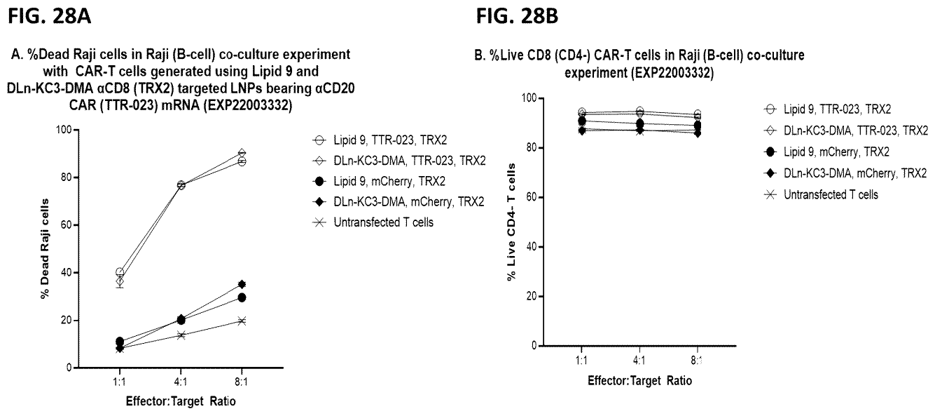

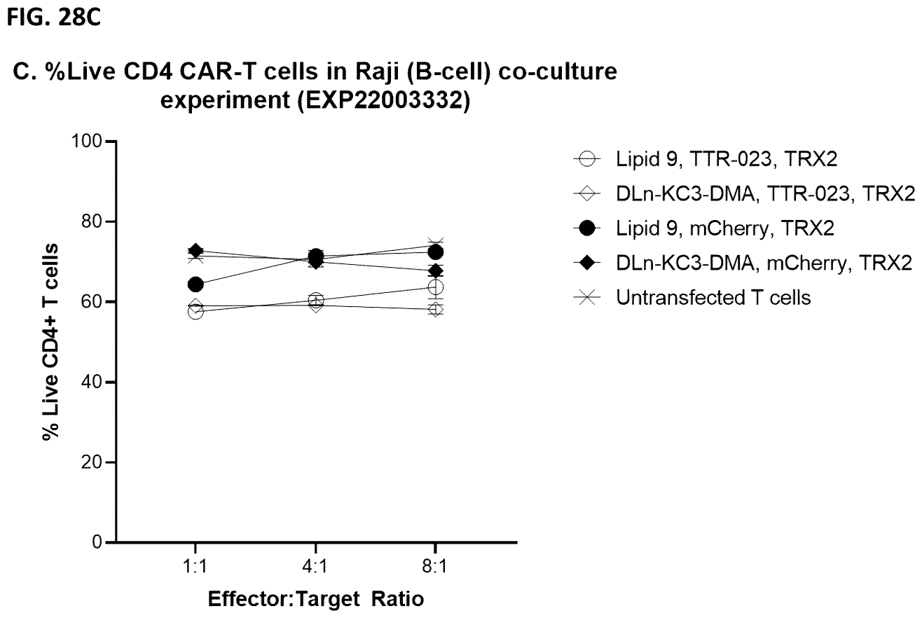

CAR-T cells produced in Example 39 were co-cultured with Raji (B-cells) at Effector:Target (E:T) (T-cell:B-cell) ratios of 1:1, 4:1, and 8:1 for a period of 24 hours and the fraction of Live B-cells and T-cells measured using the protocol described in Example 20. As seen in FIG. 28A, the fraction of dead B-cells increased at higher E:T ratios in a dose-dependent manner. Furthermore, T-cells expressing TTR-023 CAR protein exhibited significantly higher cytotoxicity towards B-cells relative to mCherry transfected T-cells as indicated by a 4× higher % of dead Raji cells at the three E:T ratios tested. This suggests that CAR engagement to target cell CD20 receptor and the downstream target specific granzyme perforin apoptotic pathway plays a major role in the observed T-cell activity while T-cell activation (possibly resulting for CD8 receptor engagement by the TRX2 antibody) over background levels of T-cell cytoxicity towards B-cells is a minor contributor in the overall activity of the CAR-T cells observed here. Both Lipid 9 and DLin-KC3-DMA LNP formulations were equally cytotoxic towards B-cells and both formulations were well tolerated by the CD4 and CD8 T-cells in the co-culture experiment with T-cell viability values remaining either slightly below (CD4 cells) or slightly above (CD4−, CD8 T-cells) the un-transfected controls as seen FIG. 28B and FIG. 28C, respectively.

### Example 41. In Vitro CAR (TTR-023) and mCherry Expression in Primary Human T-Cells Transfected with aCD8 (TRX2) Targeted Lipid 15 and DLin-KC3-DMA LNPs

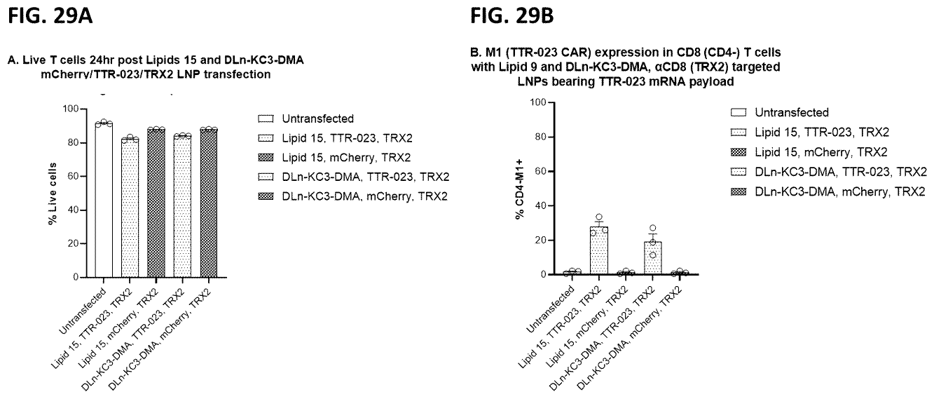

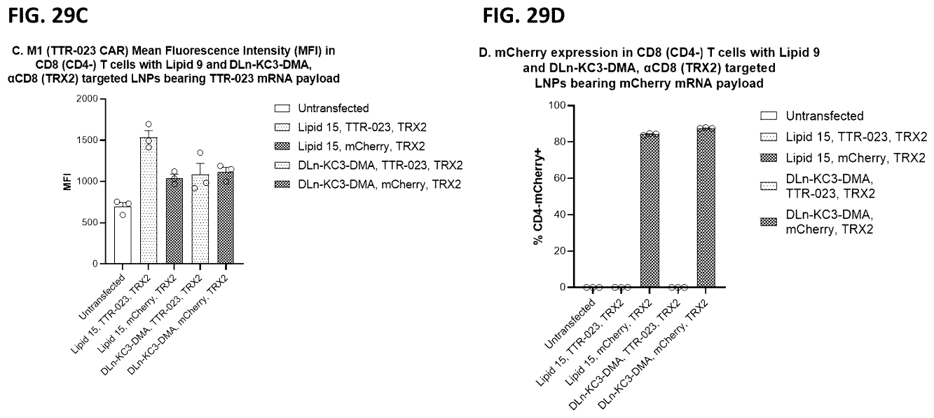

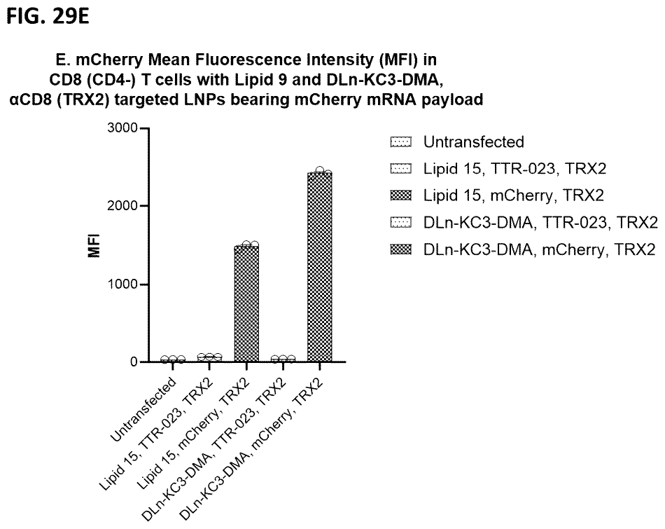

This example compares reporter protein (mCherry) and αCD20 CAR (TTR-023) protein expression resulting from LNP's derived from Lipid 15 to comparator Lipid DLin-KC3-DMA. Nanoparticles bearing mCherry mRNA or an αCD20 CAR encoding mRNA (TTR-023) were produced using the microfluidic mixing and buffer exchange processes described in Examples 2 and 6. An αCD8 Fab-conjugate (TRX2) was incorporated into the parent LNPs to obtain the final Antibody targeted LNP formulation using the process described in Example 5. Particles thus produced were tested in vitro in primary human CD3+ T cells to assess protein (mCherry or CAR) expression via mCherry fluorescence or detection of an M1 tag on the extracellular domain of the TTR-023 transmembrane CAR protein, respectively. Transfected cells were gated as CD4+(CD4 population) and CD4− (CD8 population) and levels of protein expression monitored (as % M1+ and M1 MFI for CAR expression or reporter protein fluorescence for mCherry expression) in the two populations. Lipid 15 LNPs outperformed DLin-KC3-DMA LNPs in CAR expression levels (FIG. 29B and FIG. 29C) while DLin-KC3-DMA LNPs outperformed Lipid 15 LNPs in mCherry expression levels (FIG. 29D and FIG. 29E) suggesting that different optimal lipid compositions may be required for expression of intracellular proteins versus membrane bound proteins. Both TRX2 targeted lipid formulations with either CAR or mCherry payloads were well tolerated by primary human T-cells (FIG. 29A).

### Example 42. In Vitro Car-T Cell Function by Raji (B-Cell) Co-Culture with aCD20 Car (TTR-023) Expressing T-Cells Derived by Transfection of Primary Human T-Cells (of Example 29) with aCD8 (TRX2) Targeted Lipid 15 and DLin-KC3-DMA LNPs Bearing Car-mRNA or Mcherry-mRNA (as Negative Control), Bite (as Positive Control)

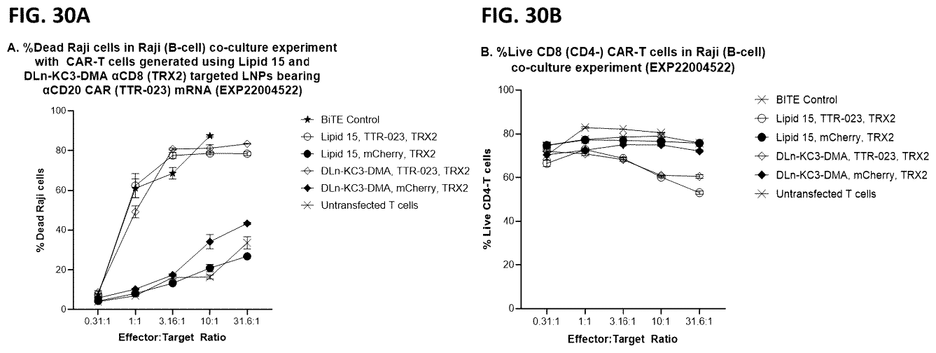

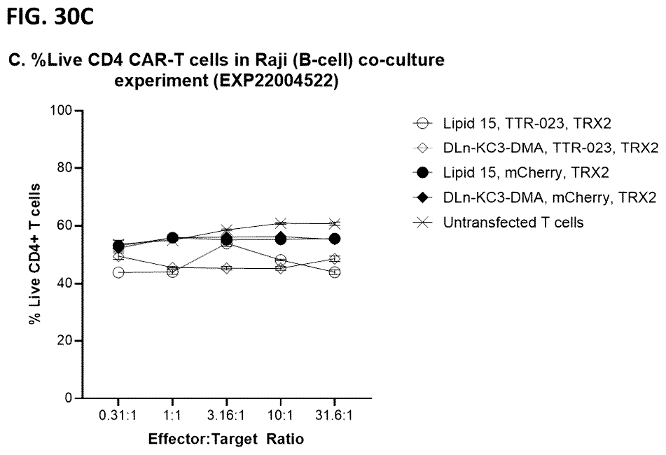

CAR-T cells produced in Example 40 were co-cultured with Raji (B-cells) at Effector:Target (E:T) (T-cell:B-cell) ratios of 0.31:1, 1:1, 3.16:1, 10:1, and 31.6:1 for a period of 24 hours and the fraction of Live B-cells and T-cells measured using the protocol described in Example 20. As seen in FIG. 30A, the fraction of dead B-cells increased at higher E:T ratios in a dose dependent manner up to an E:T of 3.16:1 and plateaued at E:T ratios indicating strong cytotoxic activity at an E:T of 3.16:1. Furthermore, T-cells expressing TTR-023 CAR protein exhibited significantly higher cytotoxicity towards B-cells relative to mCherry transfected T-cells as indicated by a 4× higher % of dead Raji cells for E:T ratios of 3.16 and below. This suggests that CAR engagement to target cell CD20 receptor and the downstream target specific granzyme perforin apoptotic pathway plays a major role in the observed T-cell activity while T-cell activation (possibly resulting for CD8 receptor engagement by the TRX2 antibody) over background levels of T-cell cytoxicity towards B-cells is a minor contributor in the overall activity of the CAR-T cells observed. Both Lipid 15 and DLin-KC3-DMA LNP formulations were equally cytotoxic towards B-cells exhibiting similar activity to the Bi-specific B-cell Engager (BiTE, bispecific antibody) positive control as seen in FIG. 30A. Both Lipid 15 and DLin-KC3-DMA formulations were well tolerated up to an E:T ratio of 3.16:1 with lower T-cell viability values observed in the CD8 T-cell population at the higher E:T ratios of 10:1 and 31.6:1 as seen FIG. 30B and FIG. 30C, respectively.

### Example 43. In Vitro Protein Expression (GFP) and LNP Association (Measured as DiI-Dye Fluorescence) in Primary Human T-Cells of aCD3 Targeted Lipids 15, 9, 10, and 13 and Comparator (DLin-KC3-DMA) LNPs Stored at 4° C. and after 1 Freeze-Thaw Cycle (Post −80° C. Storage)

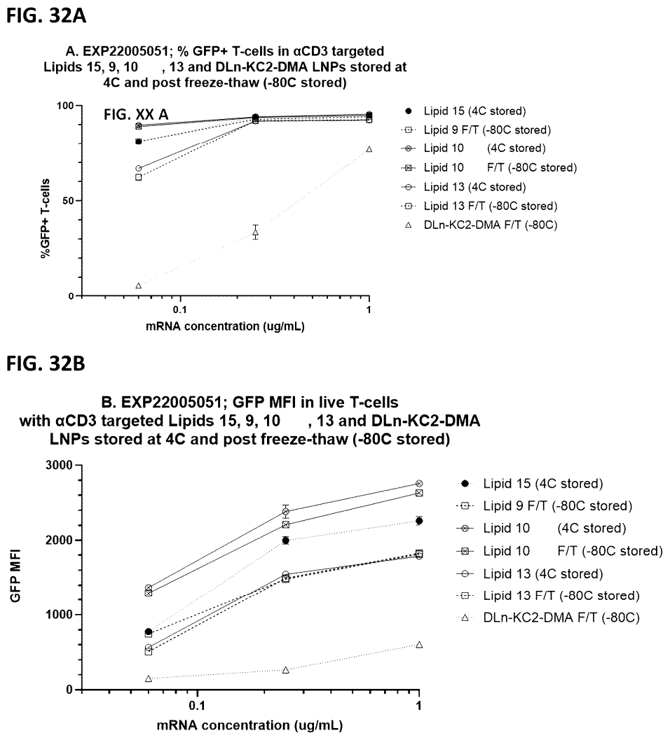

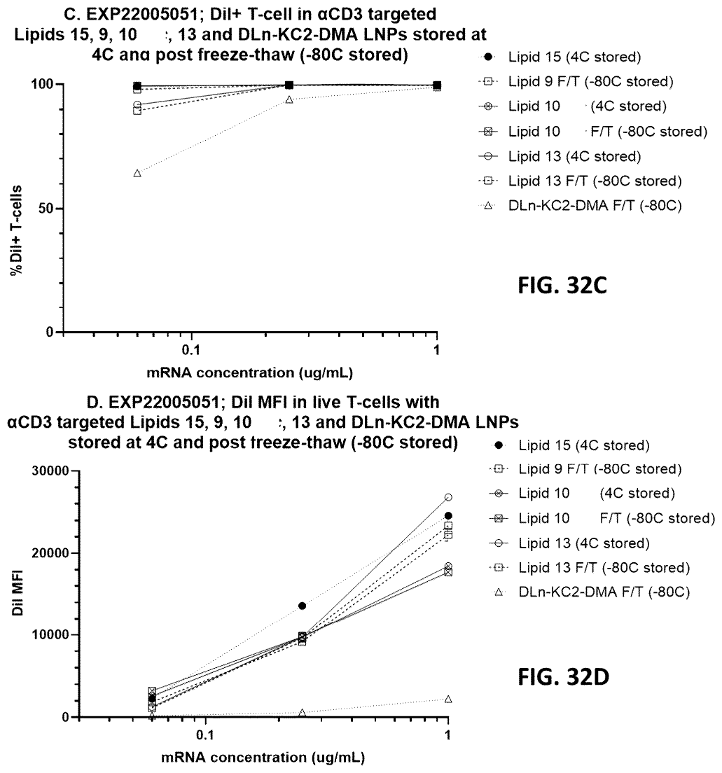

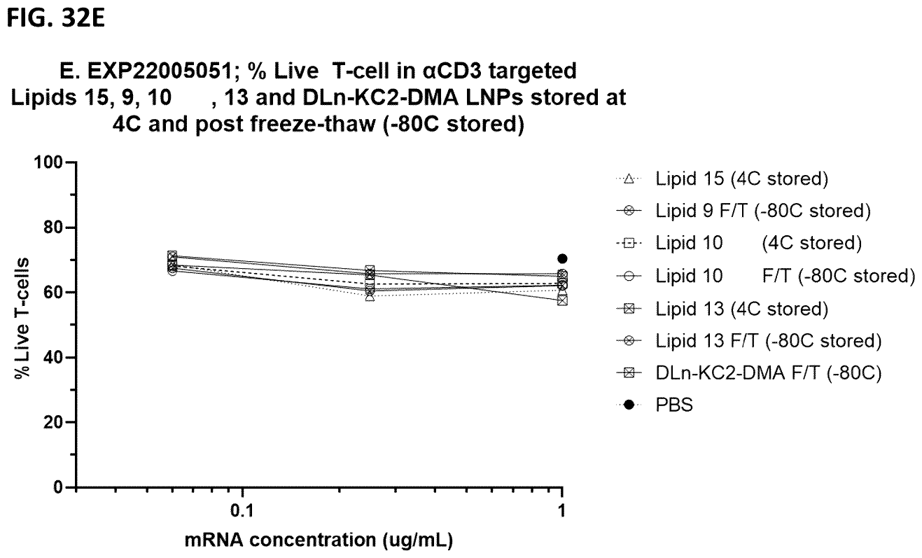

This example compares the GFP protein expression resulting from LNP's derived from Lipids 15, 9, 10 and comparator lipid DLin-KC2-DMA LNPs. Nanoparticles bearing GFP encoding mRNA (and optionally a fluorescent dye label (DiI-C18-5DS)) were produced using the microfluidic mixing and buffer exchange processes described in Example 2 and 6. An αCD3 Fab-conjugate was incorporated into the parent LNPs to obtain the final Antibody targeted LNP formulation using the process described in Example 5. Particles thus produced were tested in vitro in primary human CD3+ T cells to assess reported gene expression. As shown in FIG. 32A and FIG. 32B, at all dose levels, Lipid 15 and 10 LNPs performed similarly both in terms of fraction of GFP+ T-cells (reflected by % GFP+ cells) and the copies of GFP protein produced on a per cell basis (reflected by GFP MFI values) and significant better relative to Lipid 9 LNPs with respect to the copies of GFP protein produced on a per cell basis (reflected by GFP MFI values) particularly at the lower dose levels. Thus, improvements over lipid 9 performance could be achieved either by modification of the O-acyl substituent (from Linoleoyl of Lipid 9 to Oleoyl of Lipid 15) or by modification of N-acyl substituent (from the succinic acid derived 14 carbon N-acyl substituent of Lipid 9 to the succinic acid derived 12 carbon N-acyl substituent of Lipid 10). Additionally, as seen in FIGS. 32C and D, Lipid 9 and Lipid 13 LNPs exhibited similar cell association levels (both in terms of fraction of DiI+ cells and in terms of the copies of cell associated LNPs reflected by DiI MFI values shown in FIG. 32C and FIG. 32D) at all dose levels, however, Lipid 9 outperformed Lipid 13 LNPs in terms of protein expression both in terms of fraction of GFP+ T-cells (reflected by % GFP+ cells) and the copies of GFP protein produced on a per cell basis (reflected by GFP MFI values) suggesting inferior endosomal escape capabilities of Lipid 13 LNPs relative to Lipid 9 LNPs. The performance of Lipids 10 and 13 LNPs was well preserved after 1 Freeze Thaw cycle (Post −80° C. storage) as illustrated by comparison of % GFP+ cells and GFP MFI values before (4° C. stored) and after −80° C. storage as seen in FIG. 32A and FIG. 32B.

### Example 44. In Vivo T-Cell Reprogramming Using GFP Reporter Protein and α-CD8 (TRX-2) Targeted LNPs Based on Lipids 9, 15 and Comparator Lipid DLin-KC3-DMA (and Formulated with 1.5 Mol % Dpg-Peg) in Human T-Cell Engrafted Nsg Mice

NSG Mice were dosed using protocols described in Example 22. Plasma samples were drawn immediately prior to sacrificing the animals and spleen and liver harvested for analysis 24 hours post injection following the study design shown in Table 25.

TABLE 25
NSG Mice GFP T-Cell Reprogramming study design

| | Mice | Formulation | | Targeting |
|---|---|---|---|---|
| Groups | # | Dosed | Payload | Antibody |
| 1 | 2 | Buffer Control | NA | NA |
| 2 | 4 | Lipid 15; 1.5% | GFP-mRNA | TRX-2 |
| | | DPG-PEG; DiI | | |
| | | dye labelled | | |
| 3 | 4 | DLin-KC3-DMA; | GFP-mRNA | TRX-2 |
| | | 1.5% DPG-PEG; | | |
| | | No dye label used | | |
| 4 | 3 | Lipid 9; 1.5% | GFP-mRNA | TRX-2 |
| | | DPG-PEG; DiI | | |
| | | dye labelled | | |

CD4 and CD8 T-cells were stained sorted (by FACS) in blood, spleen and liver samples, liver samples were additionally stained and sorted for hepatocytes, endothelial cells, Kupffer cells, mouse macrophages and mouse myeloid cells using the Flow Panel depicted in Table 26 and Table 27. GFP fluorescence and DiI Dye fluorescence was used for quantitation of GFP protein expression (expressed as % GFP+ Cells and Mean Fluorescence Intensity (MFI) of GFP+ Cells) and LNP association (via DiI label fluorescence, expressed as % DiI+ cells and DiI-MFI of DiI+ Cells) in the stated cell types of interest.

TABLE 26
Cell Markers (Dead-Live, LNP, Protein, HuCD45, huCD3, huCD4) and
Fluorophores used

| Markers | Dead live stain | LNP (Dil dye) | GFP-Protein | huCD45 | huCD3 | huCD4 |
|---|---|---|---|---|---|---|
| Fluorophore | e-flour780 | APC | Green | BUV395 | BUV805 | BV711 |
| | | | Fluorescence | | | |

TABLE 27
Cell markers (Dead-Live, huCD8, muCD45, hu/muCD11b, muCD31, F4/80) and
Fluorophores used

| Markers | Dead live stain | huCD8 | muCD45 | Hu/muCD11b+ | muCD31 | F4/80 |
|---|---|---|---|---|---|---|
| Fluorophore | e-flour780 | BV421 | BB700 | BV785 | BUV737 | PE Dazzle |

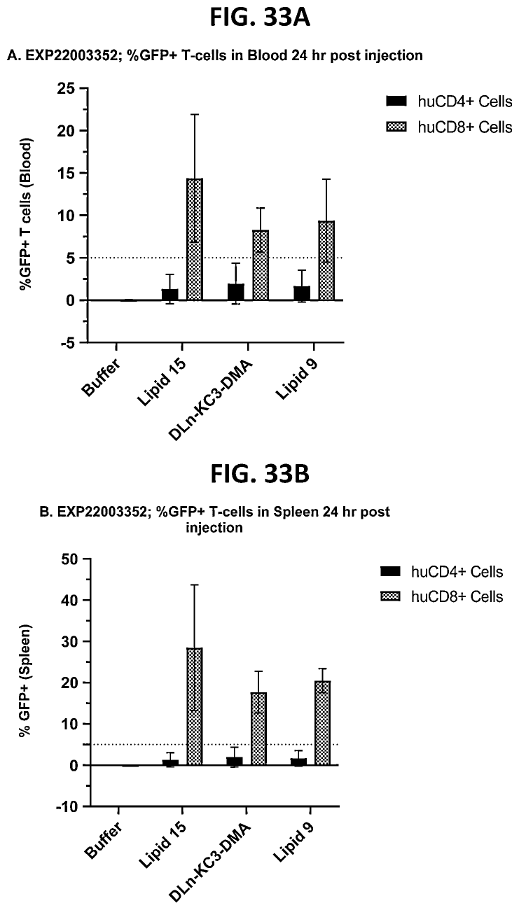

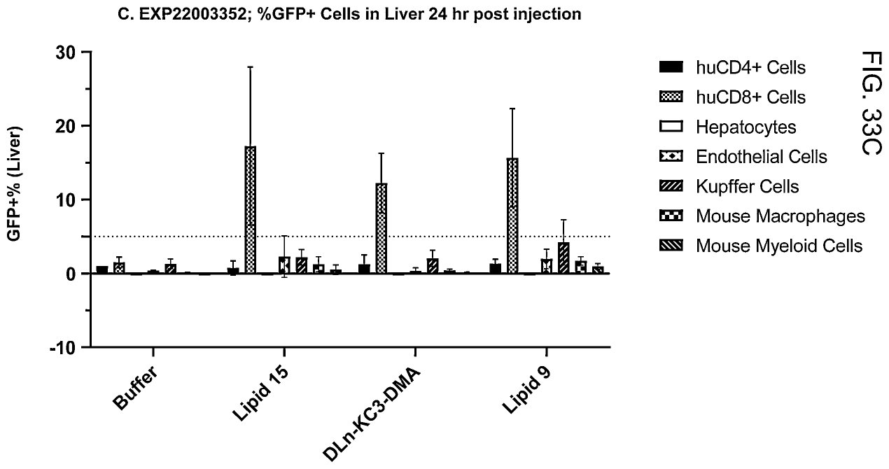

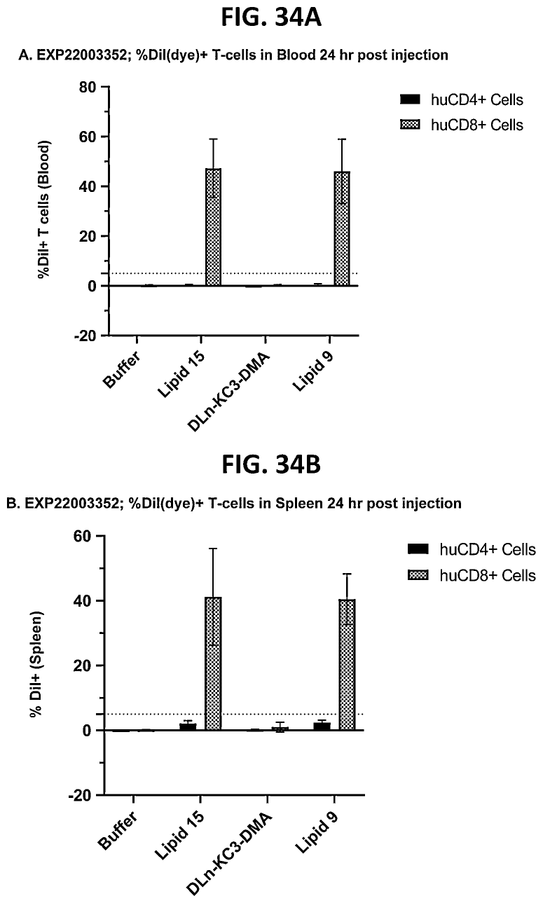

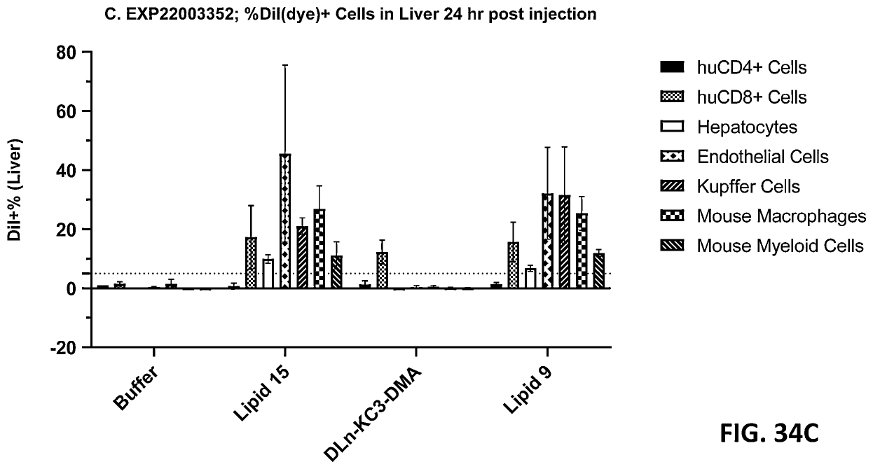

As seen in FIGS. 33A, 33B, and 33C, 7-25% GFP+ cells were detected in the CD8 T-cell population in blood, spleen and liver samples, while <3% of the CD4+ T-cells were GFP+ confirming differential in vivo reprogramming of the CD8+ T-cell population and LNP targeting using TRX-2 αCD8 antibody. As seen in FIGS. 34A, 34B, and 34C, LNP association was specific to the CD8 T-cell population in blood and spleen samples, however, significant levels of association to the CD4 population as well as endothelial cells, Kupffer Cells, and mouse macrophages was observed in the liver samples. Notably, despite non-specific LNP association, no GFP protein was detected (FIG. 33C) indicating that off target LNP association does not result in off-target mRNA delivery and protein expression.

### Example 45. In Vitro Protein Expression (GFP) and LNP Association (Measured as DiI-Dye Fluorescence) in Primary Human T-Cells of aCD2, aCD4, aCD7, CD28, TCR, and Non-Binding (Mutated OKT8) Targeted Fabs and Nanobodies mRNA Titration Along with aCD8 (TRX2 and 15C01) and aCD3(Hsp34) Antibody Targeted LNPs in Comparison of Lipids 15 and DLin-KC3-DMA

This example compares the GFP protein expression resulting from LNP's derived from Lipids 15, and lipid DLin-KC3-DMA LNPs with αCD2, αCD4, αCD7, αCD28, TCR, and non-binding (mutated OKT8) as comparison of αCD8 and αCD3 Target.

Nanoparticles bearing GFP encoding mRNA and a fluorescent dye label (DiI-C18-5DS) were produced using the microfluidic mixing and buffer exchange processes described in Example 2. Fab-lipid conjugates generated from methods described in Example 4 while generation of Nb-conjugated differed in using 1:1:4 Nb:DSPE-3.4K PEG-maleimide:DSPE-2K PEG-OCH3 and a 50 kD UF membrane for separation of Nb-conjugate from free Nb. Fab-conjugate and Nb-conjugate were incorporated into the parent LNPs based on the optimal Fab and Nb density (Table 28) to obtain the final Antibody targeted LNP formulation using the process described in Example 5. Particles thus produced were tested in vitro in primary human CD3+ T cells to assess reported gene expression at approximately 2.5 ug/mL, 0.5 ug/mL, and 0.1 ug/mL mRNA for approximately 24 hours. Levels of transfection of both CD8 and CD4 cells was measured by flow cytometry.

TABLE 28
Antibody Insertion Conditions

| | | | Conjugate | | |
|---|---|---|---|---|---|
| | | | Insertion | | |
| | | | Density (g | Ionizable | Insertion |
| Sample ID | Target | Clone | Ab/mol lipid) | Lipid | Condition |
| 221101EYS-1-1 | aCD2 | 9.6 Fab NoDS | 3 | Lipid 15 | 37° C. for 4 hours in |
| | | | | | pH 6.5 MBS |
| 221101EYS-1-4 | aCD2 | TS2/18.1 fab NoDS | 3 | Lipid 15 | 37° C. for 4 hours in |
| | | | | | pH 6.5 MBS |
| 221101EYS-1-7 | TCR | T017000700 Nb | 2.7 | Lipid 15 | 37° C. for 4 hours in |
| | | | | | pH 6.5 MBS |
| 221101EYS-1-10 | aCD4 | Ibalizumab Fab | 18.4 | Lipid 15 | 37° C. for 4 hours in |
| | | NoDS | | | pH 6.5 MBS |
| 221101EYS-1-13 | aCD4 | hBF5 Fab bDS | 1.5 | Lipid 15 | 37° C. for 4 hours in |
| | | | | | pH 6.5 MBS |
| 221101EYS-1-16 | aCD4 | T023200008 Nb | 0.93 | Lipid 15 | 37° C. for 4 hours in |
| | | | | | pH 6.5 MBS |
| 221101EYS-1-19 | aCD7 | V1 Nb | 2.8 | Lipid 15 | 37°C for 4 hours in |
| | | | | | pH 6.5 MBS |
| 221101EYS-1-22 | aCD28 | Hz511.A1 Fab bDS | 1.5 | Lipid 15 | 37° C. for 4 hours in |
| | | | | | pH 6.5 MBS |
| 221101EYS-1-25 | aCD28 | 28CD065G01 Nb | 0.9 | Lipid 15 | 37° C. for 4 hours in |
| | | | | | pH 6.5 MBS |
| 221101EYS-1-28 | aCD8 | T0347015C01 Nb | 2 | Lipid 15 | 37° C. for 4 hours in |
| | | | | | pH 6.5 MBS |
| 221101EYS-1-31 | aCD8 | A044300805_V8 | 5.5 | Lipid 15 | 37°C for 4 hours in |
| | | Nb | | | pH 6.5 MBBS |
| 221101EYS-1-34 | aCD3 | hSP34 Fab NoDS | 9 | Lipid 15 | 37° C. for 4 hours in |
| | | | | | pH 6.5 MBS |
| 221101EYS-1-37 | aCD8 | TRX2 Fab bDS | 9 | Lipid 15 | 37° C. for 4 hours in |
| | | | | | pH 6.5 MBS |
| 221101EYS-1-40 | Negative | mutOKT8 Fab | 9 | Lipid 15 | 37° C. for 4 hours in |
| | Control | NoDS | | | pH 6.5 MBS |
| 221101EYS-2-1 | aCD2 | 9.6 Fab NoDS | 3 | DLIN-KC3- | 60° C. for 1 hour in |
| | | | | DMA | pH 7.4 HBS |
| 221101EYS-2-4 | aCD2 | TS2/18.1 fab NoDS | 3 | DLIN-KC3- | 60° C. for 1 hour in |
| | | | | DMA | pH 7.4 HBS |
| 221101EYS-2-7 | TCR | T017000700 Nb | 2.7 | DLIN-KC3- | 60° C. for 1 hour in |
| | | | | DMA | pH 7.4 HBS |
| 221101EYS-2-10 | aCD4 | Ibalizumab Fab | 18.4 | DLIN-KC3- | 60° C. for 1 hour in |
| | | NoDS | | DMA | pH 7.4 HBS |
| 221101EYS-2-13 | aCD4 | hBF5 Fab bDS | 1.5 | DLIN-KC3- | 60° C. for 1 hour in |
| | | | | DMA | pH 7.4 HBS |
| 221101EYS-2-16 | aCD4 | T023200008 Nb | 0.93 | DLIN-KC3- | 60° C. for 1 hour in |
| | | | | DMA | pH 7.4 HBS |
| 221101EYS-2-19 | aCD7 | V1 Nb | 2.8 | DLIN-KC3- | 60° C. for 1 hour in |
| | | | | DMA | pH 7.4 HBS |
| 221101EYS-2-22 | aCD28 | Hz511.A1 Fab bDS | 1.5 | DLIN-KC3- | 60° C. for 1 hour in |
| | | | | DMA | pH 7.4 HBS |
| 221101EYS-2-25 | aCD28 | 28CD065G01 Nb | 0.9 | DLIN-KC3- | 60° C. for 1 hour in |
| | | | | DMA | pH 7.4 HBS |
| 221101EYS-2-28 | aCD8 | T0347015C01 Nb | 2 | DLIN-KC3- | 60° C. for 1 hour in |
| | | | | DMA | pH 7.4 HBS |
| 221101EYS-2-31 | aCD8 | A044300805_V8 | 5.5 | DLIN-KC3- | 60° C. for 1 hour in |
| | | Nb | | DMA | pH 7.4 HBS |
| 221101EYS-2-34 | aCD3 | hSP34 Fab NoDS | 9 | DLIN-KC3- | 60° C. for 1 hour in |
| | | | | DMA | pH 7.4 HBS |
| 221101EYS-2-37 | aCD8 | TRX2 Fab bDS | 9 | DLIN-KC3- | 60° C. for 1 hour in |
| | | | | DMA | pH 7.4 HBS |
| 221101EYS-2-40 | Negative | mutOKT8 Fab | 9 | DLIN-KC3- | 60° C. for 1 hour in |
| | Control | NoDS | | DMA | pH 7.4 HBS |

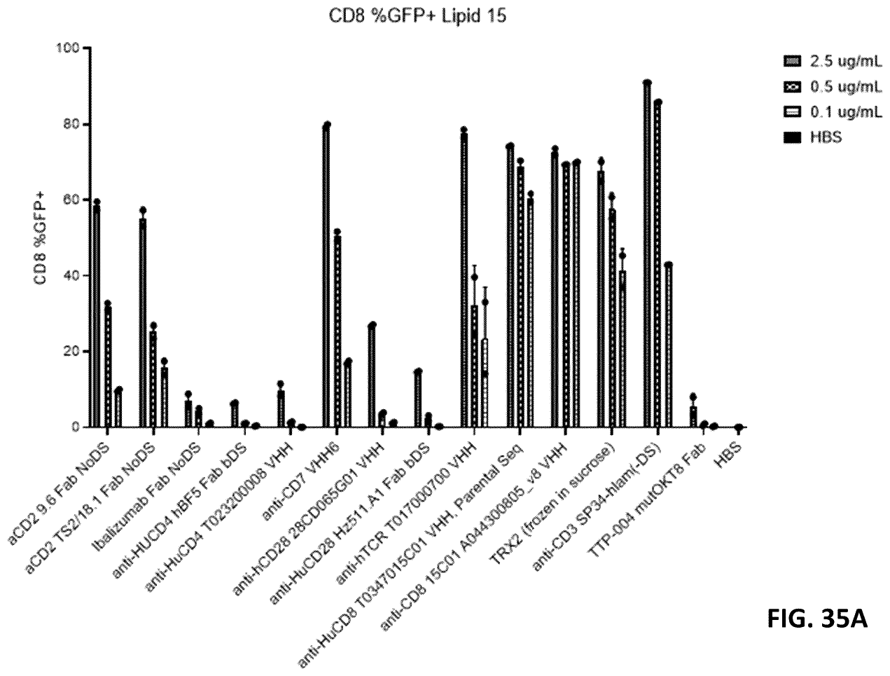

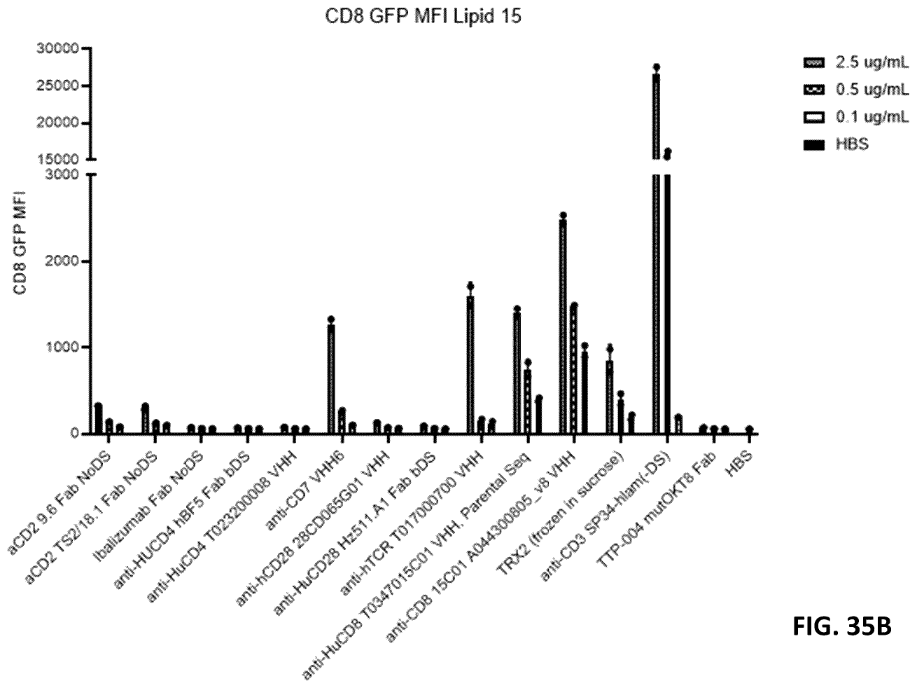

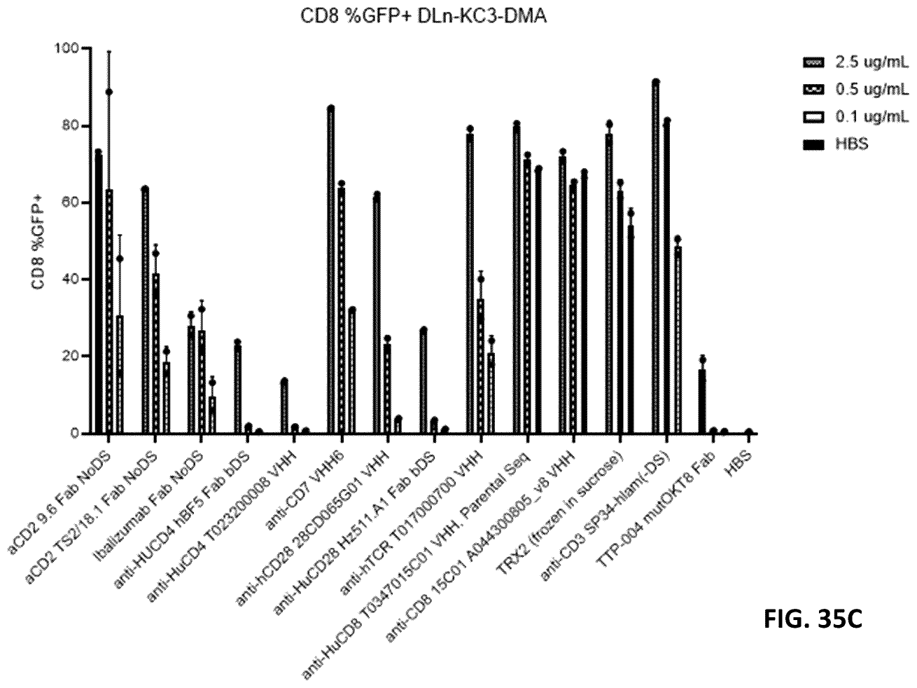

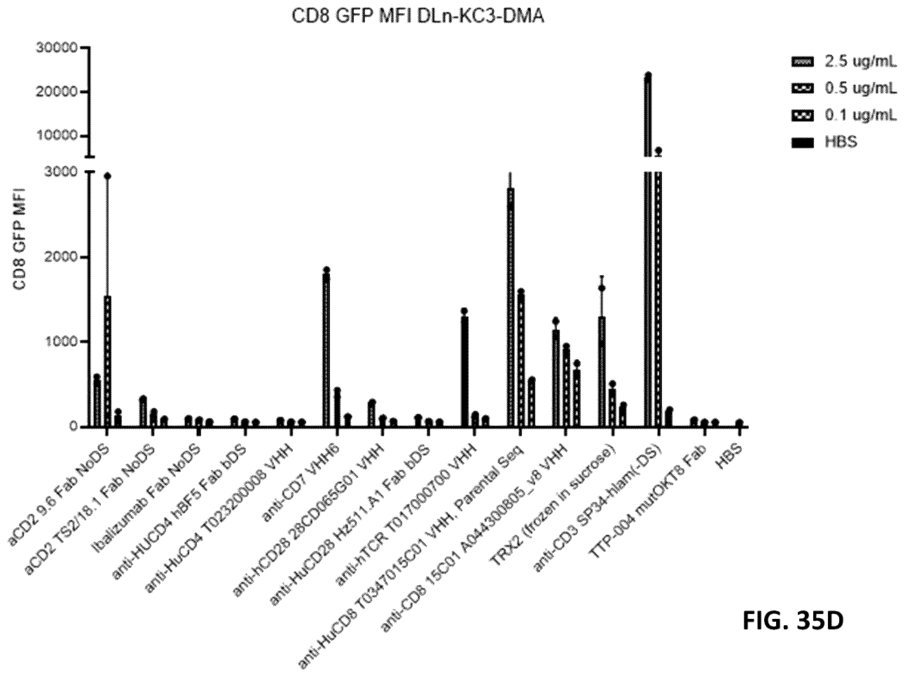

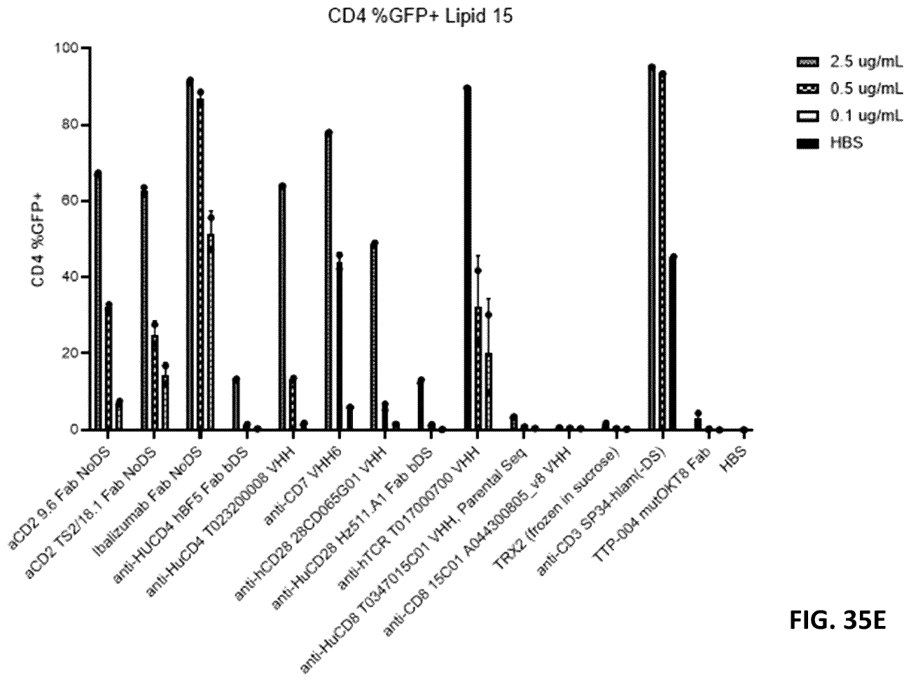

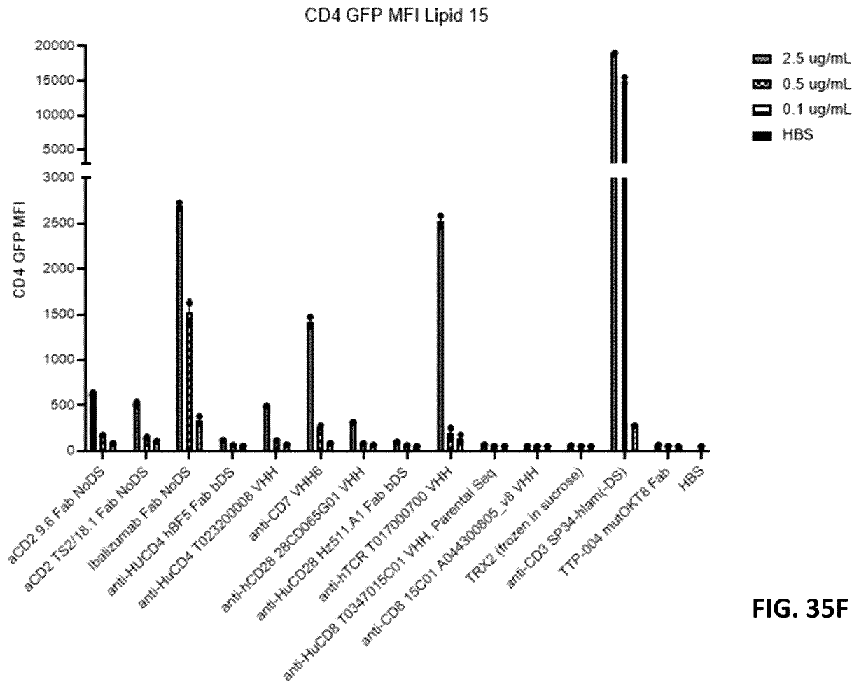

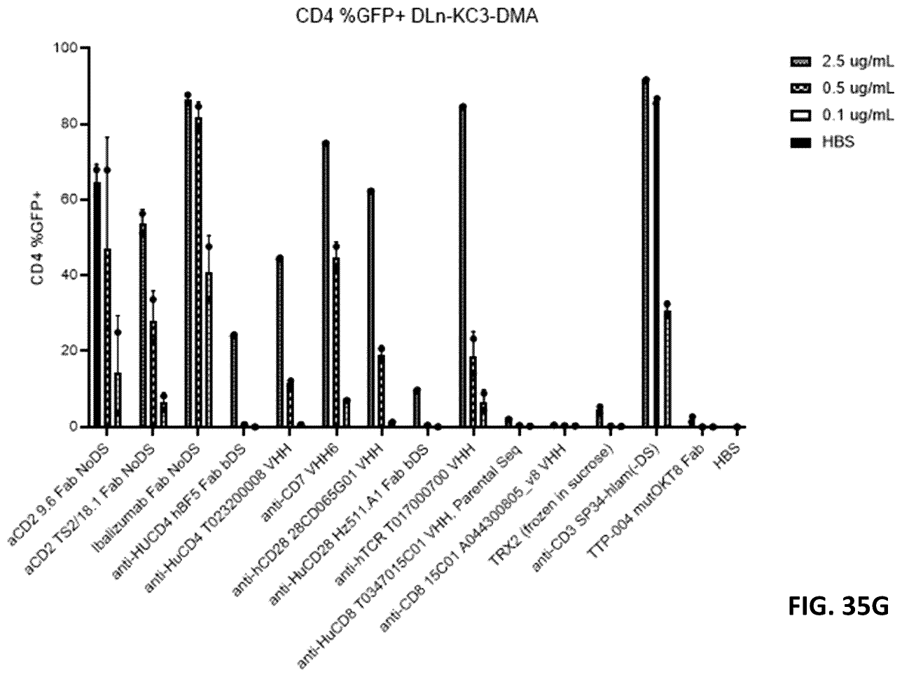

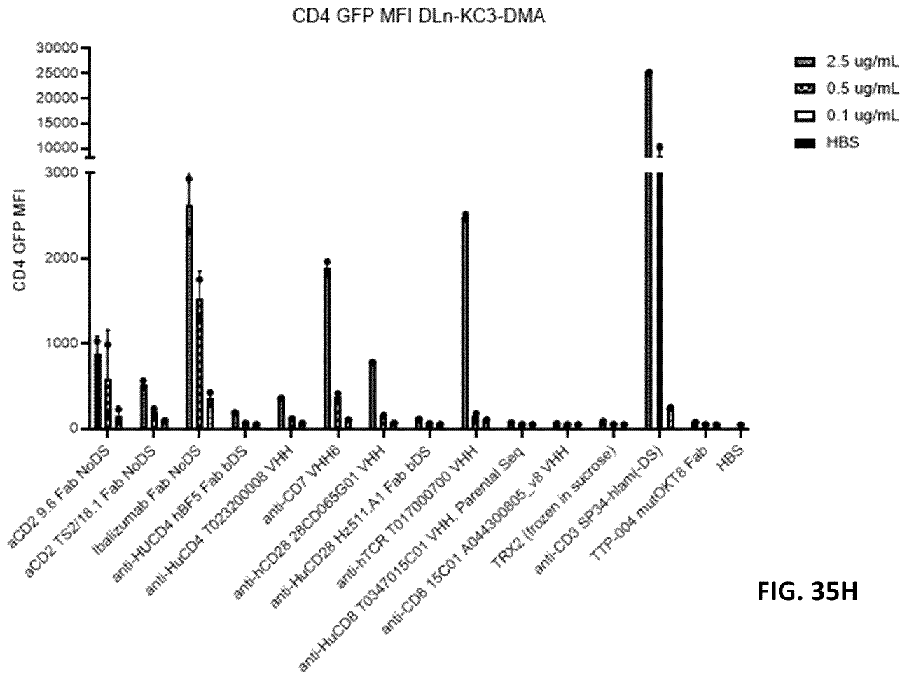

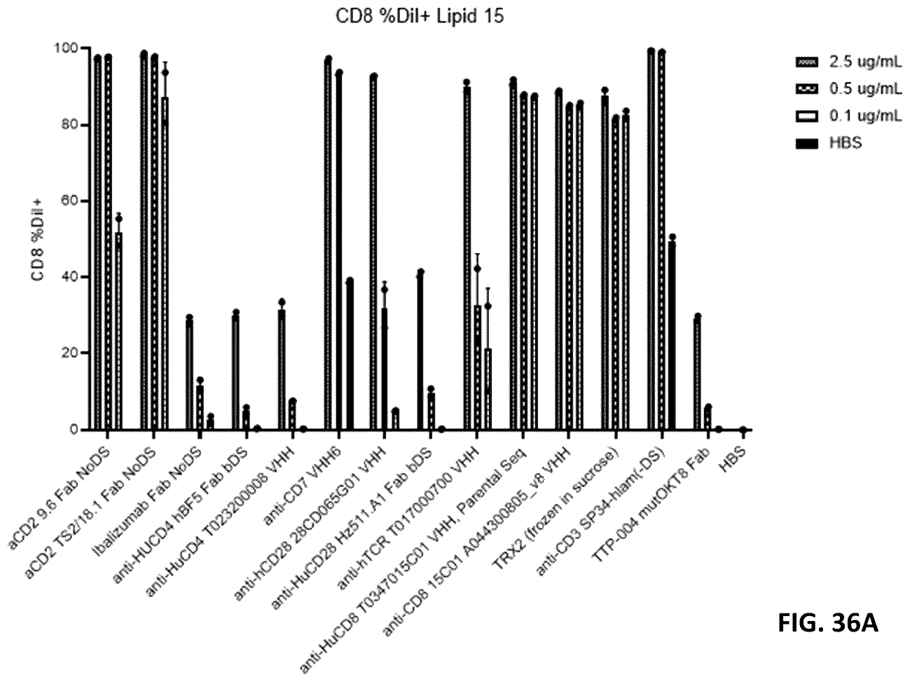

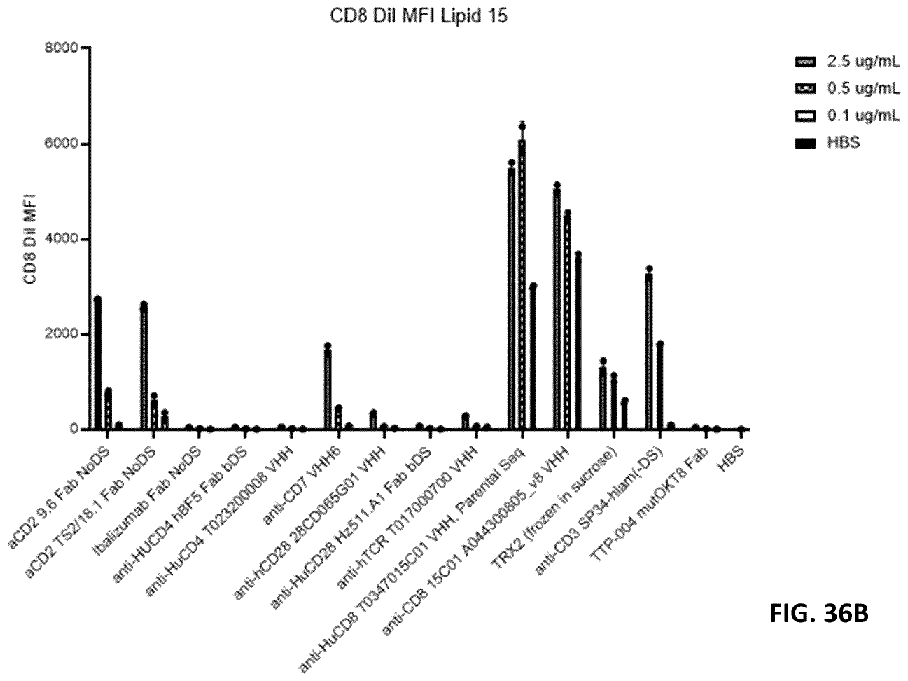

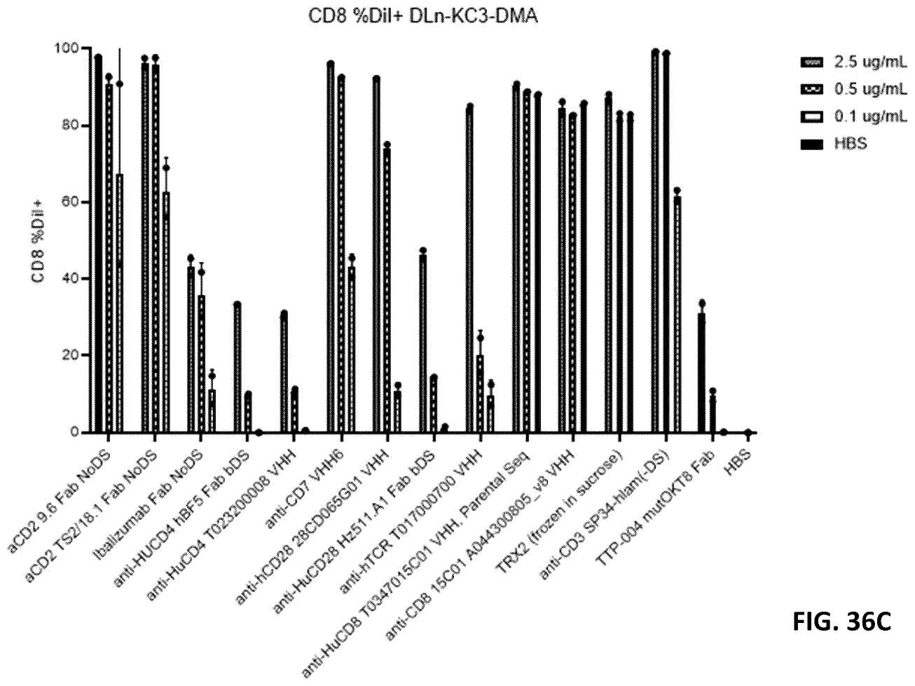

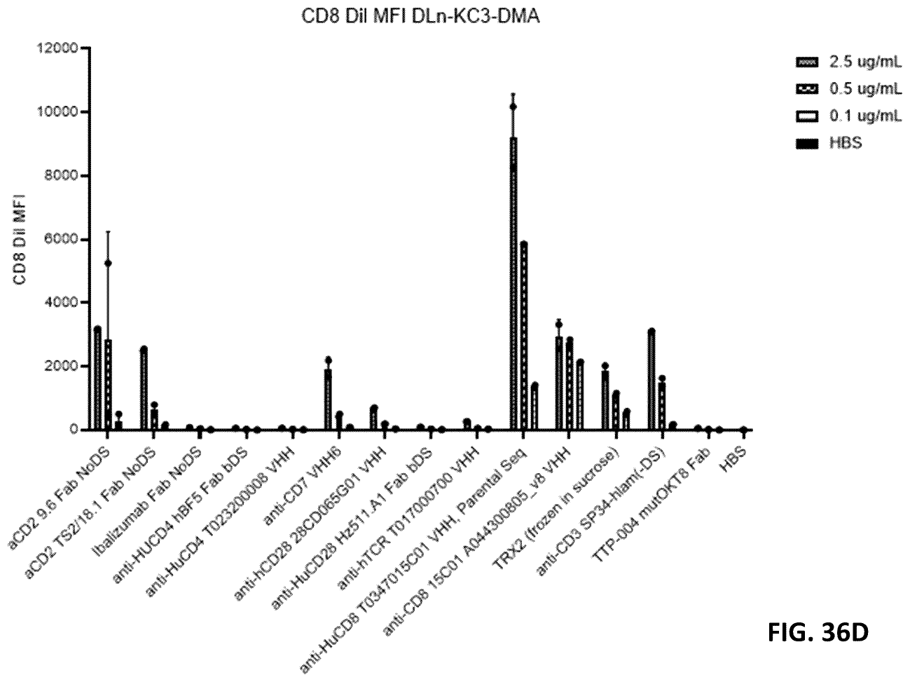

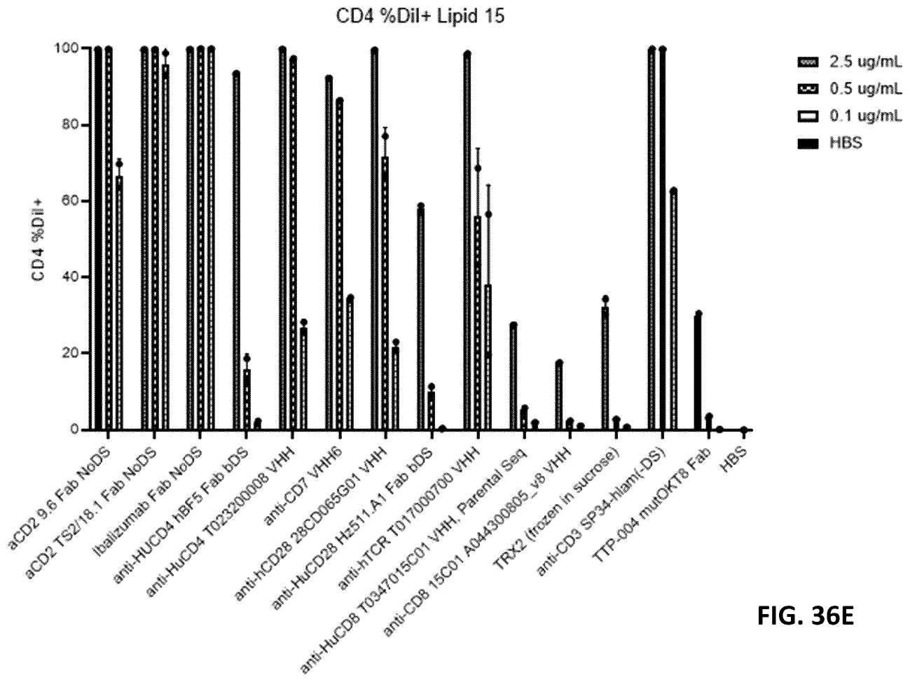

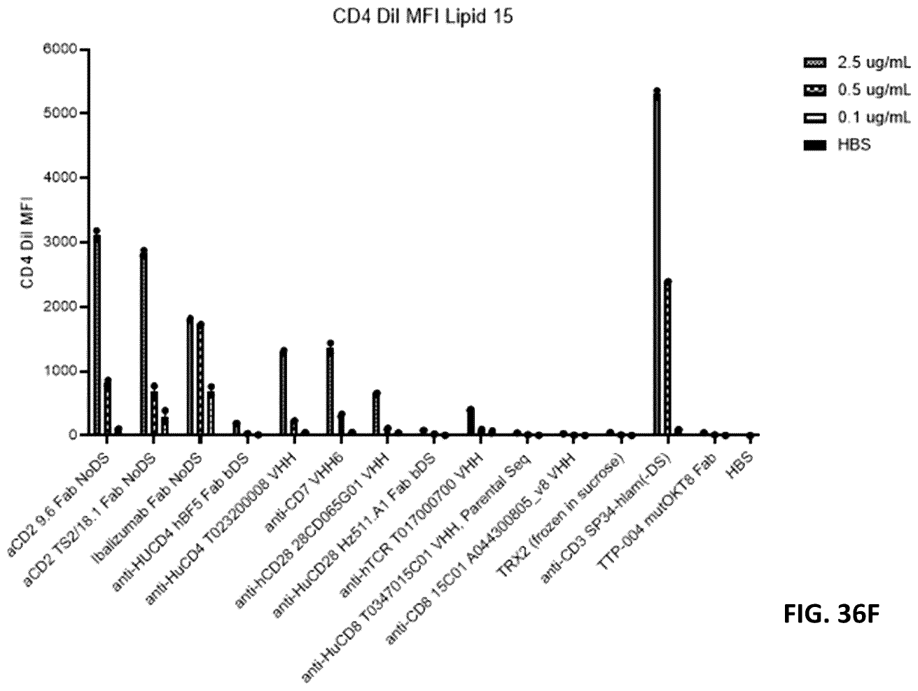

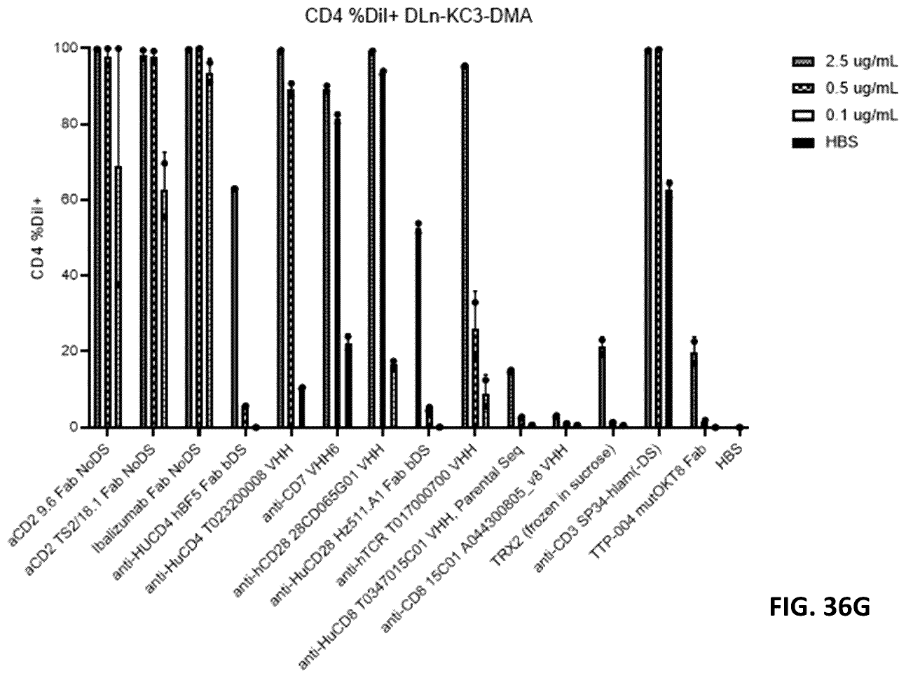

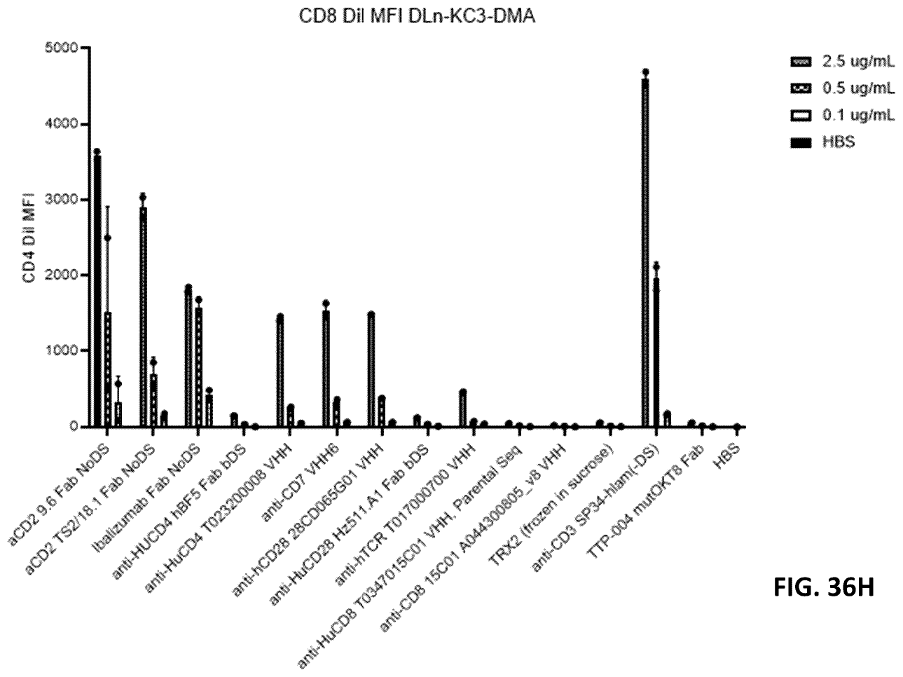

All of the clones evaluated mediated some level of transfection and GFP expression levels relative to the mutOKT8 LNP control for targeting both CD8 and CD4 T cell subset simultaneously with comparison of Lipid 15 (FIGS. 35A, 35B, 35E, 35F) and DLin-KC3-DMA LNPs (FIGS. 35C, 35D, 35G, and 35H). αCD3, αCD7, and TCR targeted LNPs resulted in GFP expression in both CD4 and CD8 T-cells while αCD8 and αCD4 targeted LNPs resulted in GFP expression selective in their corresponding subsets only as expected. Furthermore, non-binding (mutOKT8) control LNP resulted in no GFP expression in either cell type regardless of Lipid. At all dose level, Lipid 15 and DLin-KC3-DMA αCD2, αCD4, αCD7, αCD28, and TCR targeted LNPs showed similar levels of GFP expression indicating that the two lipids are equally efficient in this cellular uptake pathway not only with αCD3 and αCD8 targeted LNPs. Both αCD2 targeting Fabs showed lower levels of the CD8 and CD4 T-cell transfection population (both in terms of fraction of GFP+ T-cells as reflected by % GFP+ cells and the copies of GFP protein produced on a per cell basis as reflected by GFP MFI values) and LNP association measured as % DiI+(dye) T-cells and the DiI MFI reflective of the relative levels of DiI dye labeled LNPs taken up by the CD8 and CD4 T-cell population compared with αCD3 or αCD8 Fabs and nanobodies in both Lipid 15 and DLin-KC3-DMA LNPs (FIGS. 36A, 36B, 36C, 36D, 36E, 36F, 36G, and 36H). Amongst the CD4 targeted Fabs and nanobody, Ibalizumab mediated higher % transfection and GFP expression levels in CD4+ T cells however it was lower than αCD3 hSP34 Fabs in both Lipid 15 and DLin-LC3-DMA LNPs. For targeting both CD8 and CD4 T cell subset simultaneously αCD7 and anti-TCR clone show greater transfection and LNP association between both cell subsets at highest mRNA dose than that of the mutOKT8 post inserted LNPs with Lipid 15 and DLin-KC3-DMA. However both were lower than αCD3 hSP34 Fab. αCD28 targeting Fab and Nanobody show CD8 and CD4 T cell GFP transfection and expression levels only slightly greater than that of the mutOKT8 post inserted particles in both Lipid 15 and DLin-KC3-DMA LNPs.

This data indicates that across different targeting Fabs or Nanobodies like αCD2, αCD4, αCD7, αCD28 and TCR, both Lipid 15 and DLin-KC3-DMA LNPs are equally efficient and show very relative effect on transfection.

### Example 46. Physiochemical Properties of Lipids 10, 15, 16, 24A, and 26 and Comparator Lipid ALC-0315 LNPs

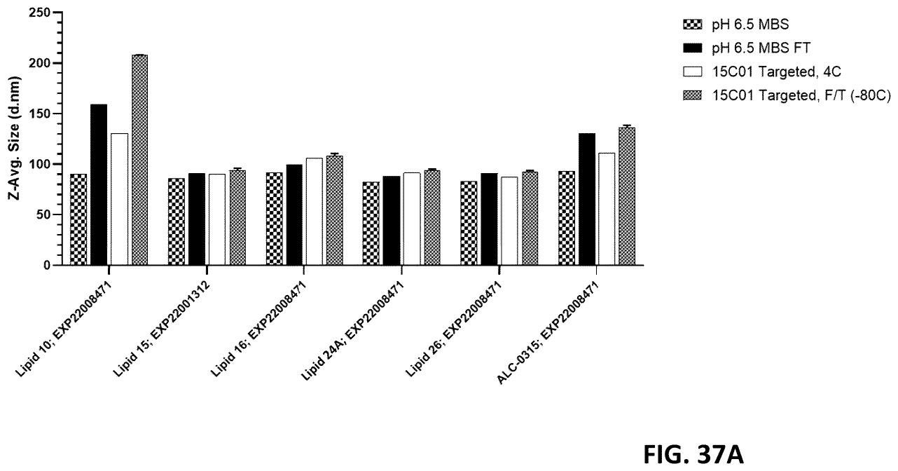

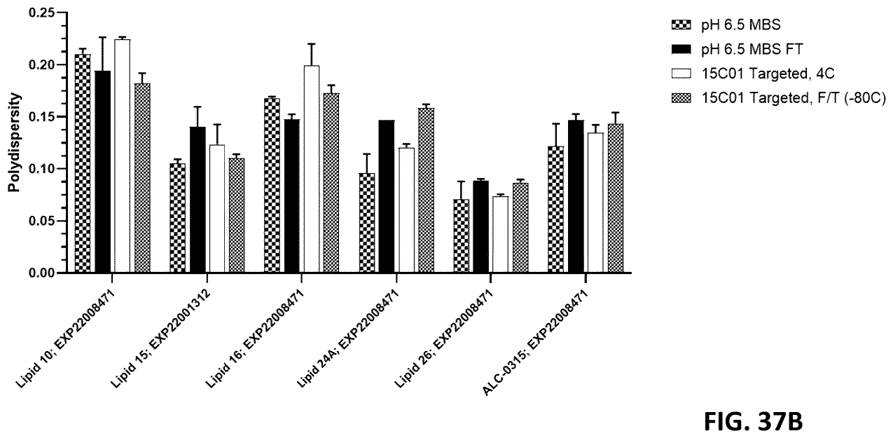

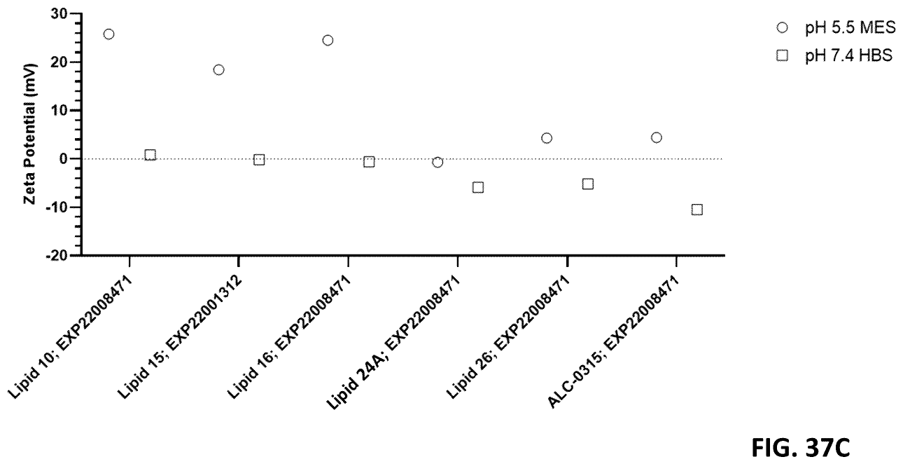

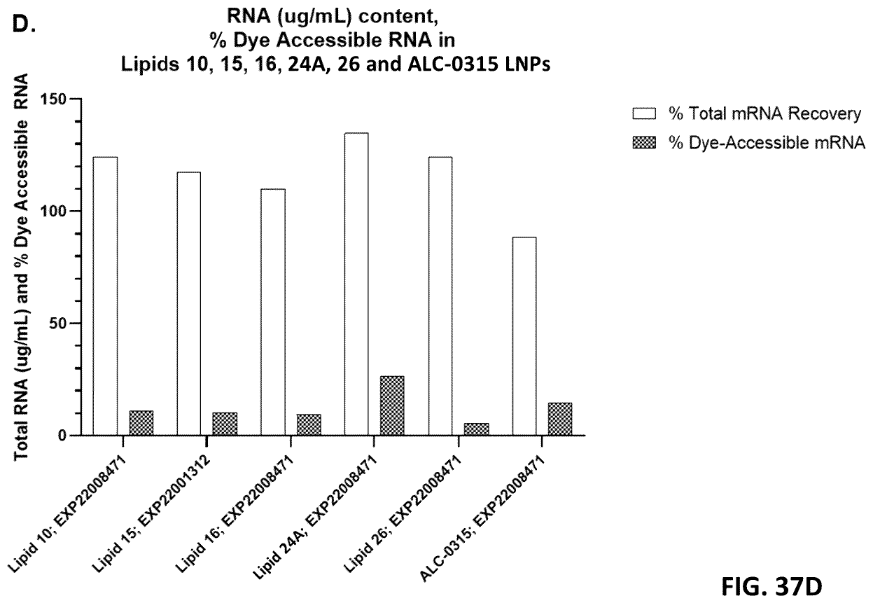

Lipids 10, 15, 16, 24A, 26, and ALC-0315 LNPs encapsulating GFP RNA (TriLink Biotechnologies Inc.) were prepared and characterized using methods described in Examples 5 to 8. Measured LNP size, PDI, charge and RNA content values of Lipids 10, 15, 16, 24A, 26, and ALC-0315 LNPs are summarized in Table 29, Table 30, Table 31, and FIGS. 37A to 37D. As seen in FIG. 37A, all lipids resulted in LNP sizes <100 nm in pH 6.5 MES buffer. Lipid 10 and ALC-0315 LNPs exhibited a notable size increase upon freeze-thaw (with 10% sucrose in MES pH 6.5 buffer), however, Lipids 15, 16, 24A, and 26 LNPs exhibited better Freeze-Thaw stability with minimal impact on particle diameter. Slight increase in LNP diameter was observed upon targeting antibody insertion (in pH 6.5 MES using a 37° C. incubation for 4 hours) for Lipids 15, 16, 24A, and 26, while larger increase in diameter was observed for Lipid 10 and ALC-0315 LNPs, and a similar trend of Freeze Thaw stability was observed for the targeted LNPs. All LNPs exhibited moderate to high encapsulation efficiency (<15% Dye accessible RNA) except for ALC-0315 LNPs where higher dye accessible RNA was observed. Lipid 10, 15 and 16 LNPs exhibited strongly positive charge in acidic pH (5.5) while near neutral charge in physiological pH (7.4). In contrast, Lipids 24A, 26 an ALC-0315 LNPs exhibited a relatively weak positive charge in acidic pH (5.5) and a slight negative charge at physiological pH (7.4) suggesting the role of lipid tail chemistry that results in such interfacial presentation of the ionizable head group that drives down the LNP apparent pKa. In summary, all lipids tested resulted in viable GFP mRNA encapsulation and freeze-thaw stability as well as <150 nm final targeted LNP diameters except Lipid 10 LNP that exhibited targeted LNP average diameter ˜ 200 nm post Freeze-Thaw. The ability of lipids 10, 15, 16, 24A, 26, and ALC-0315 LNPs for inducing in vitro GFP protein expression in primary human T-cells mediated by αCD8 T-cell receptor targeting was evaluated as described in Examples 12 and 13.

TABLE 29
Lipids 10, 15, 16, 24A, 26, and ALC-0315 LNPs Size, Polydispersity (DLS) data in pH 6.5 MBS and
post αCD8 targeting (T8) conjugate insertion

| | | Z-Avg. | | | Polydispersity | |
|---|---|---|---|---|---|---|
| | Z-Avg. | Size (nm); | Z-Avg. | Polydispersity | (DLS); | Polydispersity |
| Ionizable lipid, | Size (nm); | post-insertion; | Size (nm); | (DLS); | Post-insertion; | (DLS); |
| LNP no. | MBS | MBS | Post F/T | MBS | MBS | Post F/T |
| Lipid 10, DPG-PEG; | 90 | 131 | 208 | 0.21 | 0.22 | 0.18 |
| EXP22008471-3q | | | | | | |
| Lipid 15, DPG-PEG; | 86 | 90 | 94 | 0.11 | 0.12 | 0.11 |
| EXP22001312-3p | | | | | | |
| Lipid 16, DPG-PEG; | 92 | 106 | 109 | 0.17 | 0.20 | 0.17 |
| EXP22008471-4a | | | | | | |
| Lipid 24A, DPG-PEG; | 82 | 91 | 94 | 0.10 | 0.12 | 0.16 |
| EXP22008471-3t | | | | | | |
| Lipid 26, DPG-PEG; | 83 | 87 | 93 | 0.07 | 0.07 | 0.09 |
| EXP22008471-3u | | | | | | |
| ALC-0315, DPG- | 93 | 111 | 136 | 0.12 | 0.13 | 0.14 |
| PEG; EXP22008471- | | | | | | |
| ALC | | | | | | |

TABLE 30
Lipids 10, 15, 16, 24A, 26, and ALC-0315 LNPs Zeta
Potential (DLS) at pH 5.5 and pH 7.4

| | Charge | Charge |
|---|---|---|
| | (ZP, mV); | (ZP, mV); |
| Ionizable lipid, LNP no. | pH 5.5 | pH 7.4 |
| Lipid 10, DPG-PEG; EXP22008471-3q | 25.8 | 0.8 |
| Lipid 15, DPG-PEG; EXP22001312-3p | 18.4 | −0.2 |
| Lipid 16, DPG-PEG; EXP22008471-4a | 24.5 | −0.6 |
| Lipid 24A, DPG-PEG; EXP22008471-3t | −0.7 | −5.9 |
| Lipid 26, DPG-PEG; EXP22008471-3u | 4.3 | −5.2 |
| ALC-0315, DPG-PEG; EXP22008471-ALC | 4.4 | −10.5 |

TABLE 31
Lipids 10, 15, 16, 24A, 26 LNP Dye Accessible
RNA and total RNA content

| | Nominal | Measured | Dye- |
|---|---|---|---|
| | mRNA | Total | accessible |
| | Conc. | mRNA | mRNA |
| Ionizable lipid, LNP no. | (μg/mL) | (μg/mL) | (%) |
| Lipid 10, DPG-PEG; EXP22008471-3q | 150 | 124.30 | 10.9 |
| Lipid 15, DPG-PEG; EXP22001312-3p | 150 | 117.60 | 10.3 |
| Lipid 16, DPG-PEG; EXP22008471-4a | 150 | 109.90 | 9.40 |
| Lipid 24A, DPG-PEG; EXP22008471-3t | 150 | 134.90 | 26.4 |
| Lipid 26, DPG-PEG; EXP22008471-3u | 150 | 124.40 | ≤6 (5.5) |
| ALC-0315, DPG-PEG; | 150 | 88.50 | 14.70 |
| EXP22008471-ALC | | | |

### Example 47. In Vitro Protein Expression (GFP) in Primary Human T-Cells of Lipids 10, 15, 16, 24A, 26, and ALC-0315 aCD8 (T8) Targeted LNPs (Both Stored at 4° C. and Post Freeze-Thaw)

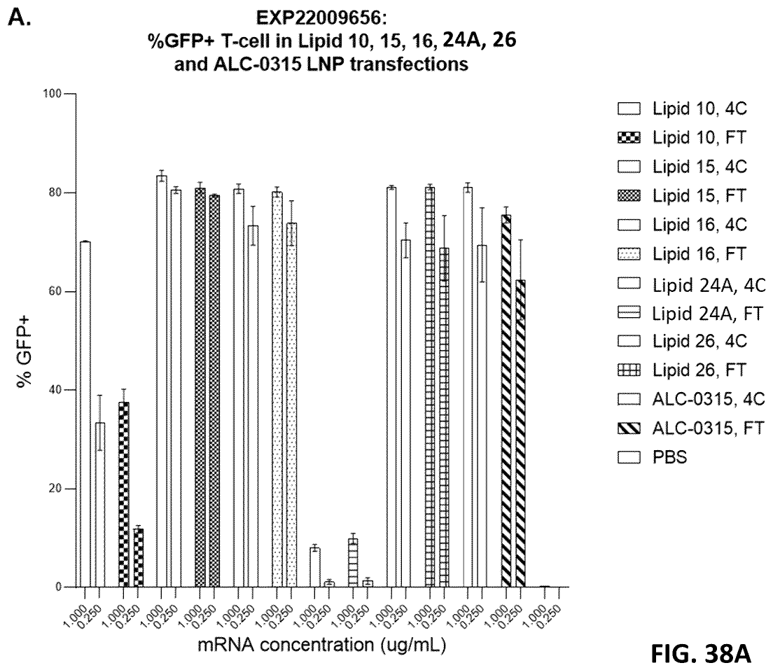

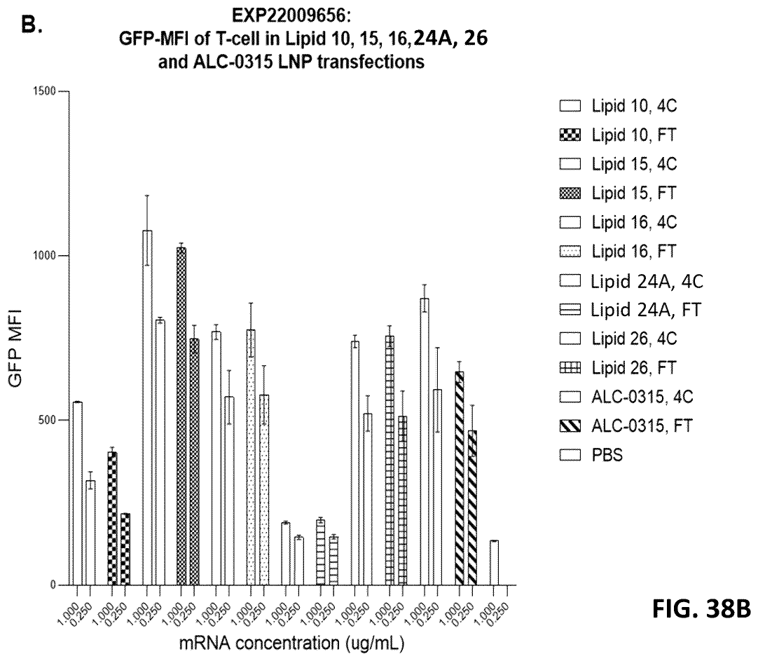

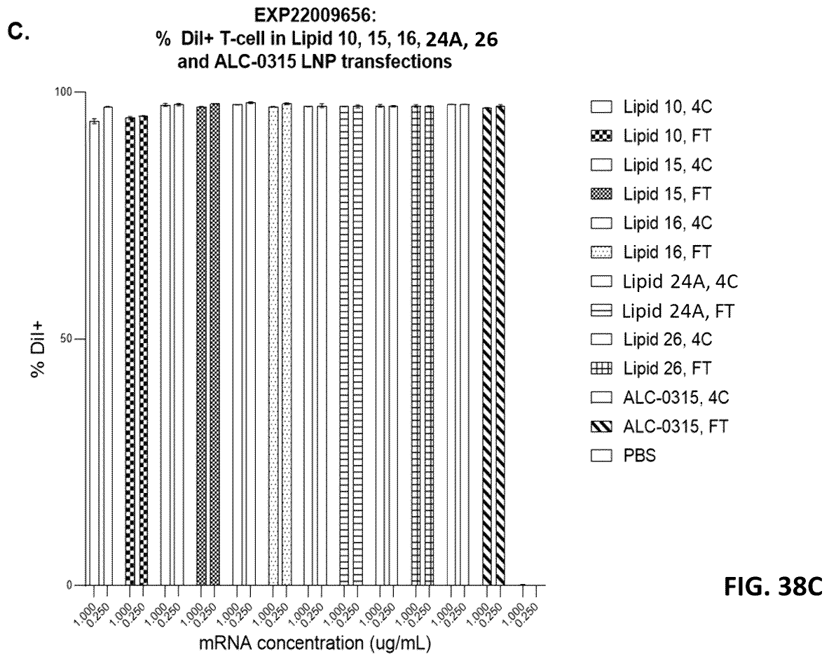

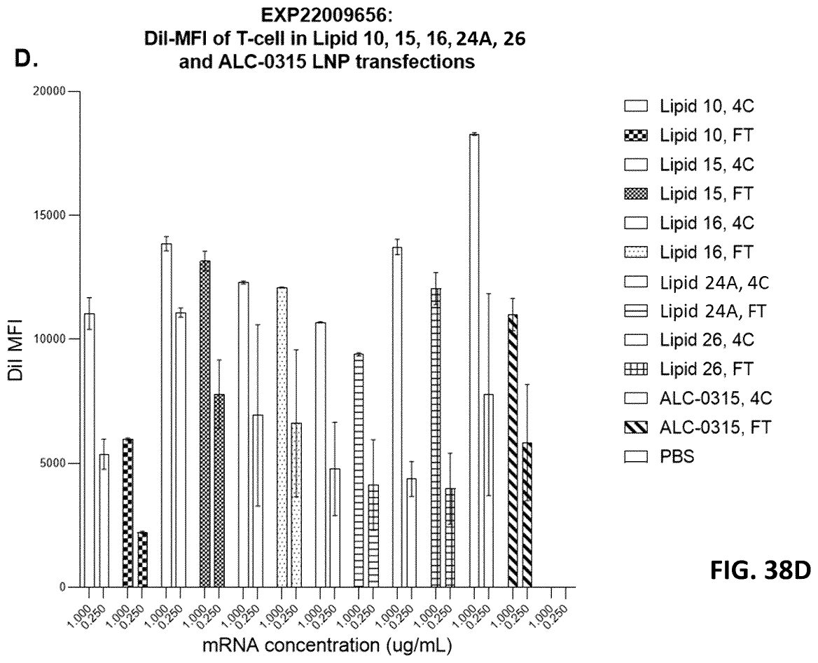

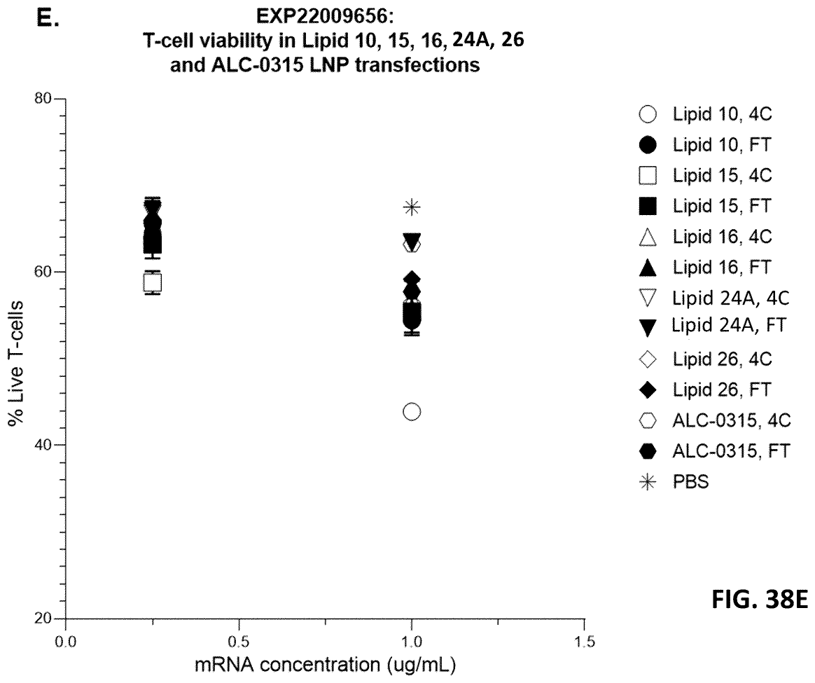

This example compares the GFP protein expression resulting from LNP's derived from Lipids 10, 15, 16, 24A, 26, and ALC-0315 (both before and after one Freeze-Thaw cycle). Nanoparticles bearing GFP encoding mRNA (and optionally a fluorescent dye label (DiI-C18-5DS)) were produced using the microfluidic mixing and buffer exchange processes described in Example 2. An αCD8 Fab-conjugate, T8, was incorporated into the parent LNPs to obtain the final Antibody targeted LNP formulation using the process described in Example 5. Particles thus produced were tested in vitro in primary human CD8 T cells to assess reported gene expression. As seen in FIG. 38C and FIG. 38D, with this T8 αCD8 targeting strategy, all LNPs tested showed similar high % DiI+(dye) T-cells and the DiI MFI reflective of equally efficient LNP association with CD8 T-cell population. However, notable differences in GFP protein expression levels (as reflected by % GFP+ cells and the copies of GFP protein produced on a per cell basis as reflected by GFP MFI values) were observed (FIG. 38A and FIG. 38B). Lipid 15 LNPs outperformed the other Lipids within the group, with Lipids 16, 26, and ALC-0315 enabling similar and relatively high levels of protein expression. It is noteworthy that Lipid 15 and Lipid 16 LNP Zeta Potential values at pH 5.5 and pH 7.4 suggest a large shift in charge and consequently a strong ability for fusion with endosomal membranes (and endosome disruption/endosomal escape) upon acidification of the endosomal compartment. However, Lipid 26 and ALC-0315 LNP Zeta Potential values suggest a relatively small charge shift under endosomal acidification. Hence, despite potentially a lower ability for charge driven endosomal membrane destabilization, as evidenced by the high protein expression levels, Lipid 26 and ALC-0315 LNPs enable potent cytosolic delivery of the GFP RNA payload. This suggests potential role of the branched Lipid tail group structure towards greater membrane fluidity and a more pronounced tail group “cone” shape, contributing towards greater endosomal membrane fusion, membrane destabilization and efficient endosomal escape of the RNA payload. Lipids 10, 15, 16, 24A, 26, and ALC-0315, αCD8 (T8) targeted LNPs were well tolerated by primary human T-cells (FIG. 38E).

### Example 48. Physiochemical Properties (Pre- and Post-Insertion) of DLin-KC3-DMA GFP and Bite Containing Lipid Nanoparticles (LNPs)

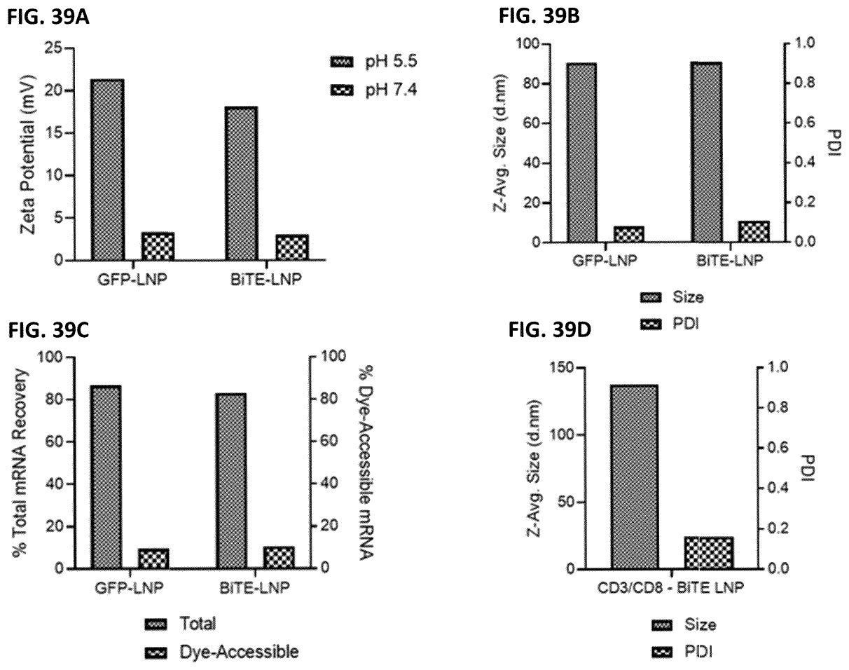

DLin-KC3-DMA LNPs encapsulating GFP-RNA (custom made by TriLink Biotechnologies Inc.) or BiTE mRNA (custom made by Vernal Biosciences) were prepared using methods described in Example 6 and characterized using methods described in Example 8. Measured LNP size and PDI, zeta potential, and mRNA recovery of DLin-KC3-DMA LNPs pre-insertion and LNP and PDI post-insertion are summarized in Table 32 and in FIGS. 39A to 39D. As seen in FIG. 39B, preparation of GFP and BiTE LNPs with DLin-KC3-DMA resulted in LNP sizes <100 nm. Similarly, polydispersity of both LNPs remained <0.2. Both LNPs exhibited moderate to high encapsulation efficiency (<15% Dye accessible RNA) and >80% RNA recovery (FIG. 39C). As seen in FIG. 39D, anti-CD3 and anti-CD8 insertion into BiTE LNPs with DLin-KC3-DMA resulted in LNP sizes <140 nm and polydispersity ≤0.2.

TABLE 32
DLin-KC3-DMA LNP size and PDI, zeta potential, and mRNA recovery pre-insertion and
size and PDI post-insertion.

| | Z-Avg. | | Charge | Charge | mRNA | Dye |
|---|---|---|---|---|---|---|
| Ionizable Lipid, | Size | PDI | (ZP, mV); | (ZP, mV); | Recovery | accessible |
| LNP no. | (nm); | (DLS); | pH 5.5 | pH 7.4 | (%); | (%); |
| DLin-KC3- | 91.53 | 0.08 | 21.4 | 3.32 | 86.9 | 9.4 |
| DMA, GFP; | | | | | | |
| DLin-KC3- | 91.33 | 0.10 | 18.1 | 2.97 | 83.3 | 10.6 |
| DMA, BiTE; | | | | | | |
| CD3/CD8, DLin- | 137.6 | 0.22 | — | — | — | — |
| KC3-DMA, | | | | | | |
| BiTE; | | | | | | |

### Example 49. Viability, LNP Association (Measured as DiI-Dye Fluorescence), and GFP Expression in Murine T-Cells Transfected with aCD3 (500A2), aCD4 (GK1.5), and aCD8 (YTS156.7.7) Targeted DLin-KC3-DMA LNPs

Particles produced were tested in vitro in primary murine CD3+ T cells to assess T-cell viability, LNP association (DiI fluorescence), and GFP protein expression. Following euthanasia by cervical dislocation spleens of 6-8-week-old female Balb/c mice were harvested, cut into small fragments, and passed through a 70 μm cell strainer. Following wash in cold PBS, splenocytes were counted and CD3+T lymphocytes were purified using direct negative magnetic isolation following the manufacturer's instructions (StemCell Technologies, Vancouver, Canada). Isolated CD3+ murine T-cells were plated in six-well plates at a concentration of 1×106 cells/mL in RPMI-1640, supplemented with 10% FBS, 1×ITS, 55 μM 2-Mercaptoethanol, and 20 ng/mL recombinant murine IL-2, and 5 ng/mL recombinant murine IL-7. Cells were rested for at least 2 hours at 37° C. preceding treatment.

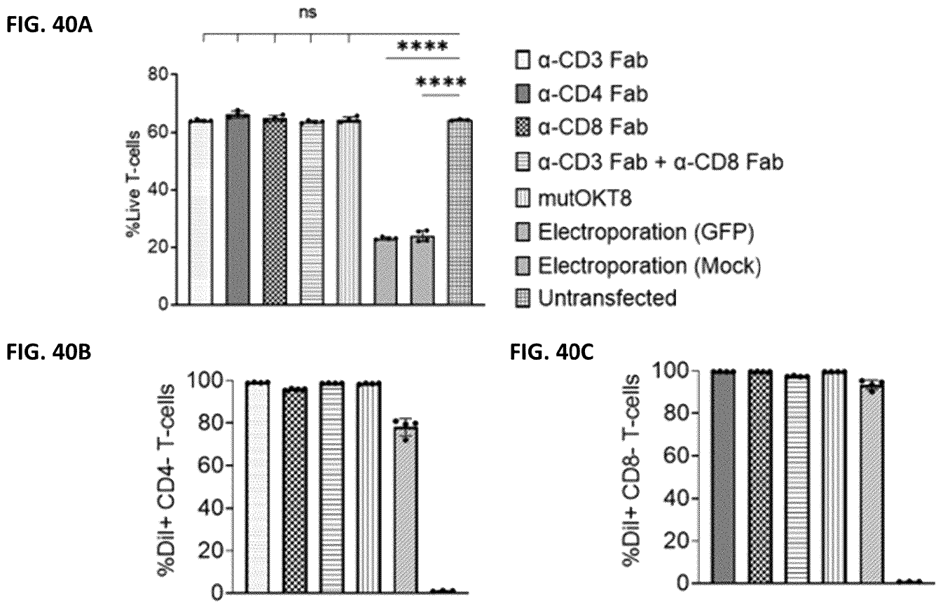

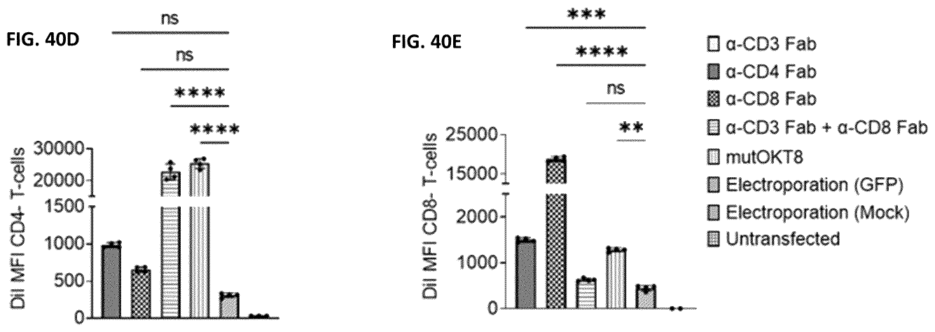

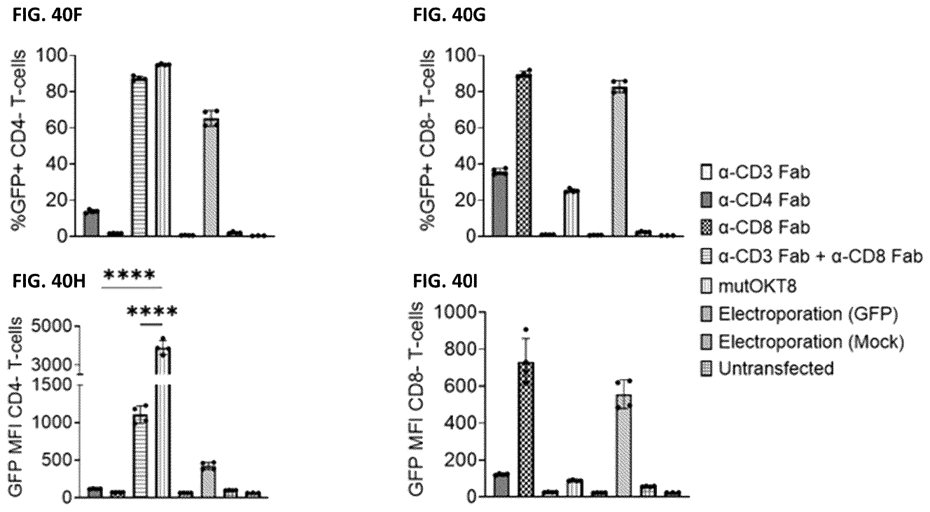

All targeted lipid formulations with GFP payloads were well tolerated by primary murine T-cells at the 1 ug/mL per 100,000 T-cells dose level (FIG. 40A). The association of LNPs to specific T-cell subsets were assessed by evaluating the signal of the incorporated DiI dye. 24 hours post-transfection, an increased DiI mean fluorescence intensity (MFI) signal was observed in the T-cell subset corresponding to the specific targeting moiety (FIG. 40B, FIG. 40C, FIG. 40D. FIG. 40E). To evaluate mRNA transfection the ability of the targeted LNPs to deliver mRNA encoding the reporter protein enhanced green fluorescent protein (GFP) to murine primary T-cells was assessed. Analogous to the particle association data, increased GFP expression was observed in the targeted T-cell subsets over the subsets negative for the specific targeting moiety (FIG. 40F, FIG. 40G, FIG. 40H. FIG. 40I). Additionally, to investigate whether CD3 and CD8 co-targeting could work in synergy and thereby increase the mRNA delivery efficiency over CD3 and CD8 single targeting, murine T-cells were treated with LNPs post-inserted with both anti-CD3 and anti-CD8 Fabs. Co-targeting proved to increase GFP expression in CD4− T-cells compared to CD3 single targeting and CD8 single targeting, respectively (FIG. 40H).

### Example 50. Transfection of Murine T-Cells with Different aCD3, aTCR, aCD4, and aCD8 Targeting Densities in DLin-KC3-DMA LNPs

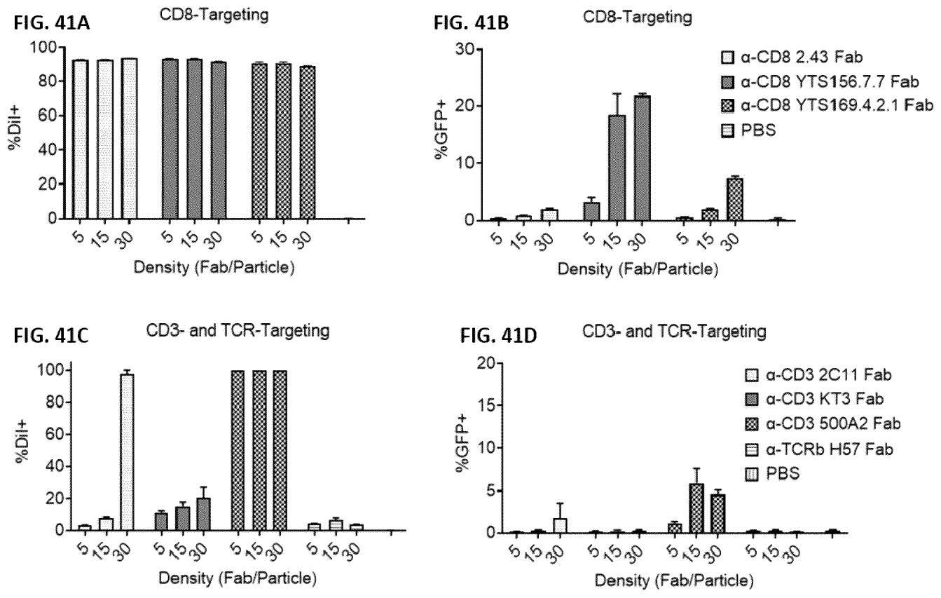

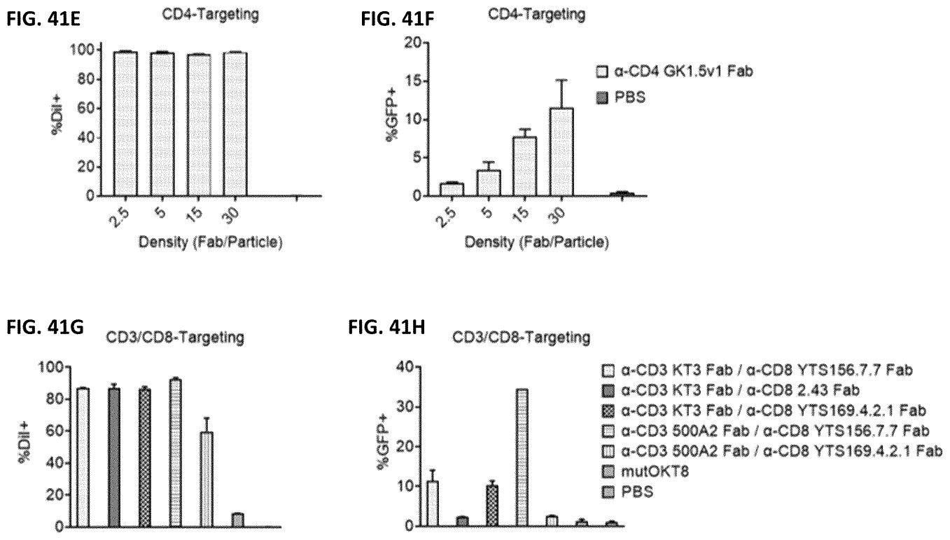

This example compares transfection of murine T-cells with different αCD3, αTCR, αCD4, and αCD8 targeting densities inserted into DLin-KC3-DMA LNPs (FIGS. 41A to 41H). Particles produced were tested in vitro in primary murine CD3+ T cells to assess LNP association (DiI) and reporter protein expression (GFP). Density of 30 Fabs/LNP was observed to give increased transfection efficiency (GFP) for CD8- and CD4-targeting moieties (FIG. 41B and FIG. 41F). Density of 15 Fabs per LNP (Fab/Particle) was observed to give increased transfection efficiency (GFP) for CD3- and TCR-targeting moieties (FIG. 41D).

### Example 51. Activation, Cytokine Release, Phenotyping, and Gene Expression Analysis in Murine T-Cells Transfected with aCD3 (500A2) and aCD8 (YTS156.7.7) Targeted DLin-KC3-DMA LNPs

This example compares the activation, cytokine release, phenotyping, and gene expression profiles in murine T-cells transfected with CD3-targeted DLin-KC3-DMA LNPs to those transfected with CD8-targeted DLin-KC3-DMA LNPs. Gene expression profiles of LNP-transfected CD8+ T-cells were assessed using nCounter Mouse PanCancer Immune Profiling Panel (NanoString Technologies, Washington, US), characterizing 770 murine immunology- and cancer-related genes. Briefly, CD8+ murine T-cells were isolated and transfected as described above. Cells were lysed using Buffer RLT (Qiagen, Hilden, Germany) and cell lysates were treated with Proteinase K (Thermo Fisher, Massachusetts, US) prior to hybridization following the manufacturer's instructions.

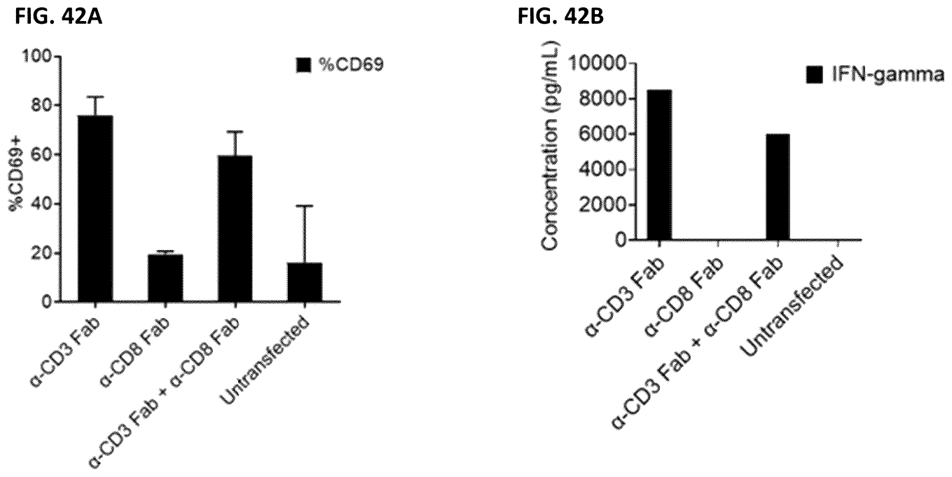

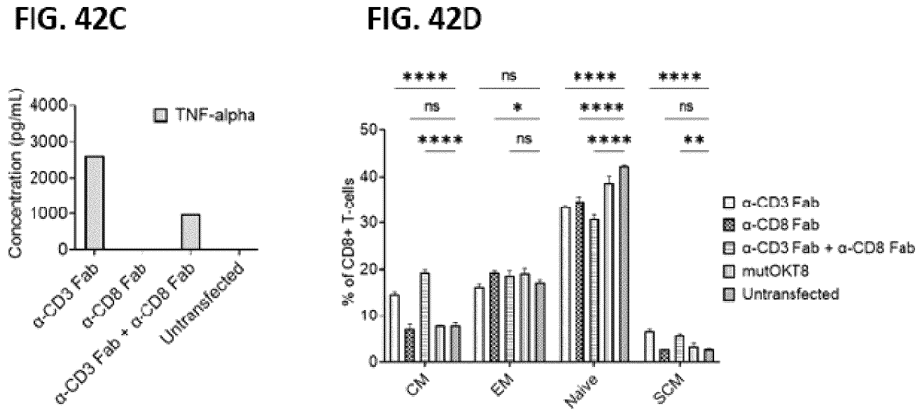

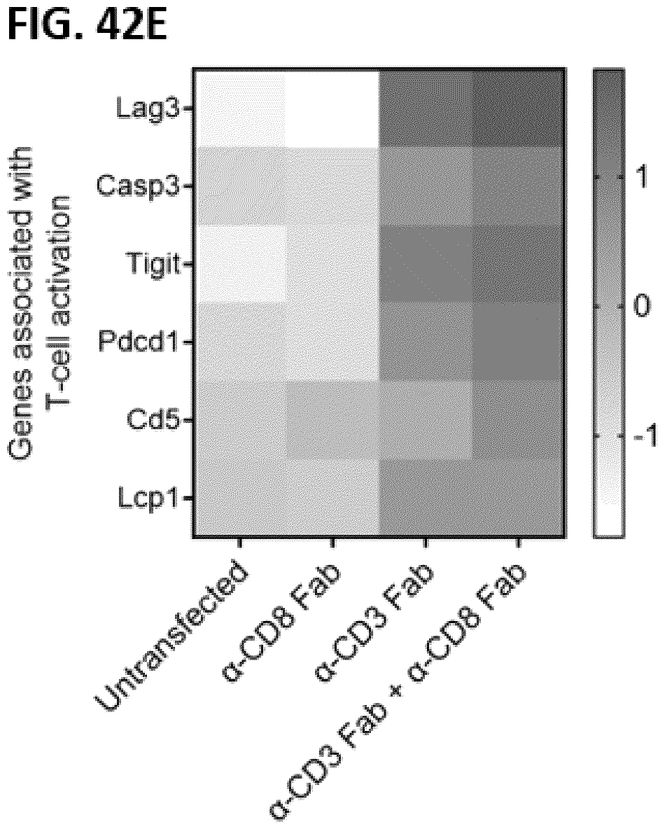

An upregulation of the early activation marker, CD69, was observed by flow cytometry in the CD3- and CD3/CD8-targeted groups but not in the CD8-targeted group (FIG. 42A). Additionally, increased levels of IFN-gamma and TNF-alpha was observed in the supernatants of T-cells treated with CD3-targeting LNPs (FIG. 42B and FIG. 42C). When evaluating the T-cell phenotypes 48 hours after LNP treatment, a shift from naïve toward memory subsets in groups treated with CD3-targeting LNPs was observed (FIG. 42D). The gene expression profiles of the treated T-cells were evaluated using the nCounter Mouse PanCancer Immune Profiling Panel from NanoString Technologies. Supporting the CD69 activation data, an upregulation of genes associated with T-cell activation was observed in the groups treated with CD3- or CD3/CD8-targeting LNPs compared to the group with CD8-targeting LNPs and the untreated group (FIG. 42E).

### Example 52. In Vivo T-Cell Reprogramming Using Mcherry Reporter Protein and DiI-Dye Fluorescence with aCD3 (500A2), aCD4 (GK1.5), and aCD8 (YTS156.7.7) Targeted DLin-KC3-DMA LNPs in Syngeneic Balb/C Mice

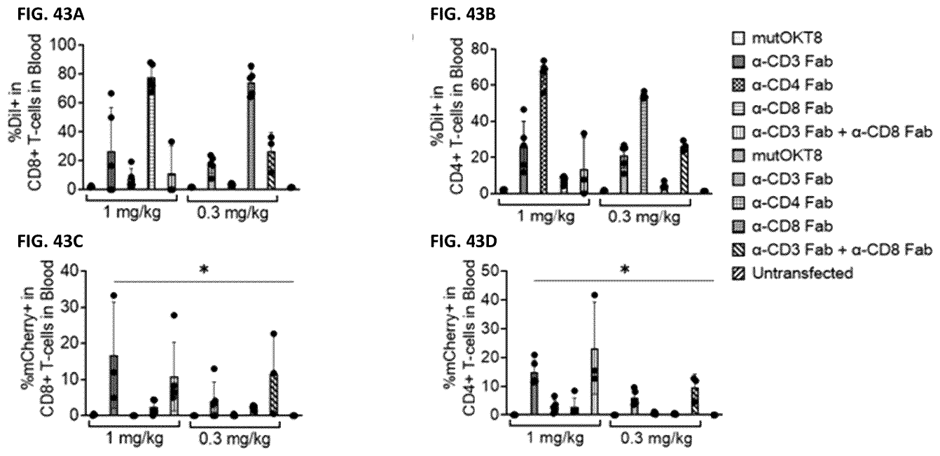

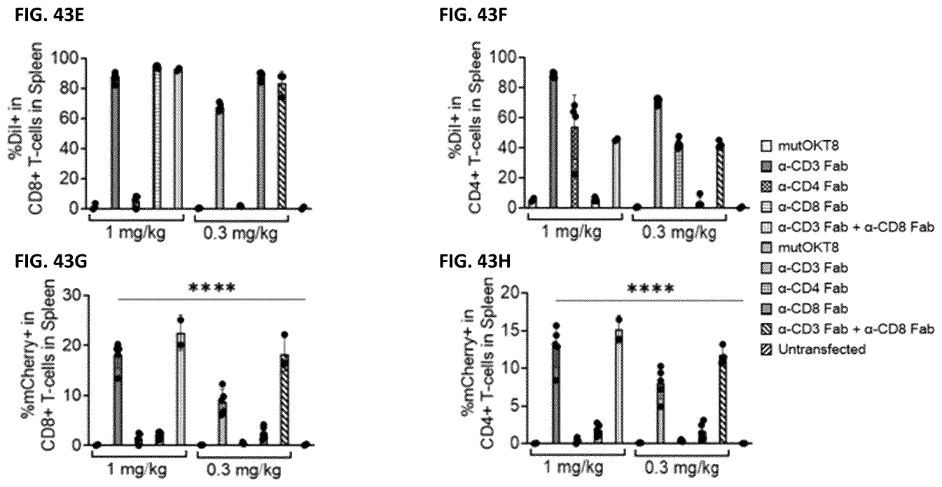

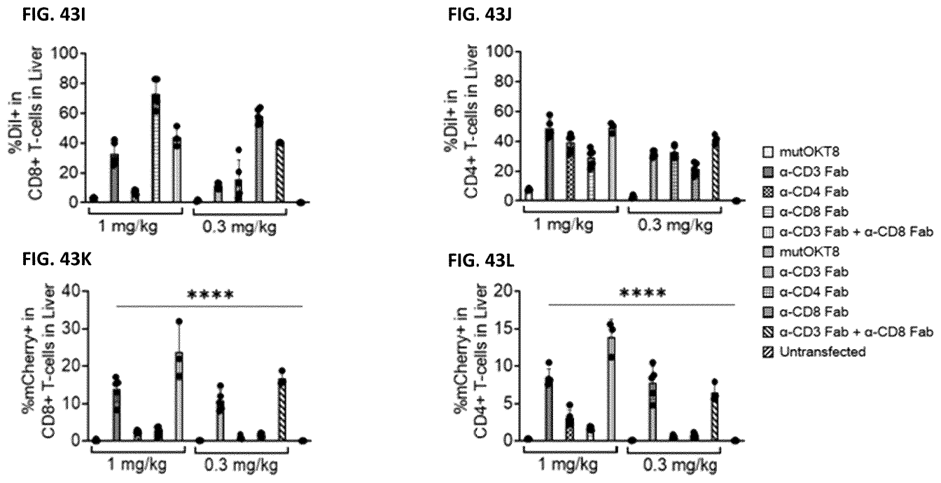

This example compares LNP association (DiI) and mCherry expression in wild type Balb/c mice treated with αCD3 (500A2), αCD4 (GK1.5), AND αCD8 (YTS156.7.7) targeted DLin-KC3-DMA LNPs to a non-targeted DLin-KC3-DMA LNP comparator. LNPs were intravenously injected into wildtype BALB/c mice through the tail vein at 0.3 mg/kg and 1 mg/kg. Mice were euthanized 24 hours after injection and selected tissues (blood, spleen, and liver) were harvested, processed, and stained for analysis by Flow Cytometry. ˜₇₈% of CD3+CD8+ T-cells in the blood showed LNP-association when targeted with anti-CD8 in comparison to untargeted LNPs showing <2% in the same cell population (FIG. 43A). Similarly, ˜67% of the CD3+CD4+ cell population were DiI-positive in mice treated with anti-CD4-targeted LNPs in contrast to ˜2% in the group treated with an equal dose of untargeted LNPs (FIG. 43B). In the spleen, a specific target-dependent cell association trend was observed for all groups treated with targeted LNPs over non-targeted LNPs (FIG. 43E and FIG. 43F). In contrast, T-cell subsets in the liver showed higher levels of non-specific LNP association (FIG. 43I and FIG. 43J).

The mRNA transfection efficiency was evaluated by assessing the levels of mCherry protein expression encoded in the encapsulated mRNA. mCherry expression was observed in groups treated with anti-CD3- and anti-CD3/CD8-targeted LNPs in all tissues analyzed (FIG. 43C, FIG. 43D, FIG. 43G, FIG. 43H, FIG. 43K, and FIG. 43L).

### Example 53. In Vitro Cytotoxicity Using Bite mRNA and aCD3/aCD8 Targeted LNPs Based on DLin-KC3-DMA Lipid

The IncuCyte NucLight Red Lentivirus reagent (Sartorius, Gottingen, Germany) was used to transduce CT26 cells following the manufacturer's protocol. Transduced NucLight Red positive cells were selected for using puromycin and >95% purity was confirmed by flow cytometry prior to co-culture assays. Murine CD3+ T-cells were transfected with DLin-KC3-DMAαCD3-, αCD4−, αCD8-, or αCD3/αCD8-targeted BiTE mRNA LNPs, DLin-KC3-DMAαCD3-, αCD4−, αCD8-, or αCD3/αCD8-targeted non-BiTE mRNA LNPs as a control, or recombinant BiTE protein. Unbound LNPs were removed by washing in PBS 4 hours post-transfection. Transfected T-cells were then co-cultured with NucLight Red Lentivirus transduced CT26 cells at varying effector-to-target cell ratios in T-cell media (RPMI-1640, 10% FBS, 1×ITS, 55 μM 2-Mercaptoethanol, 20 ng/mL recombinant murine IL-2, and 5 ng/mL recombinant murine TL-7) in a 96-well flat-bottom plate. Cancer cell killing was monitored in the SX5 IncuCyte every 3 hours. The level of cytotoxicity was quantified by normalizing the count of red cells to the initial time point.

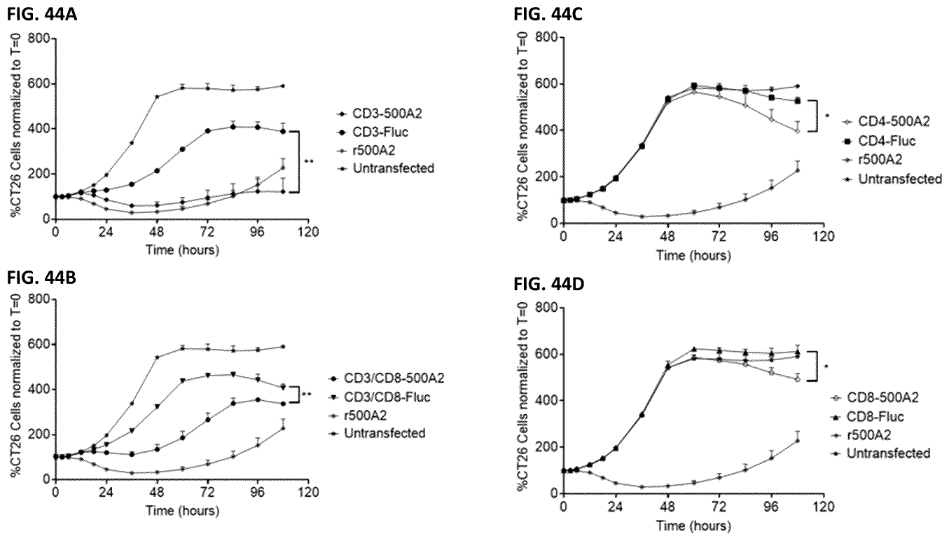

As seen in FIG. 44A, FIG. 44B, FIG. 44C, and FIG. 44D, αCD3-, αCD4−, αCD8-, or αCD3/αCD8-targeted BiTE mRNA LNPs resulted in a statistically significant increased cytotoxicity in comparison to the αCD3-, αCD4-, αCD8-, or αCD3/αCD8-targeted non-BiTE mRNA LNP controls.

### Example 54. In Vivo Efficacy Using BiTE mRNA and aCD3/aCD8 Targeted LNPs Based on DLin-KC3-DMA Lipid

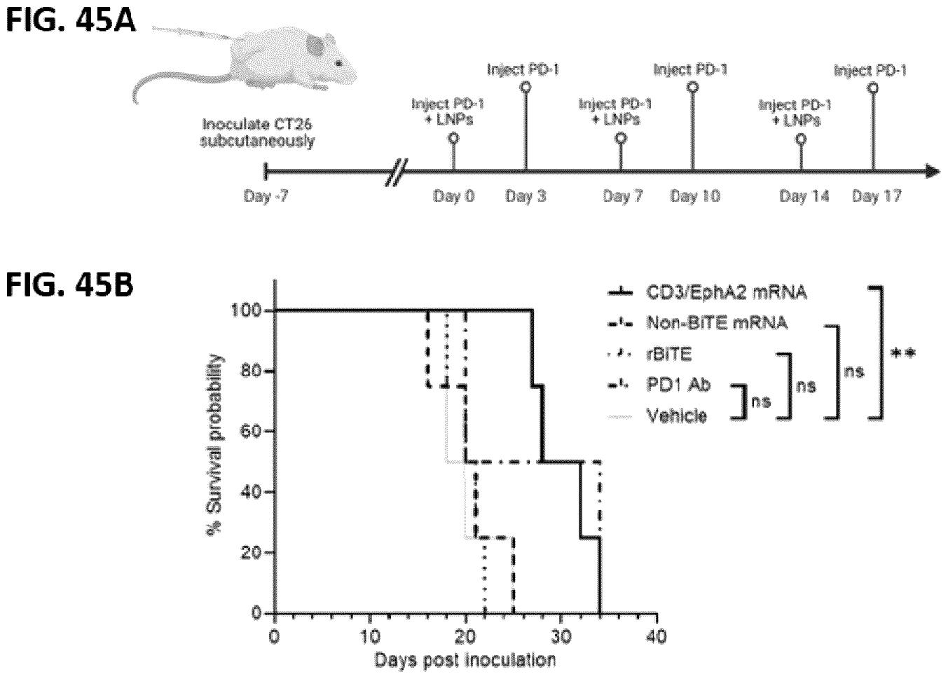

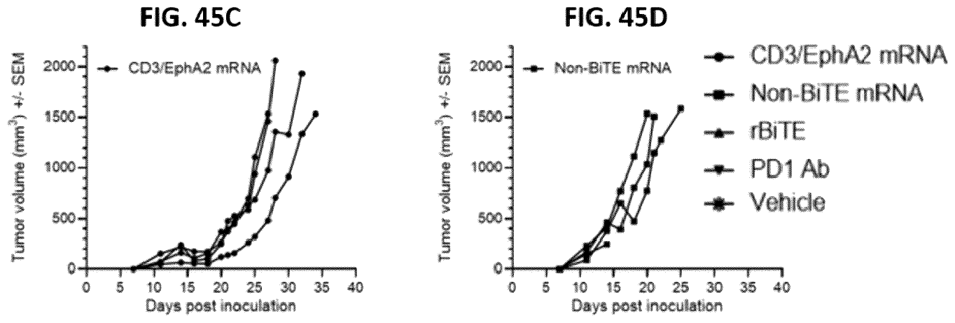

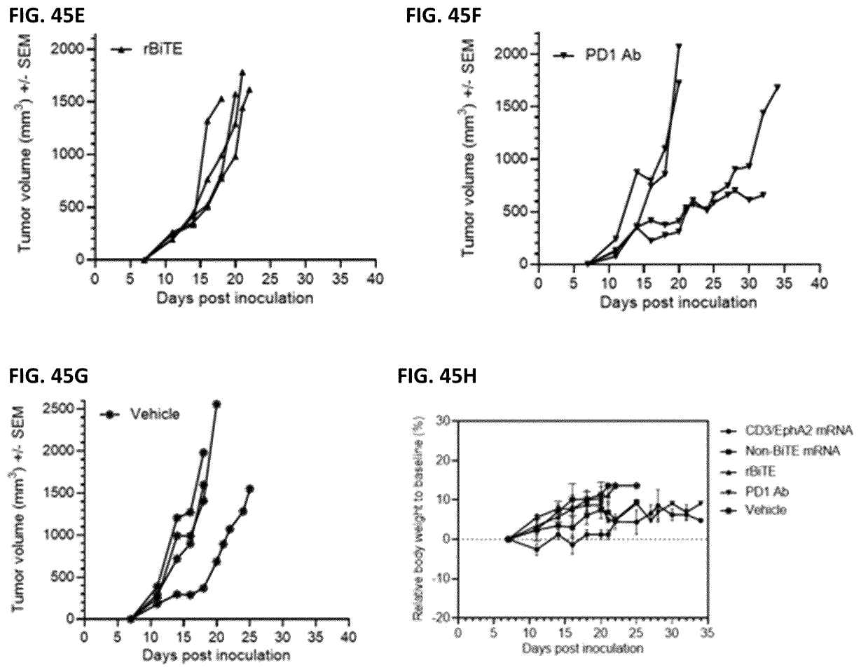

6-8-week-old female Balb/c mice (n=4 per group) were subcutaneously inoculated with 2.5×10⁵ CT26 cells into the right flank 7 days prior to treatment initiation (FIG. 45A). Mice were randomized and grouped based on tumor size. Anti-mouse PD-1 (clone RMP1-14) was administered intraperitoneally twice a week for a total of six doses at 10 mg/kg. DLin-KC3-DMAαCD3/αCD8 targeted BiTE mRNA LNPs, a non-BiTE mRNA LNP as a negative control, and recombinant anti-EphA2×CD3 BiTE were administered by intravenous injection into the tail vein once a week for a total of three doses at 0.2 mg/kg. Bodyweights and tumor sizes were monitored three times a week throughout the study period.

As seen in FIG. 45B, FIG. 45C, FIG. 45D, FIG. 45E, FIG. 45F, FIG. 45G, and FIG. 45H αCD3/αCD8 targeted BiTE mRNA LNPs resulted in reduced tumor burden and increased survival in comparison to the targeted non-BiTE mRNA LNP control. Furthermore, no statistically significant difference in survival was observed between the recombinant BiTE-treated group and the vehicle control at the tested dose.

### Details on BiTEs and Targeting Moieties Used:

mRNA was produced by Vernal Biosciences (Vermont, US). Briefly, GFP-, Fluc- and anti-EphA2×CD3 bi-specific T-cell engager (BiTE)-encoding mRNA were in vitro-transcribed, poly-A tailed, and capped (Cap1). Amino acid sequences for anti-mouse CD3 and EphA2 scFvs were derived from public sources (500A2 Genbank AAB81028.1, AAB81027.1; KT3 Genbank AVW80143.1; 2C11 EF063578.1; EphA2 UniprotP29317). BiTE mRNAs were designed with a mouse kappa chain-derived signal peptide, a 3×(G4S) linker between the VH and VL domain of each binder, a 4×(G4S) linker be-tween the two binders, and a FLAG-tag (Sequence: DYKDDDDK) at the 5′ end of the binding region.

Variable heavy and light chain amino acid sequences for anti-mouse CD3, TCR, CD8 and CD4 clones were derived from public sources (500A2 Genbank AAB81028.1, AAB81027.1; KT3 Genbank AVW80143.1; 2C11 EF063578.1; H57 PDB 1NFD, YTS105.18.10 PDB 2ARJ; YTS169.4.2.1, YTS156.7.7 and 2.43 AB030195; GK1.5 Genbank AAA51349.1, PMID 16901500). Fabs were produced in HEK, purified by IMAC, and formulated into PBS by Biointron (Taizhou, China).

### Example 55. Physiochemical Properties (Pre- and Post-Insertion) of Lipid 15 mCherry and CAR Containing Lipid Nanoparticles (LNPs)

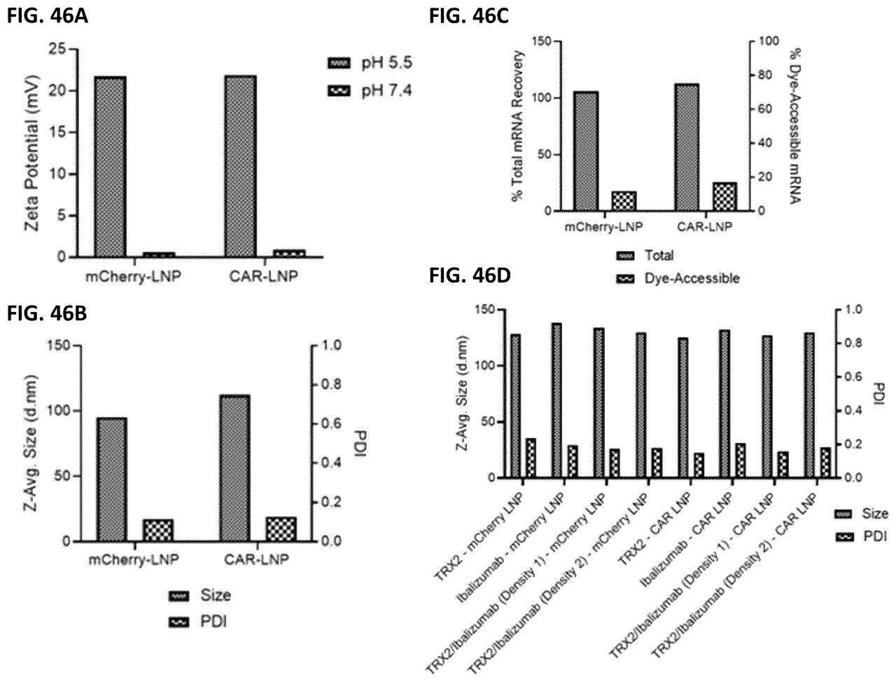

Lipid 15 LNPs encapsulating mCherry-RNA (custom made by TriLink Biotechnologies Inc.) or CAR mRNA (custom made by Vernal Biosciences) were prepared using methods described in Example 5 and characterized using methods described in Example 8. Measured LNP size and PDI, zeta potential, and mRNA recovery of Lipid 15 LNPs pre-insertion are summarized in Table 33 and in FIGS. 46A to 46D. As seen in FIG. 46B, preparation of mCherry and CAR LNPs with Lipid 15 resulted in LNP sizes <120 nm. Similarly, polydispersity of both LNPs remained <0.2. Both LNPs exhibited moderate to high encapsulation efficiency (<20% Dye accessible RNA) and >90% RNA recovery (FIG. 46C). Measured LNP size and PDI of inserted Lipid 15 LNPs are summarized in Table 34. As seen in FIG. 46D, preparation of anti-CD4 and/or anti-CD8 inserted mCherry and CAR LNPs with Lipid 15 resulted in LNP sizes <140 nm. Similarly, polydispersity of all inserted LNPs remained ≤0.2.

TABLE 33
Lipid 15 LNP size and PDI, zeta potential, and mRNA recovery pre-insertion.

| | | | Charge | Charge | mRNA | Dye |
|---|---|---|---|---|---|---|
| Ionizable Lipid, | Z-Avg. | | (ZP, mV); | (ZP, mV); | Recovery | accessible |
| LNP no. | Size (nm); | PDI (DLS); | pH 5.5 | pH 7.4 | (%); | (%); |
| Lipid 15, mCherry; | 95.15 | 0.115 | 21.8 | 0.619 | 106 | 12 |
| Lipid 15, CAR; | 112.35 | 0.129 | 21.9 | 0.935 | 113 | 17 |

TABLE 34
Lipid 15 LNP size and PDI post-insertion.

| Ionizable Lipid, LNP no., | Z-Avg. | PDI |
|---|---|---|
| mRNA, Targeting moiety | Size (nm); | (DLS); |
| Lipid 15, mCherry, TRX2 | 128.6 | 0.24 |
| Lipid 15, mCherry, Ibalizumab | 138.3 | 0.20 |
| Lipid 15, mCherry, TRX2/Ibalizumab (Density 1) | 134.2 | 0.17 |
| Lipid 15, mCherry, TRX2/Ibalizumab (Density 2) | 130.1 | 0.18 |
| Lipid 15, CAR, TRX2 | 125.2 | 0.15 |
| Lipid 15, CAR, Ibalizumab | 132.4 | 0.21 |
| Lipid 15, CAR, TRX2/Ibalizumab (Density 1) | 127.4 | 0.16 |
| Lipid 15, CAR, TRX2/Ibalizumab (Density 2) | 130.1 | 0.18 |

### Example 56. Viability, Mcherry, and Car Expression in Human CD3⁺, CD4⁺, and CD8⁺ Primary T-Cells Transfected with aCD4 (Ibalizumab) and aCD8 (TRX2) Targeted Lipid 15 LNPs

This example compares the CAR and mCherry protein expression resulting from Lipid 15 LNP's targeted with either CD4 (Ibalizumab) or CD8 (TRX2) Fab conjugates to those targeted with both CD4 (Ibalizumab) and CD8 (TRX2). Ibalizumab and TRX2 were inserted at 30 Fabs/LNP and 5 Fabs/LNP, respectively for single targeted LNPs. Dual targeted LNPs were inserted with 15 Fabs per LNP of Ibalizumab and 5 Fabs per LNP of TRX2 (Density 1) or 15 Fabs per LNP of Ibalizumab and 15 Fabs per LNP of TRX2 (Density 2). Particles produced were tested in vitro in primary human CD3+, CD4+, and CD8+ T cells to assess T-cell viability and mCherry and CAR expression, respectively. Both single and dual targeted LNPs were well tolerated by the CD3, CD4, and CD8 T-cell subsets in the in vitro experiment with T-cell viability values for treated samples being comparable to the untreated controls as seen in FIG. 47A. Single and dual targeted LNPs performed similarly both in terms of fraction of mCherry+ or CAR+ T-cells and the copies of mCherry or CAR protein produced on a per cell basis (reflected by MFI values) in the respective targeted subsets of T-cells (CD3+, CD4+, and CD8+) (FIG. 47B, FIG. 47C, FIG. 47D, and FIG. 47E).

### Example 57. Preparation of Fab-Lipid Conjugates to Enable In Vivo Targeting

This Example describes a method for the production of targeting group conjugates for incorporating LNPs (e.g., LNPs comprising an ionizable cationic lipid, where the ionizable cationic lipid is KC3 or Lipid 15) into targeted cells.

Fabs that bind to specific targets of a preferred cell type were conjugated to DSPE-PEG-maleimide via covalent coupling between the maleimide group and a C-terminal cysteine in the heavy chain (HC), following initial reduction of Fab. The protein was reconstituted with molecular biology grade water at 10 mg/mL in phosphate buffered saline (10 mM phosphate, 140 mM NaCl pH 7.4) and further diluted to 5 mg/mL in reduction buffer containing final concentration of 50 mM phosphate, 10 mM citrate, 75 mM NaCl, 5 mM EDTA pH 6.0 with 20 mM L-cysteine reducing agent and incubated for 1 hr at 25° C. with agitation under an Argon atmosphere. The reduced protein was immediately buffer exchanged to 99.9% into conjugation buffer 5 mM citrate, 140 mM NaCl, 1 mM EDTA pH 6.0 with a 10 kDa molecular-weight cutoff regenerated cellulose membrane in 24-well polypropylene filtration plate at room temperature using automated ultrafiltration/diafiltration buffer exchange (Unchained Labs, California, U.S.) equipped with HEPA air filtration system. The free sulfhydryl content after reduction and buffer exchange was measured to be <1.1 per Fab molecule after reduction and buffer exchange using Ellman's Reagent (5,5′-dithio-bis-[2-nitrobenzoic acid]) according to manufacturer's protocol (Thermo Fisher Scientific Peirce Biotechnology, Illinois, U.S.).

As soon as possible within 1 hr after buffer exchange the conjugation reaction was initiated by addition of a micellar suspension with 12 mg/mL DSPE-PEG-OCH₃ (NOF America, New York, U.S.) and 8 mg/mL DSPE-PEG-maleimide (NOF America, New York, U.S.) in molecular biology grade water. The conjugation reaction was carried out with a final concentration of 3.8 mg/mL Fab and an 8.25 molar excess of maleimide at for 4 hr at 25° C. with agitation under Argon atmosphere. The production of the resulting conjugate was monitored by HPLC and SDS-PAGE. The reaction was quenched in 1.0 mM L-cysteine at room temperature for 10 min and stored at 4° C. for 12-16 hr. The resulting crude conjugate reaction containing DSPE-PEG-Fab was simultaneously purified from free Fab and buffer exchanged to 99.9% into 10 mM citrate, 10% (w/v) sucrose pH 7.0 with a 100 kDa molecular-weight cutoff regenerated cellulose membrane in 24-well polypropylene filtration plate at room temperature using automated ultrafiltration/diafiltration buffer exchange (Unchained Labs, California, U.S.) equipped with HEPA air filtration system. Purity of the final conjugate from was assessed by HPLC and by SDS-PAGE. After quenching, the final micelle composition consists of a mixture of DSPE-PEG-Fab, DSPE-PEG-maleimide (cysteine terminated), and DSPE-PEG-OCH₃.

### INCORPORATION BY REFERENCE

Unless defined otherwise, all technical and scientific terms herein have the same meaning as commonly understood by one of ordinary skill in the art to which this invention belongs. Although any methods and materials, similar or equivalent to those described herein, can be used in the practice or testing of the present invention, the preferred methods and materials are described herein. All publications, scientific articles, patents, and patent publications cited are incorporated by reference herein in their entirety for all purposes.

The publications discussed herein are provided solely for their disclosure prior to the filing date of the present application. Nothing herein is to be construed as an admission that the present invention is not entitled to antedate such publication by virtue of prior invention.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein. While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

1: A compound of Formula (I): 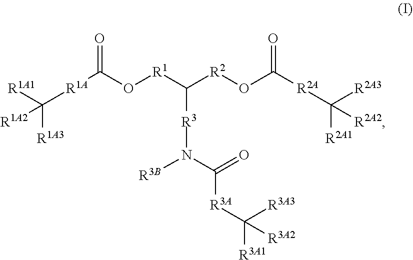
or a salt thereof, wherein:
R¹, R², and R³ are each independently a bond or C₁₋₃ alkylene;
R^{1A}, R^{2A}, and R^{3A} are each independently a bond or C₁₋₁₀ alkylene;
R^{1A1}, R^{1A2}, R^{1A3}, R^{2A1}, R^{2A2}, R^{2A3}, R^{3A1}, R^{3A2}, and R^{3A3} are each independently H, C₁₋₂₀ alkyl, C₁₋₂₀ alkenyl, —(CH₂)₀₋₁₀C(O)OR^{a1}, or —(CH₂)₀₋₁₀OC(O)R^{a2};
R^{a1} and R^{a2} are each independently C₁₋₂₀ alkyl or C₁₋₂₀ alkenyl;
R^{3B} is 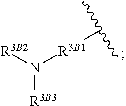
R^{3B1} is C₁₋₆ alkylene; and
R^{3B2} and R^{3B3} are each independently H or C₁₋₆ alkyl.

2: The compound of claim 1, or a salt thereof, wherein the compound is a compound of Formula (Ia):

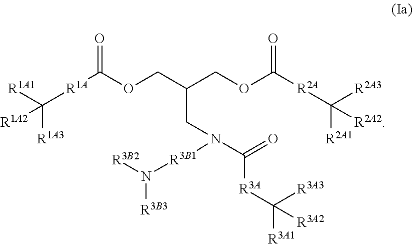

3: The compound of claim 1, or a salt thereof, wherein R^{3B1} is ethylene or propylene.

4: The compound of claim 1, or a salt thereof, wherein R^{3B2} and R^{3B3} are each independently H or C₁₋₆ alkyl optionally substituted with one or more substituents each independently selected from the group consisting of —OH and —O—(C₁₋₆ alkyl).

5: The compound of claim 4, or a salt thereof, wherein R^{3B2} and R^{3B3} are each independently methyl or ethyl, each optionally substituted with one or more —OH.

6: The compound of claim 5, or a salt thereof, wherein R^{3B2} and R^{3B3} are each unsubstituted methyl.

7: The compound of claim 1, or a salt thereof, wherein

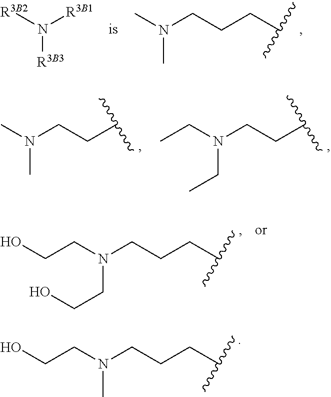

8: The compound of claim 1, or a salt thereof, wherein R¹, R², and R³ are each independently a bond or methylene.

9: The compound of claim 8, or a salt thereof, wherein R¹ and R² are each methylene and R³ is a bond.

10: The compound of claim 8, or a salt thereof, wherein R¹, R², and R³ are each methylene.

11: The compound of claim 1, or a salt thereof, wherein the compound is a compound of Formula (Ib):

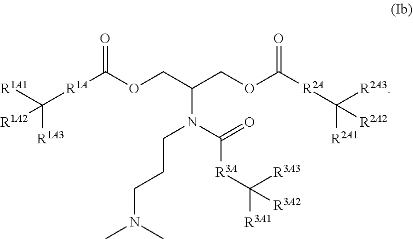

12: The compound of claim 1, or a salt thereof, wherein R^{1A}, R^{2A}, and R^{3A} are each independently a bond or —(CH₂)₁₋₁₀—.

13: The compound of claim 12, or a salt thereof, wherein R^{1A} and R^{2A} are each independently a bond, —CH₂—, —(CH₂)₂—, —(CH₂)₃—, —(CH₂)₄—, —(CH₂)₅—, —(CH₂)₆—, —(CH₂)₇—, or —(CH₂)₈—.

14: The compound of claim 13, or a salt thereof, wherein R^{1A} and R^{2A} are each independently a bond, —(CH₂)₂—, —(CH₂)₄—, —(CH₂)₆—, —(CH₂)₇—, or —(CH₂)₈—.

15: The compound of claim 12, or a salt thereof, wherein R^{3A} is a bond, —CH₂—, —(CH₂)₂—, or —(CH₂)₇—.

16: The compound of claim 1, or a salt thereof, wherein R^{1A1}, R^{1A2}, R^{1A3}, R^{2A1}, R^{2A2}, and R^{2A3} are each independently H, C₁₋₁₅ alkyl, —CH═CH—(C₁₋₁₅ alkyl), —CH═CH—CH₂—CH═CH—(C₁₋₁₀ alkyl), —(CH₂)₀₋₄C(O)OCH(C₁₋₁₀ alkyl)(C₁₋₁₅ alkyl), —(CH₂)₀₋₄OC(O)CH(C₁₋₁₀ alkyl)(C₁₋₁₅ alkyl), —(CH₂)₀₋₄C(O)OCH₂(C₁₋₁₅ alkyl), or —(CH₂)₀₋₄OC(O)CH₂(C₁₋₁₅ alkyl).

17: The compound of claim 16, or a salt thereof, wherein R^{1A1} and R^{2A1} are each independently —CH═CH—(C₁₋₁₅ alkyl), —CH═CH—CH₂—CH═CH—(C₁₋₁₀ alkyl), —(CH₂)₀₋₄C(O)OCH(C₁₋₁₀ alkyl)(C₁₋₁₅ alkyl), or —(CH₂)₀₋₄OC(O)CH(C₁₋₁₀ alkyl)(C₁₋₁₅ alkyl); and R^{1A2}, R^{1A3}, R^{2A2}, and R^{2A3} are each H.

18: The compound of claim 17, or a salt thereof, wherein R^{1A1} and R^{2A1} are each

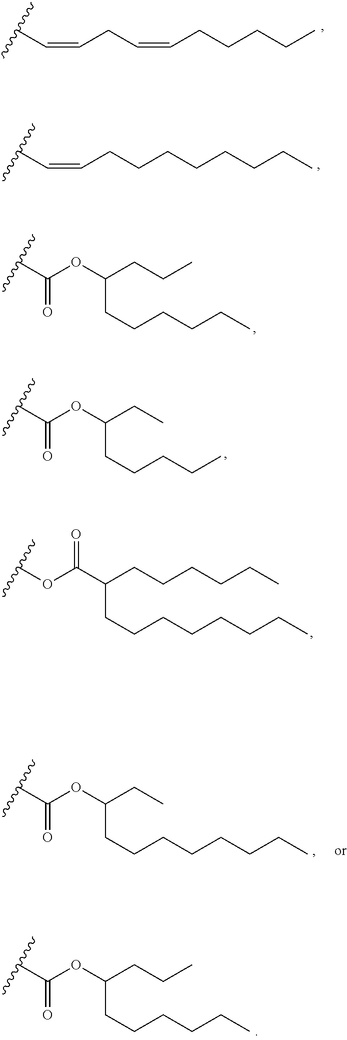

19: The compound of claim 16, or a salt thereof, wherein R^{1A1} and R^{2A1} are each C₁₋₁₅ alkyl; R^{1A2} and R^{2A2} are each C₁₋₁₅ alkyl; and R^{1A3} and R^{2A3} are each H.

20: The compound of claim 19, or a salt thereof, wherein R^{1A1} and R^{2A1} are each 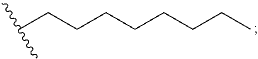 [SMILES: *CCCCCCCC]
and R^{1A2} and R^{2A2} are each

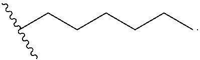 [SMILES: *CCCCCC]

21: The compound of claim 16, or a salt thereof, wherein R^{1A1} and R^{2A1} are each —(CH₂)₀₋₄OC(O)CH₂(C₁₋₁₅ alkyl); R^{2A1} and R^{2A2} are each —(CH₂)₀₋₄C(O)OCH₂(C₁₋₁₅ alkyl); and R^{1A3} and R^{2A3} are each H.

22: The compound of claim 21, or a salt thereof, wherein R^{1A1} and R^{2A1} are each 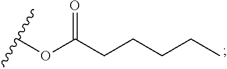 [SMILES: *OC(=O)CCCCC]
and R^{2A1} and R^{2A2} are each

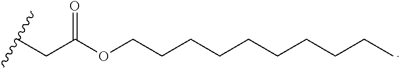 [SMILES: *CC(=O)OCCCCCCCCCC]

23: The compound of claim 16, or a salt thereof, wherein R^{1A1} and R^{2A1} are each —C(O)OCH₂(C₁₋₁₅ alkyl); R^{1A2} and R^{2A2} are each —(CH₂)₀₋₄C(O)OCH₂(C₁₋₁₅ alkyl); and R^{1A3} and R^{2A3} are each H.

24: The compound of claim 23, or a salt thereof, wherein R^{1A1} and R^{2A1} are each 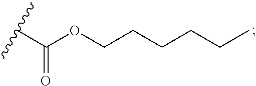 [SMILES: *C(=O)OCCCCCC]
and R^{1A2} and R^{2A2} are each 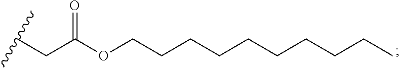 [SMILES: *CC(=O)OCCCCCCCCCC]
or wherein R^{1A1} and R^{2A1} are each 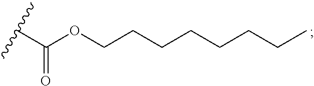 [SMILES: *C(=O)OCCCCCCCC]
and R^{2A1} and R^{2A2} are each

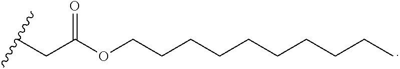 [SMILES: *CC(=O)OCCCCCCCCCC]

25: The compound of claim 1, or a salt thereof, wherein R^{3A1}, R^{3A2}, and R^{3A3} are each independently H, C₁₋₁₅ alkyl, —(CH₂)₀₋₄C(O)OCH(C₁₋₅ alkyl)(C₁₋₁₀ alkyl), —(CH₂)₀₋₄OC(O)CH(C₁₋₅ alkyl)(C₁₋₁₀ alkyl), —(CH₂)₀₋₄C(O)OCH₂(C₁₋₁₀ alkyl), or —(CH₂)₀₋₄OC(O)CH₂(C₁₋₁₀ alkyl).

26: The compound of claim 25, or a salt thereof, wherein R^{3A1} and R^{3A2} are each independently C₁₋₁₅ alkyl; and R^{3A3} is H.

27: The compound of claim 26, or a salt thereof, wherein R^{3A1} and R^{3A2} are each independently ethyl,

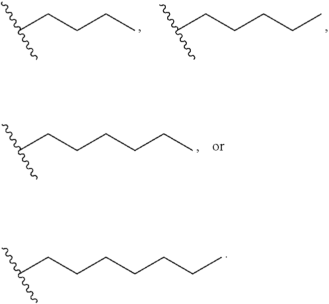

28: The compound of claim 25, or a salt thereof, wherein R^{3A1} is C₁₋₁₅ alkyl; and R^{3A2} and R^{3A3} are each H.

29: The compound of claim 28, or a salt thereof, wherein R^{3A1} is

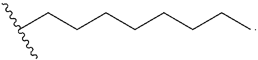 [SMILES: *CCCCCCCC]

30: The compound of claim 25, or a salt thereof, wherein R^{3A1} is —C(O)OCH(C₁₋₅ alkyl)(C₁₋₁₀ alkyl); and R^{3A2} and R^{3A3} are each H.

31: The compound of claim 30, or a salt thereof, wherein R^{3A1} is

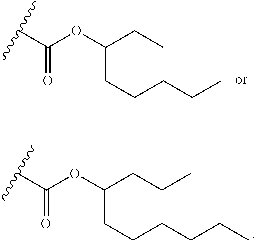 [SMILES: *C(=O)OC(CC)CCCCC ; *C(=O)OC(CCC)CCCCCC]

32: The compound of claim 25, or a salt thereof, wherein R^{3A1} is —(CH₂)₀₋₄OC(O)CH₂(C₁₋₁₀ alkyl); R^{3A2} is —(CH₂)₀₋₄(O)OCH₂(C₁₋₁₀ alkyl); and R^{3A3} is H.

33: The compound of claim 32, or a salt thereof, wherein R^{3A1} is 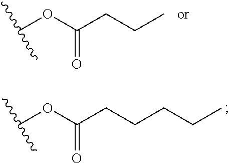
and R^{3A2} is

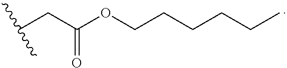 [SMILES: *CC(=O)OCCCCCC]

34: The compound of claim 25, or a salt thereof, wherein R^{3A1} is —(CH₂)₀₋₄C(O)OCH₂(C₁₋₁₀ alkyl); R^{3A2} is —(CH₂)₀₋₄C(O)OCH₂(C₁₋₁₀ alkyl); and R^{3A3} is H.

35: The compound of claim 34, or a salt thereof, wherein R^{3A1} is 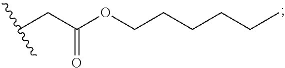 [SMILES: *CC(=O)OCCCCCC]
and R^{3A2} is or

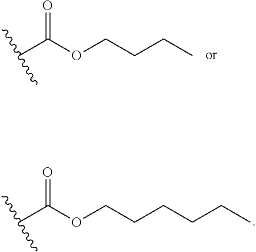

36: The compound of claim 25, or a salt thereof, wherein R^{3A1}, R^{3A2}, and R^{3A3} are each H.

37: The compound of claim 1, or a salt thereof, wherein R^{a1} and R^{a2} are each independently —(CH₂)₀₋₁₅CH₃ or —CH(C₁₋₁₀ alkyl)(C₁₋₁₅ alkyl).

38: The compound of claim 37, or a salt thereof, wherein R^{a1} and R² are each independently

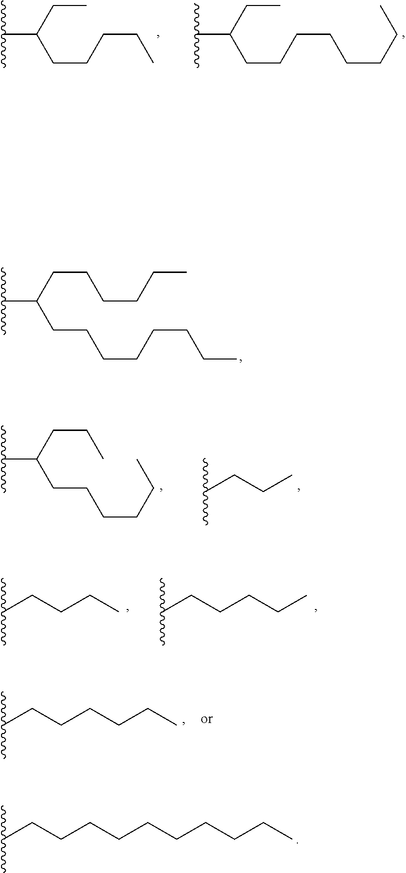

39: The compound of claim 1, or a salt thereof, wherein the compound is selected from Table 1.

40: The compound of claim 1, or a salt thereof, wherein the compound is

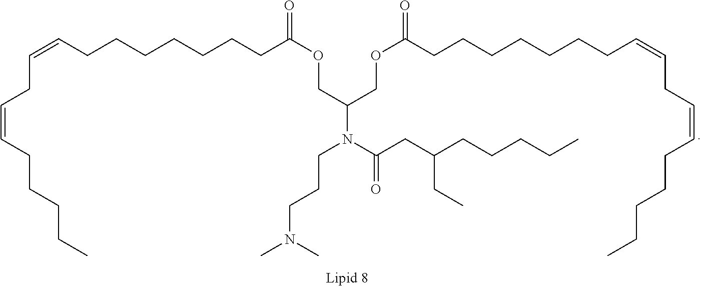 [SMILES: CCCCC/C=C\C/C=C\CCCCCCCC(=O)OCC(COC(=O)CCCCCCC/C=C\C/C=C\CCCCC)N(CCCN(C)C)C(=O)CC(CC)CCCCC]

41: The compound of claim 1, or a salt thereof, wherein the compound is

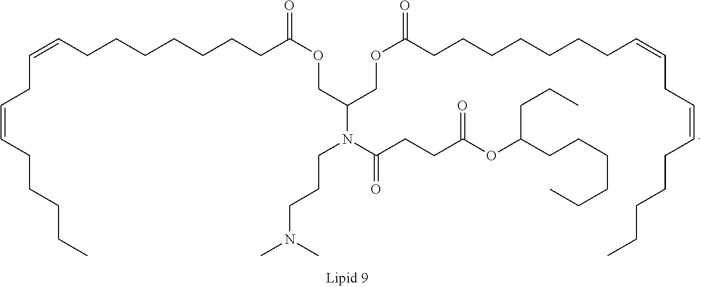 [SMILES: CCCCC/C=C\C/C=C\CCCCCCCC(=O)OCC(COC(=O)CCCCCCC/C=C\C/C=C\CCCCC)N(CCCN(C)C)C(=O)CCC(=O)OC(CCC)CCCCCC]

42: The compound of claim 1, or a salt thereof, wherein the compound is

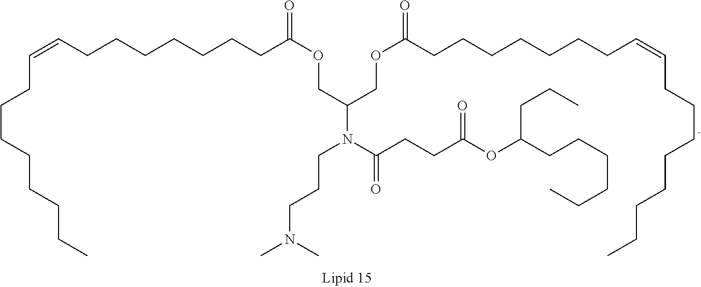 [SMILES: CCCCCCCC/C=C\CCCCCCCC(=O)OCC(COC(=O)CCCCCCC/C=C\CCCCCCCC)N(CCCN(C)C)C(=O)CCC(=O)OC(CCC)CCCCCC]

43: A lipid nanoparticle (LNP) comprising a lipid blend for targeted delivery of a nucleic acid into an immune cell, the lipid blend comprising:
(a) a lipid-immune cell targeting group conjugate comprising the compound of Formula (II): [Lipid]-[optional linker]-[immune cell targeting group], and
(b) an ionizable cationic lipid comprising the compound of claim 1, or a salt thereof,
wherein the LNP further comprises a nucleic acid disposed therein.

44-137. (canceled)

138: A method of targeting the delivery of a nucleic acid to an immune cell of a subject, comprising contacting the immune cell with the LNP of claim 43, wherein the LNP comprises the nucleic acid.

139: A method of expressing a polypeptide of interest in a targeted immune cell of a subject, comprising contacting the immune cell with the LNP of claim 43, wherein the LNP comprises a nucleic acid encoding the polypeptide.

140: A method of modulating cellular function of a target immune cell of a subject, comprising administering to the subject the LNP of claim 43, wherein the LNP comprises a nucleic acid modulates the cellular function of the immune cell.

141: A method of treating, ameliorating, or preventing a symptom of a disorder or disease in a subject in need thereof, comprising administering to the subject the LNP of claim 43 for delivering a nucleic acid into an immune cell of the subject, wherein the LNP comprises the nucleic acid.

142-194. (canceled)
